SEQUENCE LISTING (224 sequences)

SEQ ID NO: 1 - protein, 389 aa, Bacillus cereus
MKSPNFQLSLQTSNLIIGFMVWVILSSLMPYIKVDIPLTAGQISMVTAVPVILGSVLRIPIGYWTNRFGARKLFFISFILLLLPVFYISVANSMMDLIIGGLFVGIGGAVFSVGVTSLPKYFPKESHGFVNGIYGVGNAGTAITSFLAPVIATSVGWRTTVQCYLVLLAAFALMNFLLGDRKEKKVNTPLMEQIKGVYKNEKLWFLCIFYFLTFGSFVAFTVYLPNFLVSHFGLEKVDAGMRTAGFIVLATIMRPIGGWLGDKFNPFKILIFVFIGLTLSGIILSFMPSMNVYTFGCLLVAFCAGIGNGTIFKLVPMYFSEQAGIVNGLVSALGGLGGFFPPLILTLLFQLTGHYAIGFMALSEVALACLIITVWMYSQEKLLVMLKNH

SEQ ID NO: 2 - protein, 801 aa, Bacillus cereus
MKKRLVMIGNGMAGIRCMEEILKHDSDSYEITIFGDEPHPNYNRIMLSHVLQGKTNIQDIIMNEYSWYEENEITLYTNERVQSINREEKIIITEKKRTLTYDKLIIATGSSAFILPVEGSALSGVTGFRTIEDTQFMIDTAKEKKKAVVIGGGLLGLEAARGLIDLGMDVHVVHLMPSLMEQQLDTKAAALLREDLEAQGMKFLMEKKTVKILGTDHVEGIQFEDGEVVDCDLIVMAVGIRPNTQIAKDAGLIVNRGIVVNDYMLTNDESIYAVGECAEHDGIAYGLVAPLYEQGAILAKHITNLQTDGYSGSIVGTQLKVAGCDLFSAGQIYEDDQTKAISIFNECKRSYKKILIRDNKVVGIVLYGDTADGTRLFSILKKEEDIQEYTPASILHKAGEECELDVATMSADDTICGCNGVTKGTIVHAILEQELKTFEEVKACTKAAGSCGKCRPLVEQVLSHTLGDAFDASAQSTGMCGCTPLSRDEVVAAIHEKGLKSPKEVRNVLGFVHEDGCSKCRPALNYYLRMAIPEEYEDDKSSRFVNERMNGNIQHDGTFSVIPRMYGGVTTADDLMKIAEVAKKYDVPLVKITGASRIGLYGVKKQDLPNVWAELNMASGYAYSKSLRNVKSCVGSRFCRFGTKDSLGLGMLLEQSLEMVDTPHKMKMGVTGCPRNCAEVLTKDFGVVCVENGYQLYIGGNGGTEVREADFVIIVPTEDDVLRIATAYMQYYRETGIYGERTAYWTERLGFDHIKEILQDANMVTKLNERFQKARGTYKEAWGQALETKSLKAMYEVETVK

SEQ ID NO: 3 - protein, 105 aa, Bacillus cereus
MIQTKEKIKVMRAEDLPIQIGKEVQMKGMSFALFRLSNGDIRAVENRCPHKKGPLAEGIVSGEFVFCPLHDWKISLLTGEVQKPDDGCIQTYEVEVIDGDIYIYM

SEQ ID NO: 4 - protein, 323 aa, Bacillus cereus
MCGCTPLSRDEVIAAIHEKGLKSPKEVRNVLGFAHEDGCSKCRPALNYYLRMTIPEEYEDDKSSRFVNERMNGNIQHDGTFSVIPRMYGGVTTADDLMKIAEFAKKYDVPLVKITGASRIGLYGVKKQDLPNVWAELNMTSGYAYSKSLRNVKSCVGSRFCRFGTKDSLGLGMLLEQSLEMVDTPHKIKMGVTGCPRNCAEVLTKDFGIVCVENGYQLYIGGNGGTEVREADFVMIVPTEDDVLRIATAYMQYYRETGIYGERTAYWTERLGFDHIKEILQDANMVTKLNERFQTARGTYKEAWGQALETKSLKAMYGVETVK

SEQ ID NO: 5 - protein, 105 aa, Bacillus cereus
MLQSKEKFKIMRAEDLPFQIGKEVQMKGISIILFRLLNGDIRAVENHCPHKNGPLAEGIVSGEFVFFPLHDWKISLVTGEVQKPDDGCIQTYEVEVIDGDIYIYM

SEQ ID NO: 6 - protein, 1227 aa, Bacillus cereus
MKKKTSALMRRLKYFSPIDRYNDNHTQETYEDREWENVYRKRWQHDKVIRSTHGVNCTGSCSWNIYVKDGIVTWEGQELNYPTTGPDMPDFEPRGCPRGASFSWYIYSPLRVKYPYVRGVLWNMWQEELQNNESPLEAWKSIVENREKARTYKQARGKGGFIRVNWDEVLQLVSASLLYTVMKYGPDRNVGFSPIPAMSMLSHAAGSRFMQLMGGPMLSFYDWYADLPPASPQIWGDQTDVPESSDWYNSGYIMTWGSNVPMTRTPDAHFLAEVRYKGTKVVSVSPDFAESTKFADDWISVKQGTDGALAMAMGHVILQEFYVDNQVEYFTKYVKQYTDFPFFVTLKQKGDQFVADRFLNASDIGRETKLGEWKPVLWNENTNDFATPHGTMGSRWDNEKKWNLRLEDEETGEKIDPRLSLLGMEDSVQTVQIPYFSDDGNKILERTIPVKKVMTEEGELFVTTVYDLTLANYGVNRGVGGQEPKDFNDDIPFTPAWQEKMTGVKRELIIQIAREFAQNAVDTNGRSMIIVGAGINHWFNSDTIYRAVLNLVLLVGAQGVNGGGWAHYVGQEKLRPAEGWQTIAMAKDWQGPPKLQNGTSFFYFVTDQWRYEDTPVGHLASPVEGNSRYQHHGDYNVLAARLGWLPSYPTFEKNGIELYKEAVAAGATTQEEIGKYVAQKLKEKELKFAIEDPDNKNNFPRNLFVWRANLISSSGKGHEYFLKHLLGTTNGLMNDDSDSLRPEEIKWHEEAPEGKLDLLINLDFRMAGTALYSDIVLPASTWYEKHDLSSTDMHPFVHPFNPAIGSPWEARSDWDIFTSLSKAVSDLAKKIDLEPMKEVVATPLLHDTPQELAQPLGKIKDWSKGECEPIPGKTMPQIHVVERDYKTIYDKMTALGPNAGKQPIGTKGISWSAEKEYEQLKSKLGVVRTDSIAKGCPDIKEAINAAEAVLTLSSTTNGHMAVKAWEALEKQTDLKLRDLAEEREEECFTFEQITAQPKTVITSPAFTGSEKGGRRYSPFTTNVERLIPWRTITGRQSFYLDHDMMKEFGETMATFKPILQHKPFRKSRPEVEGKEITLNYLTPHNKWSIHSMYFDSLPMLTLFRGGPTVWMNKEDAAEAGVADNDWIECFNRNGVVVARAVVTHRIPRGMAFMHHAQDRHINVPGTKLTSNRGGTHNSPTRIHVKPTHMIGGYGQLSYGFNYYGPTGNQRDLNVVIRKLKEVDWLED

SEQ ID NO: 7 - protein, 491 aa, Bacillus cereus
MKIKAQVGMVMNLDKCIGCHTCSVTCKNTWTNRPGAEYMYFNNVETKPGIGYPKQWEDQEKYKGGWELKNGEIQLKSGSKMKRLMNIFHNPDQPTIDDYFEPWNYDYETLTNSPQRKHQPVARPKSAITGEFIDKIEWGPNWEDDLAGGHITGLQDPNVKKMEEEIKTDFENVFMMYLPRICEHCMNPSCVSSCPSGAMYKREEDGIVLVDQNACRAWRFCVSSCPYKKVYFNWQTNKAEKCTMCFPRIEAGMPTICSETCVGRIRYIGVMLYDADKVKEAASVEDEKDLYESQLTVFLDPNDPEIAAEAKKQGIPEEWIKAAQQSPIYKMIIDWKIALPLHPEYRTMPMVWYIPPLSPIMNMVEGKGSNWQAEEVFPAIDNMRIPIQYLANLLTAGDESHIRLTLKKMAVMRTYMRALQINKEPNEAVLKELGLAKQDVEDMYRLLAIAKYKDRFVIPTSHREQVADLYSEQGSCGLSFTGGPGSCMTIS

SEQ ID NO: 8 - protein, 176 aa, Bacillus cereus
MRQSLQTAFSCSSFLLSYPELGWREAVTELQEEVETIKQEDVKASLTAFIKQVLNKTNDQLIDSYVYTFDFGKKTNMYLTYMNTGEQRERGIELLELKQHYKKSGFEVTDKELPDYLPLLLEFFANANEIDSEPIMSKYTENIQALHVQLKEADSMYEPILAAVLLAIETWGVQTN

SEQ ID NO: 9 - protein, 229 aa, Bacillus cereus
MMDQFLWVLFPYIIFAIFIGGHIFRYNYDQFGWTSKSSELLEKKMLRVGSLLFHFGIMFVIGGHVMGILIPEAVYRSIGISEHMYHVVAISFGLPAGVASIIGLIILTYRRVTVKRIIATSTKGDYIALILLLIVMLAGLSSTFLNIDSKGFDYRTTIGPWFRSLFIFQPKVEYMMEVPIWFKIHILAGMGLFAVWPFTRLVHVFSAPIKYVSRSYVIYRRRIPNELKK

SEQ ID NO: 10 - protein, 356 aa, Bacillus cereus
MSKTKQLIEEASNFITICYKELHKEQLIEERIKEIQIEIEKTGTYEHTFEELVHGSRMAWRNSNRCIGRLFWSKMHILDAREVNDEEGVYNALIHHIKYATNDGKVKPTITIFKQYQGEENNIRIYNHQLIRYAGYKTETGVIGDSHSATFTDFCQELGWQGEGTNYDVLPLVFSIDGKAPIYKEIPREEVKEVPIEHPEYPISSLGVKWYGVPMISDMRLEIGGISYTAAPFNGWYMGTEIGARNLADHDRYNLLPAVAEMMDLDTSRNGTLWKDKALIELNIAVLHSFKKQGVSIVDHHTAAQQFQQFEKQEAACGRVVTGNWVWLIPPLSPATTHIYHKPYPNEILKPNFFHK

SEQ ID NO: 11 - protein, 356 aa, Bacillus cereus; identical to SEQ ID NO: 10

SEQ ID NO: 12 - protein, 399 aa, Bacillus cereus
MNEHIAVKELSEKLLEDYKTESSFFFASPTRTILAEGEFTTVKHREIESFPELVQAKLSNAKQAGNPNPIVVGALPFDRRKEVQLIVPEYSRISERLQLDTTNQLETNENVTFEMTPVPDPEVYMNGVKQGIEKIQDGDLKKIVLSRSLDVKSSEKIDKQKLLRELAEHNKHGYTFAVNLPKDEKENSKTLIGASPELLVSRNGMQVISNPLAGSRPRSEDPVEDKRRAEELLSSPKDLHEHAVVVEAVAAALRPYCHTLHVPEKPSVIHSEAMWHLSTEVKGELKDPNTSSLQLAIALHPTPAVCGTPMEKAREAIQHIEPFDREFFTGMLGWSDLNGDGEWIVTIRCAEVQENTLRLYAGAGVVAESKPEDELAETSAKFQTMLKALGLRDSSLNEK

SEQ ID NO: 13 - protein, 464 aa, Bacillus cereus
MIQTKQKGLQEVLSAAIKHATDEKILVSFVKQIDWMDPLLFYAAGKRIALENRCYFADPAQHVIFAGIGSVFTIANSSHKRFQAARDEWDKVKEKAFVQREKYEFGTGPLLFGGFSFDQEKEKTDLWKEFDDTTFSLPAFLLTVKNEKAWLTMNTFVSATDCAETLYNEIVSLEEKIFGESKCALEGSKLTVTSKVEVDPKGWMKAIEKVQDEMKQGNVQKVVLARELKVEMDHHIDSALVLEALRIGQPDCYVFSFDYKGACFLGATPERLIRKEDEKFTSMCLAGSTGHGQSIEESKRNSNALLHDEKNLAEHGYVVNMIRSVLNEHCEYVNIPESPGLLTTKNLIHLYTPVEAKGTASLLTMVEELHPTPALGGTPRLEAMKLIRDVELLDRGLYGAPIGWIDDEGNGEFAVALRCGLLNGEKASLFAGCGIVIDSVPQLEYEETSLKFRPMLGALEELMK

SEQ ID NO: 14 - protein, 358 aa, Bacillus cereus
MANHELDQLRKQVDEINLQLLHLLNKRGEIVQKIGEQKQVQGTKRFDPVREREVLDMIAEHNEGPFETSTVQHIFKTIFKASLELQEDDNRKALLVSRKKKQENTIVDVKGELIGNGTQTFIMGPCAVESLEQVRQVGQAMKDQGLKLMRGGAFKPRTSPYDFQGLGVEGLQILRQVADEFDLAIISEILNPNDVEMALDYVDVIQVGARNMQNFDLLRAVGKVNKPVLLKRGLAATIDEFINAAEYIIAQGNDQIILCERGIRTYERATRNTLDISAVPILKKETHLPVIVDVTHSTGRRDLLLPTAKAALAIGADAVMAEVHPDPAVALSDSAQQMDIPEFHRFMDELKGFKNKLS

SEQ ID NO: 15 - protein, 357 aa, Bacillus cereus
MASQQLGRLRSEIDQLNLQILELLNERGRLVQEVGNLKEVQGVKRFDPVRERNMLDLIAENNNGPFETSTLQHIFKQIFQAGLELQEDDHRKALLVSRKKKTEDTIVEINGEKIGDGNQHFIMGPCAVESYEQVRQVAEAMKEQGLKLMRGGAFKPRTSPYDFQGLGLEGLQILRQVADEYDLAVISEILNPNDIEMSLDYVDVIQIGARNMQNFDLLRAAGAVNKPVLLKRGLSATIEEFINAAEYIMAKGNGNIILCERGIRTYERATRNTLDISAVPILKKETHLPVVVDVTHSTGRRDLLLPTAKAAMAIGADAIMAEVHPDPAVALSDVAQQMNIPQFNDFMNELKSFGSKL

SEQ ID NO: 16 - protein, 2385 aa, Bacillus cereus
MPNSQKIRHSLSSAQSGMWFAQQLDPLNPIYNTGEYVEINGNIHQEIFELAVRKVVIEAEALHVRFEEDEIGPWQVIEESQFHMHFIDVRKEENPEEAAKVWMKNDLSMPVDLKKDTLFTEALIQVENNRFFWYQRIHHIVMDGYGFSLLSQKVANEYTSLIEETNKNEKPFGSLTKVVQEDIEYRDSKKFQEDRTFWLEKFADEPEVVSLAERAPRTSNGFSRETAYLSSSSTKTLLEDINISLTSWPEFIVAVTSIYMHKLTGANDIVLGLPMMGRLGSVSIHTPSMVMNLVPLRITVTPNITLAELLQQVSKEIRDVRRHYKYRHEELRRDLKLLGENQRLFGPLVNVMPFDYGLNFAGNRGITHNLSAGPVDDLSINVYKRFDQNKLMIHFDANPEVYNGAELALHKERFMSLFELVVNNYEKNESIGKINITLPEENHKVLLEWNETKEDDELLSLPISFEKQVQKNPNKLAITCDGVNLTYKELNARANELAHYLVEEGIRPNQFVALVFPRSIEMVVSMLAVLKAGAAYLPIDPEYPAERINYIVNDAKPVCIITHSSVSSKLVIENDMKKIVLDEEETKLALHTYSRMNIACKNDVSLLNPAYTIYTSGSTGNPKGVIVPMRGLSNFLMAMQQKFSLNENDHLLAVTTFAFDISALEIYLPLISGASLTIAQKEDIQEPSALTTLLQEERVTIMQATPTLWQALVTDYPEKLQGLNILVGGEALPEHLANKLKELGCSITNLYGPTETTIWSTFMNIDEGEKGIPPIGKPICNTEVYVLDAGLQPVPPGVIGELYIAGEGLASGYLGKPELTAERFIANPYGESGKRMYRTGDLVKWRSDGALEYISRADHQIKIRGFRIELAEIETVLQRHENIQQAVVIVREDRPNDKRIIAYIVAEEKEPINLSEIRSYVSESLANYMIPSAFVVLEELPLTPNGKVDRKKLPAPDFNRMDNERVARNPKEEILCDLFAEVLGVSRISIDDNFFEMGGHSLLASRLMARIRETLSVELGIGKLFESPTVAELAKQLNHAKSARPAIQKASRPNEVPLSFAQRRLWFLNCLEGPSPTYNIPLVIRMNGILNREALQGAFYDVVEKHETLRTIFPNVLGSSYQRILDMENLNLEMVITNTCKDELESVFSEAVRYSFNLDFEPAVRLQLFTVSENEHVLLILLHHIVGDGWSLQPLTRDFTAAYKARCQGDRVQLETLSVQYADYALWQQQLLGDETTPESLISTQLDFWKEELKGLPDQMELPTDFQRPIETSYRGETIHFHIDEGMHSRLVELARKNGVSLFMVLQAGLSALFTRLGAGTDIPIGSPIAGRNDDVLSDIVGLFVNTLVLRTNTSGDPSFKELLNRVKQVNLAAYENQDVPFERLVEVLNPVRTRNSHPLFQVMLAFQNTPEAIFDAPDIEASLEIQSVGSAKFDLTFEISESNGVDGTPNGLHGLLEFSTDLYKRETVQKLIERFILLLDDAATNPDQSIGRLEILTVAEKNTVLEKWNGGFQIAPEMTLPQLFEKQVHINPNSIAVVFEDKKLTYEELNRKANKIARFLIAKGIGPDQLVALAMPRSLNMVVSLLAVLKAGAGYLPLDPDYPADRISFMLHDAKPTCVLTNSEVEIECNEALKVLVDDVKVIAEVEKYSEENIDEVERIKPLSPSHIAYVIYTSGSTGRPKGVMIPHQNVVRLLGATDHWFQFDGNDVWTMFHSYAFDFSVWEIWGPLLYGGRLVVVPHTVSRSPKEFLQLLVKEKVTVLNQTPSAFYQLMQADRENEEIGQKLSLRYVVFGGEALELSRLEDWYSRHPHNAPKLINMYGITETTVHVSYIELDETIVSLRANSLIGCSIPDLKVYVLDNYLQPVPPGVVGEMYVAGAGLARGYLGRAGLTAERFIADPFGKPGTRMYRTGDLARWRKDGTLDYIGRADHQIKIRGFRIELGEIEAVIMKHPKVEQVVVIVREDQPGDKRLVSYIVASNNEAIDTNEMRQFASGSLPDYMVPYDFVVVNELPLTPNGKLDRKALPAPEFIASSSSRGPRTPQEEMLCDLFTEVLSVPQIGIDDGFFDLGGHSLLAVQLMSRMKEALGVELNIGTLFAAPTVAGLAERLEMGNGQSALDVLLSLRASGDQLPLFCVHPAGGLSWCYAGLMKSLGTDYPIYGVQARGIAKNEELPKSLEEMAADYLKQVREVQPHGPYRLLGWSLGGNVVHAMAAQLQNEGEEVELLVMLDSYPGHFLPNTEAPTEEEALIALLALGGYDPDNMDGKPLTMESAVEILRKDGSALASLEEETILNLKETYVNSVGLLGKYVPKVYNGDILFFRSTVIPDWFDPISPNTWLNYLDGQIVQHDIDCRHKDLCQPGPLTEIGQVLAKYLQNKKGVSRV

SEQ ID NO: 17 - protein, 538 aa, Bacillus cereus
MLVGYTEWPKEFADRYREAGCWLGETFGGVLRERAEKYGDRIAVVSGKKHITYSELNKKVDRLAAGLLNLGIKKEDRVVIQLPNIIEFFEICFALFRIGALPVFALPSHRSSEISYFCEFGEASAYVISDKALGFDYRKLAREVKEKVPTLQHVIVVGEEEEFVNINDLYMDPVSLPEVQPSDVAFLQLSGGTTGLSKLIPRTHDDYIYSLRVSAEICNLNAESVYMAVLPVAHNYPMSSPGTFGTFYAGGKVVLATGGSPDEAFALIEKEKVTITALVPPLAMIWLDAASSRNADLSSLEVIQVGGAKFSAEVAKRIRPTFGCTLQQVFGMAEGLVNYTRLDDPEEFIIYTQGRPMSALDEVRVVDENDNDVQPGEVGSLLTRGPYTIRGYYKAEEHNARSFTKDGFYRTGDLVKVNEQGYIIVEGRDKDQINRGGEKVAAEEVENHLLAHDSVHDVAIVSMPDDYLGERTCAFVIARGQVPAVSELKMFLRERGIAAYKIPDRIEFIEAFPQTGVGKVSKKELRKVIAEKLITVKQ

SEQ ID NO: 18 - protein, 297 aa, Bacillus cereus
MAIPSISVYKMPIESELPKNKVNWTPDPKRAVLLIHDMQEYFLDAYSDKESPKVELISNIKMIREKCKELGIPVVYTAQPGGQTLEQRGLLQDFWGDGIPAGPDKKKIVDELTPDEDDIFLTKWRYSAFKKTNLLEILNEQGKDQLIICGIYAHIGCLLTACEAFMDGIEPFFVADAVADFSLEHHKQALEYASNRCAVTTSTNLLLNDLQSVKDDESEGITIQEVHELVAQLLREPVESIDIDEDLLNRGLDSVRIMSLVEKWRREGKEITFAHLAERPTVAGWYSLLSSQTAQVL

SEQ ID NO: 19 - protein, 258 aa, Bacillus cereus
MGVEKIKAAFENGKKAFIPYVMGGDGGFEKLKERIRFLDEAGASIVEIGIPFSDPVADGPTIQRAGKRALDSGVTVKGIFQALIEVRKEVQIPFVLMTYLNPVLAFGKERFIENCIEAGVDGIIVPDLPYEEQDIIAPLLREANVALIPLVTITSPIERIEKITSESKGFVYAVTVAGVTGVRQNFKEEIHSYLEKVKSHVNLPVVAGFGISTKEHVEEMVTICDGVVVGSKIIELLENEKQEEICELIYATKQKEEA

SEQ ID NO: 20 - protein, 397 aa, Bacillus cereus
MNYAYPDEKGHYGIYGGRYVPETLMQSVLELEEAYKEAMQDEAFQKELNHYLKTYVGRETPLYFAENLTKYCGGAKIYLKREDLNHTGAHKINNTIGQALLAVRMGKKKVVAETGAGQHGVATATVCALLGLECVIFMGEEDVRRQKLNVFRMELLGAKVESVAAGSGTLKDAVNEALRYWVSHVHDTHYIMGSVLGPHPFPQIVRDFQSVIGNETKKQYEELEGKLPEAVVACIGGGSNAMGMFYPFVHDEEVALYGVEAAGKGVHTEKHAATLTKGSVGVLHGSMMYLLQNEEGQIQEAHSISAGLDYPGVGPEHSLLKDIGRVSYQSITDEEALEAFQLLTKKEGIIPALESSHAVAYALKLAPKMKEDEGLVICLSGRGDKDVESIKRYMEEV

SEQ ID NO: 21 - protein, 253 aa, Bacillus cereus
MGTILDKIVEQKKKEVAELYEIYTPVKAKRKAHSLVEALQQFTVIAEVKRASPSKGDINLHVDVRKQVGTYEKCGAGAVSVLTDGQFFKGSFHDLQTAREESNIPLLCKDFIIDKIQIDRAYEAGADIILLIVAALTKEKLKELYSYVLEKGLEAIVEVHDEQELETAIVLNPHVIGINNRNLKTFEVDLSQTEKLGKRLNEEKLLWISESGIHSKEDIIRVKRAGAKGVLVGEALMTSSSISSFFEDCKVNI

SEQ ID NO: 22 - protein, 341 aa, Bacillus cereus
MNNYLRKLVEGQHLTEEEMYKAGLLLLSENILESEIAAFLVLLKAKGETAEEIYGLVRALREKALPFSNHIQGAMDNCGTGGDGAQTFNISTTSAFVLAGAGVKVAKHGNRAVSSKTGSADLLEELGVNISSTPNEIDYLLEHVGIAFLFAPAMHPALRRIMKIRKELNVPTIFNLIGPLTNPVNLETQFVGIYKRDMLLPVAQVLQKLGRKQALVVNGSGFLDEASLQGENHVVLLKDNEIVEMSIDPEKYGFSRVKNEEIRGGNSKENAKITLEVLSGEKSVYRDTVLLNAGLALFANGKTETIEEGIKLAAHSIDSGKALTKLNLLIAASNEKLERVN

SEQ ID NO: 23 - protein, 253 aa, Bacillus cereus; identical to SEQ ID NO: 21

SEQ ID NO: 24 - protein, 341 aa, Bacillus cereus; identical to SEQ ID NO: 22

SEQ ID NO: 25 - protein, 472 aa, Bacillus cereus
MMTKEEFIKQKRERKTFLVITEEEGDSITPISLYRRMKGKKKFLLESSQLHQDKGRYSYLGCNPYGEVKSVGTEVERTIFGRAEKLQGNVLQVLEEVIAPSQVDSPFPFCGGAVGYIGYDVIRQYENIGADLHDPLNIPEVHLLLYREFIVYDHLRQKLSFVYVCREDDSTDYEEVYERLRVYKEEVLQGEEAEVNAIQSPLSFTSSITEKEFCEMVEIAKEYIRAGDIFQVVLSQRLQSECIGDPFALYRKLRIANPSPYMFYIDFQDYVVLGSSPESLLSVREDKVMTNPIAGTRPRGKTKREDEEIAKELLGNEKERAEHMMLVDLGRNDIGRVSEIGSVTIDKYMKVEKYSHVMHIVSEVYGTLRKQMGGFDALAYCLPAGTVSGAPKIRAMEIINELENEKRNVYAGAVGYVSFSGNLDMALAIRTMVVKDEKAYVQAGAGVVYDSDPVAEYEETLNKARALLEVMK

SEQ ID NO: 26 - protein, 204 aa, Bacillus cereus
MKVKICGITDMETAKSACEYGADALGFVFAESKRKITPKRAKEIIQELPANVLKIGVFVNESVEVIQKIADECGLTHVQLHGDEDNYQIRRLNIPSIKSLGVTSESDMKNAQGYETDYILFDSPKEKFHGGNGKTFSWELLGHMPKELRKKTILAGGLNALNIEEAIRTVRPYMVDVSSGVETEGKKDVEKIKQFIIKAKECSK

SEQ ID NO: 27 - protein, 536 aa, Bacillus cereus
MKKWVKVTLSITGGIVLLACAGGYYVYKNYFPKESERIVYDKERVLQPIHNQLKGINIENVKIKEKEVVNATVDELQKMIDDGKLSYEELTSIYLFRIQEHDQNGITLNSVTEINPNAMEEARKLDQERGRNKNSNLYGIPVVVKDNVQTEKVMPTSAGTYVLKDWIADQDATIVKQLKEEGAFVLGKANMSEWANYLSFTMPSGYSGKKGQNLNPYGPITFDTSGSSSGSATVVAADFAPLAIGTETTGSIVAPAAQQSVVGLRPSLGMVSRTGIIPLVETLDTAGPMARTVKDAATLFNAMIGYDEKDVMTEKMKDKERIDYTKDLSIDGLKGKKIGLLFSVDQQDENRKAVAEKIRKDLQDAGAILTDNIQLSAEGVDNLQTLEYEFKHNVNDYLSQQKNVPVKSLEEIIAFNKKDSKRRIKYGQTLIEGSEKSVITKEEFENVVQTSQENARKELDRYLVEKGLDALVMINNDEVLLSAVAGYPELAVPAGYDKNGEPIGVVFVGKQFGEKELFNIGYAYEQQSKNRKLPSL

SEQ ID NO: 28 - protein, 485 aa, Bacillus cereus
MSLFDHSVSELHKKLNSKEISVTDLVEESYKRIADVEDNVKAFLTLDEENARAKAKELDAKIGAEDNGLLFGMPIGVKDNIVTNGLRTTCASKILANFDPIYDATVVQKLKAADTITIGKLNMDEFAMGSSNENSGFYATKNPWNLDYVPGGSSGGSAAAVAAGEVLFSLGSDTGGSIRQPAAYCGVVGLKPTYGRVSRYGLVAFASSLDQIGPITRTVEDNAYLLQAISGIDRMDATSANVEVGNYLAGLTGDVKGLRIAVPKEYLGEGVGEEARESVLAALKVLEGMGATWEEVSLPHSKYALATYYLLSSSEASANLSRFDGVRYGVRSDNVNNLLDLYKNTRSEGFGDEVKRRIMLGTFALSSGYYDAYYKKAQQVRTLIKNDFENVFANYDVIIGPTTPTPAFKVGEKVDDPMTMYANDILTIPVNLAGVPAISVPCGFGANNMPLGLQIIGKHFDEATIYRVAHAFEQATDYHTKKASL

SEQ ID NO: 29 - protein, 558 aa, Bacillus cereus
MKKQYTVSTYLLDRLHELGIEHIFGVPGDYNLAFLDDVVAHKNLKWIGNCNELNAAYAADGYARIKGIAALITTFGVGELSAINGIAGSYAENVPVIKITGTPPTKVMENGAIVHHTLGDGKFDHFSNMYREITIAQTNVTPEHAAEEIDRVLRACWNEKRPGHINLPIDVYNKPINKPTEPIINKPILSNKEALNKMLLHAISKINSAKKPVILADFEVDRFHAKESLHQFVEKTGFPIATFSMGKGIFPEKHPQFIGVYTGDVSSPYLRKRIDESDCIISIGVKLTDTITGGFTQGFTKEQVIEIHPYTVKIIDKKYGPVVMQDVLQHLSDSIEHRNKDTLDVKPFILESSPFTEEFNPKAQMVTQKRFWQQMYHFLQENDVLIVEQGTPFFGSAAIPLPNNTAYVGQPLWGSIGYTLPALLGTQLANLSRRNILIIGDGSFQVTAQELSTILRQNLKPIIFLINNNGYTVERAIHGQNQLYNDIQMWDYNKLSMVFGSEEKSLTFKVENEAELAEVLTNITFNKNQLIFIEVIMSQSDQPELLAKLGKRFGQQNS

SEQ ID NO: 30 - protein, 490 aa, Bacillus cereus
MSQYETPLFTALVEHSKRNPIQFHIPGHKKGQGMDPTFREFIGHNALAIDLINIAPLDDLHHPKGMIKEAQDLAAAAFGADHTFFSIQGTSGAIMTMVMSVCGPGDKILVPRNVHKSVMSAIIFSGAKPIFMHPEIDPKLGISHGITIQSVKKALEEHSDAKGLLVINPTYFGFAADLEQIVQLAHSYDIPVLVDEAHGVHIHFHDELPMSAMQAGADMAATSVHKLGGSLTQSSILNVKEGLVNVKHVQSIISMLTTTSTSYILLASLDVARKRLATEGTALIEQTIQLAEHVRDAINSIEHLYCPGKEMLGTDATFNYDPTKIIVSVKDLGITGHQAEVWLREQYNIEVELSDLYNILCLITLGDTESDTNTLIAALHDLAATFRNRADKGVQIQVEIPEIPVLALSPRDAFYSETEVIPFENAAGRIIADFVMVYPPGIPIFTPGEIITQENLEYIRKNLEAGLPVQGPEDMTLQTLRVIKEYKPIS

SEQ ID NO: 31 - protein, 473 aa, Bacillus cereus
MNQNRMPLYEALIEFKERGPLSFHVPGHKNGLNFPQEAIREFKDILSIDVTELAGLDDLHSPFECIDEAQQLLAEVYNTKRSYFLINGSTVGNLAMILSCCGEHDIVLVQRNCHKSIINALKLAGANPIFLDPWIDEAYNVPVGVRNEIIKKAIEKYPNAKALILTHPNYYGMGMDLEASIAFAHAHKIPVLVDEAHGAHLCLGEPFPKSALTYGADIVVHSAHKTLPAMTMGSYLHINSHLVDEEKVTTYLSMLQSSSPSYPIMASLDIARFTMARIKEEGHSEIVEFLRKFKEQLRSISQIAILEYPLQDELKVTVQTRCQLSGYELQSVFEKVGIYTEMADPYNVLFILPLQVNGGYMKVIEMIRVALQHYEVKDKRESIRYTYKGEFSLLPYTYKQLDGYETKIVSIEDAVGMIAAEMVIPYPPGIPLIMYGERITSEHKEQIMYLERAGARFQCNTKYMKVYDIESRF

SEQ ID NO: 32 - protein, 290 aa, Bacillus cereus
MRFDEAYSGKVFIKSHPSFEESKVVIYGMPMDWTVSYRPGSRFGPARIREVSIGLEEYSPYLDRELEEVKYFDAGDIPLPFGNAQRSLDMIEEYVSKLLDADKFPLGLGGEHLVSWPIFKAMAKKYPDLAIIHMDAHTDLRESYEGEPLSHSTPIRKVCDLIGPENVYSFGIRSGMKEEFEWAKEVGMNLYKFDVLEPLKEVLPKLAGRPVYVTIDIDVLDPAHAPGTGTLEAGGITSKELLDSIVAIANSNINVVGADLVEVAPVYDHSDQTPVAASKFVREMLLGWVK

SEQ ID NO: 33 - protein, 130 aa, Bacillus cereus
MDTMDTMGRHVIAELWDCDFDKLNDMPYIEQLFVDAALKAGAEVREVAFHKFAPQGVSGVVIISESHLTIHSFPEHGYASIDVYTCGDRIDPNVAAEYIAEGLNAKTRESIELPRGTGSFEIKQRETKAL

SEQ ID NO: 34 - protein, 275 aa, Bacillus cereus
MELWFTEKQTKHFGITARINRTLHTEQTEFQKLDMVETEEFGNMLILDGMVMTTEKDEFVYHEMVAHVPLFTHPNPENVLVVGGGDGGVIREVLKHPSVKKATLVEIDGKVIEYSKQYLPSIAGALDNERVEVKVGDGFLHIAESENEYDVIMVDSTEPVGPAVNLFTKGFYAGISKALKEDGIFVAQTDNPWFTPELITTVFKDVKEIFPITRLYTANIPTYPSGLWTFTIGSKKHDPLQVSEERFHEIETKYYTKELHNAAFALPKFVGDLIK

SEQ ID NO: 35 - protein, 252 aa, Bacillus cereus
MTVAQLIDIDAKKTKTSNEVYQTSTMLALLDGIYDGVISFEDLKKHGDFGIGTFDQLDGEMIAFDNGFYHLRSDGSAEKVEPEETTPFATVTFFEKEMSYTVERSMNREEVEALLHELMPSKNLFYAIRMDGTFREVRTRTVPRQEKPYTPLVEVTKSQPIFSFENTEGTLAGFWTPDYAQGIGVAGFHLHYIDDERSGGGHVFDYVVENCTIQICQKAHMHLALPETADFMAAELSRENLEDNIATAEGAE

SEQ ID NO: 36 - protein, 562 aa, Bacillus cereus
MSTGVKANDVKTKTKGADLVVDCLIKQGVTHVFGIPGAKIDSVFDVLQERGPELIVCRHEQNAAFMAAAIGRLTGKPGVCLVTSGPGTSNLATGLVTANAESDPVVALAGAVPRTDRLKRTHQSMDNAALFEPITKYSVEVEHPDNVPEALSNAFRSATSTNPGATLVSLPQDVMTAETTVESIGALSKPQLGIAPTHDITYVVEKIKSAKLPVILLGMRASTNEVTKAVRKLIADTELPVVETYQAAGAISRELEDHFFGRVGLFRNQPGDILLEEADLVISIGYDPIEYDPKFWNKLGDRTIIHLDDHQADIDHDYQPERELIGDIALTVNSIAEKLPKLVLSTKSEAVLERLRAKLSEQAEVPNRASEGVTHPLQVIRTLRSLISDDTTVTCDIGSHSIWMARCFRSYEPRRLLFSNGMQTLGVALPWAIAATLVEPGKKVVSVSGDGGFLFSAMELETAVRLNSPIVHLVWRDGTYDMVAFQQMMKYGRTSATEFGDVDLVKYAESFGALGLRVNTPDELEGVLKEALAADGPVIIDIPIDYRDNIKLSEKLLPNQLN

SEQ ID NO: 37 - protein, 169 aa, Bacillus cereus
MKRIVTATVRNQSGVLNRITGVMTRRHFNIESISVGHTESSDISRMTIVVHVESEQQVEQLIKQLHKQIDVLKVSDITEEAMIARELALIKVATSVARAELYSLIEPFRAAVIDVGKDSIVVQVTGTQDKVEALIELLRPYGLKEIARTGVTAFTRSMKKQDKQVMLIQ

SEQ ID NO: 38 - protein, 566 aa, Bacillus cereus
MSSKTEEKLATGAQLLLEALEKEGVEVIFGYPGGAVLPLYDALYDCEIPHILTRHEQGAIHAAEGYARITGHPGVVIATSGPGATNVITGLADAMIDSLPLVVFTGQVATTLIGSDAFQEADIMGLTMPVTKHNYQVRKASDLPRIIKEAFHIAKTGRPGPVVIDLPKDMVVEKGEQCSNVQMDLPGYNPNYEPNLLQINKLLKVIETAKKPLILAGAGILHAKASKELTNFARKYEIPVVHTLLGLGGFPPDDELFLGMGGMHGSYTANMALYECDLLINIGARFDDRLTGNLAYFAKGATVAHIDIDPAEIGKNVPTEIPIVASAKRALEVLLEPEGGKENHHEWITLLKGRKEQYPFSYKRNSESIKPQYAIDMLYEITKGEAIVTTDVGQHQMWAAQYYPLKNPDKWVTSGGLGTMGFGFPAAIGAQIAKPEELVIAIVGDAGFQMTLQELSVLKEHALPVKVFILNNEALGMVRQWQDEFYNQRYSHSLLPCQPDFVALANAYGIKGIRIDDPLLAKKQIQHAIELQEPVVIDCRVLQSEKVMPMVAPGKGVHQMEGVEKG

SEQ ID NO: 39 - protein, 76 aa, Bacillus cereus
MSHTFSLVIHNEPSVLLRISGIFARRGYYISSLHLNERDTSGVSEMKLTAVCTENEATLLVSQLKKLIDVLQVNKL

SEQ ID NO: 40 - protein, 571 aa, Bacillus cereus
MKQQYTAYEKLQCEEMTGAGHVIQGLKKLGVTTVFGYPGGAILPVYDALYESGLKHVLTRHEQAAIHAAEGYARASGKVGVAFATSGPGATNLVTGLADAYMDSIPLVVITGQVATPLIGKDGFQEADVVGITVPVTKHNYQVRDVNHVSRIVQEAFYIAKSGRPGPVLIDIPKDVQNAKVTSFFNEEVDIPGYKPELVPDSMKLREVAKAISKSKRPLLYIGGGVIHSGGSDELFEFARENRIPVVSTLMGLGAYPPGDPLFLGMLGMHGTYAANMAVTECDLLLALGVRFDDRVTGKLELFSPHSKKVHIDIDPSEFHKNVTVEHPIVGDVKKALHMLLHMSIYTQTDEWLQKVKAWKEEYPLSYKQKESELKPQHVINLVSELTNGEAIVTTEVGQHQMWAAHFYKARKPRTFLTSGGLGTMGFGFPAAIGAQLAKEEELVVCIAGDASFQMNIQELQTIAENNIPVKVFIINNRFLGMVRQWQEMFYENRLSESKIGSPDFVKVAEAYGVKGLRATNSTEAKQVVLEAFAHEGPVVVDFCVEEGENVFPMVLPNKGNNEMIMKRWEE

SEQ ID NO: 41 - protein, 110 aa, Bacillus cereus
MSRITHHPILGTLQSSKRITFQFNGHQYKAYEHETIAAALLANGIRTVRVHEDSGTPQGIYCNIGHCSECRMTVNNQTNVRACLTVVEENMVVESGKQHPNIVREMVKKR

SEQ ID NO: 42 - protein, 414 aa, Bacillus cereus
MSDVIIIGAGPAGLSASISCARFGLKVLVIDEFMKPGGRLLGQLHQEPTGEWWNGIKESKRLHEEAESLSVHIRCGVSVYNLDRDENNWFVHTNIGTLEAPFVLLATGAAEYSIPLPGWTLPGVMSIGAAQVMTNVHRVQVGKKGIIIGANILSFAILSELQLAGITVDHIVLPEKSELSQKAGEPEEVLNSLLNAAHLAPSAILRIGSHFMKYDWIRKAGLTFYPNSGMKINGTPLHLRKAALEIIGTDQVEGVRVANIDSKGNVINGSEKIYEADFVCIAGGLYPLAELAAVAGCPFHYISELGGHVPLHSETMETPLPGLFVAGNITGIESGKIAMAQGTVAGLSIAKYASKKRDIVDQQLYHAIQNVHSVRQKAAIQFNPMVDIGRRKMNDLWHKFQTNDKDFYKQQEII

SEQ ID NO: 43 - protein, 391 aa, Bacillus cereus
MRHCDVLIIGGGIIGCSIAYYTSKYGRDVTIIEKGEFVSGTSSRCDGNILAIDKDPGFDSQMSLVSQKLVTDLSEELEHSFEYRAPGSILVCESDEEMEAAQQWVNRQQEAGLPFRMLDRQDIREESPFFADDLLGGLECATDSTVNPYLLAFSLLSEAQKFGAKAFKQTEVKSMEIETDGSFVVETTNGTFTAQQVVNAAGVWAPKIGQMLNINIPIEPRKGHIIVASRQQHVGCRKVMEFGYLISKFGGKRKVDALTEKYGVALVFEPTESQNFLIGSSREFVGFHTRINNEVIKCIANRAIRFYPKMADMMVIRSYAGLRPWTEDHLPIISRVEHIPNYFIAAGHEGDGISLAAVTGKVIEELLNEKETIIPIEPLRLSRFTERVLNK

SEQ ID NO: 44 - protein, 137 aa, Bacillus cereus
MFQAINASGSGLTTARKWMEVTSNNIVNANTTAAPGADLYERRSVVLESNNSFASMLDGAPTNGVKIKSIEADKTENLVYDPTHPHANEEGYVRYPNIDVTAEMTNVMVAQKMYEANTSVLNANKKMLDKDLEIGRG

SEQ ID NO: 45 - protein, 190 aa, Bacillus cereus
MPTVGLNTTSTNHIPLQAGAQTKNASVNGVQSPVQQTNGVSASNQKTPGIMDKDDFLKLFLASFQHQDPFNAMDMNQMMNQTAQLSLMEQVQNMTKAVDKLQSTMYTTALDGGMKFLGKYVRGINNKGEQVTGQVETVRLAENNDVQLIVDNQVVSLRFVERVSDKPIAETNPEDEKKDDIEKNEEVKQN

SEQ ID NO: 46 - protein, 437 aa, Bacillus cereus
MIKALYTSITGMNAAQNALSVTSNNIANAQTVGYKKQKAIFDDLLYNNTVGSRGDGAYAGTNPKSIGNGVKFSGTSTDFSDGSITLTSDKMETAIEGNGLFLVGDRNSGNVEYTRKGSFGVSKDNYVTNTSGQYVLGYGVKTGTQEIDFSSRPSPIHIPMGSAVGGIQTDKATIGGNLPRNQNALSHEFTVFDEEGNSLTLRVNIKQKTTKETVDGKEVEKPVPGEYTYTVSVRNDSKNEKEFKPVEGMTGEKNLKFDTLGNLKETDEAVQKNPVTGEITKGGTVKIPFGKGLTLDLSGLTNYPTGKTISTTEVTGRPAAIANDYSISDGGFVMMRYSDGSMKVVGQLAVATFPNSGGLMKTGNGNYIATPSAGIPGIGVAGENGAGNVRGSAKESSNVDLSVEFVDLMLYQRGFQGNAKVIKVSDEVLNEVVNLIR

SEQ ID NO: 47 - protein, 432 aa, Bacillus cereus
MRLSDYNTPLSGMLAAQMGLQTTKQNLSNIHTPGYVRQMVNYGSAGGSKGYAPEQRIGYGVQTLGVDRITDEVKTKQYNDQMSQFSYYAYMNSTLSRVESMVGTTGKNSLSSLMDGFFNAFREVAKNPEQSNYYDTLIAETGKFTSQVSRLAKNLDTVEAQTTEDIEAHVNEFNRLAASLAEANKKIGQAGTQVPNQLLDERDRIMTEMSKYADIEVSYEATNPNIASVRMNGVLTVNGQDTYPLQLQKDKKPMSVQISGTDIPLSGGTILSAIDTKAKITNYKDNLNEFVNSLKKQVNNTMKKELFVGEDAKKLELNPDFIKDISKMKISAETANNLAAITDKGYKDGLTYKQALDQFLVGVASDKSSVNAYQNIHKDLLEGIQQEKMSIEGVNMEEEMVNLMAFQKYFVANSKAITTMNEVFDSLFSIIR

SEQ ID NO: 48 - protein, 287 aa, Bacillus cereus
MRVSTFQNANWAKNQLMDLNVQQQYHRNQVTSGKKNLLMSEDPLAASKSFAIQHSLANMEQMQKDIADSKNVLTQTENTLQGVLKSLTRADQLTVQALNGTNSEKELQAIGVEIDQILKQVVYLANTKEQGRYIFGGDSAKNPPFTEDGTYQGGKNDVNWKLNDGYEFKAFRNGGALLSPVIKTLKQMSEAMKNGDQKALKPLLEGNKQNLDGIINRTTEVGSTMNTMETFKTILNEQNVALQENRKEIEDVDLAVAISDLAYINATYEATLKAVSTMSKTSILDYM

SEQ ID NO: 49 - protein, 688 aa, Bacillus cereus
MFKIESARTYFSIFLAASFVVALLIPLPPFILDIIIVFLLSMSVLIYMRATSINEWDELKSFPTMLLLIGIFRVSINVSTTRAILTDGNAGHVIEEFGQFVIGGNLLIGIVIFTVLIIFQFIVANGASRTAEVAARFTLDSLPGKQMSIDADLNQRIISEKDAQAKRKKLNMETEFYGAMDGAGKFIKGDVIFGIVILFVNIIFGLIVGMMQQGMSFADAALHYTQLTVGDGIVNQIGSLMLAISTGIIVTRVFDGSPDTVTEGIFKELLAHEVVVYALGGLFIAMGIFTPLPFLPFALVGGTIIFLGIRNKNRIKKEKEDELQKEIEMIQGEDEQLQQVEDSFGVFTDKYPIIVELGLDLAALVKQKINGETARDKVVLMRKSIITDLGINVPGINFKDNTSFRPRGRYIIRIKGAKAAEGVLKSGYLLALKTPNVMADLDAEPAKDPIFGEDGYWILEHMVQDAQMKGYQVLEPLSILITHLDVVVRRNLHELIQRQHVKDLINSLENDNGVLLEEIKKKEIDLSLVQNVIKQLLKEGISIRDLPTIIEGIIDGKEIYQNHVDGVTSFVRECISKVICENAKNPDGKIYAALFSDSIELDADVVNNSYQGYLLNWDLDLETRVVEQVQRVFKQARLMGREPVLLTRRKDFRFAIVRLLERYQVEAQVLCISELAPEIVVDQIAYIE

SEQ ID NO: 50 - protein, 348 aa, Bacillus cereus
MAKDNKTEKATPQKRKKSREEGNIARSKDLNNLFSILVLAVVVYFFGDWLGFEIANSVSVLFNQIGKNTDSTEYFYLMGILLLKVSAPILILVYAFHLFNYMIQVGFLFSSKVIKPKASRINPKNYFTRLFSRKSLVDILKSLFYMGLIGYVAYVLFKKNLEKIVSMIGFNWTASLTEIIRQIKFIFLAILIILIVLSIIDFIYQKWEYEQDIKMKKEEVKQEHKDNEGDPQVKGKRKNFMHAILQGTIAKKMDGATFIVNNPTHISVVLRYNKHVDAAPIVVAKGEDELALYIRTLAREQEIPMVENRPLARSLYYQVEEDETIPEDLYVAVIEVMRYLIQTNELEV

SEQ ID NO: 51 - protein, 266 aa, Bacillus cereus
MRIGTNVLNMNARQSLYENEKRMNVAMEHLATGKKLNHASDNPANIAIVTRMHARASGMRVAIRNNKDAISMLRTAEAALQTVTNILQHMRDLAVQSSNGTNSNKNRNSLHKEFQSLTEEIGYIVETTEFNDLSVFDGQNRPITLDDNGHTINMMKHIPPSPTQHDIKISTEQEARAAILKIEEALQSVSLHRADLGAMINRLQFNIENLNNQSMALTDAASRIEDAEMAQEMSDFLKFKLLTEVAICMVSQANQIPQMVSKLLQS

SEQ ID NO: 52 - protein, 287 aa, Bacillus cereus
MRINTNINSMRTQEYMRQNQDKMNTSMNRLSSGKSINSAADDAAGLAIATRMRAKEGGLNVGARNTQDAMSALRTTDSALNSISNILLRMRDLATQSANGTNDGKDKDSLNLEFKELQEEINHIAGKTNFNGKNLLAAAGTDKNISIQLSDVASDNLTITAVDATTGATGLGLTKTIGAPAAPVIPAPPAPAADNDAAGAIVQLDAAIQKVADMRATFGSQLNRLDHNLNNVNSQATNMAASASQIEDADMAKEMSEMTKFKILNEAGISMLSQANQTPQMVSKLLQ

SEQ ID NO: 53 - protein, 274 aa, Bacillus cereus
MRINTNINSMRTQEYMRQNQDKMNTSMNRLSSGKSINSAADDAAGLAIATRMRAKEGGLNVGARNTQDAMSALRTTDSALNSISNILLRMRDLATQSANGTNDGKDQDSLNLEFKELQGEIDHIAGKTNFNGKSLLAAKGTDIDIQLSDISGDKLTIASVDATTGADGLNLTKTIASTGKGGDAAESIKELDKAIQSVADMRATFGSQLNRLDHNLNNVNSQATNMAASASQIEDADMAKEMSNMTKFKILNEAGISMLSQANQTPQMVSKLLQ

SEQ ID NO: 54 - protein, 271 aa, Bacillus cereus
MRINTNINSMRTQEYMRQNQDKMNTSMNRLSSGKSINSAADDAAGLAIATRMRAKEGGLNVGARNTQDAMSALRTTDSALNSISNILLRMRDIATQSANGTNETKDQASLDLEFQELHKEIDHIAEKTNFNGKNLLAATGADIDIQLSDVSGDKLTIASINAKADTLLTATGTNVKTTADASTAITNLDKAIQTVADNRATFGSQLNRLDHNLNNVNSQSTNMAAAASQIEDADMAKEMSNMTKFKILNEAGISMLSQANQTPQMVSKLLQ

SEQ ID NO: 55 - protein, 271 aa, Bacillus cereus
MRINTNINSMRTQEYMRQNQDKMNTAMNRLSSGKSINSAADDAAGLAIATRMRAKEGGLNVGARNTQDAMSALRTTDSALNSISNILLRMRDIATQSANGTNDTKDQDSLDLEFQELKGEITHIAEKTNFNGKTLLAGVAAANNIDVQLSDVSGDKLTITAIDATAATLKITGDVKSTKNASDSITALDAAIQTVADNRATFGSQLNRLDHNLNNVNSQSTNMAAAASQIEDADMAKEMSNMTKFKILNEAGISMLSQANQTPQMVSKLLQ

SEQ ID NO: 56 - protein, 458 aa, Bacillus cereus
MAGTISNYGDRQQIWNLGNNIIDTKKLVDLELQALEMKKSPYTTQKQTLTNENKVYASMKKEFANFVQVFKDLNTFKGDEKKTTLSKDGFMTAQADAAAIPGTYTITVERVAERHQITTAPLTPPKTPEGTEQKFSLDLKLGVDDVFQINGKEVKISKDMTYKDLVNKINNGNYGASVYTLGDQLFFTSTTAGEAGELKLTDGANGFLQNIGLVTSAKNPDGTNVVAHQVTGAINAEYTINGIKGTSKTNKIDTIPGLTINLEKVTTEPIKLTIEDSDIKNSIDLIKKMKDEYNNAVKSLDLFSGENGVMQGNNVSFAISNAMTSIFKFSQDDKYLFSFGIQIDKTGNMTLDEEKLKIAFKENPESTKQFFFGFNGIGHDIDKKLEGIFGDEGIIGKRSKSIEKQVTDLERKIQDIDTINKKKQESIIDKYAKLESQLALLDSQLQTIKAMTKTKSDD

SEQ ID NO: 57 - protein, 78 aa, Bacillus cereus
MAQISQRAGKATDTSLDNYTLGKRMKDIDSRITNFERRLELTEARYWRQFSEMERAISMMNQQSSMLMSNFGSGMAQG

SEQ ID NO: 58 - protein, 99 aa, Bacillus cereus
MKIQPMLHTQPFGAIQSIGAPKTSQTSVVEGKKFIDLLEDMNQTQNNAQTAVYDLLTKGVGETHDVLIQQKKAESQMKTAALVRDNLIENYKSLINMQI

SEQ ID NO: 59 - protein, 524 aa, Bacillus cereus
MEKMKNVIQSLKTWHKLVIGAALLAIVTGALLYFTLPDKYVVVYQNLNDADKQEITAELSKLGVDYQLAADGSIRVQKNDAPWVRKEMNGMGLPFNSKSGEEILLESSLGSSEQDKKMKQIVGTKKQLEQDIVRNFATVETANVQITLPEKETIFDEEKAKGTAAITVGVKRGQLLTADQVAGIQQMISAAVPGVKAEEVSVIDSKKGVISKGADEAHSSSSSSYEKEVEMQHQLEGKLKQDIDATLMTMFKPNEYKVNTKVSVNYDEVTRQSEKYGDKGVLRSKQEQEESSTAQEGADTKQGAGITANGEVPNYGTNNNQNGKVVYDNKNGNKIENYEIDKTVETIKKHPELTKTNVVVWVDNDTLVKRKIDMTTFKEAIGTAAGLQADPNGNFINGQVNVVTVQFDQPKAEKEKEPEKSGMNWWLFGGITAGLLAIGGLVWFLLARRKRKKEEEEYEEYLAEEEIAASNESILEIPEEKIVPEPKPEPEEPKEPTLDEQVQDATKEHVEGTAKVIKKWLNGQ

SEQ ID NO: 60 - protein, 334 aa, Bacillus cereus
MLDEISSKEKAAILIRTLEEGVAAKVIEYMTAEEKEVLLREIAKFRVYKPETLENVLGEFLYELNVKELNLVTPDKEYIRRIFKNMPEDELEKLLEDLWYNKDNPFEFLNSLTDLEPLLTVLNDESPQTIAIIASYIKPQLASQLIERLPDHKRVETVMGIAKLEQVDGELINQIGELLKSKLNNMAFSAINKTDGLKTIVNILNNVSRGVEKTVFQKLDEVDYELSEKIKENMFVFEDLLGLEDLALRRVLEEITDNGVLAKALKIAKEEIKEKLFTCMSSNRKEMILEELDGLGPLKMTDAEKAQQTITGTVKKLEKEGRIIVQRGEEDVLI

SEQ ID NO: 61 - protein, 434 aa, Bacillus cereus
MSRLLMNENEKWNKFIETPLYTKVGKVHSVQEQFFVAKGPKAKIGDVCFVGEHNVLCEVIAIEKENNMLLPFEQTEKVCYGDSVTLVSEDVVVPRGNHLLGKVLSANGEVLNEEAENIPLQKIKLDAPPIHAFEREEITDVFGTGIKSIDSMLTIGIGQKIGIFAGSGVGKSTLLGMIAKNAKADINVISLVGERGREVKDFIRKELGEEGMRKSVVVVATSDESHLMQLRAAKLATSIAEYFRDQGNNVLLMMDSVTRFADARRSVDIAVKELPIGGKTLLMESYMKKLLERSGKTQKGSITGIYTVLVDGDDLNGPVPDLARGILDGHIVLKRELATLSHYPAISVLDSVSRIMEEIVSPHHWQLANDVRKILSIYKENELYFKLGTIQQNEENAYIFECKNKVEGINTFLKQGRSDSFQFDDIVEAIQHIV

SEQ ID NO: 62 - protein, 328 aa, Bacillus cereus
MSGEKLSQEQIDALLKAVNEGEEMPAFAQEAGKQEKFQEYDFNRPEKFGVEHLRSLQAIASTFGKQTSQTLSARMRIPIELEPSTVEQVPFTSEYVEKMPKDYYLYCVIDLGLPELGEIVIEIDLAFVIYIHECWLGGDSKRNFTMRRPLTAFEFLTLDNIFLLLCKNLEQSFESVVAIEPKFVTTETDPNALKITTASDIISLLNVNMKTDFWNTTVRIGIPFLSVEEIMDKLTSENIVEHSSDKRKKYTSEVEVKVNQVYKPVHVAIGEQKMTMSEIEQIEEGDIIPLHTKVSDELLGYVDGKHKFNCFIGKDGTRKALLFKSFVE

SEQ ID NO: 63 - protein, 252 aa, Bacillus cereus
MRIKKQLSLLAVIFVFSIVFSIIFVNPAYAAPNGFINFENGKEFTSNSSVQLFALVTLLSLSSSIVLLFTHFTYFMIVLGITRQGLGVMNLPPNQVLVGLALFLSLFTMQPVLGQLKSDVWDPMTKEKITVSQAAETTAPIMKEYMSKHTYKHDLKMMLKVRGEELPKDLKDLSLFTLVPSFTLTQIQKGLLTGMFIYLAFVFIDLIISTLLMYLGMMMVPPMILSLPFKILVFVYLGGYTKIVDIMFKTVA

SEQ ID NO: 64 - protein, 91 aa, Bacillus cereus
MNTSPIIDIFQTFFYKGVMILMPVAGVSMIVVIIIAVIMAMMQIQEQTLTFLPKMASIVLVIIILGPWMFQELTTLILDLFDKIPSLLRSY

SEQ ID NO: 65 - protein, 253 aa, Bacillus cereus
MNMELWAATFFAFCRITSFLYFLPFFSGRSIPAMAKVTVGLALSITVADQVDVSHIKTVWDVAAYAGTQIVIGLSLSKIVEMLWNIPKMAGHILDFDIGLSQASLFDVNAGSQSTLLSTIFDIFFLIIFISLGGINYFVATILKSFQYTEAISKLLTTSFLDSLLATLLFAITSAVEIALPLMGSLFIINFVLILIAKNAPQLNVFMNAYVIKITCGILFIAMSVPMLGYVFKNMTDVLLEEYTKLFNFFLTK

SEQ ID NO: 66 - protein, 122 aa, Bacillus cereus
MQAWQRYMQNDIMTSNPIKNTIFIYERCIVEFRKLEELLNTFKLQEGDDLLEKLERIFEELKLQLNPDISKDLYDSLFGLYDWISIQIQTMKVTREAKDIDAIVQVLQDLIDGYRGALENEQ

SEQ ID NO: 67 - protein, 264 aa, Bacillus cereus
MDFATIIGLILGFVAVVVGMVVKGADITALLNPAAALIIFIGTFAAVCIAFPMNQLKRVPKLFKVLFGSNKKDLSYEQLLELFVHWTSESRKYGILSLEQQLDKIQDEFLLRGMKFVIDGVSAEDLEQILESELEAIEERHAKGAAIFSQAGTYAPTLGVLGAVIGLVAALGNLTDIEKLGHAISGAFIATIFGIFSGYVLWHPFANKLKQKSSAEIEKKRLIIDCLLMLQEGTYPFIMKNRILGALSATERKKLEKGAEKNAE

SEQ ID NO: 68 - protein, 262 aa, Bacillus cereus
MRSKKNRRGKKKKHDEHIDETWLIPYSDMLTLLFALFIVLFAMSSIDAAKFKQMAVAFRSELAGGTGNKEFLSDQKPNDEKELSASSLEAEQTKKQEEARAKEKKEMDELKALQKKIDQYINEKQLSSSFQTKLTEKGLMVTILENILFDSGKADVKLESLGIAKEMSSLLVSASPREITVSGHTDNVPIANAQFASNWELSTQRAVNFMQVLLQNKELQPEKFSAIGYGEYRSIAPNDTQEGKAKNRRVEVFILPLTEKVK

SEQ ID NO: 69 - protein, 277 aa, Bacillus cereus
MGEKNQVLARPQRRKRKFDISSPVGIIVGFIIVIAAIMLGGGGIKAFKNFLDISSILIVIGGTTATIVVAYRFGEIKKYTKSIFTVLHRREEDLEQLTDLFVDFSKKSKKNGLLSLEVDGEQVDNPFIQKGIRLMLSGYDEDELKEVLLKDIETEVYELRKGAALLDKIGDFAPAWGMIGTLIGLIIMLQNLQDTSQIGTGMAVAMLTTLYGSVLANMIAIPLAEKVYRGIEDLYTEKKFVIEAISELYRGQIPSKLKLKLDTYVYETKVKKVKGAA

SEQ ID NO: 70 - protein, 225 aa, Bacillus cereus
MSKGPQKGSPRWMTTFTDLTMLLLTFFVLLVATSKQDAVKLSKMLEKFSDTGQVDAKVMENTIPDISHEKNDEKMISKKRMDELYKKLKAYVDNNGISQVNVYREDTGVSVVIVDNLIFDTGDANVKPEAKGIISQLVGFFQSVPNPIVVEGHTDSRPIHNEKFPSNWELSSARAANMIHHLIEVYNVDDKRLAAVGYADTKPIVPNDSPQNWEKNRRVVIYIKE

SEQ ID NO: 71 - protein, 672 aa, Bacillus cereus
MQTDLLNIFFEESEEHLQSLNENVLVLEQNPADMDVVGEIFRSAHTFKGMSASMEFTEMADLTHKMENVLDEIRHGNIVVNADIIDVIFECIDNLEKMVADVQQGGMGNIDVASTKQKLEALLNGNVETPTEHIEQNHIDTDDAVSHEVHITVEQQAILKAVRAIMCIEALQNVGNIQKTAPSIEEIEADAFGFEFTVFMDTDCSIEELKQVVLHVSEIEKVEVKQGEPISKEVASKKVVTQEVVQVEEKLQPAVVTQVNSPIEATNQPSSTMPAKSTTKTKNAKVENRSIRVQLEKIERLMNMFEESVIERGRIDELAQTIQNKELIEHLNRLGDISKDIQNVLLNMRMVPIETVFNRFPRMVRMLAKDLGKKIDLQITGEDTEVDKIVIDEIGDPLVHLIRNAIDHGVETVEKRRDAGKNETGTIKLEAFHSGNHVVIQITDDGNGINKGKVLEKAIKNGVVTEADANRLTDREVFDLIFQPGFSTAEVVSDLSGRGVGLDVVKHTIHSLGGHLIIDSEEGKGSTFRIELPLTLSIIQSMLVQTNDKRYALPLGNIVEAIRIKREDIQSLQGKDVLNYRNQIIEVKHLSTVFGEKTVDEAFASYDGQMVPVLIVRNTHRSYGLIVNTIIGQREIVLKSLGDFFAESSNYFSGATILGDGRVVLILNPEGL

SEQ ID NO: 72 - protein, 285 aa, Bacillus cereus
MENKYYNFDPSVDTDERTNLEIELLLEAVFKLSGFDFRQYARTSIYRRICNRMQLSNIPTISKLIEKVIHEEGVLEQLLNDFSINVTEMFRNPAFFKALREHVIPELKKQPEIRIWHAGCATGEEVLSMSILLHEEGLSEKSVIYATDMNTNVLEKAKQAILPLNKMQTYTKNYLQAGGTQAFSNYYSTDNRFAYFNPSLLQNIIFAQHNLVTDQSFNEFHIILCRNVLIYFTSKLQNQVQHLFYESLSHNGFLCLGNKETLRFSNIMPHYTQFNPSEQIYQKIQ

SEQ ID NO: 73 - protein, 265 aa, Bacillus cereus
MPIVNMIIEQDYDHFIASFKQQFNMDIASYKQDRMRRRIDAFISRKGFENYTNFLNKLRADQNLFLNFIDYITINVSEFFRNKERWQTLESKALPKLLEQNSGKLKVWSAACAAGEEPYTLSLILSKHLAPFRFEIQATDLDFHILETAKRAQYTERSLKELPTDLKERHFTKENGLYSLHQNIKQNVSFKQHDLLMQSFDTNYDLIICRNVMIYFTEEARIKLYEKFSRSLRKGGVLFVGSTEQILTPERYNLQRFDTFFYEKI

SEQ ID NO: 74 - protein, 122 aa, Bacillus cereus
MAHKILVVDDAMFMRTMIKNLLKSNAEFEVIGEAENGVEAIQKYKELQPDIVTLDITMPEMDGLEALKEIIKIDSSAKVVICSAMGQQGMVLDAIKGGAKDFIVKPFQADRVIEALTKVANS

SEQ ID NO: 75 - protein, 302 aa, Bacillus cereus
MSQAQSILLESGTNELEIVTYTVGENLFSINVMKVREIINPFPVTTVPESHHAVEGVVQVRGEILPVINLAMALNLKSTKPLDQTKFIISELNQMKVIFRVDEVHRIQRISWEQIDEPASLSMGLEETTSGIVKLDGKIILLLDYEKIVCEISGTGYDNKSIAGLEQKTDRAEKVIYIAEDSAMLRQILEETLSSAGYTKMNFFSNGAEALAQIEKLAKEQGEKMFEHIHLLITDIEMPKMDGHHLTKVVKDSEVMNRLPVIIFSSLITNELFHKGEAVGANAQVSKPDIQELIGLVDKLVL

SEQ ID NO: 76 - DNA, 1095 bp, Bacillus cereus
  source 1..1095: mol_type = genomic DNA
atgattatcaatcacaacattacagcacttaacacacacaacaaactttcgagcgcatcttctgctcaaagtaaatcgatggagaaattagcttcaggtcttcgcatcaataaagcaggcgacgacgctgctggtcttgcgatttctgaaaaaatgcgtgcacaggttcgtggacttgaccaagcttcacgtaacgcacaagacggaatctcaatgattcaaacagctgaaggtgccttaaacgaaacacacgatattcttcaacgtatgcgtgaattagcagttcagggtgcaaacgatacgaacgttactcaagaccgcgacgctattcaagaagaattaacagcattaaagagcgaaatcgaccgtatcggtgaaacaacagaattcaacaaacagacacttttaaatggtggacttggtggaactgttgatcaagatgttgcaactactacagttttaggtgttacaggtgtagcaggtgcttctactaacggtgctgtcgctggatcatatgcaatcactagcggaactgctggcgaattgacaatgacattcggaactaaaacgcaaacaatcagcaacgcaaacggcgctcaagacttgaacttctctgagttcggtatctcgatcaaaacaaacgctggttacactgcagatgacgcagttggtaacgttgttgtagatcctggagcagttacattccaaatcggtgctaacgaagaccaaaacttgagcttgaacatccgtaacatgaaaacagacggcgtattaaacctcgcaactgcaggacgtgtagatgtttcgactcacgcaacagctaaagcttcagtaacaaacatcgaaggtgctatcactgaagtatctaaagaacgttcgaaactcggtgcttaccaaaaccgtctcgatcacacaatcaacaaccttaaaacttcttctgaaaacctaacagcggctgaatcacgcgttcgtgacgttgatatggctaaagaaatgatgaaccaaacgaaaaactcaatccttgcacaggctgcacaagcaatgttggcgcaagcaaaccaacaaccgcaaggcgttcttcaattacttcgttaa

SEQ ID NO: 77 - DNA, 942 bp, Bacillus cereus
  source 1..942: mol_type = genomic DNA
atgttaacaaaaacgaatatcggacatttatcagcgagttatcaaaagctgagctcgatgcaggaacaactgatcagcggtaaaaaaattcagcgcccgtccgaagatccggtcgttgcgatgcaaggcatccgttaccggacagaagtccgggaagtggaacagttcaagaaaaacgtcaatgaagcgacagggtggatggatttgaccgattcagctctcaatgaagtgacatccgcgatgagccgagtccgtgaattgacgacacaagccgcaacggatacatatgatgcgacccagcgaaaagccatccaaagtgaagttggtcaattgattgagcatattggtacgctcgctaatacgaaatacaatgaaaaagcgatttttaatgggactaaaacagatcaacccttcatttcaatggaaaatctgaaggactatttaacgacatccggtaaatcggttgataccgtctttaccgatggaaaccctgtaacaaaagagaatgaagtgattcgctatgaaatatcgtccggcattgaagttcaagtcaatgtttcgccaacaaatgtgtttagtacagaaactttcatgacgctaaaaaaagtgtatgatgccttaggtggtacttcggacgcaggtcccgtcgacagttccaacaatggagctgaactatcgggtatgttgaaagatcttgatagcatgcttaatcagacagttgaaacacgagccgatctcggggcgcgggtcaatcgacttgagctgaacgcttcacgccttgaagatcaagaaatcatcgcgaaatccgtcatgtcggacaatgaagatattgaggctgaaaaggtcatcatggaattgaagtcatacgaaacgctacaccgcgcggcgcttagtgcaggggcgcggatcattcaaccgactttgctcgatttcttacgttaa

SEQ ID NO: 78 - DNA, 1530 bp, Bacillus cereus
  source 1..1530: mol_type = genomic DNA
atgggatcaacgttcatgggacttgagaccggacgacgtgcactgacgaccaatcagtgggcgctccagtcgacgggaaacaatatcgcaaatgcagggacagttggtttttcgcgtcaacgactgattatggcgacgacagaacaacttacaatggcaatcggaactggcaagatggggcaaattggaactggtgtaaaaggtgagatgcttgaacgtgtccgcgacgtcatgctcgacaaacaataccgtgatgaagcaacgaagacgtcttactatggcacgaaagaagcggcatttagtcggatggaagacgttatcaatgagccatcggatacaggactgtccaaagcgtttgatggtttttgggaatcattgcagacattgtcgacgaacccgcaagactctggtgcccgcagtgttgttcgtcaaaaagcagagacgttgacgcagacgttcaactatatggcgaaggcgcttaatcaagtacaaggggacttgaaaagtgaaattgaggtctcaaccaaaaaagtgaatgacttgttcaaaaaaattcataacatcaatgccgagattcataccgtcgagccgcttggtgttctgccaaatgccttgtatgatgagcgcgaccgctattttgatgaactgtcagagtatgtcgattttgaaaaagtgtctgtcgatggcgatgcgattcaacaaggaacacttggaaacaccgtcaaaacggctgaaggccgaattgacgtccgtatcaaactccctaacggggataaactgctggcagtggattcagatttaccacaagcaggaacgctgacgttcacaacggacgataaaggtttgtatacaggatttaaaaccgattcacaaacgatttcattcgacgcggctggtggtttttcatcaggacgtctgattggtttaattgagatgtacggacatgtcgaaaatggtcaagcagcaggcgaatatgtcaaaatgcaaggacatcttgatgagatggcggcaacctttgcaactgcttttaacgaggcacatgctacgaatgtaaaaaaagataagacggcaggtaccaatgaatttttcgtttcttcaaatggtgggaccattacagcaaattcaattacacttggaacggatataaaaaaaagtctggataatattgcaacttcgactgacggcaatattggtgatagtgccggtgccttgaaacttgccaacatgaagaccgcaagcatccgttttgaacgatcggatacgacgacgacgatcggttcgttttatcaaaacgttattggagatatggcggtggcaacagatcaagtcgcacggctcggtcagagttcggcagtcttgatggaaagtgcggaacaacgccgtatgtccgtctccgctgtttcaattgatgaagagatgacaatgatgattcagtatcaacatgcatacaacgcggcagcgcgtaatatcacgacggttgatgaaatgctcgataaaatcatcaacggtatgggaatcgtaggacggtga

SEQ ID NO: 79 - DNA, 408 bp, Bacillus cereus
  source 1..408: mol_type = genomic DNA
gtggaactcatcaaccagctcacgacaacgcatatggatctgctggaactggcacacgaaaagaaacaggtcctgattcaaaacgatatgccacgcctgtcgcaaatcgtcaaggaagaactggtttatctgaaacggatggagcagttggaacagcaacggattgaacacatgggagcggtgacaatgacggaatggcttcaagtccatccggaagatgtcgaggccatgcgccagttacttcaggcaatcggtaagctcaaaattatcaatgaattgaacgccgacttgctcgaacaatcgttacaatacctcaactggcatctcgaactattagtgccagaagcagatgattttacatacggtcaatcggcgcttgatcgcgcccacttcaatcgaaacgcctaa

SEQ ID NO: 80 - DNA, 249 bp, Bacillus cereus
  source 1..249: mol_type = genomic DNA
atgcgaattgattctacgaaatgggttaacatgcctaagacgtacgaaagaaatcaaaaagtagaaggaacagaagcaacacgtacgaatcggccggacgaggtgacgatctcaagcgaagcgcggatgcgtttcagtgagacaggcacatcccggaccgagaagattgaatcccttcgccaagccattcaggatggaacctataaaccggatgcgaaaaaaatcgcagaacgtttcttgaacttgtaa

SEQ ID NO: 81 - DNA, 1359 bp, Bacillus cereus
  source 1..1359: mol_type = genomic DNA
atgaagttgaaacgaatcatcccgattatggcattatcgacattatcccttagcacgatgatctcgacagataatgccgaggccaagacgaaatcatccaaatctccggaaatccgtaacgtcatctttttgatcggtgacggaatgggtgtttcttatacatctgctcaccgttatctgaaaaacaatcccgctacacctgtcgctgagaaaaccgcattcgatcaatatctagtcggtcaacagatgacctatccggaagatccggaacaaaacgtcaccgactctgcttctgccgcaacggcgatgtcatccggtgtcaaaacgtataacgcggcaatcgctgtcgacaacgataagtcggaagtcaagaccgtccttgaagcggcaaaacaacgcggcaaatcgacgggactcgtcgcgacgtccgaaatcacacacgcgacgccggcctcattcggggcgcatgacgagaaccgcaagaatatgaacgccatcgccgacgactatttcaaagaacgtgtcaacggaaaacacaagattgacgttctgctcggcggcgggaaatcgaacttcgtccgtccggatgttgatttgacgaaatcgtttaagaaagacggttacagctacgtcacggatcttgatcaaatgcaggcagataagaacaagcaggtacttggtctgttcgcggacggcggactgccaaaacgaatcgaccgcgagaataccgtcccgtctctcgagcagatgacgaactcggccatcaaacgtcttgattcgaacaaaaaaggcttcttcttgatggttgaaggaagccaaatcgactgggcaggtcacgataacgacatcgtcggagcgatgagcgagatggaagacttcgaacgtgccttcaaagcagcgatcgctttcgccaaaaaagataaacacacactcgtcgtcgcaacagccgaccattcgaccggtggttactcgatcggagcagacgggatctacaactggttcgccgagccgattaaagcagcgaaaaagacgccggacttcatggcggcaaaaatcattgaaggtgcagatgtccggaagaccttgacgacgtacatcgatcaaaaccgacttgccttgacggaggaagaaatccagtccgtttcacgtgccgcggagtcgaagaaagtgctcgacgtcgataatgcgatcgaagacatcttcaataaacgttcgcacaccggctggacgacaggtggacacaccggggaagatgttccggtctatgccttcggtcctgcaaaggaacgattcgccggacaagttgataatacggatcacgccaaaatcatcttcgatctgttgaaatcgaagaaataa

SEQ ID NO: 82 - DNA, 2976 bp, Bacillus cereus
  source 1..2976: mol_type = genomic DNA
atgtcaatcacttcactccgatcacccttattggaaaccctaactaaaatcacaaaacagactccgatgaaagaagtattactgacggaaggcgctacgtataccttagaagatatccgggtgcgttccaatgcgatggcacaccaactagagaaatcatttacgacacaaagatatatcccggtttatacgaccgacaacgtccaatcgatcttcagcatgttggcgtgttggaaagcaggaaaagtctacgttccattgaacccgcagacccctgtgccaaaaatccaccaactaatgtccacactccacagtgagtcgatttggacagatggactaccggaagaatacgatatcccgcagatcatcttttctaaagaacgaatggaacatgatgttgacgtggtgggaacagaggtcgcttatatcctaatgacatctggtagcacgggcgaacctaagaaggtagaagtaacgcacgccaacttggattggttactgcgtacattggagcagaccatcccatttgcaaagaacgatcgttttctcgtctcgacgccacctgcttttgatgtcgtccttcatgaaatgttggcgttcctctatggagagggacaagtcgtctgtttcccggcatattcgaacatccaaaagattaaggaattgccaaacttcgtcaagcgttatgacattacgcatatcgcgttatcaccatctgcctgtacgcaaatcttgaaccgtgaaaacgaacgcgtgaagttggcttctttacgaaaagtattattagcgggagaggcgctaggagtaccgctcgtccaaaaacttcatacacatttcccgaacgtcgacgtctacaatctatatggaccgactgagacgacagtctatgcgacttgcgtgaagattgatgatgtagtaaatgaagagatacctatcggaaaagcacttgccggtacttctattatatttcgtgataaagacggacagttgaacaattccgctggtgagatattgattggcgggaacggcgtaagtcgtggatacctgggcaatccatcattgacagaagagaagtttctgaccattggagaggcacgctattacgctacaggtgaccatggacgaaaggacgagagcggcataattttttacgagggaagacaagatgatcaagtgcaggtgaacggaattcgggtcgaactcggagaaatcaatgcagcactccatgcggttcgtccgacaggtaccttcgaaacattatatctagcgaatcgcctagtcgtctttagcgatactcattcgttcacgtcagaagatgcgcaaatgttgaaagaagaactgaagaaacgaatcccttcgtatatgattccgagcatgtatgtgaccgtaccagaattcaaaatgacggccaaccgtaagctggaccgtcgttatctcgagtctttcatcgagacgacggatataggagagctcgttaaggaggaatcagttcaaaatatggatcaacttttggtgcgtgtttcgaatcactacggtcgtccagtcacaccagatatggatctcatacacgatcttcacttggactcgcttgatcagctcgatcttctcctcttgttggaggatcatttcaatatgacgcttgtagatgattttgtagtgacgcatccaacgcttcgccgggttcagatacaattgtcacgagaggaagagagagggacgaatatcatcgaggttgatgaagaatattggaacggcgtcgtcgaagacaatatggtggcgaaccgggcgcgatatgaacaagtgacaaaagaacaaaaagaaacgttttatctgcaaaagagttatcatgtcgatggtttccgacaagtgttacacgaagttgtctcgatacctgagacatatgaacggagccaattgcaggcagctgtcgatgccttgattgtacgccatccaatgttacggagcttcttgaaggtgcaagaggatcgtttacagttcacagtgtattcagaaaaggcgtcattccgcttgttatctgtgccgacagttactgaggatcaacgacagaactggattgaaagaatgaaacaacagtacttagaggacttgatggcatatttcattcatgatgtgtctacgaaccgtatcgaactatttatcaaccaccatgtcgcagatcaggcatcgatgaacctcttgaagcaggatctatctgccttgcttcagggcaaggtattatgtcctttagctgtcgactattgggattacatcgattacatcgatgcgaatattgatcgagcggagtcagaaatcgagcaagtgagtcattccggattcgccgacgtcacaagcgacgcgtttgacgtcaacgaaggtcgtccggttcgatatctatcgttcctgctcaagcgagaaacaccggaagagtatattgcttatgccaattacatcatcttaggtgccttggcagccggacaatcacgacaacagatgagtggttcgactatcgtcgatctacgatcgtttaacggactcgacattaagggagtcgttggagacgttcatactacaatcccactctgtcttgagcagggagagtcgttcgagacattcaaccggaagttcgacagctggtataaacagttcgagtctggaattaattttaatcatctactgtatcgcgactatccacatgtccagagtgcacatcgtacgttcgagcataacttggatgacaatctgaaagtaagctcgagcttcctcggtgcaatacgagaacaagagattccgacgatgttagaagaactaaaagcttcacataccattcttcagaatttctcgacacgtaagctctatgcttccatcttctatacaggaaagcagttaatcatcgtcccactcagtcaaccaatgttatcggaagactttattactagactaggtggtgagatccatcatgaagacgaaccgaaataa

SEQ ID NO: 83 - DNA, 777 bp, Bacillus cereus
  source 1..777: mol_type = genomic DNA
atgaaacggaaaaagaagccgcatgacgaacatatctccgaaggttggctgattccttatgccgatcttctgacgttattactggccttgttcatcgttctgttcgcttcgagtaacgtcgatgccgttaagcttaaagcaatgtcccaatcattcagctccgtctttaacggcggttccggaatgatcacgaacagttcattgtcgacctcacaagaagaagaagattcaaaaaaaacggatgcccgaacaaaggcgcaaagttatgaaatcgctgaacttgaaaaaatcaaggaagaagcaaatgactacatcaaacaacagaagcttgaaaaagacatcaaagtcgaagtgacgaatgaaggactggtcttcacgatccgcgaccgtgcgctcttttcccctgcccaggccgaagtgcgtggcaatgccgttcagattgcccaggggatgagtaatttactcgttaaagccggtcagcgccaaattcaggtgtccggtcatacggacaacattccgatcaacacggcacaatatccgtccaactgggagctgagcactgagcgggcgatcagtttcatgcgggcactccaacgtaattcgcagcttgctcctaaacggtttaccgttagtggatacggtgaatatcaaccgatcgcttccaaccagacggaaagcggtcggagtcaaaaccgccgtgtcgaagtattgatccgtccgttgattgacatcaaagcacaaaatgttctcgacgagacgaaagtcacaccatcataa

SEQ ID NO: 84 - DNA, 786 bp, Bacillus cereus
  source 1..786: mol_type = genomic DNA
atggatatcgtgtcgattattggtattatactaggcctcatcaccctagtcggaggaatgattttaaaaggggcttcgccggtcgccctcttgaacccggcggcacttgtcatcatctttgccggaaccattgcggccatcatgatttcatttccgaaagagcgactcaaaatcgttcctgctttatttaaggtcatcttctttgagcaaaaactgatgacgaaacaaacgttgttgcaacagtttttaacactttcgacacaagctcgaaaagaagggttgttgtcactcgaaacagcactcgaagaagtggataatgccttcatgcgccgtggtgtcatgatggtcatcgacggacaaccgtccgaatatgtcgaagatgtcatgacccgcgatctcgaaaacatgacagaacgccatcacgccaacgccaacatctttacgcaagctggtacatatgcgccgactcttggtgtactcggagccgtcatcggactcgtcgctgccctgtccgacctgtcggacatcgaaaaattgggccatgcgatttccggtgcgttcatcgcaaccctgttcgggattttcacgggatatgtcctctggttcccgttcgctaccaaactcaagcaaaaatcagccaatgaaatccaactgtatgaaatgatgatcgaagggattttatcgattcagaatggagagtcccctaaaaatctggaggataagctacttgtgtatctgacaccgaaggagcgtgcgacgtatgaaacggaaaaagaagccgcatga

SEQ ID NO: 85 - DNA, 783 bp, Bacillus cereus
  source 1..783: mol_type = genomic DNA
atgcaatcactttatacatcagccagcacgatggctcagctccagaagcagctcgatacgacgggacataatctggcgaatgccaatacgaacggctataaacgtcgggactctcagtttaatgaactgttggtccgcaatctcaacaatcagccgggcggtcttgtgaccggaccgttgacgacaccggaagggctgcggctcggcgtcggtggatatgtcgccaatgaagcgacacggtttacgacagggacgttccagaatacgggacggaaactcgatgctgcgctcagcaatccacatcatttctttggtgtgattgatgcggacggtgtgacgaaattcacgcgggacggcaattttgaattgtcaccgcaagcgaacggacaagttctgttgacggatgatgccggacgttctgtcatcaatcaggcgaatgagccgattacgtttccggatacggcgacatcgattgaactgaacaaagacggcaacatcacgggcatcttgaacggggaacgacgggtgctcgcccggattggcgtcgccgatattccgaaccacggcgaattgacggatgttggtgccggactcttcacggcgacgggacaataccagaatgcggcaggaaatccgttgacggtcggcacactcgagacgtcaaacgtcgacatggggacggaaatgacgaacttgacgcagattcagcgggcctaccagttcaattccaaagccttgacgacatcggatcagatgatgggaatcgtgacgtcgcttaagtaa

SEQ ID NO: 86 - DNA, 984 bp, Bacillus cereus
  source 1..984: mol_type = genomic DNA
atgaacagacttcgtcaaaaaccatggctcggtctagtcctcatgtcactccttgtcaccgtgctcagtttcctgttgcttggcatcggttccgtgtttctgaagccgggtgaaatcgtcgcggctcttcaaggagacggcgccggttccttcatcgtctggaactaccggttgccgcggacgctcctcgcgttactcgccggcggttgttttgccttgtccggtgtcttgttacaggcgattatccgcaatccgctcgtctcaccggatgtcatcggggtgacgaatggggccgcgttgttcgccgtcttgacgattgctttgattcctgatggtccgcttgttttgacaccgattgccgccttaataggagcaacgcttgtgatggtcgcgttgatgctgctggcggatcacgggaaactgcaaaacagttcctttgccttgctcggcatcgccgtcagtgcaatctgtgcatcgggaacggaatacctgttgatcaagtttcctctccagaccaatgattcgctcgtctggctcgccggcagcatgttcggcaaaggttggacggaagtgtacgtcctggcaccggtcttcctgttgcttggactcgtcatctggtccggtcaccggcaactcgacatcttatcactcagtgaagacgcagcgattggtctcggattacggatgaagggaacacggtatgtgttcctcgcctttgccgtcgctttggcaggtgttgcggtcgcaatggtcgggtcaatcggttttcttggcctcgtcgccccgcatatggcacggcgcttgatcggacaccggcatcatctgttgattccgatggctgtcctcgttgggggtggactgcttgtcgttgcggacgcgctcggacgcggcatccatccgccgcttgagattccggccggattaatcacggcaatcatcggtgtgccgtacttcctgtatctgttgcgaaaagaacgggcgtga

SEQ ID NO: 87 - DNA, 993 bp, Bacillus cereus
  source 1..993: mol_type = genomic DNA
atgatccgacacatacgattgatccgcttgctcgtcgtactccttgtcctgatcggactcggatcctacctcagtctgtttctcggcgtcacgacgattcaaccgcttgaagccattcgggaatggtcatccggcaacttgtcgaaagagacactggtcttgacgacactccgcttgccgcggttgttactcggtttattactcggggcaaacttagccgtcgccggtgccttgatgcaggcggtcacacgtaatccgttggcttcgccgcaagtgttcggcgtcaacgccggggcgtcgctgtttgtcgtcctcgctttgttgttgtttccggcacttgggacagcgaatctggtctatttcgcctttttcggtgcaatggttggcggattactggtcttttcgttcgcctctgtccgcggcatgacgagtctgaaactggccctcgtcgggatggcgatccacttgttgctgacgtccttgacgaaagggttgattttgttcaacgaccggatcaccaacgtcctgtactggttatccggttcaatcagtgacagtggatggatcgaagtccggttgattttaccctggtcgatcatcggcttgatcttagccttcagcctggccaaatcgctggcgattttccaactcggtcaagatgtggccgtcggactggggcagaacatcacccggattcggatgctggcagccgtcgctgttgtcctgctggccggggtgacggtcgcggtcgccggagcaatcggcttcatcggtctgatggtcccgcatatcgtccggcgattggtcggtgaggattaccgctatgtcttaccgatttcggcgttgtgcggtggtctgttgctgacatatgctgatgtcctcgcccggttcatcgcctatccgtatgaatcaccggtcgggatcgtgaccgcgttactcggagcgccgttctttttgtatttagcgaaacggcagacaagggggattgcctaa

SEQ ID NO: 88 - DNA, 927 bp, Bacillus cereus
  source 1..927: mol_type = genomic DNA
atgtcacgtacacgcacatcatgggcattagccgtcttgatggtcagttgtctgatgcttgcggcatgcgccggacaagcaaaagaagagacgaaacagacgcataaagtcacgcacgaagcagggacaacaaacgttccggacaatccgaaacgcgtcgtcgccctggaattctcattcgtcgacgcgcttgacgaactggggatcgaaccggtcggcatcgcccaagaaaacaaagacgatgtgtcgggtctgctcggcaagaagatttcctttacggaagtcggaacacgccagcaaccgaatctcgaagtcatcagttcgctgaaaccggacttgatcatcggtgacttcaaccggcataaaggaatctacaaacagttgcagcaaatcgcaccgacgatcattttaaagagccggaacgcgacgtatcaggaaaacatcgcgtcgtttaagagcatcgcggaagcggtcggtcagacggataagatggatcaacgtctcgagttacacgaagagcgtctcgcaacagccaaacaaaaagtcgatccgaacgatcaacgtcagattatggtcggtgtcttccggtcagattcgttgacggcacatggcgaaacatcgtttgacggcgaattgctcgaaaagatggggattgataatgccatcacgaaaacagcggaaccaaccgtgacgatcacactggaacagatcgtcaaatgggatccggatgtcatcttcatggcagaagccgatccgaagttgcttgatgagtggaaaaagaatccgctgtggaatcaaatcacagccgtcaaaaaaggggaagtctacgaagtcaatcgtgacttatggacccgttaccgtggactcgacgctgcggaacaaatcgtcgatgaagccattcaactgctgaatcaaacaaacaagtaa

SEQ ID NO: 89 - DNA, 840 bp, Bacillus cereus
  source 1..840: mol_type = genomic DNA
atggaacaaaaattaaagttatggttcacggaacaccaaacggaagattacggtatcacattccgtgtcaaccacgtttatgagagcgaacaaacggagtttcaacgcctggagatggttgagacggacgagttcggtacgatgttgttacttgacggaatggtcatgacaacagatcgagatgagtttgtatatcacgaaatggtcgcacacgttccactgttcacacacccgaatccaaaatcggttctggttgttggtggaggagacggcggcgtcatccgcgaagtgttaaaacacccatcagtcgaaaaggctgttcttgtcgaaatcgacggaaaagtcatcgaatactcgaagaaatatctaccaaacatcgcaggtggtctcgacgacgcacgtgttgaagtcatcgtgggagacggcttcatgcatatcgcagaagcagtcaatgaatatgatgtcatcatggttgactcgacagaacctgttggtcctgccgttaacctgtttacaaaaggtttctactcaggaatctcaaaagcattaaaagaagacggcatcttcgtcgcacagtcggataacccatggttcacaccagacttaatccgtgacgtccaacgcgatgtcaaagaaatcttcccaatcacgaaactctacattgccaacgttccgacttacccgagcggtctgtggacattcacgatcggatcgaaaaaacatgatcctctcgctgtcgcaccagagcgtttccacgagatcgaaacgaagtactatacaccggaacttcacacagcagcattcgcgctaccgaagttcgtcaaagatttaacgatttaa

SEQ ID NO: 90 - DNA, 873 bp, Bacillus cereus
  source 1..873: mol_type = genomic DNA
atgcgttttgatgaagcttattcaggtaaggtatttatcgcgagtcaaccgactcacgaagatgcaaaaggtgtcttgtacggcatgccgatggactggacggtcagtttccgtcccgggtcacgatttggtccggcccggatccgtgaagtgtcactcggactcgaagaatacagtccgtatctcgacggtgacattgcggatgcgaaattgtttgatgccggcgatatcccgttgccgttcggcaatgcccaaaagtcactcgacatgatcgaagaatacgtcgattcgttgctgacggcaggaaagtttccgcttggtatggggggcgaacacctcgtcacatggccggtcgtcaaagcctttgacaaacattatgatgactttgttgtgctgcactttgatgcacatacggacttacgcgattcgtatgaaggagaaccgttgtcgcactcgacaccacttaaaaaaatcgcaaacttgatcggaccggaaaactgttattcattcggcattcgttcagggatgaaagaagagtttgaatgggcgaagacgtccggttacaacttgtttaaatacgaaatcgtcgaaccgttaaaagctattttaccgaagcttgccggtaaaaaggtttacgtgacgatcgatatcgatgtgctcgatccttcggcggcacccggaaccgggacgcaggaaatcggtggtgtgacgacaaaagaattacttgaagtcgttcatatgattgcacgtgcggatgtcgacgtcattggagccgatttggttgaagtctgtccggcgtatgatcagtctgacatgacagcgattgcggctgccaaagtcttacgtgaaatgatgattgggtttatcaagtaa

SEQ ID NO: 91 - DNA, 1695 bp, Bacillus cereus
  source 1..1695: mol_type = genomic DNA
atgacagaaaaacaaaacagtgaaaaagaaaatatggtacagaagaaaacaggtgccgatttagtcgtcgatacactgattgaacaaggggtcgactatatttttggtattccgggagcaaagattgactccgtcttcaacgtccttcaagatcgtggaccggaattgattgtcgcacgccacgaacaaaacgccgccttcatggcacaagccatcggccgcttgactgataaaccgggtgtggtcctcgtaacttccggaccaggggcttcgaacctcgcaaccggacttgtgacggccaattcggaaggtgaccccgttgtcgcgattgccggtgccgtgacacgtgccgatcgtttgaaacggacccatcaatcgatggacaatcaagcgctcttcacaccaatcacgaatttcagtgccgaagttcaagatgcagacaacatcccggaagtcctttcgaatgcattccggactgccgaaacaacatccggcgctgctttcgtcagcattccgcaagacgttggtctcagtgaatcaaacgtcacttcctttaaagcggtcccaacaccaaaactcggcatcgcacccgaagaatggatcaacgagacagcaaatctgatcgaaaaagcacaattgccggtcttgttactcgggatgcgttccagtcagccccatgtcgtcaaagccattcgtgcactgttgaaacgcgtttcgattccggtcgtccagacattccaggcagccggaacattgtcacgggagctcgagtcgaatttctacggacgtgtcggtttgttccgcaatcaaccgggagatgccttgctcgctgaagccgatctcgtgttagctgtcggatatgatccgatcggttacgatccgaagttctggaatcaaccttcacacgaacggactttgattcacttggatcaaatgcgggctgaaatcgaccatttctatcgtccggatcgggaacttgtcggggatgtcgctgcaacaatcgacgcattggctgatcgactcaatccgctgagcctgccaaccagctcgacggaatttttacgcggtttacaacaacgcctcgaggagcgagatattccaccgattgtcaaggattcaccgttaacacatccgctgtatttcatgaaaaccttacgggaacaaatcgctgacgatgtgacggttacggtcgacgtcggttcgcactacatctggatggcacgtcatttccggtcttacgaaccgcgtcacttactgttcagtaacgggatgcagacgttaggtgtcgcattaccttgggcgattgccgcaacactggttcgtccaggcaaaaaagccgtctcgatctcaggtgatggtggtttcctcttctcggcgatggagcttgagacagccgtccgtttgaatgccccgctcgtccatttcgtttggcgcgacagcggctttgatatggtggctttccaacaagagatgaaatacaaacgaaaatccggcacgtcgttcggtgaagtggatcttgtgaaatatgctgaaagctttggtgcaaaaggcttgcgtgtcaatcatccgtctgaactcgtcgccgtcatggaagaagcatggcaaacagaaggtccggtcatcgttgatgttccaatcgattacagcgataacattacactcggtaaagaagtacacttggatcaactcaactga

SEQ ID NO: 92 - DNA, 714 bp, Bacillus cereus
  source 1..714: mol_type = genomic DNA
atgcagcgtgaggacaccttacttcaaatctcgacgatgatgtctttgctcgatggtgtttttgagagcgaaacaagttatgcctccattctcgaaggacatgactttggaatcggaacgtttgatcacctcgatggtgaaatgattggttttgacggttccttctaccaactccggtcagacggcagcgcacgtcctcttgatccggagacgaccactcccttttgttcactgacacggttcacgccggaacagacgctgtccgttgatcaggaaatgacaaaacaggattttgaacaatggttgagtgaacaactcggtacaatcaacagtttttatgctgtccggatcgaaggacagtttagtgaagtcaagacacggacggtcgcccgccaagaaaaaccgttccgtccgattacggaagccgtggccacgcaaagtgcccggacgttcgagcagacggaaggcacactggccggctattacacgccccggtttggtcacggtatcgcagtcgccggttatcatctccactttatcgacaaggaacgaagtggcggtggccacgtgtttgactatacggtcaatcgcgtgacggtcacgttcgaagagaaaccgcggctcgatcttcgactgccgacgacaactgcttaccgcgaggcggatcttgaaagtcacgatatcgaacaagaaatcaaaatcgcagaaggttaa

SEQ ID NO: 93 - DNA, 369 bp, Bacillus cereus
  source 1..369: mol_type = genomic DNA
atggcgttactgcttgcgcatcatcgtcctgtcgcccggaccgtttcctgggccggggtgacaaaccttgtctggacgtatgaagagcagcagacgatgcggaagatgttgcgacgcttcacgggtgggttgccggagcaacaaaaagaagcttatcaagttcgttctccgctctattttccgccgcaaggcgatgtcttgttgattcacggcttgtacgatcaaaatgtccgattgcgtcatgcgacgaattacgccgcccgttatccaaagcagacgcatttaaaagtgtatcaatatgcccatcaattcccgattcgtcaaaaattcgaagtgacggatgatgtcatcaactggatgatgacgtga

SEQ ID NO: 94 - DNA, 1419 bp, Bacillus cereus
  source 1..1419: mol_type = genomic DNA
atgaaaggtcggatgccgattgttgaagcactgtatgctcatgtagaaaggaaagctacttcttggcacgttcccggtcataaaaacggaacagtactaaacggcttaccttcttttttagaatgggataaaacagagttgactggtttagatgattttcatcatcctgaagaagccatctacgaagcaaaacttttgttgcgtcaaatctatgatgcctcggatagtcattttctggtgaatggatcaactgtcgggaattgggcgatgttagcggcggttgcgagtcgtggagaccggatctatgtgcaacggaactcgcataagtctgtttttaatgcgttggaatggttggggttatcgcccgttttaatggaaccggactaccatgcaacaggaatcagcggaaatgtttcacgtgaaacattagaagaggccttgaagctgtatcctggaggagtggcagtctttttgacgtcacccacctattatggagaaagcgccgagattgataaatgggttcatttgacgaagtcgcacgggttacccttactcgtcgatgaagcacatggtgcacattttggggaagcttttggagttcgttctgcctttgagttaggtgcaaccgctgttgttcaatctgcccataagactttacctgcattgacgatgggagcttggatccatgaacgtttcacggatgacgaacgaagacgactaacacgagcgcttcaagcctttcaaacgtccagtccgtcctacttgctgatggcttcgcttgattttgcacgtgattaccgtcaacagtttacggttgaacagatgttggaaatacggaacagccatgaacactttcaccaacgactcaatcaacacactgagttggacgtatttacgtttgatgattggtcgcggatgattgtgtcctgccgcgggtattccgggaatcaagtgttggcagcattggctaagcagggtatagatgcagagtttgctctcggggaacatgtcgtttgtattttaccgttgcgcctcttaccggaatccgaatgtcatgcatggatcgaacaaatcaaacaggcactcgatatgatgaaaacagaaggaattcccgataaaaggtatatggaacaacctattatgggtaaaataaaggtatcatcacttgcttgtccactcgatcaactggaacgtaccgtggcagtcgaacggtcttttgaagaggcagtcgattctgtttcacttgagacaatcattccatatcctcctggagttccacttttactacggggtgaacgggtgacgcaagcacatatcgaaatgattaagcagtatacgaccacatccgtccatctccaaggtggggagcttctatatgaagggaaattgcgtgtgatacaggaaggaattacagaatga

SEQ ID NO: 95 - DNA, 1338 bp, Bacillus cereus
  source 1..1338: mol_type = genomic DNA
atgaagaaaacatggatcacgacaagcgtactgggaatgacattagtggcaggtgtcacggcgtatacatatgaagcaccacgacacgtcgaggcaaaaccacagacggagtcgaagaaaaaggtcaaaaatgtcatcatgatgattccggacggttattctgcgtcatatgcaacaaattatcgttggtacaacggcggtgacgagacagaactggatcgtcagctcaagggcatgatgcggacatattccgcgagttctaaagtaacggactctgctgcggcaggaacggcgatggcaacgggcacaaaaacgaacaacggcacgatcgggatgaatccaagtggacaggaagttgaatcaatctttgaccgggcggaccgtgtcggcaaatcaacgggactggtagcgacatctgccatcacacatgcgacgccggctgtcttcgcttcccacgtcgcttcacgtgccaacgaagcagacatcgcaaaacaatacatggatgagatgaaagtagatgtcttgcttggcggcggtcaaaagtatttctttgataaggaaaatggtggcgtacaagaagcaggaaacctcgtcaaaaaagcggaacgcgccggatatcaatatgccgactcgctggaaagtcttcaggagaccgatgggcgaaaagtgctcggcttgtttgccgaggaaggaatggcaccggaactcgatcgagaattgacgcaacaaccaagtttgtcgacgatgacaaaaaaagcaattcaaacgctgaataaagataaagaaggatttttcctgatggtggaaggcagtcagattgattgggcgggtcatgcacatgatgctgcctgggcgatgaaggattcggacgccttccacaaagccgtaaaagaagcgatgcgttttgcggcaaaagataaaaacacattggtcgtcgtagcaggcgatcatgaaacaggcgggatgacggttggcggttatgatgagtatgtggcaaagccggaagtcttgaagaacgtcaaagcaaccggtgaccagatggtccgtcaatttaatgatgatttgacgaatatcgcggaaatcgtcaaacaagaaacatcattttatttgactgctcaagaagtcaagacgctccaaactgctgattctaaaaaacgtgtcatgctgttgaatgaaatgatcagtaagcgggcctatgtcggttggacgacaactgttcatacaggtgtagatgttccgttgtatgcatacggtccccacagcgatcagtttgccggtttacatgacaatacggacttacccggtttaattgccaatgcgatgaaactcaagaaataa

SEQ ID NO: 96 - DNA, 1626 bp, Bacillus cereus
  source 1..1626: mol_type = genomic DNA
atgaacgccgctgaacggttcgttgactgtctcgaagcagaaggtgtgacacacattttcggtgtgccgggggaagagaacattacgttactcgaagcgatcagtaaatcagacatcaccttcgtgacgacacgtcatgaaacaaatgcggcattcatggcctcgatgttcggccgattgagcggacgccccggcgtctgcctttcgacgctcggacccggggcaacgaatatgatgaccggtatcgccagtgcgacgatggatcattctccggtcgtcgcgattactgggcaaggggcgacgtggcggcagcataaagcttcgcatcagatgttcgatctggttgagatgtaccagccgatcacaaaatccagcacatcgatctcttccggtgaagtcatttcagaagtcgtccgccaggcgtttgctcaagctgcatccgaaaaaccgggcgcgacccacatctccttcccggaagacatcgctaaagctgacgtcgatgccaaaactcctttgcttgtggatagtgctccaacgtatctgcatccgacctcggtcaaggacagtgaagtcttgcgtcaaatggaacaagcggaaaaacctgtcgtgattgcgggattcgggattaatcggagcggagcgacggatgcctttcgtcacttcgtcgaacgtttaggtgcccccgtcgtcgagacgatgatgggaaaaggaacgattgcgtccgatcatgaactcgctgctcacacgatcggtctgccaaacgctgattataatcaacgcatcatcgaccaaagcgatttaatcattgcgattggttatgacattacggaactgccgccgtcgaaatggaacccgaaccggacaccggtcctccacatcgacacgaatcaacatgaggtggaccaatattatccggtcgtggccaatttgattggttctttgccggatacgttacggttacttgccgaggacgtgccgaatcgtgcctggtccggttggcaacaagaccgcgatcgattacgaaaggaaattcaggcaccctactcgatggcactgccgcttcatcctcaaagcatcgtccgggaactggaaaaggcgaccggttcggacgggatggtcttttccgatgtcggcgcgcacaaagtttggctcggacggcattttcaaacgacacgcccgaatcaattgttcatttcgaacggcttttcctcgatggggtacgggttatcaagtgccatcgccgcgaaactgctttatccggaccggcgtgtcctctgtgcgtcaggtgacggggcctttttaatgaatggtcaggatcttgagacggctgttcggctgaaattgccgatcgtcgtcatcatctggcgcgacgggacgtatggactgatcgaatggaaacaacagcaagcttacggacgggccccttatatcgaattcgacaatccggatctcgttcaactcgctgtcgcctttggcgcacttggtctgcgtgtcggagaacacggcacactggctgcttgtctcgagcaagctttcttgagtgacggacccgttttaattgactgtccggtcgattacagggagaatctgaagttaagtgaccgtttacgaacttatggaggatga

SEQ ID NO: 97 - DNA, 1086 bp, Bacillus cereus
  source 1..1086: mol_type = genomic DNA
atggatcaacatgcagaattaaaacgcttacgtgatgaactcgacctggtaaacgcagaattacttggattaatcaataaacggggcgagatagcggttgaaatcggtaaagtaaaacgggcgcaaggaatcgatcggtatgatccggttcgtgaacgccagatgcttgaatcgattgcagcaagtaatcacggtccatttgaaacaggacgcctacagcacgtatttaaagaaattttcaaagcctctttggaactgcaaggagaagaccggacacgcaagttgcttgtgtcgcgtaaacaaaagccgacgaataccatcatccggatcggagatgacgtcatcggtgatggctcgcagcagttaattgccggtccttgtgccgtcgaaagtgaggagcaggtctttgaagtggccgagcaactcgcgaagcatggggtacgcttcatgcgtggtggagcttacaaaccacggacatcaccatacgatttccagggtctcggtttagaaggattgaagatgctaaaaaaggcagctgatgcccatggtcttcatgtcattacagaaatcatgacgccgagtgcagttgagtcggctttaccatacgtggacatcattcaagtcggtgcccgcaatatgcaaaacttcgatcttttgaaagaagtcggccgaacggacaaaccggttctgttgaaacgtggtttgtcagcgacgctcgaagaattcatgtatgcagcagagtacatcatggcaagcgggaatgagcaggtcatcttgtgtgaacgtggtattcggacgtacgagcgtgcgacacgaaacacattggatatctcagctgttccgattttgaagcaagaaacacatttgcctgtcatggtcgatgtaacgcattcaacgggtcgtaaagacctgctcctgccgacagcgaaagcggcatacgcaatcggtgcagatgccgtcatggtagaagttcatccgttccctgcgcttgcgctctcggatgcgaaccaacaattagattttaatgagtttgattcgttcatcgagaatctgacttccacttttccggcactaaacgttcaatga

SEQ ID NO: 98 - DNA, 318 bp, Bacillus cereus
  source 1..318: mol_type = genomic DNA
atgaatttgctagacaatgcgatccgttatacggaaacgggcacgattcaggtgcagctcaggcaagagcctagccatgtggttacgatcgtggaagacagcgggatcggcattcccccagaggaattgccttctatttttgagcgcttttatcgggtggaaaaatcgcgttcccgagaacatggaggtaccgggttaggacttgctatcgtcaagcagctggtggacatgcagggcggaacgattcaagtatccagcgtgttaggaaaaggcactcgtttcgaaattacacttccgattggaggggatagccagtga

SEQ ID NO: 99 - DNA, 270 bp, Bacillus cereus
  source 1..270: mol_type = genomic DNA
gtggatgttctttttgaatcgctgcttccgctcaaggagttgaagcgtcatatcgtgatcatgaccggcatgggatcggacggcgccaaagggatgctggctctgaaagaatcgggagctgtaaccacgattgccgaatcggaagaaacctgtatcgtctacggtatgccccgagcagcggtcgagcttaaaggtgtcatgcatgtgctgaagcagcaagaaattgcaggcaaattaatatccgcaatcggtttatcggccttatcttag

SEQ ID NO: 100 - protein, 237 aa, Klebsiella pneumoniae
MRKLTLAFAAASLLFTLNSAVVARASTPQPLWVGTNVAQLAEQAPIHWVSVAQIENSLLGRPPMAVGFDIDDTVLFSSPGFWRGQKTFSPGSEDYLKNPQFWEKMNNGWDEFSMPKEVARQLIAMHVKRGDSIWFVTGRSQTKTETVSKTLQDDFLIPAANMNPVIFAGDKPGQNTKTQWLQAKQIKVFYGDSDNDITAAREAGARGIRVLRAANSSYKPLPMAGALGEEVIVNSEY

SEQ ID NO: 101 - protein, 431 aa, Escherichia coli
MKAILIPFLSLLIPLTPQSAFAQSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAISRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIKTALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSL

SEQ ID NO: 102 - protein, 594 aa, Pseudomonas fluorescens
MATVMKKVDAVIVGFGWTGAIMAKELTEAGLNVLALERGPMQDTYPDGNYPQVIDELTYSVRKKLFQDISKETVTIRHSVNDVALPNRQLGAFLPGNGVGGAGLHWSGVHFRVDPIELRMRSHYEERYGKNFIPKDMTIQDFGVSYEELEPFFDYAEKVFGTSGQAWTVKGQLVGDGKGGNPYAPDRSDHFPLESQKNTYSAQLFQKAANEVGYKPYNLPSANTSGPYTNPYGAQMGPCNFCGFCSGYVCYMYSKASPNVNILPALKPLPNFELRPNSHVLRVNLDSSKTRATGVTYVDGQGREIEQPADLVILGAFQFHNVRLMLLSGIGKPYDPITGEGVVGKNFAYQNMATIKAYFDKDVHTNNFIGAGGNGVAVDDFNADNFDHGPHGFVGGSPMWVNQAGSRPIAGTSNPPGTPAWGSAWKKATADYYTHQVSMDAHGAHQSYRGNYLDLDPVYRDAYGLPLLRMTFDWQENDIKMNRFMVEKMGKIAEAMNPKAIALLGKKVGEHFNTASYQTTHLNGGAIMGTDPKTSALNRYLQSWDVHNVFVPGASAFPQGLGYNPTGLVAALTYWSARAIREQYLKNPGPLVQA

SEQ ID NO: 103 - protein, 796 aa, Escherichia coli
MAINNTGSRRLLVTLTALFAALCGLYLLIGGGWLVAIGGSWYYPIAGLVMLGVAWMLWRSKRAALWLYAALLLGTMIWGVWEVGFDFWALTPRSDILVFFGIWLILPFVWRRLVIPASGAVAALVVALLISGGILTWAGFNDPQEINGTLSADATPAEAISPVADQDWPAYGRNQEGQRFSPLKQINADNVHNLKEAWVFRTGDVKQPNDPGEITNEVTPIKVGDTLYLCTAHQRLFALDAASGKEKWHYDPELKTNESFQHVTCRGVSYHEAKAETASPEVMADCPRRIILPVNDGRLIAINAENGKLCETFANKGVLNLQSNMPDTKPGLYEPTSPPIITDKTIVMAGSVTDNFSTRETSGVIRGFDVNTGELLWAFDPGAKDPNAIPSDEHTFTFNSPNSWAPAAYDAKLDLVYLPMGVTTPDIWGGNRTPEQERYASSILALNATTGKLAWSYQTVHHDLWDMDLPAQPTLADITVNGQKVPVIYAPAKTGNIFVLDRRNGELVVPAPEKPVPQGAAKGDYVTPTQPFSELSFRPTKDLSGADMWGATMFDQLVCRVMFHQMRYEGIFTPPSEQGTLVFPGNLGMFEWGGISVDPNREVAIANPMALPFVSKLIPRGPGNPMEQPKDAKGTGTESGIQPQYGVPYGVTLNPFLSPFGLPCKQPAWGYISALDLKTNEVVWKKRIGTPQDSMPFPMPVPVPFNMGMPMLGGPISTAGNVLFIAATADNYLRAYNMSNGEKLWQGRLPAGGQATPMTYEVNGKQYVVISAGGHGSFGTKMGDYIVAYALPDDVK

SEQ ID NO: 104 - protein, 261 aa, Bacillus megaterium
MYTDLKDKVVVITGGSTGLGRAMAVRFGQEEAKVVINYYNNEEEALDAKKEVEEAGGQAIIVQGDVTKEEDVVNLVQTAIKEFGTLDVMINNAGVENPVPSHELSLDNWNKVIDTNLTGAFLGSREAIKYFVENDIKGNVINMSSVHEMIPWPLFVHYAASKGGMKLMTETLALEYAPKGIRVNNIGPGAMNTPINAEKFADPEQRADVESMIPMGYIGKPEEVAAVAAFLASSQASYVTGITLFADGGMTKYPSFQAGRG

SEQ ID NO: 105 - protein, 372 aa, Bacillus amyloliquefaciens
MTATRGLEGVVATTSSVSSIIDDTLTYVGYDIDDLTENASFEEIIYLLWHLRLPNKTELAELKKQLAKEAAVPQEIIEHFKSYPLNNVHPMAALRTAISLLGLTDSEADVMNPEANYRKAIRLQAKVPGIVAAFSRIRKGLDPVEPKEEYGIAENFLYTLNGEEPSPIEVEAFNKALILHADHELNASTFTARVCVATLSDIYSGITAAIGALKGPLHGGANEAVMKMLTEIGEVENAEPYIRSKMEKKEKVMGFGHRVYKHGDPRAKHLKEMSKRLTNLTGESKWYDMSIRVEEIVTSEKKLPPNVDFYSASVYHSLGIDHDLFTPIFAVSRMSGWIAHILEQYDNNRLIRPRAEYTGPDKQTFVPIDERA

SEQ ID NO: 106 - protein, 312 aa, Bacillus cereus
MTIKRKKVSVIGAGFTGATTAFLLAQKELADVVLVDIPQLENPTKGKALDMLEASPVQGFDANIIGTSDYADTADSDVVVITAGIARKPGMSRDDLVATNSKIMKSITRDIAKHSPNAIIVVLTNPVDAMTYSVFKEAGFPKERVIGQSGVLDTARFRTFIAQELNLSVKDITGFVLGGHGDDMVPLVRYSYAGGIPLETLIPKERLEAIVERTRKGGGEIVGLLGNGSAYYAPAASLVEMTEAILKDQRRVLPAIAYLEGEYGYSDLYLGVPVILGGNGIEKIIELELLADEKEALDRSVESVRNVMKVLV

SEQ ID NO: 107 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVAKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 108 - protein, 257 aa, Bacillus cereus
MIEFRNVSKVYPNGTKGLNNINLKIQKGEFVIMVGLSGAGKSTLLKSVNRLHEITEGEIMIECESITAAKGKDLRRMRRDIGMIFQSFNLVKRSTVLKNVLAGRVGYHSTLRTTLGLFPKEDVELAFQALKRVNILEKAYARADELSGGQQQRVSIARALAQEAKIILADEPVASLDPLTTKQVLDDLKKINEDFGITTIVNLHSIALARQYATRIIGLHAGEIVFDGLVEAATDEKFAEIYGDVAQKSELLEVAAK

SEQ ID NO: 109 - protein, 280 aa, Klebsiella pneumoniae
MNSSLAAVAETDFQPFTDPAAGRQRKVLSVRNLSKAYQAQHKVLDGISFDLHAGEMVGVIGRSGAGKSTLLHVLNGTHSASGGEILSYPEVGTPHDVSQLKGRALNAWRSHCGMIFQDFCLVPRLDVLTNVLLGRLSQTSTLKSLFKIFPAADRARAIALLEWMNMLPHALQRAENLSGGQMQRVAICRALMQNPGILLADEPVASLDPKNTQRIMDVLREISEQGISVMVNLHSVELVRAYCTRVIGVASGQLIFDDHPSRLTQDVLQRLYGDEVSQLH

SEQ ID NO: 110 - protein, 303 aa, Bacillus mesophilus
MKKLWVMMIIALFAVLAACGGGNDEVKEEEQVENTEEQTASEELEKLVVGVIPSLNQGNMQTAMDKLSKHFESELDIPVEITVYPDYQAVVQAMNYDEVNMAYFGPSTYIDANEQSGARAIMTQLIDGEPFYYSYIITHKDSPLTSIEDLVAQSKDLTFAFGDPSSTSGSLIPSIELKKQGVFTNQNEHQFDNLLYTGGHDATALAVENKQVDAGAIDSAIFDTLQANGKIGDNFKIIWQSEKLFQYPWAVSKVVSDELVAKIQDAFLNVKDQEILDAFAATGFTVATDADYEAIREAKKEAK

SEQ ID NO: 111 - protein, 312 aa, Klebsiella pneumoniae
MKKYMTGAVRLSAMVAGMMMAWQAAAAQPKELNLGILGGQNATQQIGDNQCVKAFLDKELNVDTKLRNSSDYSGVIQGLLGGKVDVVLSMSPSSYASVYLNNPKAVDIVGIAVDDKDQSRGYHSVVIVKADSPYKTLDDLKGKAFGFADPDSTSGYLIPNHAFKEKFGGNADNKYNNTFSSVTFSGGHEQDILGVLNGQFAGAVTWASMVGDYNTGYTTGAFNRLIRMDHPDLMKQIRIIWQSPLIPNGPILVSNALPADFKAKVVAAVKKLDTEDHACFIKAMGGTQHIGPGSVADFQQIIDMKRELVSAR

SEQ ID NO: 112 - protein, 290 aa, Klebsiella pneumoniae
MKTTHTEFERYYQQVRSRQKRDAVCWSLLLLALYFAAGSAAEFNLLTIWHSLPHFFDYMAETIPPLSAGNLFADVQTKGSLAWWGYRLPIQLPLIWETLQLALASTLVAVAIATVFAFLAANNAWSPAPVRFAIRVLVAFLRTMPELAWAVIFVMAFGIGAIPGFLALMLHTVGSLTKLFYEAVESAQNKPVRGLAACGASPLQKIRFALWPQVKPLFLSYGFMRLEINFRSSTILGLVGAGGIGQELMTNIKLDRYDQVSITLLLIILVVSALDMLSGRLRLWVLEGKK

SEQ ID NO: 113 - protein, 264 aa, Bacillus cereus
MNDVTIYSKSIPKPPSKLKHMLTAVLVILLLWGSSVQVDASLSKLVVGFPNMMDLLKEMVPPDWSYFQVITTAMLDTIRMAIIGTTLGAILAIPLALFAASNVFTSTFLYSPARMILNFIRTIPDLLLAAIFVAIFGIGPLPGILALTFFSIGLVAKLLYESIESIDPGPLEAMTAVGANKVKWIVYGVIPQVKAHFVSYVLYTFEVNVRAAAVLGLVGAGGIGLYYDRTLGFLQYQQTASIIIYTLVVVLLIDYVSTLLREKL

SEQ ID NO: 114 - protein, 146 aa, Pseudomonas sp.
MNLSPRQHWIGVLARAQLNELQPHEAALKDAEYQLIRAPEIGMTLVRGRMGGNGAPFNVGEMTVTRCVVRLADGRTGYSYLAGRDKVHAELAALADAHLQNTPPSPWLTDLISALAQAQARRRAQKEADTAATKVEFFTLVRGENG

SEQ ID NO: 115 - protein, 136 aa, Klebsiella aerogenes
ADDTTNAVSIRQFFKRVTGVATTERTEDATLIQTRHRIPETPLTDDQILIFQVPIPEPLRFIEPRETETRTMHALEEYGVMQVKLYEDIARFGHIATTYAYPVKVNGRYVMDPSPIPKFDNPKMHMMPALQLFGAG

SEQ ID NO: 116 - protein, 471 aa, Escherichia coli
MKQSTIALALLPLLFTPVTKARTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 117 - protein, 461 aa, Bacillus subtilis
MKKMSLFQNMKSKLLPIAAVSVLTAGIFAGAELQQTEKASAKKQDKAEIRNVIVMIGDGMGTPYIRAYRSMKNNGDTPNNPKLTEFDRNLTGMMMTHPDDPDYNITDSAAAGTALATGVKTYNNAIGVDKNGKKVKSVLEEAKQQGKSTGLVATSEINHATPAAYGAHNESRKNMDQIANSYMDDKIKGKHKIDVLLGGGKSYFNRKDRNLTKEFKQAGYSYVTTKQALKKNKDQQVLGLFADGGLAKALDRDSKTPSLKDMTVSAIDRLNQNKKGFFLMVEGSQIDWAAHDNDTVGAMSEVKDFEQAYKAAIEFAKKDKHTLVIATADHTTGGFTIGANGEKNWHAEPILSAKKTPEFMAKKISEGKPVKDVLARYANLKVTSEEIKSVEAAAQADKSKGASKAIIKIFNTRSNSGWTSTDHTGEEVPVYAYGPGKEKFRGLINNTDQANIIFKILKTGK

SEQ ID NO: 118 - protein, 229 aa, Escherichia coli
MARRILVVEDEAPIREMVCFVLEQNGFQPVEAEDYDSAVNQLNEPWPDLILLDWMLPGGSGIQFIKHLKRESMTRDIPVVMLTARGEEEDRVRGLETGADDYITKPFSPKELVARIKAVMRRISPMAVEEVIEMQGLSLDPTSHRVMAGEEPLEMGPTEFKLLHFFMTHPERVYSREQLLNHVWGTNVYVEDRTVDVHIRRLRKALEPGGHDRMVQTVRGTGYRFSTRF

SEQ ID NO: 119 - protein, 576 aa, Zymomonas mobilis
MNSLLHHSFLKTVFSSLAIAIVTSSLSSVTIAATHPLDNHPKGEIAASSETAHNPWSGTRLIVAISVDQFSSDLFSEYRGRFRSGMKQLQNGVVYPMAYHSHAATETCPGHSVLLTGDHPARTGIIANNWYDFSVKRADKKVYCSEDPSLSADPQNYQPSVHYLKVPTLGDRMKKANPHSRVISVAGKDRAAIMMGGHMTDQIWFWSDNAYKTLADHKGEMPVTVKTVNEQVTRFMQQDEAPVMPSVCADHASALKIGNNRIIGLAPASRKAGDFKTFRVTPDYDRTTTDIAIGLIDELKLGHGNAPDLLTVSLSATDAVGHAYGTEGAEMCSQMAGLDDNIARIIAALDSNGVPYVLVLTADHGGQDVPERAKLRGVETAQRVDPALSPDQLSLRLAERFQLSHNQPLFFANEPQGDWYINRNLPEQTKAQLIQAAKSELSNHPQVAAVFTASELTHIPYPTRSPELWNLAERAKASFDPLRSGDLIVLLKPRVTPIAKPVSYVATHGSAWDYDRRVPIIFYTPHASGFEQPMPVETVDIMPSLAALLQIPLRKGEVDGRCLDLDPTEATTCPVK

SEQ ID NO: 120 - protein, 583 aa, Bacillus subtilis
MAYDSRFDEWVQKLKEESFQNNTFDRRKFIQGAGKIAGLSLGLTIAQSVGAFEVNAAPNFSSYPFTLGVASGDPLSDSVVLWTRLAPDPLNGGGMPKQAVPVKWEVAKDEHFRKIVRKGTEMAKPSLAHSVHVEADGLEPNKVYYYRFKTGHELSPVGKTKTLPAPGANVPQMTFAFASCQQYEHGYYTAYKHMAKEKLDLVFHLGDYIYEYGPNEYVSKTGNVRTHNSAEIITLQDYRNRHAQYRSDANLKAAHAAFPWVVTWDDHEVENNYANKIPEKGQSVEAFVLRRAAAYQAYYEHMPLRISSLPNGPDMQLYRHFTYGNLASFNVLDTRQYRDDQANNDGNKPPSDESRNPNRTLLGKEQEQWLFNNLGSSTAHWNVLAQQIFFAKWNFGTSASPIYSMDSWDGYPAQRERVINFIKSKNLNNVVVLTGDVHASWASNLHVDFEKTSSKIFGAEFVGTSITSGGNGADKRADTDQILKENPHIQFFNDYRGYVRCTVTPHQWKADYRVMPFVTEPGAAISTRASFVYQKDQTGLRKVSSTTIQGGVKQSDEVEEDRFFSHNKAHEKQMIKKRAKITN

SEQ ID NO: 121 - protein, 250 aa, Salmonella typhimurium
MKSRYLVFFLPLIVAKYTSAETVQPFHSPEESVNSQFYLPPPPGNDDPAYRYDKEAYFKGYAIKGSPRWKQAAEDADVSVENIARIFSPVVGAKINPKDTPETWNMLKNLLTMGGYYATASAKKYYMRTRPFVLFNHSTCRPEDENTLRKNGSYPSGHTAYGTLLALVLSEARPERAQELARRGWEFGQSRVICGAHWQSDVDAGRYVGAVEFARLQTIPAFQKSLAKVREELNDKNNLLSKEDHPKLNY

SEQ ID NO: 122 - protein, 431 aa, Escherichia coli
MLERLSWKRLVLELLLCCLPAFILGAFFGYLPWFLLASVTGLLIWHFWNLLRLSWWLWVDRSMTPPPGRGSWEPLLYGLHQMQLRNKKRRRELGNLIKRFRSGAESLPDAVVLTTEEGGIFWCNGLAQQILGLRWPEDNGQNILNLLRYPEFTQYLKTRDFSRPLNLVLNTGRHLEIRVMPYTHKQLLMVARDVTQMHQLEGARRNFFANVSHELRTPLTVLQGYLEMMNEQPLEGAVREKALHTMREQTQRMEGLVKQLLTLSKIEAAPTHLLNEKVDVPMMLRVVEREAQTLSQKKQTFTFEIDNGLKVSGNEDQLRSAISNLVYNAVNHTPEGTHITVRWQRVPHGAEFSVEDNGPGIAPEHIPRLTERFYRVDKARSRQTGGSGLGLAIVKHAVNHHESRLNIESTVGKGTRFSFVIPERLIAKNSD

SEQ ID NO: 123 - protein, 540 aa, Bacillus subtilis
MNKYRVRLFSVFVVCMILVFCVLGLFLQQLFETSDQRKAEEHIEKEAKYLASLLDAGNLNNQANEKIIKDAGGALDVSASVIDTDGKVLYGSNGRSADSQKVQALVSGHEGILSTTDNKLYYGLSLRSEGEKTGYVLLSASEKSDGLKGELWGMLTASLCTAFIVIVYFYSSMTSRYKRSIESATNVATELSKGNYDARTYGGYIRRSDKLGHAMNSLAIDLMEMTRTQEMQRDRLLTVIENIGSGLIMIDGRGFINLVNRSYAKQFHINPNHMLRRLYHDAFEHEEVIQLVEDIFMTETKKCKLLRLPIKIERRYFEVDGVPIMGPDDEWKGIVLVFHDMTETKKLEQMRKDFVANVSHELKTPITSIKGFTETLLDGAMEDKEALSEFLSIILKESERLQSLVQDLLDLSKIEQQNFTLSIETFEPAKMLGEIETLLKHKADEKGISLHLNVPKDPQYVSGDPYRLKQVFLNLVNNALTYTPEGGSVAINVKPREKDIQIEVADSGIGIQKEEIPRIFERFYRVDKDRSRNSGGTGLG

SEQ ID NO: 124 - protein, 240 aa, Bacillus subtilis
MNKKILVVDDEESIVTLLQYNLERSGYDVITASDGEEALKKAETEKPDLIVLDVMLPKLDGIEVCKQLRQQKLMFPILMLTAKDEEFDKVLGLELGADDYMTKPFSPREVNARVKAILRRSEIAAPSSEMKNDEMEGQIVIGDLKILPDHYEAYFKESQLELTPKEFELLLYLGRHKGRVLTRDLLLSAVWNYDFAGDTRIVDVHISHLRDKIENNTKKPIYIKTIRGLGYKLEEPKMNE

SEQ ID NO: 125 - protein, 633 aa, Pseudomonas sp.
MRFNCKPCLLPLLISLSAGHAQAATPVTAPTLKPWSATKAQALGWLAGDQRLAVSKREGVLLLDAQGKTLSHVPGAFASLDSRALGDQVLVASHDEKKQQVALFSLNPQSHEWLAPVYLPRRDYAVNGVCLYRDEASNIYLFTVGEEGKGEQWLVAADRKRLNQPRLVRSLPLPPEAGLCQVDDAAHQLFVNEQKVGWWAYPAHAEAQASRVPVAMIEPFGEVKQAAGAMVPVPGGMLGLDPKAGELHLYQQQGKGWSPVARFPLKPLVEPEHLAVRQTPQGLDVWVQDADNNQLFEGRLSWNPVPVSVPPVLPVVKPSVQTDPVVSQGDAADDPAIWLHPHDLALSRVLGTNKKNGLEVYDLQGRRVQHLEVGRLNNVDVRPDFKLGTRTVDLAVATNRDHNSLSVFSIDRATGEVRAAGEVPTPLKDIYGLCLFKAPTGEIYSFANDKDGTFLQHRLSAKGEQVQGELVRQFKVATQPEGCVADDTHQRLFIGEEDVAVWALDARPEQPAALSSVINVGGPVHDDIEGLALYQGEKNSYLVISSQGNDSYVVLDAQPPYALRGAFRVGVNAEAGIDGASETDGLEVTSANLGGPFTQGMLVVQDGRKRMPEHSQNYKYIPWADVAKTLNLP

SEQ ID NO: 126 - protein, 383 aa, Bacillus subtilis
MNHSKTLLLTAAAGLMLTCGAVSSQAKHKLSDPYHFTVNAAAETEPVDTAGDAADDPAIWLDPKTPQNSKLITTNKKSGLVVYSLDGKMLHSYNTGKLNNVDIRYDFPLNGKKVDIAAASNRSEGKNTIEIYAIDGKNGTLQSMTDPDHPIATAINEVYGFTLYHSQKTGKYYAMVTGKEGEFEQYELKADKNGYISGKKVRAFKMNSQTEGMAADDEYGRLYIAEEDEAIWKFSAEPDGGSNGTVIDRADGRHLTRDIEGLTIYYAADGKGYLMASSQGNSSYAIYDRQGKNKYVADFRITDGPETDGTSDTDGIDVLGFGLGPEYPFGIFVAQDGENIDHGQKANQNFKIVPWERIADQIGFRPLANEQVDPRKLTDRSGK

SEQ ID NO: 127 - protein, 263 aa, Klebsiella pneumoniae
MHLVKTILTAGLLLSAAAQAHNVLEFPQPENNPEEFYAVTEIPTGGIIKYETDAKTGFIVADRFQSMPVAYPANYGSLTQSLAGDGDPLDVVFYTRAPMAPGTLIKLRAIGVLKMIDGGEKDDKIIAVPASKIDPTYDDIKTISDLPKIEQQRLEAFFRVYKELPEGRKRSSWPALMTPRPRSRRSNRPGRPGRRRTRNNIHRGRCPAPAAFAFTIPMAAKSHVTRHTTTLIPPYCILTQDEGDGCIVQPFHFLDPPPCLIPP

SEQ ID NO: 128 - protein, 165 aa, Bacillus sp.
MAFENKIVEAFIEIPTGSQNKYEFDKERGIFKLDRVLYSPMFYPAEYGYLQNTLALDGDPLDILVITTNPPFPGCVIDTRVIGYLNMVDSGEEDAKLIGVPVEDPRFDEVRSIEDLPQHKLKEIAHFFERYKDLQGKRTEIGTWEGPEAAAKLIDECIARYNEQK

SEQ ID NO: 129 - protein, 939 aa, Bacillus sp.
MSSELPAITPQNTQDPAGDTDLRRDVRRVSTLLGESLVRQHGPELLAMVEQVRLLTKESKEAARGETGTGPWSANDVAEQVREVLASLPLEQATDLVRAFAFYFHLANAAEQVHRVRSLRARAEEDGWLARTVAEIAEKAGAGALQKVVNELDVRPIFTAHPTEASRRSVLDKVRKLSDILATPSAEGSSARSRQDRQLAEIIDQMWQTDELRKVRPTPMDEARNAIYYLNNILTDAMPEVLTELSGLLGAHGVTLPEDAAPLRFGSWIGGDRDGNPNVTADVTRDVLVLQNANAVKISVAMVDELLSVLSNSSSLSGADAQLLESITVDLANLPGIAPRVLELNAEEPYRLKLTCIKAKLLNTRRRVAADAHHEPGRDYASTAELLADLALLETSLRNNSAALVADGALAAVRRAVASFGLHLATLDIREHADHHHDAVGQLLDRVGELPGPYAELDRAGRLQVLSRELASHRPLSGHPIKLDGAADGTYDVFRVVRRALRTYGPDVVETYIISMTRGADDVLAPAVLAREAGLVQLTGDNRYAKIGFAPLLETVEELRASGEIVDRLLSDPSYRELVRLRGNVQEIMLGYSDSNKESGVMTSQWEIHKTQRKLRDVAVKHGVNVRMFHGRGGSVGRGGGPTYDAILAQPNGVLEGAIKFTEQGEVISDKYSLPELARENLELSLAAVMQGSALHQAPRHTEDDLVRFGEVMEIVSGAAFASYRALIDDGDLPAYFLASTPVEQLGSLNIGSRPSKRPDSGSGLGGLRAIPWVFGWTQSRQIVPGWFGVGSGLKAAREAGREAELAEMLAHWHFFSSTISNVEMTLAKTDMDIAAHYVRTLVPESLAHLFATIRAEYDLTVAEIQRLTGEAELLDKQPLLKRSLNVRDQYLDPISYLQVELLRRVREAGIAKAEVDERLQRAMLITINGVAAGLRNTG

SEQ ID NO: 130 - protein, 510 aa, Klebsiella pneumoniae
MPINDNTPRPQEFAAVDLGSNSFHMVIARVVDGAMQIIGRLKQRVHLADGLDENSVLSEEAMTRGLNCLSLFAERLQGFSPSSVCIVGTHTLRQATNAAEFLKRAEKVIPYPIEIISGNEEARLIFMGVEHTQPERGRKLVIDIGGGSTELVIGEDFEPRLVESRRMGCVSFSQAYFPGGVINKENFQRARLAAVQKLETLAWQFRIQGWTVALGASGTIKAAQEVLVAMGEKDGFITPERLEMLVNELLKHKNFDALSLPGLSEDRKAVFAPGLAILCGVFDALAIKELRLSDGALREGVLYEMEGRFRHQDIRSRTAQSLANQYNIDREQARRVLETTTQMLEQWQEQNPKLANPHLAALLKWAVMLHEVGLNINHSGMHRHSAYILQNSDLPGFNQEQQMLMATLVRYHRKAIKLDDLPRFTLFRKKQFLPLIQLLRLGVLLNNQRQATTTPPTLRLQTEAHHWTLTFPHNWFSQNALVLLDLEKEQQYWEGVPEWMLKIAEEEPDA

SEQ ID NO: 131 - protein, 512 aa, Bacillus cereus
MKEILKQQYAIIDIGSNTMRLVIYEKQNGGFYKEIENTKVVARLRNYLVDGVLIEEGIEVLLQTLFQFQESTRFHQLHHVLCVATATIRQAKNQEEIKKLVEGQTDFTLRVLSEYEEARYGYLAVMNSTSFSEGITVDIGGGSTEVTYFRNREILEYHSFPFGALSLKQQFIKDDIPTEEELEKLQTYLEYQFRTLPWLIDKKLPLIAIGGSARNLVKIHQNLICYPIAGVHLYKMKEEDIKNVKEELEALSFIELQKLEGLAKDRADTIVPAVEVFHTLVNVIEAPAFVLSRKGLREGVFYEELTKDLGISYYPNVVEESLYLLSHEYEMDMEFVMQLIKHGRLICQQLEETGLISMSVEDWEVFHQAAKVFNIGKYIDEEASRLHTFYLLANKTIDGMMHKERVRLALIASYKSKMLFKQHLSPFEGWFDKNEQKKIRLLGAVLQFSAALNIRQRSLVESISITESKEGLTFEIVCEQSALAEKVQAEKQKKQLERVLKTNIILLFKLKN

SEQ ID NO: 132 - protein, 23 aa, Klebsiella pneumoniae
MWKKPAFIDLRLGLEVTLYISNR

SEQ ID NO: 133 - protein, 237 aa, Aneurinibacillus migulanus
MAITSYEQIEEKIWDIVETEIIQGEFMQTLLAGEWTPAQVREFALQYSYYSRNFPRVLGAAIAAVEPEDDWWVPLVDNLWDEGGRGNPKSYHSRLYHSFMITAAPDVPTNEKYVPDYPVSPASKEAVNTFISFLRNATPLEAMASIGFGSELFAGKVMGLIGQGLEHPNYNRAQKLNTTFWTVHADHHEPRHYELCKNVLTRFTSSQDLEHMYRAGAYITRSEARFYDGLYERMKSV

SEQ ID NO: 134 - protein, 23 aa, Klebsiella pneumoniae
MWKKPAFIDLRLGLEVTLYISNR

SEQ ID NO: 135 - protein, 342 aa, Klebsiella aerogenes
MNVMRTTVATVVAATLSMSAFSAFAAASLTGAGATFPAPVYAKWADTYQKETGNKVNYQGIGSSGGVKQIIANTVDFGASDAPLADDKLTQEGLFQFPTVIGGVVLAVNLPGVKSGELVLDGKTLGDIYLGKIKKWDDEAIAKLNPGLKLPSQNIAVVRRADGSGTSFVFTSYLSKVNEEWKSKIGAGSTVNWPTGLGGKGNDGIAAFVQRLPGSIGYVEYAYAKQNNLAYTKLVSADGKPVSPTEDNFANAAKGVDWSKSFAQDLTNQKGENAWPITSTTFILVHKATNKPEQTAEVLKFFDWAYKNGGKEANALDYATLPEKRGRAGSRGMENQRQRQQR

SEQ ID NO: 136 - protein, 300 aa, Bacillus subtilis
MKKNKLVLMLLMAAFMMIAAACGNAGESKKSNSDSAKGEEKASGSLTISGSSAMQPLVLAAAEKFMEENPDADIQVQAGGSGTGLSQVSEGAVQIGNSDVFAEEKEGIDAKALVDHQVAVVGMAAAVNPDAGVKDISKDELKKIFTGKIKNWKELGGKDQKITLVNRPDSSGTRATFVKYALDGAEPAEGITEDSSNTVKKIIADTPGAIGYLAFSYLTDDKVTALSIDGVKPEAKNVATGEYPIWAYQHSYTKGEATGLAKEFLDYLKSEDIQKSIVTDQGYIPVTDMKVTRDANGKQS

SEQ ID NO: 137 - protein, 438 aa, Klebsiella pneumoniae
MISLRHTALGLALSLAFAGQALAVTTIPFWHSMEGELGKEVDSLAQRFNAANPDYKIVPVYKGNYEQSLSAGIAAFRTGNAPAILQVYEVGTATMMASKAIKPVYQVFSEAGIKFDESQFVPTVAGYYTDSKTGHLLSQPFNSSTPVLYYNKDAFKKAGLDPDQPPKTWQDLAAYTAKLKAAGMKCGYASGWQGWIQIENFSAWHGLPVATKNNGFDGTDAVLEFNKPEQVKHIALLEEMNKKGDFSYFGRKDESTEKFYNGDCAITTASSGSLADIRQYAKFNYGVGMMPYDADVKGAPQNAIIGGASLWVMQGKDKETYTGVAKFLDFLTKPENAAEWHQKTGYLPITTAAYDLTRQQGFYDKNPGADIATRQMLNKPPLPFTKGLRLGNMPQIRTIVDEELESVWTGKKTPQQALDSAVQRGNQLLRRFEQATKS

SEQ ID NO: 138 - protein, 458 aa, Bacillus thuringiensis
MSLVKKGAALLMAATMALSSAACSNSKTEGKPEALAKVAPVEKNGDKTVIRFWHAMGGKTQGVLDGLVADYNKSQNKYEIKAEFQGTYEESLTKFRTMSATKEAPALVQSSEITTKYMIDSKKITPIDSWIKKDKYDTSKLEKAITNYYSVDGKMYSMPFNSSTPVLIYNKDAFAKAGLDPEKAPKTYAELQEAAKKLTIKEGGNVKQYGFSMLNYGWFFEELLATQGALYVDNENGRKDAAKKAVFNDKEGQKVFGMLDDLNKAGALGKYGASWDDIRAAFQSGQVAMYLDSSAGVRDLIDASKFNVGVSYIPYPEDSKQNGVVIGGASLWMTNMVSEETQQGAWDFMKYLTKPDVQAKWHTATGYFSINPDAYNEPLVKEQYEKYPQLKVTVDQLQATKQSPATQGALISVFPESRDAVVKALEAMYDGKNSKEALDEAAKATDRAISISARTSQK

SEQ ID NO: 139 - DNA, 1527 bp, Klebsiella sp.
  source 1..1527: mol_type = genomic DNA
atgccaataaacgaaaaaacccctcggccgcaggagttcgctgcggtcgaccttggttcgaacagttttcatatggtgatcgcccgtgtcgtcgacggggcaatgcagatcatcggtcgcctgaagcagcgcgtccacctggccgatggcctggatgaaaactcggtgttgagcgaagaagcgattactcgtgggctgaactgcctgtcgctgtttgcggaacgtctgcagggattctccccttccagcgtttgtatcgtcgggacgcataccctgcgccaggcggctaacgcggcggattttctcaaacgagcggaaaaagttatcccttatcccatcgaaataatctccggtaacgaagaagcgcgtctgatttttatgggcgtcgaacatacgcaaccggagcgcggccgtaagctggttatcgacatcggcggcggttcgactgagctcgtcatcggcgaagatttcgagccccgcctggttgaaagccgacgtatgggctgcgtaagcttctcgcaggcctatttcgccggcggcgttatcaataaagaaaacttccagcgcgcgcgcctggcggcggtgcagaaactggaaaccctggcctggcagttccgtattcaggggtggaacgtggcgctgggcgcctctggcaccatcaaagccgcgcacgaagtgctggtcgccatgggggagaaagacggcttcattaccccggaacgtcttgaaatgctggtcagtgagcttctgaagaataaaaacttcgactcattaagcctgccgggattgtcggaggaccgcaaagcggtattcgccccgggtctggcgattttgtgcggggtattcgacgcgctggcgatcaaagaactgcgcctgtccgacggcgccctgcgcgaaggcgtgttgtacgagatggaaggtcgcttccgccaccaggatattcgcagccgtacggcccagagcctggcgaaccagtacaatatcgatcgcgaacaggcgcgccgggtactggagactaccactcagatgctggaacagtggcaggagcaaaacccgaaactggctaacccgcatctgtctgccctgctgaagtgggcggtaatgctacatgaagtggggctgaacattaaccacagcggcatgcatcgccactctgcctatatcctgcaaaatagcgatctgccgggctttaatcaggagcagcagacgctgatggccacgctggtgcgctatcaccgcaaagctatcaagcttgatgatttgccgcgttttaccttgttcaagaaaaaacagttcttgccgctgatccaactactgcgtctgggcgtattactgaacaatcagcgtcaggcgaccactacgccgccaaaactgacattgaagacagaagccaaccactggacgttaattttcccgcatgactggtttagccagaatgcgttggtgctgctggatctggaaaaagagcaacagtactgggaaggcgtgccggaatggctgctgaaaattactgaagaagaagcgtaa

SEQ ID NO: 140 - DNA, 1662 bp, Klebsiella sp.
  source 1..1662: mol_type = genomic DNA
atgcaaccgacttacaccattggcgattatctgctggatcgtctcgtagattgtggtatcgatcgcctgttcggcgtacctggtgattacaacttacagtttctcgatcacgtgatagcccatcaagatttgggatgggtcggctgtgctaacgaactgaacgcggcctacgccgcagacggctatgcgcgtataaaaggcgctggcgcgctgctaaccacctacggcgtaggggagttaagcgcgttgaatggggtggccggtagttatgcggaacatatcccggtgctgcatattgtgggcgccccttccactggcgcgcagcagcgcggtgaactcctgcaccatacgctcggcgatggcgatttcacccatttctcgcggatgagcgaacaaattacctgtactcaggcgacgctaacggcgggcaacgcctgtcatgaaattgaccgggtattaagcgacatgctgacccaccaccgcccagggtatctgatgctgcctgccgacgtggcgaaagcccgtgcggtgccgccggcccgcgcgctggtcattaagggcccggcggccgatgaaaaccagcttgccggctttcgcgagcacgcggccaaattgttgcgcagcagtcgccgcgtatcgctgctggcggattttctcgctcagcgttacggtctgcaaaacacactacagcagtgggttaagtccgcgcctattacccacgccaccatgctgatggggaaagggctatttgacgaacaaggctcaggttttgccgggacctatagcgggatcgccagcgcgccgcagactcgcgaggcgatggaaagcgccgatgccatcatctgcgtgggtacgcgctttaccgatacgattaccgccggctttacccatcatttgccaacggagaaaaccatcgaaatccagccgttcgccgcgcgggttggcgatcactggttcagccggatcccgatggatcaggcgctggcggcactcattgacgtttctggcaagctgtccgccgaatggagcgcgccagatatcatcgcgcccgacgcttccggcgcgccgcaggggaatctgacgcagaaaagcttctggagtacggtcgagaaacagctgcggccgggcgacattattcttgccgaccaggggacctcggcattcggcatcgcctcattaaaacttccctcccaggcgacgctgctggtgcagccgctatggggctcaatcggctttacgctacctgccgcctacggcgcacagacagccgccgcggacagaagggtggtattaatcattggcgatggcgccgcccagctcactatccaggagatgagctcaatgctgcgcgataagcaaaagctgttaattttgctgttgaataatgagggctacaccgtcgagcgcgcgattcacgggccagaacagcgctacaacgatatcgcattgtgggactggagccgcttccccgatgcttttgcgccggatacgccttcccgctgttggcgggtaacgcaaaccggcgaattggcggaggcgatggccgatagcattaattccgataagttaacgatggtcgaagtcatgctgccgaaaatggatattcctgatttcttacgcgcggtcacccaggcgctggaaaatcgcaacaaccgcggctaa

SEQ ID NO: 141 - DNA, 1221 bp, Klebsiella sp.
  source 1..1221: mol_type = genomic DNA
atgaaacgtgcagtgattactggcttgggcatcgtttccagcatcggtaataaccagcaggaagtcctggcatctctgcgtgaaggacgttcagggatcactttctctcaggagctgaaggattcaggaatgcgtagccacgtgtggggcaacgtcaaactggataccacgggcctcattgaccgcaaagtagttcgctttatgagcgatgcatctatctatgcttatctgtccatggagcaggcggtagccgacgcaggtctggcgccggaagcataccagaacaacccgcgtgtcggcctgattgcaggttccggcggcggctccccgaaattccaggtcttcggcgccgatgccatgcgtagcccgcgtggtctgaaagccgtgggcccatacgttgtgactaaagcgatggcttccggcgtatctgcctgcctcgccacgccgtttaaaatccacggcgtcaactactctattagctccgcttgcgccacttccgcacactgcatcggtaacgcggtagaacagattcagctgggcaaacaggacatcgtctttgccggcggcggcgaagagctgtgctgggaaatggcttgtgaattcgacgccatgggcgcgctgtccactaaatacaacgattctccggacaaagcgtcccgtacctatgatgcgaaccgcgacggtttcgttatcgcgggcggcggcggcatggtcgtggtggaagagctggaacacgcgctggcgcgcggcgcgcatatctatgcggaaatcgtcggttacggcgcgacttccgatggcgcggacatggttgctccatctggcgaaggcgcagtgcgctgcatgcagatggcgatgcacggcgttgataccccgatcgactacctgaactcccacggcacttcgactccggtaggcgacgtgaaagagttgggcgctatccgcgaagtcttcggcgacaacagcccggctatctccgccaccaaagcgatgaccggccactctctgggcgccgcaggcgtacaggaagccatctactctctgctgatgctggagcacggttttatcgcgccaagcatcaacattgaagagatggacgagcaggctgccggccttaacatcgtcaccaagccgaccgatgctcagttgaccaccgtgatgtccaacagcttcggcttcggcggcaccaacgcgaccctggttatgcgtaaatacaacgcctaa

SEQ ID NO: 142 - DNA, 810 bp, Klebsiella sp.
  source 1..810: mol_type = genomic DNA
atggaacgttacgagacgctatttgcacagttaaaaaaccgccaggaaggcgcctttgttcccttcgtcaccctcggcgatccggggccggaacagtcgctgaaaatcatcgatgcgctgattgaagccggcgccgatgcgctggaactggggatccccttctccgacccgctggccgacggcccgacgattcagggcgccacattgcgcgcttttgccgccggcgttaccccggcgcagtgcttcgagatgctggcggcgatccgccacaagcatccgaccattccgatcggcctgttgatgtacgcgaacctggtgttcagcccggggattgatgagttctacgccgaatgcgcgcgtgtcggcgtggattccgtgctggtcgccgacgtgccggtcgaagagtccgccccgttccgtcaggcagcgctgcgccataacattgcgccgattttcatctgcccgccaaatgccgatgacgatttactgcgccagattgcctcctatggccgcggctatacctacctgctatcgcgcgctggcgtgacaggcgcggaaaaccgcgccgcgctgccgctgcatcatctgattgaaaaattggcggaatataacgccgcgccgccgctgcagggctttggtatctcggcgccggagcaggtttcggccgctattgacgccggtgccgccggggcaatatccggctcggccatcgtcaagatcatcgagcgcaaccttgaacagccgcaaaaaatgctcgaagagctaaaaaccttcgtacagagtctgaaagcggcgaccaaaaccgcctga

SEQ ID NO: 143 - DNA, 1194 bp, Klebsiella sp.
  source 1..1194: mol_type = genomic DNA
atgagcactttactgaacccgtatttcggcgaattcggcggtatgtacgttccgcagatcctgatgcccgccctgcgccagctggaagaggctttcgtcagcgcgcaaaaagatcctgagtttcaggccgaattcaccgacctgctgaaaaactacgcgggccgcccgacggcgctgaccaaatgccgcaatctgaccgaaggcacccgcaccacgctgtacctcaagcgtgaagatctgctccacggcggcgcgcacaaaaccaaccaggtgctgggccaggcgctactggccaaacgtatgggtaaaacggaaattatcgccgaaaccggcgccggccaacacggcgtcgcctcggcgctggccagcgccctgctcggtctgaaatgccgcatctatatgggcgccaaagacgtcgagcggcagtcgccgaacgtgttccgcatgcgcctgatgggtgcggaagtcattccagtgcacagcggttccgccaccctgaaagatgcctgtaacgaagcgctgcgcgactggtccggcagctacgagaaggcgcactacatgctcggcaccgccgctggcccgcacccattcccgactatcgtgcgtgaattccagcgcatgatcggcgaagaaaccaaagcgcagatccttgagaaagaaggacgcctgccggacgcggtgatcgcctgcgttggcggtggttctaacgccatcggcatgttcgctgatttcattgatgaatcccgcgttggcctgattggcgtcgagcctgccggccacggtatcgaaaccggcgagcacggcgcgccgctgaagcatggtcgcgtcggcatttatttcggcatgaagtcgccgatgatgcaaacctccgacgggcagattgaagaatcttattctatctccgccgggctggatttcccgtcagtggggcctcagcatgcgttccttaatagcaccgggcgcgctgagtatgtgtcaattaccgacaacgaagcgctggatgcctttaaagcgctctcccgccatgaaggcatcattccggcgctggagtcgtcgcacgctctggcgcacgcgctgaagatgatgcgcgagaatccggataaagagcagctgctggtggttaacctgtccggccgcggcgataaagacatcttcaccgtacacgacattttgaaagcgcgaggggaaatctga

SEQ ID NO: 144 - DNA, 1359 bp, Klebsiella sp.
  source 1..1359: mol_type = genomic DNA
atgcagaccgttttagcaaaaatcgttgccgacaaagcgatttgggtagaagcccgcaaacaacaacaaccgttagccagttttcagaatgacattgtgccgtccgaacgccatttttacgatgcgctggccggcgcccgcaccgcttttattctcgagtgcaaaaaggcgtcgccgtcgaagggactgattcgggaagatttcgacccggcgaccatcgccggggtttataagcactacgcctcggcaatttcggtactgtgcgatgagaaatattttcagggcagctttgattttctgccgatcgtcagcaaagtcgcgccgcagccgattctgtgtaaagacttcaccatcgacccttaccagatttatctcgcgcgttactaccaggccgatgcctgcctgctgatgctttcggtgctcgatgacgatcaatatcgccagctcgccgccgtcgcccacagtctcaatatgggcgtcctgaccgaggtcagcaatgaagaggagctggagcgcgcgattgcgctgaaggccaaagtcgtcggcattaacaaccgcgatctgcgcgatatgtcgattgatctgaaccgcacgcggcaattggcgccgcgtctcggcccggatgtcaccgtgatcagcgaatccggaatccatacctatggcgaagtccgcgagcttagccacttcgccaacggcttcctgattggctcggcgctgatggaacaaccggacctcagcgctgcggtgaagcgcgtgctgttaggtgagaacaaggtctgcggcctgactcgtccgcaggatgcgcaagtcgcctgggaggcgggcgccatctacggcggcctgattttcgtcgatagttcgccgcgtgccgttaacgatcagcaggcacaggcggtaatggccgccgcgccgcttagctacgttggcgtgttccgtgatgcagccgttgaagaggtcgtcacccgcgcgaccaaattgaaactcgccgcggtccagcttcatgggagcgaagatcaggcctggattgatgccctgcgcgcggcgctgccggagcagatccaaatctggaaggcgctgagcgtcggcgaaagtttaccgccgcgcaacctgaaccatgtgaccaggtatgtctttgacaacggtcagggcgggagcggtcaacgtttcgactggtcgctgctgcagggccaggacctgcgtaacgtgatgctggcaggcggcctcagcgccgataactgcgtagaagccgcgaaaagcggctgtgccggactcgatttcaactcaggcgtagagtcgcagccgggaataaaagaggcaagcctggtggctgccgttttccagacgctgcgcgcatattaa

SEQ ID NO: 145 - DNA, 1596 bp, Klebsiella sp.
  source 1..1596: mol_type = genomic DNA
atggccgacatcctgctgctcgataatatcgactcctttacctataacctcgccgaccagctgcgggctaacggccacaacgtggttatttatcgtaataccgtaccggcacaatcgctgattgaacgtatcggcaccatggataatccggtgctgatgctctctccggggccgggaaccccaagcgaagccggctgcatgcccgagctgctgacccgcctgcgcggcaagttaccgattatcggtatctgcctcggccaccaggccatcgtggaagcctacggcggctatgtcggccaggcgggagaaatccttcacggcaaagcgtcaagcattgagcatgacggccaggcgatgttcgccggtctcgccaacccgctgccggtggcccgctaccattctcttgtcggcagcaatattccggccgggctcaccattaacgccaatttcaacggtatggtgatggcggttcgccacgacgccgatcgcgtctgcggcttccagttccatccggaatcgattctgactacccaaggagcgctactgctggagcaaacgctggcatgggcgctgcagaaactggagcagaccaatgtcgtacagccgattctggaaaaactgtaccaggccgagacgctgagccagcaggagagccacgtgctgttttccgccgtcgtgcgcggcgaagtgaagccggaacagctggccgccgcgctggtaagcatgaaagtgcgcggcgaacaaccacaggaaattgccggcgccgctaccgcgctgctggaaaacgccgcgccgttcccgcgcccggattatcagtttgccgatatcgtcggtaccggcggcgatggcagcaacagtatcaatatctcaaccgccagcgcctttgtcgccgccgcctgcggtttaaaagtggcgaaacacggtaaccgcagcgtctccagtaaatcgggttcgtccgacctgctggccgcgtttggcatcaacctggatatgaatgccgataaatcgcgcgccgcgctggatgaactgggcgtctgctttctgttcgcgccgaaataccacaccggtttccgccacgcgatgccggtccgccagcagttgaaaacgcgcaccctgtttaacgtgttggggccgcttatcaacccggcgcacccgccgctggcgttgattggcgtctacagcccggaactggtgttgccgattgcagaaaccttgcgcgtactcggttatcaacgcgccgcggtggtacacagcggcggcatggacgaagtctcgttgcatgcgccgaccgtggtcgccgaactcaataacggcgaaatccagagctatcagctgaccgccgccgacttcggcctgacgccttatcatcaggagcaactggccggcggcacgccggaagaaaaccgtgacattctcacgcgcttgctacaaggtaaaggtgaagccgctcatgaagccgccgtggccgccaacgtcgccatgttgatgcgtttacacgggcatgaagacctgaaagccaacgcccggcaggtactggatgtgctgcacagcggagcggcctatgacagagtgaccgcattagcggcaagagggtaa

SEQ ID NO: 146 - DNA, 1563 bp, Klebsiella sp.
  source 1..1563: mol_type = genomic DNA
atgcaaacatcaaaaccagcgctcgagcttctcaccagcgacgccatctatcgcgaaaacccgacggcgttattccatcaattatgcggcgcgcgtccggcaaccttgctgctggaatcggccgacatcgatagtaaagacgatttaaaaagcctgctgctggtcgatagcgccctgcgcattaccgctcttggcaataccgtcactgtccaggcgctttcagcgaacggcgccgcgctgcttgaactgctggataacgcattgccgtccggcattgagaatctgcgccagccgaacagccgggtactcaccttcccaccggttagcgccctgctggatgaagacgctcgcctgtgttcgttatcggtgttcgatgcgtttcgtctgttacaggatttggtcagcgtcccgcaggccgagcgcgaagcgatgttcttcggcggcctgttcgcttacgacctggtggccggttttgaggatttaccaccgctcaataccgataccgcctgcccggattactgtttctacctggcggagacgctgctggtcatcgatcaccagaccaaaagcacccgcattcaggcgagcctgttcacgccgctggagaatgagaaacagcgtcttctgcaacgtatcgcccagcttcgccagcagttaaatgaaccgcccgcgccgctgccggtgacaacggttgccgaaatgcgctgcgacgtcgatcagagcgatgaagagtacggcgccgtggtgcgcaaaatgcagcgcgccattcgcgcgggcgaaattttccaggtcgtgccgtcgcgccgcttctccctcccctgcccgtcgccgctggcctcgtacgatgtgctgaaaaagagcaatcccagcccgtatatgttctttatgcaggataacgatttcaccctgttcggcgcatcgccggaaagctcgctgaaatacgacgccgtcagccgccagattgagatctaccccattgccggcacccgtccgcgcggccgccgcgccgatggttcgctggatcgcgatctcgatagccgtattgagctggagatgcgtaccgaccacaaagaactttctgaacatctgatgctggtcgacctggcccgtaacgatctggcccgcatctgtaccccgggtagccgctacgtggcggatttaaccaaagtcgatcgctactcctttgtgatgcatctggtttcccgcgtggtcggcgaactgcgccgcgatcttgatgtcctgcacgcctaccgcgcctgcatgaatatgggcacccttagcggcgcgccgaaagttcgcgccatgcagctaatcgccgccgcggaaggcaagcgtcgcggtagctacggcggcgcggtcggctactttaccgcccatggcgacctcgatacctgcatcgtgatccgctcagcctacgttgaggatggcattgctaccgtgcaggcgggcgccggtatcgtgctcgattccgttccgcaatccgaggcggatgaaacccgtaataaagctcgcgccgtgctgcgcgccattgctcaggcccaccacgcgaaggagactttctaa

SEQ ID NO: 147 - DNA, 2268 bp, Klebsiella sp.
  source 1..2268: mol_type = genomic DNA
atggcaaacggcttgatgttttcccctcgtaaaaccgcgctggcgctggctgtcgcggtggtttgcgcctggcaatcaccggtcttcgctcacggtagcgaagcgcacatggtgccgttggataaaacgctgcaggcgttcggcgccgatgtgcagtgggatgactacgcgcaaatgttcaccctgataaaagacggcgcttacgtcaaagtaaaacccggcgccaaaacggcgatcgtcaacggtaacccccttgatctacaggtgccggtagtaatgaaggaaggtaaagcctgggtctccgatacctttatcaacgatgtattccagtccggtctcgatcagaccttccaggtagaaaaacgccctcacccgttaaattcgctctcggcggcggaaatcggtgaagcagtgaccattgttaaagccgcgccggagttccagccgaatacccgctttactgagatttccttacacgagccggacaaggcggctgtatgggcctttgccctgcagggaacgcccgttgatgcaccccgcaccgccgatgtggtgatgctcgatggcaaacatgtcattgaagccgtcgtcgatctgcaaaacaaaaaaatcctctcatggacgccgattaaaggcgcccacgggatggtgctgcttgatgacttcgtcagcgtgcagaacattatcaatgccagcagcgagttcgctgaggtgctgaaaaagcacggtattaccgatcccagtaaggtggtcaccaccccgctcaccgtcggctactttgatggcaaagatggcctgcagcaggatgcacgtctgctgaaagtcgtcagttatctggataccggcgacggcaactactgggcgcacccgattgaaaacctggtggcggtggtcgaccttgaagcgaagaaaatcatcaaaatcgaagaaggcccggtgctcccggtaccgatggagccccgttcttacgatggtcgcgaccgcaacgccccggcggtgaaaccgctggagataaccgaaccggaaggcaaaaactacacgatcaccggcgataccattcactggcggaactgggattttcatctgcgcctgaactcgcgcgtcgggcccattctctcgacggtgacttacaacgataacggcacaaaacgccaggtaatgtatgaaggttccctcggcgggatgatcgtcccctacggcgaccctgacgtcggctggtatttcaaagcctatctggactccggcgattacggcatgggcaccctaacatcgcctattgttcgcggtaaagatgcgccgtcaaatgcagtactgctggacgaaactatcgccgattacaccggcaaaccgaccactattccaggcgcggtcgccatattcgaacgctatgccgggccagaatataagcacctggaaatgggcaagcccaacgtcagcaccgagcgcagggaactggtggtgcgctggatcagtaccgtcgggaactatgattatatctttgactgggtgttccacgacaatggcaccatcggtatcgatgccggcgccaccggcattgaagccgttaaaggcgtgaaggcgaagaccatgcacgaccccagcgccaaagaggatacccgctacgggacgctgatcgaccataatattgtcggcaccacccaccagcatatttataatttccgcctcgatctcgacgtggacggcgaaaacaacaccctggtggcgatggatcctgaagtgaagccaaacaccgccggcggcccgcgcaccagcaccatgcaggtgaatcagtacacaatcgatagcgagcagaaagcggcgcagaaattcgaccctggcactatccgcctgctgagcaacaccagcaaagagaaccgcatgggtaacccggtctcttaccagattatcccttatgccggcggcacgcatccggcggcgaccggcgcgaagttcgccccggacgagtggatatatcatcgtctgagctttatggataaacagctgtgggtgacgcgttaccacccgacagagcgttatccggaagggaaataccctaaccgttccgcccaggataccggcttaggccagtacgcgaaggatgatgagtcgctgacaaaccacgacgacgtcgtgtggatcaccaccggcaccacccacgtcgcgcgcgccgaagagtggccaattatgccgaccgagtgggcgcacgcgctgctcaagccgtggaacttctttgacgaaaccccaacgcttggcgagaagaaaaagtaa

SEQ ID NO: 148 - DNA, 2373 bp, Klebsiella sp.
  source 1..2373: mol_type = genomic DNA
atggctacaggcaacgtgccgcgcggattcccccggatcctgcagtggctactcgccggactgatgttaatcatcggtctggcaatcggcattttaggcgccaaactggcaagcgtcggcggcacctggtatttcgccattatgggactggtgatggttatcgcctccctgcttattttccgcaatcggcgcggcggcatcgttctttatgccgtcgccttcgcggcttccattgtttgggctatcagcgacgccggctggacctactggccgctgttctcacgcctgtttgcgcttggcgttctggcttttctgtgcgccattgtttggccttttctttccagcgcaccggcaaaaaaaggcgcggccttcgccctcgcgggcgtgctggccgtggcactgctggtcagttttggttggatgtttaaatcccagccgctggtcagcgccagcgaagcggtgccggtaaaaccggtacagccaggtgaagaacaaaagaactggcagcactggggtaataccactcacggcgaccgtttcgccgcgttggatcagatcaataaaaacaatatcaaccagctacaggttgcctggattgcccataccggcgatattccgcagagtaacggttccggtgcggaagaccagaatacgccgctgcagattggcgatacgctttatgtttgtacgccatatagcaaagtcctggcgctggatgtcgatagcggcaaagaaaaatggcgctacgactcgaaagcgacggcgcctaactggcaacgttgccgcggtttaggctactacgaagatagccaggcgcaggctaatgccgtggccgggactcagcctgccgcctgtgcccgccgcctgttcctgccgactaccgatgcgcgcctgatcgcaatcgatgccgataccggtaaagcctgtgaagcatttggtgaacacggtaccgtcgatctcagcgtcggcatgggggaaatcaaacctggttattatcagcagacctctaccccgctggtggccggtaaccttgttgtcgttggtggtcgcatcgcggataactactccaccggtgaaccgccgggtgtagttcgcgcttttgacgtgcataccggtaaactggcctgggcctgggatccgggtaacccgcagctgaccggcgtaccgccggaaggacaaacctacacacgcgggacgccgaacgtctggtccgcgatgtcttacgatgccaaactgaatctcatttatctgccaaccggtaacgccacgccagatttcttcggcggcgaacgtacggcactggatgataaatacagctcatcaatcgttgcggtcgatgccgccaccggtaaagtgcgctggcatttccagaccacccatcacgatctgtgggatttcgacctgccttcgcaaccactgctgtacgatctgccggacggtaaaggcggtacgacgccggtactggtgcaaaccagcaagcagggcatgatcttcatgcttaaccgcgcgacgggtgaaccggtggcgaaagtggaagaacgccctgtcccggccggcaacgtcaaaggtgaacgctactcgccgacgcagccctactccgtcggcatgccgatgatcggcaacgaaacgttgaaagaatcggatatgtggggcgccaccccggttgacctgctgctgtgccgtattcagttcaaagagatgcgccatcagggtgtctttacaccgccgggtgaagaccgctccttgcagtatccgggctcgcttggcggaatgaactggggcagcgtgtcggtcgatccgaataacggcctgatgttcgtcaatgacatgcgccttggactggctaactacatggtaccgcgagcgaaagtggctaaagatgccagcggtatcgaaatgggtatcgtaccgatggaaggtacgccgtatggcgcaatgcgcgagcgtttcctgtcgccgctgggcatcccctgccagaagccgccgttcggtaccatgtcggcggttgatctgaaaagcggtaagctggtctggcaggttccggtaggtacggtagaagataccggtccgctgggtatccgcatgcatatgccgatccctatcggcatgccaacgctgggagcttcgctgtctacccagtctggcctgctgttcttcgccggcacccaggatttctatctgcgcgcctttgataccgccaacggcaaagagatttggaaatctcgcctgccggtgggcagccaatccggcccgatgacctacgtttcaccgaaaaccggtaagcagtacatcattatcaacgccggcggcgcgcgccagtctccggatcgcggcgattacatcatcgcttacgctttagcggataagcagtaa

SEQ ID NO: 149 - DNA, 681 bp, Klebsiella sp.
  source 1..681: mol_type = genomic DNA
atgatacagtacttaaacgtcttcttttacgatatctacccttatctctgcggcaccgtgttcctggtgggcagttggctgcgctatgactacgggcagtatacctggcgcgcgtcatccagccagatgctcgataaacggggcatggtgctgtggtcgaacttattccatatcggcatcctcggcattttcttcggccacttcttcgggatgctgacgccgcactgggtctattcatggtttctgccgatgtcgcagaaacaggtgatggcgatggtgctgggcggtgtctgcggcgtactgaccctggtcggcggtattggcctgctggcgcgccgcctgaccaacccgcgcattcgcgccacctccaccactgcggatattctgatcctgtgcattctgctgattcagtgcgcgctggggctgacgaccatcccgttctccgcccagcatccggacggcagcgaaatgctgaaactggtggattgggcgcaggcggtagtcaccttccacggcggcgcatcggcgcatctggacggcgtcgcgtgggtgtatcgcgtccatctggtgctgggaatgactatcttccttatcttcccgttcacccggctggttcacgtctggagcgcgccgatagagtatttcacccgccgctaccaggtggtgcgctcccggcgctga

SEQ ID NO: 150 - DNA, 696 bp, Klebsiella sp.
  source 1..696: mol_type = genomic DNA
atgcggatcctcaaagtcattgctctgttgctggagtatcccgacgacgcgctgtgggataaccgcgaggaggcgctggcgctggtcagcgccgatgcgcctgccctgacgccgtttgttaggcgattactggcggcgccgctgctcgaccgccaggccgaatggtgtgaggtatttgaacgcggtcgcgccacctcgctgttgctgtttgaacacgttcacgccgaatcacgcgatcgcggccaggcgatggtcgatctgatgaaccagtatgaacaggcgggcctgcagatcgattgtcgcgaactgccggaccacctgcctttatatcttgaatacctcagcattctcccattagccgatgcccgtgaaggactgcagaacgtcgcgcctattctggcgttgctcggtggacgcttaaagcagcgcgaatgcgattactatcaactctttgacacccttcttgcgctggcggatagcccactcaatagtgacagtgtcaccaggcaggtagcgagtgagaagcgtgacgacacccgccaggcgctggatgcggtgtgggaagaggaacaggtgaagtttattgaagataatgcaaccgcttgcgatagctcgccgatgcaagcttatcaacgacgctttagccaggatgtggcgccgcagtacgtcgacatccgcgccggaggcccgaaatga

SEQ ID NO: 151 - DNA, 1545 bp, Klebsiella sp.
  source 1..1545: mol_type = genomic DNA
atgaaaatacgttcacaagtcggaatggtgctgaatctggataaatgcatcggatgccatacctgctccgtcacctgtaaaaacgtctggagcagccgcgaagggatggaatatgcgtggttcaacaacgtggaaaccaagccaggtattggttaccccaaaaactgggaagatcaggacgagtggcaaggcggctggatccgcggtatcagcggcaagcttactccgcgtctgggcaaccgcgtcagcgtgttgtcgaagattttcgccaacccggtgctgccgcagattgacgattattacgaaccctttacctacgattatcagcacctgcacaacgcgccggagggcaaatacttgcctaccgcccgcccgcgttcgctgattagcggcgaacggatggataagatcacctgggggccaaactgggaggaactgctcggcggcgagtttgaaaaacgcgccaaagaccgtaacttcgaagcgatgcaaaaagagatgtacggccagtttgaaaacaccttcatgatgtatctgccgcgtctttgcgaacattgcctcaatccgagctgcgtagcgacctgcccaagcggcgccatctataagcgtgaagaagacggtatcgtgctgatcgaccaggataaatgccgcggctggcggatgtgcatcagcggttgtccgtacaaaaaaatctactttaattggaagagcggcaaatcggaaaaatgcatcttctgctatccgcgtattgagtccgggcaaccgacagtctgttcagaaacctgcgttggccgcatccgctaccttggcgtgctgctgtacgacgcggaccgtatcgaagaagcggccagcactgaacatgaaaccgacctctacgagcgccagtgtgatgtgttcctcaaccccaacgatccggcggtcatcgaagaggcgttgaaacagggtattccacataacgtgatcgacgccgcgcagaaatcaccggtgtataaactggcgatggactggaagctggcgctgccgctgcacccggaataccgcaccttaccgatggtctggtacgtgccgccgctgtcgccgattcagtcggtcgccgacgctggcggcctgccgagtaacggtaacgtcctgccggcggtagaaagcctgcgtataccggtacagtatctggcgaacctgctgagcgccggtgataccggcccggtcttgcgcgcgttaaaacgcatgatggcgatgcgccattacaaacgttcgcaaaccgtcgaaggcgtgaccgatacccgtgcgattgaagaagtgggcctcagcgtcgaacaggtggaagagatgtatcgctacctggcgatcgccaattatgaagaccgcttcgtgatccccaccagccaccgcgaactggcggaggacgccttccctgaacgcaacggctgcggttttaccttcggcgatggttgccatggttctgatactaaattcaacctgttcaacagtcgtcgcatcgacgctattgacgtcggagatgttcgtcgacatggggagggagagtaa

SEQ ID NO: 152 - DNA, 3741 bp, Klebsiella sp.
  source 1..3741: mol_type = genomic DNA
atgagtaaattactggatcgctttcgctactttaagcagaaacgcgagacctttgccaacggccacggtcaggtctacgacaataaccgtgactgggaagatagctaccggcaacgctggcaattcgacaaaattgtccgctccacgcacggtgtgaactgtaccggctcctgtagctggaaaatttatgtcaaaaacggtctggtcacctgggaaacccagcagaccgattatccgcgcacccgaccggacctgccaaatcacgaaccgcgcggctgtccgcgcggcgccagctactcctggtatctctacagcgccaaccgcctgaagtacccgctggcgcgcaaacggctgattgagctgtggcgcgaagcacttacacagcatcccgacccggtacaggcctgggatagcatcatgcaggaccccctgaaaacccgcagctacaaacaaatccgcggtaaaggcggttttgttcgctccagttggaaagagttaaatcaactgatcgccgcagctaacgtctggaccatcaaaaactacggtccggaccgcgtcgcgggcttttcgccgatcccggcgatgtcgatggtttcctacgccgcgggtacccgctatttatcgttgatcggcggcacctgcctgagcttttatgactggtactgcgatttaccccccgcctcgccgatgacctggggcgaacagaccgacgtgccggaatccgccgactggtataactccagctacattattgcctggggttcaaacgtcccgcagacccgaaccccggacgcgcacttcttcaccgaagtgcgctacaagggcaccaaaaccatcgccattactccggattattcggaagtcgccaagctctgcgaccagtggctggcgccgaagcaaggtaccgacagcgcgctggcgatggcgatgggccacgtgatcctcaaaaccttccacctcgataacccaagcgattactttctcaactactgccgtacctataccgatatgccgatgctggtaatgctcgaccgtcgtgacgacggcagctatgcgcctggccgtatgctgcgcgccgccgacctgcttgacgggctgggagaaagtaataacccggaatggaaaaccgtcgcctataacgccgaaggcgagctggtcgcgccgaacggatccatcggctttcgttggggcgaaaaaggcaaatggaatctcgaacaacaggctaacggccgggacgtcgaattaaaattgtcgctgctggatatgcacgatagcgtggtgtcggtcggtttcccctacttcggcggcaacgaaaacccgcatttccgcagcgtgaagcaggagccggtaacgcttcatcagctgccagctaaacagctaccgttggccaatggtgagagcgctttggtggtgagcgtgtatgacctggtgctggctaactacggtctggatcgcggtcttggcgatgccaatgccgcgcgcgacttcgcggatgtcaaagcctataccccggcatgggccgagcagatcaccggcgtgccgcgtcaacatatcgaacaaatcgcccgcgagttcgccgatacggcgcataaaacccatggccgttcgatgattatcctcggcgccggtgtgaaccactggtaccacatggatatgaactaccgcgggatgatcaacatgctggtgttctgcggctgcgtcggtcagagcggcggtggctggtcgcactatgtcggccaggagaaactgcgcccgcagaccggctggctgccgctggcgttcgcgctggactggactcgtccgccgcggcagatgaacagtacctcttatttctacaaccacgccagccaatggcgctacgaaaaactgaccgctcaggagctgctgtcgccgctggccgacgccagcaaattcagcggccatatgattgacttcaacgttcgcgccgagcgcatgggctggctgccgtccgcgccgcagcttaacgttaatccgctgagcatcaggcagcaggccgaggccgccgggctgtcaccggccgatttcactgtccagtcgttaaaagcaggcgatattcgctttgccgccgagcagccggatagcggcaacaaccacccgcgcaacctgtttatctggcgttcgaacctgctgggttcgtccggtaagggccatgagtacatgctcaagtacctgctcggtaccgataacggtattcagggcgaggaatttggctccaccgctgacgtgaagccggaggaagtcgagtggcagaccgcggcgatcgagggcaaacttgatctactggtgaccctcgatttccgcatgtccagtacctgcctgttctccgatatcgtcctgccgaccgccacctggtatgaaaaagacgatatgaacacctcagacatgcacccgtttatccaccccctttccgccgccgtcgacccggcctgggaggcgaaaagcgactgggaaatctacaaggacatcgccaaaagtttctctgaagtgtgcgtcggccaccttgataaagagaccgatgtggtactggtaccgctgcagcatgactctccggcggagctgtcacagccgttcgaagtcctcgactggcgcaagggcgaatgcgatctcattccaggcaaaaccgcgccgtcaattgcgctggttgaacgtgactacccggccacctgggaacgcttcacctcgctggggccgttactcgataagctcggcaacggcggtaaaggcatttcgtggaatacgcaaagcgaggtcgacttcctcggcaaacttaactacgtcaagccagacggtccggccaaaggccgtccgcgtatcgacagcgctatcgacgccagtgaagtcatcttaagcctggcgccggaaaccaacggccaggtggcggtgaaagcctggcaagcgctgggagaatttaccggccgcgaccacactcatctggcgctgaataaagaggatgagaaaatccgcttccgcgatattcaggcgcagccgcgcaagatcatctccagcccaacgtggtctggtctggaaagcgaacatgtctcctataatgccggttataccaacgttcacgaactgatcccgtggcgtaccctctccgggcggcaacagctgtatcaggatcatccctggatgcgcgccttcggcgagagtctcgtggcctatcgcccaccgatcgatacgcgcagcgtcagccagatgaaggaagtgccgccaaacggcttcccagaaaaagcgctgaacttcctgaccccgcatcagaaatggggtatccactcgacctatagcgaaaacctgctgatgctgacgttgtcgcgcggcgggcccatcgtctggctaagcgaaaccgacgcaaaagaactggggattgaagataacgactggatcgaagccttcaacgccaacggcgccctcaccgcgcgtgcggtggtgagccagcgcgtgccgccgggcatgaccatgatgtatcacgctcaggaacgcattatgaacattccaggttcggaggtgaccggtcgccggggcggcatccacaactccgtgacccgcgtctgtcctaaacccacgcatatgattggcggctatgcccaactggcttacggctttaactactacggcaccgtcggctccaaccgcgacgagttcattatgatccgcaaaatgaaaaatattgactggctggatggcgaaggtcgtgaccaggtacaggaggcgaagaaatga

SEQ ID NO: 153 - DNA, 2298 bp, Klebsiella sp.
  source 1..2298: mol_type = genomic DNA
atgaccaccgccacccgcatcgtggaactggcaggcgggattcgcgcaacgctggttcatcagccgcaggcgacgcgtgctgcggcgttggttaaggtgggtgccggaagccatcacgaaaccgatgccctgccggggctggcccatttactggaacatttgctgtttcgcggcagccagcgctatcacgcagacgagcgattaatggcgtggatccaacgccagggcggcagcgtcaacgccaccacccttgcccgccacagcgcctatttcttcgaggtcgccgccagtagtctggccgagggcatcacccgtctacgggatacgctccaggcgccgctgctgtcgcctgaagatatccgtcaggaactggcggtcatcgatgcggaaaaccggcttatccagcagcacgatccctcccgccgtgaagccgcagcccgtcacgcgatggcggcgcccgcggcttttcgccgttttcaggtcggcagcatggattctctgggcggaaacttgcctgcgctgcaggttgcgctcggcgagtttcatcagcgctattatgtcgccagccatctgcagctatggcttcaggggccgcagtcgcttgatgaattagccgcattggcccatttttttgccgcgggattccctggcggcctgccgccggaaagcccgccgtctgcgtattttagcaacaatgtggatcttcaactggccgttgaagaccaaccggcggtatggcgttgtccgctcattgcagtaagtgacaacgtcactacttttcgtgaattcttactggatgaagcgcccggcagcctgctggccgggctacgtgaatgcgggctcgcggacgatgtcgcgctgaactggctttaccaggatgaacgcgtcggctggctggcgctggtatttaccagcgaccatcccgacgccatccagcagcatctcgagcgatggctgcaggcattgcgccacacctcgcctgaacagcagttgcactattacaggctggcgcagaggcgttttaacgcactcaccccgctcgaacagttacgtcagcgggcattgggttttgcgcccgattccccgcccatcgactttgcccgtttctgcgccagcttgctgacggcgccagcctcttcgctggtgtgccgcaaaatagcggctggcgatggcgtggcaacccagggctttaccttgccgctcggccgcaggcaaccacggacagtcgtgattgatccgctggcatttagcttctatccgcaggcggcgaccacggcagcgcctgacctgccgccaacgaccagcccgctgctgcatgtcattgcagataatcaaacgccaacgctgatcgttcgcaagccattctatagcgtgcttagtcaggcgcaggggatggcgataagccagcagctccgtcccttgctcgcgcaacttcgccatgctggcggcagcggcgagtggcaaacggtggacggcaactggcagcttaccctacagctaccggaatccggcggcgtagcggagcgcgtcgtcaccgcacttatgcgccgattagcccaacccgctccggggacgacggtcgcacctgaaaccattgcgattcgccagctactgcaacaattacctgaacatctgactatcgcccgggcgcaagagggttggctggcggcgatggtgggcggcagcggcaatctggcgcagcaggttgcccgtcagttgacgctgctcaataccccgatcaacgccgagcttcactcttcggcgggctgtcgccgcggtattcagcgtattccccacgccagctcggataacgcgctattagtttttattccattgcccgagggggcatcgctggccgcgctgcaactgctggcgctactttgcgagccgcaattcttccagcgtctgcgggtcgaacagcagattggctacgtggtgagctgccgttatcagcggatcgccgatcgcgatggcctgctgctggcgctgcagtcgccagatcgttccatcagtaacctgctacgctgctgtaaaacctttctccgccagttaacgctgggtgggcaagctgagttcagtcagttacaacaccagttggctgagcagcacggtctgccgcaggacgccagcgccaccgcactctccgccctgcaccagcgctatcatctaccggtagccacgccgcagaacgttaacgacctacagctggacgaggtcatcgcgctatggcgtgagataactcgccgtcgccgcagctggcgaatcctgtatagcgcgccgacgacgtccgccacttaa

SEQ ID NO: 154 - DNA, 1143 bp, Klebsiella sp.
  source 1..1143: mol_type = genomic DNA
gtgagccagaataaacccgccgttaatccgccgctgtggctactggcggagctgacctatcgctgtccgttgcagtgcccctactgctctaacccgctggactttgctcagcaggagaaagaactcaccaccgaacagtggattgaggtctttcgccaggcgcgggccatgggtagcgtgcagatgggtttctccggcggcgaaccgctgacgcgcaaagatctgccggagcttatccgcgccgcgcgcgacctcggtttttacaccaatctgattacgtcaggcattgggctcaccgagagcaaactcgacgccttcagcgaggccgggctggatcatatccagattagcttccaggccagcgatgaagtgcttaacgcggcgctggcgggcaacaaaaaagcgttccagcagaagctggcgatggcgaaagcggtaaaagcacgcgactacccgatggtgctgaactttgtcctccatcgccacaatatcgaccagatcgataaaatcatcgagctgtgtatcgagctggaagcggatgatgttgagctcgccacctgccagttttacggctgggcgttcctcaatcgtcaggggttactgccgacccgcgaacagatcgcccgagctgaacaggtcgttgctgattaccgccataaaatggcggctaacggtaatctgaccaatctgctctttgtcaccccggactactacgaagaacgccctaaaggctgtatgggcggctggggctcgatattcctcagcgtgaccccggaaggcaccgcgctgccgtgtcacagcgcgcgccagttaccggtcgagttcccatcggtgctggagcagagcctggaatcgatctggtatgactcgtttggcttcaaccgctaccgcggcttcgactggatgccggagccgtgccgttcctgtgatgaaaaagagaaagatttcggcggctgccgctgtcaggcctttatgctgaccggcaatgccgataacgctgacccggtgtgcagcaaatcgccgcaccaccataaaatccttgaagcgcggcgcgaagcggcctgtagcgacatcaaaatcagccagctgcagttccgtaaccgcacccgttcacagctgatctataaaacccgggaattgtga

SEQ ID NO: 155 - DNA, 279 bp, Klebsiella sp.
  source 1..279: mol_type = genomic DNA
atgcaaaaaagcgcaatcatcgcctttcgtcgcggctaccgcctgcagtgggaagctgctcaggatagccacgtgatcctctatccggaaggaatggcgaaactaaatgagaccgccgccgcgatcctcgaactggtcgatggtcaacgcgacgcggcaaaaataatcgccgaactcaacgcccgtttcccggaagccggcggcgtcgatgacgacgtcatcgaattcctgcagatagcttaccaacagaagtggattatcttccgtgagccagaataa

SEQ ID NO: 156 - DNA, 756 bp, Klebsiella sp.
  source 1..756: mol_type = genomic DNA
atgcagattcgcgaaaccctatcgccacaggcgtttgaacaagcgctacgggaaaaaggcgcctactaccatatccatcatccgtaccatattgcgatgcataacggcgaggccagccgcgaacaaatccagggctgggtggcgaaccgtttttactaccagaccaatatcccgctaaaagacgcggcgattatggcaaactgccccgatccacacacccgacgcaaatgggtgcagcggatcctcgaccacgatggcagtaacggccaggaaggcggtattgaagcctggctgcagttgggtgaagccgtggggctaagccgtgatgagttactcagcgagcgccacgtgctgcccggggtgcgttttgccgtcgacgcctacgttaattttgcccggcgggccaactggcaggaagcggcgtgcagttcgctgaccgaactgtttgccccgcaaatccatcagtcgcgcctcgacagctggccgcagcactacccatggatcaaagaagaaggctatttctacttccgcagccgcttaagccaggccaatcgcgacgtcgaacatgggctggagctggcgaagatctactgcgatagcgcggaaaagcagaaccggatgctggagatcctgcagtttaagctcgacattctgtggtcgatgctggatgccatgaccatggcctatgcgttgcagcgcccgccctatcatacggtcaccgacaaggcggcctggcacacgacccgactggtataa

SEQ ID NO: 157 - DNA, 918 bp, Klebsiella sp.
  source 1..918: mol_type = genomic DNA
atgtttataaaagtcctcggttccgccgctggcggaggttttccgcagtggaattgtaactgcgccaactgtcaggggctgcgcaatggcacaattcaggccaccgcccgcacccagtcttcgattatcgttagcgataacggcaaagagtgggtgctgtgtaacgcctctccggatatcagccagcaaattgcccacacgccagagttaaacaaacaaggcgtgctgcgcggcacgcatattggcggcattatccttaccgatagccagattgaccatactaccgggctgctgagcctacgcgaagggtgtccccatcaggtctggtgcacgccggaggtacatgaggatctcagcacgggttttcccatttttactatgttacggcactggaacggcggcctgattcaccatcctgtcaccccgctgaatagttttaccgttgacgcctgtcccgacctgcagtttaccgccgtccccatcgccagcaatgcgccgccctactcgccgtttcgcgataggccgctgccgggccataacgtggcgctgtttatcgaaaaccgccgcaacggccagacgctattctatgcgccggggcttggcgaaccggacgaagcgctcttgccgtggctgaaaaaagcggactgtctgctgattgacggcaccgtatggcaggataatgaactgcaggctgccggcgtcgggcgcaataccggtcgtgatatgggccatctggcgcttggcgatgaacacggcatgatggcgctgctggcctcactcccggcgaagcgcaagattctgattcatatcaacaatactaatccaatccttaacgagcagtcgccgcagcgtaacgccctgacgcaacagggaattgaagtgagctgggacggaatgcctatcacgcttcaggactaa

SEQ ID NO: 158 - DNA, 1263 bp, Klebsiella sp.
  source 1..1263: mol_type = genomic DNA
atgaaaaaaagcaccaccattccattaacgccattacagaaccaggggcgctatctgcccgcctataaacgccgctatcacatgatggcgaaacccagcggctccacctgcaacctggactgtcaatactgtttttatctgcataaagagcagcttttacatcagccgcacgacaaaggaatgagcgacgaggtactggagaatttcattcgtcagtacatccaaagccaggacggcgaagagattattttctcctggcagggcggcgagccaaccctgatgggactggagtttttcgaaaaggtcgttgagctgcaaaaaaaatatcagccgaagcaccagcgcattgagaacgatctgcagaccaatggcgtgctgattaacgataagtgggccgccttcttgaaggcgcataacttcctggtcggggtatcgatcgatggcccgcgcgagatccacgatcgcttccgcgtgacgcgcagcggcaagccgaccttcgataaggttatggaagggatcgcggcgctgaagcgccacggcgtgccgttcaacgcgctggcagtcgttaaccgggtgaatgcccgtttcccgcgcgaggtatatcgttttcttacccgcgaactcggcgccacctatatccagtttacgccctgcgtagaggccgccgagtttaaaactaccgcgccgcagttctggcgcgaagagacgatccccatcaccggcagccgccgggcgaaaccgggggatctggattcaatcgtcaccgactggtcggtggatccggacgattgggggcattttctgaccgaagcttttgatgagtgggtgacctgtgatctgggccgcgtgcaggtaaatttgttcgaaaccgccgtggtgcagactatggggcttccatcccagttatgcatcaccgcgccgttttgcggcaaggcgctggcgattgagaaaaacggcgacgtctactcctgcgatcactacgtctacccggaatataagcttggcaatattaaggcgcataagctggcgcatatggtcttttccgagcggcagaaagtgtttggcatgggcaagaaagagacgctgcccgcctactgcaaaagctgcccgcatctgaatttatgctggggcgaatgcccgaaaaaccgcatcgtgcgcgccccggacggcgaagaagggctcaattatttgtgtcccggctttcgccatttttacgcgacggttaaaccgacgcttgagaaaattgccgccatgctgaagtag

SEQ ID NO: 159 - DNA, 1296 bp, Klebsiella sp.
  source 1..1296: mol_type = genomic DNA
atgaaacacagcaccaccgtgcctgttaacgcattaccttcggcagaaaagtacgcgggtaacggccccgcctaccaacgtcgctttcatgtgatggcgaaacccagcggctccgcctgtaatctcgattgtagctactgcttttacctgcacaaagaacacttactccagcaggaaaagcgcagctacatgagcgatgaaacgctggaaaactttatccgccagtacatcgatggacaggatggcgaacaggtggtcttctcctggcagggcggcgaaccgaccctaatgggactggagtttttccacaaagtggtgaaattccaacagcaatataaaaagcccggccagcggatcgaaaacgatctgcaaaccaacggtatcctgattaacgatgcctgggctgaattcctcaaaacgaatcatttccttgtcggcctgtcgattgacggcccgcgagagttgcacgaccgttatcgcatcacccgcagcggcaaaccgacatttgataaagtgatggccggcgtcgacgccctgaagcgccacggcgttccattcaatgcgctggtgaccatcaaccgtaccaacgcccgtttcccattagaggtttaccgctttgttacccgcgaactgggggcgacctatgtgcagtttaacccctgcgtcgaaccggtggactttacccagaccgcgccccatttctggcgtgatgacactatccccaccgtcggcagccgccgcgcgcgccccggcgatttagactccatcgtcaccgactggtcggtcgacccggacgactggggacgcttcctgatagccaccttcgaagagtgggtgaacaacgatcttggccgcgtacaggtcaatctgttcgaaaccgccgtcgcccagatgatgggtctgccggcgcaaatttgcaccaccgcggagttttgcggcaaaggactcgcggtcgagaaaaacggcgatgttttctcctgcgatcactacgtctatccggagtatcaaatcggcaatatcgccgataacagcctggcgcgaatggctttctccgaacgtcagcaggctttcggtatgggtaaatgcgacacgctgccccagcagtgcaagcagtgcccttacctgaagctttgccacggcgagtgcccgaaaaaccgtctggtgctcactaccgacggcgaagccggtttgaactatctctgcccgggcattaaagcctttttcaactatgccgagccgattctggcgggcatcgtcacgctggtgaaacgcgattttaaaggagttagccgatga

SEQ ID NO: 160 - DNA, 921 bp, Klebsiella sp.
  source 1..921: mol_type = genomic DNA
atgagcactttaggtcatcaatacgataactccctggtatccaacgcctttggtttcctgcgtctgccgatgaactttatgccgtatgaaagcgatgcggattgggtcatcaccggcgtgccgtttgatatggcgacctccgggcgcgcgggcggccgtcatggcccggcagcgatccgtcaggtgtcaaccaacctcgcctgggagcacaaccgtttcccatggaacttcgacatgcgcgaacgcctgaacgtcgtggactgcggcgatctggtctacgccttcggcgacgcccgcgaaatgagcgagaaactgcaggcgcacgcggagaaactgctggcggccggtaaacgcatgctctccttcggcggcgaccatttcgtcaccctgccgctgctgcgcgcccacgccaagcatttcggtaaaatggcgctggtacacttcgatgcccacaccgatacctacgccaacggctgtgaattcgaccacggcaccatgttctacaccgcgccgaacgaagggcttatcgatccgaaccactccgtgcagatcggtattcgtaccgaattcgataaagataacggttttaccgtgcttgatgcgggccaggttaacgaccgcagcgtcgatgacatcatcgctcaggtgaagcagatcgtcggcgatatgccggtgtacctgaccttcgatatcgactgcctggatccggcatttgcgccaggtactggtacgccggtgatcggtggactcacctccgaccgtgcgatcaaactggtgcgcggcctgaaggatctgaacatcgtcgggatggacgtggttgaagtcgccccggcctatgaccagtccgaaatcaccgccctggcggcggcgactctggcgctggagatgctttatattcaggcggcgaagaaaggcgactaa

SEQ ID NO: 161 - DNA, 2886 bp, Klebsiella sp.
  source 1..2886: mol_type = genomic DNA
atgccccgcagtttatggttcaaagttcttgttgtagtggtcgccctttgggcgccgttaagtcaggcagacaccggatggcaaccgattcaggaaaccatccgtaaaagcgaaaaagatccccgtcactatcaggctattcgcctgcaaaacggcatggtcgtgctgctggtatccgatccgcaggcggtaaaatcgctgtcggcgctggtggtgccggtgggttcattacaggatccggccgaccatcaagggctggcgcactatctcgagcatatgaccttaatgggctcgcagaagtacccgcagcctgacagtcttgctgaattcctcaaaatgcacggcggcagtcataatgccagcacggcgccgtatcgcaccgcgttctaccttgaagtggaaaacgatgctctcaacggcgctgtcgatcggctggccgatgccattgccgcgccgctgctggataaaaaatatgccgaacgcgaacgtaacgcggtcaacgccgaattgactatggcgcgtacccgggacgggatgcgcatggcgcaggtgagcgccgaaaccatcaaccctgcgcatccggcttcacaattctccggcggtaacctcgatacactgagcgataagcctggcagcccggtgcttgatgcgctacacgctttccgcgaccgctggtactcggcaaacctgatgaaggcagttatctacagcaataagccgctgcctgagttggcaagcattgccgccgcgacctacgggcgggtgcctaatcatgacatcagcaagccggagattaccgtaccggtcgtgaccgatgcgcagaagggcattgtgattcactacgtgccggcgatgccgcgtaaggtgctgcgcgttgaattccgcatcgataacaacagcgctcagttccgcagcaagaccgatgagctggtgacctatatgattggcaaccgcagccccggcactttatccgactggctgcagaagcaggggctggcggaagggattcgcgcggattccgatccggcggtcaacggtaatagcggcgtgctggcgatctccgctaccctcaccgataaaggcctggcgcaccgcgatgaagttaccgccgctattttcagctacctgaatttgctgcgtacacagggtattgataagcgctactttgatgaattagcccacgtgctggagctcgatttccgttatccgtcgattaaccgcgatatggattatgtggagtggttggccgataccatgattcgcgtgccggtagagcacgtactggacgtggttaatatcgccgaccgctatgatgcccaggcgatcaaagaccgtctggcgatgatgaccccgcaaaatgcgcgtatctggtacatcagcccgaatgagccgcacaacaaaaccgcttatttcgtcaacgcgccgtaccaggtcgataaaattagcgcccggaccttcaccgactggcagcaaaaatctgcggccatcaagctgcagctgccgacgctgaacccgtatattccggatgatttcacgctgattaagagcgataaggcctacccgcatccggagcttatcgtcgatgagccgacgctgcgcgtggtgtacgcgccaagccagtactttgccagtgaaccgaaggccgacgtctcgctggtgctacgtaacccgcaggcgatggacagcgcgcgccgtcaggtgatgttcgcgttgaatgattacctggccggtatcgccctcgatcagcttagcaatcaggccgcggtaggcgggataagcttctcgaccggcgccaacaacggtctgatggttaatgccaacggctatacccagcgtctgccgcagctctttaccgcgctgctggacggctactttagctatacgccgaccgaagagcagcttgagcaagcgaagtcctggtatgcccagatgatggattcggcggacaaaggcaaagcctatgaccaggcgattatgccgattcagatgctgtcgcaggtaccctattttgagcgtaaaacacgtcgcgatttactaccgtcaatcactctgaaagaggtgatcaactaccgcgataacctgaaagcaagagggcggccggagctgctggtcatcggtaacatgagcgccagacagtccaccgatctagcccgccagatccaaaaacagcttggcgccgatggcaacgagtggtgtcgcaacaaagacgtgctgatcgacagcaaacagctggcgatgtttgagaaagcaggcagcagcaccgactccgcgctggcggcggttttcgcgccgccaaatgtcgatgaatacagcagcatggccgccagttcgctgttagggcagattattcagccctggttctacaaccagttacgtactgaagaacaactcggctatgcggtattcgccttctcaatgaatgtgggccgccagtgggggatgggcttcctgctacagagcagcgataaacagccggccttcctctggcagcgcttccaggccttcttcccgacggcggaagccaaactgcgggcgatgaaaccggaagagtttgcccagatccagcaggcggcgattagccagatgctgcaggcgccgcaaacgttgagcgaagaagcatcaaagctgagcaaagatttcgatcgcggtaatatgcgcttcgattcacgtgataaagtagtggctcagatgaaactgctgacgccgcaaaaacttgccgacttcttccatcagacggtggtggatccgcaaggcatggcactgttatcccaggtttccggcagccagaacggcaagacggaatacgccgcccctgaaggcggtaaggtatgggaaagcgtcagcgcattgcaaaaatccttacccctgatgcgagagaatgaatga

SEQ ID NO: 162 - DNA, 1416 bp, Klebsiella sp.
  source 1..1416: mol_type = genomic DNA
gtgaaattatctgccctctttattgccctgttaccgctgctggctcccccggttattcatgcgcaaaccacttcttcgccggtgctggaaaatcgcgcggcgcagggcgatatcacgaccccaggcggggcgcgccgtttaacgggcgatcagaccgaagcgctgcgcgcttcgttaatcaataagccggcgaaaaatattattttgctcattggcgatggcatgggtgattcagaaattaccgccgcacgaaattatgccgaaggcgccggcggtttctttaaaggtattgatgccctgccgctgacgggccaatacacccattattctctggataaaaaaaccggcaagccggattacgtgacggattccgccgcgtcggcaaccgcgtggaccaccggcgtgaaaagctataacggcgcgctgggcgttgatatccacgaaaaagatcatcagaccatccttgagctggcgaaagccgcgggtcttgccaccggcaatgtctcgacggctgaactgcaggatgcgacgccggcggcgcaggtggcgcatgtgacctcgcgtaaatgctatggccctggcgtgaccagcgaaaaatgcgccagcaacgcgctggaaaaaggcggcaagggctctatcaccgaacaactgctgaatgcccgtccggatgtctctttaggcggcggggcgaagacctttgctgaaacggcgaccgcaggggagtggcaggggaaaaccctgcatgagcaggcggtcgctcgcggctatcagattgtgaccgacgccgcttcactcgccgctatcagcgaagcaaatcagggtaaaccgctgctcggtctgttctccgacggcaatatgccggtgcggtgggaaggcccgaaagcctcttatcacggcaatatcgacaaaccgccggtaacctgtacgccaaacccgaaacgcgatgcttccgtaccgacgctggcgcagatgaccgaaaaagcgattgatctcctgagccgcaacgagaaaggtttcttcctgcaggttgagggggcgtctatcgataaacaggaccacgcggcaaacccttgcggccagattggcgaaaccgtcgacctcgatgaagcggtacagaaagcgctggaatttgccaaaaaagagggcaataccctggtgatcgtcaccgccgatcacgcccactccagccagattatcccggcagacacgaaagcgccgggcctgacccaggcgctgacgaccaaagatggcgcggtaatggtgatgagctatggcaactctgaagaagagtcgatggagcacaccggcacccaactgcgtattgccgcgtacggaccgcatgcgggcaacgtggtgggtctcaccgaccagaccgatctgttttacaccatgaaagacgcgctgggcctgaaataa

SEQ ID NO: 163 - DNA, 1296 bp, Klebsiella sp.
  source 1..1296: mol_type = genomic DNA
gtgctggaacggctgtcatggaaaaggctggcgcttgagcttttcttatgttgtatcccggccctgatcctcggggcgtttttcggtcatctgccgtggtttttactggcggcggtgaccggattgcttatttggcatttctggaatttgttacgtctgtcgtggtggctgtgggtcgatcgcagtatgacgccgccgccgggaagcggcagttgggaaccgctgctgtatggtctgcatcaaatgcagatgcgtaataaaaaacgtcgccgcgagctggggagcctcatcaagcgctttcgcagcggggcggaatccttacccgatgcggtggtgctgaccaccgaagagggcgccatcttctggtgtaacggcctggcgcaacaaatccttaatctacgctggccggacgatagcgggcagaatatcctcaacctgctgcgctacccagagtttgctaactatctgaaaaaccgtgatttcaccaagccccttaatctggtgctcaataacgcccgtcatctggaaattcgcgtgatgccttatagcgataaacagtggttgatggtggcgcgcgatgtcacgcaaatgcaccagttggaaggcgcgcggcgcaactttttcgccaacgtcagccacgagctgcgcacgccgctcaccgtactgcagggttatctcgagatgatgcaggagcaggtgctggaagggccgacgcgggaaaaagcgctgcataccatgcgtgagcaaacgcagcggatggaagggttggtgaagcagttgctgacgctctcgcgtattgaagccgcgccggtgctggcgatgaatgataaaatcgacgtcccgatgatgctgcgggtggttgagcgcgaggcgcaaaccctcagccagggtaagcatcagctgcactttagcgtcgatgaaacgctccaggtgatgggcaacgaagagcagttgcgcagcgcgatttccaatctggtgtacaacgccgttaaccatacgccagcgggaactgagattcacgtcagctggcagcgggcgccacacggcgcgctgttcagcgttgaagacaacggtccgggcattgcgcctgaacatttaccccggctaaccgagcggttctaccgcgtcgacaaggcgcgctcccggcaaaccggcggcagcggtttagggctggctatcgtgaaacatgcggttagccatcacgaaagccggctggaaatcgaaagtacggtcgggaagggaacacgctttagcttcctgctgccggaacgtttaattgccaaaaatggcgcctga

SEQ ID NO: 164 - DNA, 3882 bp, Klebsiella sp.
  source 1..3882: mol_type = genomic DNA
atgagtacacgtttaccgctggttgcggcgcagccgggaatttggatggctgaacgcctctccacgctacccggcgcctggagcgttgcccactatgttgaactgcgcggcaatcttgatccggcgctgctgagtaaggcgatcgtcgccgggcttaagcaggccgataccctgagcatgcgcttttgcgaagataatggcgaagcgtggcagtgggttgatgacgcgcgcgagtttgccgagccgcaaagcgttgacctgcgccagcaggccgatccgcatatgtccgcgctgacgctgatgcaaagcgatctcgggcagaatctgcgcgtcgacagcggtaatccgctggtctgccatcagctcatgcgcgttggcgacgactgctggtactggtatcagcgctatcaccatttactggtcgatggttttagtttcccggccattacccgccagatcgccgctatttatcgtgcctggcaacgcggcgaagagacgccggattcaccgtttacgccgttcgccgaagtggtggaagagtatcagcgctactacggcagcgaggcgtggcagcgcgataaagccttctggcaggcgcagcgtcaggcgttgccgtctccggcctcgctatcagacgcgccgctggccgggcgggcgaccagcagcgatatctggcgcctgaaactggaggccgatccggctgtcttcagccagctcgcggccagcgcgccgcagtgccagcgcgccgatctggcgttggcgctgacgacgttgtggctggggcggttgtgcggccgaatggattacgcggctggctttatctttatgcgccgcatgggatcggcggcgctgaccgccaccggcccggtactcaacgtactgccgctggcggtgaatatcgacgcgcaggagacgctggcgcaactggcgacgcgtctctccggacaactgaaaaaaatgcgtcgccatcagcgctacgatgccgagcaaattgtccgcgatagcggtaaagcggcgggcgacgaaccgctgtttggcccggtgctgaacatcaaagtctttgattaccagttggatatcgacgacgtgcaggcgcagacgcataccctcgccaccggcccggtgaatgacctcgagctggcactgttcccggacgaacgcggcggtctgtcgctggagatcctcgccaataaagcgcgttatgatgaggcgacgctgaaccgccacgtgtcgcggctgaccgcgctgctggcgcagttcgccgccaacccggcgctacgctgcggcgaggcggagatgctgtcggcggaagaaggcgcgcagctggcattgatcaacaataccgcgatgccgctgccgaccactaccctcagcgcactggtggcggaacaggcgcggaaaacgccggatgccccggcgctggcggatgccaactggcgctttagctaccgcgaaatgcgccagcaggtggtggcgctggccaacctgctgcggcaacgcggcgtgaagccgggggatagcgtggcggttgccttaccgcgctcggtgttcctgacgctggcgctgcacggcattgtcgaagccggcgccgcctggctgccgctggacaccggctacccggatgaccgtctgcggatgatgctggaagatgcgcggccatcgctgctgattacctctgacgatcaactggcccgttttagcgatattccgggactgaccagcttgtgttatgagcagccgctggctgctgaagatgatacgccgctggcgctgtcaaaaccggaacataccgcgtatatcatcttcacctccggttccaccgggcggccgaaaggggtgatggtcgggcagaccgccatcgtgaaccgcctgctgtggatgcaaaatcactatccgttaaccgccgccgatgtggtggcgcagaagacgccgtgcagctttgatgtctcggtatgggagttctggtggccgtttatgaccggcgcccagctggtgatggcggcgccggaggcgcatcgcgatccacaggcgatgcagcggttcttcacggattatggcgtgaccaccacccactttgttccgtcgatgctggcggcgtttgtcgcctcgctggatagcgataatgtgtcttcctgccggacgctaaaacgcgttttctgtagcggcgaagcgctgccgacggaactgtgtcgcgaatgggagcgcttaaccggcgcgccattgcataacctgtacgggccgacggaagcggcggtggatgtcagctggtatcccgcctgcgggccagagctggcggcggtaaccggcaacagcgtgcctatcggctggccggtatggaacaccggattacgtattcttgatgctgcgatgcgccctgtgccgccgggcgtggcgggtgatttgtacttaacgggcattcagctggcacagggatatatggggcgccctgatctcaccgccagccgctttattgccgacccgtttgcccccggcgagcgcatgtaccgcaccggcgatgtggcgcgctggctggataacggggcggtagagtatcttggccgcagcgacgatcagttgaaaattcgcggccagcgcattgagcttggcgagatcgaccgggtgatggcggcgctgccggacgtcgcgcaggcggtgtgccacgcctgcgtcttcaatcaggcggcggccaccggcggcgatgcccgccagctcgtcggttatctggtgagcgagtccgaccagccgctggacgtggcggcgctgaaggcgcgtcttagcgaacagctgccgccgcacatggtgccggtggtgttaatccagctggaatccttcccgctcagcgccaacggtaagctggatcgtaaagcgctaccgctcccctccttaagcagcgagcgcagcggtcgcccgccgcaatcggctaccgaaatggccgttgccgcggcgttcagccagctactgggctgcgaggtaaatgacatcgacgccgatttcttcgccctcggtggccattcgctgctggcgatgcggctggcggcgcagcttagccgcgagctggcgcggcaggtgacgccggggcaggtgatggtcgcttcgaccgtcggcaagctgagcgcgctgctggcttccgacctgagcgacgagcaggcgcagcgcctgggcttcgatgcgctgttgccgctgcgcgagagcgacggtccgacgctgttctgcttccatccggcctccggttttgcctggcagttcagcgtgctggcgcgttatcttaacccgcgctggtcaattaccggtattcagtcgccgcgtccgacggggccgatggcttccgccgccaacctcgacgaagtctgcgagcaccatctgcgcacgcttttagcgcagcagccgcatgggccttactatctgtttggttattcgctcggcggaacgctggcgcaggggattgccgcccgtttacgccagcgcggcgaagaggtggcgttcctcggcctgctggatacctggccgccggagacgcaaaactgggcggagaaagaggcgaacggtctggatcccgaagtgctggcggaaatcgcccgcgagcgcgaagcgtttctcgccgctcagcaggggcaggcttccggcgagctgttcagcgcgatcgaaggtaactacgccgatgcggtgcgcctgttgacgaccgcgcacagcgcgaagtttgacggcaaggcaacgctatttgtggcggagaaaacgcgccagaaagggatggatccgcaggtcgcatgggggccatgggttggcgagctggaggtgttcagccagaactgcgcccacgtcgagattatctcgccgcaggcctttgaagcgataggcccggtagtgcgggagattctggggtaa

SEQ ID NO: 165 - DNA, 1176 bp, Klebsiella sp.
  source 1..1176: mol_type = genomic DNA
atggaaacgtcactggctgaggatgtacaggaaaaaacgcagaccctgtcgccgcaaagcttcttctttatgtcgccgtaccgcagcttcagtaccaccggctgttttagccgtttttcccagcctgccgtcggcggtgattcgctgaacggcgagttccagcagaaaatggcggccgcttttgccgaagcccgtgcggcggggatccgcaagccggtcatggtcggggcgattccgttcgacaccaatcagccttccgagctctatattcccgaacgttgggaaaccttctcccgtaccgacaaacagcagtccgcgcgttacgcggcgccgctcaaagctatggatgttgtcgatcgtcaggagatcccggaacaggacgagtttttggcgatggtggaacgcgccgccgcgctgacggcgactccggaagtcgacaaagtggtgctctcaaggctgattgatattacgacccgcgaccgggtcgatagcggcgcgctgctggagcggctgatcgcccagaacccggcgagctttaacttccatgttccactctccaatggtgatgtgctgctgggcgccagcccggaactgctgctgcgtaaagaagggctgcacttcagttcgctgccgctggccggctccgcccgccgccagcctgacgatgtgctggaccgggaagcgggtaacaagctactggcgtcgggcaaagatcgccatgagcacgagctggtgacgcaggcgatgaagagcgtgcttggccctcgcagcagtcatctgtcgctaccggagtcgccgcagctgattaccaccccgacgctctggcatctggcgacgccgatcgaaggtacggcgctggcggaagaaaacgccatgtcgctggcctgcctactgcacccgaccccggcgctgagcggcttcccgcaccaggtggcgaagcggctgattgccgagctcgaaccgttcgatcgccaactgtttggcggcattgtcggctggtgcgatgacgaaggcaacggcgaatgggtggtgaccatccgctgcgcgcgcttacatcaacgctcgctacgcctgtttgccggcgcgggtatcgtcccggcctcttcgccgctgggcgaatggcgtgaaaccggcgtcaaactcaccaccatgctcaacgtatttggcttgaattaa

SEQ ID NO: 166 - DNA, 1608 bp, Klebsiella sp.
  source 1..1608: mol_type = genomic DNA
atgattgcatttacccgttggccggaagagtttgcggcccgctatcgtcaaaaaggctactggcaggatctgccgttaaccaatctgattactcgtcatgcggacaatgacgccgtggcgattatcgacggcgagcggcagattagctaccgccagtttaaccaactggtggataacctcgcgtcctccctgcagcatcagggactcaagcgcggagaaaccgcgctggtgcagcttggcaacgtcgccgagttctacattactttctttgcgctgctgcgcatcggcgtcgcaccggttaacgctttgtttagccatcagcgcagcgaacttaacgcctatgccgcgcagattaaaccggtgctgctgattgccgatcgcgagcacgcgctgtttgctgacgatagctttctccatggttttatcgctgagcatccttcgctgcgcgtcgcgctgctgcgtaacgatggcggcgagcgcgacctggcaacggaaattagccgcacggcggataactttgtcgccaatccgacgccggctgacgaagtggcttttttccagctctccggcggcagcaccgggacgccgaagcttattccgcgaacccataacgattacgactacagtatccgccgcagcaacgagatctgcggtattaacgccgacacgcgctatctcaacgcgctgccggcggcgcacaactatgcgatgagctcgccgggatcgctgggcgtctttctcgccggcggccgggtgatcctcgccgctgacccgaacgccacgttgtgtttcccgctgatcgaaaaacatcagattaacgtcgcgtcgctggtgccgccggcggtcagcctgtggctgcaggctattcatgaatggggcagcaacgcgcaactgcaatcgctgaagctgctgcaggttggcggagcgcgcctgtccgcgacgctcgccgcgcgtatcccggcggaaatcggctgccagctgcagcaggtgttcggcatggcggaagggctggttaactacacccgcctcaacgatagcccgcagcggattatcaacacccagggttgcccgatgtgtcctgacgacgaagtgtgggtggcggatgccgacggtaacccgctgccgcgcggcgaagtgggccgtctgatgacccgcggcccttataccttccgcggctattacaacagcccgcaacataacgctgaagcctttgatgccgatggattttactgctccggcgatctgatttcgatcgatgaagatggctatatcaccgtccagggccgggaaaaagatcagatcaaccgcggtggcgagaagatcgccgctgaagagatagaaaacctgctgctgcgccatgaggcggtgatccacgccgcgctggttagcattgaagacaatctgctgggcgagaagagctgcgcttatctggtggtgacatcgccgctgcgcgccgtcgcggtgcgccgcttcctgcgcgagcagggcgtggcggaatttaaattgccggatcgcgtggagtgcgttgccgcgctgccgctgacgccggtgggcaaagtcgataaaaaacaattacgtcagtggctggccgaaggcaagctgggctga

SEQ ID NO: 167 - DNA, 852 bp, Klebsiella sp.
  source 1..852: mol_type = genomic DNA
atggcaatccccaaattacaggcttacgcgctgccggaagccagcgatattccggccaacaaagttaactgggcctttgagccgtcgcgcgccgcgctgctgatccatgatatgcaggagtatttcctcaacttctggggcgaaaacagcgcgatgatggaaaaagtggtggccaatatcgccgccctgcgcgacttctgcaaacaaaacggcattccggtgtactacaccgcccagccgaaagagcagagcgatgaagaccgcgccctgctgaatgatatgtgggggccggggttgactcgctcgccggaacagcagcaggtgattgccgcgctggcgccggatgagcaggatacggtgctggtgaaatggcgctacagcgcgtttcatcgctcaccgcttgaagagatgctgaaagagaccggccgcgaccagctgatcatcaccggcgtttacgcccatatcggctgtatgaccaccgccaccgacgcttttatgcgcgatatcaaaccgttctttgtcgccgacgcgctggcagatttcagccgcgaagagcatttgatggcgctgaaatacgtcgccggccgttctggacgcgtggtgatgaccgaagaattgttgccgctgccggcctccaaagcggcgctgcgcgcgctaattctgccgctgctcgacgaatccgacgaaccgctggatgatgaaaacctgatcgactacggtctggattcggtgcgtatgatggcgctggccgcccgctggcgcaaagtacacggcgatatcgacttcgtgatgctggcgaaaaacccgaccatcgacgcctggtgggcgctgctctcccgcgaggtgaaataa

SEQ ID NO: 168 - DNA, 1689 bp, Klebsiella sp.
  source 1..1689: mol_type = genomic DNA
atggcaagttcgggcaccacatcaaacacaatgcgctttaccggcgcgcagctggttgttcatttactggaacgccagggtatcactatggtcagcggcattccgggcggctccatcctgcctatctatgatgccttgagccaaagcacgcagatccgccacatcctggcgcgccacgagcagggggcgggttttatcgctcaggggatggcgcgtaccgaaggtaaacccgcagtctgcatggcctgtagcggccctggcgccaccaacctgattaccgccatcgccgatgcccgcctcgattctattccgctggtctgcatcaccgggcaggttccggcctcaatgatcggcaccgatgccttccaggaagtcgatacctacggcatctctatccccatcaccaagcataactacctggtgcgcgacatcgccgagctgccgcaggtgatgagcgacgccttccgtattgcccagtccgggcgccccgggccggtgtggatagatattcctaaggacgtacaggcggcaaccattgaactggaaacgctgccggagccaggcgagcgcgccccggcaccagcattcgcgcctgaaagcgtgcgtgaagcggcggcaatgatcaacgcggcgaaacgcccggtactgtatctgggcggcggcgtgattaacgcgcctgagccgattcgggacctggcggaaaaagccaacctgccgaccaccatgaccttaatggcgctgggtatgttgccgaaggcgcatcctttgtcgctgggcatgctggggatgcacggcgcgcgcagcactaactttattctgcaagaagctgatttactgattgttttaggcgcgcgttttgatgaccgggcgattggcaagaccgagcagttctgcccgaacgcgaagattattcacgtcgatatcgaccgctctgagcttggcaaaattaagcaaccgcacgtagcgattcagggcgatgtggcggaggtcttagcccagcttattccgcagattgaggcgcagccgcgtgatgagtggcgccagctggtggctgatttacaaagagaattcccctgcgccatcccgcaggaaagcgatccgctctcccattacggcctgattaacgccgtggccgcctgcgttgatgatgaggcgatcatcaccaccgatgtgggtcagcatcagatgtggacggcgcaggcctatccgctcaaccgtccgcgccagtggctgacttccggcggtctcggcaccatgggcttcggcctgccggcggccatcggcgcggcgctggccaatccgcagcgtaaggttatttgtttctccggtgacggcagcctgatgatgaatattcaggagatggccaccgctgccgaaaatcagctggatgtaaaaattatcctgatgaacaacgaagcgctggggctggtgcatcagcagcagagcctgttctatcagcagggcgtcttcgccgcgacctatccggggatgattaactttatgcatatagctgccggttttggtctgcaaacctgtgatttaaataacgaatccgatccgcaggcggcgctgcaggcgattatcaaccgcccaggtccggcgttgatccacgtgcgtatcgacgcgcagcaaaaagtgtatccgatggtaccgccgggtgcggccaatactgagatggtgggggaataa

SEQ ID NO: 169 - DNA, 288 bp, Klebsiella sp.
  source 1..288: mol_type = genomic DNA
atgcaaaagcaacacgataacgtcattctggaactcaccgtccgcaaccacccaggtgtgatgacccacgtctgcgggctatttgcccggcgcgcgtttaacgttgaaggcattctctgcttgccgatccagggcagcgagtacagccgcatctggctgctggtaaatgatgaccagcggctggggcagatgattagccagattgaaaagctggaagatgtcaccaatgtggcgcgtaaccagtccgatcccaccatgtttaacaaaattgcggtgttcttcgaatag

SEQ ID NO: 170 - DNA, 2544 bp, Klebsiella sp.
  source 1..2544: mol_type = genomic DNA
atgagcaaagtcagaatcgctattatcggtaacggcatggtcggccatcgctttatcgaagagcttcttgataaggcgcctgccggacaattcgacattaccgtgttctgtgaagagccgcgtatcgcctatgaccgtgtccacctgtcgtcttacttctcccatcacactgcggaagagctgtcgctggtacgcgaaggtttctatgagaaacacggcgtcaaggtactggtcggcgaacgcgcgattaccatcaaccgtcaggaaaaggtgatccactccagcgctggccgtaccgttttctacgacaagctgattatggcgactggctcatacccgtggatcccgcccattaaaggcgcggaaacccaggattgcttcgtctatcgtactattgaagatcttaacgccatcgaatcctgcgcccgtcgcagcaaacgcggcgcggtggtcggcggcggtctgctcggcctggaagcggctggcgcgctgaaaaatctcggcgtggaaacccacgtgatcgagtttgcgccgatgctgatggccgagcagctcgaccagatgggcggcgagcagctgaagcgtaagattgaaagcatgggcgtgaaggttcacaccagcaaaaataccaaagagatcgttcagcagggcaccgaggcacgcaaaacgatgcgcttcgccgacggtagcgaactgcaggtcgatttcatcgtcttctctaccggtatccgtccgcgcgacaagctggctacccagtgcggtctcgccgtcgcccaacgcggcggcatcatggtcaacgatagctgccagacctccgatccggatatctacgctatcggcgaatgcgccagctggaataaccgcgtatacggtcttgtcgccccgggctacaaaatggcgcaggttaccgttgaccatatcctcggcaacgacaatctgttcaccggcgccgacctcagcgccaagctgaagctgctcggcgtggacgttggcggtatcggcgacgcccacgggcgcacgccgggcgcgcgtagctacgtctatctcgacgaaagcaaagaggtctacaaacggctcattgtcagcgaagataacaaaaccctgcttggcgcggtactggtcggcgacaccagcgattacggcaacctgctgcagctggtgctgaatgccatcgagctgccggaaaacccggattcgctgatcctcccggcccacgccggcagcggcaagccgtccatcggcgtggataaactgccggacagcgcgcagatttgttcctgcttcgacgtcagcaaaggcgacctgatcgccgctatcaataaaggctgccacaccgtggcggcgctgaaagcggaaaccaaagccggaaccggctgcggcggctgtattccgctggtgacgcaggtgctcaacgccgagctggcgaaacagggtatcgaagtgaacaacaatctgtgtgagcacttcgcctactctcgccaggagctgttccacctgatccgcgtcgaaggcatcaaaaccttcgacgaactgctggaaaaacatggtcagggctacggctgtgaagtctgtaagccgaccgtcggttccctgctggcctcctgctggaatgagtacatcctcaaaccacagcacacgccgctgcaggacaccaacgataacttcctcgccaacatccagaaagatggcacctactcggttatcccgcgctccgcaggcggcgaaattacgccagaaggcctggttgccgtcggtcgcatcgcgcgcgaatttaatctgtataccaaaatcaccggctcccagcgtatcggcctgttcggcgcgcaaaaagacgatctgccggaaatctggcgtcaactgattgaagccggcttcgaaaccggccatgcctacgccaaagcgttacgtatggcgaaaacctgcgtcggcagtacctggtgccgctacggcgtcggcgatagcgtcggcttcggcgtagaactggaaaaccgctataaaggtatccgtaccccgcacaaaatgaagttcggcgtctccggttgtacccgtgaatgcgcggaagcccagggtaaagacgttgggatcatcgccaccgagaaaggctggaacctgtacgtgtgcggtaacggcgggatgaaaccacgccacgccgatctgctggccgcggacctcgatcgcgatacgctcatcaaatatctcgaccgctttatgatgttctacatccgcaccgccgataagcttacccgtaccgcgccgtggctggacaacatggaaggcggtatcgactatctgcgcagcgtcatcatcgacgataagctgggcctgaacgatcaccttgaagaagagctggctcgcctgcgcgccgcttttgcctgcgaatggaccgagaccgtcaataacccggcggcgcagacgcgcttcaaacactttatcaacagcgatcagcgcgacccgaacgttcaggtggtgccggaacgtgaccagcatcgcccggccacgccctatgagcgcatcccggtcacgctggtggaggaaaacgtatga

SEQ ID NO: 171 - DNA, 891 bp, Klebsiella sp.
  source 1..891: mol_type = genomic DNA
gtgctagtgatattgggttatctcgtggtcctgggagcggtatttggcggctatttactggtgggcggccaccttggcgcgctgtaccagccggcggaattcctgattatcggcggcgccggcatcggcgcgtttatcgtcggcaacaacggaaaggccatcaaatccacgctgcgggccttgcccaaaatggtacgtcgctccaaatacagcaaggcgctgtatatggatctgatggcgttgctgttcctgctgctcgcaaaatcccgtcaacagggaatgctgtcgcttgagttcgatatcgacaatcctcaggaaagtgaaattttcgctaattatccgcgcatccttgccgataaccatctggtggagttcataaccgattatttacgcctgatggtgagcggcaacatgaatgcttttgaaattgaagcgctgatggacgaagagatcgaaaccttcgaacaggagagcgaagtgcccgccggcagcctggcgatggtcggcgactctctgccggcatttggtattgtcgcggcggtaatgggggtggtgcacgcgctggcctcggcggaccgtccggcggcagagctcggggcgcttatcgccaacgcgatggtggggactttcctcggcattctgctggcctatggatttatctcgccgctgtcgacgctgctgcggcaaaaaagcgccgagacggtcaagatgatgcagtgcatcaaggtgacattgctctccagcctcaacggctacgcgccgcaaatcgccgtcgaatttggccgcaaaacgttatacaccaccgagcgcccgtcgtttgtcgagcttgaagaacacgtacgccaggtgaaagcgcctgccgcgcaggcgacggaaagcgaagaagcatga

SEQ ID NO: 172 - DNA, 924 bp, Klebsiella sp.
  source 1..924: mol_type = genomic DNA
atgaagcataatcacccggtggttctggtcagaaagcgcaaatcacaccagccagcccatcacggcggttcgtggaagattgcctatgccgattttatgacggcgatgatggccttcttccttgtgatgtggctgctggcgatcgccagcccgcaggagctgacgcaaatagccgaatatttccgtacgccgctgaaagtcgccctcaccagcggcgataaaagcagctcggaaagcagcccgatccccggcggcggcgaagacccgacccgtgaagtcgggttagtcagcaagcagatcaataccgcggataaacgtgccgaagagctacggctaaacaaactgcgtgacaagctcgatcagttgattgagtcagacccgcgcctgaaggcgctacgtccgcatctgctgatcaacatgatggacgaaggactgcgcattcagattatcgatagccagaatcggccgatgtttaaaaccggtagcgctcaggtggaaagctatatgcgcgatatcctgcgagcgattgcgccaatcctcaatgatttgccgaacaagataagcctcgccgggcataccgatgacatcccctacgccagcggcgaacgtggttacagcaattgggaactgtcggcggatcgcgccaacgcctcgcgccgcgagctgatcgccggcgggctggcggaagggaaggtgctgcgggtggttggtatggcggcgaccatgagcctgaaacagcacggcgccgatgatgccatcaaccgccgcattaccgtactggtgcttaacaaagagacccagcagagcattgagcatgaaaatggcgaaagcaatgcgctggagattagccagccggatacgctgccaaagctggtcgcgcccgcgccggttaaccgcgattcacaccctgaggtgaccccatga

SEQ ID NO: 173 - DNA, 1959 bp, Klebsiella sp.
  source 1..1959: mol_type = genomic DNA
atgagcatggatattagcgctttttatcagactttttttgatgaagcagatgagctgctggcagacatggagcaacacctgctggagctggatccgcaggcccccgatatcgaaccgctaaacgctatttttcgcgcggcgcactcgatcaaaggcggcgcggcgacgtttggcttttccgtattgcaggaaaccacccatctgctggagaacctgctcgacggcgcccggcgcgaggagatgcgcttgagcaccgaaattatcaatctgtttctggaaaccaaagatattatgcaggagcaactggacgcctataaaacctcgcagcagcctgacgctgagagctttgactatatctgccaggcgctgcggcagctggcgctggaagcgcagcagcaggacgcgccagccgcgccgcccgtcgtggcccagcctgcgccgacggccgtcgccggcggtatgcgcgtgagtttaaccgggcttaaagccaatgaaattccgctgatgctggaagagcttggcaatctcggcgaggtacatgatccgcagcaaactgacaatagccttgaggtgaccctgctgaccaccgccagcgaagaagatatttgcgcggtgctctgtttcgttcttgagccggaacagatcagctttaccacgccgccaaccactgccgctaaaccattgccatcggccgaagttgtgccgccgccggtcgcccaaccgcagcctgctgtcgtcgagccgccgaaagcgccgagagcgaaggccagcgaatcgaccagtattcgcgtggcggtagagaaagttgaccagttgattaacctcgtcggcgaactggtgatcacccagtccatgctggcgcagcgctcaggtaacctggatccggtgacccatggcgatctgctcaacagcatgagccagctggaacgtaacgctcgcgacctgcaggaatcggtgatgtcgattcgcatgatgccgatggaatatgtattcagccgctacccgcggctcgtgcgcgacctggccggcaagctcaataagcaggtggaactgacgctgcagggcagctccaccgaacttgataagagcctgatcgagcgcattatcgacccgttaacccacctggtgcgcaatagcctcgaccacggtattgaagatccgcaaacccgtctggcggcaggtaagtctgaggtgggcaatctgattctttccgctgaacatcagggcggcaatatctgcatcgaagtgatcgatgacggcgccgggctcaatcgcgaaaaaattctcgccaaagcggcggcgcaggggctggcggtcagcgacagcatgagtgatgaagaggtcggaatgcttatttttgcgccgggcttttcgaccgcggaacaggtgaccgacgtctctggccgcggcgtcggcatggacgtcgtgaaacggaacatccaggagatgggcgggcacgttgaaatccattcccgcgcgggcaaagggacctcgattcgtattttgctgccgctgacgctcgctatcctcgacggcatgtcggtcaaggtcaatgaagaggtctttattctgccgctcaacgcggtcatggaatcgctgcaaccgcaggccgaagacctgcatccgatggccggcggcgagcggatgctgcaggttcgcggcgagtatctaccgctggtggagctctaccgggtgtttgatgtcgccggggcgaaaaccgaggccactcagggcatcgtggtgattctgcaaagcgccggccgccgctatgcgctgctggtggatcagctgatcggccagcaccaggtggtggtgaaaaacctggaaagcaattaccgcaaagtgccgggaatttccgcggcgacgatcctcggtgacggcagcgtggcgctgatcgtcgacgtgtcggcgctgcaaatgctcaatcgggaaaagctgctgagcgcagcggccgcataa

SEQ ID NO: 174 - DNA, 501 bp, Klebsiella sp.
  source 1..501: mol_type = genomic DNA
atggcaggattagcaaccgtcagcaaattggctggcgaaacggtaggtcaggagtttttaatctttaccctcggcaatgaagaatacggcatcgatattctgaaagtgcaggagatccgcggctatgaccaggtgacgcgtatcgccaacaccccggatttcatcaaaggcgtcaccaatctgcgcggggtgatcgtgccgattatcgacctgcgggtaaaattcgcccagcagggcgtctcttatgatgaaaacacggtggttatcgtgcttaacttcggccagcgggtggtggggattgtggtcgacggcgtctctgacgtgttgtctctcaccgccgaacagatccgcccggcgccggaattcgcggtaacgctggcgaccgagtatctcaccggtcttggcgcgctcggcgagcgtatgttgattctcgtcgatatcgaaaagctgctcagcagcgaagagatggcgctggtcgataacgtcgccaaaagccactaa

SEQ ID NO: 175 - DNA, 873 bp, Klebsiella sp.
  source 1..873: mol_type = genomic DNA
atgaagcagacgacatcaaccgcggcgcgtgaaagcggatcggcgctggcgcagatggttcagcgtctgccgctctccgacgcgcattttcgccgcatcagccagcttatctatcagcgtgccgggatcgtgctggcggcgcataagcgcgagatggtgtacaaccggctggtgcgccgtttgcgtctgctgggcattcatgatttcggcgactacctggcgctgctggaaagcgacccgcacagcgccgagtggcaggcgtttatcaatgcgctgaccaccaacctgaccgccttttttcgcgaggcgcaccactttccgattctggcggagcatgcgcgctcgcgtcccgggaactatagcgtgtggagcaccgccgcctcgaccggcgaagaaccttattccatcgccattacgctcggtgatgcgctcggggaacgtgcgggcagttgccaggtctgggccagcgatattgacacccaggtgctggagaaagcggaagcagggatttatcgccatgaagatctgcgcactctgacgccaatccagatgcagcgttattttctgcgcggcaccgggccgcatcaggggctggtgcgcgtgcgtcaggagctggcggcgcgggtcaacttccagccgctgaatctgctggcggcggagtgggcgctgccggggccgttcgacgcgattttttgccgcaacgtgatgatctatttcgataaaccgacgcaggaacgcatcctgcgccgctttgtccccttgcttaaaccgggggggctgttgtttgccggtcactccgagaatttcagccagatcagccgggatttctacctgcgtggacagaccgtgtatgggctggccaaggagaagtaa

SEQ ID NO: 176 - DNA, 1050 bp, Klebsiella sp.
  source 1..1050: mol_type = genomic DNA
atgagtaaaatcagagtgttatgtgttgatgattcggccctgatgcgccagttgatgaccgagatcgtcaatggccacgccgatatggagatggtggcggtggccccggacccgctggtcgcacgggatctgatcaaaaaatttaacccacaggtgctgacgctggacgtcgaaatgccgcgcatggacggcctcgatttcctcgaaaagctgatgcgcctgcggccgatgccggtggtgatggtttcatcgctgaccggtaaaggttcggaaattaccctgcgcgcgctggagctgggggcggtggatttcgtcaccaaaccgcagctcgggatccgcgaaggcatgctggcttacagcgaactgataggcgaaaagatccgtaccgccgcacgggcgcggctaccgcagcgcgccaatgaccagccgccggccattttaagccacggaccgctgctgagcagcgagaagctgatcgccattggcgcttccaccggcggcaccgaagcgatccgtcaggtgctacaaccgttgccggccaccagtccggcgctgctgatcacccagcatatgccgccgggatttacccgctcgtttgccgaacggttgaataagctgtgccagatcacggtgaaagaggcggaagagggcgaacgcgtgctcccgggacacgcctatattgcgcctggggatcgccatctggagctggcgcgtagcggcgctaactatcaggtcaagctgcacgacggcccggcggtaaatcgccatcgtccctcggtggatgtgctgtttcgttcggtggcccgccacgccgggcgcaatgcggtgggggtgatcctcaccggcatgggcaacgacggcgcgcaggggatgctggagatgcatcgcgccggggcctataccctggcgcagagtgaggcgagctgcgtggtgttcggcatgccgcgcgaggccatcgccagcggcggggtcaacgaagtggttgaactggagcgcatgagccaacgcatgctggcgcagatagccggcggccaggcgctgcgaatttaa

SEQ ID NO: 177 - DNA, 390 bp, Klebsiella sp.
  source 1..390: mol_type = genomic DNA
atggcagataagaatctccgttttctggtcgtcgacgacttttccaccatgcgtcgtatcatcagaaatttgcttaaagagctcggcttcaacaacgtcgaagaggcggaagatggcgcggacgcgctaaataagctgcgagccagcagctttgatttcgtggtctccgactggaacatgcctaacgttgacggcctggagctgctgcagaccattcgtgccgatgccgcgctggcggcgatgccggtattgatggtgacggcggaagcgaaaaaagaaaatattattgccgccgcgcaggcaggcgccagcggttatgtggtgaaaccttttacggcggcgacgctggaagaaaagctcaacaagatttttgaaaaattgggcatgtaa

SEQ ID NO: 178 - DNA, 1152 bp, Klebsiella sp.
  source 1..1152: mol_type = genomic DNA
ttggcggaagacagcgatctggaaaaaagcgaggcccccaccgcccaccggttggagaaggcgcgtgaagagggccagatcccgcgctcgcgcgagctgacctcggtactcatgctggtggccgggctcgccattatcctgatgtccggcagcaatatcactcgccaactggcggaaatgctgacgcagggcctgcactttgaccacgggatggtcagcaatgacaaacaaatgctgcgccagctgggaatgctgctgcgtcaggcggtgctggcattgctgccggtaatggccgggctggtgctggtggcgctggccgcgccgatgctgcttggcggcattttattcagcaccaagtcgctcaaattcgatcttaaacggctgaatccgctttccggtctgaaacgcatgttctccacccaggtgctggcggagctgttaaaagggatcctcaaagccacgctggtcggttgggtaaccgggctgtacctatggcacaactgggcggcgatgctgcatctgatgacccaacagccgctcgacgcgttggccaatgcgctgcagatgatcctctattgcggatttctggtggtgctcggattgacgccgatggtggcgtttgacgttttttatcagctgtggagccacttcaaaaagctgaagatgaccaaacaggatatccgcgacgaattcaaagatcaggaaggcgacccgcatgttaaagggcggatccgtcaacagcagcgggcgattgcccagcggcggatgatggccgatgtgccgaaagcggacgtgatagtcactaacccgacccactatgccgtggcgttgcagtacaacgataaaaaaatgagcgcgccgaaggtgttggctaaaggggcgggggagattgcgctgcgcattcgcgaactgggagcgcaacaccgtattccgatgcttgaagcgccgccgctggcccgcgcgctctatcgccatagcgagattggccagcatattcccgccaccctctatgccgcggtcgccgaagtgctggcctgggtttatcagctgcgccgctggcggcgcgagggcggcctgatcccgaaaaaacctcaacgtttaccggtgccggaagcactggattttgcaaaagagagtgactctgatggctaa

SEQ ID NO: 179 - DNA, 2079 bp, Klebsiella sp.
  source 1..2079: mol_type = genomic DNA
atggctaatttggcctccctgctgcgtttgccggggaatgttaaagatacgcaatggcaggttctggccggcccgatcctgatcctgctgattttgtcgatgatggtgctgccgctgccggcgtttatcctcgacctgctttttactttcaatatcgcgttgtcgatcatggtgctgctggtggcgatgtttacccagcgcacgctggacttcgccgcgtttccgaccattctgctgttctcgacgctgctgcgcctgtcgctcaacgtcgcctcgacgcgcatcattttgatggaagggcacaccggggccgccgccgcggggcgggtcgtggaggccttcggtcacttcctggtcggcggtaatttcgccatcggtatcgtggtgtttatcatcctcgtgctgatcaacttcatggttatcaccaaaggcgccgggcgtatcgccgaagtgggggcgcgtttcgtactcgacggcatgccgggtaagcaaatggcgatcgatgccgacatgaacgccgggcttatcggtgaagaagaggcgaaaaaacgccgcgcggaagtcacgcaggaagccgatttctacggctcgatggacggcgccagcaagtttgttcgcggcgatgccatcgccgggctaatgattatggtgattaacgtggtcggcggactgctggtcggcgtggtgcagcacggtatggagctgggggcggcggcggaaagctacaccttgttgaccatcggcgacgggctggtggcgcaaatcccggcgctggtgatctctaccgccgccggggtcatcgtgacccgggtggcgaccgatcaggatgtcggcgaacagatggtcggccagctattcaataacccaaaggtcatgctgctgtgtgccgcggtacttgggctgctggggctggtgccggggatgccgaacctggtcttcctgctgtttaccgccgcgctgctcgggctggcctggtggctgcgcgggcgcgaaaaacaggcgccgaaagccgccgacgaacccatcgtgcaggagaatacccaggccgcggaagccagttgggccgatgtccagttggaagatccgctgggcatggaagtgggttaccggctgattccgatggtcgattttcagcaaaatggcgagctgttggggcgtatccgcggtatccgcaagaaattcgcccaggagatgggctatctgccgccggtggtgcatatccgcgataacctcgaactggcgcccgcccgctaccgcattctgatgaaaggcgtggaaatcggcagcggcgaagcgcagcctgggcgctggctggcgatcaaccccggcaacgctatcggcgagctggcgggggataaaactatcgatccggccttcggtctggaggcggtgtggattgatagcgcgttgcgcgagcaggcgcaaattcagggctatacggtggtggaggccagtacggtggtggcgacccatctcaaccacctcatcggccagttcgccagcgaactgtttggccgccaggagacgcagcagctactcgaccgtgtgtcgcaggagatgccgaagctgaccgaagatttcgtaccgggcgtggtatcgctgaccacgctgcataaggtgttgcagaatctgctggccgagcgggtatcgatccgcgatatgcgtaccattatcgagacgctggcggaacatgcgccgacgcaaagcgatccttacgagctgaccaccgtcgtgcgcgtggcgctggggcgggcgattgcccagcagtggttccctggcaatggtgaaattcaggttatcggcctggatacccaactggaacgactgctgctgcaggcgctgcagggcggcggcggtctcgaaccggggcttgccgaccgcctgctggagcaggcgcggcaggcgctgcagcgccaggagatgctcagcgcgccgccggtgctgctggttaaccacgcgctgcgcccgctactggcgcgcttcctgcgccgtagcctgccgcagatggcggtgctttccaacctggagatcaacgacgatcgccagattcgcatgacctcaaccattggagcggcctga

SEQ ID NO: 180 - DNA, 435 bp, Klebsiella sp.
  source 1..435: mol_type = genomic DNA
atggacagattacgcactctgctggataaactggcggaaaccctgcgggcgctggatgacgtgctggcgcaagagcaacaattgctttgtgcggatgaactgcccggcgtggcgctgcagcgcgtcaccgatagcaagagtcaactgctggcgacggtggcctggcttgaacaacagcgcccggcgctggaagccaaccttgcgctacgcgcgccttaccccggccatgaaagcttcagcgaacgctggcggcagatccagcagttaagccagagcctgcgtgaaaagaatcagcataacggcctgttgctcaatcaacagatcgcccataaccagcaggcgctggcaattcttagcaagaataataaatcgctgtacgggccggacggtcagtcgcgcgccgccagtctgctgggccgtaaaatcggcgtctga

SEQ ID NO: 181 - DNA, 324 bp, Klebsiella sp.
  source 1..324: mol_type = genomic DNA
atgagtatcgatcgcacccagcggttacagccggtttccacggtgcagccgcgtgaaaccccggccgacaacccgcttcagccgcgcaaggccaccgcggcggaaaccgccgttagcgccaccaaagtgaaactgagcgatgcgcaggcgcggctgatgcagcccggcacccaggatattgatatgaaccgggtcgaggctatcaaacaggcgattcgtagcggcgaactgaaaatggacgcgggcaaaatcgccgacgccctgctgcaggatgcgcacaacgatatccagcgccttgccggtagcaaaacagataaggcctga

SEQ ID NO: 182 - DNA, 414 bp, Klebsiella sp.
  source 1..414: mol_type = genomic DNA
atgctcgacaaactggacgctgctctgcgttttggccaggaggcgctgaacctgcgcgcccagcgccaggaaatactggccgccaatatcgccaacgcagacacgccgggctatcaggcgcgggatatcgatttcgccagccaactgaataaagtcctgcaacaggggcgggtaaacggcaacggcatggcgctgaacctgacggcggcgcgtcatattccggcgcagaccatgcagccgccggagctggatctgctgttccgggtgccggaccagccgtcgatggacggcaacacggtggacatggatcgtgaacgcaccaacttcgctgacaacagcctgaaatatcagaccgacctgacgctgctcaacggtcagatcaaagggatgatgtcggtgctgcaacaaggataa

SEQ ID NO: 183 - DNA, 405 bp, Klebsiella sp.
  source 1..405: mol_type = genomic DNA
atgtctttactcaatatttttgatatctccggctcggcgctctcggcccagtcacagcggatgaatgtcagcgccagcaatatggccaacgccgatagcgtcaccggcccggatggcgaaccctatcgcgcgaagcaggtggtgttcgaagtggccgcggcaccagggcagcagacaggcggcgtgcgcgtctcgcaggtggtcgatgacccggcgcccgcgcggatggtttatcaaccgggtaatccgctggccgatgctaagggttatgtacggatgccgaacgtcgatgtggtaagcgaaatggttaacaccatctccgcctcccgcagctatcaggccaacgtcgaagtgctcaacaccactaagtcgatgatgatgaaaaccctgacgttgggtcagtaa

SEQ ID NO: 184 - DNA, 678 bp, Klebsiella sp.
  source 1..678: mol_type = genomic DNA
atggctattgccgcaaccaccaatgaatcgaccaacaacgcggttcttgattccgccagcaaaagcagcaccagccaggatctgcataacagcttcctgacgttgctggtggcgcagttgaaaaatcaggacccgaccaacccgatgcagaacaacgagctgacctcgcagctggcgcagattaataccgtacagggcatcgaaaaactcaacaccacccttggcgcgatctccgggcagatcaacagcaaccagtcgctgcaggccagcgcgctgatcggccacggcgtgatggtaccgggtaataacattctggtcggcagcaaagaaggccaggtcagcaccacgccgtttggcgtcgaactggagcgcgccgccgatcaggtcacggcaaccatcaccaacgccaacggccaggtggtgcggaccattgagattggcggcctcaccgccggggtgcacgccttcacctgggacggctcgctggatgatggtaccgccgcgccggacggcgcctataaagtggcgattaacgccaagggcaacggcgagcagctggtggcgcgtagcctgcatttcggcatggttaacggtgtgattaacgacagcaacggcgcgaagctggatctcggcctggccggtaacgccagcctcgaagaagtgcggcagatcctgtaa

SEQ ID NO: 185 - DNA, 1215 bp, Klebsiella sp.
  source 1..1215: mol_type = genomic DNA
atggccttttctcaggcagtcagcggcttaaatgcggcggccaccaatctcgacgtgattggcaacaatatcgctaactccgcgaccgcgggttttaaatccggcagcgtgtcgttcgccgatatgttcgccggttcgcaggtcgggctgggggttaaagtttccgggatcacccagaactttaaaggcggcaccactaccggcaccagccgcgcgctggatgtggcgattaacggcaacggcttcttccgcatgcaggataaagacggcggtattttctatacccgcaacggccagtttaagctggacgaaaaccgcaatctgaccaatatgcaggggctgcagctgaccggctatccggccgccggcacgccgccgaccattcaacagggcgccaacccggtaccgctgagcattccggaaggaatgatgaacgccaaagcgtcgacttccggcgaaatggtcgccaatctgaagtcgacgcataaagtgccggagaacaagaccttcgacccggcgaagcaggacagctataactacgtcaacaccatcaccgcctacgattcgctgggtaacgcccacaacatcaacgcctatttcgtgaaaaccgacgataacaaatggcaggtctatacccaggacggcagcgcggctgcggtcagcgcgggcaccatggagttcaataccagcggcaacctggtcagcgttaacggccaggcgggcgagttcagcatgaccattccgatgagcgcgaaagacggcgccccggcgcagaacttcacgctcagcttcgccggtagtatgcagcagaacgtcggtagcgattcggtgagtaaagtggcgcaggatggttatgccgccggtgaatacaccaacttccagattaacgatgacggtaccgtcgtcggtatctattccaaccaacagacccaggtcctggggcagattgtgatggccaacttctccaacccggaagggctggcgtcgcagggcgataacgtctggcaggagaccggcgcctccggccagccacgggtgggactctccggcggcggcggttttggcaagctgaccagcggcgcgctggagtcttccaacgtcgacctgagccaggagctggtgaacatgattgtcgcccagcgtaactatcagtccaacgcccagaccatcaagacgcaggattcgatcctgcagacgctggtcagcctgcgctaa

SEQ ID NO: 186 - DNA, 756 bp, Klebsiella sp.
  source 1..756: mol_type = genomic DNA
atggatcacgcgatttataccgcgatgggcgccgcgcgccagacgctggagcgacagtcgattaccgccaacaatctcgccaacgcctcgacgccgggctttcgcgcccagctcgccgcgctgcgcgcggtgccggttgacgggccgagcctcgccacccgcacgctggtggcggcctcgacgccgggcgccgatatgagccagggggcgctgaactacaccgggcgcccgctggacgtggcgctgcagcaggacggttttctggcgctcagcctgccgggcggcggcgaagcctatacccgcaacggcagcattcaggtttcggctaacggtcagttgacggtgcagggaatgccgctgcagggcgacggcgggccgattgaggtaccgccatcggcggaaatcactatcgcggccgacggcaccatttcggcgctgaatcccggcgacccgccgaacaccatcgcgcagattggccgcctgaagctggtgaaagctgatgcgcgcgaagtgatgcgcggcgatgacggcctgttccgcctgaccccggaaacccagcagcgccgcggcaatctgttggccaacgacccgcaggtgcgggtcatgccgggcgtgctggagggcagtaacgtcaagccgatggagaccatggtcgatatgatcgccaacgcccgtcgctttgaaatgcaaatgaaggtcatccacagcgtggatgagaacgaacagcgggcgaactctctgctgtcagtaagctga

SEQ ID NO: 187 - DNA, 783 bp, Klebsiella sp.
  source 1..783: mol_type = genomic DNA
atgattccctctttatggattgcgaaaaccggtcttgatgcgcagcagaccaatatggacgtgatcgccaacaacctggcgaacgtcagcaccaacggttttaaacgccagcgcgcggtgtttgaagatctgctgtatcagaccatgcgccagccgggggcgcagtcctccgagcagaccacgctgccttccggcctgcagatcggtaccggcgtgcgcccggtcgccaccgagcgtctgcatagccagggcaacctgtcgcagaccaacaacagcaaagacgtggcgattaaaggccagggcttcttccaggtgctgctgccggacggcagccaggcttacacccgcgacggctcgttccagatcgatcagaacggccaactggtgaccgccagcggcttccaggtgcagccggcgatcaccataccggccaacgcgttgaccattaccatcgcccgcgatggcatcgtgagcgtcacccagcagggtcagaccgccgcccagcaggtcgggcaactgacgttaaccacctttattaacgacagcggcctcgaaagcgtgggcgagaacctgtaccaggaaactgaaagctccggcgcgccgaatgagagcacgccgggcctgaacggcgccggtatgctctaccagggttatgtcgagacctctaacgtcaacgtggcggaagagctcgttaatatgatccagacccagcgcgcctatgagatcaacagcaaagcggtttcgacgtcggatcagatgctgcagaaactgacccagctgtaa

SEQ ID NO: 188 - DNA, 1650 bp, Klebsiella sp.
  source 1..1650: mol_type = genomic DNA
atgtccaatagcttaatcaatacggcgatgagcggactgaatgcggcgcaggtggcgctcagtaccgtcagcaacaatatctctaactacaacgtggcgggttataaccggcagacggcgattctggcccaaaacggcggcctggccaccatgaacggttttatcggtaacggcgtgaccgtgaccagcgtgaaccgcgaatataaccagttcatcaccaaccagttgcgcggcgcgcagagcgcggccggttcgcagaacgcctactacgaaaagatttcgcagatcgataatctgctggccagcaaaaccaataccctgtcggggagcctgcaggattttttcaccaatttgcagaacctggtgagcaacgccggcgacgacgcggcgcgccagacggtgctgggcaaggctaacggcctggttaaccagtttaataataccgataaatatctgcgcgacatggatagcggcgttaatcagcaaattaacgacaccgcccagcagattaacagctatagccagcagattgcccgtcttaacgatgagattacccgcctgcgcggcagcgccagcggcgaaccgaacgcgctgctcgatcagcgcgatcagctggtcagcgaactcaaccaactggtgggcgttcaggtaacccagcaggacggcgacgcctataccgtctccttcgccaacggcctgaccctggtgcagggcaacaacagctatcaggtggaagcgatcccttccagcagcgacccttcgcgcctgactctcggctataaccgcggcagcggcgccaacgaagtgccggaagggcagatcaccagcggcagcctgagcggcgtactgcgttttcgcagcgagacgctggacggcgtgcgcaatcagctcggccaactggcgctggcgatggccgatagctttaaccagcaacaccgcgagggcttcgatctcaacggtgagcaggggggcgatttcttcagcttcagcggcgcgcgggtggttgataacacgcgcaataccggcgatgcctcgctgtcggtgtcgtataccgataccagcaaggtaaaagccaacgattaccgcgtagagtatgacggcgccaactggcaggtcagccgtctgccggacaacgtcaaagtcaacgccaccgccggtaaagatgcagccggtaacccgacgctgagctttgatggtctcgaagtcagtattgatggcaacccgcagcagaaagatagctttacggtgaagccggtcagcgacgtggcgggcaacctgaaagtggcgattaccgattcagcgaaaatcgccgccgccggtaaagatgacagcggccgcggcgataacgataacgctaaaaaactgctcgatctgcagaccaaaaacctggtggatggcaaagcgacgcttagcggcgcctacgccgggatcgtcagcggcgtgggcaaccagaccgccgccgcgaaggtgagcagcgaatcgcagtccagtatcgtcacccaactggcccgtcaacagcagtcgctctccggggtaaacctcgacgaagagtatggcgagctcctgcgtttccagcagtactacatggcgaacgcgcaggtgatccagaccgccagctcgctgttcgacgcgctgctgaatattcgttga

SEQ ID NO: 189 - DNA, 783 bp, Klebsiella sp.
  source 1..783: mol_type = genomic DNA
gtgatcccctttgacagcgcccagcttagcgaatggctcagccagtatttctggccgctgctgcgtattctggccctcatcagcaccgcgccggtgctgagtgaaaagcagataagcaaaaaggtgaaaatcggtctcggcggcctgattgcgatactgatcgccccaacgctgcccatcaacgcgatccccattatctctgctgccggtttatggctggcgattcagcagatcctgatcggcgcggcgctggggctaacgatgcagctggctttcgccgcggtacgcctcgccggcgaagtcatcggcatgcagatggggctatcgttcgccaccttcttcgaccccagcggcggccccaatatgccggtgctggcgcggctgcttaacctgctggcaatgctgctgtttttaagctttgacggccacctgtggctgatctcgctgctggccgacagtttccataccctgccgatccagagccagccgctgaacggcaacggtttcctcgcgctcgcccaactgggttccttaatctttatcaacggcatgatgctggcgttaccgctgatttgtctgttgctcacgctcaatatcgccctgggtctgctcaaccgtatgacgccgcagctatcggtattcgtgatcggtttcccggtgaccatgacctttggcattatgaccctcggcatgatgatgccgatgctggcccccttctgcgaacacctgtttggcgagatgttcgatcgcctggcggccgttatcggcggtatgacgttttaa

SEQ ID NO: 190 - DNA, 270 bp, Klebsiella sp.
  source 1..270: mol_type = genomic DNA
atgacgccggaatccgtgatggccattggcaccgaagcgatgaaggtcgccctggcgctggccgccccgctgctgctggcggcgctaatcagcgggctggtggtcagcctgctgcaggccgccacccagattaacgagatgaccctatcgttcatccccaaaattctcgcggtagtggcgacgattattatcgccggaccgtggatgctgaacctgctgctggactatatgcgcacattattcagcaacctgcctaatattattggctag

SEQ ID NO: 191 - DNA, 762 bp, Klebsiella sp.
  source 1..762: mol_type = genomic DNA
atgttccctactctgttcgcttgccttcgccgtcgggcgccgtggctcgccgccgcgttactgctgctgagccctgccgccttcgcacagctgccggggcttatcagtcagccgctggccaacggcggccagagctggtcgctgccggtacagacgctggtgctgttgacctcgctgaccttcctgccggcgatgctgctgatgatgaccagttttacccgcatcatcatcgtactgggtctgctgcgtaacgcgctgggcacgccttccgcgccgccgaaccaggtgatgctcgggctggcgctgttcctgaccttctttatcatgtcgccggtgttcgacaaggtctatcaggaggcgtacctgcccttcagccaggacaagatcggccttgatacggcgctggataaaggcgcgcagccgctgcgcgaatttatgctgcgccagacccgcgaaagcgatctggcgctgtacgctcgtttggccaaccagccgccgctggccgggccggaggcggtaccgatgcgtattctactgcccgcctacgtcaccagcgaactgaaaacctccttccagattggttttacggtgtttattccgttcctgattatcgatttagtggtcgccagcgtgctgatggcgctggggatgatgatggtgccgccggcgacgatctcgctgccgtttaagctaatgctttttgtgctggtggatggctggcagctgctgctgggctcgctggcgcagagcttctattcctga

SEQ ID NO: 192 - DNA, 417 bp, Klebsiella sp.
  source 1..417: mol_type = genomic DNA
atgagtgatcctaagcaaccgtctggcgcaggaaaggaatccgtagacgatctgtgggctgatgcgcttaatgaacaacagtcgtctgagaaatccagcgccaccaccgatggggtgttcaaatcgctggaggcccaggatgcgctcggcagcctgcaggatatcgatctgatcctcgatattccggtgaagctgaccgttgaactggggcgcaccaaaatgacgattaaagagctgctgcgcctgtcgcagggatcggttgtcgcgctggatggcctggcgggcgaaccgctggatatcctgatcaacggctatctgatagcccagggcgaagtggtggtggtggccgataaattcggtgtacgcattaccgatatcattaccccgtccgaacgtatgcgtcggttgagccgctaa

SEQ ID NO: 193 - DNA, 1008 bp, Klebsiella sp.
  source 1..1008: mol_type = genomic DNA
atgggcgatagcattctttcacaggcagagatcgacgccttgctcaacggcgacaacacgggcgacgagcccgagacggttgtcggcggtaaagagagcgaggttaagccttacgatccgaatacccagcgccgcgtggtgcgtgaacgcctgcaggcgctggaaattatcaacgagcgttttgcccggcagttccgcatgggattgttcaacctgttgcgccgcagcccggacatcaccgtcgggccgattaaaattcagccgtatcacgagtttgcccgcaacctgccggtgccgaccaaccttaatctggtacacctgaacccgctgcgcggcacggcgctgtttgtcttcgcgccgagcctggtgtttatcgccgtcgataacctgttcggcggcgacgggcgtttcccgaccaaggtcgaaggccgagaattcacgcccaccgaacagcgggtcatcaaacgcatgctgcgcctggcgctggacgcctacggcgacgcatggagcgcaatctataagattgacgtcgaatacgtgcgtgcggaaatgcaggtcaaattcaccaacatcaccacctcgccgaacgatatcgtggtgaccacgccgttccaggtggagattggcgcgctaagcggtgaattcaatatctgtattcctttcgccatgattgagccgctgcgtgaactgctgaccaatccgccgctggagaattcccgccaggaagatagccagtggcgcgaaacgttggtcaagcaggtgcagcactctgagctggagctgattgccaacttcgtcgatatcccgatgcgcttatcgaaaatacttaagctgcagccaggcgacgttttaccgatagataaaccggatcgcattatcgcccatgtcgacggcgtgccggtgctgaccagccaatatggcaccttaaacgggcagtacgcccttcgtgttgaacacttgattaaccctattttgaatgctctgagtgaggaacagcccaatgagtga

SEQ ID NO: 194 - DNA, 1179 bp, Klebsiella sp.
  source 1..1179: mol_type = genomic DNA
atgaatctcaatgctttgcccgcccttagcctacccggcgacgccagcgccctggcggatctggcgctcgatgacgctcagctgtcatctgcctttgcgcaactgcttggcgcgcgcttcacgcccgcgcaaagcggcacgctgccgccggcggcgctgagcgccgacgatgaaagcgccccgccgctaagccgtaaccagcttaaccagctgctggccgcgcttggcgaacgcggcacgctgctgggcaatgcgctaccggcatcggcggaaaacgccgtcgacacaacagcgaaaaaagaggatacgcgcccggatacgccaccgctcagcgaggcgaaaactttagatccggcgacgctgcaggcactgtacgccatgctgccggcggcaatcgtcgccccgccgccgcagaccgcgctaccgcaggctcaaccagctgcgaccagcggcgataaaaccgcgctccacatcggcaacgcggcgactcagcatgtcgcgaacctgccggaagccgaaccggcggcgaagccgcagccgggcacggatccccttagcgggcaaaatctgccttctcctgttgtgcagaccgcgaccacgccgcagcacgatatggccgaacctccggcggagaatactccggcgcagccagccgccgtcgctttcgccgccgccagccccagccagagcgccacgcccgccagtgcgctggtaaccgcgccgccgacgccgcagcttaatgcccagctcggcagcccggaatggcagcaggcgctgagccagcaggtgctgatgttccaccgcaacggccagcagagcgccgaactgcgcctgcatccgcaagagctgggggcgctgcagatcaccctgcagctcgacgataaacaggcgcagctgcacatcacttccgcacacggtcaggtacgcgcggcggtcgaggcggctatgccgcagctacgtcacgcgctggctgagagcggaatcaacctcggtcagagcagcgttggcggcgaggcgacgccgcagtggcagcagcaaaacggtaacggtgaaggccgcgccagctacgctgaacgccatggcggcggcagcgatgccgcgccgattgagccagtcagcgccccggcagccctgcagcgaatggccaaccagctcaacggcgtcgatattttcgcctga

SEQ ID NO: 195 - DNA, 447 bp, Klebsiella sp.
  source 1..447: mol_type = genomic DNA
atgaaagcacagtcccctttgattaccctgcgcaatctggcccaggaggccgtcgaacaggcggcgcagcgtttgggccaggcgcgccaggcgcaacaggccgccgagcaacagctgacgatgctgctgaactatcaggacgagtaccggcaaaagctcaacagcaccctttctggcggcatggaaagctcgcgctggcagaactaccagcagttcattgccaccctcgaacaggctatcgaacaacagcgccagcagcttctgcagtgggggcaaaaggtggatcacgccgtgaagcagtggcaggaccatcagcagcggctgaacgcctacgataccctgcacacgcgcgcgcaaaacgccgagctgcagttggaaaacaaacgcaatcaaaaattgatggatgagttcgcgcaacgcagtacacaaaggaataccgcttcatga

SEQ ID NO: 196 - DNA, 1362 bp, Klebsiella sp.
  source 1..1362: mol_type = genomic DNA
atgaccgcccgtctcggccgctggctgaattcgcttgaagcgctggagcagcgtatcgcccgaacgccgaccgtacgacgctatggtcgtctgacccgcgccaccggtctggtgctggaggccaccggcctgcagctgccgctcggcgcaacctgccttatcgagcgcgacggcgggggcggcgtgcaggaagtggaaagcgaagtggtcggctttaacgatcagcggctgtatctgatgccgctggaagaggtcgaaggaatcgtgccaggggcgagagtttacgcccgcagcgcggcggacggccagcacgccggtaaacaactgccgttgggcccggcgctgctgggacgcgtgctcgacggcggcgccaggccgcttgatggcctgcccgcgccggaaaccggctatcgcgcgccgctgattaccgcgccgtttaacccgctgcagcgtacccctatcgagcaggtgctggacgtcggcgtacgcaccattaacggcctgctgaccgttgggcgcggccaacgtatggggctgttcgccggctccggcgtcggtaaaagcgtgctgctgggcatgatggcgcgctacacccaggccgacgttatcgtcgtcggcctgatcggcgaacgcggccgggaagtcaaagactttatcgagaatattctcggcgccgaaggccgcgcgcgttcagtggtgatcgccgccccggcggacgtatcgccgctgctgcgcatgcagggcgccgcttacgccacccgcatcgccgaagattttcgcgatcgcggccagcacgtgctgctgatcatggattcgctcacccgctatgcgatggcgcagcgagaaatcgcgctggcgatcggcgaaccgcctgcaaccaaaggctatccgccatcggtattcgccaaactgccggcgctggttgaacgcgcgggcaacggtatcagcggcggcggttcgatcaccgccttctataccgtactcaccgaaggggacgatcagcaggatccgatcgccgattcggcgcgcgcgatcctcgatggtcacgtggtgctgtcccgacgtctggcggaggccggtcactacccggccatcgatattgaagcctcgatcagccgcgcgatgacgtcacttatcgatgacgaacattatcgccgggtacgcacctttaaacagatgctggccagcttccagcgcaaccgcgatttaataagcgtcggcgcctatgccgccggtagcgacccgctgctggataaagccattaccctgtatccgcaaatggagacctatctgcagcagggaattttcgagcgctgcggttatgacgaagcctgtctgcagctacagcagctgattgtttaa

SEQ ID NO: 197 - DNA, 702 bp, Klebsiella sp.
  source 1..702: mol_type = genomic DNA
atgtctgatcgcattaattccatgccctggcagccctggtcactcaacgacctcggcgacgcggcgccagcttttgaaccgccgctaccgacgttggataacgaagccttgcagcctgacgctcaggcggaactgcagcagcagcttgccgccctgcgcctgcaggccgagcagcaaggtcagcaatccggttatgccgatggccagcaaaaaggctatgaagccggatttcagcagggacttgaggagggccgccagcaggggctgctggaagcacagcagcagcaacagccgctgaccgaccactggcagcagctggtgagcgagtttcagcacacccttgatgcgctcgattcggtcatcgcctcacgcctgatgcagctggcgctaaccgccgcgaaacaggtgcttggccagccgccggtctgcgacggcactgccctgctgacgcaaattcaacagttgatccagcaagaaccgatgttcaacggcaagccgcagctgcgcgtacacccgcaagattatgagcgcgttgagcagcagttaggcaccaccctcagcctgcacggctggcggttgctggcggatggcgatctgcatcccggcggctgtaaggtcagcgccgaggagggcgatctcgacgccagtctcgccacccgctggcacgaattgtgtcgcctcgccgcgccgggagatctgtga

SEQ ID NO: 198 - DNA, 993 bp, Klebsiella sp.
  source 1..993: mol_type = genomic DNA
atgagcctgaccggaaccgataaaagcgccattttactgatgacgctgggcgaagaccgcgcggcggaggtgctcaagcacctctcctcccgcgaagtccagcttttgagcggcaccatggccggcatgagccaggtctcgcataaacagctcggcgagatcctgtctgaatttgaagacgatgccgaacagttcgccgccctgagcgtcaacgccagcgactatttgcgttcggtgctggtgaaagcgctgggcgaagagcgtgccgccagcctgctggaggatattctcgaatcccgcgaaaccacctccgggatggaaacgctcaactttatggagccgcagagcgccgccgacctgatccgcgatgaacacccgcagatcatcgccaccatcctggtacatctcaagcgcggccaggcggcggatattctggcgcagttcgatgagcgcctgcgcaacgacgtaatgctgcgtatcgccaccttcggcggcgtgcagccggcggcgctggcggagttgaccgaagtactcaacaacctgctcgacggccagaacctcaagcgcagcaaaatgggcggagtgcgtaccgccgccgagattatcaacctgatgaaaacccagcaggaagaagccgttatcgacgcgatgcgcgaatacgatggcgagctggcgcagaagatcatcgacgagatgttcctgttcgaaaacctggtggaggtcgacgatcgcagtatccagcgcctgctgcaggacgtggacagcgaatcgctgctgattgctctgaagggcgccgagcagccgctgcgcgagaagttcctcaaaaacatgtcccagcgcgcggccgatattctgcgcgacgatctcgccaaccgtgggccggtgcgcatgtcgcaggtggaaaacgaacagaaagcgattctgctgatagtcagacggctggcggagagcggcgaaatgattattggcggcggcgaggacacctatgtctga

SEQ ID NO: 199 - DNA, 1677 bp, Klebsiella sp.
  source 1..1677: mol_type = genomic DNA
atgagtgcctcgttatccgccggcgaaagccgcgacaacggcctgcaggccatctggagccgtctgcgcgccaatccaaaaataccgctgctggtggccgcctccgccgctatcgccattatcgtcgcgctgctgctgtgggcgaaaagcccggattatcgcgtgctgtacagcaatctgaacgatcgcgacggcggcgctatcgtcacccaactgacgcagatgaacatcccctaccgcttcgccgagaatggcgcggcgctgatgatcccggcggagaaagtgcatgaaacccgtctgcgtcttgcgcagcaggggctgccgaagggcggcgccgttggcttcgaactgctggatcaggaaaaattcggcatcagccagttcagcgaacagattaactatcagcgcgctcttgaaggcgaactctcacgcactatcgagtcgctggggccggtgcagaatgcgcgggtgcacctggcgctgccgaagccctcgctgttcgtccgcgagcaaaagtacccttccgcttctgtcaccctgacgctgcagccgggccgtgcgctggatgatggccaaatcaacgctatcgtctatatggtatcgagtagcgtcgccggcctgccggctggcaacgtcaccgtggtcgatcaggccggacgcctgctgacgcagtctgacggcaccgggcgcgacctcaacgcctcgcagctgaaatatgccagcgaagtggaaagcggctatcagcgacgcattgaaacgatcctggcgccggtggtcggcagcgccaacgtccgcgctcaggtgaccgcacagatcgatttcgcctcccgcgagcagaccgacgaacagtaccagccgaaccagcagcccgataaagcggcgatccgttcgcagcaaaccagcagcaacgagcaaatcggcggaccgcaggtgggcggcgttcctggggcgttatccaaccagccgagcgcgccggccaccgcgccgattgaaaccgctaaaccggcggccaataacgccagcaacgcgaccaacacccgtagcgccgcggccaacagcggcccgttaagcacccgacgcgacgccaccaccaactacgaactcgatcgcaccatccgccatacccaacaaaaagccgggacggtggagcgtctgtcggtcgccgtggtggtgaactacagcctcaacgccgacggcaaagcgcagccgatgagtaaagaacaactggcgcagattgaagctctggtgcgcgaagcgatgggctactccagcagccgcggcgatagcctacaggtggtcaatacgccatttaccgacaatcagatgaccggcggcgagctgccgttctggcagagccaggcgtttatcgatttgctgctcgaactgggacgttatctgctggtgctgctggtcggctgggtattgtggcgcaagctgattaagccacagctgcagcagcgtcaggccgcacagcaagccgcggtcgccgccgccagcgcgccggtcgctaaagcgaacgacagccataaaccaagcaatgaggagctggcgctgcgccgtaaatcgcagcagcgcgtcagcgcagaagtccagagccagcgcatccgcgaactggcggataaagatccgcgggtcgtcgctctggtaatccgccaatggatgagtaacgaactatga

SEQ ID NO: 200 - DNA, 315 bp, Klebsiella sp.
  source 1..315: mol_type = genomic DNA
atggcgattcagggtattgaaggcgtactgcaacagatgcaggcgatggcgattcaggcgggaaataacgggcagaatagcgtaccgcagggagtcagttttgccagtgaattgaccgccgcgctgggtaaaatcagcgatacccagcaggccgcgcgtcaacaggcgcaaaacttcgagctgggcgtgccgggtatcagcctgaatgatgtgatggtcgatttacagaaatcttcagtttcgttgcagatgggcgtgcaggtgcgcaataagctggtggcggcctatcaggacattatgaatatgccggtttag

SEQ ID NO: 201 - DNA, 408 bp, Klebsiella sp.
  source 1..408: mol_type = genomic DNA
atggaacgccaacagcagcttttagccgcctatcagcagatccataccctgagcagccagatgattgccctggcgcgcgccgggcaatgggaagcgctggtggaaatgcagtttgcctacgtgacggcggtcgagaaaaccgccgaatttaccggcaaagcggggccatcgctggctctgcaagagatgctcaacgctaagttgaagcagattatcgacaatgagggagtactgaaagggctattgcaacagcggatggaacaattaaaaacgctgatcgatcaatccacccgccagaacgcggtcaacaacgcttacggccagttcgacgatcgttcgctcctgcttggcgaactgcagtccgataacgtggtcgccattaagagcaaaagcgaagaattacaataa

SEQ ID NO: 202 - DNA, 411 bp, Klebsiella sp.
  source 1..411: mol_type = genomic DNA
atgtataaccgtagcggcacgcaagcctacgcacaggtcagtctggaaagcagcgcgatgagcgccagtccgcaccagttaatcgtaatgttgttcgacggggcgcttaacgccctgcttcgcgcacgcatcctgatgaatcagggggatatcgccggtaaaggcctggcgctgtcgaaggcaatcaacatcatcgacaacgggttgaaaagcggcctcgaccaccagcaaggcggcgaaatcgccgataatctggcggcgctgtacgattatatgaagcgacgcctaatgcaggccaacctgcataatgacgaagcggcgatcgtcgaagtggtcaagctgctggagaatatcgccgatgcctggcgacaaattgggccaaactatcaacctgctcagggtacattgtga

SEQ ID NO: 203 - DNA, 1404 bp, Klebsiella sp.
  source 1..1404: mol_type = genomic DNA
atggcatcgatcagttccttaggcatcggctcaggactcgacctcaacggtttactggacaaactcagcaaggcggaacaacagcgtctgacgccgtataccactcagcaaaccagctataacgcgcagctaacggcttacggcacgttaaaaagctcgctggaaaaattcgataacctgagcaaggagctggctaagccggagtttttcaacagcaccaccgccaccaagcacgaccagttcaccatcaccaccaccaataaggcggtgccgggcaactatagcgttgaagtgcagaagctggcgcagccgcaaaccctgaccacccaggcgacaattaccgaccaacaagagaaacttggaaccgccggcaacagcgaccgctctattacgatcaccgccggcgacccgccaaaagaaaccacgattccgctgagcgacgaccagacctcgctgctggagatgcgcgatgccatcaacggcgctaaagccggcgtcaccgccagcatcatgcgcgtcggcgacaatgactatcagctggcgctgagctcctccaccaccggcgaaaaaaacaccgtgtcggtgcaggtcaataacgacgataaactcggcgctatcctcaactacgacagcaatagcaaaagcggcgcgatgaagcagaccgttgccggacaggatgcggagattatcgtcaacggcaccaaaattaagcgcagcaccaactcgattgccgatgcgctgcagggcgtcaccatcgacctgaaaaccaccaccaaagctggcgaaccgcaaaacctggtgatcggcatcgataaaagcggcaccgccgacaaaatcaaagagtgggtggataattataattcgctgctcgacaccttcggctcgctgaccaaatacaccccggtgaaaagcggtgaagcgcaaagcgccaagaacggcgcgctgctgggcgataacaccctgcgcggcattcagtcatcgattaaaagcgcgctcagcgccgctcaggacaacccggagctgaaaggccttggcaacctcgggatctcaaccaatcccaaaaccggcaagcttgaagtcgacagcaccaagctcaataaagcgctcgacgagaaaccggatcaggtcgccaacttcttcgccggcaacggcaaagacaccgggatggccaccgaaatccataatgaaatccagaactatattaaagcgggcggcatcatcgagaactcgaccaaaagcatcaacaccaatctcgatcgcctgaatatccagatcgacactgtttccgacagcatccagaacactatcgatcgctataaacaacagtttgttcagttggataccatgatgtccaagctgagcagcacgggtaactacttacaacagcagttctcgtcccagtaa

SEQ ID NO: 204 - DNA, 1263 bp, Klebsiella sp.
  source 1..1263: mol_type = genomic DNA
atggcacaagtaattaataccaacagcctgtcgctgatggcgcaaaacaacctgaacaaatcccagtcttccctgggcacggcgattgagcgtctgtcttccggtctgcgcatcaacagcgccaaggacgatgccgcgggtcaggcgatctccaaccgttttaccgctaacatcaaaggcctgactcaggcttcccgtaacgctaacgacggtatctccctggcgcagaccaccgaaggcgcgctgaatgaagtcaacgataacctgcagaacatccgtcgtctgactgtgcaggcgcagaacggttctaactcttccagcgacctgcagtccatccaggatgaaatcacccagcgtctgtctgaaattgaccgaatttctcagcaaaccgatttcaacggtgtgaaagtactgagtagcaatcagaagctgactattcaggttggcgctaatgataatgaaacgattgacatcgacctgaaagatatcaataaaagcactctaaatttagataccttctctgtggcattcggcgtcaataagggggatgtcggtaaaactgctgttgctgcaaatgattccattcaactggataaaacagtaacaatcgctgccgggcaaaaagcaaattctaaagacgttactggttatgttaaagatgataagggtgatatttatgctcgtactggcgctacagagtattacaaagtagaaagcattgacaaagatggaacagcaacttttgcagcggcaccggcagcaccatctggtacgactaaagcagtaaccgaaggtaagatttctgttaaggcaaccgataatgcagataaagatttagctaatactttcgcgtacacgggtacagagaaaggtgctcctaaatatgtgattaaagatactagtggcgcgactgaccgttattacgcggctacttttgatgccgatggtaaagtggttcgcggcaacgagctcagttcaactcctgctactgaagatccgcttgaagcattagacaaagccctgtcccaggtagaccaactgcgctcctccctgggtgcggtacagaaccgtttcgattctgttatcaacaacctgaacagcaccgtgaacaacctgtccgcgtcccagtcccgtattcaggatgctgactacgcgaccgaagtgtccaacatgagccgcgcgaacatcctgcagcaggcgggtacctccgtgctggcgcaggctaaccagtcgactcagaacgtcctgtctctgctgcgttaa

SEQ ID NO: 205 - DNA, 4065 bp, Klebsiella sp.
  source 1..4065: mol_type = genomic DNA
atgcggcgactggtaattatcggcaacggtatggcggcaacccggctggcagagaagctggcggcgcgcgcgcaggggcgtttcatcatcacgctactcggcgatgaaccgcagccggcctacaaccgcattcagctctcgccggtactcggcggggaaaaaacggcgtcgcaaatccaactgctgccgacggagtggtacgcccagcatggtgtccgcgtgcggcttggcgaggcggtcagcgaagtggatgtcgcggggcggaggctgcgcgtcggcggcgactggctggaatgggatgaactggtgttcgccaccgggtcgcagccgtttatcccgtcgctgccgggtattgagcgcccgcaggtattcgcctttcgcaccctggcggatgttaaagacattctggcaatccccggcccggcggtggtaatcggcggtggcgtactgggcgtggaggccgcggcggcgctgcgccgtcacggtggggaagtgacgctactccatcgcggtagcggtttgatggagccgcttaccgatgcttttgccgcggaacagttaaaacaacagcttgaggcacgcggcatccgctgcgagctggagagccgaattgccgctattgacgagcagcatgtgctgctggaagacgggcgcgcttttgccgccagccgggtggtgttggccaccggcgtgcggccggatatccgtctggcgacgcgcagcggcgtagcctgccagcgggggattatcgtcgaccgccggatggcgacctcgctgccggggatcagcgctatcggcgagtgttgtgagattgacggccaaacctggggactggtggctccctgcttgcgccaggccgatgtattagttgaacgcctgtgcggggctcccggcgaagaattcagctggcaggacggcggcacccggctgaaggtcaccggcatcgacttttttagcgccggcgaacagcgggccggcgagcaggaccagatctacagcagttgggaccccatcgatcgccactaccgtcgcctgctgatccgcgatggcaggttgcacggcgtgctgctgctgggcgactgccagcaggcggcgccgcttaccgcgctgctcggcagcgataggtccccttcagtggagtggctttttgacccttctataacgcagccgcgggctgcaggaaaaatgacgatgactaaacctgtattagtgctggtcggacacggcatggtcggccaccattttcttgagcaatgcgtgagccggaacctgcaccagcagtaccgtatcgtggtttttggcgaagagcgctatgccgcctatgaccgggttcatctctcggaatatttcgccgggcgcagcgcagagtcgctatcgctggtagagggagacttttttagccagtacggcattgagctgcggccaggagagacgatcgcggaaatcgatcgtccggcgcgaatcgtgcgcgatgcccacggccatgaaacccattgggataagctggtgctggcgaccggctcctatcccttcgtgccgccaatccccggtaacgatgaagaaggctgcttcgtctatcgtacgcttgacgatcttgaccgcattgccgcccgcgccgccactgcgcagagcggggtggtcattggaggcgggctgttggggctggaagccgccaacgcgttaaaacagttggggcttaagacccatgtggtggagttcgccgccaacctgatggcggtgcagctggataacggcggcgcggcgatgctgcgggagaaaattctcgatcttgaggttggcgtgcacaccagcaaagcgacgacggcgatagtgcgcaccgccgacgggctgcagcttaacttcgccgatggcggcacgctgcagaccgatatggtggtcttttccgccgggatccgcccgcaggatgcgctggcgcgcggcgcaggcctggcaatcggcgaacgcggcggtatcggtatcgatgaccactgccggacctcggacgcagatatattggcgattggcgaatgcgcgctgtgggacaacaaaatttatggtctggtagcgccgggctaccacatggcgcgcaccgcggcggcgcagttggctggtgaagcggcccgctttagcggcgccgatatgagcaccaaactcaaactgctcggcgtggatgtagcgtcgtttggcgatgctcacggacgtacagctggttgccagagctatcaatggaccgatggtccgcggcagatctacaaaaagatcgttgtcagcgctgatggcaaaacgctgctgggcggcgtattggtgggcgatgccggtgactacgcgacgctgttgcagatgatgctgaacggcatggcgctaccgtctcgtccggaaagtctgatcctcccggccatggatggggccacgacgaaagcgctgggggtggcggcgctgccggatagcgcgcagatttgctcttgccataacgtcagtaagggagacatttgccaggcggtcagcgccggagcgggcgatatggcggcgataaaaagctgcactaaagcggcgaccggctgcggaggctgtagcgcgctggtcaaacaggtgatggaacaccagcttaccgggcagggcgtcgaggttaaaaaagatatctgcgagcattttccgtggtcgcgacaggagatctatcatctggtacgagtcaaccacatccataccttcgatcagctcattagccgttacgggcagggccacggctgtgaagtgtgtaaaccgctggtggcgtcggtactggcctcctgctggaatgagtatctgctgaaaccggcgcatttgccgctgcaggacaccaacgatcgctacttcgccaatattcagaaagacggcacttactcggtggtgccgcggatggccgcaggcgaagtgacaccggatgggcttatcgctatcggccagatcgccaaacgttaccagctgtacagtaaagtgaccggaggacagcgtatcgacctgtttggcgcgcggctggagcaactgccggacatctggcgcgaactggcggcggcgggatttgaaaccggacacgcctacggcaaatcattacgcacggtaaaatcctgcgtcggctcgacctggtgccgctatggggttcaggattcaacggggctggcggtaaggcttgaacatcgttacaaggggctgcgcgcgccgcataaaatcaaaatggcggtctcgggctgtacccgcgaatgtgccgaagcgcaaggtaaagacattggggttattgcgactgaaaagggctggaatctttacgtctgcggcaacggcgggatgaaaccgcgccatgcggacctctttgcgagtgaccttgatgacgccagcctaatccgtaccgtcgatcggttgttgatgttttatattcgcaccgccgaccgcctgcagcgcaccagtagctggatggataaccttgaaggcggcgtggcgtatctacgcgaggtgatcttgcacgatagtctcggtatcggcgatgagctggagcaggagatggcgcgggtggtggatagctaccagtgcgaatggcagaccaccctgaacgacccacagcggctggcgctatttcgttcctacgttaacagcgatgaggccgatgaggcggtgcagcggcaaatcctgcgcggtcagccgcagctggtggcgccgcaagggattgggcaaccggccctgccgtcgcggccgtggcaggcgatctgcgatctggacgcgatcccgccgcaggcggggatcggcgcgcgcctcggcgagcggcagattgcgctgttccgcttcggcgagcaagtttatgcgctggataaccaggaacccggcagcgaggccaacgtgttatcgcgcggcattttaggcgacgcgggcggcgagccgatcgttatttcgccgctgtataaacagcggattcgcttgcgagatggtcgcttgtacgacagcggcgagccgtcggtgcgcgcgtggccggtgaagatcgaacagggcaaagtgtgggtcggcaaccaggcattgctgctgcgcgcggaggcgagctga

SEQ ID NO: 206 - DNA, 2607 bp, Klebsiella sp.
  source 1..2607: mol_type = genomic DNA
atgagcgaaacccgaaccacctgtccatactgcggcgtcggctgcggagttatcgcccgcgttgaacccagtggaaccgtcacggttcgcggcgatgaaaaccatcccgctaactttggccgcttgtgcgtaaaaggggcagcgcttggggaaacgacctcgcttagcgggcggttattacaccctgaagtcgatggccgggcggtcggctggccgcaggcgctggcggaggcgggttcacgtctgcgcgacattattgagcgctacggcccgcaggcggtggcgttttacgcttccggtcagctgttaactgaggattattacgccgccaacaagttgatgaagggctttatcggcgccgccaatatcgatacgaattcgcggttatgtatgtcatcggcggtcaccggctataagcgggcgctcggcgcggacgtcgtgccgtgtagctatgaagatgtcgagcagagcgatctggtggtgctggtgggctccaacgccgcctgggcgcatccggttctctatcagcggttggtgcaggcgaaacgcgataatccgcggttgcggatcgtggtggtggatccgcgacgcaccgcgacctgtgatattgccgacgatcatctggcgttggcgcccggtagcgatggcgggctatttgtcggcctgctaaatgccattgccgccagcggtggaatggcggcgggttttcgcgaccagcggcaggcgctgaccatcgccgcgcagtggagcgtggaaaaagcggccgctttctgcggcctggcgccggagcaagtggctcgcttttatcgtgattttatcgctgcaccgcgcgccatcacgctgtacaccatgggaattaatcagtccgccagcggcagcgataagtgcaacgcgattatcaatgtccatcttgccagcgggaagtttgcccgccgcggctgcgggccgttttcgctgaccggtcaacccaacgccatgggcgggcgtgaagtgggggggctggcgacgatgctggcggcgcatatgaattttgagccgcaggacctgcagcgattagcgcgcttttggggcagcgagcgactggcgcagacgccgggactaaccgccgtcgaactcttcgccgcgattggacgcggcgaggtcaaagcggtgtggattatggggaccaatccggtggtctcactgcccgatagccacgcggtgagccaggcgctgtccaggtgtccgctggtgatggtctccgatgtcgccgccaataccgatacggggcgctttgcccatatccgctttccggcgctggcatggggcgaaaaaaacggtaccgtgaccaactcggagcggcggatttcgcgtcagcgggcttttttgccgccgccgggggaggcgaaagccgactggtggatcatttcccgtctcgccgccgaattaggttatgcccgagctttttcatggcaacatccgcatgaggtattttgtgaacacgcggcgctctcagggtttgaaaatgacggtcagcgggcgtttgatattggcgggctggcggacctcagccgggaggcgtgggatgccctgacaccggtgcgctggccggtgagccgcagcgccgcgccctggcggttaacggagggctggcacggcgacggcaggttgcggatggtgccggtgtcgccgcagccgacccgggcgcagaccgaggcattttatccattaatcctcaatagcggacgtattcgcgatcagtggcataccatgacccgcaccggcgaaataccgcggttgatgcagcacattgccgaaccagtcgtcgaggtggcgcctgccgatgcgcgtcgttttcaattacgtgagggagaactggctcgcgtctggtcgcgccacggcgtgatgattgcgaaggtgatagtcagcgaagggcagcgcgccggttcgctctttgtgccgatgcactggaataatcagtttgcccgccagggacgggtcaataatttactggcggcggtcaccgatccgcattccgggcagccggagagcaagcagtcggcggtggcgatagcgccgtggcggccggcgtggcaaggggagctattatctcgcgagcccttagcgttgccatcctcgctgcactggcggcgacgggcggtggtagggcttagccatttgtcgctggccgcggataccggcccgcaaagctggctgaccgagtggtgtcgcaagcagggatggcagttgcaaattgccgagggcggcgggctgtggaacctactggcatggcacgacgggcaattgatgctcggctgctggagcgatattaagccgccgcaaatcgatgctgaatggctgtatacggcgtttcggacgccgccgcagaccgccggcctacgccatgcgttgctcagcggccgtaagggtggcgacagcgcgccgcgtgggcagactatctgtagttgctttagtatcggcgagcggcagatcggcgaggctattgccggcggctgcgtcagcgtagaggcgcttggcgcgaagctcaagtgcggcaccaactgtggttcctgtattccggaactcaaagcgctgctggcggcaaatcaaacccgaacgccggtttga

SEQ ID NO: 207 - DNA, 1389 bp, Klebsiella sp.
  source 1..1389: mol_type = genomic DNA
atgagtcaatcttcaatcccggagaaggctagcagctcagtcatcaccgactggcgtcctgaagatcctgagttttggcaacagcgcggccaccgtgtggcgagccgcaatttatggatctccgttccctgtcttttactggcgttctgcgtctggatgttgtttagcgccgtcgcggtaaacctcaataaagtgggttttcagttcactaccgatcagctgtttatgttgaccgcattgccagcgctttccggcgcgctgctgcgcgtaccttatgcctttatggtcccgctgttcggcggccgccgctggacggcgttcagtaccggtatcatgattattccgtgcgtttggctgggctttgcggtacaggatacttccacgccgtttagcgtgttcgtgattatctctctgctgtgcggctttgccggcgctaactttgcttccagtatggcgaacatcagcttcttcttcccgaaagagaagcagggcggcgcgctgggggttaacggcggccttggtaacatgggcgtcagcgtgatgcagctggttgcgccgctggtagtctcgatttctatttttgccgtttttggcggtagcggtagcgagcagccggatggctcaatgctgtatctggaaaacgccgcgtggatctgggtgccattcctgatcatctttaccctggccgcgtggttctttatgaacgatctgtcggcctcaaaagcgtcgctgagcgaacagttgccggtacttaaacgcctgcatctgtggattatggcgctgctgtatctggcgacctttggttcctttatcggtttctccgccgggtttgcgatgctgtcaaaaacccagttcccggacgtgcagatcctgcactatgccttcttcggtccgtttattggcgcgctggcgcgttcgatgggcggggcgatttccgaccgtctcggcgggacccgcgtgacgctggtgaacttcgtggtgatggctattttctgcgcgttactgttcctgaccctgccaaccaatggtcagggcggcaacttcatcgccttcttcgcggtatttatggtgctgttcctgaccgccgggctgggcagcgcctctaccttccagatgatttccgtgatcttccgtaagcttacgatggaccgcgtgaaagcgcagggcggcagcgaagcgcaagcgatgcgtgaagcggcgacggataccgccgcggcgttgggctttatttcggcgattggcgcgatcggtggtttctttattccgaaagcgttcggtatttcgctggatctgaccggctcgccggccggggcaatgaaagtcttcctcgttttctatattgcctgcgtagtaatcacctgggcggtatacggccgtaaacagaaataa

SEQ ID NO: 208 - DNA, 3744 bp, Klebsiella sp.
  source 1..3744: mol_type = genomic DNA
atgagtaaatttctggaccggtttcgctacttcaaacagaagggtgaaacctttgccgatgggcacggccagcttcttaaaaccaaccgggattgggaggatggataccgccagcgttggcagcatgacaaaatcgtgcgttccactcacggtgtgaactgcaccggctcatgcagctggaaaatttatgtcaaaaatggcctggtcacctgggaaacccagcaaactgattacccgcgtacccgccccgatctgccgaaccatgaaccacgcggctgcccgcgcggcgccagctactcctggtatctgtacagcgctaaccgcctgaaatatccgctgatgcgtaagcgtctgatgaagatgtggcgtgaagcgaaagttcagcatagcgatccggttgaggcatgggcttcaattattgaagatgccgataaagcgaaaagctttaaacaggcccgcggtcgcggcggttttgttcgttcttcatggaaagaagtgaacgaactgattgccgcttcaaacgtctataccgtcaaaacttacggtccagaccgcgtggcaggcttttcgccgatcccggcgatgtcgatggtttcctatgcgtccggcgcccgttacctgtcgctgattggcggtacctgtctgagcttctacgactggtactgcgacctgccgccagcctcgccgatgacctggggcgaacagaccgatgtgccggaatctgccgactggtataactccagctacatcatcgcctggggctccaacgtcccgcagacccgtaccccggacgcgcatttctttaccgaagttcgctacaaagggaccaaaaccgtggcgattactccggactatgccgaaatcgccaagctctgcgatctgtggctggcgccgaagcagggtaccgatgccgcgatggcgctggcgatgggccacgtcatgctgcgtgagttccacctcgataaaccgagccagtacttcaccgattacgtacgtcgctacaccgacatgccgatgctggtcatgctcgaagagcgcgacggctactatgccgctggccgtatgctgcgcgctgccgacctggttgactccctgggccaggagcagaatccggaatggaaaaccgtcgcctttgacgagaagggcgaaatgaccgtacctaacggttctctgggtttccgctggggcgacaaaggcaagtggaacctcgaacagcgcgatggtaaaaccggcgaagaaattgagctgcgcctgagcctgcttggcagccatgatgatatcgccaacgtcggtttcccatactttggcggcgaaggttccgagcatttcaacaaagtcgacctcgaaaacattctgctgcacaaactgccggtaaaacgcctgcagatggccgatggttccaccgcgctggtgactaccgtttatgacctgaccatggcgaactacggcctggaacgcggtctgaacgatgaaaactgcgcgaccagctacgatgacgtcaaggcgtacactccggcctgggcagaaaaaatcaccggcgtatcccgcgcgcacatcatccgtaccgcgcgcgaatttgccgataacgccgataagacccatggccgctcgatgattatcgtcggtgcgggcctgaaccactggttccatctggacatgaactaccgcgggctgatcaacatgctgatcttctgcggctgcgttggccagagcggcggcggttgggcgcactacgttggtcaggaaaaactgcgtccgcagaccggctggcagccgctggcgttcggccttgactggcagcgtccggcacgccacatgaacagtacatcgtacttctataaccactccagccagtggcgctatgaaaccgtcaccgcgcaggaactgctgtcgccgatggcggataaatcccgctacagcggacatctgatcgacttcaacgtgcgcgctgagcgtatgggctggctgccgtcggcgccgcagctgggcgtgaacccgctgcgtattgctgacgaagcgaagaaagccggcatgacgccggtcgattacaccgtgaaatcgctgaaagaggggtctattcgctttgcggccgagcagccggagaacggtaaaaaccatccccgtaacctgtttatctggcgttccaacctgctgggctcctccggtaaaggccatgaatatatgctcaagtacctgctgggtaccgagaacggtattcagggcaaagaccttggcaagcagggcggcgtgaagccggaagaagtcgaatggcgggataacggtctcgacggcaaactggatctggtcgtcacgctcgacttccgtctgtcgagcacctgtctgtactccgacatcgtactgccgaccgcaacctggtacgaaaaagacgatatgaatacctcggatatgcatccgtttattcacccgctgtctgccgccgttgacccggcctgggaatccaaaagcgactgggacatctataaaggtatcgctaagaaattctctgaagtctgcgtaggccacctcggcaaagaaaccgacgtcgtgacgctgccgattcagcacgattccgccgcggaaatggcgcagccgtttgacgttaaagactggaaaaaaggcgaatgtgacctgatcccaggtaaaaccgcgccgcatattattccggtcgagcgtgattatccggcgacctacgagcgtttcacctctatcggcccgctgctggaaaccatcggcaacggcggtaaaggtatcgcctggaatacccagagcgagatggacctgctgcgtaagctcaactacaccaaggcggaaggcccggcgaaaggccagccgaagctggagaccgctattgacgccgctgagatgatcctcaccctggcgccggaaactaacggtcaggtagccgtgaaggcgtggaaagcgctgagcgaatttaccggccgcgaccatgcgcatctggcgctgaacaaagaagacgagaagatccgttttcgcgatatccaggcgcagccgcgtaagatcatctccagcccgacctggtctggcctggaagacgaacatgtctcctataacgccggttataccaacgttcacgagctgatcccatggcgtacgctgtccggccgtcagtcgctgtatcaggatcaccagtggatgcgcgactttggcgaaagcctgctggtctatcgtccgccgattgacacccgttcggtgaaagcggtgatgggcgagaagtccaacggcaataaggagaaggcgctgaacttcctgacgccgcaccagaagtgggggattcactctacttatagcgataacctgctgatgctgactctgtcgcgcggcgggccgatcgtctggatgagcgaagcggatgcgaaagatctgggtatcgaggataacgactggatcgaagtcttcaacgccaacggcgccctgacggcgcgtgcggtagtgagccagcgcgtaccggcaggaatgaccatgatgtaccacgcgcaggaacgtatcgttaacctgccgggttctgaagttaccggtcagcgcggggggatccacaactcggttacccgtattacgccaaaaccgacccatatgatcggcggctacggccacctggcgtacggctttaactactatggcaccgtcggttccaaccgcgatgagtttgttgtggtacgtaagatgaagaacattaactggttagacggcgaaggtaatgaccaggtacaggagagcgtaaaatga

SEQ ID NO: 209 - DNA, 1536 bp, Klebsiella sp.
  source 1..1536: mol_type = genomic DNA
atgaaaattcgttcacaagtcggcatggtgctgaatctcgataaatgcatcggctgtcacacctgctccgtaacctgcaaaaacgtctggaccagtcgtgaagggatggagtacgcctggtttaacaacgtagaaagtaagccgggcgtcggtttcccgaacgactgggaaaaccaggaaaaatggaaaggcggctggatccgtaaaatcaacggtaaactgcagccgcgcatgggcaaccgcgcgatgctgctgggtaaaatcttcgccaacccgcatctgccgggaatcgatgattactacgagccgtttgattttgactaccagaatctgcataacgcgccggaaagcaaacatcagccgatcgctcgtccgcgttcgctgattaccggccagcggatggacaaaattaccagcggcccgaactgggaagagatcctcggcggcgagtttgaaaaacgcgccaaagaccagaacttcgaaaacatgcagaaggcgatgtacggccagttcgaaaacaccttcatgatgtatctgccgcgcttatgcgagcactgcctgaacccggcgtgcgtcgcgacctgcccgagcggtgccatctataagcgtgaagaagacggtattgtcctgatcgaccaggataagtgccgcggctggcgtatgtgcatcaccggctgcccgtacaagaagatctatttcaactggaagagcgggaaatccgagaaatgcatcttctgctacccgcgtattgaatccgggatgccgacggtgtgctccgaaacctgtgtgggacgtattcgctatctcggtgtcctgctctacgacgcggacgcgattgaaaacgcagccagcaccgagaacgagaaagatctgtatcagcgtcaactggacgtcttcctcgatcctaacgatccaaaagtgattgaacaggcattaaaagatggtgtaccgcagggcgtaattgaagccgcgcagcagtcgccggtttacaaaatggcgatggactggaagctggcgctgccgctgcacccggaatatcgcaccttgccaatggtctggtacgtgccgccgctgtcgccgattcagtccgccgccgatgcgggcgaactgggtagcaacgggatcctgccggatgtagaaagcctgcgtattccagtccagtatctggcgaacctgctgaccgcgggcgatacccagcctgtactgctggcgctgaaacgtatgctggcgatgcgccacttcaaacgtgcggaaaccgtcgacggcatcgtcgatacccgcgcgctggaagaggtcgggctgagcgaagcgcaggcgcaggagatgtaccgttatctggctatcgccaactacgaagatcgtttcgtggtgccgagcagccatcgcgaacaggctcgcgatgccttcccggagaagagcggttgcggctttaccttcggcgatggttgccacggctctgacagcgaattcaacctgtttaacagccgtcgcattgacgccattgacgttaccagcaaaacggagccgcacgcatga

SEQ ID NO: 210 - DNA, 711 bp, Klebsiella sp.
  source 1..711: mol_type = genomic DNA
atgattgaactcgtgattgtgtcgcgtctgctcgaataccctgacgctgccttatggcagcatcagcaggagatgttcgaggctctcgcgtcatcggaaaaactcgccaaagaagatgcccaggcgctgggcgttttcctgcgcgatttagtcgctcaggatccgctggacgcccaatcggcgtatagcgagctgtttgaccgcggccgcgccacctcgctgctgctgtttgaacatgttcacggcgagtcccgcgatcgcgggcaggcgatggtcgacctgatggcgcagtacgagcgccacggtctgctgctggatagccatgagctgccggatcacctgccgctgtaccttgagtatctggcgcagttgccggaagaggaagcgcttggcggcctgcgggacgttgcgccaatccttggcctgctcagcgcgcgtctgcaacagcgcgagagccgctatgcggtgttgttcgagctgctgttgaagcttgccaatactcaggtcgatagccagaaagtggcggagaagattgccgacgaggcccgcgacgatacaccgcaggcgctggatgccgtctgggaagaagagcaggtcaaattctttgccgaccagggctgcggcgagtcggaaatctccgctcaccagcgccgttttgccggagccgtggccccgcaatatttgaatatctctaacggaggacagcactaa

SEQ ID NO: 211 - DNA, 678 bp, Klebsiella sp.
  source 1..678: mol_type = genomic DNA
atgcacttcctgaatatgttcttctttgacatctatccgtacattgcgggttcagtgtttcttattggcagctggctgcgctacgactacggccagtacacctggcgcgctgcctccagtcagatgctggatcgtaaagggatgaacctggcgtcaaacctgttccatatcgggatcctgggtatttttgccggtcacttcctcggtatgctgaccccgcactggatgtatgagtccttcctgccgatcgacgtgaagcagaaaatggcgatgtttgccggcggggcgtgcggcgtgatgacgttggtcggcggtttactgctgctcaagcgccgtctgctgagcccgcgcgttcgtgctaccaccaccacagcggatatcctgatcctctcgttgctgatggtgcaatgcgcgctgggtctgctgaccattccattctccgcccagcatatggacggtagcgaaatgatgaaactggtcggctgggcgcagtcggtggtgaccttccacggcggagcttcgcagcatctcgatggcgtagcttttgtcttccgagttcacctggtgctggggatgacgctgttcctgctgttcccgttctcgcgcctggttcacatctggagcgcgccggtcgagtacctgacgcgcaaataccagattgtgcgcgcgcgtcgctag

SEQ ID NO: 212 - DNA, 1680 bp, Klebsiella sp.
  source 1..1680: mol_type = genomic DNA
atggacaaacagtatccgcagcgccagtgggcgcacggcgccgatctggtcgtcagccaactggaagcgcaaggcgtacggcaggtcttcgggatccccggcgctaaaatcgataaggttttcgactcgttgctggactcctcaatccgcattattccggtacgtcacgaggccaacgccgcctttatggccgccgcggtcgggcgcattaccggcaaagcgggcgtcgcgctggtgacctccggacccggttgttccaacctgataaccgggatggccaccgccaatagcgaaggcgacccggtggtggcgctgggcggcgcggtcaaacgcgcggataaagccaaacaggtacaccagagtatggacacggtggcgatgttcagcccggtcaccaaatacgcggtagaagtgacctcgccggatgcgctggcggaagtggtttctaacgcttttcgcgccgccgagcagggtcgcccgggcagcgccttcgtcagtctgccgcaggatgtggtcgatggtccggtgaccggcaaagtcctacccgccagcagcgcgccgcagatgggcgccgcgcctgacgaggcaatcaatcaggttgcgaagttgattgcccaggcgaagaatccggtgttcctgcttggattaatggccagccagacggaaaacagcgccgcgctgcatcgtttgctggaaaccagccatattccggtcaccagcacctatcaggccgccggggcggtcaatcaggataacttctcgcgcttcgccgggcgcgtcgggctgtttaacaatcaggccggtgaccgcttattgcaactggccgacctggttatctgcatcggctatagcccggtggaatacgaaccggcgatgtggaacagcggcaacgcgacgctggtccacatcgacgtactgcccgcctatgaagagcgtaactacacgccggatgtcgagctggtgggcgacatcgccggcacgctgaacaagctggcgcaaaatatcgatcatcggctggtgctctcgccgcaggccgctgaaatcctccacgaccgccagcatcaacgcgaactgcttgaccgccgcggagcgcagttgaatcagtttgccctgcacccgctgcgcatcgttcgcgccatgcaggatatcgtcaacagcgacgtcacgctgacggtcgatatggggagcttccatatctggatcgcccgctatctctacagcttccgcgcccgtcaggtgatgatctccaacggtcagcagaccatgggcgtcgccctgccgtgggccatcggggcctggctggtcaatccgcagcgcaaagtggtctcggtctccggcgatggcggttttctgcaatccagcatggagctggaaacggcggtccgcctgaaagccaacatcctgcatcttatctgggtcgataacggctacaacatggtcgctatccaggaagagaaaaaatatcaacgcctgtccggcgtcgagttcggtcctatggattttaaagcctatgccgaatccttcggcgcgaaagggtttgcggtggaaagcgctgaggcgctggagccgacgctacgcgcggcgatggacgtcgacggcccggcggtggtcgccatccccgtggattaccgtgataacccgctgctgatgggccagctacacctgagtcaaattctttaa

SEQ ID NO: 213 - DNA, 780 bp, Klebsiella sp.
  source 1..780: mol_type = genomic DNA
atgaatcatgcttcagattgcacctgcgaagagagtctgtgtgaaacgctacgcgcgttttccgctcagcatcccgatagcgtgctgtatcaaacttcgctgatgagcgccctgctcagcggcgtctacgaaggtaccaccaccattgcggacctgctgaagcacggtgatttcgggctcggcacttttaatgaactcgacggcgagctgatcgcgtttagcagccaggtttatcaactgcgtgccgacggcagcgcgcgtaaagcgcgtccggaacagaaaacgccgtttgcggtgatgacctggtttcagccgcagtaccgtaaaacctttgaccatccggtcagccgccagcagctgcatgaggttattgaccagcaaattccttccgacaatctgttctgcgcgctgcgaatcgatggtcatttccgccacgcccatacccgcaccgtgcctcgtcagacgccgccctaccgggcgatgaccgacgtgctcgacgatcagccggttttccgctttaaccagcgtgacggcgtactggtcggttttcggaccccccagcatatgcagggaattaacgtcgccggctatcacgaacacttcattaccgatgaccgccagggcggcggccacctgctggactaccagctcgaccatggggtattgaccttcggcgaaattcataagctgatgatcgaccttcccgccgacagcgcgttcctgcaggccaatttgcatcccgataatctcgatgccgccatccgttcagtagaaagttag

SEQ ID NO: 214 - DNA, 2391 bp, Klebsiella sp.
  source 1..2391: mol_type = genomic DNA
atggcagaaacaaaatctcaacaatcacggttacttgtcacgctgacggcgctgtttgccgccttctgtggcctgtatctgttaatcggtggggtatggctggccgctattggcggttcctggtactaccctatcgcaggcctggtgatgctggccgtcaccgttatgctgttccgcggcaagcgcgctgcgctgtggctgtacgccgccctgctgctggcaaccatgatttggggggtatgggaagtcggtttcgacttctgggcgctgacgccgcgtagcgacatcctggtcttcttcggcatctggctgattctgccatttgtctggcgtcgtctgccggtcccttccgccggtgccgttggcggtctggttatcgccctgctgattagcggcgggatcctgacctgggccggtttcaacgatccgcaggaagtgaacggtacgctgagcgctgacgcgacgccggctgcgccgatttctaccgtcgccgatagcgactggccggcttatggccgcaaccaggaaggccagcgttattcaccgctgaagcaaattaataccgataacgtgaagaacctgaaggaagcctgggtattccgcaccggcgacctgaagcagccgaacgatccgggtgaaatcactaacgaagtgacgccgattaaagttggcgacatgctgtatctgtgtaccgcgcaccagcgtctgttcgcgcttgacgcggccaccggtaaagagaagtggcattttgacccgcagctgaacgccgatccgtcgttccagcacgtgacctgccgtggcgtctcctatcatgaagccaaagcagataacgcgcctgccgacgtcgtcgccgactgcccgcgccgtattattctgccggtcaacgatggccgtctgttcgcggtgaacgccgacaacggtaagctgtgcgaaacctttgccaacaaaggcattctcaacctgcaaaccaatatgccggtaaccacgccgggtatgtatgaaccgacgtcgccgccgattatcaccgataagactatcgtcattgccggcgcggtaaccgataacttctcaacccgcgagccatcaggcgtaatccgcggctttgatgtgaataccggtaaactgctgtgggccttcgacccgggtgcgaaagaccctaatgcgatcccgagcgatgagcatcacttcacactcaattcacctaactcctgggcgcctgccgcctatgacgcgaagctggatttagtctatctgccgatgggcgttaccacgccggatatctggggcggcaatcgcacgccggaacaggaacgttacgccagctctatcgtagcgctgaacgcaaccaccgggaaactggcatggagctaccaaaccgtacaccacgatctgtgggatatggatatgccgtcgcagccgacgctggccgacattgatgtgaacggtaaaaccgtgccggtgatttacgctccggccaaaaccggcaacatcttcgtgctggatcgccgtaatggcgaactggtggtcccggcgccggaaaaaccggttccgcaaggtgcggcgaaaggcgattatgttgctaaaacccagccgttctccgagctgagcttccgtccgaagaaagacctgaccggcgcagatatgtggggcgccaccatgtttgaccaactggtgtgccgcgttatcttccatcagatgcgctatgaaggtatcttcactccaccgtctgaacagggcacgctggtcttcccggggaacctggggatgtttgaatggggcggtatctccgtcgatccaaaccgtcaggtggctatcgccaacccgatggcgctgccgttcgtctctaaattgatcccacgcggcccgggcaacccgatggaaccgccaaaagacgcgaaaggctctggtaccgagtccggcgttcagccgcagtatggcgtcccgttcggcgtcacgctgaacccgttcctgtcgccgtttggcctgccgtgtaaacaaccggcatggggttatatctcggcgctggatctgaagaccaacgaagtagtgtggaaaaaacgcatcggtacgccgcaggatagtctgccgttcccgctgcctgtcccgctgccattcaatatgggtatgccgatgctgggcggaccgatctcaaccgccggtaacgtgctgtttatcgccgccaccgccgataactacctgcgcgcttacaacatgagtaatggtgaaaaactgtggcaggcgcgtctgcctgcaggtggccaggcgacgccgatgacctatgaagtgaatggcaagcagtacgttgtcatctctgccggcggtcatggctcgttcggtactaaaatgggcgactacatcgttgcctatgcgttaccggatgacgcgaaataa

SEQ ID NO: 215 - DNA, 861 bp, Klebsiella sp.
  source 1..861: mol_type = genomic DNA
atggccgagagtaatcagtggcatgagacgctgcacgaccattttggtcaatattttaccgttgataacgtactgtaccatgagaagaccgatcatcaggatttaatcatcttcgaaaatgcggcatttggccgcgtgatggcgcttgatggcgtggtgcaaaccaccgagcgcgatgagtttatttaccatgaaatgatgacccacgttccgctgctggcccatggtcacgcaaagcacgtgctgatcatcggcggcggcgatggcgcgatgctgcgtgaagtctcccgccaccaacatatcgaaaccattaccatggtggaaatcgacgccggagtggtatcgttctgccgtcagtaccttccaaaccataacgccggcgcctatgacgacccgcgctttacgctggtgattgatgacggcgtgaacttcgtcaaccaaacgtcacaaacttttgatgtcattatctccgactgtactgacccgatcggccccggtgagagcctgtttacctcggcgttttatgctggctgtaaacgttgcctgaaccccggcggaattttcgttgcccagaacggcgtaagcttcctgcagcaggatgaagcggtaggcagccatcgcaaactcagccactatttcaccgatgtcagcttctaccaggcggcgatcccgacctactatggcggtatcatgacctttgcctgggcgaccgataatgaagccctgcgtcatttgtcgacggaaattattcaggcccgcttccatagcgccgggctgaaatgccgttattacaatccggcaattcataccgcggctttcgcgctaccgcaatacctgctggatgcgctgaccgccagctaa

SEQ ID NO: 216 - DNA, 795 bp, Klebsiella sp.
  source 1..795: mol_type = genomic DNA
ttgaaaaagcttaagctgcacggcttcaacaacctgactaaaagcctgagtttttgtatttacgatatctgctacgctaaaaccgccgaagagcgcgatggctatatagcctatatcgatgaactctataatgccaatcgcctgacggaaatcctctccgagacctgttcaatcattggcgccaacatactcaacatcgcacgccaggattatgaaccgcagggcgcaagcgtcaccattttggttagcgaagagcctatcgatccgcagcttatcgatcagagcgagcatccgggcccgctgcctgagaccgtggtcgcacatctcgataaaagccacatctgcgtgcatacctatccggaaagccatccggaaggcggtctgtgcaccttccgcgccgatattgaagtctccacctgtggagtgatttcaccgctgaaggcactgaactacctgattcaccaactggaatccgatatcgtaaccattgattatcgcgtgcgtggtttcacccgtgatatcaacggtatgaagcattttatcgatcatgaaatcaactctattcagaacttcatgtcagaagatatgaagtcgctctatgacatggtggacgtgaacgtctatcaggaaaatatgttccataccaaaatgatgctgaaagagttcgatctgaaacactacatgttccacaccaaaccggaagagttaagcgaacaagagcgcaaggtgattaccgacctgctgtggaaagagatgcgggaaatttattacgcccgcaatatccctgcggtgtaa

SEQ ID NO: 217 - DNA, 492 bp, Klebsiella sp.
  source 1..492: mol_type = genomic DNA
atgcgccggatattatctgtattactggagaacgaatcgggagcattatcccgggtgatcggcctcttttcgcagcgcggctacaatattgaaagcctgacggtagcgccaaccgacgatccgacgttgtcccgcatgaccatccagacggtgggcgacgaaaaagccattgagcagatcgaaaagcaattacataagctggtggacgtgctgcgggtcagcgaactgggtcagggctcccacgtcgaacgtgaaatcatgctggtgaaagtgcaggccagcggttatggccgcgaagaggtgaaacgcaacaccgagatcttccgcgggcagatcattgacgtcacgccatctatttataccgtgcagttggccggaaccagcgataagctggatgcgttcctggcttctttacgcgatgtcgcgcgcattgttgaagtggcgcgatccggagtagtcggcctgtcgcgcggcgataaaatcatgcgctaa

SEQ ID NO: 218 - DNA, 1725 bp, Klebsiella sp.
  source 1..1725: mol_type = genomic DNA
atggagatgttgtcaggagccgagatggtcgtccaatcgcttgtcgatcagggcgtcaagcaagtattcggctatcccggaggcgcggtcctcgatatctatgatgcattacataccctcggcggcattgaccatgtgctggtccgccatgagcaggcggcggtgcatatggccgacggactggcgcgcgcaaccggcgaagtcggggtggtgctggtgacctccggcccgggagcgactaacgctattaccggcattgcaacggcttacatggattctattccgctggtcattctttccgggcaggtcgccacctcgctgattggctacgatgccttccaggagtgcgatatggtcggtatctcgcgcccggtggttaaacacagcttcctcgtgaaacagactgaagatatccccgggattttaaagaaagccttctggctggcggccagcggccgaccggggccggtagtggtcgatttgccgaaggatattctcaatccggcgaaaaagctgccttatgtttggccggatgcggtcagcatgcgttcctataacccgacaaccagcggtcataaagggcagatcaaacgtgcgttgcaaacgctggtggcggcgaaaaaaccggtcgtgtacgttggcggcggggcaatcaattcgcagtgcgaagcacagctgcgtacgctggtcgaaaagctgaagctaccggtggtctcttcattaatgggtttaggcgcttttccggcgactcatcagcaggcgctggggatgttggggatgcacggtacctatgaagccaacatgaccatgcatcattccgacgttatttttgcagtcggcgtccgctttgacgatcgcaccaccaacaatttggctaagtattgcccgaacgccacggtgctgcatattgatatcgatcccacgtcgatctccaaaaccgtcccggcagatgtgccgatcgtcggcgacgcgcgccaggtgcttgaccagatgtttgatctgcttgagcaggaagagagccagcaaccgctggatgagatccgcgactggtggcagcagatcgaacagtggcgttcccgtcactgtctgcaatacgacacgcaaagcggcaagatcaaaccgcaggcggtgattgaggctatctggcgcctgaccaatggtgatgcttacgtgacttccgacgttggacagcatcagatgttcgccgcgctctattatccattcgataaaccacggcgttggattaactccggcggcctcggcacgatgggctttggcctgccagcggcactgggcgtgaagatggcgctaccgcaagaaaccgtgatctgcgtgaccggcgatggcagcatccagatgaacattcaggagctttccaccgccctgcagtatgaactgccggtgctggtgctgaacctgaataaccgctacctcggaatggtaaagcagtggcaggatatgctctattctggccgccattcgcagtcctatatggaatcgctgccggacttcgttcgcgtcgccgaggcttatggccacgttggtattcgtatcagcgagccgcaagagctggaagcgaagctggcggaagccctggagcaggtgcgtaataatcgtctggtgttcgttgatgttacggttgatggcagcgagcatgtttatcccatgcagatccgcggcggcggcatggacgaaatgtggttgagcaaaacggagagaacctga

SEQ ID NO: 219 - DNA, 531 bp, Klebsiella sp.
  source 1..531: mol_type = genomic DNA
atgagcttactcaacgtacctgcgggcaaagagctgccggaagacatctacgtcgttatcgaaatcccagctaacgcagacccaatcaaatacgaagttgacaaagagagcggcgcactgttcgttgaccgtttcatgtccactgcgatgttctatccgtgcaactacggctacatcaaccacaccctgtccctggacggcgacccggttgacgtgctggtcccgactccgtacccgctgcaaccaggctcagttatccgctgccgtccggttggcgtactgaaaatgaccgacgaatccggtgaagatgccaagctggttgcggtaccgcacaccaagctgagcaaagaatacgatcacatcaaagatgtgaacgacctgccggaactgctgaaagcccagatcactcacttcttcgagcactacaaagatctggaaaaaggcaagtgggttaaagtcgacggttgggacaacgctgaagcggctaaagctgaaatcgttgcttccttcgagcgcgctaaacagaagtaa

SEQ ID NO: 220 - DNA, 1263 bp, Klebsiella sp.
  source 1..1263: mol_type = genomic DNA
atgaacacgctgcgctgtgacattttaatcctcggcgccggccccgcaggggtggcggccgcgctctccgccgccgcctgcgataaacaggtgattattctcgacgataatcccgcccctggcgggcaaatctggcgtgccggtccgcaggcgacgcagcccgccctggcccggcactaccgcgacgccatcgccgcgtctgccgccatccggttagtgaacggcgcgcggctgattgcccgcccctccgcgtacagtgtgctgtttgaaaccgccgacggcggtggtgtggtttactggcagaaactgatcctctgctgcggcgcgcgcgagctgtcgctgccctttcccggttggaccttacccggcgtgaccggcgcgggcggcctgcaggcgcaaatcaaacagggccttgcgctgaaaggagaaaaggtggtgattgccggcagcggtccgctactgctggcggtggcggatacggtgaacaaggccggaggcgaagtgacgaatatcattgagcaagcgccgctaccggcactgctgcgcttcgccggtgggctgtggcgctggccgcagaagctgcgccagttagcgatgctggctccgaaaggctatcttagcggcacgcaggtgatccgcgctcacggcacaacgcggctggaagccgttacgttgcgccagcgcggcgacgagcgaactatcgcctgcgatcggctggctatcggctacgggctgatacccaatatcgagacggcgctgctgtttggctgcgccacggcgcaggaggcgatacaggttaaccgctggcagcagaccagcatcgcggatatctacgccgccggcgagtgcaccggtttcggcggtagcgaactggcgctggcggagggcgaaatcgccggttttgccgccgccggggccagccatcaagcgcaggcgttatttgcccgccgcgcccgctggcagcgcttcgccgacgccataaaccgcgccttccggctggcggaatctttgaaaaacgcggcgacgccggagagcctgctgtgccgctgcgaggatgtgcgctgcggcgatgtggcggcagccggaggctggccgcaggccaaacttacccagcgctgcggaatgggcgcctgtcaggggcgcacctgcgccgccagcgcccgctggttatatggctggccgctgccgcagccgagagaaccgctggcccctgcccgcgcggaaacgcttattgccctcgccaggttgagcgccgagccgtaa

SEQ ID NO: 221 - DNA, 264 bp, Klebsiella sp.
  source 1..264: mol_type = genomic DNA
atgaatgccacgctcactatcatcgttgatggcgaagcgctgacggtgccggaagggatcagcgtggcggcggcgctggcgttgaccggcgaccccaccacccgccaggcggttaacggcggcctccgcgcgccgttttgcggcatgggcgtctgccaggagtgccgggtcaccgtcgatggcctacgggtgctggcctgccagaccctgtgccggtccgatatgcaaatagaaaggagccgcgatgaacacgctgcgctgtga

SEQ ID NO: 222 - DNA, 1110 bp, Klebsiella sp.
  source 1..1110: mol_type = genomic DNA
gtgaagcagtgcgacgtcatcgtgatcggcgccgggatcatcggcgccgcctgcgcgtggcagctggcgaagcgggggcagagcgtgacgcttatcgatgacggtcagcctggcgcgacggcggccggtatgggccatctggtttgcatggatgacgaccccgccgagcttgcattatcggcatggtcgctggaacgctggcgcgctatcacgccacggatgcccgacaattgcgcctggcgcggctgcggtacgctgtggctggcggaaagcgaagaagagatggccggggctggcgacaaacagcggcggatggccggccatcaggtgcacagcgaactccagaccccgcagcagatcgccgggcgcgagccgctgctgcgcgacgggctggccggcggcctgtgggtgccaggcgatggcatcgtctacgcgccgaacgtcgcccgctggctgattaccgatgccggaaaccatcttacctgcctgcgcgatagcgtacagacgattgacgagccgcaggtgctgctcgccagcggcaaacggctacaggcgcgggcgatcgtggtggcctgcggccttgaagctaacgcgctgttagcggaaaactggctgcgaccgaaaaaaggccaactggcgattaccgatcgctatgggccgcaggtgcatcaccagctggttgagctgggttatggcgccagcgcccacggcggcggcacctcggtggcgtttaaccttcagccgcggccaaccggccagctgctaattggctcttcgcgtcagtttgataaccgcgagcgcgagttggatctgcccttgctggcgcagatgctcgatcgcgcccgccacttcgtgccggcgctggcgacgttgaatatcatccgctgctggagcggcctgcgcgccgcctctccggatggcaatccgttgatcggcccgcaccccacccgccgtggtttatggctagcgctgggtcatgaagggctgggcgtcaccactgccccggcctcggctgaactgctggcggcgcagatcctcgatgaacgctgcccattggctcccgacgcctggctcccggctcgtttatctcaacaggaggcgatcgcatga

SEQ ID NO: 223 - DNA, 258 bp, Klebsiella sp.
  source 1..258: mol_type = genomic DNA
atgatgcaacatcaggtcgctttacaggctcgcttcaaccccgaaaccttagaacgcgtgctgcgcgtggtgcgccatcgcggttttcaaatttgctcaatgaatatggaaaccgcgtcggatgcgcaaaacataaatatcgagctgaccgttgccagccagcggcccgtcgaattactgtttagtcagttacgcaaactggtcgacgtcgcctgcgtcgagatccagcaacccacatcacaacaaatccgcgcctga

SEQ ID NO: 224 - DNA, 1647 bp, Klebsiella sp.
  source 1..1647: mol_type = genomic DNA
atgaacggggcgcagtgggtggtacatgctttgcgaacacagggggtcgacacggtatttggctatccgggtggcgcgattatgccggtttacgatgctttgtatgacggcggcgtggaacacctgctgtgtcggcacgagcaaggcgccgcaatggccgccatcggttatgcccgcgcgaccggcaaaactggtgtttgcatcgccacttccggtcctggcgccaccaacctgatcaccggtttggctgacgcgttacttgattctgtacctgttgtcgccatcaccggtcaagtggcggcgccgtttatcggcaccgatgcttttcaggaagtggacgttctcggtttgtcgctggcctgcaccaaacacagtttcctcgtgcagtcgttggaagagctgccgcgcgtcattgcggaagctttccaggtggcaaactcaggccgtcctggcccggtactggttgatattccaaaagatatccagttggctaaaggcgaattagatccgcatttctccaccgtccctgatgatgttgagttcccgcacacacaagtcgagcaggcgttagcgatgcttgcgcagtcccacaagccaatgctgtacgtgggtggcggtgttggaatggcacaggcggtaccggccgtgcgcgaatttctggcggtgacgcagatgccggtaacctgcactctgaaagggttgggcgccgtcgccgcggattatccgtattaccttggcatgctgggtatgcacggaaccaaggcagcaaacctggcggtgcaggagtgcgatttattaatcgccgtcggcgcccgttttgatgaccgggttaccggcaagctgaatacctttgccccgcacgccaaagtaatccatatggatattgacccggccgagctgaacaaactgcgccaggcgcacgtcgccttaaccggagatttaaacgccatgctgccggcgttgcagcagccgttggccatcgatgcgtggcgcgagcgcaacgcgcagctgcgcgcggagcacgcctggcgttacgatcatcccggcgaggcaatctacgcgccgctgttgctcaagcagctttccgatcgcaaaccggcggattgcgtcgtgacgaccgatgtcggccagcaccaaatgtggtcggcccagcacatgacctatacccgcccggaaaacttcatcacttccagcggcctcggcaccatgggttttggtctgccggcagccgttggcgcgcaggtggctcgcccggacgatacggttatctgtatctccggcgatggctctttcatgatgaacgtgcaggagctgggcaccgttaagcgcaagcaattaccgttgaaaatcgtgctgctggataaccaacgtttaggcatggttcgccagtggcagcagctgtttttccaggagcgttacagcgaaaccacgcttaccgataatcctgattttcttacgctggccagcgcttttggcattccaggccagcacatcacccgtaaagaccaggttgaagcggcactcgacaccatgctttcgagccaggggccatacctgcttcatgtctcaatcgatgaacttgagaatgtctggccgttggtgccgcccggcgccagtaatgcagaaatgctggagaaattatcatga

(19) United States
(12) Patent Application Publication
(10) Pub. No.: US 2026/0026508 A1
(43) Pub. Date: Jan. 29, 2026

(54) Seed Treatment Formulations and Methods of Use

(71) Applicants: Puna Bio Corporation, San Francisco, CA (US); Consejo Nacional de Investigaciones Cientificas y Técnicas (CONICET), Buenos Aires (AR)

(72) Inventors: Elisa Violeta Bertini, San Miguel de Tucumán (AR); carolina Belfiore, Yerba Buena, Tucumán (AR); Maria Eugenia Farías, Lules, Tucumán (AR); Ana Paula Santos, San Miguel de Tucumán (AR)

(21) Appl. No.: 18/993,447

(22) PCT Filed: Jul. 10, 2023
(86) PCT No.: PCT/US23/27283

§ 371 Date: Jan. 10, 2025

Related U.S. Application Data
(60) Provisional application No. 63/368,135, filed on Jul. 11, 2022.
(60) Provisional application No. 63/476,280, filed on Dec. 20, 2022.

(51) Int. Cl. A01N 63/22; A01N 63/20; A01P 3/00; A01P 21/00; C12N 1/20; C12R 1/01; C12R 1/085; C12R 1/22

(52) U.S. Cl. CPC A01N 63/22; A01N 63/20; A01P 3/00; A01P 21/00; C12N 1/205; C12R 2001/01; C12R 2001/085; C12R 2001/22

(57) ABSTRACT

Provided herein are seed treatment formulations that provide improved growth and yield in the plants grown from seeds treated with such formulations, as well as methods of use.

### CROSS REFERENCE

This application claims the benefit of U.S. Provisional Application No. 63/368,135, filed Jul. 11, 2022, and U.S. Provisional Application No. 63/476,280, filed Dec. 20, 2022, each of which is incorporated herein by reference in its entirety.

### BACKGROUND

By 2050, the world population is expected to reach 9.8 billion while more than 500 million hectares of extended wild lands will change to cropland (IRP. 2017). Under current conditions, agricultural production has to face severe challenges due to climate change with extreme weather events and emerging pathogens, while farmers globally have cope with decreasing yields and low operating margins mainly due to the latter (GAP 2017: Sessitsch et al., 2018). When considering both, the expected worldwide population increase and the environmental damage, it is clear that in the next decade it will be a significant challenge to greatly increase agriculture and food production in a sustainable and environmentally friendly manner.

### SUMMARY

Provided herein, in certain aspects, are compositions comprising a microorganism and at least one seed formulation component. In some embodiments, the microorganism is selected from Klebsiella, Bacillus licheniformis, Bacillus cereus, Exiguobacterium, or a combination thereof. In some embodiments, the seed formulation component is an adjuvant, a stabilizer, an additive, or a combination thereof. In some embodiments, the seed formulation component is selected from one or more of polyvinylpyrrolidone (PVP), gum Arabic, and Xanthan gum. In some embodiments, the seed formulation is a nutrient. In some embodiments, the composition further comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the microorganism is selected from Klebsiella aerogenes, Bacillus licheniformis, Bacillus cereus, Exiguobacterium undeae, or a combination thereof. In some embodiments, the Klebsiella aerogenes is a strain CK1, or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%. 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%, 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%. 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In another aspect, there are provided, compositions comprising a microorganism isolated from a plant growing in the high desert and at least one seed formulation component. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes. Bacillus licheniformis. Bacillus cereus, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the seed formulation component is an adjuvant, a stabilizer, an additive, or a combination thereof. In some embodiments, the seed formulation component is selected from one or more of polyvinylpyrrolidone (PVP), gum Arabic, and Xanthan gum. In some embodiments, the composition comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction; dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%. 99%, or 100% identity to a nitrogen pathway signature gene set forth in Table 25. In some embodiments the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%. 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In another aspect, there are provided treated seeds comprising a plant seed and any one of the compositions provided herein. In some embodiments, the compositions comprise a microorganism and at least one seed formulation component. In some embodiments, the microorganism is selected from Klebsiella, Bacillus licheniformis. Bacillus cereus, Exiguobacterium undeae, or a combination thereof. In some embodiments, the seed formulation component is an adjuvant, a stabilizer, an additive, or a combination thereof. In some embodiments, the seed formulation component is selected from one or more of polyvinylpyrrolidone (PVP), gum Arabic, and Xanthan gum. In some embodiments, the composition further comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1.x 1010 CFU/ml. In some embodiments, the microorganism is selected from Klebsiella aerogenes, Bacillus licheniformis, Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp, or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%. 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction; dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%. 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises a microorganism isolated from a plant growing in the high desert and at least one seed formulation component. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes Bacillus cereus, Bacillus licheniformis, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the seed formulation component is an adjuvant, a stabilizer, an additive, or a combination thereof. In some embodiments, the seed formulation component is selected from one or more of polyvinylpyrrolidone (PVP), gum Arabic. and Xanthan gum. In some embodiments, the composition comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%. 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%, 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In another aspect, there are provided plants grown from treated seeds comprising a plant seed and any one of the compositions provided herein. In some embodiments, the composition comprises a microorganism and at least one seed formulation component. In some embodiments, the microorganism is selected from Klebsiella, Bacillus licheniformis, Bacillus cereus, Exiguobacterium undeae, or a combination thereof. In some embodiments, the seed formulation component is an adjuvant, a stabilizer, an additive, or a combination thereof. In some embodiments, the seed formulation component is selected from one or more of polyvinylpyrrolidone (PVP), gum Arabic, and Xanthan gum. In some embodiments, the composition further comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the microorganism is selected from Klebsiella aerogenes. Bacillus licheniformis. Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%. 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%, 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%. 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises a microorganism isolated from a plant growing in the high desert and at least one seed formulation component. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes. Bacillus licheniformis. Bacillus cereus, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the seed formulation component is an adjuvant, a stabilizer, an additive, or a combination thereof. In some embodiments, the seed formulation component is selected from one or more of polyvinylpyrrolidone (PVP), gum Arabic, and Xanthan gum. In some embodiments, the composition comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction; dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%. 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%. 99%, or 100% identity to a phosphate solubilization signature gene set forth in Table 26. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In another aspect, provided herein are methods of controlling fungal growth, the method comprising contacting a plant seed to any composition provided herein; and germinating the plant seed under a condition capable of exposing the plant seed to a fungus, whereby the seed treatment reduces growth of the fungus on or around the plant seed. In some embodiments, the composition comprises a microorganism and at least one seed formulation component. In some embodiments, the microorganism is selected from Klebsiella, Bacillus licheniformis, Bacillus cereus, Exiguobacterium undeae, or a combination thereof. In some embodiments, the seed formulation component is an adjuvant, a stabilizer, an additive, or a combination thereof. In some embodiments, the seed formulation component is selected from one or more of polyvinylpyrrolidone (PVP), gum Arabic, and Xanthan gum. In some embodiments, the composition further comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the microorganism is selected from Klebsiella aerogenes. Bacillus licheniformis. Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%. 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%. 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises a microorganism isolated from a plant growing in the high desert and at least one seed formulation component. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes. Bacillus licheniformis. Bacillus cereus, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the seed formulation component is an adjuvant, a stabilizer, an additive, or a combination thereof. In some embodiments, the seed formulation component is selected from one or more of polyvinylpyrrolidone (PVP), gum Arabic. and Xanthan gum. In some embodiments, the composition comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%. 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction; dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%, 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In another aspect, there are provided methods of protecting plant health, the method comprising contacting a plant seed to any composition provided herein: whereby germination rate, quality of germinated seed, or a combination thereof is improved as compared to an untreated plant seed. In some embodiments, the composition comprises a microorganism and at least one seed formulation component. In some embodiments, the microorganism is selected from Klebsiella, Bacillus licheniformis, Bacillus cereus, Exiguobacterium undeae, or a combination thereof. In some embodiments, the seed formulation component is an adjuvant, a stabilizer, an additive, or a combination thereof. In some embodiments, the seed formulation component is selected from one or more of polyvinylpyrrolidone (PVP), gum Arabic. and Xanthan gum. In some embodiments, the composition further comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the microorganism is selected from Klebsiella aerogenes, Bacillus licheniformis, Bacillus cereus. Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%. 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%, 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%. 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises a microorganism isolated from a plant growing in the high desert and at least one seed formulation component. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes, Bacillus licheniformis, Bacillus cereus, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the seed formulation component is an adjuvant, a stabilizer, an additive, or a combination thereof. In some embodiments, the seed formulation component is selected from one or more of polyvinylpyrrolidone (PVP), gum Arabic, and Xanthan gum. In some embodiments, the composition comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction; dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%. 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%. 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In a further aspect, provided herein are methods of increasing crop yield, the method comprising contacting a set of plant seeds to any composition provided herein: planting the set: growing plants from the planted set to harvest; and harvesting the plants or a portion thereof, wherein the crop yield is increased as compared to crop yield from an untreated set of plant seeds. In some embodiments, the composition comprises a microorganism and at least one seed formulation component. In some embodiments, the microorganism is selected from Klebsiella, Bacillus licheniformis. Bacillus cereus. Exiguobacterium undeae, or a combination thereof. In some embodiments, the seed formulation component is an adjuvant, a stabilizer, an additive, or a combination thereof. In some embodiments, the seed formulation component is selected from one or more of polyvinylpyrrolidone (PVP), gum Arabic. and Xanthan gum. In some embodiments, the composition further comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the microorganism is selected from Klebsiella aerogenes, Bacillus licheniformis, Bacillus cereus. Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%. 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%, 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%. 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises a microorganism isolated from a plant growing in the high desert and at least one seed formulation component. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes. Bacillus licheniformis. Bacillus cereus, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the seed formulation component is an adjuvant, a stabilizer, an additive, or a combination thereof. In some embodiments, the seed formulation component is selected from one or more of polyvinylpyrrolidone (PVP), gum Arabic, and Xanthan gum. In some embodiments, the composition comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction; dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%. 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%. 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In another aspect, provided herein are methods of promoting growth of a plant, the method comprising contacting seed of the plant to any composition provided herein: germinating the seed of the plant; and growing the resulting plant for a time period sufficient to develop leaves and roots, whereby biomass of the plant, root development of the plant, or a combination thereof is improved compared to a plant grown from an untreated seed. In some embodiments, root development comprises length of the roots, number of lateral roots, or a combination thereof. In some embodiments, the composition comprises a microorganism and at least one seed formulation component. In some embodiments, the microorganism is selected from Klebsiella, Bacillus licheniformis, Bacillus cereus, Exiguobacterium undeae, or a combination thereof. In some embodiments, the seed formulation component is an adjuvant, a stabilizer, an additive, or a combination thereof. In some embodiments, the seed formulation component is selected from one or more of polyvinylpyrrolidone (PVP), gum Arabic, and Xanthan gum. In some embodiments, the composition further comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the microorganism is selected from Klebsiella aerogenes. Bacillus licheniformis, Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%. 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%. 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 26. In some embodiments, the composition comprises a combination of Klebsiella aerogenes and Bacillus cereus in a 50:50 ratio (CFU/CFU). In some embodiments, the composition comprises a microorganism isolated from a plant growing in the high desert and at least one seed formulation component. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes, Bacillus licheniformis. Bacillus cereus, Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the seed formulation component is an adjuvant, a stabilizer, an additive, or a combination thereof. In some embodiments, the seed formulation component is selected from one or more of polyvinylpyrrolidone (PVP), gum Arabic. and Xanthan gum. In some embodiments, the composition comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the microorganism comprises a gene having at least about 70%, 80%, 90%, 95%, 99%, or 100% identity to a signature gene. In some embodiments, the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%. 99%, or 100% identity to a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus licheniformis and the Bacillus licheniformis comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26. In some embodiments, the composition comprises Bacillus licheniformis and the Bacillus licheniformis comprises a phosphate solubilization signature gene set forth in Table 29. In some embodiments, the composition comprises Bacillus licheniformis and the Bacillus licheniformis comprises a plant growth regulatory signature gene set forth in Table 46. In some embodiments, the composition comprises a combination of Klebsiella aerogenes and Bacillus licheniformis in a 50:50 ratio (CFU/CFU). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26. In some embodiments, the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%, 99%, or 100% identity to a phosphate solubilization signature gene set forth in Table 29. In some embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In another aspect, provided herein are methods of preparing a seed treatment, the method comprising growing a microorganism selected from a genus of Klebsiella, Bacillus, Exiguobacterium, or a combination thereof to at least 1×10⁸ CFU/g in a liquid media; and preparing a composition comprising a liquid media, the microorganism, at least one formulation component selected from polyvinylpyrrolidone (PVP), gum Arabic, and Xanthan gum. In some embodiments, the microorganism is Klebsiella aerogenes. Bacillus licheniformis, Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the method further comprises applying the seed treatment to a plant seed. In some embodiments, the liquid media comprises one or more of peptone, tryptone, or meat extract. In some embodiments, the microorganism is present at a concentration of greater than 1×10⁸ CFU/ml. In some embodiments, the microorganism is present at a concentration of from about 1×10⁴ CFU/ml to about 1×10¹⁰ CFU/ml. In some embodiments, the microorganism is present at a concentration of greater than 1×10⁸ CFU/ml for at least 6 months at 25° C. In some embodiments, the microorganism is present at a concentration of greater than 1×10⁸ CFU/ml for at least 6 months at a temperature from about 20° C. to about 35° C. In some embodiments, the microorganism is present at a concentration of from about 1×10⁴ CFU/ml to about 1×10¹⁰ CFU/ml for at least 6 months at 25° C. In some embodiments, the microorganism is present at a concentration of from about 1×10⁴ CFU/ml to about 1×10¹⁰ CFU/ml for at least 6 months at a temperature from about 20° C. to about 35° C.

In another aspect, provided herein are compositions comprising a microorganism and at least one soil or plant amendment component, wherein the microorganism is selected from Klebsiella, Bacillus cereus, Exiguobacterium undeae, or a combination thereof. In some embodiments, the soil or plant amendment comprises an adjuvant, a stabilizer, or an additive. In some embodiments, the soil or plant amendment comprises polyvinylpyrrolidone (PVP), gum Arabic, or Xanthan gum. In some embodiments, the composition further comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the microorganism is selected from Klebsiella aerogenes. Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, the strain CK3 has a DSMZ accession number DSM 34323. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum. In some embodiments, the composition confers plant growth regulatory activity. In some embodiments, the composition comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the composition comprises Bacillus cereus and the Bacillus cereus comprises a phosphate solubilization signature gene set forth in Table 29. In some embodiments, the composition comprises Bacillus cereus and the Bacillus cereus comprises a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In another aspect, provided herein are compositions comprising a microorganism isolated from a plant growing in the high desert and at least one soil or plant amendment component. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes. Bacillus cereus, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, the strain CK3 has a DSMZ accession number DSM 34323. In some embodiments, the soil or plant amendment comprises an adjuvant, a stabilizer, or an additive. In some embodiments, the soil or plant amendment component comprises polyvinylpyrrolidone (PVP), gum Arabic, or Xanthan gum. In some embodiments, the composition comprises one or more of peptone, tryptone, or meat extract. In some embodiments, the microorganism is present at a concentration of greater than about 1×10⁸ CFU/ml. In some embodiments, the microorganism is present at a concentration of from about 1×10⁴ CFU/ml to about 1×10¹⁰ CFU/ml. In some embodiments, the composition has a shelf life of at least about 6 months. In some embodiments, the composition is a liquid. In some embodiments, the composition confers anti-fungal activity. In some embodiments, the anti-fungal activity is against one or more of Macrophomina phaseolina, Fusarium sp., Fusarium tucumaniae Septoria sp., or Sclerotinia sclerotiorum. In some embodiments, the composition confers plant growth regulatory activity. In some embodiments, the composition comprises Klebsiella aerogenes and wherein the Klebsiella aerogenes comprises a nitrogen pathway signature comprises at least one of assimilatory nitrogen reduction; dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the composition comprises Klebsiella aerogenes and the Klebsiella aerogenes comprises a phosphate solubilization signature gene set forth in Table 29. In some embodiments, the composition comprises Klebsiella aerogenes and the Klebsiella aerogenes comprises a plant growth regulatory signature gene set forth in Table 35. In some embodiments, the composition comprises Klebsiella aerogenes and the Klebsiella aerogenes does not produce carbapenemase (KPC), metallo-beta-lactamases (MLB), or oxacillinase (Oxa). In some embodiments, the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the composition comprises Bacillus cereus and the Bacillus cereus comprises a phosphate solubilization signature gene set forth in Table 29. In some embodiments, the composition comprises Bacillus cereus and the Bacillus cereus comprises a plant growth regulatory signature gene set forth in Table 46. In some embodiments, the composition comprises a combination of Klebsiella aerogenes and Bacillus cereus in a 50:50 ratio (CFU/CFU). In some embodiments, the composition comprises water.

In another aspect, provided herein are plants grown with the composition comprising soil or plant amendments according to various embodiments herein.

In another aspect, provided herein are method of controlling fungal growth, the method comprising contacting a plant to the composition comprising soil or plant amendments according to various embodiments herein; and growing the plant under a condition capable of exposing the plant to a fungus, whereby the composition reduces growth of the fungus on or around the plant.

In another aspect, provided herein are methods of protecting plant health, the method comprising contacting a plant to the composition comprising soil or plant amendments according to various embodiments herein: whereby plant growth is improved as compared to an untreated plant.

In another aspect, provided herein are methods of increasing crop yield, the method comprising contacting a set of plants to the composition comprising soil or plant amendments according to various embodiments herein: growing plants from the set of plants to harvest; and harvesting the plants or a portion thereof, wherein the crop yield is increased as compared to crop yield from an untreated set of plants.

In another aspect, provided herein are methods of promoting growth of a plant, the method comprising contacting the plant to the composition comprising soil or plant amendments according to various embodiments herein: growing the plant for a time period sufficient to develop leaves and roots, whereby biomass of the plant, root development of the plant, or a combination thereof is improved compared to an untreated plant. In some embodiments, root development comprises length of the roots, number of lateral roots, or a combination thereof.

In another aspect, provided herein are methods of increasing fertility of a soil, the method comprising contacting a plurality of plants to the composition comprising soil or plant amendments according to various embodiments herein; and growing a plurality of plants in the soil, thereby increasing the fertility of the soil.

In another aspect, provided herein are methods of preparing a soil or plant amendment, the method comprising growing a microorganism selected from a genus of Klebsiella, Bacillus. Exiguobacterium, or a combination thereof to at least 1×10⁸ CFU/g in a liquid media; and preparing a composition comprising a liquid media, the microorganism, at least one formulation component selected from polyvinylpyrrolidone (PVP), gum Arabic, and Xanthan gum. In some embodiments, the microorganism is Klebsiella aerogenes. Bacillus licheniformis, Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the method further comprises applying the seed treatment to a plant seed. In some embodiments, the liquid media comprises one or more of peptone, tryptone, or meat extract. In some embodiments, the microorganism is present at a concentration of greater than 1×10⁸ CFU/ml. In some embodiments, the microorganism is present at a concentration of from about 1×10⁴ CFU/ml to about 1×10¹⁰ CFU/ml. In some embodiments, the microorganism is present at a concentration of greater than 1×10⁸ CFU/ml for at least 6 months at 25° C. In some embodiments, the microorganism is present at a concentration of greater than 1×10⁸ CFU/ml for at least 6 months at a temperature from about 20° C. to about 35° C. In some embodiments, the microorganism is present at a concentration of from about 1×10⁴ CFU/ml to about 1×10¹⁰ CFU/ml for at least 6 months at 25° C. In some embodiments, the microorganism is present at a concentration of greater than from about 1×10⁴ CFU/ml to about 1×10¹⁰ CFU/ml for at least 6 months at a temperature from about 20° C. to about 35° C.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows viability of CK1 in M1 medium after one month.

FIG. 2 shows a comparison of reducing sugars before and after heat by sterilization in M1 and M2 culture media.

FIG. 3 shows viability of CD1-M2 over time.

FIG. 4 shows KP, AN, and LB culture media for CD1 and CK2 growth.

FIG. 5 shows viability of CK1 and CK2 in AN, KB, and LB culture media for two months.

FIG. 6 shows crop pictures TUCUMAN (V5) site: A=CK1+CK2 6 m, B=Control vs CK1 6ML, C=Control vs CK1+CK2 5 ml.

FIG. 7 shows crop pictures SAN JERONIMO NORTE (V5) site: A=CK1+CK2 6 m, B=Control vs CK1 6ML, C=Control vs CK1+CK2 5 ml.

FIG. 8 shows phosphate solubilization genes in CK1.

FIG. 9 shows phosphate solubilization genes in CK2.

FIG. 10 shows CK1 pathogenicity.

FIG. 11 shows biofungicide activity against Sclerotinia sclerotium.

FIG. 12 shows biofungicide activity against Fusarium.

FIG. 13 shows treated soybean seeds were planted to analyze the effects of the products against F. tucumaniae.

FIG. 14 shows results of a greenhouse trial to evaluate the effectiveness of seed treatments against F. tucumaniae in soybean (DM5958) at 20 days after planting.

FIG. 15 shows treated soybean seeds (M6410 IPRO) were planted to analyze the effects of the products against M. phaseolina.

FIG. 16 shows results of seed treatment assay against M. phaseolina in soybean (M6410) 10 days after planting.

FIG. 17 shows an average nucleotide identity heatmap with ANI values. The closest strain to B. cereus CK2 is B. cereus A1 with an ANI value of 0.99.

FIG. 18 shows a phylogenetic tree reconstruction based on ANI values distance matrix using the neighbor joining method with PHYLIP. Strain CK2 is placed in the B. cereus species.

FIG. 19 shows imaging of roots with Confocal Laser Scanning Microscope (left panel) control and (right panel) CK1-GFP.

FIG. 20 shows imaging of leaves with Confocal Laser Scanning Microscope (left panel) control and (right panel) CK1-GFP.

FIG. 21 shows imaging of stems with Confocal Laser Scanning Microscope (left panel) control and (right panel) CK1-GFP.

FIG. 22 shows ion exchange chromatography in DE52 of the EPS sample. In a dotted line, the concentration of NaCl in the elution is plotted.

FIG. 23 shows a spectrum 1H NMR of purified EPS. Above, the spectrum between 5.6 and 3 ppm is illustrated. Below, the resonances of the identified anomeric protons (A-H) in the zone between 5.6 and 4.8 ppm are indicated.

FIG. 24 shows ¹H-¹³C-HSQC spectra superimposed on hydrolyzed EPS (blue) and D-Mannose (alpha and β) (light blue), D-Glucose (alpha and B) (green), D-Galactose (alpha and B) (red) and D-Glucosamine (alpha and B) (brown). Peaks in the spectrum of hydrolyzed EPS that do not overlap with standards belong to remaining non-hydrolyzed EPS molecules.

FIG. 25 shows HPAEC-PAD analysis of hydrolyzed EPS. The circumvention times of the different monosaccharides identified are indicated by arrows.

FIG. 26 shows HPAEC-PAD analysis of hydrolyzed EPS.

FIG. 27 shows a spectrum ¹H-¹³C-HSQC of MRI of purified EPS. The region of the spectrum corresponding to anomeric protons and carbons is plotted.

FIG. 28 shows ¹H-1H-TOCSY spectrum of purified EPS NMR. The spectrum region is plotted between 5.6 and 4.6 ppm. The correlations between the anomeric ¹H and ¹H2 of each residue are indicated.

FIG. 29 shows ¹H-1H-NOESY MRI spectrum of purified EPS. The spectrum region is plotted between 5.6 and 4.6 ppm. Correlations between anomeric ¹H and ¹HX between residues are indicated.

FIG. 30 shows five panels of mass spectra of monosaccharides derived from purified EPS. Panels 1, 2, 3, 4 and 5 correspond to species 1, 2, 3, 4 and 5 identified in Table 48, respectively.

### DETAILED DESCRIPTION

Providing improved crop yields in an ever increasing hostile climate will be essential to a growing worldwide population. Provided herein are certain compositions comprising bacterial strains, including seed treatment compositions, that increase the fitness of the plants from treated seeds or soils. In some cases, these bacterial compositions allow crop growth in poor soils that have been degraded by drought, high salinity, and other effects of climate change.

### Compositions

Accordingly, provided herein are compositions comprising mixtures of bacterial strains and methods of use in crop cultivation. In one aspect, there are provided compositions comprising a microorganism and at least one seed formulation component. In some embodiments, the microorganism is selected from Klebsiella, Bacillus licheniformis, Bacillus cereus, Exiguobacterium undeae, or a combination thereof. In some embodiments, the microorganism is selected from Klebsiella aerogenes, Bacillus licheniformis, Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323. In some embodiments, the composition is a soil amendment. In some embodiments, the composition is a plant amendment. In some embodiments, the composition comprises water.

In another aspect, there are provided compositions comprising a microorganism isolated from a plant growing in a harsh environment, such as the high desert, and at least one seed formulation component. As used herein. “harsh environment” refers to a climate with suboptimal rainfall, extremes of heat and cold, and/or high elevation compared to a temperate climate. As used herein, the “high desert” refers to a desert region that is located at a high elevation, such as over 3000 feet (900 meters). In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes. Bacillus cereus, Bacillus licheniformis, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments the Exiguobacterium undeae is a strain CK3 or a derivative thereof. In some embodiments, wherein the strain CK3 has a DSMZ accession number DSM 34323. In some embodiments, the composition is a soil amendment. In some embodiments, the composition is a plant amendment. In some embodiments, the composition comprises water.

In embodiments, the seed formulation component comprises any suitable substance for stabilizing the seeds and/or the bacterial strains. In some embodiments, the seed formulation component is a polymer or a detergent. In some embodiments, the seed formulation component is an adjuvant, a stabilizer, an additive, or a combination thereof. For example, in some embodiments, the seed formulation component is selected from one or more of polyvinylpyrrolidone (PVP), gum Arabic. and Xanthan gum. In some embodiments, the composition comprises a nutrient source. For example, in some embodiments, the composition comprises one or more of peptone, tryptone, or meat extract. In some embodiments, the microorganism is present at a concentration of greater than about 1×10⁴, about 1×10⁵, about 1×10⁶, about 1×10⁷, about 1×10⁸ about 1×10⁹, or about 1×10¹⁰ CFU/ml. In some embodiments, the composition has a shelf life of at least about 2 months, about 3 months, about 4 months, about 5 months, about 6 months, about 7 months, about 8 months, about 9 months, about 10 months, about 11 months, or about 12 months. In some embodiments, the composition is a liquid. In some embodiments, the composition is a gel or suspension. In some embodiments, the composition is a soil amendment. In some embodiments, the composition is a plant amendment. In some embodiments, the composition comprises water.

In embodiments, the composition confers anti-fungal activity. In some embodiments, the anti-fungal activity is against one or more of Macrophomina phaseolina, Fusarium sp., Fusarium tucumaniae, Septoria sp., or Sclerotinia sclerotiorum.

In embodiments, the composition confers plant growth regulatory activity. In some embodiments, the microorganism comprises a gene having at least about 70%, 80%, 90%, 95%, 99%, or 100% identity to a signature gene.

In embodiments, the seed treatment comprises Klebsiella aerogenes. In some embodiments, the Klebsiella aerogenes comprises a nitrogen pathway signature. In some embodiments, the nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25.

In embodiments, the composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29.

In embodiments, the composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35.

In embodiments, the composition comprises Klebsiella aerogenes and the Klebsiella aerogenes does not produce carbapenemase (KPC), metallo-beta-lactamases (MLB), or oxacillinase (Oxa).

In embodiments, the composition comprises Bacillus licheniformis. In some embodiments, the Bacillus licheniformis comprises a nitrogen pathway signature. In some embodiments, the nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26.

In embodiments the composition comprises Bacillus licheniformis and the Bacillus licheniformis comprises a phosphate solubilization signature gene set forth in Table 29.

In embodiments, the composition comprises Bacillus licheniformis and the Bacillus licheniformis comprises a plant growth regulatory signature gene set forth in Table 46.

In embodiments, compositions herein comprise a combination of Klebsiella aerogenes and Bacillus licheniformis. In some embodiments, the composition comprises a combination of Klebsiella aerogenes and Bacillus licheniformis in a 10:90, 20:80, 30:70, 40:60, 50:50, 60:40, 70:30, 80:20, or 90:10 ratio (CFU/CFU). In some embodiments, the composition comprises a combination of Klebsiella aerogenes and Bacillus licheniformis in a 50:50 ratio (CFU/CFU). In some embodiments, the composition comprises water.

In embodiments, the composition comprises Bacillus cereus. In some embodiments, the Bacillus cereus comprises a nitrogen pathway signature. In some embodiments, the nitrogen pathway signature comprises at least one of assimilatory nitrogen reduction; dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26.

In embodiments the composition comprises Bacillus cereus and the Bacillus cereus comprises a phosphate solubilization signature gene set forth in Table 29. In some embodiments, the Bacillus cereus comprises a gene having at least about 70%, 80%, 90%, 95%, 99%, or 100% identity to a phosphate solubilization signature gene set forth in Table 29.

In embodiments, the composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46.

In embodiments, compositions herein comprise a combination of Klebsiella aerogenes and Bacillus cereus. In some embodiments, the composition comprises a combination of Klebsiella aerogenes and Bacillus cereus in a 10:90, 20:80, 30:70, 40:60, 50:50, 60:40, 70:30, 80:20, or 90:10 ratio (CFU/CFU). In some embodiments, the composition comprises a combination of Klebsiella aerogenes and Bacillus cereus in a 50:50 ratio (CFU/CFU). In some embodiments, the composition is a soil amendment. In some embodiments, the composition is a plant amendment. In some embodiments, the composition comprises water.

Treated Seeds and Plants Grown from Treated Seeds

In another aspect, provided herein are treated seeds comprising a plant seed and a seed treatment composition disclosed herein. In some embodiments, the seed treatment composition comprises a microorganism selected from Klebsiella, Bacillus licheniformis, Bacillus cereus, Exiguobacterium undeae, or a combination thereof. In some embodiments, the microorganism is selected from Klebsiella aerogenes. Bacillus licheniformis, Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323.

In another aspect, there are provided treated seeds comprising a plant seed and a seed treatment composition comprising a microorganism isolated from a plant growing in a harsh environment, such as the high desert. In some embodiments, the microorganism is isolated from a plant growing in Puna de Atacama and at least one seed formulation component. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes, Bacillus licheniformis, Bacillus cereus, Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323.

In a further aspect, there are provided plants grown from treated seeds provided herein.

In embodiments, the seed treatment composition comprises any suitable substance for stabilizing the seeds and/or the bacterial strains. In some embodiments, the seed formulation component is a polymer or a detergent. In some embodiments, the seed formulation component is an adjuvant, a stabilizer, an additive, or a combination thereof. For example, in some embodiments, the seed formulation component is selected from one or more of polyvinylpyrrolidone (PVP), gum Arabic, and Xanthan gum. In some embodiments, the composition comprises a nutrient source. For example, in some embodiments, the composition comprises one or more of peptone, tryptone, or meat extract. In some embodiments, the microorganism is present at a concentration of greater than about 1×10⁴, about 1×10⁵, about 1×10⁶, about 1×10⁷, about 1×10⁸ about 1×10⁹, or about 1×10¹⁰ CFU/ml. In some embodiments, the composition has a shelf life of at least about 2 months, about 3 months, about 4 months, about 5 months, about 6 months, about 7 months, about 8 months, about 9 months, about 10 months, about 11 months, or about 12 months. In some embodiments, the composition is a liquid. In some embodiments, the composition is a gel or suspension.

In embodiments, the seed treatment composition confers anti-fungal activity. In some embodiments, the anti-fungal activity is against one or more of Macrophomina phaseolina, Fusarium sp., Fusarium tucumaniae, Septoria sp., or Sclerotinia sclerotiorum.

In embodiments, the seed treatment composition confers plant growth regulatory activity. In some embodiments, the microorganism comprises a gene having at least about 70%, 80%, 90%, 95%. 99%, or 100% identity to a signature gene.

In embodiments, the seed treatment comprises Klebsiella aerogenes. In some embodiments, the Klebsiella aerogenes comprises a nitrogen pathway signature. In some embodiments, the nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25.

In embodiments, the seed treatment composition comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%. 99%, or 100% identity to a phosphate solubilization signature gene set forth in Table 29.

In embodiments, the seed treatment composition comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%. 99%, or 100% identity to a plant growth regulatory signature gene set forth in Table 35.

In embodiments, the seed treatment composition comprises Klebsiella aerogenes and the Klebsiella aerogenes does not produce carbapenemase (KPC), metallo-beta-lactamases (MLB), or oxacillinase (Oxa).

In embodiments, the composition comprises Bacillus licheniformis. In some embodiments, the Bacillus licheniformis comprises a nitrogen pathway signature. In some embodiments, the nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26.

In embodiments the seed treatment composition comprises Bacillus licheniformis and the Bacillus licheniformis comprises a phosphate solubilization signature gene set forth in Table 29.

In embodiments, the seed treatment composition comprises Bacillus licheniformis and the Bacillus licheniformis comprises a plant growth regulatory signature gene set forth in Table 46.

In embodiments, the seed treatment composition comprises a combination of Klebsiella aerogenes and Bacillus licheniformis. In some embodiments, the composition comprises a combination of Klebsiella aerogenes and Bacillus licheniformis in a 10:90, 20:80, 30:70, 40:60, 50:50, 60:40, 70:30, 80:20, or 90:10 ratio (CFU/CFU). In some embodiments, the composition comprises a combination of Klebsiella aerogenes and Bacillus licheniformis in a 50:50 ratio (CFU/CFU).

In embodiments, the composition comprises Bacillus cereus. In some embodiments, the Bacillus cereus comprises a nitrogen pathway signature. In some embodiments, the nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%, 99%, or 100% identity to a nitrogen pathway signature gene set forth in Table 26.

In embodiments the seed treatment composition comprises Bacillus cereus and the Bacillus cereus comprises a phosphate solubilization signature gene set forth in Table 29. In some embodiments the Bacillus cereus comprises a gene having at least about 70%, 80%, 90%, 95%, 99%, or 100% identity to a phosphate solubilization signature gene set forth in Table 29.

In embodiments, the seed treatment composition comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46.

In embodiments, the seed treatment composition comprises a combination of Klebsiella aerogenes and Bacillus cereus. In some embodiments, the composition comprises a combination of Klebsiella aerogenes and Bacillus cereus in a 10:90, 20:80, 30:70, 40:60, 50:50, 60:40, 70:30, 80:20, or 90:10 ratio (CFU/CFU). In some embodiments, the composition comprises a combination of Klebsiella aerogenes and Bacillus cereus in a 50:50 ratio (CFU/CFU).

### Soil or Plant Amendments

In another aspect, provided herein are soil or plant amendments comprising a composition disclosed herein. In some embodiments, the composition comprises a microorganism selected from Klebsiella, Bacillus licheniformis, Bacillus cereus, Exiguobacterium undeae, or a combination thereof. In some embodiments, the microorganism is selected from Klebsiella aerogenes. Bacillus licheniformis. Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae, is a strain CK3 or a derivative thereof. In some embodiments, wherein the strain CK3 has a DSMZ accession number DSM 34323. In some embodiments, the composition comprises water.

In another aspect, there are provided soil or plant amendments comprising a microorganism isolated from a plant growing in a harsh environment, such as the high desert. In some embodiments, the microorganism is isolated from a plant growing in Puna de Atacama and at least one seed formulation component. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes, Bacillus licheniformis. Bacillus cereus. Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323. In some embodiments, the composition comprises water.

In a further aspect, there are provided plants grown using soil or plant amendments provided herein.

In embodiments, the soil or plant amendment comprises any suitable substance for stabilizing the bacterial strains. In some embodiments, the soil or plant amendment comprises a nutrient source. For example, in some embodiments, the soil or plant amendment comprises one or more of peptone, tryptone, or meat extract. In some embodiments, the microorganism is present at a concentration of greater than about 1×10⁴, about 1×10⁵, about 1×10⁶, about 1×10⁷, about 1×10⁸ about 1×10⁹, or about 1×10¹⁰ CFU/ml. In some embodiments, the composition has a shelf life of at least about 2 months, about 3 months, about 4 months, about 5 months, about 6 months, about 7 months, about 8 months, about 9 months, about 10 months, about 11 months, or about 12 months. In some embodiments, the composition is a liquid. In some embodiments, the composition is a gel or suspension. In some embodiments, the composition comprises water.

In embodiments, the soil or plant amendment confers anti-fungal activity. In some embodiments, the anti-fungal activity is against one or more of Macrophomina phaseolina, Fusarium sp., Fusarium tucumaniae, Septoria sp., or Sclerotinia sclerotiorum.

In embodiments, the seed treatment composition confers plant growth regulatory activity. In some embodiments, the microorganism comprises a gene having at least about 70%, 80%, 90%, 95%, 99%, or 100% identity to a signature gene.

In embodiments, the soil or plant amendment comprises Klebsiella aerogenes. In some embodiments, the Klebsiella aerogenes comprises a nitrogen pathway signature. In some embodiments, the nitrogen pathway signature comprises at least one of assimilatory nitrogen reduction: dissimilatory nitrate reduction; and the absence of all or a portion of nitrogen fixation, nitrification, and denitrification. In some embodiments, the composition comprises Klebsiella aerogenes and the Klebsiella aerogenes comprises a nitrogen pathway signature gene set forth in Table 25. In some embodiments, the Klebsiella aerogenes comprises a gene having at least about 70%, 80%, 90%, 95%, 99%, or 100% identity to a nitrogen pathway signature gene set forth in Table 25.

In embodiments, the soil or plant amendment comprises Klebsiella aerogenes and the Klebsiella aerogenes comprises a phosphate solubilization signature gene set forth in Table 29. In some embodiments, the Klebsiella aerogenes comprises a gene having at least about 70%, 80%, 90%, 95%, 99%, or 100% identity to a phosphate solubilization signature gene set forth in Table 29.

In embodiments, the soil or plant amendment comprises Klebsiella aerogenes and the Klebsiella aerogenes comprises a plant growth regulatory signature gene set forth in Table 35. In some embodiments, the Klebsiella aerogenes comprises a gene having at least about 70%, 80%, 90%, 95%, 99%, or 100% identity to a plant growth regulatory signature gene set forth in Table 35.

In embodiments, the soil or plant amendment comprises Klebsiella aerogenes and the Klebsiella aerogenes does not produce carbapenemase (KPC), metallo-beta-lactamases (MLB), or oxacillinase (Oxa).

In embodiments, the composition comprises Bacillus licheniformis. In some embodiments, the Bacillus licheniformis comprises a nitrogen pathway signature. In some embodiments, the nitrogen pathway signature comprises at least one of assimilatory nitrogen reduction: dissimilatory nitrate reduction; and the absence of all or a portion of nitrogen fixation, nitrification, and denitrification. In some embodiments, the Bacillus licheniformis comprises a nitrogen pathway signature gene set forth in Table 26.

In embodiments the soil or plant amendment comprises Bacillus licheniformis and the Bacillus licheniformis comprises a phosphate solubilization signature gene set forth in Table 29.

In embodiments, the soil or plant amendment comprises Bacillus licheniformis and the Bacillus licheniformis comprises a plant growth regulatory signature gene set forth in Table 46.

In embodiments, the soil or plant amendment comprises a combination of Klebsiella aerogenes and Bacillus licheniformis. In some embodiments, the composition comprises a combination of Klebsiella aerogenes and Bacillus licheniformis in a 10:90, 20:80, 30:70, 40:60, 50:50, 60:40, 70:30, 80:20, or 90:10 ratio (CFU/CFU). In some embodiments, the composition comprises a combination of Klebsiella aerogenes and Bacillus licheniformis in a 50:50 ratio (CFU/CFU).

In embodiments, the soil or plant amendment comprises Bacillus cereus. In some embodiments, the Bacillus cereus comprises a nitrogen pathway signature. In some embodiments, the nitrogen pathway signature comprises at least one of assimilatory nitrogen reduction: dissimilatory nitrate reduction; and the absence of all or a portion of nitrogen fixation, nitrification, and denitrification. In some embodiments, the Bacillus cereus comprises a nitrogen pathway signature gene set forth in Table 26. In some embodiments, the Bacillus cereus comprises a gene having at least about 70%, 80%, 90%, 95%. 99%, or 100% identity to a nitrogen pathway signature gene set forth in Table 26.

In embodiments the soil or plant amendment comprises Bacillus cereus and the Bacillus cereus comprises a phosphate solubilization signature gene set forth in Table 29. In some embodiments, the Bacillus cereus comprises a gene having at least about 70%, 80%, 90%, 95%, 99%, or 100% identity to a phosphate solubilization signature gene set forth in Table 29.

In embodiments, the soil or plant amendment comprises Bacillus cereus and the Bacillus cereus comprises a plant growth regulatory signature gene set forth in Table 46. In some embodiments, the Bacillus cereus comprises a gene having at least about 70%, 80%, 90%, 95%, 99%, or 100% identity to a plant growth regulatory signature gene set forth in Table 46.

In embodiments, the soil or plant amendment comprises a combination of Klebsiella aerogenes and Bacillus cereus. In some embodiments, the composition comprises a combination of Klebsiella aerogenes and Bacillus cereus in a 10:90, 20:80, 30:70, 40:60, 50:50, 60:40, 70:30, 80:20, or 90:10 ratio (CFU/CFU). In some embodiments, the composition comprises a combination of Klebsiella aerogenes and Bacillus cereus in a 50:50 ratio (CFU/CFU). In some embodiments, the composition comprises water.

### Methods of Controlling Fungal Growth and Protecting Plant Health

In another aspect, provided herein are methods of controlling fungal growth. In some embodiments, the method comprises contacting a plant seed to any composition provided herein. In some embodiments, the method comprises germinating the plant seed under a condition capable of exposing the plant seed to a fungus. In some embodiments, the seed treatment reduces growth of the fungus on or around the plant seed. In some embodiments, the fungus comprises one or more of Macrophomina phaseolina, Fusarium sp., Fusarium tucumaniae, Septoria sp., or Sclerotinia sclerotiorum.

In a further aspect, provided herein are methods of protecting plant health. In some embodiments, the method comprises contacting a plant seed to any composition provided herein. In some embodiments, germination rate, quality of germinated seed, or a combination thereof is improved as compared to an untreated plant seed. In some embodiments, the composition is a soil amendment. In some embodiments, the composition is a plant amendment.

### Methods of Increasing Crop Yield and Plant Growth

In another aspect, provided herein are methods of increasing crop yield. In some embodiments, the method comprising contacting a set of plant seeds to any composition provided herein. In some embodiments, the method comprises planting the set. In some embodiments, the method comprises growing plants from the planted set to harvest. In some embodiments, the method comprises harvesting the plants or a portion thereof. In some embodiments, the crop yield is increased as compared to crop yield from an untreated set of plant seeds. In some embodiments, the crop yield is increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 120%, about 140%, about 160%, about 180%, about 200% or more compared to crop yield from an untreated set of plant seeds.

In another aspect, provided herein are methods of promoting growth of a plant. In some embodiments, the method comprises contacting seed of the plant to any composition provided herein. In some embodiments, the method comprises germinating the seed of the plant. In some embodiments, the method comprises growing the resulting plant for a time period sufficient to develop leaves and roots. In some embodiments, biomass of the plant, root development of the plant, or a combination thereof is improved compared to a plant grown from an untreated seed. In some embodiments, root development comprises length of the roots, number of lateral roots, or a combination thereof. In some embodiments, the biomass of the plant, root development of the plant, or a combination thereof is increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 120%, about 140%, about 160%, about 180%, about 200% or more compared to the biomass of the plant, root development of the plant, or a combination thereof from an untreated set of plant seeds. In some embodiments, the composition is a soil amendment. In some embodiments, the composition is a plant amendment.

### Methods of Preparing Seed Treatments

In an aspect, provided herein are methods of preparing a seed treatment. In some embodiments, the method comprising growing a microorganism selected from a genus of Klebsiella, Bacillus, Exiguobacterium, or a combination thereof to at least 1×10⁸ CFU/g in a liquid media. In some embodiments, the method comprises preparing a composition comprising a liquid media, the microorganism, at least one formulation component. In some embodiments, the formulation component is a polymer or a detergent. In some embodiments, the seed formulation component is an adjuvant, a stabilizer, an additive, or a combination thereof. In some embodiments, the formulation component is selected from one or more of polyvinylpyrrolidone (PVP), gum Arabic, and Xanthan gum. In some embodiments, the microorganism is Klebsiella aerogenes, Bacillus licheniformis. Bacillus cereus, Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323.

In an aspect, provided herein are methods of preparing a seed treatment. In some embodiments, the method comprises growing a microorganism isolated from a plant growing in a harsh environment, such as the high desert. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes. Bacillus licheniformis. Bacillus cereus, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323.

In aspects, the method further comprises comprising applying the seed treatment to a plant seed.

In embodiments, the seed formulation component comprises any suitable substance for stabilizing the seeds and/or the bacterial strains. In some embodiments, the seed formulation component is a polymer or a detergent. In some embodiments, the seed formulation component is an adjuvant, a stabilizer, an additive, or a combination thereof. For example, in some embodiments, the seed formulation component is selected from one or more of polyvinylpyrrolidone (PVP), gum Arabic, and Xanthan gum. In some embodiments, the composition comprises a nutrient source. For example, in some embodiments, the composition comprises one or more of peptone, tryptone, or meat extract. In some embodiments, the microorganism is present at a concentration of greater than about 1×10⁴, about 1×10⁵, about 1×10⁶, about 1×10⁷, about 1×10⁸ about 1×10⁹, or about 1×10¹⁰ CFU/ml. In some embodiments, the composition has a shelf life of at least about 2 months, about 3 months, about 4 months, about 5 months, about 6 months, about 7 months, about 8 months, about 9 months, about 10 months, about 11 months, or about 12 months. In some embodiments, the microorganism is present at a concentration of greater than 1×10⁸ CFU/ml for at least 6 months at 25° C. In some embodiments, the microorganism is present at a concentration of greater than 1×10⁸ CFU/ml for at least 6 months at a temperature from about 20° C. to about 35° C. In some embodiments, the microorganism is present at a concentration of from about 1×10⁴ CFU/ml to about 1×10¹⁰ CFU/ml for at least 6 months at 25° C. In some embodiments, the microorganism is present at a concentration of from about 1×10⁴ CFU/ml to about 1×10¹⁰ CFU/ml for at least 6 months at a temperature from about 20° C. to about 35° C. In some embodiments, the composition is a liquid. In some embodiments, the composition is a gel or suspension.

### Methods of Preparing Soil or Plant Amendments

In an aspect, provided herein are methods of preparing a soil or plant amendment. In some embodiments, the method comprising growing a microorganism selected from a genus of Klebsiella. Bacillus. Exiguobacterium, or a combination thereof to at least 1×10⁸ CFU/g in a liquid media. In some embodiments, the method comprises preparing a composition comprising a liquid media, the microorganism, at least one formulation component. In some embodiments, the formulation component is a polymer or a detergent. In some embodiments, the formulation component is an adjuvant, a stabilizer, an additive, or a combination thereof. In some embodiments, the formulation component is selected from one or more of polyvinylpyrrolidone (PVP), gum Arabic, and Xanthan gum. In some embodiments, the microorganism is Klebsiella aerogenes, Bacillus licheniformis, Bacillus cereus. Exiguobacterium undeae, or a combination thereof. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323.

In an aspect, provided herein are methods of preparing a soil or plant amendment. In some embodiments, the method comprises growing a microorganism isolated from a plant growing in a harsh environment, such as the high desert. In some embodiments, the microorganism is isolated from a plant growing in Puna de Atacama. In some embodiments, the microorganism is isolated from a rhizosphere of the plant. In some embodiments, the microorganism is isolated from a soil or sediments of the plant. In some embodiments, the bacterium is of the genus Klebsiella, Bacillus, or Exiguobacterium. In some embodiments, the bacterium is Klebsiella aerogenes. Bacillus licheniformis, Bacillus cereus, or Exiguobacterium undeae. In some embodiments, the Klebsiella aerogenes is a strain CK1 or a derivative thereof. In some embodiments, the strain CK1 has a DSMZ accession number DSM 34332. In some embodiments, the Bacillus licheniformis is a strain CK2 or a derivative thereof. In some embodiments, the Bacillus cereus is a strain CK2 or a derivative thereof. In some embodiments, the Bacillus cereus strain CK2 has a DSMZ accession number DSM 34322. In some embodiments, the Exiguobacterium undeae is a strain CK3 or a derivative thereof. In some embodiments, wherein the strain CK3 has a DSMZ accession number DSM 34323.

In aspects, the method further comprises comprising applying the soil or plant amendment to a plant or a plant seed.

In embodiments, the soil or plant amendment comprises any suitable substance for stabilizing the bacterial strains. In some embodiments, the seed formulation component is a polymer or a detergent. In some embodiments, the soil or plant amendment comprises an adjuvant, a stabilizer, or an additive. For example, in some embodiments, the soil or plant amendment comprises polyvinylpyrrolidone (PVP), gum Arabic, or Xanthan gum. In some embodiments, the soil or plant amendment comprises a nutrient source. For example, in some embodiments, the soil or plant amendment comprises one or more of peptone, tryptone, or meat extract. In some embodiments, the microorganism is present at a concentration of greater than about 1×10⁴, about 1×10⁵, about 1×10⁶, about 1×10⁷, about 1×10⁸ about 1×10⁹, or about 1×10¹⁰ CFU/ml. In some embodiments, the composition has a shelf life of at least about 2 months, about 3 months, about 4 months, about 5 months, about 6 months, about 7 months, about 8 months, about 9 months, about 10 months, about 11 months, or about 12 months. In some embodiments, the microorganism is present at a concentration of greater than 1×10⁸ CFU/ml for at least 6 months at 25° C. In some embodiments, the microorganism is present at a concentration of greater than 1×10⁸ CFU/ml for at least 6 months at a temperature from about 20° C. to about 35° C. In some embodiments, the microorganism is present at a concentration of from about 1×10⁴ CFU/ml to about 1×10¹⁰ CFU/ml for at least 6 months at 25° C. In some embodiments, the microorganism is present at a concentration of from about 1×10⁴ CFU/ml to about 1×10¹⁰ CFU/ml for at least 6 months at a temperature from about 20° C. to about 35° C. In some embodiments, the composition is a liquid. In some embodiments, the composition is a gel or suspension.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Media Evaluation and Seed Treatment Formulation

Bacterial strains CK1 (Klebsiella aerogenes). CK2 (Bacillus cereus) were isolated from the rhizosphere of plants growing next to Volcán Galán, provincia de Catamarca. Argentina.

CK1 and CK2 were evaluated for their ability to form viable colonies after growth in a variety of culture media.

For all assays the bacterial cultures were grown at 30° C., and 230 rpm. After 10 hours of culture, CFU/mL and pH of each culture was measured. The components of each culture media were listed in Table 1, all media sterilizations were carried out with autoclave, at 121° C. for 15 minutes.

In all cases, first the selection of a culture medium was based on achieving a high CFU/ml count during cultivation, then its shelf life was controlled by addition of the adjuvant (formulated). Xanthan gum (Phernhofen) was used as an adjuvant, and it was added to culture broth when shelf life was evaluated in 0.5% final concentration.

For check viability the samples were kept at room temperature, protected from light.

TABLE 1
Components and concentrations used for different culture media tested

| Culture | Previous | (NH₄)₂H | Yeast | Soy peptone | Glucose | | |
|---|---|---|---|---|---|---|---|
| Media | denomination | SO₄ g/L | Ext. g/L | g/L | g/L | K₂HPO₄ | KH₂PO₄ |
| M1 | ECO | 5 | 3 | — | 3 | — | — |
| M2 | M7 | 3 | 3 | — | 3 | — | — |
| M3 | Medio 1 | | | 5 | 15 | 4 | 6 |
| M4 | Medio 2 | 2 | 1.5 | — | — | 0.125 | — |
| M5 | Medio 4 | — | 5 | 10 | 2 | — | — |
| M6 | Medio 5 | — | 0.5 | 10 | — | — | — |
| M7 | 2 ′ (prima) | 2 | 3 | — | — | 0.125 | — |
| M8 | 2A | 2 | — | 3 | — | 0.125 | — |
| AN | | — | — | — | — | — | — |
| LB | | — | 5 | — | — | — | — |
| KB | | — | — | — | — | — | — |

| Culture | | | | | | | Trisodium |
|---|---|---|---|---|---|---|---|
| Media | (NH₄)₂SO₄ | MgSO₄•7H₂0 | KCl | MnSO₄•7H₂0 | NaCl | FeSO₄•7H₂O | citrate g/L |
| M1 | — | — | — | — | — | — | — |
| M2 | — | — | — | — | — | — | — |
| M3 | 2 | 0.2 | — | — | — | — | 1 |
| M4 | — | 0.2 | — | — | — | — | 7.5 |
| M5 | — | — | — | — | — | — | — |
| M6 | 0.5 | 0.1 | 0.2 | 0.004 | 0.2 | 0.002 | — |
| M7 | — | 0.2 | — | — | — | — | — |
| M8 | — | 0.2 | — | — | — | — | — |
| AN | — | — | — | — | — | — | — |
| LB | — | — | — | — | — | — | — |
| KB | — | — | — | — | — | — | — |

| Culture | | meat | | Pluri- | Meat | Sodium | | |
|---|---|---|---|---|---|---|---|---|
| Media | Sucrose g/L | extract | Tryptone | peptone | peptone | Chloride | K₂PO₄ | MgSO₄ |
| M1 | — | — | — | — | — | — | — | — |
| M2 | — | — | — | — | — | — | — | — |
| M3 | — | — | — | — | — | — | — | — |
| M4 | — | — | — | — | — | — | — | — |
| M5 | — | — | — | — | — | — | — | — |
| M6 | — | — | — | — | — | — | — | — |
| M7 | 7.5 | — | — | — | — | — | — | — |
| M8 | 7.5 | — | — | — | — | — | — | — |
| AN | — | 3 | — | 5 | — | 8 | — | — |
| LB | — | — | 10 | — | — | 10 | — | — |
| KB | — | — | 10 | — | 10 | — | 1.5 | 1.5 |

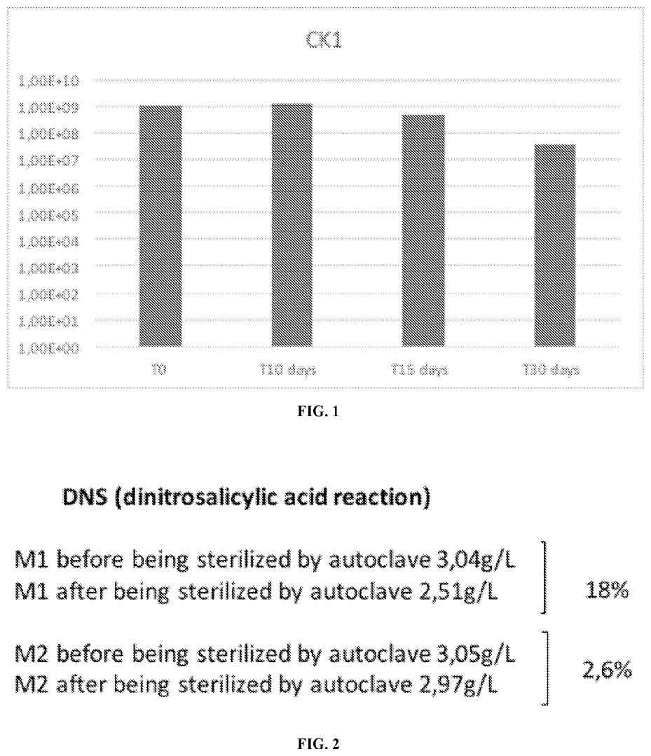

CK1 and CK2 were routinely cultivated in M1 medium. Even when the CFU/ml value for both strains was adequate (Table 2), this culture medium was discarded because the development of the formulation required a culture medium that sustains the viability of the cells over time. As shown in FIG. 1, when CK1 strain viability was tested in M1, a significant loss of viability after 15 days of storage was observed.

TABLE 2
Parameters evaluated for CK1 and CK2 growth in M1 medium

| | M1 | | |
|---|---|---|---|
| Strain | pH (after culture) | OD600 nm | CFU/ml |
| Klebsiella aerogenes CK1 | 4.80 | 1.24 | 2.65E+09 |
| Bacillus cereus CK2 | 5.10 | 1.25 | 1.53E+08 |

On the other hand, M1 medium was seen that after sterilization the Maillard reaction was produced in the culture medium used. The Maillard reaction is a non-desirable chemical reaction that occurs in the presence of heat between carbonyl compounds, especially reducing sugars like glucose, with compounds which possess a free amino group, such as amino acids, amines, and protein. This generates a brownish color and a decrease of reducing sugars, which should be available for consumption by the bacteria for their growth.

The reducing sugars were determined by DNS technique and the values obtained before and after heat are shown in FIG. 2. The reducing sugar in M1 culture medium decreased an 18%, from 3.04 g/L to 2.51 g/L after being exposed to heat. In contrast, M2 medium, containing sucrose (non-reducing sugar) instead of glucose as a carbon source, emerges as a viable alternative to M1 medium, because of showed a sugar loss only of 2.6% after the sterilization process.

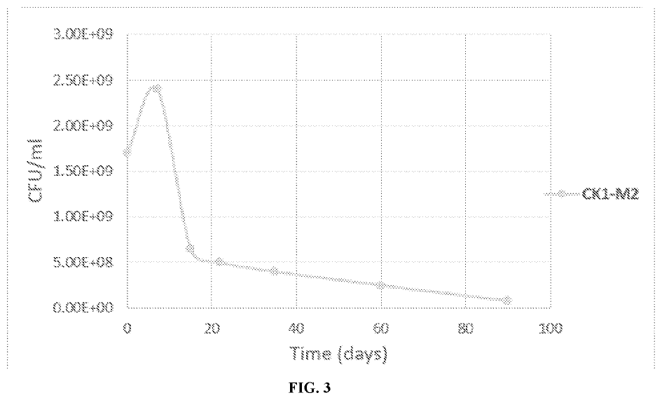

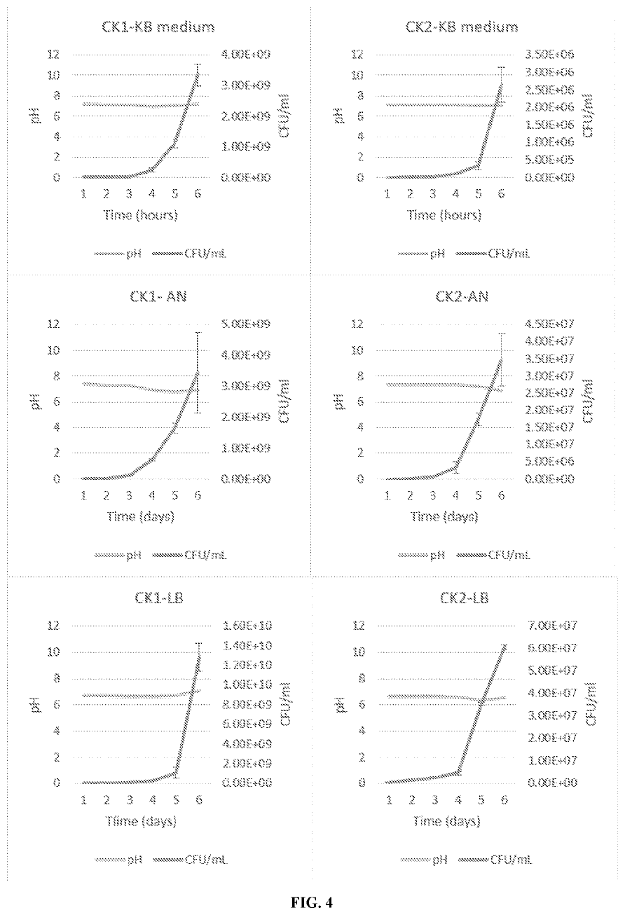

M2 medium leading to CFU/mL values similar to M1 medium for both CK1 and CK2 strains (Table 3). Later, it was also discarded, since acidification was observed, which resulted in a loss of viability in a short time as shown in FIG. 3.

TABLE 3
Parameters evaluated for CK1 and CK2 growth in M2 medium

| | M2 | | |
|---|---|---|---|
| Strain | pH (after culture) | OD600 | CFU/ml |
| Klebsiella aerogenes CK1 | 6.10 | 1.04 | 2.5E+09 |
| Bacillus cereus CK2 | 5.90 | 0.97 | 2.3E+08 |

Due to the results obtained, it was presumed that the sugars (glucose in M1 and sucrose in M2) caused the acidification of the media (see Table 2 and Table 3), which led to the loss of viability, so it was decided to evaluate new culture media without a defined carbon source. Thus, three new culture media (without sugars as C-source) were evaluated for CK1 and CK2 growth.

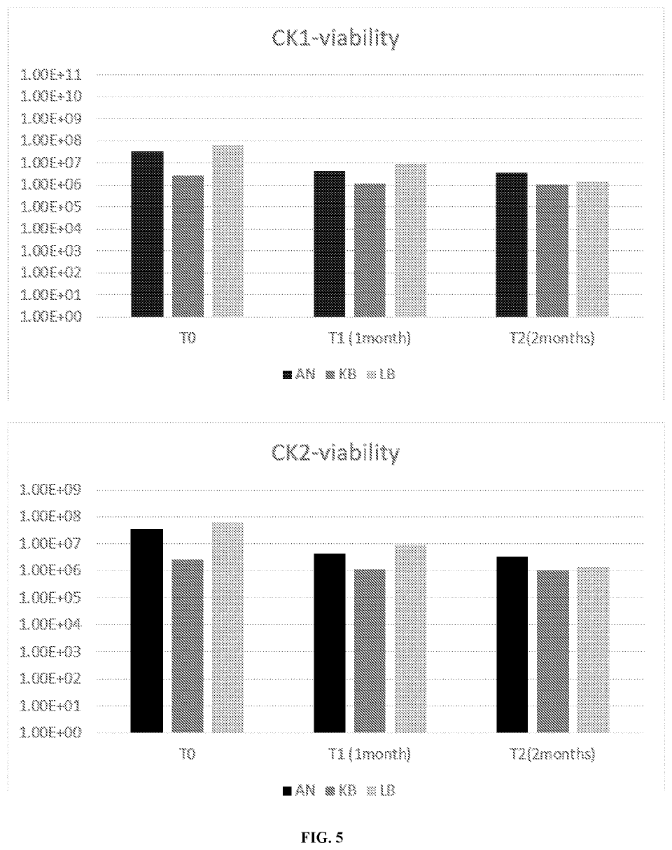

“AN”, “KB” and “LB” medium were the media selected for the assays (the composition was shown in Table 1). The results (FIG. 4) demonstrated that the three culture media evaluated were adequate for the growth of CK1, the bacterial load of 10⁹ CFU/mL was reached in AN and KB while in LB the bacterial count was higher (10¹⁰ CFU/mL). For CK2, LB and AN media were better than KB.

The shelf life for CK1 and CK2 in LB, KB and AN (plus xanthan gum 0.5%) was also evaluated. As shown in FIG. 5, the AN medium was the most suitable and showed better stability in the evaluated time (60 days). In LB medium, even when the higher CK1 CFU/mL value was obtained, the drop in bacterial concentration was large after one month of storage whereas in KB medium the decrease in viability was registered after 30 days.

For CK2, even when CFU/mL drops drastically over time, the AN culture medium seemed to be the most suitable for maintaining higher viable cell numbers at 60 days.

During the 2021 year, the field assays were carried out by both strains growth in AN medium. The product formulated as “CK1+xanthan” or “CK1+CK2+xanthan” was designated as “Extremia A” and “Extremia MIX” respectively.

CK1 and CK2 growth for field testing were carried out in stirred tank reactors (STR), in a volume of 750 L for each strain. A sterile antifoam AF10 silicone was added to the culture media (1/1000) to avoid the generation of high levels of foam, which is a common problem in STR productions due to the high levels of aeration and agitation used.

To obtain the final formulation, the culture broth was mixed with 2% (w/v) xanthan gum (proportion 75% v/v of culture broth+25% v/v of xanthan gum). For Extremia MIX the culture broths for each strain were mixed (50% v/v). Finally, the formulations obtained were packaged in sterile bags of 5 L. The product was stored at room temperature and viability was assessed by measurement of CFU/mL and pH values.

Extremia A reached 1×10⁹ CFU/mL while in extremia MIX the bacteria load was 5×10⁸ CFU/mL for CK1 and 1×10⁸ CFU/mL for CK2.

TABLE 4
Viability of Extremia A and Extremia MIX at monthly interval

| Product | Time | Strain | CFU/ml | pH |
|---|---|---|---|---|
| Extremia | T0 | Klebsiella aerogenes CK1 | 3.6E+08 | 7.45 |
| Mix | | Bacillus cereus CK2 | 1.2E+08 | |
| | T30 days | Klebsiella aerogenes CK1 | 4E+08 | — |
| | | Bacillus cereus CK2 | 1.5E+07 | |
| | T60 days | Klebsiella aerogenes CK1 | 1.84E+08 | 7.75 |
| | | Bacillus cereus CK2 | 1.09E+07 | |
| | T90 days | Klebsiella aerogenes CK1 | 2.6E+07 | 8.36 |
| | | Bacillus cereus CK2 | 6E+06 | |
| Extremia | T0 | Klebsiella aerogenes CK1 | 1.55E+09 | 7.42 |
| A | T30 days | Klebsiella aerogenes CK1 | 2.7E+08 | — |
| | T60 days | Klebsiella aerogenes CK1 | 1.64E+08 | 8.03 |
| | T90 days | Klebsiella aerogenes CK1 | 3.7E+07 | 8.36 |

For field testing the method used for applying the inoculant was the “seed dressing” and the product was mixing with pesticides frequently used. The dose of the Extremia used in the tests was 5 ml per kg of seed.

In a next step, taking into account the drop in CFU/mL after 90 days for both Extremia A and Extremia mix, it was decided to search an alternative culture medium in which a higher CFU/mL count could be achieved, especially for CK2. Thus, new culture media were tested (see Tables 5 and 6). Also, as shown below in Example 2, new formulation alternatives (with adjuvants, cell protector and surfactant) were tested with the aim of getting a better viability in Extremia products.

M3, M4, M5 and M6 culture media (Table 5) were evaluated for CK1 and CK2, nevertheless its use corroborated that the glucose was responsible for dropping the pH value in the medium. As shown in Table 6, in the M4 medium, the pH values remained close to neutrality, and even when CK1 reached a higher CFU/ml, CK2 did not grow enough under those conditions.

To obtain a higher CFU/ml count for CK2, two alternative media, M7 and M8 were tested. In M7 medium the amount of yeast extract was doubled than M4 (1.5 g/L to 3.0 g/L). M8 contained soy peptone instead of yeast extract, which has been described as an alternative and appropriate substrate for Bacillus genus.

TABLE 5
Evaluation of M3, M4, M5, and M6 culture media for
Klebsiella aerogenes CK1 and Bacillus cereus CK2

| Medium | Strain | DO_{600 nm} | pH_{final} | CFU/mL |
|---|---|---|---|---|
| M3 | CK1 | 0.807 | 6.09 | 7.50E+09 |
| M3 | CK2 | 0.787 | 5.01 | 4.50E+08 |
| M4 | CK1 | 0.892 | 7.21 | 1.84E+09 |
| M4 | CK2 | 0.297 | 7.21 | 8.2E+07 |
| M5 | CK1 | 0.96 | 4.78 | 4.90E+09 |
| M5 | CK2 | 0.885 | 4.72 | 9.00E+08 |
| M6 | CK1 | 0.427 | 3.55 | 7.10E+09 |
| M6 | CK2 | 0.857 | 3.89 | 4.30E+07 |

TABLE 6
Evaluation of M6 and M8 for Klebsiella aerogenes
CK1 and Bacillus cereus CK2

| Medium | Strain | DO_{600 nm} | pH | CFU/mL |
|---|---|---|---|---|
| M7 | CK1 | 0.743 | 7.25 | 2.57E+09 |
| M7 | CK2 | 0.598 | 7.07 | 1.06E+08 |
| M8 | CK1 | 0.754 | 7.18 | 2.9E+09 |
| M8 | CK2 | 0.683 | 7.52 | 6.5E+08 |

No significant difference in CK1 growth between M7 and M8 were obtained, however the CFU/ml value for both CK1 and CK2 in M8 medium were slightly higher than M7 medium (Table 4).

M8 culture medium was selected for the growth of both strains (CK1 and CK2) due this allowed the design of a product Extremia mix in which the two bacterial genera are together CK1+CK2.

### Example 2: Cell Protectants

It has been reported that cell protectants are able to improve bacterial homeostasis. Therefore, different cell protectants and additives were evaluated to optimize the shelf life of the products.

The culture media evaluated were AN and M8 described in Table 1. Polymers as polyvinylpyrrolidone (PVP) 2.5% (p/v), Arabic and Xanthan gums were used as cell protectants. Tween 20 2.5% (v/v) was evaluated as surfactant. All additives were added to the culture media during their preparation. The culture media were sterilized by autoclave (15 min).

Xanthan gum and Arabic gum were dissolved in physiological solution (NaCl 8 g/l).

Inoculation: Before inoculating, sterile AF10 silicone antifoam was added to the culture media (1/1000).

Incubation: Culture media were incubated at 30° C., 200 rpm. 8 hours and after growth CFU/ml of the culture broths of each strain (CK1, CK2) are made.

The different formulations were prepared as described below:
A) Xanthan gum (2% w/v). Proportion: Xanthan 25%-75% culture broth. Xanthan concentration in the formulation: 0.5%.
B) Carboxymethylcellulose (CMC, 1% w/v). Proportion: CMC 25%-75% culture broth. CMC concentration in the formulation: 0.25%.
C) Nothing was added.

The product was packaged in plastic bottles and stored at room temperature protected from light.

TABLE 7
Carrier formulation materials for microbial inoculant

| Culture media | Strain | Additive |
|---|---|---|
| M8 + G. Arabic 0.6% (p/v) + Tween 0.025% (v/v) | Klebsiella | none |
| | aerogenes CK1 | |
| M8 + G. Arabic 0.6% (p/v) + Tween 0.025% (v/v) | Klebsiella | Xanthan 2% |
| | aerogenes CK1 | (p/v) en FS |
| M8 + G. Arabic 0.6% (p/v) + Tween 0.025% (v/v) | Klebsiella | CMC 1% (p/v) |
| | aerogenes CK1 | en FS |
| M8 + G. Arabic 0.6% (p/v) + Tween 0.025% (v/v) | MIX (Klebsiella | none |
| | aerogenes CK1. | |
| | Bacillus cereus | |
| | CK2) | |
| M8 + G. Arabic 0.6% (p/v) + Tween 0.025% (v/v) | MIX (Klebsiella | Xanthan 2% |
| | aerogenes CK1. | (p/v) en FS |
| | Bacillus cereus | |
| | CK2) | |
| M8 + G. Arabic 0.6% (p/v) + Tween 0.025% (v/v) | MIX (Klebsiella | CMC 1% (p/v) |
| | aerogenes CK1. | en FS |
| | Bacillus cereus | |
| | CK2) | |
| M8 + PVP 2.5% (p/v) + Tween 0.025% (v/v) | Klebsiella | none |
| | aerogenes CK1 | |
| M8 + PVP 2.5% (p/v) + Tween 0.025% (v/v) | Klebsiella | Xanthan 2% |
| | aerogenes CK1 | (p/v) en FS |
| M8 + PVP 2.5% (p/v) + Tween 0.025% (v/v) | Klebsiella | CMC 1% (p/v) |
| | aerogenes CK1 | en FS |
| M8 + PVP 2.5% (p/v) + Tween 0.025% (v/v) | MIX (Klebsiella | none |
| | aerogenes CK1. | |
| | Bacillus cereus | |
| | CK2) | |
| M8 + PVP 2.5% (p/v) + Tween 0.025% (v/v) | MIX (Klebsiella | Xanthan 2% |
| | aerogenes CK1. | (p/v) en FS |
| | Bacillus cereus | |
| | CK2) | |
| M8 + PVP 2.5% (p/v) + Tween 0.025% (v/v) | MIX (Klebsiella | CMC 1% (p/v) |
| | aerogenes CK1. | en FS |
| | Bacillus cereus | |
| | CK2) | |
| AN + NaCl 0.8% + G. Arabic 0.6% (p/v) + Tween | Klebsiella | none |
| 0.025% (v/v) | aerogenes CK1 | |
| AN + NaCl 0.8% + G. Arabic 0.6% (p/v) + Tween | Klebsiella | Xanthan 2% |
| 0.025% (v/v) | aerogenes CK1 | (p/v) en FS |
| AN + NaCl 0.8% + G. Arabic 0.6% (p/v) + Tween | Klebsiella | CMC 1% (p/v) |
| 0.025% (v/v) | aerogenes CK1 | en FS |
| AN + NaCl 0.8% + G. Arabic 0.6% (p/v) + Tween | MIX (Klebsiella | none |
| 0.025% (v/v) | aerogenes CK1. | |
| | Bacillus cereus | |
| | CK2) | |
| AN + NaCl 0.8% + G. Arabic 0.6% (p/v) + Tween | MIX (Klebsiella | Xanthan 2% |
| 0.025% (v/v) | aerogenes CK1. | (p/v) en FS |
| | Bacillus cereus | |
| | CK2) | |
| AN + NaCl 0.8% + G. Arabic 0.6% (p/v) + Tween | MIX (Klebsiella | CMC 1% (p/v) |
| 0.025% (v/v) | aerogenes CK1. | en FS |
| | Bacillus cereus | |
| | CK2) | |
| AN + NaCl 0.8% + PVP 2.5% (p/v) + Tween 0.025% | Klebsiella | none |
| (v/v) | aerogenes CK1 | |
| AN + NaCl 0.8% + PVP 2.5% (p/v) + Tween 0.025% | Klebsiella | Xanthan 2% |
| (v/v) | aerogenes CK1 | (p/v) en FS |
| AN + NaCl 0.8% + PVP 2.5% (p/v) + Tween 0.025% | Klebsiella | CMC 1% (p/v) |
| (v/v) | aerogenes CK1 | en FS |
| AN + NaCl 0.8% + PVP 2.5% (p/v) + Tween 0.025% | MIX (Klebsiella | none |
| (v/v) | aerogenes CK1. | |
| | Bacillus cereus | |
| | CK2) | |
| AN + NaCl 0.8% + PVP 2.5% (p/v) + Tween 0.025% | MIX (Klebsiella | Xanthan 2% |
| (v/v) | aerogenes CK1. | (p/v) en FS |
| | Bacillus cereus | |
| | CK2) | |
| AN + NaCl 0.8% + PVP 2.5% (p/v) + Tween 0.025% | MIX (Klebsiella | CMC 1% (p/v) |
| (v/v) | aerogenes CK1. | en FS |
| | Bacillus cereus | |
| | CK2) | |

The additives were evaluated in different proportions according to the bibliography, as shown in Table 8. The selection of the best concentration was made based on the CFU/mL count obtained compared to the control (medium without additives).

In all cases the lowest concentration in which growth was greater or the same than the control was selected.

TABLE 8
Additives and concentration used

| Properties | Additives |
|---|---|
| Cell protectants | Glycerol 5 mM |
| Cell protectants | Glycerol 10 mM |
| Cell protectants | Glycerol 15 mM |
| Cell protectants | Glycerol 20 mM |
| Surfactant | Tween 20 0.025% |
| Surfactant | Tween 20 0.5% |
| Protectant | Arabic gum 0.6% |
| Protectant | Arabic gum 0.8% |
| Preservative | Potassium sorbate 0.2% |
| Protectant | PVP 2.5% |
| Protectant | PVP 2% |
| Protectant | Sodium Alginate 0.1% |
| Adjuvant | CMC 0.1% |
| Adjuvant | Xanthan 2% |

The best counts (CFU/mL) were obtained with the combination of tween, Arabic gum and PVP. It is important to note that the pH values remained close to neutrality. These results were not obtained with glycerol, which produced acidification in the culture medium. Potassium sorbate affected the growth of CK2, this strain grew less in its presence, so it was discarded and, on the other hand this protectant combined with PVP alkalinized the medium with CK1.

TABLE 9
Effect of different additives on the population of
Klebsiella aerogenes CK1 and Bacillus cereus CK2

| | Product | Protectant | Surfactant | CFU/ml | pH |
|---|---|---|---|---|---|
| 1 | Klebsiella aerogenes | PVP 2.5% | Sorbitol K 0.2% | 1.8 × 10⁹ | 7.73 |
| | CK1 | | | | |
| 1 | Bacillus cereus CK2 | | | 1.9 × 10⁷ | 6.61 |
| 3 | Klebsiella aerogenes | Alginate 0.1% | Sorbitol K 0.2% | 1 × 2 × 10⁹ | 6.97 |
| | CK1 | | | | |
| 3 | Bacillus cereus CK2 | | | 2 × 10⁷ | 6.55 |
| 5 | Klebsiella aerogenes | Glycerol 5% | Sorbitol K 0.2% | 4.15 × 10⁸ | 5.60 |
| | CK1 | | | | |
| 5 | Bacillus cereus CK2 | | | 6.7 × 10⁷ | 6.05 |
| 7 | Klebsiella aerogenes | PVP 2.5% | Tween20 0.025% | 1.1 × 10⁹ | 6.82 |
| | CK1 | | | | |
| 7 | Bacillus cereus CK2 | | | 1.54 × 10⁸ | 6.56 |
| 8 | Klebsiella aerogenes | Arabic gum 0.6% | Tween20 0.025% | 1.9 × 10⁹ | 6.67 |
| | CK1 | | | | |
| 8 | Bacillus cereus CK2 | | | 1.4 × 10⁸ | 6.49 |
| CTRL | Klebsiella aerogenes | — | — | 1.6 × 10⁹ | 7.06 |
| | CK1 | | | | |
| CTRL | Bacillus cereus CK2 | — | — | 1.6 × 10⁸ | 6.52 |

The process was simplified by adding all the components to the culture medium before inoculating. At the end of the process the xanthan gum or CMC were added.

To select the best formulation, CFU/mL were evaluated every month, as shown in Table 10. The addition of Arabic gum/PVP, tween 20 and xanthan/CMC improved the stability of the product, especially for CK1 in the Mix product. Spores' formation was identified for CK2 during time.

TABLE 10
Effect of additives on the pH and CFU/mL of Klebsiella aerogenes
CK1 and Bacillus cereus CK2 during time.

| | | | | | | | |
|---|---|---|---|---|---|---|---|
| | | M8 + GA + | M8 + GA + T + | M8 + GA + | M8 + PVP + | M8 + PVP + | M8 + PVP + |
| Days | M8 + XANT | T | XANT | T + CMC | T | T + XANT | T + CMC |

CK1 A/M8

| | | | | | | | |
|---|---|---|---|---|---|---|---|
| 0 | 1.22E+09 | 1.73E+09 | 1.10E+09 | 1.47E+09 | 1.78E+09 | 1.65E+09 | 1.80E+09 |
| 30 | 3.00E+07 | 3.00E+07 | 1.10E+08 | 1.85E+08 | 1.85E+07 | 1.20E+08 | 1.21E+08 |
| 60 | 4.75E+07 | 1.60E+08 | 2.20E+08 | 1.75E+08 | 3.80E+07 | 1.08E+08 | 3.70E+07 |

pH

| | | | | | | | |
|---|---|---|---|---|---|---|---|
| 0 | | 6.99 | 7.05 | 6.9 | | 6.95 | 7.25 |
| 30 | 7.27 | 7.47 | 7.22 | 7.4 | 7.76 | 8.23 | 8.45 |
| 60 | 8.35 | 7.69 | 7.45 | 7.48 | 7.98 | 8.17 | 8.12 |

CK1 + CK2/M8

| | | | | | | | |
|---|---|---|---|---|---|---|---|
| 0 | 1.73E9 | 9.40E+08 | 4.97E+08 | 4.10E+08 | 2.13E+09 | 1.33E+09 | 1.27E+09 |
| 30 | 3.00E+07 | 4.30E+07 | 5.10E+08 | 2.90E+08 | 4.50E+08 | 7.50E+08 | 2.90E+08 |
| 60 | 1.85E+07 | 3.00E+07 | 1.2E8 | 2.60E+08 | 3.40E+07 | 1.90E+08 | 1.60E+08 |

Days/CK2

| 0 | 6.50E+07 | 7.70E+07 | 4.00E+07 | 3.20E+07 | 2.35E+07 | 4.35E+07 | 2.50E+07 |
|---|---|---|---|---|---|---|---|
| 30 | — | 1.80E+06 | 4.25E+06 | 1.80E+06 | — | 2.50E+06 | 2.00E+07 |
| 60 | — | 2.00E+06 | — | 1.00E+06 | 1.10E+06 | 1.00E+05 | 3.10E+06 |
| | | AN + GA + | AN + GA + T + | AN + GA + | AN + PVP + | AN + PVP + | AN + PVP + |
| Days | AN + XANT | T | XANT | T + CMC | T | T + XANT | T + CMC |

CK1/AN

| | | | | | | | |
|---|---|---|---|---|---|---|---|
| 0 | 1.40E+09 | 2.43E+09 | 2.30E+09 | 2.00E+09 | 2.50E+09 | 2.50E+09 | 2.00E+09 |
| 30 | 1.44E+08 | 3.30E+08 | 4.00E+07 | 2.3E+08 | 2.20E+08 | 4.00E+07 | 3.00E+08 |

pH

| | | | | | | |
|---|---|---|---|---|---|---|
| 0 | 6.72 | 6.94 | 6.88 | 6.75 | 6.68 | 6.85 |
| 30 | 6.87 | 6.93 | 7.01 | 7.07 | 6.88 | 6.99 |

CK1 + CK2/AN

| | | | | | | | |
|---|---|---|---|---|---|---|---|
| 0 | 1.38E+08 | 7.50E+08 | 1.16E+09 | 1.00E+09 | 1.14E+09 | 1.46E+09 | 1.06E+09 |
| 30 | | 2.00E+08 | 1.5E8 | 4.00E+08 | 4.44E+07 | 4.40E+07 | 7.00E+07 |

Days/CK2

| | | | | | | | |
|---|---|---|---|---|---|---|---|
| 0 | 2.57E+07 | 4.60E+06 | 3.00E+07 | 1.00E+07 | 2.00E+06 | 7.50E+07 | 3.00E+06 |
| 30 | | 3.1E7 | >1E4 | 1.00E+07 | <1E4 | 2.00E+04 | <1E4 |

The results showed that in M8 medium “M8+GA+T+XANT” was the best combination evaluated. Although in the first month it experienced a drop in the CFU/mL, then the pH values were maintained in this formulation. This was not observed with the use of “M8+PVP+T+XANT” and “M8+PVP+T+XANT” in which the medium was alkalinized, values greater than 8 were reported.

Regarding CK1 and CK1+CK2 in AN medium, the best results were reported with CM.

The combinations “AN+GA+T+XANT” and “AN+PVP+T+XANT” were not effective. Furthermore, the results reported shown that the use of PVP in CK1+CK2 was not useful. At 30 days the CFU/mL counts were less than 1E4.

### Example 3: CK2 Sporulation

Bacterial spores have been defined as small oval structures exhibiting a strong resistance to high temperatures, desiccation, radiation, and chemical agents. Products based on spores could improve the survival of the bacteria over time, thus enhancing products shelf life, and the resistance of the product to stress conditions. Therefore, CK2 sporulation capacity was evaluated.

CK2 was grown in different media and then evaluated for the induction of sporulation (see protocol above). CK2 cultures were grown, and CFU/mL count and pH were measured as described in Example 1. The CK2 morphology was controlled using an optical microscope Samples were measured at 24 hrs, 48 hrs, and 72 hrs.

The culture media tested, and results are shown in Table 11 and 12 respectively.

### Sporulation Protocol: TOTAL CELLS AND SPORES COUNT

The number of total cells (vegetative cells+germinative cells or spores) and the number of spores produced in each tested condition were evaluated at different times. CFU/mL count was performed before and after heating the sample following the next protocol:

1 mL of the growth medium containing CK2 was sampled.

100 μL were used to perform CFU/mL.

The remaining sample was heated at 70° C., or 80° C. for 20 min.

The spore count (CFU/mL) of the heated sample was measured. Overall, one less dilution should be plated than in the count without heating, unless the entire sporulated culture is observed under a microscope, in which case the same dilutions should be plated.

TABLE 11
Culture Media for Spore Production in Bacillus cereus CK2

| (g/L) | Glucose | Soy flour | Cornstarch | MgSO₄•7H₂O | MnSO₄ | CaCl |
|---|---|---|---|---|---|---|
| S1 | 1.2 | — | — | 0.5 | — | — |
| S2 | 1.2 | — | — | 0.5 | — | — |
| S3 | 1.2 | 35 | 30 | 0.3 | 0.3 | 3 |
| S4 | 1.2 | — | — | 0.5 | 0.3 | — |
| S5 | 2.41 | 10.13 | 16.89 | 0.45 | — | — |
| S6 | 2 | 9.5 | 9 | — | 0.3 | 1.55 |
| S7 | — | — | — | — | — | 5 |
| S8 | — | — | — | — | — | — |
| S9 | — | — | 11.1 | — | — | 5 |
| S10 | — | — | 12.5 | — | — | — |
| S11 | — | — | — | 0.15 | 0.04 | 0.04 |
| S12 | — | — | — | — | — | — |
| S13 | — | | | | | |
| S15 | 1.2 | — | — | 0.5 | 0.04 | — |
| S16 | — | — | — | 0.2 | — | — |
| S17 | — | — | — | 0.5 | 0.04 | — |
| S18 | 1.2 | — | — | — | 0.04 | — |
| S19 | 1.2 | — | — | 0.5 | 0.04 | — |
| S20 | 1.2 | — | — | 0.5 | — | — |
| S21 | 1.2 | — | — | 0.5 | 0.04 | — |
| S22 | 1.2 | — | — | 0.5 | 0.04 | — |
| S23 | — | — | — | 0.5 | — | — |
| AN | — | — | — | — | — | — |

| | | | | | | Soluble |
|---|---|---|---|---|---|---|
| (g/L) | Meat ext. | KH₂PO₄ | Triptein | (NH₄)₂HSO₄ | Na Cl | potato starch |
| S1 | 5 | — | 3 | — | — | — |
| S2 | 3 | — | — | — | — | — |
| S3 | — | — | — | — | — | — |
| S4 | 5 | 6 | 3 | — | — | — |
| S5 | — | — | 1.13 | 7.96 | 4.5 | — |
| S6 | 7.2 | — | — | — | — | — |
| S7 | — | — | — | — | — | 11.1 |
| S8 | — | — | — | 1 | — | 12.5 |
| S9 | — | — | — | — | — | — |
| S10 | — | — | — | 1 | — | — |
| S11 | — | 1 | 5 | — | — | — |
| S12 | 2.5 | — | — | — | — | — |
| S13 | 3.75 | | | | | |
| S15 | — | — | — | — | — | — |
| S16 | — | — | — | 2 | — | — |
| S17 | 1.5 | — | — | — | — | — |
| S18 | 1.5 | — | — | — | — | — |
| S19 | 1.5 | — | — | — | — | — |
| S20 | 1.5 | — | — | — | — | — |
| S21 | 1.5 | — | — | — | — | — |
| S22 | — | — | — | — | — | — |
| S23 | 1.5 | — | — | — | — | — |
| AN | 1.5 | — | — | — | — | — |

| | | | | | Sodium |
|---|---|---|---|---|---|
| (g/L) | FeCl₃•6H₂O | Yeast Ext. | K₂HPO₄ | Pluripeptone | citrate |
| S1 | — | — | 6 | — | — |
| S2 | — | — | 6 | 5 | — |
| S3 | — | — | — | — | — |
| S4 | — | — | — | — | — |
| S5 | — | — | — | — | — |
| S6 | — | — | — | — | — |
| S7 | — | 3 | — | — | — |
| S8 | — | 10 | — | — | — |
| S9 | — | 3 | — | — | — |
| S10 | — | 10 | — | — | — |
| S11 | 0.3 | 3 | — | — | — |
| S12 | — | — | — | 1.5 | — |
| S13 | | | — | 2.25 | — |
| S15 | 0.3 | 1.5 | 1 | 2.5 | — |
| S16 | — | — | 0.125 | — | 7.5 |
| S17 | 0.3 | — | 1 | 2.5 | — |
| S18 | 0.3 | — | 1 | 2.5 | — |
| S19 | 0.3 | — | — | 2.5 | — |
| S20 | 0.3 | — | 1 | 2.5 | — |
| S21 | — | — | 1 | 2.5 | — |
| S22 | 0.3 | — | 1 | — | — |
| S23 | 0.3 | — | 1 | 2.5 | — |
| AN | — | — | — | 2.5 | — |

TABLE 12
Evaluation of Spore Production in
Bacillus cereus CK2 (First Stage)

| Culture | | Incubation | | | |
|---|---|---|---|---|---|
| media | Strain | time | pH | CFU/mL | Spores/mL |
| S1 | CK2 | 24 h | 7.15 | 8.00E+08 | 2.7E+05 |
| | | 48 h | — | 1.5E+08 | 2.00E+04 |
| S2 | CK2 | 24 h | 7.08 | 1.36E+09 | — |
| | | 48 h | — | 2.8E+08 | — |
| S3 | CK2 | 24 h | 4.83 | 1.3E+08 | — |
| | | 48 h | 6.06 | 9.00E+08 | — |
| | | 72 h | — | — | — |
| S4 | CK2 | 24 h | 6.43 | 1.25E+08 | — |
| | | 48 h | 6.72 | 3.96E+08 | 4.00E+05 |
| | | 72 h | 7.30 | 6.6E+07 | 2.00E+07 |
| S5 | CK2 | 24 h | 4.84 | 1.00E+08 | — |
| | | 48 h | 4.83 | 8.00E+06 | — |
| | | 72 h | — | — | — |
| S6 | CK2 | 24 h | 4.84 | 3.58E+08 | 2.00E+06 |
| | | 48 h | 4.79 | 6.00E+06 | — |
| | | 72 h | — | — | — |
| S7 | CK2 | 24 h | 4.57 | 1.1E+07 | — |
| | | 48 h | 4.51 | — | 5.00E+06 |
| | | 72 h | 4.69 | — | 1.00E+06 |
| S8 | CK2 | 24 h | 5.92 | 4.35E+08 | — |
| | | 48 h | 5.92 | 3.00E+07 | 2.00E+07 |
| | | 72 h | — | — | — |
| S9 | CK2 | 24 h | 5.81 | 1.00E+08 | — |
| | | 48 h | 5.83 | 1.00E+08 | — |
| | | 72 h | — | — | — |
| S10 | CK2 | 24 h | 5.28 | 2.18E+08 | — |
| | | 48 h | 5.18 | 6.00E+06 | 3.00E+07 |
| | | 72 h | 5.06 | 3.2E+07 | 6.5E+06 |
| S11 | CK2 | 24 h | — | 2.25E+08 | 1.7E+06 |
| | | 48 h | 6.91 | 2.00E+08 | 6.00E+06 |
| | | 72 h | 7.63 | 1.00E+08 | 3.00E+06 |
| S12 | CK2 | 24 h | 6.67 | 2.18E+08 | 3.00E+06 |
| | | 48 h | — | 1.00E+08 | 1.00E+05 |
| | | 72 h | 7.01 | 1.49E+08 | 1.4E+05 |
| S13 | CK2 | 24 h | 6.46 | 7.00E+07 | 3.00E+06 |
| | | 48 h | — | 1.26E+08 | — |
| | | 72 h | 7.07 | 1.1E+08 | 6.2E+05 |

The first culture media evaluated for CK2 sporulation were S1 and S2. The results obtained in S1 and S2, indicated an optimal CFU/mL count and the presence of some refringent spores inside the CK2 vegetative cells under a microscope. However, CK2 was not able to sporulate.

Since nutrient deprivation (C, N and/or P) has been described a key factor to trigger bacterial sporulation, these results could be linked to an excess of nutrients in the culture media, which prevented them from becoming limiting in the times evaluated and therefore the sporulation could not take place.

Regarding the pH value, it remained neutral. It has been shown that in some Bacillus species, the sporulation process depends on the pH value. Nevertheless, in other cases, it was possible to work at a free pH without affecting the sporulation capacity of the bacteria. Despite these differences, overall, it has been reported that the sporulation process increases the pH of the medium above 7.5.

Further studies are needed to elucidate the influence of the pH on the sporulation process of the CK2 strain.

Regarding the culture media S3, S4, S5, S6, S7, S8, S9, S10, S11, S12 and S13, an optimal CFU/mL count was reached. However, in most of them, the spores number obtained was low.

Particularly, S3, S5, S6 and S9, exhibited no spores after 48 hours of incubation, thus they were discarded.

Despite S8 medium, showed 2.00E+07 spores/mL, it was discarded since it was composed of soluble potato peptone as a sole carbon source. Potato peptone is manufactured by a controlled enzymatic hydrolysis of potato proteins, and its composition usually changes between production batches, changing the spore's concentration in the culture medium. In addition, its commercial availability was low. Therefore, corn starch was added (S9 culture medium) as a replacement of potato peptone. However, no spore formation was observed in S9, under the studied conditions.

After 48 hours, S4, S6, S10 and S11 reached 4.00E+06, 5.00E+06, 3.00E+07 and 6.00E+07 spores/mL, respectively. At 72 hours, S4 medium exhibited an increment of the spore's quantity. No changes were obtained for S7 and S11 culture media. In contrast, spores/mL decreased in the S10 medium.

Even though spore's production in S12 and S13 was evaluated up to 72 hours, the highest number of spores/mL was registered at 24 hours.

Regarding the pH, it has been observed that the higher the spores/mL, the higher the pH value, when CK2 was grown in S4 medium. Conversely, no pH changes were registered in S10 medium. Furthermore, the pH values were lower (around 5) than the ones obtained in S4.

Finally, S11, S12 and S13 showed an increase in pH over time. As it was mentioned before, it has been reported that the bacterial sporulation process usually provokes a culture medium alkalinization raising the pH value.

In spite of having found some culture media capable of exhibiting spores' formation, the concentration of them was still low. Therefore, new culture media was evaluated.

CK2 was grown in S15, S16 and AN media. The protocol followed to grow CK2 was slightly different from the one previously described. In all cases, it was decided to use CaCl2 1.55 g/L (2 Mm) as inducer of the sporulation process. Thus, CK2 was incubated at 30° C., and 230 rpm, to obtain a CK2 culture in stationary phase. At 20 hours, the inducer (CaCl2 1.55 g/L) was added, and sporulation was evaluated at different times: 0, 2, 4, 6, 24 hours and 4 days after the addition of the inducer.

TABLE 13
Evaluation of spore production in
Bacillus cereus CK2 (second stage)

| Time (h) | spores/mL | spores/mL | spores/mL |
|---|---|---|---|
| Inducer aggregate | S15 | S16 | AN |
| 0 | 1.1E+05 | 1.83E+07 | 1.5E+05 |
| 2 | 4.5E+05 | 1.1E+07 | 2.5E+06 |
| 4 | 1.6E+08 | 1.1E+07 | 3.6E+07 |
| 6 | 2.2E+08 | 1.2E+07 | 1.9E+07 |
| 24 | 2.4E+08 | 8.0E+06 | 3.31E+08 |
| 96 | 4.0E+08 | 1.6E+07 | 1.71E+08 |

According to the results (Table 13), although CK2 sporulation was quickly reached in S16 culture medium, the growth of the strain was low (1.0E+7). The pH value registered was 7.4 at 24 hours after adding the inducer and 8.5 after 4 days. Free spores were found in the culture medium at 4 days of growth. In addition, no induction of the sporulation process was observed after adding CaCl2. Since CK2 growth was inferior than expected, and the spores concentration was lower than the values reached in S15 and AN, the use of S16 was discarded.

Regarding S15 and AN culture media, the results showed that the sporulation process was triggered a few hours earlier in S15 compared to AN medium. In both culture media, the addition of CaCl2 was able to induce the sporulation in CK2.

According to the results, AN optimization was abandoned. In contrast, it was decided to continue working on the optimization of the medium S15.

In a third stage, the effect of adding a higher volume of the initial inoculum in the process was tested. Two different conditions were compared following the next protocol:

### Condition 1

Approximately 100 mL of S15 medium were placed in a 250 mL Erlenmeyer flask. Erlenmeyer flask was inoculated with 1 mL (0.1%) of CK2, previously grown in S15 culture grown (approx. 20 hours). Erlenmeyer flask was then incubated during 18h, at 30° C., and 230 rpm, to obtain a CK2 culture in stationary phase. After 18 hours of culture, the inducer was added (CaCl2 1.55 g/L, approx. 2 mM). The following incubation was performed at 30° C., 230 rpm and the sporulation of the culture was evaluated at different times: 0, 2, 4, 6, 8, 24 and 48 hours after the addition of the inducer.

### Condition 2:

The steps were the same described for condition 1, but the Erlenmeyer flask was inoculated with 10 mL (10%) of CK2 instead of 1 ml.

The results are shown in Table 14. Under the tested conditions, the volume of the initial CK2 inoculum affected the sporulation process. The increase of the volume of the initial CK2 inoculum (from 0.1% to 10%) accelerated the sporulation process. Total sporulation was achieved two hours after the addition of CaCl2. In contrast, when 0.1% of the inoculum was used, complete sporulation was reached after 24 hours of adding CaCl2.

Therefore, hereinafter the initial CK2 inoculum was used at 10%, regardless the final culture volume.

TABLE 14
Evaluation of spore production in Bacillus cereus CK2 (third stage)

| | S15 | | S15 | |
|---|---|---|---|---|
| Time (h) | 10% initial inoculum | | 0.1% initial inoculum | |
| Inducer | | Sporulation | | Sporulation |
| aggregate | Spores/mL | percentage (%)* | Spores/mL | percentage (%) |
| 0 | 1.3E+07 | 7.3% | 2.8E+05 | ≤1% |
| 2 | 5.9E+08 | 100% | 1.0E+05 | ≤1% |
| 4 | 4.93E+08 | 100% | 3.0E+06 | 1.3% |
| 6 | 5.72E+08 | 100% | 2.0E+07 | 10% |
| 8 | 6.6E+08 | 100% | 7.5E+07 | 34% |
| 24 | 3.5E+08 | 70% | 3.7E+08 | 100% |
| 48 | 3.4E+08 | 85% | 2.0E+08 | 100% |

*Sporulation percentage (%): Sporulation efficiency (%) = (Final number of spores/Final number of total cells)*100

Finally, in a fourth stage, the optimization of the S15 culture medium was performed. The influence of the different components was evaluated. In addition, the inducer (CaCl2) was added to the culture medium at the beginning of the growth. The incubation was stablished at 30° C., 230 rpm during 18 h. The parameters evaluated are described in Table 15.

TABLE 15
Evaluation of spore production in
Bacillus cereus CK2 (fourth stage)

| Culture | Initial | Final | | |
|---|---|---|---|---|
| media | pH | pH | CFU/mL | Spores/mL |
| S15 | 7.5 | 7.4 | 1.81E+08 | 2.8E+8 |
| S17 | 7.5 | 7.5 | 1.6E+08 | 3.6E+08 |
| S18 | 7.5 | 7.25 | 1.81E+08 | 1E+06 |
| S19 | 7.5 | 7.60 | 1.93E+08 | 2.5E+08 |
| S20 | 7.5 | 7.36 | 2E+08 | 6.4E+08 |
| S21 | 7.5 | 7.24 | 2E+08 | 4E+06 |
| S22 | 7.5 | 5.98 | 2E+07 | 2.3E+07 |
| S23 | 7.5 | 7.69 | 2.53+08 | 7E+08 |

According to the results, the cations Mg2+ and Fe3+, showed to be important in the composition of the S15 medium since removing any of the cations the sporulation process was delayed. The spore count in the S17, S19 and S20 media (without glucose, dibasic phosphate, and manganese, respectively) was comparable to the S15 medium. In contrast, S18 and S21 showed a lower number of spores compared to S15. On the other hand, S22 (without pluripeptone and meat extract), exhibited a spore count one order lower than the control (S15). However, CK2 growth was also lower in this condition.

Regarding the pH value, in all culture media (including the control) it remained between 7.24 and 7.60, except for S22, in which a decrease in pH (5.98) was observed. It could be explained by the presence of glucose in the culture medium since its metabolism induced the acidification of the culture medium.

Overall, the presence of Fe3+ and Mg2+ ions was important to reach the sporulation of CK2 within 18 hours of growth. The same was found for pluripeptone and meat extract. In contrast, glucose, Mn2+ and/or dipotassium phosphate could be removed from the S15 medium, without negatively affecting the growth and sporulation process. Therefore, S17 medium was selected to perform the last optimization removing the Mn2+ ions.

As it shown in Table 15, S23 culture medium exhibited the highest number of spores as well as the greater growth. Thus, S23 was chosen as the sporulation culture medium for CK2 and to evaluate the viability over time of the strain.

### Example 4: Field Testing—Extremia A and Extremia MIX Treated Seeds Improve Crop Yields

Field trials were performed with either the Extremia A or Extremia MIX.

Products were manufactured by growing the bacteria in AN culture medium, containing 10 g×L⁻¹ peptone, 10 g×L⁻¹ meat extract and 5 g×L⁻¹ sodium chloride. Growth conditions were settled as follows:
Temperature: 30° C.
Growth time: between 6 and 8 hours.
Bacterial final concentration: Extremia A, containing CK1 strain, reached 1×10⁹ CFU/mL. Extremia MIX presented 3×10⁸ CFU/mL of CK1 and 1×10⁸ CFU/mL of CK2. For Extremia MIX the culture broths for each strain were mixed (50% v/v).

The cultures were then mixed with the stabilizer in a ratio of 7.5:2.5 (vol./vol.) respectively.

The stabilizer selected for the mixture was xanthan gum FF fine food (Phernhofen). At the beginning the xanthan gum was prepared at 2% (weight/vol.) using distilled water to dissolve it. Later, the dissolvent was replaced by physiological solution (NaCl 8 g×L⁻¹) since it helps to stabilize the osmotic pressure in the medium.

The initial preparation of the xanthan gum included a magnetic stirring and dissolution of remaining lumps with a spoon. Currently, a propeller stirrer (Dlab brand, model OS40-S) is used at 1000-1400 rpm (approx.) until complete dissolution.

An antifoam (AF10 silicone from Permaquim) is mixed with the xanthan gum before sterilizing. The antifoam is diluted 1/1000.

The sterilization of the xanthan gum with the silicone is performed in an autoclave. The sterilization time depends on the volume of xanthan gum placed in the container. Generally, 500 mL is placed and sterilized for at least 20 minutes to ensure sterility.

Finally, the mixture was packed in either sterile 100 mL plastic bottles or 2 L/5 L plastic bladders and preserved at room temperature (˜25° C.).

Soybean seeds were inoculated with either the Extremia A Product or Extremia MIX, a commercial Bradyrhizobium inoculant (CKC liquid soybean, 0.96 mL per kg of seed) or a combination of Extremia A and Bradyrhizobium (2.5 mL and 0.48 mL respectively).

Control treatment seeds were not inoculated. Seeds were inoculated with either Extremia A Product or Extremia MIX at 5 mL of inoculant per kg of seed or 6 mL of inoculant per kg of seed.

Seed dressing was the method used by seed treatment. The liquid formulation was mixed with pesticides frequently used. The seeds were treated with the liquid inoculant according to the producer: it can be mechanical or manual (using different containers to make the mixture).

The treated seeds were grown under different locations and conditions, and the subsequent development of the crops was evaluated.

Seeds were planted, grown, and harvested under various environmental and soil conditions representative of potential agricultural conditions. The locations and conditions are listed in Table 16.

Seed planted was carried out with an experimental direct seeders machine at 52 cm between rows.

Four central rows were harvested, and Yield (kg/ha) was determined in each Experimental unit (Number of pods per plant, N° grains/pod and grain weight). A sample (1 kg) of the harvested seeds from each experimental unit was subjected to quality analysis.

TABLE 16
Seed growth conditions

| | Previous | | | rainfall | | Phosphorus | |
|---|---|---|---|---|---|---|---|
| Location | crop | Soil types | Fertilization | cycle | M.O % | ppm | pH |
| Tucuman | Corn | Typical | yes | Normal | 3.36 | 17.3 | 7.66 |
| | | hapludoll | | | | | |
| San Jeronimo | Barley | | Yes | Normal | 2.3 | 19 | 5.83 |
| Norte | | | | | | | |
| F. Ameghino | Corn | Hapludoll | Yes | Stress | | 9.5 | |
| Bs As | | | | | | | |
| Pergamino | Corn | Typic | Yes | Low | 3 | 14.6 | 5.6 |
| Bs As | | argiudolls | | | | | |
| Tres Arroyos | Barley | Petrocalcic | yes | Stress | 3.4 | 9 | 6.1 |
| Bs As | | Argiudolls | | | | | |
| Colonia | Corn | | No | Stress | N/A | N/A | N/A |
| Caroya CBA | | | | | | | |
| Diamante ER | Wheat | Aquic | No | Low | 2.6 | 58.1 | 6.2 |
| | | argiudolls | | | | | |

Results are shown in Table 17. The data showed that all treatments with Extremia A and Extremia MIX Products had average increased yield between 9.8% and 16.7% over untreated seed, compared to 3.9% increased yield of the commercial Bradyrhizobium product evaluated.

To calculate yield, the four central rows (8 meters long) of each experimental unit were harvested. Measurements of yield (translated to kg./ha), pods per plant, seeds per pod, and seed size were calculated. Adjustments for moisture content were also made to each treatment.

The win rate was defined as the percentage of trials in which one treatment presented higher yields than the control untreated seeds. Four different Extremia A and Extremia MIX Product treatments had a 100% response rate. Extremia A 6 mL had an 85.7% response rate. In the same experiments, the commercial Bradyrhizobium product's response rate was 71.4%.

TABLE 17
Yield evaluation comparison to control treatment

| Yield vs. | Extremia A + | Extremia A | Extremia | Extremia A | Extremia | |
|---|---|---|---|---|---|---|
| Control | Brady | 6 mL | MIX 6 mL | 5 mL | MIX 5 mL | Brady |
| Pergamino | 22% | 15% | 13% | 6% | 6% | 3% |
| Bs As | | | | | | |
| F. Ameghino | 30% | 9% | 17% | 4% | 17% | 6% |
| Bs As | | | | | | |
| San | 21% | 45% | 15% | 40% | 8% | 15% |
| Jerónimo | | | | | | |
| Norte | | | | | | |
| Tucumán | 3% | 3% | 9% | 8% | 1% | −3% |
| Tres Arroyos | 2% | 0% | 1% | 9% | 16% | −4% |
| Bs As | | | | | | |
| Jesus María | 10% | 9% | 7% | 10% | 14% | 6% |
| Entre Rios | 29% | −3% | 6% | 14% | 10% | 3% |
| Average | 16.7% | 11.2% | 9.8% | 13.0% | 10.4% | 3.9% |
| trials (7 | | | | | | |
| treatments) | | | | | | |
| Win rate | 100.0% | 85.7% | 100.0% | 100.0% | 100.0% | 71.4% |

Crop yield data was further analyzed to compare yields of Extremia A and Extremia MIX Products treated seeds versus the commercial Bradyrhizobium product treated seeds. Results are shown in Table 18.

The data showed that Extremia A and Extremia MIX Product treatments generally increased yield for all treatments compared to untreated seeds. Yield levels were much higher for Extremia A and Extremia MIX Product treated seeds than seeds treated with traditional Bradyrhizobium based products. Seeds treated with Extremia A and Extremia MIX Products exhibited 6-12% higher yields than seeds treated with the commercial Bradyrhizobium product. Seeds treated with Extremia A and Extremia MIX Products exhibited higher yields than seeds treated with the commercial Bradyrhizobium products in 6 of 7 trials, or up to the 7 trials in the case of Extremia A+Bradyrhizobium.

The combined analysis of the field trials demonstrated that the Extremia A and Extremia MIX formulation treatments presented significant performance improvements of ˜10-17% versus a control treatment without inoculation, and improvements of ˜6-12% when compared to the commercial Bradyrhizobium based product. The Extremia A and Extremia MIX Product treatments exhibited higher consistency of response rates, between 87-100%, compared to response rates of 71% for the Bradyrhizobium-based product. Fisher's statistical analysis showed that all Extremia A and Extremia MIX Product treatments showed significant differences in performance against the uninoculated control and against the commercial Bradyrhizobium product.

TABLE 18
Yield evaluation comparison to Bradyrhizobium treatment

| Yield vs. | Extremia A + | Extremia A | Extremia MIX | Extremia A | Extremia |
|---|---|---|---|---|---|
| Bradyrhizobium | Brady | 6 mL | 6 mL | 5 mL | MIX 5 mL |
| Pergamino Bs As | 18% | 12% | 10% | 3% | 2% |
| F. Ameghino Bs | 22% | 3% | 10% | −3% | 10% |
| As | | | | | |
| San Jerónimo | 6% | 26% | 0% | 22% | −6% |
| Norte | | | | | |
| Tucumán | 5% | 6% | 12% | 11% | 4% |
| Tres Arroyos Bs | 7% | 5% | 6% | 14% | 22% |
| As | | | | | |
| Jesus María | 4% | 2% | 1% | 4% | 8% |
| Entre Ríos | 25% | −7% | 2% | 10% | 7% |
| Average vs. | 12% | 7% | 6% | 9% | 7% |
| Brady | | | | | |

Results are shown in Table 19.

Yield data comparison with the Bradyrhizobium treatment showed that, on average, the combination of Extremia A exhibited the highest yield improvement. However, this treatment exhibited the top yield in only 3 out of the 7 trials, showing that treatment performance could be affected by the environmental conditions and other factors such as rainfall, existing microbiome, and soil characteristics.

TABLE 19

| | Extremia | | | | | | | Location |
|---|---|---|---|---|---|---|---|---|
| Location/ | A + | Extremia | Extremia | Extremia | Extremia | | | average |
| Treatment | Brady | A 6 ml | MIX 6 ml | A 5 ml | MIX 5 ml | Brady | Control | yield |
| Pergamino | 4272 | 4052 | 3977 | 3723 | 3704 | 3629 | 3508 | 3838 |
| F. | 5292 | 4453 | 4771 | 4223 | 4783 | 4343 | 4079 | 4563 |
| Ameghino | | | | | | | | |
| San | 2900 | 3472 | 2744 | 3347 | 2577 | 2748 | 2388 | 2882 |
| Jeronimo | | | | | | | | |
| Norte | | | | | | | | |
| Tucumán | 3216 | 3220 | 3422 | 3388 | 3166 | 3052 | 3134 | 3228 |
| Tres | 2991 | 2943 | 2971 | 3201 | 3414 | 2805 | 2933 | 3037 |
| Arroyos | | | | | | | | |
| Jesús | 3399 | 3356 | 3309 | 3406 | 3522 | 3275 | 3083 | 3336 |
| Maria | | | | | | | | |
| Entre Ríos | 2390 | 1794 | 1964 | 2112 | 2047 | 1919 | 1856 | 2012 |
| Bengolea | 3199 | | | 3643 | 3562 | | 3098 | 3376 |
| Cnel. | 3950 | | | 3526 | 3756 | | 3369 | 3650 |
| Moldes | | | | | | | | |

### Yield Matrix

At each trial location, four central rows of every experimental unit were harvested, and Yield (kg/ha) was determined in each Experimental unit through measuring its components (Number of pods per plant, N° grains/pod and grain weight).

For the 7 trials, results were compared among treatments to analyze superiority of one treatment over others. Table 9 shows the results from the comparative analysis.

Key conclusions were that all the treatments that included Extremia A or Extremia MIX Products, were superior to Bradyrhizobium in at least ˜86% of the trials. In addition, not enough evidence showed superiority of using 6 ml vs. 5 ml for both Extremia A and Extremia MIX Products, but there was a slight advantage to the lower dosages (better results in 57% of trials). Finally, by calculating the total number of comparisons that were superior for each treatment, it was found that Extremia A 5 ml and Extremia A+Brady exhibited the best results, showing an advantage in 63% of the comparisons.

TABLE 20

| | | | | | | | % |
|---|---|---|---|---|---|---|---|
| Row vs. Column | Extremia | Extremia | Extremia | Extremia | Extremia | | ‘“battles” |
| comparison* | A + Brady | A 6 ml | MIX 6 ml | A 5 ml | MIX 5 ml | Brady | won |
| Extremia A + | | 71% | 43% | 43% | 57% | 100% | 63% |
| Brady | | | | | | | |
| Extremia A 6 ml | 29% | | 43% | 43% | 43% | 86% | 49% |
| Extremia MIX | 14% | 57% | | 43% | 43% | 86% | 49% |
| 6 ml | | | | | | | |
| Extremia A 5 ml | 57% | 57% | 57% | | 57% | 86% | 63% |
| Extremia MIX | 29% | 57% | 57% | 43% | | 86% | 54% |
| 5 ml | | | | | | | |
| Brady | 0% | 14% | 14% | 14% | 14% | | 11% |

*Row is superior to Column in X % of the time

### Example 5: Root Evaluation of Seeds Treated with Extremia a and Extremia MIX

Soybean seeds were inoculated with either Extremia A or Extremia MIX at 5 mL of inoculant per kg of seed or 6 mL of inoculant per kg of seed. Seeds were planted, grown, and harvested in Moldes and Bengolea, in the Province of Córdoba or in Tucuman and San Jeronimo Norte regions as described in Example 4.

Roots of the soybean plants were measured for their length and the number of secondary (lateral) roots. The measurements were carried out 28 days (Moldes) and 29 days (Bengolea) after planting. Results are shown in Table 21 and Table 22. The results indicated that both Extremia A and Extremia Mix have plant growth promoter activity based on the analysis of the development of soybean crop root length and secondary root structure. The crops harvested from Moldes that were treated with Extremia A and Extremia MIX exhibited both an increase in the length of the main root, and an increase in the number of lateral roots. The crops harvested from Bengolea that were treated with Extremia A and Extremia MIX showed an increase in the number of lateral roots.

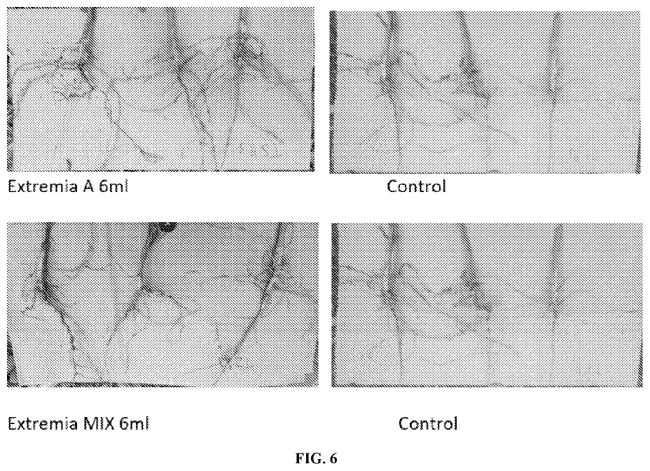

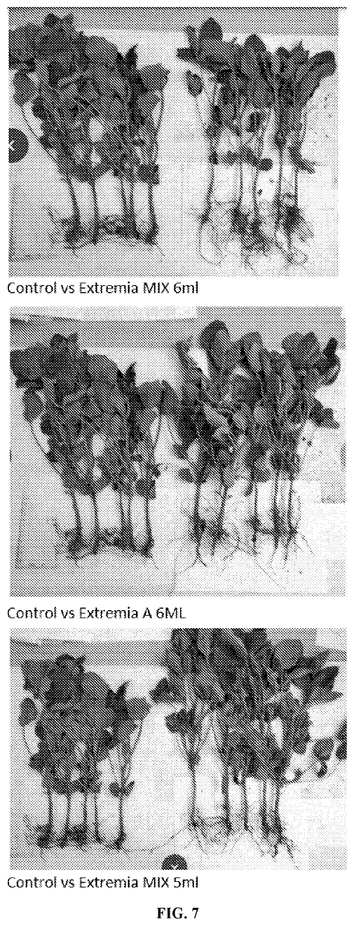

FIG. 6 and FIG. 7 show representative images of crops from the Tucuman and San Jeronimo Norte regions, respectively. As shown, soybean plants treated with Extremia A and Extremia MIX exhibited an increase in root growth development, as well as an increase in biomass.

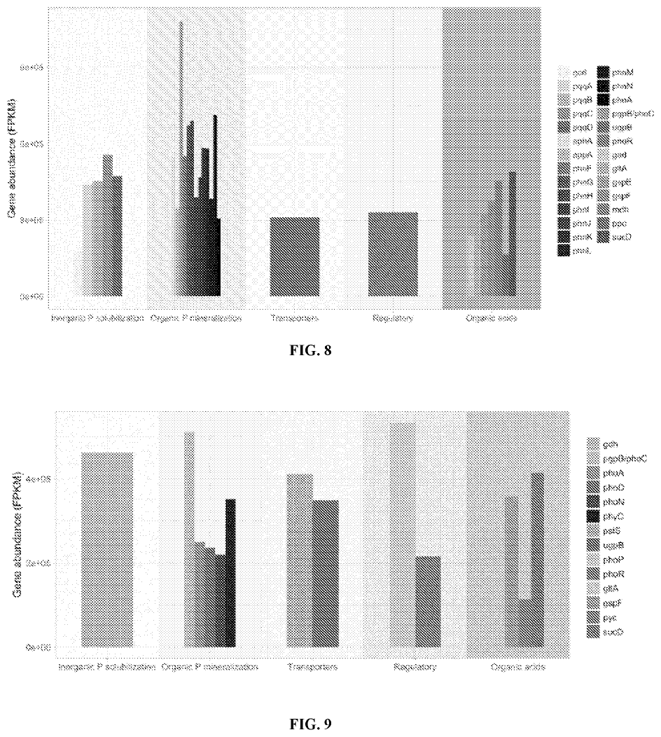

The cufflinks program was used to calculate the fragments per kilobase per million (FPKM) to calculate the abundance of each gene. This quantification is optimal for making comparisons with other genes in the same genome. Considering the length of the gene we can estimate whether gene X is more abundant than gene Y in each genome. However, the comparison between different genomes is not valid. Results of this analysis is found in FIG. 8 and FIG. 9.

TABLE 21
Soybean main root length

| | Length of main roots (cm) | | |
|---|---|---|---|
| Treatments | Bengolea | Moldes | Average |
| Control | 18.4 | 13.9^{B} | 16.2 |
| Extremia A | 18.3 | 19.8^{A} | 19.1 |
| Extremia Mix | 17.9 | 17.7^{AB} | 17.8 |
| ANOVA | NS | 0.0484 | NS |

TABLE 22
Soybean lateral root length

| | Number of lateral roots | | |
|---|---|---|---|
| Treatments | Bengolea | Moldes | Average |
| Control | 44.0^{B} | 35.8 | 39.9^{B} |
| Extremia A | 67.2^{A} | 43.3 | 52.2^{A} |
| Extremia Mix | 66.2^{A} | 37.9 | 52.1^{A} |
| ANOVA | −0.0001 | NS | 0.0001 |

^{A} or ^{B}indicates statistically significant.

### Example 6: Genomic Characterization of CK1 and CK2

The CK1 and CK2 strains were characterized by whole genome sequencing as described in Carriço et al. (Clinical Microbiology and Infection (2018) 24(4):P342-349). The genome assembly analysis for the strains in shown in Table 23. Based on the average nucleotide identity to publicly available species, it was determined that CK1 is most related to Klebsiella and CK2 to Bacillus. Neither strain shared 100% identity to any publicly available genome sequence. An analysis of the genomic sequence for CK1 and CK2 for coding regions, rRNA, repeat regions and tRNA regions is shown in Table 24.

TABLE 23
Genome assembly analysis of CK1 and CK2

| | CK1 | CK2 |
|---|---|---|
| | (Klebsiella aerogenes) | (Bacillus cereus) |
| # contigs (>=0 bp) | 329 | 110 |
| # contigs (>=500 bp) | 40 | 18 |
| # contigs (>=1000 bp) | 26 | 18 |
| Largest contig | 1511170 | 1010096 |
| Total length | 5005304 | 4285098 |
| Total length (>=0) | 5068702 | 4303100 |
| Total length (>=1000) | 4995476 | 4285098 |
| N50 | 537496 | 610525 |
| N75 | 32808 | 516531 |
| L50 | 3 | 3 |
| L75 | 6 | 5 |
| GC (%) | 55.16 | 45.91 |

TABLE 24
Genome annotation analysis of CK1 and CK2

| | CK1 (Klebsiella aerogenes) | CK2 (Bacillus cereus) |
|---|---|---|
| CDS | 4645 | 4415 |
| rRNA | 6 | 8 |
| Repeat region | 0 | 1 |
| tRNA | 77 | 95 |

### Example 7: Nitrogen Fixation Genome Analysis of CK1 and CK2

Using the genomic sequence obtained for CK1 and CK2 in Example 6, the genomes of these strains were analyzed for the presence of N2 metabolic pathways. Paired-end sequences were filtered for quality using the Trimmomatic program and observed through FastQC. The sequences of each strain were filtered. The assembly of the reads was carried out by means of the Spades program. The annotation of the sequence obtained through Spades was made through the Prokka program. Using the annotated genome data, the presence of enzymes corresponding to the nitrogen cycle was searched (N2 fixation, assimilative and dissimilative reduction, denitrification, and nitrification). For this analysis, the FASTA sequences of all the genes of interest related to the metabolic pathway were downloaded from the Uniprot database and only curated sequences were used. With each sequence downloaded, a Psi-blast was performed to obtain a list of 500 homologous sequences that maintained the conserved regions of each sequence and was iterated a minimum of 4 times to obtain sequences as distant as possible.

First, the cdhit program was used to remove redundant sequences. Then, the Clustal program was used to perform a multiple alignment of the 500 sequences corresponding to each enzyme of interest. Finally, multiple alignment was used to obtain a hidden Markov model (HMM) of each enzyme and then this model was used to thin it against the faa file of each annotated genome. In this way, all the sequences present in the genomes that are similar to the enzymatic sequences of interest were searched manually.

Results of genome analysis are shown in Tables 25 and 26, indicating the presence and absence of enzymes for steps involved in nitrogen fixation. The presence and absence of the pathways is summarized in Table 27. Table 28 lists the gene names, enzymes, and protein sequences searched. The results indicated the presence of an incomplete subset of nitrogen fixation pathway enzymes in CK1 and CK2.

TABLE 25
Klebsiella aerogenes CK1 Nitrogen fixation gene signature

| | | | HMM found (E- | | |
|---|---|---|---|---|---|
| Pathway | Gene | EC | value <10⁻³) | Protein name | Present |
| Nitrogen | niKDH | 1.18.6.1 | — | — | No |
| fixation | anfGD | | — | — | |
| | vnfDKG | 1.18.6. | — | — | |
| Assimilatory | nasA; narB | 1.7.7.2 | CIKKEGMN_01222 | Nitrate | Yes |
| nitrate | | 1.7.1.4 | | Reductase | |
| reduction | nasB (igual | | CIKKEGMN_02470 | Nitrite | |
| | que nirB) | | | Reductase | |
| | nirA | 1.7.7.1 | — | — | |
| Dissimilatory | NarG | 1.7.5.1 | CIKKEGMN_01227 | Respiratory | Yes |
| nitrate | | | CIKKEGMN_00439 | nitratereductase | |
| reduction | | | | alpha | |
| (DNR) | narH | | CIKKEGMN_00440 | Respiratory | |
| | | | CIKKEGMN_01228 | nitrate | |
| | | | | reductase beta | |
| | | | | chain | |
| | narI | | CIKKEGMN_01230 | Respiratory | |
| | | | CIKKEGMN_00442 | nitrate | |
| | | | | reductase | |
| | | | | gamma | |
| | napA | 1.9.6.1 | — | — | |
| | nirB (igual | 1.7.1.1.5 | CIKKEGMN_02470 | Nitrite | |
| | que nasB) | | | Reductase large | |
| | | | | subunit | |
| | nirD | | CIKKEGMN_01227 | Respiratory | |
| | | | | nitrate | |
| | | | | reductase | |
| Denitrification | narGHI | 1.7.5.1 | CIKKEGMN_01227 | — | No |
| | (igual que en | 1.9.6.1 | | | |
| | DNR) | | | | |
| | napA (igual | | — | — | |
| | que enDNR) | | | | |
| | nirK | 1.7.2.1 | — | — | |
| | nirS | | — | — | |
| | norB | 1.7.2.5 | — | — | |
| | nosZ | 1.7.2.4 | — | — | |
| Nitrification | amoA | 1.14.99.39 | — | — | No |
| | amoB | | — | — | |
| | Hao1 | 1.7.2.6 | — | — | |

TABLE 26
Bacillus cereus CK2 Nitrogen gene signature

| Pathway | Gene | EC | ck2 ID | Protein name | Present |
|---|---|---|---|---|---|
| Nitrogen | nifKDH | 1.18.6.1 | — | — | No |
| Fixation | anfGD | | — | — | |
| | vnfDKG | 1.18.6. | — | — | |
| Assimilatory | NRT, narK, | | AHCFPGON_00953 | MFS transporter, NNP family, | Yes |
| nitrate | nrtP, nasA | | | nitrate/nitrite transporter | |
| reduction | narGHI (igual | 1.7.99.— | AHCFPGON_00963 | Nitrate reductase | |
| | que en DNR) | | AHCFPGON_00962 | | |
| | | | AHCFPGON_00960 | | |
| | nasE | 1.7.7.4 | AHCFPGON_05730 | Nitrite Reductase small | |
| | | | | subunit | |
| | nasD | | AHCFPGON_05731 | Nitrite Reductase large | |
| | | | | subunit | |
| Dissimilatory | narG | 1.7.5.1 | AHCFPGON_00963 | Respiratory nitrate | Yes |
| nitrate | | | | reductasealpha chain | |
| reduction (DNR) | narH | | AHCFPGON_00962 | Respiratory nitrate | |
| | | | | reductasebeta chain | |
| | narI | | AHCFPGON_00960 | Respiratory nitrate | |
| | | | | reductasegamma chain | |
| | napA | 1.9.6.1 | — | — | |
| | nirB | 1.7.1.15 | AHCFPGON_00946 | Nitrite Reductase large | |
| | | | | subunit | |
| | nirD | | AHCFPGON_00947 | Nitrite Reductase small | |
| | | | | subunit | |
| Denitrification | narGHI (igual | 1.7.5.1 | AHCFPGON_00963 | Respiratory nitrate reductase | No |
| | que en DNR) | 1.9.6.1 | AHCFPGON_00962 | | |
| | | | AHCFPGON_00960 | | |
| | napA (igual | | — | — | |
| | que en DNR) | | | | |
| | nirK | 1.7.2.1 | — | — | |
| | nirS | | — | — | |
| | norB | 1.7.2.5 | — | — | |
| | nosZ | 1.7.2.4 | — | — | |
| Nitrification | amoA | 1.14.99.39 | — | — | No |
| | amoB | | — | — | |
| | hao1 | 1.7.2.6 | — | — | |
| | nxrAB | | AHCFPGON 00963 | Nitrate reductase/nitrite | |
| | | | | oxidoreductase | |

TABLE 27
Summary of Nitrogen Fixation Gene Signature in
Klebsiella aerogenes CK1 and Bacillus cereus CK2

| Pathway | CK1 | CK2 |
|---|---|---|
| N2 fixation | ABSENT | ABSENT |
| Assimilatory nitrate reduction | PRESENT | PRESENT |
| Dissimilatory nitrate reduction (DNR) | PRESENT | PRESENT |
| Denitrification | ABSENT | ABSENT |
| Nitrification | ABSENT | ABSENT |

TABLE 28
Nitrogen Cycle Enzyme Genes

| | | Bacillus cereus | |
|---|---|---|---|
| Enzyme name | Gene name | CK2 ID | FASTA protein sequence |
| MFS transporter, | narK, nasA, | AHCFPGON_00953 | >AHCFPGON_00953 putative nitrate |
| NNP family, | NRT, nrtP | | transporter NarT |
| nitrate/nitrite | | | MKSPNFQLSLQTSNLIIGFMVWVILSS |
| transporter | | | LMPYIKVDIPLTAGQISMVTAVPVIL |
| | | | GSVLRIPIGYWTNRFGARKLFFISFILL |
| | | | LLPVFYISVANSMMDLIIGGLFVGIGG |
| | | | AVFSVGVTSLPKYFPKESHGFVNGIY |
| | | | GVGNAGTAITSFLAPVIATSVGWRTT |
| | | | VQCYLVLLAAFALMNFLLGDRKEKK |
| | | | VNTPLMEQIKGVYKNEKLWFLCIFYF |
| | | | LTFGSFVAFTVYLPNFLVSHFGLEKV |
| | | | DAGMRTAGFIVLATIMRPIGGWLGD |
| | | | KFNPFKILIFVFIGLTLSGIILSFMPSM |
| | | | NVYTFGCLLVAFCAGIGNGTIFKLVP |
| | | | MYFSEQAGIVNGLVSALGGLGGFFPP |
| | | | LILTLLFQLTGHYAIGFMALSEVALA |
| | | | CLIITVWMYSQEKLLVMLKNH |
| Nitrite Reductase | nasD | AHCFPGON_00946 | >AHCFPGON_00946 Nitrite reductase |
| [1.7.7.4] | | | [NAD(P)H] |
| | | | MKKRLVMIGNGMAGIRCMEEILKHD |
| | | | SDSYEITIFGDEPHPNYNRIMLSHVLQ |
| | | | GKTNIQDIIMNEYSWYEENEITLYTN |
| | | | ERVQSINREEKIIITEKKRTLTYDKLII |
| | | | ATGSSAFILPVEGSALSGVTGFRTIED |
| | | | TQFMIDTAKEKKKAVVIGGGLLGLE |
| | | | AARGLIDLGMDVHVVHLMPSLMEQ |
| | | | QLDTKAAALLREDLEAQGMKFLME |
| | | | KKTVKILGTDHVEGIQFEDGEVVDC |
| | | | DLIVMAVGIRPNTQIAKDAGLIVNRG |
| | | | IVVNDYMLTNDESIYAVGECAEHDGI |
| | | | AYGLVAPLYEQGAILAKHITNLQTD |
| | | | GYSGSIVGTQLKVAGCDLFSAGQIYE |
| | | | DDQTKAISIFNECKRSYKKILIRDNKV |
| | | | VGIVLYGDTADGTRLFSILKKEEDIQ |
| | | | EYTPASILHKAGEECELDVATMSAD |
| | | | DTICGCNGVTKGTIVHAILEQELKTF |
| | | | EEVKACTKAAGSCGKCRPLVEQVLS |
| | | | HTLGDAFDASAQSTGMCGCTPLSRD |
| | | | EVVAAIHEKGLKSPKEVRNVLGFVH |
| | | | EDGCSKCRPALNYYLRMAIPEEYED |
| | | | DKSSRFVNERMNGNIQHDGTFSVIPR |
| | | | MYGGVTTADDLMKIAEVAKKYDVP |
| | | | LVKITGASRIGLYGVKKQDLPNVWA |
| | | | ELNMASGYAYSKSLRNVKSCVGSRF |
| | | | CRFGTKDSLGLGMLLEQSLEMVDTP |
| | | | HKMKMGVTGCPRNCAEVLTKDFGV |
| | | | VCVENGYQLYIGGNGGTEVREADFV |
| | | | IIVPTEDDVLRIATAYMQYYRETGIY |
| | nasE, nirD | AHCFPGON_00947 | GERTAYWTERLGFDHIKEILQDANM |
| | | | VTKLNERFQKARGTYKEAWGQALE |
| | | | TKSLKAMYEVETVK |
| | | | >AHCFPGON_00947 Assimilatory nitrite |
| | | | reductase [NAD(P)H] small subunit |
| | | | MIQTKEKIKVMRAEDLPIQIGKEVQM |
| | | | KGMSFALFRLSNGDIRAVENRCPHK |
| | | | KGPLAEGIVSGEFVFCPLHDWKISLL |
| | | | TGEVQKPDDGCIQTYEVEVIDGDIYI |
| | | | YM |
| Nitrite Reductase | nasD | AHCFPGON_05731 | >AHCFPGON_05731 Nitrite reductase |
| [1.7.7.4] | | | [NAD(P)H] |
| | | | MCGCTPLSRDEVIAAIHEKGLKSPKE |
| | | | VRNVLGFAHEDGCSKCRPALNYYLR |
| | | | MTIPEEYEDDKSSRFVNERMNGNIQH |
| | | | DGTFSVIPRMYGGVTTADDLMKIAE |
| | | | FAKKYDVPLVKITGASRIGLYGVKK |
| | | | QDLPNVWAELNMTSGYAYSKSLRN |
| | | | VKSCVGSRFCRFGTKDSLGLGMLLE |
| | | | QSLEMVDTPHKIKMGVTGCPRNCAE |
| | | | VLTKDFGIVCVENGYQLYIGGNGGT |
| | | | EVREADFVMIVPTEDDVLRIATAYM |
| | | | QYYRETGIYGERTAYWTERLGFDHI |
| | | | KEILQDANMVTKLNERFQTARGTYK |
| | | | EAWGQALETKSLKAMYGVETVK |
| | nasE, nirD | AHCFPGON_05730 | >AHCFPGON_05730 Assimilatory nitrite |
| | | | reductase [NAD(P)H] small subunit |
| | | | MLQSKEKFKIMRAEDLPFQIGKEVQ |
| | | | MKGISIILFRLLNGDIRAVENHCPHKN |
| | | | GPLAEGIVSGEFVFFPLHDWKISLVT |
| | | | GEVQKPDDGCIQTYEVEVIDGDIYIY |
| | | | M |
| nitrate reductase/ | narG, narZ, | AHCFPGON_00963 | >AHCFPGON_00963 Respiratory nitrate |
| nitrite | nxrA | | reductase 1 alpha chain |
| oxidoreductase | | | MKKKTSALMRRLKYFSPIDRYNDNH |
| [EC:1.7.5.1 | | | TQETYEDREWENVYRKRWQHDKVI |
| 1.7.99.-] | | | RSTHGVNCTGSCSWNIYVKDGIVTW |
| | | | EGQELNYPTTGPDMPDFEPRGCPRG |
| | | | ASFSWYIYSPLRVKYPYVRGVLWNM |
| | | | WQEELQNNESPLEAWKSIVENREKA |
| | | | RTYKQARGKGGFIRVNWDEVLQLVS |
| | | | ASLLYTVMKYGPDRNVGFSPIPAMS |
| | | | MLSHAAGSRFMQLMGGPMLSFYDW |
| | | | YADLPPASPQIWGDQTDVPESSDWY |
| | | | NSGYIMTWGSNVPMTRTPDAHFLAE |
| | | | VRYKGTKVVSVSPDFAESTKFADDW |
| | | | ISVKQGTDGALAMAMGHVILQEFYV |
| | | | DNQVEYFTKYVKQYTDFPFFVTLKQ |
| | | | KGDQFVADRFLNASDIGRETKLGEW |
| | | | KPVLWNENTNDFATPHGTMGSRWD |
| | | | NEKKWNLRLEDEETGEKIDPRLSLLG |
| | | | MEDSVQTVQIPYFSDDGNKILERTIP |
| | | | VKKVMTEEGELFVTTVYDLTLANYG |
| | | | VNRGVGGQEPKDFNDDIPFTPAWQE |
| | | | KMTGVKRELIIQIAREFAQNAVDING |
| | | | RSMIIVGAGINHWFNSDTIYRAVLNL |
| | | | VLLVGAQGVNGGGWAHYVGQEKL |
| | | | RPAEGWQTIAMAKDWQGPPKLQNG |
| | | | TSFFYFVTDQWRYEDTPVGHLASPV |
| | | | EGNSRYQHHGDYNVLAARLGWLPS |
| | | | YPTFEKNGIELYKEAVAAGATTQEEI |
| | | | GKYVAQKLKEKELKFAIEDPDNKNN |
| | | | FPRNLFVWRANLISSSGKGHEYFLKH |
| | | | LLGTTNGLMNDDSDSLRPEEIKWHE |
| | | | EAPEGKLDLLINLDFRMAGTALYSDI |
| | | | VLPASTWYEKHDLSSTDMHPFVHPF |
| | | | NPAIGSPWEARSDWDIFTSLSKAVSD |
| | | | LAKKIDLEPMKEVVATPLLHDTPQEL |
| | | | AQPLGKIKDWSKGECEPIPGKTMPQI |
| | | | HVVERDYKTIYDKMTALGPNAGKQP |
| | | | IGTKGISWSAEKEYEQLKSKLGVVRT |
| | | | DSIAKGCPDIKEAINAAEAVLTLSSTT |
| | | | NGHMAVKAWEALEKQTDLKLRDLA |
| | | | EEREEECFTFEQITAQPKTVITSPAFT |
| | | | GSEKGGRRYSPFTTNVERLIPWRTIT |
| | | | GRQSFYLDHDMMKEFGETMATFKPI |
| | | | LQHKPFRKSRPEVEGKEITLNYLTPH |
| | | | NKWSIHSMYFDSLPMLTLFRGGPTV |
| | | | WMNKEDAAEAGVADNDWIECFNRN |
| | | | GVVVARAVVTHRIPRGMAFMHHAQ |
| | | | DRHINVPGTKLTSNRGGTHNSPTRIH |
| | | | VKPTHMIGGYGQLSYGFNYYGPTGN |
| | | | QRDLNVVIRKLKEVDWLED |
| | narY, narH, | AHCFPGON_00962 | >AHCFPGON_00962 Respiratory nitrate |
| | nxrB | | reductase 2 beta chain |
| | | | MKIKAQVGMVMNLDKCIGCHTCSV |
| | | | TCKNTWTNRPGAEYMYFNNVETKP |
| | | | GIGYPKQWEDQEKYKGGWELKNGEI |
| | | | QLKSGSKMKRLMNIFHNPDQPTIDD |
| | | | YFEPWNYDYETLTNSPQRKHQPVAR |
| | | | PKSAITGEFIDKIEWGPNWEDDLAGG |
| | | | HITGLQDPNVKKMEEEIKTDFENVF |
| | | | MMYLPRICEHCMNPSCVSSCPSGAM |
| | | | YKREEDGIVLVDQNACRAWRFCVSS |
| | | | CPYKKVYFNWQTNKAEKCTMCFPRI |
| | | | EAGMPTICSETCVGRIRYIGVMLYDA |
| | | | DKVKEAASVEDEKDLYESQLTVFLD |
| | | | PNDPEIAAEAKKQGIPEEWIKAAQQS |
| | | | PIYKMIIDWKIALPLHPEYRTMPMVW |
| | | | YIPPLSPIMNMVEGKGSNWQAEEVFP |
| | | | AIDNMRIPIQYLANLLTAGDESHIRLT |
| | | | LKKMAVMRTYMRALQINKEPNEAV |
| | | | LKELGLAKQDVEDMYRLLAIAKYKD |
| | | | RFVIPTSHREQVADLYSEQGSCGLSF |
| | | | TGGPGSCMTIS |
| | narJ, narW | AHCFPGON_00961 | >AHCFPGON_00961 Nitrate reductase- |
| | | | like protein NarX |
| | | | MRQSLQTAFSCSSFLLSYPELGWREA |
| | | | VTELQEEVETIKQEDVKASLTAFIKQ |
| | | | VLNKTNDQLIDSYVYTFDFGKKTNM |
| | | | YLTYMNTGEQRERGIELLELKQHYK |
| | | | KSGFEVTDKELPDYLPLLLEFFANAN |
| | | | EIDSEPIMSKYTENIQALHVQLKEAD |
| | | | SMYEPILAAVLLAIETWGVQTN |
| | narI, narV | AHCFPGON_00960 | >AHCFPGON_00960 Nitrate reductase- |
| | | | like protein NarX |
| | | | MMDQFLWVLFPYIIFAIFIGGHIFRYN |
| | | | YDQFGWTSKSSELLEKKMLRVGSLL |
| | | | FHFGIMFVIGGHVMGILIPEAVYRSIG |
| | | | ISEHMYHVVAISFGLPAGVASIIGLIIL |
| | | | TYRRVTVKRIIATSTKGDYIALILLLI |
| | | | VMLAGLSSTFLNIDSKGFDYRTTIGP |
| | | | WFRSLFIFQPKVEYMMEVPIWFKIHI |
| | | | LAGMGLFAVWPFTRLVHVFSAPIKY |
| | | | VSRSYVIYRRRIPNELKK |
| nitric-oxide | nos | AHCFPGON_04505 | >AHCFPGON_04505 Nitric oxide |
| synthase, bacterial | | | synthase oxygenase |
| [EC:1.14.14.47] | | | MSKTKQLIEEASNFITICYKELHKEQL |
| | | | IEERIKEIQIEIEKTGTYEHTFEELVHG |
| | | | SRMAWRNSNRCIGRLFWSKMHILDA |
| | | | REVNDEEGVYNALIHHIKYATNDGK |
| | | | VKPTITIFKQYQGEENNIRIYNHQLIR |
| | | | YAGYKTETGVIGDSHSATFTDFCQEL |
| | | | GWQGEGTNYDVLPLVFSIDGKAPIY |
| | | | KEIPREEVKEVPIEHPEYPISSLGVKW |
| | | | YGVPMISDMRLEIGGISYTAAPFNGW |
| | | | YMGTEIGARNLADHDRYNLLPAVAE |
| | | | MMDLDTSRNGTLWKDKALIELNIAV |
| | | | LHSFKKQGVSIVDHHTAAQQFQQFE |
| | | | KQEAACGRVVTGNWVWLIPPLSPAT |
| | | | THIYHKPYPNEILKPNFFHK |

TABLE 46
Bacillus cereus genes involved in plant growth promoting features

| Enzyme | Gene | |
|---|---|---|
| name | name | FASTA protein sequence |
| nitric-oxide | nos | >AHCFPGON_04505 Nitric oxide synthase oxygenase |
| synthase, | | MSKTKQLIEEASNFITICYKELHKEQLIEERIKEIQIEIEKTGTYEHTFEE |
| bacterial | | LVHGSRMAWRNSNRCIGRLFWSKMHILDAREVNDEEGVYNALIHHI |
| [EC: 1.14.14.47] | | KYATNDGKVKPTITIFKQYQGEENNIRIYNHQLIRYAGYKTETGVIGD |
| | | SHSATFTDFCQELGWQGEGTNYDVLPLVFSIDGKAPIYKEIPREEVKE |
| | | VPIEHPEYPISSLGVKWYGVPMISDMRLEIGGISYTAAPFNGWYMGT |
| | | EIGARNLADHDRYNLLPAVAEMMDLDTSRNGTLWKDKALIELNIAV |
| | | LHSFKKQGVSIVDHHTAAQQFQQFEKQEAACGRVVTGNWVWLIPPL |
| | | SPATTHIYHKPYPNEILKPNFFHK |
| salicylate | pchA | >AHCFPGON_00781 Isochorismate synthase DhbC |
| biosynthesis | | MNEHIAVKELSEKLLEDYKTESSFFFASPTRTILAEGEFTTVKHREIES |
| isochorismate | | FPELVQAKLSNAKQAGNPNPIVVGALPFDRRKEVQLIVPEYSRISERL |
| synthase | | QLDTTNQLETNENVTFEMTPVPDPEVYMNGVKQGIEKIQDGDLKKI |
| [EC:5.4.4.2] | | VLSRSLDVKSSEKIDKQKLLRELAEHNKHGYTFAVNLPKDEKENSKT |
| | | LIGASPELLVSRNGMQVISNPLAGSRPRSEDPVEDKRRAEELLSSPKD |
| | | LHEHAVVVEAVAAALRPYCHTLHVPEKPSVIHSEAMWHLSTEVKGE |
| | | LKDPNTSSLQLAIALHPTPAVCGTPMEKAREAIQHIEPFDREFFTGML |
| | | GWSDLNGDGEWIVTIRCAEVQENTLRLYAGAGVVAESKPEDELAET |
| | | SAKFQTMLKALGLRDSSLNEK |
| salicylate | pchA | >AHCFPGON_02841 Salicylate biosynthesis isochorismate synthase |
| biosynthesis | | MIQTKQKGLQEVLSAAIKHATDEKILVSFVKQIDWMDPLLFYAAGK |
| isochorismate | | RIALENRCYFADPAQHVIFAGIGSVFTIANSSHKRFQAARDEWDKVK |
| synthase | | EKAFVQREKYEFGTGPLLFGGFSFDQEKEKTDLWKEFDDTTFSLPAF |
| [EC:5.4.4.2] | | LLTVKNEKAWLTMNTFVSATDCAETLYNEIVSLEEKIFGESKCALEG |
| | | SKLTVTSKVEVDPKGWMKAIEKVQDEMKQGNVQKVVLARELKVE |
| | | MDHHIDSALVLEALRIGQPDCYVFSFDYKGACFLGATPERLIRKEDE |
| | | KFTSMCLAGSTGHGQSIEESKRNSNALLHDEKNLAEHGYVVNMIRS |
| | | VLNEHCEYVNIPESPGLLTTKNLIHLYTPVEAKGTASLLTMVEELHPT |
| | | PALGGTPRLEAMKLIRDVELLDRGLYGAPIGWIDDEGNGEFAVALRC |
| | | GLLNGEKASLFAGCGIVIDSVPQLEYEETSLKFRPMLGALEELMK |
| isochorismate | pchB | >AHCFPGON_00157 Protein AroA(G) |
| pyruvate lyase | | MANHELDQLRKQVDEINLQLLHLLNKRGEIVQKIGEQKQVQGTKRF |
| [EC:4.2.99.21] | | DPVREREVLDMIAEHNEGPFETSTVQHIFKTIFKASLELQEDDNRKAL |
| | | LVSRKKKQENTIVDVKGELIGNGTQTFIMGPCAVESLEQVRQVGQA |
| | | MKDQGLKLMRGGAFKPRTSPYDFQGLGVEGLQILRQVADEFDLAIIS |
| | | EILNPNDVEMALDYVDVIQVGARNMQNFDLLRAVGKVNKPVLLKR |
| | | GLAATIDEFINAAEYIIAQGNDQIILCERGIRTYERATRNTLDISAVPIL |
| | | KKETHLPVIVDVTHSTGRRDLLLPTAKAALAIGADAVMAEVHPDPA |
| | | VALSDSAQQMDIPEFHRFMDELKGFKNKLS |
| isochorismate | pchB | >AHCFPGON_03019 Protein AroA(G) |
| pyruvate lyase | | MASQQLGRLRSEIDQLNLQILELLNERGRLVQEVGNLKEVQGVKRF |
| [EC:4.2.99.21] | | DPVRERNMLDLIAENNNGPFETSTLQHIFKQIFQAGLELQEDDHRKA |
| | | LLVSRKKKTEDTIVEINGEKIGDGNQHFIMGPCAVESYEQVRQVAEA |
| | | MKEQGLKLMRGGAFKPRTSPYDFQGLGLEGLQILRQVADEYDLAVI |
| | | SEILNPNDIEMSLDYVDVIQIGARNMQNFDLLRAAGAVNKPVLLKRG |
| | | LSATIEEFINAAEYIMAKGNGNIILCERGIRTYERATRNTLDISAVPILK |
| | | KETHLPVVVDVTHSTGRRDLLLPTAKAAMAIGADAIMAEVHPDPAV |
| | | ALSDVAQQMNIPQFNDFMNELKSFGSKL |
| pyochelin | pchC | >AHCFPGON_00778 Dimodular nonribosomal peptide synthase |
| biosynthetic | | MPNSQKIRHSLSSAQSGMWFAQQLDPLNPIYNTGEYVEINGNIHQEIF |
| protein PchC | | ELAVRKVVIEAEALHVRFEEDEIGPWQVIEESQFHMHFIDVRKEENPE |
| | | EAAKVWMKNDLSMPVDLKKDTLFTEALIQVENNRFFWYQRIHHIV |
| | | MDGYGFSLLSQKVANEYTSLIEETNKNEKPFGSLTKVVQEDIEYRDS |
| | | KKFQEDRTFWLEKFADEPEVVSLAERAPRTSNGFSRETAYLSSSSTK |
| | | TLLEDINISLTSWPEFIVAVTSIYMHKLTGANDIVLGLPMMGRLGSVS |
| | | IHTPSMVMNLVPLRITVTPNITLAELLQQVSKEIRDVRRHYKYRHEEL |
| | | RRDLKLLGENQRLFGPLVNVMPFDYGLNFAGNRGITHNLSAGPVDD |
| | | LSINVYKRFDQNKLMIHFDANPEVYNGAELALHKERFMSLFELVVN |
| | | NYEKNESIGKINITLPEENHKVLLEWNETKEDDELLSLPISFEKQVQK |
| | | NPNKLAITCDGVNLTYKELNARANELAHYLVEEGIRPNQFVALVFPR |
| | | SIEMVVSMLAVLKAGAAYLPIDPEYPAERINYIVNDAKPVCIITHSSV |
| | | SSKLVIENDMKKIVLDEEETKLALHTYSRMNIACKNDVSLLNPAYTI |
| | | YTSGSTGNPKGVIVPMRGLSNFLMAMQQKFSLNENDHLLAVTTFAF |
| | | DISALEIYLPLISGASLTIAQKEDIQEPSALTTLLQEERVTIMQATPTLW |
| | | QALVTDYPEKLQGLNILVGGEALPEHLANKLKELGCSITNLYGPTET |
| | | TIWSTFMNIDEGEKGIPPIGKPICNTEVYVLDAGLQPVPPGVIGELYIA |
| | | GEGLASGYLGKPELTAERFIANPYGESGKRMYRTGDLVKWRSDGAL |
| | | EYISRADHQIKIRGFRIELAEIETVLQRHENIQQAVVIVREDRPNDKRII |
| | | AYIVAEEKEPINLSEIRSYVSESLANYMIPSAFVVLEELPLTPNGKVDR |
| | | KKLPAPDFNRMDNERVARNPKEEILCDLFAEVLGVSRISIDDNFFEM |
| | | GGHSLLASRLMARIRETLSVELGIGKLFESPTVAELAKQLNHAKSAR |
| | | PAIQKASRPNEVPLSFAQRRLWFLNCLEGPSPTYNIPLVIRMNGILNR |
| | | EALQGAFYDVVEKHETLRTIFPNVLGSSYQRILDMENLNLEMVITNT |
| | | CKDELESVFSEAVRYSFNLDFEPAVRLQLFTVSENEHVLLILLHHIVG |
| | | DGWSLQPLTRDFTAAYKARCQGDRVQLETLSVQYADYALWQQQLL |
| | | GDETTPESLISTQLDFWKEELKGLPDQMELPTDFQRPIETSYRGETIHF |
| | | HIDEGMHSRLVELARKNGVSLFMVLQAGLSALFTRLGAGTDIPIGSPI |
| | | AGRNDDVLSDIVGLFVNTLVLRTNTSGDPSFKELLNRVKQVNLAAY |
| | | ENQDVPFERLVEVLNPVRTRNSHPLFQVMLAFQNTPEAIFDAPDIEAS |
| | | LEIQSVGSAKFDLTFEISESNGVDGTPNGLHGLLEFSTDLYKRETVQK |
| | | LIERFILLLDDAATNPDQSIGRLEILTVAEKNTVLEKWNGGFQIAPEM |
| | | TLPQLFEKQVHINPNSIAVVFEDKKLTYEELNRKANKIARFLIAKGIG |
| | | PDQLVALAMPRSLNMVVSLLAVLKAGAGYLPLDPDYPADRISFMLH |
| | | DAKPTCVLTNSEVEIECNEALKVLVDDVKVIAEVEKYSEENIDEVERI |
| | | KPLSPSHIAYVIYTSGSTGRPKGVMIPHQNVVRLLGATDHWFQFDGN |
| | | DVWTMFHSYAFDFSVWEIWGPLLYGGRLVVVPHTVSRSPKEFLQLL |
| | | VKEKVTVLNQTPSAFYQLMQADRENEEIGQKLSLRYVVFGGEALEL |
| | | SRLEDWYSRHPHNAPKLINMYGITETTVHVSYIELDETIVSLRANSLI |
| | | GCSIPDLKVYVLDNYLQPVPPGVVGEMYVAGAGLARGYLGRAGLT |
| | | AERFIADPFGKPGTRMYRTGDLARWRKDGTLDYIGRADHQIKIRGFR |
| | | IELGEIEAVIMKHPKVEQVVVIVREDQPGDKRLVSYIVASNNEAIDTN |
| | | EMRQFASGSLPDYMVPYDFVVVNELPLTPNGKLDRKALPAPEFIASS |
| | | SSRGPRTPQEEMLCDLFTEVLSVPQIGIDDGFFDLGGHSLLAVQLMSR |
| | | MKEALGVELNIGTLFAAPTVAGLAERLEMGNGQSALDVLLSLRASG |
| | | DQLPLFCVHPAGGLSWCYAGLMKSLGTDYPIYGVQARGIAKNEELP |
| | | KSLEEMAADYLKQVREVQPHGPYRLLGWSLGGNVVHAMAAQLQN |
| | | EGEEVELLVMLDSYPGHFLPNTEAPTEEEALIALLALGGYDPDNMDG |
| | | KPLTMESAVEILRKDGSALASLEEETILNLKETYVNSVGLLGKYVPK |
| | | VYNGDILFFRSTVIPDWFDPISPNTWLNYLDGQIVQHDIDCRHKDLC |
| | | QPGPLTEIGQVLAKYLQNKKGVSRV |
| pyochelin | pchD | >AHCFPGON_00780 2,3-dihydroxybenzoate-AMP ligase |
| biosynthesis | | MLVGYTEWPKEFADRYREAGCWLGETFGGVLRERAEKYGDRIAVV |
| protein PchD | | SGKKHITYSELNKKVDRLAAGLLNLGIKKEDRVVIQLPNIIEFFEICFA |
| | | LFRIGALPVFALPSHRSSEISYFCEFGEASAYVISDKALGFDYRKLARE |
| | | VKEKVPTLQHVIVVGEEEEFVNINDLYMDPVSLPEVQPSDVAFLQLS |
| | | GGTTGLSKLIPRTHDDYIYSLRVSAEICNLNAESVYMAVLPVAHNYP |
| | | MSSPGTFGTFYAGGKVVLATGGSPDEAFALIEKEKVTITALVPPLAMI |
| | | WLDAASSRNADLSSLEVIQVGGAKFSAEVAKRIRPTFGCTLQQVFG |
| | | MAEGLVNYTRLDDPEEFIIYTQGRPMSALDEVRVVDENDNDVQPGE |
| | | VGSLLTRGPYTIRGYYKAEEHNARSFTKDGFYRTGDLVKVNEQGYII |
| | | VEGRDKDQINRGGEKVAAEEVENHLLAHDSVHDVAIVSMPDDYLG |
| | | ERTCAFVIARGQVPAVSELKMFLRERGIAAYKIPDRIEFIEAFPQTGV |
| | | GKVSKKELRKVIAEKLITVKQ |
| dihydro- | pchE | >AHCFPGON_00779 Isochorismatase |
| aeruginoic acid | | MAIPSISVYKMPIESELPKNKVNWTPDPKRAVLLIHDMQEYFLDAYS |
| synthetase | | DKESPKVELISNIKMIREKCKELGIPVVYTAQPGGQTLEQRGLLQDF |
| | | WGDGIPAGPDKKKIVDELTPDEDDIFLTKWRYSAFKKTNLLEILNEQ |
| | | GKDQLIICGIYAHIGCLLTACEAFMDGIEPFFVADAVADFSLEHHKQA |
| | | LEYASNRCAVTTSTNLLLNDLQSVKDDESEGITIQEVHELVAQLLREP |
| | | VESIDIDEDLLNRGLDSVRIMSLVEKWRREGKEITFAHLAERPTVAG |
| | | WYSLLSSQTAQVL |
| tryptophan | trpA | >AHCFPGON_02664 Tryptophan synthase alpha chain |
| synthase | | MGVEKIKAAFENGKKAFIPYVMGGDGGFEKLKERIRFLDEAGASIVE |
| [EC:4.2.1.20] | | IGIPFSDPVADGPTIQRAGKRALDSGVTVKGIFQALIEVRKEVQIPFVL |
| | | MTYLNPVLAFGKERFIENCIEAGVDGIIVPDLPYEEQDIIAPLLREANV |
| | | ALIPLVTITSPIERIEKITSESKGFVYAVTVAGVTGVRQNFKEEIHSYLE |
| | | KVKSHVNLPVVAGFGISTKEHVEEMVTICDGVVVGSKIIELLENEKQ |
| | | EEICELIYATKQKEEA |
| | trpB | >AHCFPGON_02665 Tryptophan synthase beta chain |
| | | MNYAYPDEKGHYGIYGGRYVPETLMQSVLELEEAYKEAMQDEAFQ |
| | | KELNHYLKTYVGRETPLYFAENLTKYCGGAKIYLKREDLNHTGAHK |
| | | INNTIGQALLAVRMGKKKVVAETGAGQHGVATATVCALLGLECVIF |
| | | MGEEDVRRQKLNVFRMELLGAKVESVAAGSGTLKDAVNEALRYW |
| | | VSHVHDTHYIMGSVLGPHPFPQIVRDFQSVIGNETKKQYEELEGKLP |
| | | EAVVACIGGGSNAMGMFYPFVHDEEVALYGVEAAGKGVHTEKHA |
| | | ATLTKGSVGVLHGSMMYLLQNEEGQIQEAHSISAGLDYPGVGPEHS |
| | | LLKDIGRVSYQSITDEEALEAFQLLTKKEGIIPALESSHAVAYALKLA |
| | | PKMKEDEGLVICLSGRGDKDVESIKRYMEEV |
| indole-3- | trpC | >AHCFPGON_02667 Indole-3-glycerol phosphate synthase |
| glycerol | | MGTILDKIVEQKKKEVAELYEIYTPVKAKRKAHSLVEALQQFTVIAE |
| phosphate | | VKRASPSKGDINLHVDVRKQVGTYEKCGAGAVSVLTDGQFFKGSFH |
| synthase | | DLQTAREESNIPLLCKDFIIDKIQIDRAYEAGADIILLIVAALTKEKLKE |
| [EC:4.1.1.48] | | LYSYVLEKGLEAIVEVHDEQELETAIVLNPHVIGINNRNLKTFEVDLS |
| | | QTEKLGKRLNEEKLLWISESGIHSKEDIIRVKRAGAKGVLVGEALMT |
| | | SSSISSFFEDCKVNI |
| anthranilate | trpD | >AHCFPGON_02668 Anthranilate phosphoribosyltransferase 2 |
| phosphoribosy | | MNNYLRKLVEGQHLTEEEMYKAGLLLLSENILESEIAAFLVLLKAKG |
| ltransferase | | ETAEEIYGLVRALREKALPFSNHIQGAMDNCGTGGDGAQTFNISTTS |
| [EC:2.4.2.18] | | AFVLAGAGVKVAKHGNRAVSSKTGSADLLEELGVNISSTPNEIDYLL |
| | | EHVGIAFLFAPAMHPALRRIMKIRKELNVPTIFNLIGPLTNPVNLETQF |
| | | VGIYKRDMLLPVAQVLQKLGRKQALVVNGSGFLDEASLQGENHVV |
| | | LLKDNEIVEMSIDPEKYGFSRVKNEEIRGGNSKENAKITLEVLSGEKS |
| | | VYRDTVLLNAGLALFANGKTETIEEGIKLAAHSIDSGKALTKLNLLIA |
| | | ASNEKLERVN |
| indole-3- | trpC | >>AHCFPGON_02667 Indole-3-glycerol phosphate synthase |
| glycerol | | MGTILDKIVEQKKKEVAELYEIYTPVKAKRKAHSLVEALQQFTVIAE |
| phosphate | | VKRASPSKGDINLHVDVRKQVGTYEKCGAGAVSVLTDGQFFKGSFH |
| synthase | | DLQTAREESNIPLLCKDFIIDKIQIDRAYEAGADIILLIVAALTKEKLKE |
| [EC:4.1.1.48] | | LYSYVLEKGLEAIVEVHDEQELETAIVLNPHVIGINNRNLKTFEVDLS |
| | | QTEKLGKRLNEEKLLWISESGIHSKEDIIRVKRAGAKGVLVGEALMT |
| | | SSSISSFFEDCKVNI |
| anthranilate | trpD | >AHCFPGON_02668 Anthranilate phosphoribosyltransferase 2 |
| phosphoribosy | | MNNYLRKLVEGQHLTEEEMYKAGLLLLSENILESEIAAFLVLLKAKG |
| ltransferase | | ETAEEIYGLVRALREKALPFSNHIQGAMDNCGTGGDGAQTFNISTTS |
| [EC:2.4.2.18] | | AFVLAGAGVKVAKHGNRAVSSKTGSADLLEELGVNISSTPNEIDYLL |
| | | EHVGIAFLFAPAMHPALRRIMKIRKELNVPTIFNLIGPLTNPVNLETQF |
| | | VGIYKRDMLLPVAQVLQKLGRKQALVVNGSGFLDEASLQGENHVV |
| | | LLKDNEIVEMSIDPEKYGFSRVKNEEIRGGNSKENAKITLEVLSGEKS |
| | | VYRDTVLLNAGLALFANGKTETIEEGIKLAAHSIDSGKALTKLNLLIA |
| | | ASNEKLERVN |
| anthranilate | trpE | >AHCFPGON_02670 Anthranilate synthase component 1 |
| synthase | | MMTKEEFIKQKRERKTFLVITEEEGDSITPISLYRRMKGKKKFLLESS |
| component I | | QLHQDKGRYSYLGCNPYGEVKSVGTEVERTIFGRAEKLQGNVLQVL |
| [EC:4.1.3.27] | | EEVIAPSQVDSPFPFCGGAVGYIGYDVIRQYENIGADLHDPLNIPEVH |
| | | LLLYREFIVYDHLRQKLSFVYVCREDDSTDYEEVYERLRVYKEEVLQ |
| | | GEEAEVNAIQSPLSFTSSITEKEFCEMVEIAKEYIRAGDIFQVVLSQRL |
| | | QSECIGDPFALYRKLRIANPSPYMFYIDFQDYVVLGSSPESLLSVRED |
| | | KVMTNPIAGTRPRGKTKREDEEIAKELLGNEKERAEHMMLVDLGRN |
| | | DIGRVSEIGSVTIDKYMKVEKYSHVMHIVSEVYGTLRKQMGGFDAL |
| | | AYCLPAGTVSGAPKIRAMEIINELENEKRNVYAGAVGYVSFSGNLD |
| | | MALAIRTMVVKDEKAYVQAGAGVVYDSDPVAEYEETLNKARALLE |
| | | VMK |
| phosphoribosy | trpF | >AHCFPGON_02666 N-(5′-phosphoribosyl)anthranilate isomerase |
| lanthranilate | | MKVKICGITDMETAKSACEYGADALGFVFAESKRKITPKRAKEIIQEL |
| isomerase | | PANVLKIGVFVNESVEVIQKIADECGLTHVQLHGDEDNYQIRRLNIPS |
| [EC:5.3.1.24] | | IKSLGVTSESDMKNAQGYETDYILFDSPKEKFHGGNGKTFSWELLGH |
| | | MPKELRKKTILAGGLNALNIEEAIRTVRPYMVDVSSGVETEGKKDVE |
| | | KIKQFIIKAKECSK |
| indoleacetamide | gatA, iaaH | >AHCFPGON_03992 Glutamyl-tRNA(Gln) amidotransferase subunit A |
| hydrolase | | MKKWVKVTLSITGGIVLLACAGGYYVYKNYFPKESERIVYDKERVL |
| [EC:3.5.1.-] | | QPIHNQLKGINIENVKIKEKEVVNATVDELQKMIDDGKLSYEELTSIY |
| | | LFRIQEHDQNGITLNSVTEINPNAMEEARKLDQERGRNKNSNLYGIP |
| | | VVVKDNVQTEKVMPTSAGTYVLKDWIADQDATIVKQLKEEGAFVL |
| | | GKANMSEWANYLSFTMPSGYSGKKGQNLNPYGPITFDTSGSSSGSA |
| | | TVVAADFAPLAIGTETTGSIVAPAAQQSVVGLRPSLGMVSRTGIIPLV |
| | | ETLDTAGPMARTVKDAATLFNAMIGYDEKDVMTEKMKDKERIDYT |
| | | KDLSIDGLKGKKIGLLFSVDQQDENRKAVAEKIRKDLQDAGAILTDN |
| | | IQLSAEGVDNLQTLEYEFKHNVNDYLSQQKNVPVKSLEEIIAFNKKD |
| | | SKRRIKYGQTLIEGSEKSVITKEEFENVVQTSQENARKELDRYLVEKG |
| | | LDALVMINNDEVLLSAVAGYPELAVPAGYDKNGEPIGVVFVGKQFG |
| | | EKELFNIGYAYEQQSKNRKLPSL |
| indoleacetamide | gatA, iaaH | >AHCFPGON_04124 Glutamyl-tRNA(Gln) amidotransferase subunit A |
| hydrolase | | MSLFDHSVSELHKKLNSKEISVTDLVEESYKRIADVEDNVKAFLTLD |
| [EC:3.5.1.-] | | EENARAKAKELDAKIGAEDNGLLFGMPIGVKDNIVINGLRTTCASKI |
| | | LANFDPIYDATVVQKLKAADTITIGKLNMDEFAMGSSNENSGFYAT |
| | | KNPWNLDYVPGGSSGGSAAAVAAGEVLFSLGSDTGGSIRQPAAYCG |
| | | VVGLKPTYGRVSRYGLVAFASSLDQIGPITRTVEDNAYLLQAISGIDR |
| | | MDATSANVEVGNYLAGLTGDVKGLRIAVPKEYLGEGVGEEARESV |
| | | LAALKVLEGMGATWEEVSLPHSKYALATYYLLSSSEASANLSRFDG |
| | | VRYGVRSDNVNNLLDLYKNTRSEGFGDEVKRRIMLGTFALSSGYYD |
| | | AYYKKAQQVRTLIKNDFENVFANYDVIIGPTTPTPAFKVGEKVDDP |
| | | MTMYANDILTIPVNLAGVPAISVPCGFGANNMPLGLQIIGKHFDEATI |
| | | YRVAHAFEQATDYHTKKASL |
| indolepyruvate | ipdC | >AHCFPGON_00678 Indole-3-pyruvate decarboxylase |
| decarboxylase | | MKKQYTVSTYLLDRLHELGIEHIFGVPGDYNLAFLDDVVAHKNLKW |
| [EC:4.1.1.74] | | IGNCNELNAAYAADGYARIKGIAALITTFGVGELSAINGIAGSYAENV |
| | | PVIKITGTPPTKVMENGAIVHHTLGDGKFDHFSNMYREITIAQTNVTP |
| | | EHAAEEIDRVLRACWNEKRPGHINLPIDVYNKPINKPTEPIINKPILSN |
| | | KEALNKMLLHAISKINSAKKPVILADFEVDRFHAKESLHQFVEKTGF |
| | | PIATFSMGKGIFPEKHPQFIGVYTGDVSSPYLRKRIDESDCIISIGVKLT |
| | | DTITGGFTQGFTKEQVIEIHPYTVKIIDKKYGPVVMQDVLQHLSDSIE |
| | | HRNKDTLDVKPFILESSPFTEEFNPKAQMVTQKRFWQQMYHFLQEN |
| | | DVLIVEQGTPFFGSAAIPLPNNTAYVGQPLWGSIGYTLPALLGTQLAN |
| | | LSRRNILIIGDGSFQVTAQELSTILRQNLKPIIFLINNNGYTVERAIHGQ |
| | | NQLYNDIQMWDYNKLSMVFGSEEKSLTFKVENEAELAEVLTNITFN |
| | | KNQLIFIEVIMSQSDQPELLAKLGKRFGQQNS |
| arginine | speA | >AHCFPGON_01260 Arginine decarboxylase |
| decarboxylase | | MSQYETPLFTALVEHSKRNPIQFHIPGHKKGQGMDPTFREFIGHNAL |
| [EC:4.1.1.19] | | AIDLINIAPLDDLHHPKGMIKEAQDLAAAAFGADHTFFSIQGTSGAIM |
| | | TMVMSVCGPGDKILVPRNVHKSVMSAIIFSGAKPIFMHPEIDPKLGIS |
| | | HGITIQSVKKALEEHSDAKGLLVINPTYFGFAADLEQIVQLAHSYDIP |
| | | VLVDEAHGVHIHFHDELPMSAMQAGADMAATSVHKLGGSLTQSSIL |
| | | NVKEGLVNVKHVQSIISMLTTTSTSYILLASLDVARKRLATEGTALIE |
| | | QTIQLAEHVRDAINSIEHLYCPGKEMLGTDATFNYDPTKIIVSVKDLG |
| | | ITGHQAEVWLREQYNIEVELSDLYNILCLITLGDTESDTNTLIAALHD |
| | | LAATFRNRADKGVQIQVEIPEIPVLALSPRDAFYSETEVIPFENAAGRI |
| | | IADFVMVYPPGIPIFTPGEIITQENLEYIRKNLEAGLPVQGPEDMTLQT |
| | | LRVIKEYKPIS |
| arginine | speA | >AHCFPGON_05447 Arginine decarboxylase |
| decarboxylase | | MNQNRMPLYEALIEFKERGPLSFHVPGHKNGLNFPQEAIREFKDILSI |
| [EC:4.1.1.19] | | DVTELAGLDDLHSPFECIDEAQQLLAEVYNTKRSYFLINGSTVGNLA |
| | | MILSCCGEHDIVLVQRNCHKSIINALKLAGANPIFLDPWIDEAYNVPV |
| | | GVRNEIIKKAIEKYPNAKALILTHPNYYGMGMDLEASIAFAHAHKIP |
| | | VLVDEAHGAHLCLGEPFPKSALTYGADIVVHSAHKTLPAMTMGSYL |
| | | HINSHLVDEEKVTTYLSMLQSSSPSYPIMASLDIARFTMARIKEEGHS |
| | | EIVEFLRKFKEQLRSISQIAILEYPLQDELKVTVQTRCQLSGYELQSVF |
| | | EKVGIYTEMADPYNVLFILPLQVNGGYMKVIEMIRVALQHYEVKDK |
| | | RESIRYTYKGEFSLLPYTYKQLDGYETKIVSIEDAVGMIAAEMVIPYP |
| | | PGIPLIMYGERITSEHKEQIMYLERAGARFQCNTKYMKVYDIESRF |
| agmatinase | speB | >AHCFPGON_04579 Agmatinase |
| [EC:3.5.3.11] | | MRFDEAYSGKVFIKSHPSFEESKVVIYGMPMDWTVSYRPGSRFGPAR |
| | | IREVSIGLEEYSPYLDRELEEVKYFDAGDIPLPFGNAQRSLDMIEEYVS |
| | | KLLDADKFPLGLGGEHLVSWPIFKAMAKKYPDLAIIHMDAHTDLRE |
| | | SYEGEPLSHSTPIRKVCDLIGPENVYSFGIRSGMKEEFEWAKEVGMN |
| | | LYKFDVLEPLKEVLPKLAGRPVYVTIDIDVLDPAHAPGTGTLEAGGIT |
| | | SKELLDSIVAIANSNINVVGADLVEVAPVYDHSDQTPVAASKFVREM |
| | | LLGWVK |
| S- | speH, | >AHCFPGON_03112 S-adenosylmethionine decarboxylase proenzyme |
| adenosylmethi | speD, AMD1 | MDTMDTMGRHVIAELWDCDFDKLNDMPYIEQLFVDAALKAGAEV |
| onine | | REVAFHKFAPQGVSGVVIISESHLTIHSFPEHGY ASIDVYTCGDRIDPN |
| decarboxylase | | VAAEYIAEGLNAKTRESIELPRGTGSFEIKQRETKAL |
| [EC:4.1.1.50] | | |
| spermidine | speE, SRM | >AHCFPGON_04578 Polyamine aminopropyltransferase |
| synthase | | MELWFTEKQTKHFGITARINRTLHTEQTEFQKLDMVETEEFGNMLIL |
| [EC:2.5.1.16] | | DGMVMTTEKDEFVYHEMVAHVPLFTHPNPENVLVVGGGDGGVIRE |
| | | VLKHPSVKKATLVEIDGKVIEYSKQYLPSIAGALDNERVEVKVGDGF |
| | | LHIAESENEYDVIMVDSTEPVGPAVNLFTKGFYAGISKALKEDGIFVA |
| | | QTDNPWFTPELITTVFKDVKEIFPITRLYTANIPTYPSGLWTFTIGSKK |
| | | HDPLQVSEERFHEIETKYYTKELHNAAFALPKFVGDLIK |
| acetolactate | alsD, | >AHCFPGON_03760 Alpha-acetolactate decarboxylase |
| decarboxylase | budA, aldC | MTVAQLIDIDAKKTKTSNEVYQTSTMLALLDGIYDGVISFEDLKKHG |
| [EC:4.1.1.5] | | DFGIGTFDQLDGEMIAFDNGFYHLRSDGSAEKVEPEETTPFATVTFFE |
| | | KEMSYTVERSMNREEVEALLHELMPSKNLFYAIRMDGTFREVRTRT |
| | | VPRQEKPYTPLVEVTKSQPIFSFENTEGTLAGFWTPDYAQGIGVAGF |
| | | HLHYIDDERSGGGHVFDYVVENCTIQICQKAHMHLALPETADFMAA |
| | | ELSRENLEDNIATAEGAE |
| acetolactate | alsS | >AHCFPGON_03761 Acetolactate synthase |
| synthase, | | MSTGVKANDVKTKTKGADLVVDCLIKQGVTHVFGIPGAKIDSVFDV |
| catabolic | | LQERGPELIVCRHEQNAAFMAAAIGRLTGKPGVCLVTSGPGTSNLAT |
| | | GLVTANAESDPVVALAGAVPRTDRLKRTHQSMDNAALFEPITKYSV |
| | | EVEHPDNVPEALSNAFRSATSTNPGATLVSLPQDVMTAETTVESIGA |
| | | LSKPQLGIAPTHDITYVVEKIKSAKLPVILLGMRASTNEVTKAVRKLI |
| | | ADTELPVVETYQAAGAISRELEDHFFGRVGLFRNQPGDILLEEADLVI |
| | | SIGYDPIEYDPKFWNKLGDRTIIHLDDHQADIDHDYQPERELIGDIAL |
| | | TVNSIAEKLPKLVLSTKSEAVLERLRAKLSEQAEVPNRASEGVTHPL |
| | | QVIRTLRSLISDDTTVTCDIGSHSIWMARCFRSYEPRRLLFSNGMQTL |
| | | GVALPWAIAATLVEPGKKVVSVSGDGGFLFSAMELETAVRLNSPIVH |
| | | LVWRDGTYDMVAFQQMMKYGRTSATEFGDVDLVKYAESFGALGL |
| | | RVNTPDELEGVLKEALAADGPVIIDIPIDYRDNIKLSEKLLPNQLN |
| acetolactate | ilvH, ilvN | >AHCFPGON_02519 Acetolactate synthase small subunit |
| synthase | | MKRIVTATVRNQSGVLNRITGVMTRRHFNIESISVGHTESSDISRMTI |
| [EC:2.2.1.6] | | VVHVESEQQVEQLIKQLHKQIDVLKVSDITEEAMIARELALIKVATSV |
| | | ARAELYSLIEPFRAAVIDVGKDSIVVQVTGTQDKVEALIELLRPYGLK |
| | | EIARTGVTAFTRSMKKQDKQVMLIQ |
| | | >AHCFPGON_02520 Acetolactate synthase large subunit |
| | | MSSKTEEKLATGAQLLLEALEKEGVEVIFGYPGGAVLPLYDALYDC |
| | | EIPHILTRHEQGAIHAAEGYARITGHPGVVIATSGPGATNVITGLADA |
| | | MIDSLPLVVFTGQVATTLIGSDAFQEADIMGLTMPVTKHNYQVRKA |
| | | SDLPRIIKEAFHIAKTGRPGPVVIDLPKDMVVEKGEQCSNVQMDLPG |
| | | YNPNYEPNLLQINKLLKVIETAKKPLILAGAGILHAKASKELTNFARK |
| | | YEIPVVHTLLGLGGFPPDDELFLGMGGMHGSYTANMALYECDLLINI |
| | | GARFDDRLTGNLAYFAKGATVAHIDIDPAEIGKNVPTEIPIVASAKRA |
| | | LEVLLEPEGGKENHHEWITLLKGRKEQYPFSYKRNSESIKPQYAIDM |
| | | LYEITKGEAIVTTDVGQHQMWAAQYYPLKNPDKWVTSGGLGTMGF |
| | | GFPAAIGAQIAKPEELVIAIVGDAGFQMTLQELSVLKEHALPVKVFIL |
| | | NNEALGMVRQWQDEFYNQRYSHSLLPCQPDFVALANAYGIKGIRID |
| | | DPLLAKKQIQHAIELQEPVVIDCRVLQSEKVMPMVAPGKGVHQMEG |
| | | VEKG |
| acetolactate | ilvH, ilvN | >AHCFPGON_04053 Acetolactate synthase small subunit |
| synthase | | MSHTFSLVIHNEPSVLLRISGIFARRGYYISSLHLNERDTSGVSEMKLT |
| [EC:2.2.1.6] | | AVCTENEATLLVSQLKKLIDVLQVNKL |
| | ilvB, | >AHCFPGON_04054 Acetolactate synthase large subunit |
| | ilvG, ilvI | MKQQYTAYEKLQCEEMTGAGHVIQGLKKLGVTTVFGYPGGAILPV |
| | | YDALYESGLKHVLTRHEQAAIHAAEGYARASGKVGVAFATSGPGAT |
| | | NLVTGLADAYMDSIPLVVITGQVATPLIGKDGFQEADVVGITVPVTK |
| | | HNYQVRDVNHVSRIVQEAFYIAKSGRPGPVLIDIPKDVQNAKVTSFF |
| | | NEEVDIPGYKPELVPDSMKLREVAKAISKSKRPLLYIGGGVIHSGGSD |
| | | ELFEFARENRIPVVSTLMGLGAYPPGDPLFLGMLGMHGTYAANMAV |
| | | TECDLLLALGVRFDDRVTGKLELFSPHSKKVHIDIDPSEFHKNVTVE |
| | | HPIVGDVKKALHMLLHMSIYTQTDEWLQKVKAWKEEYPLSYKQKE |
| | | SELKPQHVINLVSELTNGEAIVTTEVGQHQMWAAHFYKARKPRTFL |
| | | TSGGLGTMGFGFPAAIGAQLAKEEELVVCIAGDASFQMNIQELQTIA |
| | | ENNIPVKVFIINNRFLGMVRQWQEMFYENRLSESKIGSPDFVKVAEA |
| | | YGVKGLRATNSTEAKQVVLEAFAHEGPVVVDFCVEEGENVFPMVLP |
| | | NKGNNEMIMKRWEE |
| hydrogen | hcnA | >AHCFPGON_00274 hypothetical protein |
| cyanide | | MSRITHHPILGTLQSSKRITFQFNGHQYKAYEHETIAAALLANGIRTV |
| synthase | | RVHEDSGTPQGIYCNIGHCSECRMTVNNQTNVRACLTVVEENMVVE |
| [EC:1.4.99.5] | | SGKQHPNIVREMVKKR |
| | hcnB | >AHCFPGON_00273 Hydrogen cyanide synthase subunit HcnB |
| | | MSDVIIIGAGPAGLSASISCARFGLKVLVIDEFMKPGGRLLGQLHQEP |
| | | TGEWWNGIKESKRLHEEAESLSVHIRCGVSVYNLDRDENNWFVHTN |
| | | IGTLEAPFVLLATGAAEYSIPLPGWTLPGVMSIGAAQVMTNVHRVQV |
| | | GKKGIIIGANILSFAILSELQLAGITVDHIVLPEKSELSQKAGEPEEVLN |
| | | SLLNAAHLAPSAILRIGSHFMKYDWIRKAGLTFYPNSGMKINGTPLH |
| | | LRKAALEIIGTDQVEGVRVANIDSKGNVINGSEKIYEADFVCIAGGLY |
| | | PLAELAAVAGCPFHYISELGGHVPLHSETMETPLPGLFVAGNITGIES |
| | | GKIAMAQGTVAGLSIAKYASKKRDIVDQQLYHAIQNVHSVRQKAAI |
| | | QFNPMVDIGRRKMNDLWHKFQTNDKDFYKQQEII |
| | hcnC | >AHCFPGON_00277 Hydrogen cyanide synthase subunit HenC |
| | | MRHCDVLIIGGGIIGCSIAYYTSKYGRDVTIIEKGEFVSGTSSRCDGNI |
| | | LAIDKDPGFDSQMSLVSQKLVTDLSEELEHSFEYRAPGSILVCESDEE |
| | | MEAAQQWVNRQQEAGLPFRMLDRQDIREESPFFADDLLGGLECATD |
| | | STVNPYLLAFSLLSEAQKFGAKAFKQTEVKSMEIETDGSFVVETTNG |
| | | TFTAQQVVNAAGVWAPKIGQMLNINIPIEPRKGHIIVASRQQHVGCR |
| | | KVMEFGYLISKFGGKRKVDALTEKYGVALVFEPTESQNFLIGSSREF |
| | | VGFHTRINNEVIKCIANRAIRFYPKMADMMVIRSYAGLRPWTEDHLP |
| | | IISRVEHIPNYFIAAGHEGDGISLAAVTGKVIEELLNEKETIIPIEPLRLS |
| | | RFTERVLNK |
| flagellar | flgC | >AHCFPGON_04666 Flagellar basal-body rod protein FlgC |
| basal-body | | MFQAINASGSGLTTARKWMEVTSNNIVNANTTAAPGADLYERRSVV |
| rod protein | | LESNNSFASMLDGAPTNGVKIKSIEADKTENLVYDPTHPHANEEGYV |
| FlgC | | RYPNIDVTAEMTNVMVAQKMYEANTSVLNANKKMLDKDLEIGRG |
| flagellar | flgD | >AHCFPGON_04674 hypothetical protein |
| basal-body | | MPTVGLNTTSTNHIPLQAGAQTKNASVNGVQSPVQQTNGVSASNQK |
| rod | | TPGIMDKDDFLKLFLASFQHQDPFNAMDMNQMMNQTAQLSLMEQV |
| modification | | QNMTKAVDKLQSTMYTTALDGGMKFLGKYVRGINNKGEQVTGQV |
| protein FlgD | | ETVRLAENNDVQLIVDNQVVSLRFVERVSDKPIAETNPEDEKKDDIE |
| | | KNEEVKQN |
| flagellar hook | flgE | >AHCFPGON_04675 Flagellar hook protein FlgE |
| protein FlgE | | MIKALYTSITGMNAAQNALSVTSNNIANAQTVGYKKQKAIFDDLLY |
| | | NNTVGSRGDGAYAGTNPKSIGNGVKFSGTSTDFSDGSITLTSDKMET |
| | | AIEGNGLFLVGDRNSGNVEYTRKGSFGVSKDNYVTNTSGQYVLGYG |
| | | VKTGTQEIDFSSRPSPIHIPMGSAVGGIQTDKATIGGNLPRNQNALSH |
| | | EFTVFDEEGNSLTLRVNIKQKTTKETVDGKEVEKPVPGEYTYTVSVR |
| | | NDSKNEKEFKPVEGMTGEKNLKFDTLGNLKETDEAVQKNPVTGEIT |
| | | KGGTVKIPFGKGLTLDLSGLTNYPTGKTISTTEVTGRPAAIANDYSIS |
| | | DGGFVMMRYSDGSMKVVGQLAVATFPNSGGLMKTGNGNYIATPSA |
| | | GIPGIGVAGENGAGNVRGSAKESSNVDLSVEFVDLMLYQRGFQGNA |
| | | KVIKVSDEVLNEVVNLIR |
| flagellar hook- | flgK | >AHCPGON_04660 hypothetical protein |
| associated | | MRLSDYNTPLSGMLAAQMGLQTTKQNLSNIHTPGYVRQMVNYGSA |
| protein 1 FlgK | | GGSKGYAPEQRIGYGVQTLGVDRITDEVKTKQYNDQMSQFSYYAY |
| | | MNSTLSRVESMVGTTGKNSLSSLMDGFFNAFREVAKNPEQSNYYDT |
| | | LIAETGKFTSQVSRLAKNLDTVEAQTTEDIEAHVNEFNRLAASLAEA |
| | | NKKIGQAGTQVPNQLLDERDRIMTEMSKYADIEVSYEATNPNIASVR |
| | | MNGVLTVNGQDTYPLQLQKDKKPMSVQISGTDIPLSGGTILSAIDTK |
| | | AKITNYKDNLNEFVNSLKKQVNNTMKKELFVGEDAKKLELNPDFIK |
| | | DISKMKISAETANNLAAITDKGYKDGLTYKQALDQFLVGVASDKSS |
| | | VNAYQNIHKDLLEGIQQEKMSIEGVNMEEEMVNLMAFQKYFVANS |
| | | KAITTMNEVFDSLFSIIR |
| flagellar hook- | flgL | >AHCFPGON_04661 Flagellar hook-associated protein 3 |
| associated | | MRVSTFQNANWAKNQLMDLNVQQQYHRNQVTSGKKNLLMSEDPL |
| protein 3 FlgL | | AASKSFAIQHSLANMEQMQKDIADSKNVLTQTENTLQGVLKSLTRA |
| | | DQLTVQALNGTNSEKELQAIGVEIDQILKQVVYLANTKEQGRYIFGG |
| | | DSAKNPPFTEDGTYQGGKNDVNWKLNDGYEFKAFRNGGALLSPVIK |
| | | TLKQMSEAMKNGDQKALKPLLEGNKQNLDGIINRTTEVGSTMNTM |
| | | ETFKTILNEQNVALQENRKEIEDVDLAVAISDLAYINATYEATLKAVS |
| | | TMSKTSILDYM |
| flagellar | flhA | >AHCFPGON_04694 Flagellar biosynthesis protein FlhA |
| biosynthesis | | MFKIESARTYFSIFLAASFVVALLIPLPPFILDIIIVFLLSMSVLIYMRAT |
| protein FlhA | | SINEWDELKSFPTMLLLIGIFRVSINVSTTRAILTDGNAGHVIEEFGQF |
| | | VIGGNLLIGIVIFTVLIIFQFIVANGASRTAEVAARFTLDSLPGKQMSID |
| | | ADLNQRIISEKDAQAKRKKLNMETEFYGAMDGAGKFIKGDVIFGIVI |
| | | LFVNIIFGLIVGMMQQGMSFADAALHYTQLTVGDGIVNQIGSLMLAI |
| | | STGIIVTRVFDGSPDTVTEGIFKELLAHEVVVYALGGLFIAMGIFTPLP |
| | | FLPFALVGGTIIFLGIRNKNRIKKEKEDELQKEIEMIQGEDEQLQQVED |
| | | SFGVFTDKYPIIVELGLDLAALVKQKINGETARDKVVLMRKSIITDLG |
| | | INVPGINFKDNTSFRPRGRYIIRIKGAKAAEGVLKSGYLLALKTPNVM |
| | | ADLDAEPAKDPIFGEDGYWILEHMVQDAQMKGYQVLEPLSILITHLD |
| | | VVVRRNLHELIQRQHVKDLINSLENDNGVLLEEIKKKEIDLSLVQNVI |
| | | KQLLKEGISIRDLPTIIEGIIDGKEIYQNHVDGVTSFVRECISKVICENA |
| | | KNPDGKIYAALFSDSIELDADVVNNSYQGYLLNWDLDLETRVVEQV |
| | | QRVFKQARLMGREPVLLTRRKDFRFAIVRLLERYQVEAQVLCISELA |
| | | PEIVVDQIAYIE |
| flagellar | flhB | >AHCFPGON_04693 Flagellar biosynthetic protein FlhB |
| biosynthetic | | MAKDNKTEKATPQKRKKSREEGNIARSKDLNNLFSILVLAVVVYFF |
| protein FlhB | | GDWLGFEIANSVSVLFNQIGKNTDSTEYFYLMGILLLKVSAPILILVY |
| | | AFHLFNYMIQVGFLFSSKVIKPKASRINPKNYFTRLFSRKSLVDILKSL |
| | | FYMGLIGYVAYVLFKKNLEKIVSMIGFNWTASLTEIIRQIKFIFLAILII |
| | | LIVLSIIDFIYQKWEYEQDIKMKKEEVKQEHKDNEGDPQVKGKRKNF |
| | | MHAILQGTIAKKMDGATFIVNNPTHISVVLRYNKHVDAAPIVVAKG |
| | | EDELALYIRTLAREQEIPMVENRPLARSLYYQVEEDETIPEDLYVAVI |
| | | EVMRYLIQTNELEV |
| flagellin | hag, fliC | >AHCFPGON_04680 Flagellin |
| | | MRIGTNVLNMNARQSLYENEKRMNVAMEHLATGKKLNHASDNPA |
| | | NIAIVTRMHARASGMRVAIRNNKDAISMLRTAEAALQTVTNILQHM |
| | | RDLAVQSSNGTNSNKNRNSLHKEFQSLTEEIGYIVETTEFNDLSVFDG |
| | | QNRPITLDDNGHTINMMKHIPPSPTQHDIKISTEQEARAAILKIEEALQ |
| | | SVSLHRADLGAMINRLQFNIENLNNQSMALTDAASRIEDAEMAQEM |
| | | SDFLKFKLLTEVAICMVSQANQIPQMVSKLLQS |
| flagellin | hag, fliC | >AHCFPGON_04681 Flagellin |
| | | MRINTNINSMRTQEYMRQNQDKMNTSMNRLSSGKSINSAADDAAG |
| | | LAIATRMRAKEGGLNVGARNTQDAMSALRTTDSALNSISNILLRMR |
| | | DLATQSANGTNDGKDKDSLNLEFKELQEEINHIAGKTNFNGKNLLA |
| | | AAGTDKNISIQLSDVASDNLTITAVDATTGATGLGLTKTIGAPAAPVI |
| | | PAPPAPAADNDAAGAIVQLDAAIQKVADMRATFGSQLNRLDHNLN |
| | | NVNSQATNMAASASQIEDADMAKEMSEMTKFKILNEAGISMLSQAN |
| | | QTPQMVSKLLQ |
| flagellin | hag, fliC | >AHCFPGON_04682 Flagellin |
| | | MRINTNINSMRTQEYMRQNQDKMNTSMNRLSSGKSINSAADDAAG |
| | | LAIATRMRAKEGGLNVGARNTQDAMSALRTTDSALNSISNILLRMR |
| | | DLATQSANGTNDGKDQDSLNLEFKELQGEIDHIAGKTNFNGKSLLA |
| | | AKGTDIDIQLSDISGDKLTIASVDATTGADGLNLTKTIASTGKGGDAA |
| | | ESIKELDKAIQSVADMRATFGSQLNRLDHNLNNVNSQATNMAASAS |
| | | QIEDADMAKEMSNMTKFKILNEAGISMLSQANQTPQMVSKLLQ |
| flagellin | hag, fliC | >AHCFPGON_04683 Flagellin |
| | | MRINTNINSMRTQEYMRQNQDKMNTSMNRLSSGKSINSAADDAAG |
| | | LAIATRMRAKEGGLNVGARNTQDAMSALRTTDSALNSISNILLRMR |
| | | DIATQSANGTNETKDQASLDLEFQELHKEIDHIAEKTNFNGKNLLAA |
| | | TGADIDIQLSDVSGDKLTIASINAKADTLLTATGTNVKTTADASTAIT |
| | | NLDKAIQTVADNRATFGSQLNRLDHNLNNVNSQSTNMAAAASQIED |
| | | ADMAKEMSNMTKFKILNEAGISMLSQANQTPQMVSKLLQ |
| flagellin | hag, fliC | >AHCFPGON_04684 Flagellin |
| | | MRINTNINSMRTQEYMRQNQDKMNTAMNRLSSGKSINSAADDAAG |
| | | LAIATRMRAKEGGLNVGARNTQDAMSALRTTDSALNSISNILLRMR |
| | | DIATQSANGTNDTKDQDSLDLEFQELKGEITHIAEKTNFNGKTLLAG |
| | | VAAANNIDVQLSDVSGDKLTITAIDATAATLKITGDVKSTKNASDSIT |
| | | ALDAAIQTVADNRATFGSQLNRLDHNLNNVNSQSTNMAAAASQIED |
| | | ADMAKEMSNMTKFKILNEAGISMLSQANQTPQMVSKLLQ |
| flagellar hook- | fliD | >AHCFPGON_04662 B-type flagellar hook-associated protein 2 |
| associated | | MAGTISNYGDRQQIWNLGNNIIDTKKLVDLELQALEMKKSPYTTQK |
| protein 2 | | QTLTNENKVYASMKKEFANFVQVFKDLNTFKGDEKKTTLSKDGFM |
| | | TAQADAAAIPGTYTITVERVAERHQITTAPLTPPKTPEGTEQKFSLDL |
| | | KLGVDDVFQINGKEVKISKDMTYKDLVNKINNGNYGASVYTLGDQ |
| | | LFFTSTTAGEAGELKLTDGANGFLQNIGLVTSAKNPDGTNVVAHQV |
| | | TGAINAEYTINGIKGTSKTNKIDTIPGLTINLEKVTTEPIKLTIEDSDIKN |
| | | SIDLIKKMKDEYNNAVKSLDLFSGENGVMQGNNVSFAISNAMTSIFK |
| | | FSQDDKYLFSFGIQIDKTGNMTLDEEKLKIAFKENPESTKQFFFGENGI |
| | | GHDIDKKLEGIFGDEGIIGKRSKSIEKQVTDLERKIQDIDTINKKKQESI |
| | | IDKYAKLESQLALLDSQLQTIKAMTKTKSDD |
| flagellar hook- | fliD | >AHCFPGON_05877 Flagellar hook-associated protein 2 |
| associated | | MAQISQRAGKATDTSLDNYTLGKRMKDIDSRITNFERRLELTEARY |
| protein 2 | | WRQFSEMERAISMMNQQSSMLMSNFGSGMAQG |
| flagellar hook- | fliE | >AHCFPGON_04667 Flagellar hook-basal body complex protein FliE |
| basal body | | MKIQPMLHTQPFGAIQSIGAPKTSQTSVVEGKKFIDLLEDMNQTQNN |
| complex | | AQTAVYDLLTKGVGETHDVLIQQKKAESQMKTAALVRDNLIENYKS |
| protein FliE | | LINMQI |
| flagellar M- | fliF | >AHCFPGON_04668 hypothetical protein |
| ring protein | | MEKMKNVIQSLKTWHKLVIGAALLAIVTGALLYFTLPDKYVVVYQN |
| FliF | | LNDADKQEITAELSKLGVDYQLAADGSIRVQKNDAPWVRKEMNGM |
| | | GLPFNSKSGEEILLESSLGSSEQDKKMKQIVGTKKQLEQDIVRNFATV |
| | | ETANVQITLPEKETIFDEEKAKGTAAITVGVKRGQLLTADQVAGIQQ |
| | | MISAAVPGVKAEEVSVIDSKKGVISKGADEAHSSSSSSYEKEVEMQH |
| | | QLEGKLKQDIDATLMTMFKPNEYKVNTKVSVNYDEVTRQSEKYGD |
| | | KGVLRSKQEQEESSTAQEGADTKQGAGITANGEVPNYGTNNNQNG |
| | | KVVYDNKNGNKIENYEIDKTVETIKKHPELTKTNVVVWVDNDTLVK |
| | | RKIDMTTFKEAIGTAAGLQADPNGNFINGQVNVVTVQFDQPKAEKE |
| | | KEPEKSGMNWWLFGGITAGLLAIGGLVWFLLARRKRKKEEEEYEEY |
| | | LAEEEIAASNESILEIPEEKIVPEPKPEPEEPKEPTLDEQVQDATKEHVE |
| | | GTAKVIKKWLNGQ |
| flagellar | fliG | >AHCFPGON_04669 Flagellar motor switch protein FliG |
| motor switch | | MLDEISSKEKAAILIRTLEEGVAAKVIEYMTAEEKEVLLREIAKFRVY |
| protein FliG | | KPETLENVLGEFLYELNVKELNLVTPDKEYIRRIFKNMPEDELEKLLE |
| | | DLWYNKDNPFEFLNSLTDLEPLLTVLNDESPQTIAIIASYIKPQLASQL |
| | | IERLPDHKRVETVMGIAKLEQVDGELINQIGELLKSKLNNMAFSAIN |
| | | KTDGLKTIVNILNNVSRGVEKTVFQKLDEVDYELSEKIKENMFVFED |
| | | LLGLEDLALRRVLEEITDNGVLAKALKIAKEEIKEKLFTCMSSNRKE |
| | | MILEELDGLGPLKMTDAEKAQQTITGTVKKLEKEGRIIVQRGEEDVLI |
| flagellum- | fliI | >AHCFPGON_04671 putative ATP synthase YscN |
| specific ATP | | MSRLLMNENEKWNKFIETPLYTKVGKVHSVQEQFFVAKGPKAKIGD |
| synthase | | VCFVGEHNVLCEVIAIEKENNMLLPFEQTEKVCYGDSVTLVSEDVVV |
| [EC:7.4.2.8] | | PRGNHLLGKVLSANGEVLNEEAENIPLQKIKLDAPPIHAFEREEITDV |
| | | FGTGIKSIDSMLTIGIGQKIGIFAGSGVGKSTLLGMIAKNAKADINVIS |
| | | LVGERGREVKDFIRKELGEEGMRKSVVVVATSDESHLMQLRAAKLA |
| | | TSIAEYFRDQGNNVLLMMDSVTRFADARRSVDIAVKELPIGGKTLL |
| | | MESYMKKLLERSGKTQKGSITGIYTVLVDGDDLNGPVPDLARGILD |
| | | GHIVLKRELATLSHYPAISVLDSVSRIMEEIVSPHHWQLANDVRKILSI |
| | | YKENELYFKLGTIQQNEENAYIFECKNKVEGINTFLKQGRSDSFQFD |
| | | DIVEAIQHIV |
| flagellar | fliM | >AHCFPGON_04687 Flagellar motor switch protein FliM |
| motor switch | | MSGEKLSQEQIDALLKAVNEGEEMPAFAQEAGKQEKFQEYDFNRPE |
| protein FliM | | KFGVEHLRSLQAIASTFGKQTSQTLSARMRIPIELEPSTVEQVPFTSEY |
| | | VEKMPKDYYLYCVIDLGLPELGEIVIEIDLAFVIYIHECWLGGDSKRN |
| | | FTMRRPLTAFEFLTLDNIFLLLCKNLEQSFESVVAIEPKFVTTETDPNA |
| | | LKITTASDIISLLNVNMKTDFWNTTVRIGIPFLSVEEIMDKLTSENIVE |
| | | HSSDKRKKYTSEVEVKVNQVYKPVHVAIGEQKMTMSEIEQIEEGDII |
| | | PLHTKVSDELLGYVDGKHKFNCFIGKDGTRKALLFKSFVE |
| flagellar | flip | >AHCFPGON_04690 hypothetical protein |
| biosynthetic | | MRIKKQLSLLAVIFVFSIVFSIIFVNPAYAAPNGFINFENGKEFTSNSSV |
| protein FliP | | QLFALVTLLSLSSSIVLLFTHFTYFMIVLGITRQGLGVMNLPPNQVLV |
| | | GLALFLSLFTMQPVLGQLKSDVWDPMTKEKITVSQAAETTAPIMKE |
| | | YMSKHTYKHDLKMMLKVRGEELPKDLKDLSLFTLVPSFTLTQIQKG |
| | | LLTGMFIYLAFVFIDLIISTLLMYLGMMMVPPMILSLPFKILVFVYLG |
| | | GYTKIVDIMFKTVA |
| flagellar | fliQ | >AHCFPGON_04691 hypothetical protein |
| biosynthetic | | MNTSPIIDIFQTFFYKGVMILMPVAGVSMIVVIIIAVIMAMMQIQEQTL |
| protein FliQ | | TFLPKMASIVLVIIILGPWMFQELTTLILDLFDKIPSLLRSY |
| flagellar | fliR | >AHCFPGON_04692 Flagellar biosynthetic protein FliR |
| biosynthetic | | MNMELWAATFFAFCRITSFLYFLPFFSGRSIPAMAKVTVGLALSITVA |
| protein FliR | | DQVDVSHIKTVWDVAAYAGTQIVIGLSLSKIVEMLWNIPKMAGHIL |
| | | DFDIGLSQASLFDVNAGSQSTLLSTIFDIFFLIIFISLGGINYFVATILKSF |
| | | QYTEAISKLLTTSFLDSLLATLLFAITSAVEIALPLMGSLFIINFVLILIA |
| | | KNAPQLNVFMNAYVIKITCGILFIAMSVPMLGYVFKNMTDVLLEEYT |
| | | KLFNFFLTK |
| flagellar | fliS | >AHCFPGON_04663 hypothetical protein |
| protein FliS | | MQAWQRYMQNDIMTSNPIKNTIFIYERCIVEFRKLEELLNTFKLQEG |
| | | DDLLEKLERIFEELKLQLNPDISKDLYDSLFGLYDWISIQIQTMKVTR |
| | | EAKDIDAIVQVLQDLIDGYRGALENEQ |
| Chemotaxis | motA | >AHCFPGON_03184 Chemotaxis protein PomA |
| protein | | MDFATIIGLILGFVAVVVGMVVKGADITALLNPAAALIIFIGTFAAVCI |
| | | AFPMNQLKRVPKLFKVLFGSNKKDLSYEQLLELFVHWTSESRKYGIL |
| | | SLEQQLDKIQDEFLLRGMKFVIDGVSAEDLEQILESELEAIEERHAKG |
| | | AAIFSQAGTYAPTLGVLGAVIGLVAALGNLTDIEKLGHAISGAFIATIF |
| | | GIFSGYVLWHPFANKLKQKSSAEIEKKRLIIDCLLMLQEGTYPFIMKN |
| | | RILGALSATERKKLEKGAEKNAE |
| | motB | >AHCFPGON_03185 Motility protein B |
| | | MRSKKNRRGKKKKHDEHIDETWLIPYSDMLTLLFALFIVLFAMSSID |
| | | AAKFKQMAVAFRSELAGGTGNKEFLSDQKPNDEKELSASSLEAEQT |
| | | KKQEEARAKEKKEMDELKALQKKIDQYINEKQLSSSFQTKLTEKGL |
| | | MVTILENILFDSGKADVKLESLGIAKEMSSLLVSASPREITVSGHTDN |
| | | VPIANAQFASNWELSTQRAVNFMQVLLQNKELQPEKFSAIGYGEYR |
| | | SIAPNDTQEGKAKNRRVEVFILPLTEKVK |
| Chemotaxis | motA | >AHCFPGON_04649 Chemotaxis protein PomA |
| protein | | MGEKNQVLARPQRRKRKFDISSPVGIIVGFIIVIAAIMLGGGGIKAFKN |
| | | FLDISSILIVIGGTTATIVVAYRFGEIKKYTKSIFTVLHRREEDLEQLTD |
| | | LFVDFSKKSKKNGLLSLEVDGEQVDNPFIQKGIRLMLSGYDEDELKE |
| | | VLLKDIETEVYELRKGAALLDKIGDFAPAWGMIGTLIGLIIMLQNLQ |
| | | DTSQIGTGMAVAMLTTLYGSVLANMIAIPLAEKVYRGIEDLYTEKKF |
| | | VIEAISELYRGQIPSKLKLKLDTYVYETKVKKVKGAA |
| | motB | >AHCFPGON_04650 Motility protein B |
| | | MSKGPQKGSPRWMTTFTDLTMLLLTFFVLLVATSKQDAVKLSKMLE |
| | | KFSDTGQVDAKVMENTIPDISHEKNDEKMISKKRMDELYKKLKAYV |
| | | DNNGISQVNVYREDTGVSVVIVDNLIFDTGDANVKPEAKGIISQLVG |
| | | FFQSVPNPIVVEGHTDSRPIHNEKFPSNWELSSARAANMIHHLIEVYN |
| | | VDDKRLAAVGYADTKPIVPNDSPQNWEKNRRVVIYIKE |
| two- | cheA | >AHCFPGON_04652 Chemotaxis protein CheA |
| component | | MQTDLLNIFFEESEEHLQSLNENVLVLEQNPADMDVVGEIFRSAHTF |
| system, | | KGMSASMEFTEMADLTHKMENVLDEIRHGNIVVNADIIDVIFECIDN |
| chemotaxis | | LEKMVADVQQGGMGNIDVASTKQKLEALLNGNVETPTEHIEQNHID |
| family, sensor | | TDDAVSHEVHITVEQQAILKAVRAIMCIEALQNVGNIQKTAPSIEEIE |
| kinase CheA | | ADAFGFEFTVFMDTDCSIEELKQVVLHVSEIEKVEVKQGEPISKEVAS |
| [EC:2.7.13.3] | | KKVVTQEVVQVEEKLQPAVVTQVNSPIEATNQPSSTMPAKSTTKTK |
| | | NAKVENRSIRVQLEKIERLMNMFEESVIERGRIDELAQTIQNKELIEH |
| | | LNRLGDISKDIQNVLLNMRMVPIETVFNRFPRMVRMLAKDLGKKID |
| | | LQITGEDTEVDKIVIDEIGDPLVHLIRNAIDHGVETVEKRRDAGKNET |
| | | GTIKLEAFHSGNHVVIQITDDGNGINKGKVLEKAIKNGVVTEADANR |
| | | LTDREVFDLIFQPGFSTAEVVSDLSGRGVGLDVVKHTIHSLGGHLIID |
| | | SEEGKGSTFRIELPLTLSIIQSMLVQTNDKRYALPLGNIVEAIRIKREDI |
| | | QSLQGKDVLNYRNQIIEVKHLSTVFGEKTVDEAFASYDGQMVPVLIV |
| | | RNTHRSYGLIVNTIIGQREIVLKSLGDFFAESSNYFSGATILGDGRVVL |
| | | ILNPEGL |
| chemotaxis | cheR | >AHCFPGON_03633 Chemotaxis protein methyltransferase |
| protein | | MENKYYNFDPSVDTDERTNLEIELLLEAVFKLSGFDFRQYARTSIYR |
| methyltransfer | | RICNRMQLSNIPTISKLIEKVIHEEGVLEQLLNDFSINVTEMFRNPAFF |
| ase CheR | | KALREHVIPELKKQPEIRIWHAGCATGEEVLSMSILLHEEGLSEKSVI |
| [EC:2.1.1.80] | | YATDMNTNVLEKAKQAILPLNKMQTYTKNYLQAGGTQAFSNYYST |
| | | DNRFAYFNPSLLQNIIFAQHNLVTDQSFNEFHIILCRNVLIYFTSKLQN |
| | | QVQHLFYESLSHNGFLCLGNKETLRFSNIMPHYTQFNPSEQIYQKIQ |
| chemotaxis | cheR | >AHCFPGON_04656 Chemotaxis protein methyltransferase |
| protein | | MPIVNMIIEQDYDHFIASFKQQFNMDIASYKQDRMRRRIDAFISRKGF |
| methyltransfer | | ENYTNFLNKLRADQNLFLNFIDYITINVSEFFRNKERWQTLESKALPK |
| ase CheR | | LLEQNSGKLKVWSAACAAGEEPYTLSLILSKHLAPFRFEIQATDLDF |
| [EC:2.1.1.80] | | HILETAKRAQYTERSLKELPTDLKERHFTKENGLYSLHQNIKQNVSF |
| | | KQHDLLMQSFDTNYDLIICRNVMIYFTEEARIKLYEKFSRSLRKGGV |
| | | LFVGSTEQILTPERYNLQRFDTFFYEKI |
| two- | cheY | >AHCFPGON_04651 Chemotaxis protein CheY |
| component | | MAHKILVVDDAMFMRTMIKNLLKSNAEFEVIGEAENGVEAIQKYKE |
| system, | | LQPDIVTLDITMPEMDGLEALKEIIKIDSSAKVVICSAMGQQGMVLD |
| chemotaxis | | AIKGGAKDFIVKPFQADRVIEALTKVANS |
| family, | | |
| chemotaxis | | |
| protein CheY | | |
| purine-binding | cheW | >AHCFPGON_04678 Chemotaxis protein CheV |
| chemotaxis | | MSQAQSILLESGTNELEIVTYTVGENLFSINVMKVREIINPFPVTTVPE |
| protein CheW | | SHHAVEGVVQVRGEILPVINLAMALNLKSTKPLDQTKFIISELNQMK |
| | | VIFRVDEVHRIQRISWEQIDEPASLSMGLEETTSGIVKLDGKIILLLDY |
| | | EKIVCEISGTGYDNKSIAGLEQKTDRAEKVIYIAEDSAMLRQILEETLS |
| | | SAGYTKMNFFSNGAEALAQIEKLAKEQGEKMFEHIHLLITDIEMPKM |
| | | DGHHLTKVVKDSEVMNRLPVIIFSSLITNELFHKGEAVGANAQVSKP |
| | | DIQELIGLVDKLVL |
| Flagellin | hag, fliC | >LOFDPPFF_01689 Flagellin C |
| | | ATGATTATCAATCACAACATTACAGCACTTAACACACACAACAAA |
| | | CTTTCGAGCGCATCTTCTGCTCAAAGTAAATCGATGGAGAAATTA |
| | | GCTTCAGGTCTTCGCATCAATAAAGCAGGCGACGACGCTGCTGGT |
| | | CTTGCGATTTCTGAAAAAATGCGTGCACAGGTTCGTGGACTTGAC |
| | | CAAGCTTCACGTAACGCACAAGACGGAATCTCAATGATTCAAAC |
| | | AGCTGAAGGTGCCTTAAACGAAACACACGATATTCTTCAACGTAT |
| | | GCGTGAATTAGCAGTTCAGGGTGCAAACGATACGAACGTTACTCA |
| | | AGACCGCGACGCTATTCAAGAAGAATTAACAGCATTAAAGAGCG |
| | | AAATCGACCGTATCGGTGAAACAACAGAATTCAACAAACAGACA |
| | | CTTTTAAATGGTGGACTTGGTGGAACTGTTGATCAAGATGTTGCA |
| | | ACTACTACAGTTTTAGGTGTTACAGGTGTAGCAGGTGCTTCTACT |
| | | AACGGTGCTGTCGCTGGATCATATGCAATCACTAGCGGAACTGCT |
| | | GGCGAATTGACAATGACATTCGGAACTAAAACGCAAACAATCAG |
| | | CAACGCAAACGGCGCTCAAGACTTGAACTTCTCTGAGTTCGGTAT |
| | | CTCGATCAAAACAAACGCTGGTTACACTGCAGATGACGCAGTTGG |
| | | TAACGTTGTTGTAGATCCTGGAGCAGTTACATTCCAAATCGGTGC |
| | | TAACGAAGACCAAAACTTGAGCTTGAACATCCGTAACATGAAAA |
| | | CAGACGGCGTATTAAACCTCGCAACTGCAGGACGTGTAGATGTTT |
| | | CGACTCACGCAACAGCTAAAGCTTCAGTAACAAACATCGAAGGT |
| | | GCTATCACTGAAGTATCTAAAGAACGTTCGAAACTCGGTGCTTAC |
| | | CAAAACCGTCTCGATCACACAATCAACAACCTTAAAACTTCTTCT |
| | | GAAAACCTAACAGCGGCTGAATCACGCGTTCGTGACGTTGATATG |
| | | GCTAAAGAAATGATGAACCAAACGAAAAACTCAATCCTTGCACA |
| | | GGCTGCACAAGCAATGTTGGCGCAAGCAAACCAACAACCGCAAG |
| | | GCGTTCTTCAATTACTTCGTTAA |
| flagellar hook- | flgL | >LOFDPPFF_01708 hypothetical protein |
| associated | | ATGTTAACAAAAACGAATATCGGACATTTATCAGCGAGTTATCAA |
| protein 3 FlgL | | AAGCTGAGCTCGATGCAGGAACAACTGATCAGCGGTAAAAAAAT |
| | | TCAGCGCCCGTCCGAAGATCCGGTCGTTGCGATGCAAGGCATCCG |
| | | TTACCGGACAGAAGTCCGGGAAGTGGAACAGTTCAAGAAAAACG |
| | | TCAATGAAGCGACAGGGTGGATGGATTTGACCGATTCAGCTCTCA |
| | | ATGAAGTGACATCCGCGATGAGCCGAGTCCGTGAATTGACGACA |
| | | CAAGCCGCAACGGATACATATGATGCGACCCAGCGAAAAGCCAT |
| | | CCAAAGTGAAGTTGGTCAATTGATTGAGCATATTGGTACGCTCGC |
| | | TAATACGAAATACAATGAAAAAGCGATTTTTAATGGGACTAAAA |
| | | CAGATCAACCCTTCATTTCAATGGAAAATCTGAAGGACTATTTAA |
| | | CGACATCCGGTAAATCGGTTGATACCGTCTTTACCGATGGAAACC |
| | | CTGTAACAAAAGAGAATGAAGTGATTCGCTATGAAATATCGTCCG |
| | | GCATTGAAGTTCAAGTCAATGTTTCGCCAACAAATGTGTTTAGTA |
| | | CAGAAACTTTCATGACGCTAAAAAAAGTGTATGATGCCTTAGGTG |
| | | GTACTTCGGACGCAGGTCCCGTCGACAGTTCCAACAATGGAGCTG |
| | | AACTATCGGGTATGTTGAAAGATCTTGATAGCATGCTTAATCAGA |
| | | CAGTTGAAACACGAGCCGATCTCGGGGCGCGGGTCAATCGACTT |
| | | GAGCTGAACGCTTCACGCCTTGAAGATCAAGAAATCATCGCGAA |
| | | ATCCGTCATGTCGGACAATGAAGATATTGAGGCTGAAAAGGTCAT |
| | | CATGGAATTGAAGTCATACGAAACGCTACACCGCGCGGCGCTTA |
| | | GTGCAGGGGCGCGGATCATTCAACCGACTTTGCTCGATTTCTTAC |
| | | GTTAA |
| flagellar hook- | flgK | >LOFDPPFF_01709 hypothetical protein |
| associated | | ATGGGATCAACGTTCATGGGACTTGAGACCGGACGACGTGCACT |
| protein 1 FlgK | | GACGACCAATCAGTGGGCGCTCCAGTCGACGGGAAACAATATCG |
| | | CAAATGCAGGGACAGTTGGTTTTTCGCGTCAACGACTGATTATGG |
| | | CGACGACAGAACAACTTACAATGGCAATCGGAACTGGCAAGATG |
| | | GGGCAAATTGGAACTGGTGTAAAAGGTGAGATGCTTGAACGTGT |
| | | CCGCGACGTCATGCTCGACAAACAATACCGTGATGAAGCAACGA |
| | | AGACGTCTTACTATGGCACGAAAGAAGCGGCATTTAGTCGGATG |
| | | GAAGACGTTATCAATGAGCCATCGGATACAGGACTGTCCAAAGC |
| | | GTTTGATGGTTTTTGGGAATCATTGCAGACATTGTCGACGAACCC |
| | | GCAAGACTCTGGTGCCCGCAGTGTTGTTCGTCAAAAAGCAGAGAC |
| | | GTTGACGCAGACGTTCAACTATATGGCGAAGGCGCTTAATCAAGT |
| | | ACAAGGGGACTTGAAAAGTGAAATTGAGGTCTCAACCAAAAAAG |
| | | TGAATGACTTGTTCAAAAAAATTCATAACATCAATGCCGAGATTC |
| | | ATACCGTCGAGCCGCTTGGTGTTCTGCCAAATGCCTTGTATGATG |
| | | AGCGCGACCGCTATTTTGATGAACTGTCAGAGTATGTCGATTTTG |
| | | AAAAAGTGTCTGTCGATGGCGATGCGATTCAACAAGGAACACTT |
| | | GGAAACACCGTCAAAACGGCTGAAGGCCGAATTGACGTCCGTAT |
| | | CAAACTCCCTAACGGGGATAAACTGCTGGCAGTGGATTCAGATTT |
| | | ACCACAAGCAGGAACGCTGACGTTCACAACGGACGATAAAGGTT |
| | | TGTATACAGGATTTAAAACCGATTCACAAACGATTTCATTCGACG |
| | | CGGCTGGTGGTTTTTCATCAGGACGTCTGATTGGTTTAATTGAGAT |
| | | GTACGGACATGTCGAAAATGGTCAAGCAGCAGGCGAATATGTCA |
| | | AAATGCAAGGACATCTTGATGAGATGGCGGCAACCTTTGCAACTG |
| | | CTTTTAACGAGGCACATGCTACGAATGTAAAAAAAGATAAGACG |
| | | GCAGGTACCAATGAATTTTTCGTTTCTTCAAATGGTGGGACCATT |
| | | ACAGCAAATTCAATTACACTTGGAACGGATATAAAAAAAAGTCT |
| | | GGATAATATTGCAACTTCGACTGACGGCAATATTGGTGATAGTGC |
| | | CGGTGCCTTGAAACTTGCCAACATGAAGACCGCAAGCATCCGTTT |
| | | TGAACGATCGGATACGACGACGACGATCGGTTCGTTTTATCAAAA |
| | | CGTTATTGGAGATATGGCGGTGGCAACAGATCAAGTCGCACGGCT |
| | | CGGTCAGAGTTCGGCAGTCTTGATGGAAAGTGCGGAACAACGCC |
| | | GTATGTCCGTCTCCGCTGTTTCAATTGATGAAGAGATGACAATGA |
| | | TGATTCAGTATCAACATGCATACAACGCGGCAGCGCGTAATATCA |
| | | CGACGGTTGATGAAATGCTCGATAAAATCATCAACGGTATGGGA |
| | | ATCGTAGGACGGTGA |
| flagella | flgN | >LOFDPPFF_01710 putative protein YvyG |
| synthesis | | GTGGAACTCATCAACCAGCTCACGACAACGCATATGGATCTGCTG |
| protein FlgN | | GAACTGGCACACGAAAAGAAACAGGTCCTGATTCAAAACGATAT |
| | | GCCACGCCTGTCGCAAATCGTCAAGGAAGAACTGGTTTATCTGAA |
| | | ACGGATGGAGCAGTTGGAACAGCAACGGATTGAACACATGGGAG |
| | | CGGTGACAATGACGGAATGGCTTCAAGTCCATCCGGAAGATGTC |
| | | GAGGCCATGCGCCAGTTACTTCAGGCAATCGGTAAGCTCAAAATT |
| | | ATCAATGAATTGAACGCCGACTTGCTCGAACAATCGTTACAATAC |
| | | CTCAACTGGCATCTCGAACTATTAGTGCCAGAAGCAGATGATTTT |
| | | ACATACGGTCAATCGGCGCTTGATCGCGCCCACTTCAATCGAAAC |
| | | GCCTAA |
| negative | flgM | >LOFDPPFF_01711 hypothetical protein |
| regulator of | | ATGCGAATTGATTCTACGAAATGGGTTAACATGCCTAAGACGTAC |
| flagellin | | GAAAGAAATCAAAAAGTAGAAGGAACAGAAGCAACACGTACGA |
| synthesis | | ATCGGCCGGACGAGGTGACGATCTCAAGCGAAGCGCGGATGCGT |
| FlgM | | TTCAGTGAGACAGGCACATCCCGGACCGAGAAGATTGAATCCCTT |
| | | CGCCAAGCCATTCAGGATGGAACCTATAAACCGGATGCGAAAAA |
| | | AATCGCAGAACGTTTCTTGAACTTGTAA |
| alkaline | phoA, phoB | >LOFDPPFF_02075 Alkaline phosphatase 3 |
| phosphatase | | ATGAAGTTGAAACGAATCATCCCGATTATGGCATTATCGACATTA |
| [EC:3.1.3.1] | | TCCCTTAGCACGATGATCTCGACAGATAATGCCGAGGCCAAGACG |
| | | AAATCATCCAAATCTCCGGAAATCCGTAACGTCATCTTTTTGATC |
| | | GGTGACGGAATGGGTGTTTCTTATACATCTGCTCACCGTTATCTG |
| | | AAAAACAATCCCGCTACACCTGTCGCTGAGAAAACCGCATTCGAT |
| | | CAATATCTAGTCGGTCAACAGATGACCTATCCGGAAGATCCGGAA |
| | | CAAAACGTCACCGACTCTGCTTCTGCCGCAACGGCGATGTCATCC |
| | | GGTGTCAAAACGTATAACGCGGCAATCGCTGTCGACAACGATAA |
| | | GTCGGAAGTCAAGACCGTCCTTGAAGCGGCAAAACAACGCGGCA |
| | | AATCGACGGGACTCGTCGCGACGTCCGAAATCACACACGCGACG |
| | | CCGGCCTCATTCGGGGCGCATGACGAGAACCGCAAGAATATGAA |
| | | CGCCATCGCCGACGACTATTTCAAAGAACGTGTCAACGGAAAAC |
| | | ACAAGATTGACGTTCTGCTCGGCGGCGGGAAATCGAACTTCGTCC |
| | | GTCCGGATGTTGATTTGACGAAATCGTTTAAGAAAGACGGTTACA |
| | | GCTACGTCACGGATCTTGATCAAATGCAGGCAGATAAGAACAAG |
| | | CAGGTACTTGGTCTGTTCGCGGACGGCGGACTGCCAAAACGAATC |
| | | GACCGCGAGAATACCGTCCCGTCTCTCGAGCAGATGACGAACTCG |
| | | GCCATCAAACGTCTTGATTCGAACAAAAAAGGCTTCTTCTTGATG |
| | | GTTGAAGGAAGCCAAATCGACTGGGCAGGTCACGATAACGACAT |
| | | CGTCGGAGCGATGAGCGAGATGGAAGACTTCGAACGTGCCTTCA |
| | | AAGCAGCGATCGCTTTCGCCAAAAAAGATAAACACACACTCGTC |
| | | GTCGCAACAGCCGACCATTCGACCGGTGGTTACTCGATCGGAGCA |
| | | GACGGGATCTACAACTGGTTCGCCGAGCCGATTAAAGCAGCGAA |
| | | AAAGACGCCGGACTTCATGGCGGCAAAAATCATTGAAGGTGCAG |
| | | ATGTCCGGAAGACCTTGACGACGTACATCGATCAAAACCGACTTG |
| | | CCTTGACGGAGGAAGAAATCCAGTCCGTTTCACGTGCCGCGGAGT |
| | | CGAAGAAAGTGCTCGACGTCGATAATGCGATCGAAGACATCTTC |
| | | AATAAACGTTCGCACACCGGCTGGACGACAGGTGGACACACCGG |
| | | GGAAGATGTTCCGGTCTATGCCTTCGGTCCTGCAAAGGAACGATT |
| | | CGCCGGACAAGTTGATAATACGGATCACGCCAAAATCATCTTCGA |
| | | TCTGTTGAAATCGAAGAAATAA |
| pyoverdine | pvdD | >LOFDPPFF_02108 D-alanine--D-alanyl carrier protein ligase |
| synthetase D | | ATGTCAATCACTTCACTCCGATCACCCTTATTGGAAACCCTAACT |
| | | AAAATCACAAAACAGACTCCGATGAAAGAAGTATTACTGACGGA |
| | | AGGCGCTACGTATACCTTAGAAGATATCCGGGTGCGTTCCAATGC |
| | | GATGGCACACCAACTAGAGAAATCATTTACGACACAAAGATATA |
| | | TCCCGGTTTATACGACCGACAACGTCCAATCGATCTTCAGCATGT |
| | | TGGCGTGTTGGAAAGCAGGAAAAGTCTACGTTCCATTGAACCCGC |
| | | AGACCCCTGTGCCAAAAATCCACCAACTAATGTCCACACTCCACA |
| | | GTGAGTCGATTTGGACAGATGGACTACCGGAAGAATACGATATC |
| | | CCGCAGATCATCTTTTCTAAAGAACGAATGGAACATGATGTTGAC |
| | | GTGGTGGGAACAGAGGTCGCTTATATCCTAATGACATCTGGTAGC |
| | | ACGGGCGAACCTAAGAAGGTAGAAGTAACGCACGCCAACTTGGA |
| | | TTGGTTACTGCGTACATTGGAGCAGACCATCCCATTTGCAAAGAA |
| | | CGATCGTTTTCTCGTCTCGACGCCACCTGCTTTTGATGTCGTCCTT |
| | | CATGAAATGTTGGCGTTCCTCTATGGAGAGGGACAAGTCGTCTGT |
| | | TTCCCGGCATATTCGAACATCCAAAAGATTAAGGAATTGCCAAAC |
| | | TTCGTCAAGCGTTATGACATTACGCATATCGCGTTATCACCATCTG |
| | | CCTGTACGCAAATCTTGAACCGTGAAAACGAACGCGTGAAGTTG |
| | | GCTTCTTTACGAAAAGTATTATTAGCGGGAGAGGCGCTAGGAGTA |
| | | CCGCTCGTCCAAAAACTTCATACACATTTCCCGAACGTCGACGTC |
| | | TACAATCTATATGGACCGACTGAGACGACAGTCTATGCGACTTGC |
| | | GTGAAGATTGATGATGTAGTAAATGAAGAGATACCTATCGGAAA |
| | | AGCACTTGCCGGTACTTCTATTATATTTCGTGATAAAGACGGACA |
| | | GTTGAACAATTCCGCTGGTGAGATATTGATTGGCGGGAACGGCGT |
| | | AAGTCGTGGATACCTGGGCAATCCATCATTGACAGAAGAGAAGT |
| | | TTCTGACCATTGGAGAGGCACGCTATTACGCTACAGGTGACCATG |
| | | GACGAAAGGACGAGAGCGGCATAATTTTTTACGAGGGAAGACAA |
| | | GATGATCAAGTGCAGGTGAACGGAATTCGGGTCGAACTCGGAGA |
| | | AATCAATGCAGCACTCCATGCGGTTCGTCCGACAGGTACCTTCGA |
| | | AACATTATATCTAGCGAATCGCCTAGTCGTCTTTAGCGATACTCA |
| | | TTCGTTCACGTCAGAAGATGCGCAAATGTTGAAAGAAGAACTGA |
| | | AGAAACGAATCCCTTCGTATATGATTCCGAGCATGTATGTGACCG |
| | | TACCAGAATTCAAAATGACGGCCAACCGTAAGCTGGACCGTCGTT |
| | | ATCTCGAGTCTTTCATCGAGACGACGGATATAGGAGAGCTCGTTA |
| | | AGGAGGAATCAGTTCAAAATATGGATCAACTTTTGGTGCGTGTTT |
| | | CGAATCACTACGGTCGTCCAGTCACACCAGATATGGATCTCATAC |
| | | ACGATCTTCACTTGGACTCGCTTGATCAGCTCGATCTTCTCCTCTT |
| | | GTTGGAGGATCATTTCAATATGACGCTTGTAGATGATTTTGTAGT |
| | | GACGCATCCAACGCTTCGCCGGGTTCAGATACAATTGTCACGAGA |
| | | GGAAGAGAGAGGGACGAATATCATCGAGGTTGATGAAGAATATT |
| | | GGAACGGCGTCGTCGAAGACAATATGGTGGCGAACCGGGCGCGA |
| | | TATGAACAAGTGACAAAAGAACAAAAAGAAACGTTTTATCTGCA |
| | | AAAGAGTTATCATGTCGATGGTTTCCGACAAGTGTTACACGAAGT |
| | | TGTCTCGATACCTGAGACATATGAACGGAGCCAATTGCAGGCAGC |
| | | TGTCGATGCCTTGATTGTACGCCATCCAATGTTACGGAGCTTCTTG |
| | | AAGGTGCAAGAGGATCGTTTACAGTTCACAGTGTATTCAGAAAA |
| | | GGCGTCATTCCGCTTGTTATCTGTGCCGACAGTTACTGAGGATCA |
| | | ACGACAGAACTGGATTGAAAGAATGAAACAACAGTACTTAGAGG |
| | | ACTTGATGGCATATTTCATTCATGATGTGTCTACGAACCGTATCG |
| | | AACTATTTATCAACCACCATGTCGCAGATCAGGCATCGATGAACC |
| | | TCTTGAAGCAGGATCTATCTGCCTTGCTTCAGGGCAAGGTATTAT |
| | | GTCCTTTAGCTGTCGACTATTGGGATTACATCGATTACATCGATGC |
| | | GAATATTGATCGAGCGGAGTCAGAAATCGAGCAAGTGAGTCATT |
| | | CCGGATTCGCCGACGTCACAAGCGACGCGTTTGACGTCAACGAA |
| | | GGTCGTCCGGTTCGATATCTATCGTTCCTGCTCAAGCGAGAAACA |
| | | CCGGAAGAGTATATTGCTTATGCCAATTACATCATCTTAGGTGCC |
| | | TTGGCAGCCGGACAATCACGACAACAGATGAGTGGTTCGACTATC |
| | | GTCGATCTACGATCGTTTAACGGACTCGACATTAAGGGAGTCGTT |
| | | GGAGACGTTCATACTACAATCCCACTCTGTCTTGAGCAGGGAGAG |
| | | TCGTTCGAGACATTCAACCGGAAGTTCGACAGCTGGTATAAACAG |
| | | TTCGAGTCTGGAATTAATTTTAATCATCTACTGTATCGCGACTATC |
| | | CACATGTCCAGAGTGCACATCGTACGTTCGAGCATAACTTGGATG |
| | | ACAATCTGAAAGTAAGCTCGAGCTTCCTCGGTGCAATACGAGAAC |
| | | AAGAGATTCCGACGATGTTAGAAGAACTAAAAGCTTCACATACC |
| | | ATTCTTCAGAATTTCTCGACACGTAAGCTCTATGCTTCCATCTTCT |
| | | ATACAGGAAAGCAGTTAATCATCGTCCCACTCAGTCAACCAATGT |
| | | TATCGGAAGACTTTATTACTAGACTAGGTGGTGAGATCCATCATG |
| | | AAGACGAACCGAAATAA |
| chemotaxis | motB | >LOFDPPFF_02284 Motility protein B |
| protein MotB | | ATGAAACGGAAAAAGAAGCCGCATGACGAACATATCTCCGAAGG |
| | | TTGGCTGATTCCTTATGCCGATCTTCTGACGTTATTACTGGCCTTG |
| | | TTCATCGTTCTGTTCGCTTCGAGTAACGTCGATGCCGTTAAGCTTA |
| | | AAGCAATGTCCCAATCATTCAGCTCCGTCTTTAACGGCGGTTCCG |
| | | GAATGATCACGAACAGTTCATTGTCGACCTCACAAGAAGAAGAA |
| | | GATTCAAAAAAAACGGATGCCCGAACAAAGGCGCAAAGTTATGA |
| | | AATCGCTGAACTTGAAAAAATCAAGGAAGAAGCAAATGACTACA |
| | | TCAAACAACAGAAGCTTGAAAAAGACATCAAAGTCGAAGTGACG |
| | | AATGAAGGACTGGTCTTCACGATCCGCGACCGTGCGCTCTTTTCC |
| | | CCTGCCCAGGCCGAAGTGCGTGGCAATGCCGTTCAGATTGCCCAG |
| | | GGGATGAGTAATTTACTCGTTAAAGCCGGTCAGCGCCAAATTCAG |
| | | GTGTCCGGTCATACGGACAACATTCCGATCAACACGGCACAATAT |
| | | CCGTCCAACTGGGAGCTGAGCACTGAGCGGGCGATCAGTTTCATG |
| | | CGGGCACTCCAACGTAATTCGCAGCTTGCTCCTAAACGGTTTACC |
| | | GTTAGTGGATACGGTGAATATCAACCGATCGCTTCCAACCAGACG |
| | | GAAAGCGGTCGGAGTCAAAACCGCCGTGTCGAAGTATTGATCCG |
| | | TCCGTTGATTGACATCAAAGCACAAAATGTTCTCGACGAGACGAA |
| | | AGTCACACCATCATAA |
| chemotaxis | motA | >LOFDPPFF_02285 Chemotaxis protein PomA |
| protein MotA | | ATGGATATCGTGTCGATTATTGGTATTATACTAGGCCTCATCACCC |
| | | TAGTCGGAGGAATGATTTTAAAAGGGGCTTCGCCGGTCGCCCTCT |
| | | TGAACCCGGCGGCACTTGTCATCATCTTTGCCGGAACCATTGCGG |
| | | CCATCATGATTTCATTTCCGAAAGAGCGACTCAAAATCGTTCCTG |
| | | CTTTATTTAAGGTCATCTTCTTTGAGCAAAAACTGATGACGAAAC |
| | | AAACGTTGTTGCAACAGTTTTTAACACTTTCGACACAAGCTCGAA |
| | | AAGAAGGGTTGTTGTCACTCGAAACAGCACTCGAAGAAGTGGAT |
| | | AATGCCTTCATGCGCCGTGGTGTCATGATGGTCATCGACGGACAA |
| | | CCGTCCGAATATGTCGAAGATGTCATGACCCGCGATCTCGAAAAC |
| | | ATGACAGAACGCCATCACGCCAACGCCAACATCTTTACGCAAGCT |
| | | GGTACATATGCGCCGACTCTTGGTGTACTCGGAGCCGTCATCGGA |
| | | CTCGTCGCTGCCCTGTCCGACCTGTCGGACATCGAAAAATTGGGC |
| | | CATGCGATTTCCGGTGCGTTCATCGCAACCCTGTTCGGGATTTTCA |
| | | CGGGATATGTCCTCTGGTTCCCGTTCGCTACCAAACTCAAGCAAA |
| | | AATCAGCCAATGAAATCCAACTGTATGAAATGATGATCGAAGGG |
| | | ATTTTATCGATTCAGAATGGAGAGTCCCCTAAAAATCTGGAGGAT |
| | | AAGCTACTTGTGTATCTGACACCGAAGGAGCGTGCGACGTATGAA |
| | | ACGGAAAAAGAAGCCGCATGA |
| flagellar hook- | flgK | >LOFDPPFF_02407 Flagellar basal-body rod protein FlgG |
| associated | | ATGCAATCACTTTATACATCAGCCAGCACGATGGCTCAGCTCCAG |
| protein 1 FlgK | | AAGCAGCTCGATACGACGGGACATAATCTGGCGAATGCCAATAC |
| | | GAACGGCTATAAACGTCGGGACTCTCAGTTTAATGAACTGTTGGT |
| | | CCGCAATCTCAACAATCAGCCGGGCGGTCTTGTGACCGGACCGTT |
| | | GACGACACCGGAAGGGCTGCGGCTCGGCGTCGGTGGATATGTCG |
| | | CCAATGAAGCGACACGGTTTACGACAGGGACGTTCCAGAATACG |
| | | GGACGGAAACTCGATGCTGCGCTCAGCAATCCACATCATTTCTTT |
| | | GGTGTGATTGATGCGGACGGTGTGACGAAATTCACGCGGGACGG |
| | | CAATTTTGAATTGTCACCGCAAGCGAACGGACAAGTTCTGTTGAC |
| | | GGATGATGCCGGACGTTCTGTCATCAATCAGGCGAATGAGCCGAT |
| | | TACGTTTCCGGATACGGCGACATCGATTGAACTGAACAAAGACG |
| | | GCAACATCACGGGCATCTTGAACGGGGAACGACGGGTGCTCGCC |
| | | CGGATTGGCGTCGCCGATATTCCGAACCACGGCGAATTGACGGAT |
| | | GTTGGTGCCGGACTCTTCACGGCGACGGGACAATACCAGAATGC |
| | | GGCAGGAAATCCGTTGACGGTCGGCACACTCGAGACGTCAAACG |
| | | TCGACATGGGGACGGAAATGACGAACTTGACGCAGATTCAGCGG |
| | | GCCTACCAGTTCAATTCCAAAGCCTTGACGACATCGGATCAGATG |
| | | ATGGGAATCGTGACGTCGCTTAAGTAA |
| iron(III)- | yfmD | >LOFDPPFF_02450 putative siderophore transport system permease |
| citrate import | | protein YfhA |
| ABC | | ATGAACAGACTTCGTCAAAAACCATGGCTCGGTCTAGTCCTCATG |
| transporter, | | TCACTCCTTGTCACCGTGCTCAGTTTCCTGTTGCTTGGCATCGGTT |
| permease | | CCGTGTTTCTGAAGCCGGGTGAAATCGTCGCGGCTCTTCAAGGAG |
| protein | | ACGGCGCCGGTTCCTTCATCGTCTGGAACTACCGGTTGCCGCGGA |
| | | CGCTCCTCGCGTTACTCGCCGGCGGTTGTTTTGCCTTGTCCGGTGT |
| | | CTTGTTACAGGCGATTATCCGCAATCCGCTCGTCTCACCGGATGT |
| | | CATCGGGGTGACGAATGGGGCCGCGTTGTTCGCCGTCTTGACGAT |
| | | TGCTTTGATTCCTGATGGTCCGCTTGTTTTGACACCGATTGCCGCC |
| | | TTAATAGGAGCAACGCTTGTGATGGTCGCGTTGATGCTGCTGGCG |
| | | GATCACGGGAAACTGCAAAACAGTTCCTTTGCCTTGCTCGGCATC |
| | | GCCGTCAGTGCAATCTGTGCATCGGGAACGGAATACCTGTTGATC |
| | | AAGTTTCCTCTCCAGACCAATGATTCGCTCGTCTGGCTCGCCGGC |
| | | AGCATGTTCGGCAAAGGTTGGACGGAAGTGTACGTCCTGGCACC |
| | | GGTCTTCCTGTTGCTTGGACTCGTCATCTGGTCCGGTCACCGGCAA |
| | | CTCGACATCTTATCACTCAGTGAAGACGCAGCGATTGGTCTCGGA |
| | | TTACGGATGAAGGGAACACGGTATGTGTTCCTCGCCTTTGCCGTC |
| | | GCTTTGGCAGGTGTTGCGGTCGCAATGGTCGGGTCAATCGGTTTT |
| | | CTTGGCCTCGTCGCCCCGCATATGGCACGGCGCTTGATCGGACAC |
| | | CGGCATCATCTGTTGATTCCGATGGCTGTCCTCGTTGGGGGTGGA |
| | | CTGCTTGTCGTTGCGGACGCGCTCGGACGCGGCATCCATCCGCCG |
| | | CTTGAGATTCCGGCCGGATTAATCACGGCAATCATCGGTGTGCCG |
| | | TACTTCCTGTATCTGTTGCGAAAAGAACGGGCGTGA |
| iron(III)- | yfmE | >LOFDPPFF_02451 putative siderophore transport system permease |
| citrate import | | protein YfiZ |
| ABC | | ATGATCCGACACATACGATTGATCCGCTTGCTCGTCGTACTCCTTG |
| transporter, | | TCCTGATCGGACTCGGATCCTACCTCAGTCTGTTTCTCGGCGTCAC |
| permease | | GACGATTCAACCGCTTGAAGCCATTCGGGAATGGTCATCCGGCAA |
| protein | | CTTGTCGAAAGAGACACTGGTCTTGACGACACTCCGCTTGCCGCG |
| | | GTTGTTACTCGGTTTATTACTCGGGGCAAACTTAGCCGTCGCCGG |
| | | TGCCTTGATGCAGGCGGTCACACGTAATCCGTTGGCTTCGCCGCA |
| | | AGTGTTCGGCGTCAACGCCGGGGCGTCGCTGTTTGTCGTCCTCGC |
| | | TTTGTTGTTGTTTCCGGCACTTGGGACAGCGAATCTGGTCTATTTC |
| | | GCCTTTTTCGGTGCAATGGTTGGCGGATTACTGGTCTTTTCGTTCG |
| | | CCTCTGTCCGCGGCATGACGAGTCTGAAACTGGCCCTCGTCGGGA |
| | | TGGCGATCCACTTGTTGCTGACGTCCTTGACGAAAGGGTTGATTT |
| | | TGTTCAACGACCGGATCACCAACGTCCTGTACTGGTTATCCGGTT |
| | | CAATCAGTGACAGTGGATGGATCGAAGTCCGGTTGATTTTACCCT |
| | | GGTCGATCATCGGCTTGATCTTAGCCTTCAGCCTGGCCAAATCGC |
| | | TGGCGATTTTCCAACTCGGTCAAGATGTGGCCGTCGGACTGGGGC |
| | | AGAACATCACCCGGATTCGGATGCTGGCAGCCGTCGCTGTTGTCC |
| | | TGCTGGCCGGGGTGACGGTCGCGGTCGCCGGAGCAATCGGCTTCA |
| | | TCGGTCTGATGGTCCCGCATATCGTCCGGCGATTGGTCGGTGAGG |
| | | ATTACCGCTATGTCTTACCGATTTCGGCGTTGTGCGGTGGTCTGTT |
| | | GCTGACATATGCTGATGTCCTCGCCCGGTTCATCGCCTATCCGTAT |
| | | GAATCACCGGTCGGGATCGTGACCGCGTTACTCGGAGCGCCGTTC |
| | | TTTTTGTATTTAGCGAAACGGCAGACAAGGGGGATTGCCTAA |
| iron(III)- | yfmC | >LOFDPPFF_02452 Fe(3+)-citrate-binding protein YfmC |
| citrate import | | ATGTCACGTACACGCACATCATGGGCATTAGCCGTCTTGATGGTC |
| ABC | | AGTTGTCTGATGCTTGCGGCATGCGCCGGACAAGCAAAAGAAGA |
| transporter, | | GACGAAACAGACGCATAAAGTCACGCACGAAGCAGGGACAACA |
| iron(III)- | | AACGTTCCGGACAATCCGAAACGCGTCGTCGCCCTGGAATTCTCA |
| citrate-binding | | TTCGTCGACGCGCTTGACGAACTGGGGATCGAACCGGTCGGCATC |
| protein | | GCCCAAGAAAACAAAGACGATGTGTCGGGTCTGCTCGGCAAGAA |
| | | GATTTCCTTTACGGAAGTCGGAACACGCCAGCAACCGAATCTCGA |
| | | AGTCATCAGTTCGCTGAAACCGGACTTGATCATCGGTGACTTCAA |
| | | CCGGCATAAAGGAATCTACAAACAGTTGCAGCAAATCGCACCGA |
| | | CGATCATTTTAAAGAGCCGGAACGCGACGTATCAGGAAAACATC |
| | | GCGTCGTTTAAGAGCATCGCGGAAGCGGTCGGTCAGACGGATAA |
| | | GATGGATCAACGTCTCGAGTTACACGAAGAGCGTCTCGCAACAG |
| | | CCAAACAAAAAGTCGATCCGAACGATCAACGTCAGATTATGGTC |
| | | GGTGTCTTCCGGTCAGATTCGTTGACGGCACATGGCGAAACATCG |
| | | TTTGACGGCGAATTGCTCGAAAAGATGGGGATTGATAATGCCATC |
| | | ACGAAAACAGCGGAACCAACCGTGACGATCACACTGGAACAGAT |
| | | CGTCAAATGGGATCCGGATGTCATCTTCATGGCAGAAGCCGATCC |
| | | GAAGTTGCTTGATGAGTGGAAAAAGAATCCGCTGTGGAATCAAA |
| | | TCACAGCCGTCAAAAAAGGGGAAGTCTACGAAGTCAATCGTGAC |
| | | TTATGGACCCGTTACCGTGGACTCGACGCTGCGGAACAAATCGTC |
| | | GATGAAGCCATTCAACTGCTGAATCAAACAAACAAGTAA |
| spermidine | speE, SRM | >LOFDPPFF_02511 Polyamine aminopropyltransferase |
| synthase | | ATGGAACAAAAATTAAAGTTATGGTTCACGGAACACCAAACGGA |
| [EC:2.5.1.16] | | AGATTACGGTATCACATTCCGTGTCAACCACGTTTATGAGAGCGA |
| | | ACAAACGGAGTTTCAACGCCTGGAGATGGTTGAGACGGACGAGT |
| | | TCGGTACGATGTTGTTACTTGACGGAATGGTCATGACAACAGATC |
| | | GAGATGAGTTTGTATATCACGAAATGGTCGCACACGTTCCACTGT |
| | | TCACACACCCGAATCCAAAATCGGTTCTGGTTGTTGGTGGAGGAG |
| | | ACGGCGGCGTCATCCGCGAAGTGTTAAAACACCCATCAGTCGAA |
| | | AAGGCTGTTCTTGTCGAAATCGACGGAAAAGTCATCGAATACTCG |
| | | AAGAAATATCTACCAAACATCGCAGGTGGTCTCGACGACGCACG |
| | | TGTTGAAGTCATCGTGGGAGACGGCTTCATGCATATCGCAGAAGC |
| | | AGTCAATGAATATGATGTCATCATGGTTGACTCGACAGAACCTGT |
| | | TGGTCCTGCCGTTAACCTGTTTACAAAAGGTTTCTACTCAGGAAT |
| | | CTCAAAAGCATTAAAAGAAGACGGCATCTTCGTCGCACAGTCGG |
| | | ATAACCCATGGTTCACACCAGACTTAATCCGTGACGTCCAACGCG |
| | | ATGTCAAAGAAATCTTCCCAATCACGAAACTCTACATTGCCAACG |
| | | TTCCGACTTACCCGAGCGGTCTGTGGACATTCACGATCGGATCGA |
| | | AAAAACATGATCCTCTCGCTGTCGCACCAGAGCGTTTCCACGAGA |
| | | TCGAAACGAAGTACTATACACCGGAACTTCACACAGCAGCATTCG |
| | | CGCTACCGAAGTTCGTCAAAGATTTAACGATTTAA |
| agmatinase | speB | >LOFDPPFF_02512 Agmatinase |
| [EC:3.5.3.11] | | ATGCGTTTTGATGAAGCTTATTCAGGTAAGGTATTTATCGCGAGT |
| | | CAACCGACTCACGAAGATGCAAAAGGTGTCTTGTACGGCATGCC |
| | | GATGGACTGGACGGTCAGTTTCCGTCCCGGGTCACGATTTGGTCC |
| | | GGCCCGGATCCGTGAAGTGTCACTCGGACTCGAAGAATACAGTCC |
| | | GTATCTCGACGGTGACATTGCGGATGCGAAATTGTTTGATGCCGG |
| | | CGATATCCCGTTGCCGTTCGGCAATGCCCAAAAGTCACTCGACAT |
| | | GATCGAAGAATACGTCGATTCGTTGCTGACGGCAGGAAAGTTTCC |
| | | GCTTGGTATGGGGGGCGAACACCTCGTCACATGGCCGGTCGTCAA |
| | | AGCCTTTGACAAACATTATGATGACTTTGTTGTGCTGCACTTTGAT |
| | | GCACATACGGACTTACGCGATTCGTATGAAGGAGAACCGTTGTCG |
| | | CACTCGACACCACTTAAAAAAATCGCAAACTTGATCGGACCGGA |
| | | AAACTGTTATTCATTCGGCATTCGTTCAGGGATGAAAGAAGAGTT |
| | | TGAATGGGCGAAGACGTCCGGTTACAACTTGTTTAAATACGAAAT |
| | | CGTCGAACCGTTAAAAGCTATTTTACCGAAGCTTGCCGGTAAAAA |
| | | GGTTTACGTGACGATCGATATCGATGTGCTCGATCCTTCGGCGGC |
| | | ACCCGGAACCGGGACGCAGGAAATCGGTGGTGTGACGACAAAAG |
| | | AATTACTTGAAGTCGTTCATATGATTGCACGTGCGGATGTCGACG |
| | | TCATTGGAGCCGATTTGGTTGAAGTCTGTCCGGCGTATGATCAGT |
| | | CTGACATGACAGCGATTGCGGCTGCCAAAGTCTTACGTGAAATGA |
| | | TGATTGGGTTTATCAAGTAA |
| acetolactate | ilvB, | >LOFDPPFF_02646 Acetolactate synthase |
| synthase | ilvG, ilvI | ATGACAGAAAAACAAAACAGTGAAAAAGAAAATATGGTACAGA |
| I/II/III large | | AGAAAACAGGTGCCGATTTAGTCGTCGATACACTGATTGAACAA |
| subunit | | GGGGTCGACTATATTTTTGGTATTCCGGGAGCAAAGATTGACTCC |
| [EC:2.2.1.6] | | GTCTTCAACGTCCTTCAAGATCGTGGACCGGAATTGATTGTCGCA |
| | | CGCCACGAACAAAACGCCGCCTTCATGGCACAAGCCATCGGCCG |
| | | CTTGACTGATAAACCGGGTGTGGTCCTCGTAACTTCCGGACCAGG |
| | | GGCTTCGAACCTCGCAACCGGACTTGTGACGGCCAATTCGGAAGG |
| | | TGACCCCGTTGTCGCGATTGCCGGTGCCGTGACACGTGCCGATCG |
| | | TTTGAAACGGACCCATCAATCGATGGACAATCAAGCGCTCTTCAC |
| | | ACCAATCACGAATTTCAGTGCCGAAGTTCAAGATGCAGACAACAT |
| | | CCCGGAAGTCCTTTCGAATGCATTCCGGACTGCCGAAACAACATC |
| | | CGGCGCTGCTTTCGTCAGCATTCCGCAAGACGTTGGTCTCAGTGA |
| | | ATCAAACGTCACTTCCTTTAAAGCGGTCCCAACACCAAAACTCGG |
| | | CATCGCACCCGAAGAATGGATCAACGAGACAGCAAATCTGATCG |
| | | AAAAAGCACAATTGCCGGTCTTGTTACTCGGGATGCGTTCCAGTC |
| | | AGCCCCATGTCGTCAAAGCCATTCGTGCACTGTTGAAACGCGTTT |
| | | CGATTCCGGTCGTCCAGACATTCCAGGCAGCCGGAACATTGTCAC |
| | | GGGAGCTCGAGTCGAATTTCTACGGACGTGTCGGTTTGTTCCGCA |
| | | ATCAACCGGGAGATGCCTTGCTCGCTGAAGCCGATCTCGTGTTAG |
| | | CTGTCGGATATGATCCGATCGGTTACGATCCGAAGTTCTGGAATC |
| | | AACCTTCACACGAACGGACTTTGATTCACTTGGATCAAATGCGGG |
| | | CTGAAATCGACCATTTCTATCGTCCGGATCGGGAACTTGTCGGGG |
| | | ATGTCGCTGCAACAATCGACGCATTGGCTGATCGACTCAATCCGC |
| | | TGAGCCTGCCAACCAGCTCGACGGAATTTTTACGCGGTTTACAAC |
| | | AACGCCTCGAGGAGCGAGATATTCCACCGATTGTCAAGGATTCAC |
| | | CGTTAACACATCCGCTGTATTTCATGAAAACCTTACGGGAACAAA |
| | | TCGCTGACGATGTGACGGTTACGGTCGACGTCGGTTCGCACTACA |
| | | TCTGGATGGCACGTCATTTCCGGTCTTACGAACCGCGTCACTTACT |
| | | GTTCAGTAACGGGATGCAGACGTTAGGTGTCGCATTACCTTGGGC |
| | | GATTGCCGCAACACTGGTTCGTCCAGGCAAAAAAGCCGTCTCGAT |
| | | CTCAGGTGATGGTGGTTTCCTCTTCTCGGCGATGGAGCTTGAGAC |
| | | AGCCGTCCGTTTGAATGCCCCGCTCGTCCATTTCGTTTGGCGCGAC |
| | | AGCGGCTTTGATATGGTGGCTTTCCAACAAGAGATGAAATACAAA |
| | | CGAAAATCCGGCACGTCGTTCGGTGAAGTGGATCTTGTGAAATAT |
| | | GCTGAAAGCTTTGGTGCAAAAGGCTTGCGTGTCAATCATCCGTCT |
| | | GAACTCGTCGCCGTCATGGAAGAAGCATGGCAAACAGAAGGTCC |
| | | GGTCATCGTTGATGTTCCAATCGATTACAGCGATAACATTACACT |
| | | CGGTAAAGAAGTACACTTGGATCAACTCAACTGA |
| acetolactate | alsD, | >LOFDPPFF_02647 Alpha-acetolactate decarboxylase |
| decarboxylase | budA, aldC | ATGCAGCGTGAGGACACCTTACTTCAAATCTCGACGATGATGTCT |
| [EC:4.1.1.5] | | TTGCTCGATGGTGTTTTTGAGAGCGAAACAAGTTATGCCTCCATT |
| | | CTCGAAGGACATGACTTTGGAATCGGAACGTTTGATCACCTCGAT |
| | | GGTGAAATGATTGGTTTTGACGGTTCCTTCTACCAACTCCGGTCA |
| | | GACGGCAGCGCACGTCCTCTTGATCCGGAGACGACCACTCCCTTT |
| | | TGTTCACTGACACGGTTCACGCCGGAACAGACGCTGTCCGTTGAT |
| | | CAGGAAATGACAAAACAGGATTTTGAACAATGGTTGAGTGAACA |
| | | ACTCGGTACAATCAACAGTTTTTATGCTGTCCGGATCGAAGGACA |
| | | GTTTAGTGAAGTCAAGACACGGACGGTCGCCCGCCAAGAAAAAC |
| | | CGTTCCGTCCGATTACGGAAGCCGTGGCCACGCAAAGTGCCCGGA |
| | | CGTTCGAGCAGACGGAAGGCACACTGGCCGGCTATTACACGCCC |
| | | CGGTTTGGTCACGGTATCGCAGTCGCCGGTTATCATCTCCACTTTA |
| | | TCGACAAGGAACGAAGTGGCGGTGGCCACGTGTTTGACTATACG |
| | | GTCAATCGCGTGACGGTCACGTTCGAAGAGAAACCGCGGCTCGA |
| | | TCTTCGACTGCCGACGACAACTGCTTACCGCGAGGCGGATCTTGA |
| | | AAGTCACGATATCGAACAAGAAATCAAAATCGCAGAAGGTTAA |
| pyrroloquinoline | pqqG | >LOFDPPFF_02669 hypothetical protein |
| quinone | | ATGGCGTTACTGCTTGCGCATCATCGTCCTGTCGCCCGGACCGTTT |
| biosynthesis | | CCTGGGCCGGGGTGACAAACCTTGTCTGGACGTATGAAGAGCAG |
| protein G | | CAGACGATGCGGAAGATGTTGCGACGCTTCACGGGTGGGTTGCC |
| | | GGAGCAACAAAAAGAAGCTTATCAAGTTCGTTCTCCGCTCTATTT |
| | | TCCGCCGCAAGGCGATGTCTTGTTGATTCACGGCTTGTACGATCA |
| | | AAATGTCCGATTGCGTCATGCGACGAATTACGCCGCCCGTTATCC |
| | | AAAGCAGACGCATTTAAAAGTGTATCAATATGCCCATCAATTCCC |
| | | GATTCGTCAAAAATTCGAAGTGACGGATGATGTCATCAACTGGAT |
| | | GATGACGTGA |
| arginine | speA | >LOFDPPFF_02774 Arginine decarboxylase |
| decarboxylase | | ATGAAAGGTCGGATGCCGATTGTTGAAGCACTGTATGCTCATGTA |
| [EC:4.1.1.19] | | GAAAGGAAAGCTACTTCTTGGCACGTTCCCGGTCATAAAAACGG |
| | | AACAGTACTAAACGGCTTACCTTCTTTTTTAGAATGGGATAAAAC |
| | | AGAGTTGACTGGTTTAGATGATTTTCATCATCCTGAAGAAGCCAT |
| | | CTACGAAGCAAAACTTTTGTTGCGTCAAATCTATGATGCCTCGGA |
| | | TAGTCATTTTCTGGTGAATGGATCAACTGTCGGGAATTGGGCGAT |
| | | GTTAGCGGCGGTTGCGAGTCGTGGAGACCGGATCTATGTGCAACG |
| | | GAACTCGCATAAGTCTGTTTTTAATGCGTTGGAATGGTTGGGGTT |
| | | ATCGCCCGTTTTAATGGAACCGGACTACCATGCAACAGGAATCAG |
| | | CGGAAATGTTTCACGTGAAACATTAGAAGAGGCCTTGAAGCTGTA |
| | | TCCTGGAGGAGTGGCAGTCTTTTTGACGTCACCCACCTATTATGG |
| | | AGAAAGCGCCGAGATTGATAAATGGGTTCATTTGACGAAGTCGC |
| | | ACGGGTTACCCTTACTCGTCGATGAAGCACATGGTGCACATTTTG |
| | | GGGAAGCTTTTGGAGTTCGTTCTGCCTTTGAGTTAGGTGCAACCG |
| | | CTGTTGTTCAATCTGCCCATAAGACTTTACCTGCATTGACGATGG |
| | | GAGCTTGGATCCATGAACGTTTCACGGATGACGAACGAAGACGA |
| | | CTAACACGAGCGCTTCAAGCCTTTCAAACGTCCAGTCCGTCCTAC |
| | | TTGCTGATGGCTTCGCTTGATTTTGCACGTGATTACCGTCAACAGT |
| | | TTACGGTTGAACAGATGTTGGAAATACGGAACAGCCATGAACAC |
| | | TTTCACCAACGACTCAATCAACACACTGAGTTGGACGTATTTACG |
| | | TTTGATGATTGGTCGCGGATGATTGTGTCCTGCCGCGGGTATTCC |
| | | GGGAATCAAGTGTTGGCAGCATTGGCTAAGCAGGGTATAGATGC |
| | | AGAGTTTGCTCTCGGGGAACATGTCGTTTGTATTTTACCGTTGCGC |
| | | CTCTTACCGGAATCCGAATGTCATGCATGGATCGAACAAATCAAA |
| | | CAGGCACTCGATATGATGAAAACAGAAGGAATTCCCGATAAAAG |
| | | GTATATGGAACAACCTATTATGGGTAAAATAAAGGTATCATCACT |
| | | TGCTTGTCCACTCGATCAACTGGAACGTACCGTGGCAGTCGAACG |
| | | GTCTTTTGAAGAGGCAGTCGATTCTGTTTCACTTGAGACAATCATT |
| | | CCATATCCTCCTGGAGTTCCACTTTTACTACGGGGTGAACGGGTG |
| | | ACGCAAGCACATATCGAAATGATTAAGCAGTATACGACCACATC |
| | | CGTCCATCTCCAAGGTGGGGAGCTTCTATATGAAGGGAAATTGCG |
| | | TGTGATACAGGAAGGAATTACAGAATGA |
| alkaline | phoA, phoB | >LOFDPPFF_02955 Alkaline phosphatase 4 |
| phosphatase | | ATGAAGAAAACATGGATCACGACAAGCGTACTGGGAATGACATT |
| [EC:3.1.3.1] | | AGTGGCAGGTGTCACGGCGTATACATATGAAGCACCACGACACG |
| | | TCGAGGCAAAACCACAGACGGAGTCGAAGAAAAAGGTCAAAAAT |
| | | GTCATCATGATGATTCCGGACGGTTATTCTGCGTCATATGCAACA |
| | | AATTATCGTTGGTACAACGGCGGTGACGAGACAGAACTGGATCG |
| | | TCAGCTCAAGGGCATGATGCGGACATATTCCGCGAGTTCTAAAGT |
| | | AACGGACTCTGCTGCGGCAGGAACGGCGATGGCAACGGGCACAA |
| | | AAACGAACAACGGCACGATCGGGATGAATCCAAGTGGACAGGAA |
| | | GTTGAATCAATCTTTGACCGGGCGGACCGTGTCGGCAAATCAACG |
| | | GGACTGGTAGCGACATCTGCCATCACACATGCGACGCCGGCTGTC |
| | | TTCGCTTCCCACGTCGCTTCACGTGCCAACGAAGCAGACATCGCA |
| | | AAACAATACATGGATGAGATGAAAGTAGATGTCTTGCTTGGCGG |
| | | CGGTCAAAAGTATTTCTTTGATAAGGAAAATGGTGGCGTACAAGA |
| | | AGCAGGAAACCTCGTCAAAAAAGCGGAACGCGCCGGATATCAAT |
| | | ATGCCGACTCGCTGGAAAGTCTTCAGGAGACCGATGGGCGAAAA |
| | | GTGCTCGGCTTGTTTGCCGAGGAAGGAATGGCACCGGAACTCGAT |
| | | CGAGAATTGACGCAACAACCAAGTTTGTCGACGATGACAAAAAA |
| | | AGCAATTCAAACGCTGAATAAAGATAAAGAAGGATTTTTCCTGAT |
| | | GGTGGAAGGCAGTCAGATTGATTGGGCGGGTCATGCACATGATG |
| | | CTGCCTGGGCGATGAAGGATTCGGACGCCTTCCACAAAGCCGTAA |
| | | AAGAAGCGATGCGTTTTGCGGCAAAAGATAAAAACACATTGGTC |
| | | GTCGTAGCAGGCGATCATGAAACAGGCGGGATGACGGTTGGCGG |
| | | TTATGATGAGTATGTGGCAAAGCCGGAAGTCTTGAAGAACGTCA |
| | | AAGCAACCGGTGACCAGATGGTCCGTCAATTTAATGATGATTTGA |
| | | CGAATATCGCGGAAATCGTCAAACAAGAAACATCATTTTATTTGA |
| | | CTGCTCAAGAAGTCAAGACGCTCCAAACTGCTGATTCTAAAAAAC |
| | | GTGTCATGCTGTTGAATGAAATGATCAGTAAGCGGGCCTATGTCG |
| | | GTTGGACGACAACTGTTCATACAGGTGTAGATGTTCCGTTGTATG |
| | | CATACGGTCCCCACAGCGATCAGTTTGCCGGTTTACATGACAATA |
| | | CGGACTTACCCGGTTTAATTGCCAATGCGATGAAACTCAAGAAAT |
| | | AA |
| acetolactate | ilvB, | >LOFDPPFF_02961 Acetolactate synthase |
| synthase | ilvG, ilvI | ATGAACGCCGCTGAACGGTTCGTTGACTGTCTCGAAGCAGAAGGT |
| I/II/III large | | GTGACACACATTTTCGGTGTGCCGGGGGAAGAGAACATTACGTTA |
| subunit | | CTCGAAGCGATCAGTAAATCAGACATCACCTTCGTGACGACACGT |
| [EC:2.2.1.6] | | CATGAAACAAATGCGGCATTCATGGCCTCGATGTTCGGCCGATTG |
| | | AGCGGACGCCCCGGCGTCTGCCTTTCGACGCTCGGACCCGGGGCA |
| | | ACGAATATGATGACCGGTATCGCCAGTGCGACGATGGATCATTCT |
| | | CCGGTCGTCGCGATTACTGGGCAAGGGGCGACGTGGCGGCAGCA |
| | | TAAAGCTTCGCATCAGATGTTCGATCTGGTTGAGATGTACCAGCC |
| | | GATCACAAAATCCAGCACATCGATCTCTTCCGGTGAAGTCATTTC |
| | | AGAAGTCGTCCGCCAGGCGTTTGCTCAAGCTGCATCCGAAAAACC |
| | | GGGCGCGACCCACATCTCCTTCCCGGAAGACATCGCTAAAGCTGA |
| | | CGTCGATGCCAAAACTCCTTTGCTTGTGGATAGTGCTCCAACGTA |
| | | TCTGCATCCGACCTCGGTCAAGGACAGTGAAGTCTTGCGTCAAAT |
| | | GGAACAAGCGGAAAAACCTGTCGTGATTGCGGGATTCGGGATTA |
| | | ATCGGAGCGGAGCGACGGATGCCTTTCGTCACTTCGTCGAACGTT |
| | | TAGGTGCCCCCGTCGTCGAGACGATGATGGGAAAAGGAACGATT |
| | | GCGTCCGATCATGAACTCGCTGCTCACACGATCGGTCTGCCAAAC |
| | | GCTGATTATAATCAACGCATCATCGACCAAAGCGATTTAATCATT |
| | | GCGATTGGTTATGACATTACGGAACTGCCGCCGTCGAAATGGAAC |
| | | CCGAACCGGACACCGGTCCTCCACATCGACACGAATCAACATGA |
| | | GGTGGACCAATATTATCCGGTCGTGGCCAATTTGATTGGTTCTTTG |
| | | CCGGATACGTTACGGTTACTTGCCGAGGACGTGCCGAATCGTGCC |
| | | TGGTCCGGTTGGCAACAAGACCGCGATCGATTACGAAAGGAAAT |
| | | TCAGGCACCCTACTCGATGGCACTGCCGCTTCATCCTCAAAGCAT |
| | | CGTCCGGGAACTGGAAAAGGCGACCGGTTCGGACGGGATGGTCT |
| | | TTTCCGATGTCGGCGCGCACAAAGTTTGGCTCGGACGGCATTTTC |
| | | AAACGACACGCCCGAATCAATTGTTCATTTCGAACGGCTTTTCCT |
| | | CGATGGGGTACGGGTTATCAAGTGCCATCGCCGCGAAACTGCTTT |
| | | ATCCGGACCGGCGTGTCCTCTGTGCGTCAGGTGACGGGGCCTTTT |
| | | TAATGAATGGTCAGGATCTTGAGACGGCTGTTCGGCTGAAATTGC |
| | | CGATCGTCGTCATCATCTGGCGCGACGGGACGTATGGACTGATCG |
| | | AATGGAAACAACAGCAAGCTTACGGACGGGCCCCTTATATCGAA |
| | | TTCGACAATCCGGATCTCGTTCAACTCGCTGTCGCCTTTGGCGCAC |
| | | TTGGTCTGCGTGTCGGAGAACACGGCACACTGGCTGCTTGTCTCG |
| | | AGCAAGCTTTCTTGAGTGACGGACCCGTTTTAATTGACTGTCCGG |
| | | TCGATTACAGGGAGAATCTGAAGTTAAGTGACCGTTTACGAACTT |
| | | ATGGAGGATGA |
| isochorismate | pchB | >LOFDPPFF_03070 Protein AroA(G) |
| pyruvate lyase | | ATGGATCAACATGCAGAATTAAAACGCTTACGTGATGAACTCGAC |
| [EC:4.2.99.21] | | CTGGTAAACGCAGAATTACTTGGATTAATCAATAAACGGGGCGA |
| | | GATAGCGGTTGAAATCGGTAAAGTAAAACGGGCGCAAGGAATCG |
| | | ATCGGTATGATCCGGTTCGTGAACGCCAGATGCTTGAATCGATTG |
| | | CAGCAAGTAATCACGGTCCATTTGAAACAGGACGCCTACAGCAC |
| | | GTATTTAAAGAAATTTTCAAAGCCTCTTTGGAACTGCAAGGAGAA |
| | | GACCGGACACGCAAGTTGCTTGTGTCGCGTAAACAAAAGCCGAC |
| | | GAATACCATCATCCGGATCGGAGATGACGTCATCGGTGATGGCTC |
| | | GCAGCAGTTAATTGCCGGTCCTTGTGCCGTCGAAAGTGAGGAGCA |
| | | GGTCTTTGAAGTGGCCGAGCAACTCGCGAAGCATGGGGTACGCTT |
| | | CATGCGTGGTGGAGCTTACAAACCACGGACATCACCATACGATTT |
| | | CCAGGGTCTCGGTTTAGAAGGATTGAAGATGCTAAAAAAGGCAG |
| | | CTGATGCCCATGGTCTTCATGTCATTACAGAAATCATGACGCCGA |
| | | GTGCAGTTGAGTCGGCTTTACCATACGTGGACATCATTCAAGTCG |
| | | GTGCCCGCAATATGCAAAACTTCGATCTTTTGAAAGAAGTCGGCC |
| | | GAACGGACAAACCGGTTCTGTTGAAACGTGGTTTGTCAGCGACGC |
| | | TCGAAGAATTCATGTATGCAGCAGAGTACATCATGGCAAGCGGG |
| | | AATGAGCAGGTCATCTTGTGTGAACGTGGTATTCGGACGTACGAG |
| | | CGTGCGACACGAAACACATTGGATATCTCAGCTGTTCCGATTTTG |
| | | AAGCAAGAAACACATTTGCCTGTCATGGTCGATGTAACGCATTCA |
| | | ACGGGTCGTAAAGACCTGCTCCTGCCGACAGCGAAAGCGGCATA |
| | | CGCAATCGGTGCAGATGCCGTCATGGTAGAAGTTCATCCGTTCCC |
| | | TGCGCTTGCGCTCTCGGATGCGAACCAACAATTAGATTTTAATGA |
| | | GTTTGATTCGTTCATCGAGAATCTGACTTCCACTTTTCCGGCACTA |
| | | AACGTTCAATGA |
| two- | phoR | >LOFDPPFF_03681 Alkaline phosphatase synthesis sensor protein |
| component | | PhoR |
| system, OmpR | | ATGAATTTGCTAGACAATGCGATCCGTTATACGGAAACGGGCACG |
| family, | | ATTCAGGTGCAGCTCAGGCAAGAGCCTAGCCATGTGGTTACGATC |
| phosphate | | GTGGAAGACAGCGGGATCGGCATTCCCCCAGAGGAATTGCCTTCT |
| regulon sensor | | ATTTTTGAGCGCTTTTATCGGGTGGAAAAATCGCGTTCCCGAGAA |
| histidine | | CATGGAGGTACCGGGTTAGGACTTGCTATCGTCAAGCAGCTGGTG |
| kinase PhoR | | GACATGCAGGGCGGAACGATTCAAGTATCCAGCGTGTTAGGAAA |
| [EC:2.7.13.3] | | AGGCACTCGTTTCGAAATTACACTTCCGATTGGAGGGGATAGCCA |
| | | GTGA |
| two- | cheB | >LOFDPPFF_03931 Protein-glutamate methylesterase/protein- |
| component | | glutamine glutaminase |
| system, | | GTGGATGTTCTTTTTGAATCGCTGCTTCCGCTCAAGGAGTTGAAG |
| chemotaxis | | CGTCATATCGTGATCATGACCGGCATGGGATCGGACGGCGCCAA |
| family, | | AGGGATGCTGGCTCTGAAAGAATCGGGAGCTGTAACCACGATTG |
| protein- | | CCGAATCGGAAGAAACCTGTATCGTCTACGGTATGCCCCGAGCAG |
| glutamate | | CGGTCGAGCTTAAAGGTGTCATGCATGTGCTGAAGCAGCAAGAA |
| methylesterase/ | | ATTGCAGGCAAATTAATATCCGCAATCGGTTTATCGGCCTTATCTT |
| glutaminase | | AG |

### Example 8: Phosphate Solubilization Genome Analysis

Using the genomic sequence obtained for CK1 and CK2 in Example 6, the genomes of these strains were analyzed for the presence and abundance of phosphate solubilization genes.

To obtain gene candidates for phosphate solubilization in the CK1, CK2 and CK3 strains, a combination of approaches was used to identify a relevant gene set (e.g., related to inorganic phosphate solubilization, phosphate solubilization organic, transport, regulatory genes, and production of organic acids). In those genes in which there was the possibility of building a hidden Markov model from Eggnog's database a similar approach to the identification of nitrogen fixation genes was used from Example 7. However, in most genes it was necessary to manually search for each representative sequence for each strain since the names and annotations vary greatly according to the database and according to the bacterial genus in which the sequences are to be searched.

For the manual search, representative sequences of Klebsiella, and Bacillus were searched in the Uniprot database, preferably choosing the sequences that were manually curated. Then, the crb-blast, prokka, bowtie2, samtools, gffread and cuffinks programs were applied. Each gene sequence was searched in the files previously obtained in the genome annotation. Next, with these results, the ffn file obtained from prokka was searched to create individual files with the nucleotide sequence of each gene found and prokka was used to obtain the gtf files of each gene, which was then used as input. Then, the Bowtie2 program, samtools and gffread programs were used to map each sequence found against the fastq files directly from the sequencer in order to quantify the abundance of each gene. Finally, the program cuffinks was used to calculate the fragments per kilobase per million (FPKM), and thus provide an estimated abundance of each gene. This comparison is optimal for making comparisons with other genes of the same genome but cannot be used for estimates between different genomes. This pipeline was used individually for each gene and for each strain.

Results of the genome analysis are summarized in Table 29. Relative abundance of the phosphate solubilization genes in CK1 and CK2 is shown in FIGS. 8 and 9. Table 30 lists the gene names, enzymes, and protein sequences searched.

The presence of 39 genes related to phosphorus solubilization activity was studied in extremophile genomes. According to their function, the genes were divided into 5 different groups which comprise inorganic phosphorus solubilization, organic phosphorus mineralization, membrane transporters, regulatory genes, and synthesis of organic acids.

CK1 genome exhibited the presence of 27 genes linked to phosphorus solubilization and 17 genes were found in CK2 genome. CK1 showed distinct genes encoding for phosphatases, phytases and organic acids demonstrating its ability to solubilize organic and inorganic phosphate. CK2 had also allocated genes with different functions, however, the diversity of genes was lower than CK1. In addition, gene abundance or the copy number variation (CNV), was measured. The results showed that CK1 not only exhibited a greater gene diversity compared to CK2, but also a higher abundance.

It was concluded that CK1 has phosphate solubilization activity and is a better phosphate solubilizer bacterium than CK2.

TABLE 29
Presence (+) and absence (−) of phosphate solubilization genes

| Gene | Klebsiella aerogenes CK1 | Bacillus cereus CK2 |
|---|---|---|
| pqqA | + | − |
| pqqB | + | − |
| pqqC | + | − |
| pqqD | + | − |
| gcd | + | − |
| gdh | − | + |
| ppa | − | − |
| ppx | − | + |
| aphA | + | − |
| appA | + | − |
| phnF | + | − |
| phnG | + | − |
| phnH | + | − |
| phnI | + | − |
| phnJ | + | − |
| phnK | + | − |
| phnL | + | − |
| phnM | + | − |
| phnN | + | − |
| phoA | + | ++ |
| pgpB/phoC | + | + |
| phoD | − | − |
| phoN | − | − |
| phyC | − | − |
| phnC | − | − |
| phnD | − | − |
| phnE | − | − |
| pstS | − | ++ |
| ugpB | + | + |
| phoP | − | +++ |
| phoR | + | + |
| gspF | + | − |
| gspE | + | − |
| ppc | + | − |
| pyc | − | + |
| gltA | + | + |
| sucD | + | + |
| gad | + | − |
| mdh | + | + |

TABLE 30
Phosphorus solubilization genes

| Gene Name | FASTA protein sequence |
|---|---|
| aphA | >sp\|B5XXX7\|APHA_KLEP3 Class B acid phosphatase OS = Klebsiella |
| | pneumoniae (strain 342) OX = 507522 GN = aphA PE = 3 SV = 1 |
| | MRKLTLAFAAASLLFTLNSAVVARASTPQPLWVGTNVAQLAEQAPIHW |
| | VSVAQIENSLLGRPPMAVGFDIDDTVLFSSPGFWRGQKTFSPGSEDYLKN |
| | PQFWEKMNNGWDEFSMPKEVARQLIAMHVKRGDSIWFVTGRSQTKTET |
| | VSKTLQDDFLIPAANMNPVIFAGDKPGQNTKTQWLQAKQIKVFYGDSD |
| | NDITAAREAGARGIRVLRAANSSYKPLPMAGALGEEVIVNSEY |
| appA | >sp\|P07102\|PPA_ECOLI Periplasmic AppA protein OS = Escherichia coli (strain |
| | K12) OX = 83333 GN = appA PE = 1 SV = 2 |
| | MKAILIPFLSLLIPLTPQSAFAQSEPELKLESVVIVSRHGVRAPTKATQLM |
| | QDVTPDAWPTWPVKLGWLTPRGGELIAYLGHYQRQRLVADGLLAKKG |
| | CPQSGQVAIIADVDERTRKTGEAFAAGLAPDCAITVHTQADTSSPDPLFN |
| | PLKTGVCQLDNANVTDAISRAGGSIADFTGHRQTAFRELERVLNFPQSNL |
| | CLKREKQDESCSLTQALPSELKVSADNVSLTGAVSLASMLTEIFLLQQAQ |
| | GMPEPGWGRITDSHQWNTLLSLHNAQFYLLQRTPEVARSRATPLLDLIK |
| | TALTPHPPQKQAYGVTLPTSVLFIAGHDTNLANLGGALELNWTLPGQPD |
| | NTPPGGELVFERWRRLSDNSQWIQVSLVFQTLQQMRDKTPLSLNTPPGE |
| | VKLTLAGCEERNAQGMCSLAGFTQIVNEARIPACSL |
| gadA | >tr\|COLE03\|COLE03_PSEFL Putative gluconate dehydrogenase |
| | OS = Pseudomonas fluorescens OX = 294 GN = gad PE = 4 SV = 1 |
| | MATVMKKVDAVIVGFGWTGAIMAKELTEAGLNVLALERGPMQDTYPD |
| | GNYPQVIDELTYSVRKKLFQDISKETVTIRHSVNDVALPNRQLGAFLPGN |
| | GVGGAGLHWSGVHFRVDPIELRMRSHYEERYGKNFIPKDMTIQDFGVSY |
| | EELEPFFDYAEKVFGTSGQAWTVKGQLVGDGKGGNPYAPDRSDHFPLE |
| | SQKNTYSAQLFQKAANEVGYKPYNLPSANTSGPYTNPYGAQMGPCNFC |
| | GFCSGYVCYMYSKASPNVNILPALKPLPNFELRPNSHVLRVNLDSSKTRA |
| | TGVTYVDGQGREIEQPADLVILGAFQFHNVRLMLLSGIGKPYDPITGEGV |
| | VGKNFAYQNMATIKAYFDKDVHTNNFIGAGGNGVAVDDFNADNFDHG |
| | PHGFVGGSPMWVNQAGSRPIAGTSNPPGTPAWGSAWKKATADYYTHQ |
| | VSMDAHGAHQSYRGNYLDLDPVYRDAYGLPLLRMTFDWQENDIKMNR |
| | FMVEKMGKIAEAMNPKAIALLGKKVGEHENTASYQTTHLNGGAIMGTD |
| | PKTSALNRYLQSWDVHNVFVPGASAFPQGLGYNPTGLVAALTYWSARA |
| | IREQYLKNPGPLVQA |
| gcd | >sp\|P15877\|DHG_ECOLI Quinoprotein glucose dehydrogenase OS = Escherichia |
| | coli (strain K12) OX = 83333 GN = gcd PE = 1 SV = 3 |
| | MAINNTGSRRLLVTLTALFAALCGLYLLIGGGWLVAIGGSWYYPIAGLV |
| | MLGVAWMLWRSKRAALWLYAALLLGTMIWGVWEVGFDFWALTPRSD |
| | ILVFFGIWLILPFVWRRLVIPASGAVAALVVALLISGGILTWAGFNDPQEI |
| | NGTLSADATPAEAISPVADQDWPAYGRNQEGQRFSPLKQINADNVHNL |
| | KEAWVFRTGDVKQPNDPGEITNEVTPIKVGDTLYLCTAHQRLFALDAAS |
| | GKEKWHYDPELKTNESFQHVTCRGVSYHEAKAETASPEVMADCPRRIIL |
| | PVNDGRLIAINAENGKLCETFANKGVLNLQSNMPDTKPGLYEPTSPPIITD |
| | KTIVMAGSVTDNFSTRETSGVIRGFDVNTGELLWAFDPGAKDPNAIPSDE |
| | HTFTFNSPNSWAPAAYDAKLDLVYLPMGVTTPDIWGGNRTPEQERYASS |
| | ILALNATTGKLAWSYQTVHHDLWDMDLPAQPTLADITVNGQKVPVIYA |
| | PAKTGNIFVLDRRNGELVVPAPEKPVPQGAAKGDYVTPTQPFSELSFRPT |
| | KDLSGADMWGATMFDQLVCRVMFHQMRYEGIFTPPSEQGTLVFPGNLG |
| | MFEWGGISVDPNREVAIANPMALPFVSKLIPRGPGNPMEQPKDAKGTGT |
| | ESGIQPQYGVPYGVTLNPFLSPFGLPCKQPAWGYISALDLKTNEVVWKK |
| | RIGTPQDSMPFPMPVPVPFNMGMPMLGGPISTAGNVLFIAATADNYLRA |
| | YNMSNGEKLWQGRLPAGGQATPMTYEVNGKQYVVISAGGHGSFGTKM |
| | GDYIVAYALPDDVK |
| gdhA | >sp\|P10528\|DHGA_BACME Glucose 1-dehydrogenase A OS = Bacillus |
| | megaterium OX = 1404 GN = gdhA PE = 3 SV = 1 |
| | MYTDLKDKVVVITGGSTGLGRAMAVRFGQEEAKVVINYYNNEEEALDA |
| | KKEVEEAGGQAIIVQGDVTKEEDVVNLVQTAIKEFGTLDVMINNAGVEN |
| | PVPSHELSLDNWNKVIDTNLTGAFLGSREAIKYFVENDIKGNVINMSSVH |
| | EMIPWPLFVHYAASKGGMKLMTETLALEYAPKGIRVNNIGPGAMNTPIN |
| | AEKFADPEQRADVESMIPMGYIGKPEEVAAVAAFLASSQASYVTGITLFA |
| | DGGMTKYPSFQAGRG |
| gltA | >tr\|A0A1Y0XB52\|A0A1Y0XB52_BACAM Citrate synthase OS = Bacillus |
| | amyloliquefaciens OX = 1390 GN = gltA PE = 3 SV = 1 |
| | MTATRGLEGVVATTSSVSSIIDDTLTYVGYDIDDLTENASFEEIIYLLWHL |
| | RLPNKTELAELKKQLAKEAAVPQEIIEHFKSYPLNNVHPMAALRTAISLL |
| | GLTDSEADVMNPEANYRKAIRLQAKVPGIVAAFSRIRKGLDPVEPKEEY |
| | GIAENFLYTLNGEEPSPIEVEAFNKALILHADHELNASTFTARVCVATLSD |
| | IYSGITAAIGALKGPLHGGANEAVMKMLTEIGEVENAEPYIRSKMEKKE |
| | KVMGFGHRVYKHGDPRAKHLKEMSKRLTNLTGESKWYDMSIRVEEIVT |
| | SEKKLPPNVDFYSASVYHSLGIDHDLFTPIFAVSRMSGWIAHILEQYDNN |
| | RLIRPRAEYTGPDKQTFVPIDERA |
| mdh | >tr\|D8GYA5\|D8GYA5_BACAI Malate dehydrogenase OS = Bacillus cereus var. |
| | anthracis (strain CI) OX = 637380 GN = mdh1 PE = 3 SV = 1 |
| | MTIKRKKVSVIGAGFTGATTAFLLAQKELADVVLVDIPQLENPTKGKAL |
| | DMLEASPVQGFDANIIGTSDYADTADSDVVVITAGIARKPGMSRDDLVA |
| | TNSKIMKSITRDIAKHSPNAIIVVLTNPVDAMTYSVFKEAGFPKERVIGQS |
| | GVLDTARFRTFIAQELNLSVKDITGFVLGGHGDDMVPLVRYSYAGGIPLE |
| | TLIPKERLEAIVERTRKGGGEIVGLLGNGSAYYAPAASLVEMTEAILKDQ |
| | RRVLPAIAYLEGEYGYSDLYLGVPVILGGNGIEKIIELELLADEKEALDRS |
| | VESVRNVMKVLV |
| | >tr\|C3SRV3\|C3SRV3_ECOLX Malate dehydrogenase OS = Escherichia coli |
| | OX = 562 GN = mdh PE = 3 SV = 1 |
| | MKVAVLGAAGGIGQALALLLKTQLPSGSELSLYDIAPVTPGVAVDLSHIP |
| | TAVKIKGFSGEDATPALEGADVVLISAGVARKPGMDRSDLFNVNAGIVK |
| | NLVQQVAKTCPKACIGIITNPVNTTVAIAAEVLKKAGVYDKNKLFGVTT |
| | LDIIRSNTFVAELKGKQPGEVEVPVIGGHSGVTILPLLSQVPGVSFTEQEV |
| | ADLTKRIQNAGTEVVEAKAGGGSATLSMGQAAARFGLSLVRALQGEQG |
| | VVECAYVEGDGQYARFFSQPLLLGKNGVEERKSIGTLSAFEQNALEGML |
| | DTLKKDIALGEEFVNK |
| phnC | >sp\|Q81A96\|PHNC_BACCR Phosphonates import ATP-binding protein PhnC |
| | OS = Bacillus cereus (strain ATCC 14579/DSM 31/CCUG 7414/JCM 2152/ |
| | NBRC 15305/NCIMB 9373/NCTC 2599/NRRL B-3711) OX = 226900 |
| | GN = phnC PE = 3 SV = 2 |
| | MIEFRNVSKVYPNGTKGLNNINLKIQKGEFVIMVGLSGAGKSTLLKSVN |
| | RLHEITEGEIMIECESITAAKGKDLRRMRRDIGMIFQSFNLVKRSTVLKNV |
| | LAGRVGYHSTLRTTLGLFPKEDVELAFQALKRVNILEKAYARADELSGG |
| | QQQRVSIARALAQEAKIILADEPVASLDPLTTKQVLDDLKKINEDFGITTI |
| | VNLHSIALARQYATRIIGLHAGEIVFDGLVEAATDEKFAEIYGDVAQKSE |
| | LLEVAAK |
| phnD | >tr\|A0A0C7KIY9\|A0A0C7KIY9_KLEPN Phosphonate ABC transporter ATP- |
| | binding protein OS = Klebsiella pneumoniae OX = 573 GN = phnC PE = 4 SV = 1 |
| | MNSSLAAVAETDFQPFTDPAAGRQRKVLSVRNLSKAYQAQHKVLDGISF |
| | DLHAGEMVGVIGRSGAGKSTLLHVLNGTHSASGGEILSYPEVGTPHDVS |
| | QLKGRALNAWRSHCGMIFQDFCLVPRLDVLTNVLLGRLSQTSTLKSLFK |
| | IFPAADRARAIALLEWMNMLPHALQRAENLSGGQMQRVAICRALMQNP |
| | GILLADEPVASLDPKNTQRIMDVLREISEQGISVMVNLHSVELVRAYCTR |
| | VIGVASGQLIFDDHPSRLTQDVLQRLYGDEVSQLH |
| | >tr\|A0A6M0Q113\|A0A6M0Q113_9BACI Phosphate/phosphite/phosphonate |
| | ABC transporter substrate-binding protein OS = Bacillus mesophilus |
| | OX = 1808955 GN = phnD PE = 3 SV = 1 |
| | MKKLWVMMIIALFAVLAACGGGNDEVKEEEQVENTEEQTASEELEKLV |
| | VGVIPSLNQGNMQTAMDKLSKHFESELDIPVEITVYPDYQAVVQAMNY |
| | DEVNMAYFGPSTYIDANEQSGARAIMTQLIDGEPFYYSYIITHKDSPLTSI |
| | EDLVAQSKDLTFAFGDPSSTSGSLIPSIELKKQGVFTNQNEHQFDNLLYT |
| | GGHDATALAVENKQVDAGAIDSAIFDTLQANGKIGDNFKIIWQSEKLFQ |
| | YPWAVSKVVSDELVAKIQDAFLNVKDQEILDAFAATGFTVATDADYEAI |
| | REAKKEAK |
| phnE | >tr\|A0A086IT87\|A0A086IT87_KLEPN Phosphate-import protein PhnD |
| | OS = Klebsiella pneumoniae OX = 573 GN = phnD PE = 3 SV = 1 |
| | MKKYMTGAVRLSAMVAGMMMAWQAAAAQPKELNLGILGGQNATQQI |
| | GDNQCVKAFLDKELNVDTKLRNSSDYSGVIQGLLGGKVDVVLSMSPSS |
| | YASVYLNNPKAVDIVGIAVDDKDQSRGYHSVVIVKADSPYKTLDDLKG |
| | KAFGFADPDSTSGYLIPNHAFKEKFGGNADNKYNNTFSSVTFSGGHEQDI |
| | LGVLNGQFAGAVTWASMVGDYNTGYTTGAFNRLIRMDHPDLMKQIRII |
| | WQSPLIPNGPILVSNALPADFKAKVVAAVKKLDTEDHACFIKAMGGTQH |
| | IGPGSVADFQQIIDMKRELVSAR |
| | >tr\|W9BNE6\|W9BNE6_KLEPN Phosphate-import permease protein PhnE |
| | OS = Klebsiella pneumoniae OX = 573 GN = phnE_2 PE = 3 SV = 1 |
| | MKTTHTEFERYYQQVRSRQKRDAVCWSLLLLALYFAAGSAAEFNLLTI |
| | WHSLPHFFDYMAETIPPLSAGNLFADVQTKGSLAWWGYRLPIQLPLIWE |
| | TLQLALASTLVAVAIATVFAFLAANNAWSPAPVRFAIRVLVAFLRTMPE |
| | LAWAVIFVMAFGIGAIPGFLALMLHTVGSLTKLFYEAVESAQNKPVRGL |
| | AACGASPLQKIRFALWPQVKPLFLSYGFMRLEINFRSSTILGLVGAGGIG |
| | QELMTNIKLDRYDQVSITLLLIILVVSALDMLSGRLRLWVLEGKK |
| phnG | >tr\|A0A0G8C4K6\|A0A0G8C4K6_BACCE Phosphate-import permease protein |
| | PhnE OS = Bacillus cereus OX = 1396 GN = phnE PE = 3 SV = 1 |
| | MNDVTIYSKSIPKPPSKLKHMLTAVLVILLLWGSSVQVDASLSKLVVGFP |
| | NMMDLLKEMVPPDWSYFQVITTAMLDTIRMAIIGTTLGAILAIPLALFAA |
| | SNVFTSTFLYSPARMILNFIRTIPDLLLAAIFVAIFGIGPLPGILALTFFSIGL |
| | VAKLLYESIESIDPGPLEAMTAVGANKVKWIVYGVIPQVKAHFVSYVLY |
| | TFEVNVRAAAVLGLVGAGGIGLYYDRTLGFLQYQQTASIIIYTLVVVLLI |
| | DYVSTLLREKL |
| phnJ | >tr\|A0A2G0YHS1\|A0A2G0YHS1_9PSED Phosphonate C-P lyase system |
| | protein PhnG OS = Pseudomonas sp. ICMP 8385 OX = 1718920 |
| | GN = A0268 29955 PE = 4 SV = 1 |
| | MNLSPRQHWIGVLARAQLNELQPHEAALKDAEYQLIRAPEIGMTLVRGR |
| | MGGNGAPFNVGEMTVTRCVVRLADGRTGYSYLAGRDKVHAELAALAD |
| | AHLQNTPPSPWLTDLISALAQAQARRRAQKEADTAATKVEFFTLVRGEN |
| | G |
| phoA | >tr\|Q9XB36\|Q9XB36_KLEAE C-P lyase subunit (Fragment) OS = Klebsiella |
| | aerogenes OX = 548 GN = phnJ PE = 4 SV = 1 |
| | ADDTTNAVSIRQFFKRVTGVATTERTEDATLIQTRHRIPETPLTDDQILIFQ |
| | VPIPEPLRFIEPRETETRTMHALEEYGVMQVKLYEDIARFGHIATTYAYPV |
| | KVNGRYVMDPSPIPKFDNPKMHMMPALQLFGAG |
| | >sp\|P00634\|PPB_ECOLI Alkaline phosphatase OS = Escherichia coli (strain |
| | K12) OX-83333 GN = phoA PE = 1 SV = 1 |
| | MKQSTIALALLPLLFTPVTKARTPEMPVLENRAAQGDITAPGGARRLTG |
| | DQTAALRDSLSDKPAKNIILLIGDGMGDSEITAARNYAEGAGGFFKGIDA |
| | LPLTGQYTHYALNKKTGKPDYVTDSAASATAWSTGVKTYNGALGVDIH |
| | EKDHPTILEMAKAAGLATGNVSTAELQDATPAALVAHVTSRKCYGPSA |
| | TSEKCPGNALEKGGKGSITEQLLNARADVTLGGGAKTFAETATAGEWQ |
| | GKTLREQAQARGYQLVSDAASLNSVTEANQQKPLLGLFADGNMPVRW |
| | LGPKATYHGNIDKPAVTCTPNPQRNDSVPTLAQMTDKAIELLSKNEKGF |
| | FLQVEGASIDKQDHAANPCGQIGETVDLDEAVQRALEFAKKEGNTLVIV |
| | TADHAHASQIVAPDTKAPGLTQALNTKDGAVMVMSYGNSEEDSQEHTG |
| | SQLRIAAYGPHAANVVGLTDQTDLFYTMKAALGLK |
| phoB | >sp\|P19406\|PPB4_BACSU Alkaline phosphatase 4 OS = Bacillus subtilis (strain |
| | 168) OX = 224308 GN = phoA PE = 1 SV = 4 |
| | MKKMSLFQNMKSKLLPIAAVSVLTAGIFAGAELQQTEKASAKKQDKAEI |
| | RNVIVMIGDGMGTPYIRAYRSMKNNGDTPNNPKLTEFDRNLTGMMMTH |
| | PDDPDYNITDSAAAGTALATGVKTYNNAIGVDKNGKKVKSVLEEAKQQ |
| | GKSTGLVATSEINHATPAAYGAHNESRKNMDQIANSYMDDKIKGKHKI |
| | DVLLGGGKSYFNRKDRNLTKEFKQAGYSYVTTKQALKKNKDQQVLGL |
| | FADGGLAKALDRDSKTPSLKDMTVSAIDRLNQNKKGFFLMVEGSQIDW |
| | AAHDNDTVGAMSEVKDFEQAYKAAIEFAKKDKHTLVIATADHTTGGFT |
| | IGANGEKNWHAEPILSAKKTPEFMAKKISEGKPVKDVLARYANLKVTSE |
| | EIKSVEAAAQADKSKGASKAIIKIFNTRSNSGWTSTDHTGEEVPVYAYGP |
| | GKEKFRGLINNTDQANIIFKILKTGK |
| phoD | >sp\|P0AFJ5\|PHOB_ECOLI Phosphate regulon transcriptional regulatory protein |
| | PhoB OS = Escherichia coli (strain K12) OX = 83333 GN = phoB PE = 1 SV = 1 |
| | MARRILVVEDEAPIREMVCFVLEQNGFQPVEAEDYDSAVNQLNEPWPDL |
| | ILLDWMLPGGSGIQFIKHLKRESMTRDIPVVMLTARGEEEDRVRGLETG |
| | ADDYITKPFSPKELVARIKAVMRRISPMAVEEVIEMQGLSLDPTSHRVMA |
| | GEEPLEMGPTEFKLLHFFMTHPERVYSREQLLNHVWGTNVYVEDRTVD |
| | VHIRRLRKALEPGGHDRMVQTVRGTGYRFSTRF |
| | >sp\|Q5NNZ8\|ALPH_ZYMMO Alkaline phosphatase PhoD OS = Zymomonas |
| | mobilis subsp. mobilis (strain ATCC 31821/ZM4/CP4) OX = 264203 |
| | GN = phoD PE = 1 SV = 1 |
| | MNSLLHHSFLKTVFSSLAIAIVTSSLSSVTIAATHPLDNHPKGEIAASSETA |
| | HNPWSGTRLIVAISVDQFSSDLFSEYRGRFRSGMKQLQNGVVYPMAYHS |
| | HAATETCPGHSVLLTGDHPARTGIIANNWYDFSVKRADKKVYCSEDPSL |
| | SADPQNYQPSVHYLKVPTLGDRMKKANPHSRVISVAGKDRAAIMMGGH |
| | MTDQIWFWSDNAYKTLADHKGEMPVTVKTVNEQVTRFMQQDEAPVM |
| | PSVCADHASALKIGNNRIIGLAPASRKAGDFKTFRVTPDYDRTTTDIAIGL |
| | IDELKLGHGNAPDLLTVSLSATDAVGHAYGTEGAEMCSQMAGLDDNIA |
| | RIIAALDSNGVPYVLVLTADHGGQDVPERAKLRGVETAQRVDPALSPDQ |
| | LSLRLAERFQLSHNQPLFFANEPQGDWYINRNLPEQTKAQLIQAAKSELS |
| | NHPQVAAVFTASELTHIPYPTRSPELWNLAERAKASFDPLRSGDLIVLLK |
| | PRVTPIAKPVSYVATHGSAWDYDRRVPIIFYTPHASGFEQPMPVETVDIM |
| | PSLAALLQIPLRKGEVDGRCLDLDPTEATTCPVK |
| phoN | >sp\|P42251\|PPBD_BACSU Alkaline phosphatase D OS = Bacillus subtilis (strain |
| | 168) OX = 224308 GN = phoD PE = 1 SV = 3 |
| | MAYDSRFDEWVQKLKEESFQNNTFDRRKFIQGAGKIAGLSLGLTIAQSV |
| | GAFEVNAAPNFSSYPFTLGVASGDPLSDSVVLWTRLAPDPLNGGGMPKQ |
| | AVPVKWEVAKDEHFRKIVRKGTEMAKPSLAHSVHVEADGLEPNKVYY |
| | YRFKTGHELSPVGKTKTLPAPGANVPQMTFAFASCQQYEHGYYTAYKH |
| | MAKEKLDLVFHLGDYIYEYGPNEYVSKTGNVRTHNSAEIITLQDYRNRH |
| | AQYRSDANLKAAHAAFPWVVTWDDHEVENNYANKIPEKGQSVEAFVL |
| | RRAAAYQAYYEHMPLRISSLPNGPDMQLYRHFTYGNLASFNVLDTRQY |
| | RDDQANNDGNKPPSDESRNPNRTLLGKEQEQWLFNNLGSSTAHWNVLA |
| | QQIFFAKWNFGTSASPIYSMDSWDGYPAQRERVINFIKSKNLNNVVVLT |
| | GDVHASWASNLHVDFEKTSSKIFGAEFVGTSITSGGNGADKRADTDQIL |
| | KENPHIQFFNDYRGYVRCTVTPHQWKADYRVMPFVTEPGAAISTRASFV |
| | YQKDQTGLRKVSSTTIQGGVKQSDEVEEDRFFSHNKAHEKQMIKKRAKI |
| | TN |
| phoR | >sp\|P26976\|PHON_SALTY Non-specific acid phosphatase OS = Salmonella |
| | typhimurium (strain LT2/SGSC1412/ATCC 700720) OX-99287 GN = phoN |
| | PE = 3 SV = 1 |
| | MKSRYLVFFLPLIVAKYTSAETVQPFHSPEESVNSQFYLPPPPGNDDPAY |
| | RYDKEAYFKGYAIKGSPRWKQAAEDADVSVENIARIFSPVVGAKINPKD |
| | TPETWNMLKNLLTMGGYYATASAKKYYMRTRPFVLFNHSTCRPEDENT |
| | LRKNGSYPSGHTAYGTLLALVLSEARPERAQELARRGWEFGQSRVICGA |
| | HWQSDVDAGRYVGAVEFARLQTIPAFQKSLAKVREELNDKNNLLSKED |
| | HPKLNY |
| | >sp\|P08400\|PHOR_ECOLI Phosphate regulon sensor protein PhoR |
| | OS = Escherichia coli (strain K12) OX = 83333 GN = phoR PE = 1 SV = 1 |
| | MLERLSWKRLVLELLLCCLPAFILGAFFGYLPWFLLASVTGLLIWHFWN |
| | LLRLSWWLWVDRSMTPPPGRGSWEPLLYGLHQMQLRNKKRRRELGNLI |
| | KRFRSGAESLPDAVVLTTEEGGIFWCNGLAQQILGLRWPEDNGQNILNL |
| | LRYPEFTQYLKTRDFSRPLNLVLNTGRHLEIRVMPYTHKQLLMVARDVT |
| | QMHQLEGARRNFFANVSHELRTPLTVLQGYLEMMNEQPLEGAVREKAL |
| | HTMREQTQRMEGLVKQLLTLSKIEAAPTHLLNEKVDVPMMLRVVEREA |
| | QTLSQKKQTFTFEIDNGLKVSGNEDQLRSAISNLVYNAVNHTPEGTHITV |
| | RWQRVPHGAEFSVEDNGPGIAPEHIPRLTERFYRVDKARSRQTGGSGLG |
| | LAIVKHAVNHHESRLNIESTVGKGTRFSFVIPERLIAKNSD |
| phoP | >sp\|P23545\|PHOR_BACSU Alkaline phosphatase synthesis sensor protein PhoR |
| | OS = Bacillus subtilis (strain 168) OX = 224308 GN = phoR PE = 1 SV = 1 |
| | MNKYRVRLFSVFVVCMILVFCVLGLFLQQLFETSDQRKAEEHIEKEAKY |
| | LASLLDAGNLNNQANEKIIKDAGGALDVSASVIDTDGKVLYGSNGRSAD |
| | SQKVQALVSGHEGILSTTDNKLYYGLSLRSEGEKTGYVLLSASEKSDGL |
| | KGELWGMLTASLCTAFIVIVYFYSSMTSRYKRSIESATNVATELSKGNYD |
| | ARTYGGYIRRSDKLGHAMNSLAIDLMEMTRTQEMQRDRLLTVIENIGSG |
| | LIMIDGRGFINLVNRSYAKQFHINPNHMLRRLYHDAFEHEEVIQLVEDIF |
| | MTETKKCKLLRLPIKIERRYFEVDGVPIMGPDDEWKGIVLVFHDMTETK |
| | KLEQMRKDFVANVSHELKTPITSIKGFTETLLDGAMEDKEALSEFLSIILK |
| | ESERLQSLVQDLLDLSKIEQQNFTLSIETFEPAKMLGEIETLLKHKADEKG |
| | ISLHLNVPKDPQYVSGDPYRLKQVFLNLVNNALTYTPEGGSVAINVKPR |
| | EKDIQIEVADSGIGIQKEEIPRIFERFYRVDKDRSRNSGGTGLG |
| | LAIVKHLIEAHEGKIDVTSELGRGTVFTVTLKRAAEKSA |
| phyC | >sp\|P13792\|PHOP_BACSU Alkaline phosphatase synthesis transcriptional |
| | regulatory protein PhoP OS = Bacillus subtilis (strain 168) OX = 224308 |
| | GN = phoP PE = 1 SV = 4 |
| | MNKKILVVDDEESIVTLLQYNLERSGYDVITASDGEEALKKAETEKPDLI |
| | VLDVMLPKLDGIEVCKQLRQQKLMFPILMLTAKDEEFDKVLGLELGAD |
| | DYMTKPFSPREVNARVKAILRRSEIAAPSSEMKNDEMEGQIVIGDLKILP |
| | DHYEAYFKESQLELTPKEFELLLYLGRHKGRVLTRDLLLSAVWNYDFAG |
| | DTRIVDVHISHLRDKIENNTKKPIYIKTIRGLGYKLEEPKMNE |
| | >tr\|A0A119A2M7\|A0A119A2M7_9PSED 3-phytase OS = Pseudomonas sp. |
| | TAD18 OX = 1729583 GN = phyC PE = 4 SV = 1 |
| | MRFNCKPCLLPLLISLSAGHAQAATPVTAPTLKPWSATKAQALGWLAG |
| | DQRLAVSKREGVLLLDAQGKTLSHVPGAFASLDSRALGDQVLVASHDE |
| | KKQQVALFSLNPQSHEWLAPVYLPRRDYAVNGVCLYRDEASNIYLFTV |
| | GEEGKGEQWLVAADRKRLNQPRLVRSLPLPPEAGLCQVDDAAHQLFVN |
| | EQKVGWWAYPAHAEAQASRVPVAMIEPFGEVKQAAGAMVPVPGGML |
| | GLDPKAGELHLYQQQGKGWSPVARFPLKPLVEPEHLAVRQTPQGLDVW |
| | VQDADNNQLFEGRLSWNPVPVSVPPVLPVVKPSVQTDPVVSQGDAADD |
| | PAIWLHPHDLALSRVLGTNKKNGLEVYDLQGRRVQHLEVGRLNNVDVR |
| | PDFKLGTRTVDLAVATNRDHNSLSVFSIDRATGEVRAAGEVPTPLKDIYG |
| | LCLFKAPTGEIYSFANDKDGTFLQHRLSAKGEQVQGELVRQFKVATQPE |
| | GCVADDTHQRLFIGEEDVAVWALDARPEQPAALSSVINVGGPVHDDIEG |
| | LALYQGEKNSYLVISSQGNDSYVVLDAQPPYALRGAFRVGVNAEAGIDG |
| | ASETDGLEVTSANLGGPFTQGMLVVQDGRKRMPEHSQNYKYIPWADVA |
| | KTLNLP |
| ppa | >sp\|O31097\|PHYC_BACIU 3-phytase OS = Bacillus subtilis OX = 1423 |
| | GN = phyC PE = 1 SV = 1 |
| | MNHSKTLLLTAAAGLMLTCGAVSSQAKHKLSDPYHFTVNAAAETEPVD |
| | TAGDAADDPAIWLDPKTPQNSKLITTNKKSGLVVYSLDGKMLHSYNTG |
| | KLNNVDIRYDFPLNGKKVDIAAASNRSEGKNTIEIYAIDGKNGTLQSMTD |
| | PDHPIATAINEVYGFTLYHSQKTGKYYAMVTGKEGEFEQYELKADKNG |
| | YISGKKVRAFKMNSQTEGMAADDEYGRLYIAEEDEAIWKFSAEPDGGS |
| | NGTVIDRADGRHLTRDIEGLTIYYAADGKGYLMASSQGNSSYAIYDRQG |
| | KNKYVADFRITDGPETDGTSDTDGIDVLGFGLGPEYPFGIFVAQDGENID |
| | HGQKANQNFKIVPWERIADQIGFRPLANEQVDPRKLTDRSGK |
| | >tr\|A0A0H3GY84\|A0A0H3GY84_KLEPH Inorganic pyrophosphatase |
| | OS = Klebsiella pneumoniae subsp. pneumoniae (strain HS11286) OX = 1125630 |
| | GN = ppa PE = 3 SV = 1 |
| | MHLVKTILTAGLLLSAAAQAHNVLEFPQPENNPEEFYAVTEIPTGGIIKYE |
| | TDAKTGFIVADRFQSMPVAYPANYGSLTQSLAGDGDPLDVVFYTRAPM |
| | APGTLIKLRAIGVLKMIDGGEKDDKIIAVPASKIDPTYDDIKTISDLPKIEQ |
| | QRLEAFFRVYKELPEGRKRSSWPALMTPRPRSRRSNRPGRPGRRRTRNNI |
| | HRGRCPAPAAFAFTIPMAAKSHVTRHTTTLIPPYCILTQDEGDGCIVQPFH |
| | FLDPPPCLIPP |
| ppc | >sp\|P19514\|IPYR_BACP3 Inorganic pyrophosphatase OS = Bacillus sp. (strain |
| | PS3) OX = 2334 GN = ppa PE = 1 SV = 2 |
| | MAFENKIVEAFIEIPTGSQNKYEFDKERGIFKLDRVLYSPMFYPAEYGYL |
| | QNTLALDGDPLDILVITTNPPFPGCVIDTRVIGYLNMVDSGEEDAKLIGVP |
| | VEDPRFDEVRSIEDLPQHKLKEIAHFFERYKDLQGKRTEIGTWEGPEAAA |
| | KLIDECIARYNEQK |
| ppx | >tr\|A0A328LFZ7\|A0A328LFZ7_9BACI Phosphoenolpyruvate carboxylase |
| | OS = Bacillus sp. SRB_336 OX = 1969380 GN = ppc PE = 3 SV = 1 |
| | MSSELPAITPQNTQDPAGDTDLRRDVRRVSTLLGESLVRQHGPELLAMV |
| | EQVRLLTKESKEAARGETGTGPWSANDVAEQVREVLASLPLEQATDLV |
| | RAFAFYFHLANAAEQVHRVRSLRARAEEDGWLARTVAEIAEKAGAGAL |
| | QKVVNELDVRPIFTAHPTEASRRSVLDKVRKLSDILATPSAEGSSARSRQ |
| | DRQLAEIIDQMWQTDELRKVRPTPMDEARNAIYYLNNILTDAMPEVLTE |
| | LSGLLGAHGVTLPEDAAPLRFGSWIGGDRDGNPNVTADVTRDVLVLQN |
| | ANAVKISVAMVDELLSVLSNSSSLSGADAQLLESITVDLANLPGIAPRVL |
| | ELNAEEPYRLKLTCIKAKLLNTRRRVAADAHHEPGRDYASTAELLADLA |
| | LLETSLRNNSAALVADGALAAVRRAVASFGLHLATLDIREHADHHHDA |
| | VGQLLDRVGELPGPYAELDRAGRLQVLSRELASHRPLSGHPIKLDGAAD |
| | GTYDVFRVVRRALRTYGPDVVETYIISMTRGADDVLAPAVLAREAGLV |
| | QLTGDNRYAKIGFAPLLETVEELRASGEIVDRLLSDPSYRELVRLRGNVQ |
| | EIMLGYSDSNKESGVMTSQWEIHKTQRKLRDVAVKHGVNVRMFHGRG |
| | GSVGRGGGPTYDAILAQPNGVLEGAIK |
| | FTEQGEVISDKYSLPELARENLELSLAAVMQGSALHQAPRHTEDDLVRF |
| | GEVMEIVSGAAFASYRALIDDGDLPAYFLASTPVEQLGSLNIGSRPSKRP |
| | DSGSGLGGLRAIPWVFGWTQSRQIVPGWFGVGSGLKAAREAGREAELA |
| | EMLAHWHFFSSTISNVEMTLAKTDMDIAAHYVRTLVPESLAHLFATIRA |
| | EYDLTVAEIQRLTGEAELLDKQPLLKRSLNVRDQYLDPISYLQVELLRRV |
| | REAGIAKAEVDERLQRAMLITINGVAAGLRNTG |
| | >tr\|W9BC06\|W9BC06_KLEPN Exopolyphosphatase OS = Klebsiella |
| | pneumoniae OX = 573 GN = ppx PE = 3 SV = 1 |
| | MPINDNTPRPQEFAAVDLGSNSFHMVIARVVDGAMQIIGRLKQRVHLAD |
| | GLDENSVLSEEAMTRGLNCLSLFAERLQGFSPSSVCIVGTHTLRQATNAA |
| | EFLKRAEKVIPYPIEIISGNEEARLIFMGVEHTQPERGRKLVIDIGGGSTEL |
| | VIGEDFEPRLVESRRMGCVSFSQAYFPGGVINKENFQRARLAAVQKLETL |
| | AWQFRIQGWTVALGASGTIKAAQEVLVAMGEKDGFITPERLEMLVNEL |
| | LKHKNFDALSLPGLSEDRKAVFAPGLAILCGVFDALAIKELRLSDGALRE |
| | GVLYEMEGRFRHQDIRSRTAQSLANQYNIDREQARRVLETTTQMLEQW |
| | QEQNPKLANPHLAALLKWAVMLHEVGLNINHSGMHRHSAYILQNSDLP |
| | GFNQEQQMLMATLVRYHRKAIKLDDLPRFTLFRKKQFLPLIQLLRLGVL |
| | LNNQRQATTTPPTLRLQTEAHHWTLTFPHNWFSQNALVLLDLEKEQQY |
| | WEGVPEWMLKIAEEEPDA |
| pqq | >tr\|A0A0K6LY49\|A0A0K6LY49_BACCE Exopolyphosphatase OS = Bacillus |
| | cereus OX = 1396 GN = ppx PE = 3 SV = 1 |
| | MKEILKQQYAIIDIGSNTMRLVIYEKQNGGFYKEIENTKVVARLRNYLVD |
| | GVLIEEGIEVLLQTLFQFQESTRFHQLHHVLCVATATIRQAKNQEEIKKLV |
| | EGQTDFTLRVLSEYEEARYGYLAVMNSTSFSEGITVDIGGGSTEVTYFRN |
| | REILEYHSFPFGALSLKQQFIKDDIPTEEELEKLQTYLEYQFRTLPWLIDK |
| | KLPLIAIGGSARNLVKIHQNLICYPIAGVHLYKMKEEDIKNVKEELEALSF |
| | IELQKLEGLAKDRADTIVPAVEVFHTLVNVIEAPAFVLSRKGLREGVFYE |
| | ELTKDLGISYYPNVVEESLYLLSHEYEMDMEFVMQLIKHGRLICQQLEET |
| | GLISMSVEDWEVFHQAAKVFNIGKYIDEEASRLHTFYLLANKTIDGMMH |
| | KERVRLALIASYKSKMLFKQHLSPFEGWFDKNEQKKIRLLGAVLQFSAA |
| | LNIRQRSLVESISITESKEGLTFEIVCEQSALAEKVQAEKQKKQLERVLKT |
| | NIILLFKLKN |
| | >sp\|P27503\|PQQA_KLEPN Coenzyme PQQ synthesis protein A OS = Klebsiella |
| | pneumoniae OX = 573 GN = pqqA PE-3 SV = 1 |
| | MWKKPAFIDLRLGLEVTLYISNR |
| pqqA | >tr\|A0A0D1XIK3\|A0A0D1XIK3_ANEMI Pyrroloquinoline quinone (PQQ) |
| | biosynthesis protein C OS = Aneurinibacillus migulanus OX = 47500 |
| | GN = AF333 24905 PE = 4 SV = 1 |
| | MAITSYEQIEEKIWDIVETEIIQGEFMQTLLAGEWTPAQVREFALQYSYYS |
| | RNFPRVLGAAIAAVEPEDDWWVPLVDNLWDEGGRGNPKSYHSRLYHSF |
| | MITAAPDVPTNEKYVPDYPVSPASKEAVNTFISFLRNATPLEAMASIGFG |
| | SELFAGKVMGLIGQGLEHPNYNRAQKLNTTFWTVHADHHEPRHYELCK |
| | NVLTRFTSSQDLEHMYRAGAYITRSEARFYDGLYERMKSV |
| pstS | >sp\|P27503\|PQQA_KLEPN Coenzyme PQQ synthesis protein A OS = Klebsiella |
| | pneumoniae OX = 573 GN = pqqA PE = 3 SV = 1 |
| | MWKKPAFIDLRLGLEVTLYISNR |
| | >tr\|A0A3S4JIM4\|A0A3S4JIM4_KLEAE Phosphate-binding protein PstS |
| | OS = Klebsiella aerogenes OX = 548 GN = pstS PE-3 SV = 1 |
| | MNVMRTTVATVVAATLSMSAFSAFAAASLTGAGATFPAPVYAKWADT |
| | YQKETGNKVNYQGIGSSGGVKQIIANTVDFGASDAPLADDKLTQEGLFQ |
| | FPTVIGGVVLAVNLPGVKSGELVLDGKTLGDIYLGKIKKWDDEAIAKLN |
| | PGLKLPSQNIAVVRRADGSGTSFVFTSYLSKVNEEWKSKIGAGSTVNWP |
| | TGLGGKGNDGIAAFVQRLPGSIGYVEYAYAKQNNLAYTKLVSADGKPV |
| | SPTEDNFANAAKGVDWSKSFAQDLTNQKGENAWPITSTTFILVHKATNK |
| | PEQTAEVLKFFDWAYKNGGKEANALDYATLPEKRGRAGSRGMENQRQ |
| | RQQR |
| | >sp\|P46338\|PSTS_BACSU Phosphate-binding protein PstS OS = Bacillus subtilis |
| | (strain 168) OX = 224308 GN = pstS PE = 3 SV = 1 |
| | MKKNKLVLMLLMAAFMMIAAACGNAGESKKSNSDSAKGEEKASGSLTI |
| | SGSSAMQPLVLAAAEKFMEENPDADIQVQAGGSGTGLSQVSEGAVQIGN |
| | SDVFAEEKEGIDAKALVDHQVAVVGMAAAVNPDAGVKDISKDELKKIF |
| | TGKIKNWKELGGKDQKITLVNRPDSSGTRATFVKYALDGAEPAEGITED |
| | SSNTVKKIIADTPGAIGYLAFSYLTDDKVTALSIDGVKPEAKNVATGEYPI |
| | WAYQHSYTKGEATGLAKEFLDYLKSEDIQKSIVTDQGYIPVTDMKVTRD |
| | ANGKQS |
| ugpB | >tr\|W9BBW5\|W9BBW5_KLEPN Glycerol-3-phosphate ABC transporter |
| | OS = Klebsiella pneumoniae OX = 573 GN = ugpB PE = 3 SV = 1 |
| | MISLRHTALGLALSLAFAGQALAVTTIPFWHSMEGELGKEVDSLAQREN |
| | AANPDYKIVPVYKGNYEQSLSAGIAAFRTGNAPAILQVYEVGTATMMAS |
| | KAIKPVYQVFSEAGIKFDESQFVPTVAGYYTDSKTGHLLSQPFNSSTPVL |
| | YYNKDAFKKAGLDPDQPPKTWQDLAAYTAKLKAAGMKCGYASGWQG |
| | WIQIENFSAWHGLPVATKNNGFDGTDAVLEFNKPEQVKHIALLEEMNK |
| | KGDFSYFGRKDESTEKFYNGDCAITTASSGSLADIRQYAKFNYGVGMMP |
| | YDADVKGAPQNAIIGGASLWVMQGKDKETYTGVAKFLDFLTKPENAAE |
| | WHQKTGYLPITTAAYDLTRQQGFYDKNPGADIATRQMLNKPPLPFTKGL |
| | RLGNMPQIRTIVDEELESVWTGKKTPQQALDSAVQRGNQLLRRFEQATK |
| | S |
| | >tr\|A0A1BIL0E8\|A0A1BIL0E8_BACTU sn-glycerol-3-phosphate-binding |
| | periplasmic protein UgpB OS = Bacillus thuringiensis OX = 1428 GN = ugpB |
| | PE = 3 SV = 1 |
| | MSLVKKGAALLMAATMALSSAACSNSKTEGKPEALAKVAPVEKNGDK |
| | TVIRFWHAMGGKTQGVLDGLVADYNKSQNKYEIKAEFQGTYEESLTKF |
| | RTMSATKEAPALVQSSEITTKYMIDSKKITPIDSWIKKDKYDTSKLEKAIT |
| | NYYSVDGKMYSMPFNSSTPVLIYNKDAFAKAGLDPEKAPKTYAELQEA |
| | AKKLTIKEGGNVKQYGFSMLNYGWFFEELLATQGALYVDNENGRKDA |
| | AKKAVFNDKEGQKVFGMLDDLNKAGALGKYGASWDDIRAAFQSGQV |
| | AMYLDSSAGVRDLIDASKFNVGVSYIPYPEDSKQNGVVIGGASLWMTN |
| | MVSEETQQGAWDFMKYLTKPDVQAKWHTATGYFSINPDAYNEPLVKE |
| | QYEKYPQLKVTVDQLQATKQSPATQGALISVFPESRDAVVKALEAMYD |
| | GKNSKEALDEAAKATDRAISISARTSQK |

### Example 9: Additional Genomic Characterization of CK1 and CK2

Using the genomic sequence obtained for CK1 and CK2 in Example 6, the genomes of these strains were further analyzed for the presence of virulence genes (Table 31), osmotic stress-related genes (Table 32) and plant growth promoting genes (Tables 33, 35, and 46) using similar methods to Examples 7 and 8.

TABLE 31
Genome analysis of virulence genes

| | Klebsiella aerogenes CK1 | Bacillus cereus CK2 |
|---|---|---|
| Victors | 144 | 2 |
| PATRIC_VF | 117 | 2 |
| VFDB | 42 | 0 |

TABLE 32
Genome analysis of osmotic stress tolerance genes

| | Klebsiella | Bacillus |
|---|---|---|
| | aerogenes | cereus CK2 |
| Strain | CK1 gene count | gene count |
| Osmotic stress cluster | 4 | 0 |
| Choline uptake and conversion to | 12 | 11 |
| betaine clusters | | |
| Universal stress protein family | 7 | 1 |
| Osmoregulation | 4 | 1 |
| EnvZ and OmpR regulon | 4 | 0 |
| Hyperosmotic potassium uptake | 2 | 0 |

TABLE 33
Genome analysis of plant growth promoting genes CK1 and CK2

| | CK1 Klebsiella | CK2 Bacillus |
|---|---|---|
| Function | aerogenes | cereus |
| Phosphate solubilization and | PRESENT | PRESENT |
| mineralization | | |
| Nitrogen assimilation and reduction | PRESENT | PRESENT |
| Nitrogen fixation | MISSING | MISSING |
| Siderophore synthesis/Fe-uptake | PRESENT | PRESENT |
| L-tryptophane, indole synthesis | PRESENT | PRESENT |
| Auxin (Indole-3-Acetic acid) | INCOMPLETE | Detailed |
| synthesis | | analysis |
| | | required |
| ACC (1-Aminocyclopropane-1- | MISSING | MISSING |
| Carboxylate)-deamination | | |
| Spermidine synthesis | PRESENT | PRESENT |
| Acetoin, Butanediol synthesis | PRESENT | PRESENT |
| Nitric oxide synthesis | MISSING | INCOMPLETE |
| Hydrogen cyanide synthesis | MISSING | MISSING |
| 2,4-Diacetylphloroglucinol synthesis | MISSING | MISSING |
| Flagellar assembly | PRESENT | PRESENT |

TABLE 35
Klebsiella genes involved in plant growth promoting features

| Enzyme | | |
|---|---|---|
| name | Gene name | FASTA gene sequence |
| exopoly- | ppx | >ADJCKNHF_00078 Exopolyphosphatase |
| phosphatase | | ATGCCAATAAACGAAAAAACCCCTCGGCCGCAGGAGTTCGCTGC |
| [EC:3.6.1.11] | | GGTCGACCTTGGTTCGAACAGTTTTCATATGGTGATCGCCCGTGT |
| | | CGTCGACGGGGCAATGCAGATCATCGGTCGCCTGAAGCAGCGCG |
| | | TCCACCTGGCCGATGGCCTGGATGAAAACTCGGTGTTGAGCGAAG |
| | | AAGCGATTACTCGTGGGCTGAACTGCCTGTCGCTGTTTGCGGAAC |
| | | GTCTGCAGGGATTCTCCCCTTCCAGCGTTTGTATCGTCGGGACGC |
| | | ATACCCTGCGCCAGGCGGCTAACGCGGCGGATTTTCTCAAACGAG |
| | | CGGAAAAAGTTATCCCTTATCCCATCGAAATAATCTCCGGTAACG |
| | | AAGAAGCGCGTCTGATTTTTATGGGCGTCGAACATACGCAACCGG |
| | | AGCGCGGCCGTAAGCTGGTTATCGACATCGGCGGCGGTTCGACTG |
| | | AGCTCGTCATCGGCGAAGATTTCGAGCCCCGCCTGGTTGAAAGCC |
| | | GACGTATGGGCTGCGTAAGCTTCTCGCAGGCCTATTTCGCCGGCG |
| | | GCGTTATCAATAAAGAAAACTTCCAGCGCGCGCGCCTGGCGGCG |
| | | GTGCAGAAACTGGAAACCCTGGCCTGGCAGTTCCGTATTCAGGGG |
| | | TGGAACGTGGCGCTGGGCGCCTCTGGCACCATCAAAGCCGCGCA |
| | | CGAAGTGCTGGTCGCCATGGGGGAGAAAGACGGCTTCATTACCC |
| | | CGGAACGTCTTGAAATGCTGGTCAGTGAGCTTCTGAAGAATAAAA |
| | | ACTTCGACTCATTAAGCCTGCCGGGATTGTCGGAGGACCGCAAAG |
| | | CGGTATTCGCCCCGGGTCTGGCGATTTTGTGCGGGGTATTCGACG |
| | | CGCTGGCGATCAAAGAACTGCGCCTGTCCGACGGCGCCCTGCGCG |
| | | AAGGCGTGTTGTACGAGATGGAAGGTCGCTTCCGCCACCAGGAT |
| | | ATTCGCAGCCGTACGGCCCAGAGCCTGGCGAACCAGTACAATATC |
| | | GATCGCGAACAGGCGCGCCGGGTACTGGAGACTACCACTCAGAT |
| | | GCTGGAACAGTGGCAGGAGCAAAACCCGAAACTGGCTAACCCGC |
| | | ATCTGTCTGCCCTGCTGAAGTGGGCGGTAATGCTACATGAAGTGG |
| | | GGCTGAACATTAACCACAGCGGCATGCATCGCCACTCTGCCTATA |
| | | TCCTGCAAAATAGCGATCTGCCGGGCTTTAATCAGGAGCAGCAGA |
| | | CGCTGATGGCCACGCTGGTGCGCTATCACCGCAAAGCTATCAAGC |
| | | TTGATGATTTGCCGCGTTTTACCTTGTTCAAGAAAAAACAGTTCTT |
| | | GCCGCTGATCCAACTACTGCGTCTGGGCGTATTACTGAACAATCA |
| | | GCGTCAGGCGACCACTACGCCGCCAAAACTGACATTGAAGACAG |
| | | AAGCCAACCACTGGACGTTAATTTTCCCGCATGACTGGTTTAGCC |
| | | AGAATGCGTTGGTGCTGCTGGATCTGGAAAAAGAGCAACAGTAC |
| | | TGGGAAGGCGTGCCGGAATGGCTGCTGAAAATTACTGAAGAAGA |
| | | AGCGTAA |
| indolepyruvate | ipdC | >ADJCKNHF_00151 Indole-3-pyruvate decarboxylase |
| decarboxylase | | ATGCAACCGACTTACACCATTGGCGATTATCTGCTGGATCGTCTC |
| [EC:4.1.1.74] | | GTAGATTGTGGTATCGATCGCCTGTTCGGCGTACCTGGTGATTAC |
| | | AACTTACAGTTTCTCGATCACGTGATAGCCCATCAAGATTTGGGA |
| | | TGGGTCGGCTGTGCTAACGAACTGAACGCGGCCTACGCCGCAGA |
| | | CGGCTATGCGCGTATAAAAGGCGCTGGCGCGCTGCTAACCACCTA |
| | | CGGCGTAGGGGAGTTAAGCGCGTTGAATGGGGTGGCCGGTAGTT |
| | | ATGCGGAACATATCCCGGTGCTGCATATTGTGGGCGCCCCTTCCA |
| | | CTGGCGCGCAGCAGCGCGGTGAACTCCTGCACCATACGCTCGGCG |
| | | ATGGCGATTTCACCCATTTCTCGCGGATGAGCGAACAAATTACCT |
| | | GTACTCAGGCGACGCTAACGGCGGGCAACGCCTGTCATGAAATT |
| | | GACCGGGTATTAAGCGACATGCTGACCCACCACCGCCCAGGGTAT |
| | | CTGATGCTGCCTGCCGACGTGGCGAAAGCCCGTGCGGTGCCGCCG |
| | | GCCCGCGCGCTGGTCATTAAGGGCCCGGCGGCCGATGAAAACCA |
| | | GCTTGCCGGCTTTCGCGAGCACGCGGCCAAATTGTTGCGCAGCAG |
| | | TCGCCGCGTATCGCTGCTGGCGGATTTTCTCGCTCAGCGTTACGGT |
| | | CTGCAAAACACACTACAGCAGTGGGTTAAGTCCGCGCCTATTACC |
| | | CACGCCACCATGCTGATGGGGAAAGGGCTATTTGACGAACAAGG |
| | | CTCAGGTTTTGCCGGGACCTATAGCGGGATCGCCAGCGCGCCGCA |
| | | GACTCGCGAGGCGATGGAAAGCGCCGATGCCATCATCTGCGTGG |
| | | GTACGCGCTTTACCGATACGATTACCGCCGGCTTTACCCATCATTT |
| | | GCCAACGGAGAAAACCATCGAAATCCAGCCGTTCGCCGCGCGGG |
| | | TTGGCGATCACTGGTTCAGCCGGATCCCGATGGATCAGGCGCTGG |
| | | CGGCACTCATTGACGTTTCTGGCAAGCTGTCCGCCGAATGGAGCG |
| | | CGCCAGATATCATCGCGCCCGACGCTTCCGGCGCGCCGCAGGGG |
| | | AATCTGACGCAGAAAAGCTTCTGGAGTACGGTCGAGAAACAGCT |
| | | GCGGCCGGGCGACATTATTCTTGCCGACCAGGGGACCTCGGCATT |
| | | CGGCATCGCCTCATTAAAACTTCCCTCCCAGGCGACGCTGCTGGT |
| | | GCAGCCGCTATGGGGCTCAATCGGCTTTACGCTACCTGCCGCCTA |
| | | CGGCGCACAGACAGCCGCCGCGGACAGAAGGGTGGTATTAATCA |
| | | TTGGCGATGGCGCCGCCCAGCTCACTATCCAGGAGATGAGCTCAA |
| | | TGCTGCGCGATAAGCAAAAGCTGTTAATTTTGCTGTTGAATAATG |
| | | AGGGCTACACCGTCGAGCGCGCGATTCACGGGCCAGAACAGCGC |
| | | TACAACGATATCGCATTGTGGGACTGGAGCCGCTTCCCCGATGCT |
| | | TTTGCGCCGGATACGCCTTCCCGCTGTTGGCGGGTAACGCAAACC |
| | | GGCGAATTGGCGGAGGCGATGGCCGATAGCATTAATTCCGATAA |
| | | GTTAACGATGGTCGAAGTCATGCTGCCGAAAATGGATATTCCTGA |
| | | TTTCTTACGCGCGGTCACCCAGGCGCTGGAAAATCGCAACAACCG |
| | | CGGCTAA |
| polyketide | Atu3672 | >ADJCKNHF_00178 3-oxoacyl-[acyl-carrier-protein] synthase 1 |
| synthase | | ATGAAACGTGCAGTGATTACTGGCTTGGGCATCGTTTCCAGCATC |
| | | GGTAATAACCAGCAGGAAGTCCTGGCATCTCTGCGTGAAGGACG |
| | | TTCAGGGATCACTTTCTCTCAGGAGCTGAAGGATTCAGGAATGCG |
| | | TAGCCACGTGTGGGGCAACGTCAAACTGGATACCACGGGCCTCAT |
| | | TGACCGCAAAGTAGTTCGCTTTATGAGCGATGCATCTATCTATGC |
| | | TTATCTGTCCATGGAGCAGGCGGTAGCCGACGCAGGTCTGGCGCC |
| | | GGAAGCATACCAGAACAACCCGCGTGTCGGCCTGATTGCAGGTTC |
| | | CGGCGGCGGCTCCCCGAAATTCCAGGTCTTCGGCGCCGATGCCAT |
| | | GCGTAGCCCGCGTGGTCTGAAAGCCGTGGGCCCATACGTTGTGAC |
| | | TAAAGCGATGGCTTCCGGCGTATCTGCCTGCCTCGCCACGCCGTT |
| | | TAAAATCCACGGCGTCAACTACTCTATTAGCTCCGCTTGCGCCAC |
| | | TTCCGCACACTGCATCGGTAACGCGGTAGAACAGATTCAGCTGGG |
| | | CAAACAGGACATCGTCTTTGCCGGCGGCGGCGAAGAGCTGTGCT |
| | | GGGAAATGGCTTGTGAATTCGACGCCATGGGCGCGCTGTCCACTA |
| | | AATACAACGATTCTCCGGACAAAGCGTCCCGTACCTATGATGCGA |
| | | ACCGCGACGGTTTCGTTATCGCGGGCGGCGGCGGCATGGTCGTGG |
| | | TGGAAGAGCTGGAACACGCGCTGGCGCGCGGCGCGCATATCTAT |
| | | GCGGAAATCGTCGGTTACGGCGCGACTTCCGATGGCGCGGACAT |
| | | GGTTGCTCCATCTGGCGAAGGCGCAGTGCGCTGCATGCAGATGGC |
| | | GATGCACGGCGTTGATACCCCGATCGACTACCTGAACTCCCACGG |
| | | CACTTCGACTCCGGTAGGCGACGTGAAAGAGTTGGGCGCTATCCG |
| | | CGAAGTCTTCGGCGACAACAGCCCGGCTATCTCCGCCACCAAAGC |
| | | GATGACCGGCCACTCTCTGGGCGCCGCAGGCGTACAGGAAGCCA |
| | | TCTACTCTCTGCTGATGCTGGAGCACGGTTTTATCGCGCCAAGCA |
| | | TCAACATTGAAGAGATGGACGAGCAGGCTGCCGGCCTTAACATC |
| | | GTCACCAAGCCGACCGATGCTCAGTTGACCACCGTGATGTCCAAC |
| | | AGCTTCGGCTTCGGCGGCACCAACGCGACCCTGGTTATGCGTAAA |
| | | TACAACGCCTAA |
| tryptophan | trpA | >ADJCKNHF_00747 Tryptophan synthase alpha chain |
| synthase | | ATGGAACGTTACGAGACGCTATTTGCACAGTTAAAAAACCGCCA |
| alpha chain | | GGAAGGCGCCTTTGTTCCCTTCGTCACCCTCGGCGATCCGGGGCC |
| [EC:4.2.1.20] | | GGAACAGTCGCTGAAAATCATCGATGCGCTGATTGAAGCCGGCG |
| | | CCGATGCGCTGGAACTGGGGATCCCCTTCTCCGACCCGCTGGCCG |
| | | ACGGCCCGACGATTCAGGGCGCCACATTGCGCGCTTTTGCCGCCG |
| | | GCGTTACCCCGGCGCAGTGCTTCGAGATGCTGGCGGCGATCCGCC |
| | | ACAAGCATCCGACCATTCCGATCGGCCTGTTGATGTACGCGAACC |
| | | TGGTGTTCAGCCCGGGGATTGATGAGTTCTACGCCGAATGCGCGC |
| | | GTGTCGGCGTGGATTCCGTGCTGGTCGCCGACGTGCCGGTCGAAG |
| | | AGTCCGCCCCGTTCCGTCAGGCAGCGCTGCGCCATAACATTGCGC |
| | | CGATTTTCATCTGCCCGCCAAATGCCGATGACGATTTACTGCGCC |
| | | AGATTGCCTCCTATGGCCGCGGCTATACCTACCTGCTATCGCGCG |
| | | CTGGCGTGACAGGCGCGGAAAACCGCGCCGCGCTGCCGCTGCAT |
| | | CATCTGATTGAAAAATTGGCGGAATATAACGCCGCGCCGCCGCTG |
| | | CAGGGCTTTGGTATCTCGGCGCCGGAGCAGGTTTCGGCCGCTATT |
| | | GACGCCGGTGCCGCCGGGGCAATATCCGGCTCGGCCATCGTCAA |
| | | GATCATCGAGCGCAACCTTGAACAGCCGCAAAAAATGCTCGAAG |
| | | AGCTAAAAACCTTCGTACAGAGTCTGAAAGCGGCGACCAAAACC |
| | | GCCTGA |
| tryptophan | trpB | >ADJCKNHF_00748 Tryptophan synthase beta chain |
| synthase beta | | ATGAGCACTTTACTGAACCCGTATTTCGGCGAATTCGGCGGTATG |
| chain | | TACGTTCCGCAGATCCTGATGCCCGCCCTGCGCCAGCTGGAAGAG |
| [EC:4.2.1.20] | | GCTTTCGTCAGCGCGCAAAAAGATCCTGAGTTTCAGGCCGAATTC |
| | | ACCGACCTGCTGAAAAACTACGCGGGCCGCCCGACGGCGCTGAC |
| | | CAAATGCCGCAATCTGACCGAAGGCACCCGCACCACGCTGTACCT |
| | | CAAGCGTGAAGATCTGCTCCACGGCGGCGCGCACAAAACCAACC |
| | | AGGTGCTGGGCCAGGCGCTACTGGCCAAACGTATGGGTAAAACG |
| | | GAAATTATCGCCGAAACCGGCGCCGGCCAACACGGCGTCGCCTC |
| | | GGCGCTGGCCAGCGCCCTGCTCGGTCTGAAATGCCGCATCTATAT |
| | | GGGCGCCAAAGACGTCGAGCGGCAGTCGCCGAACGTGTTCCGCA |
| | | TGCGCCTGATGGGTGCGGAAGTCATTCCAGTGCACAGCGGTTCCG |
| | | CCACCCTGAAAGATGCCTGTAACGAAGCGCTGCGCGACTGGTCCG |
| | | GCAGCTACGAGAAGGCGCACTACATGCTCGGCACCGCCGCTGGC |
| | | CCGCACCCATTCCCGACTATCGTGCGTGAATTCCAGCGCATGATC |
| | | GGCGAAGAAACCAAAGCGCAGATCCTTGAGAAAGAAGGACGCCT |
| | | GCCGGACGCGGTGATCGCCTGCGTTGGCGGTGGTTCTAACGCCAT |
| | | CGGCATGTTCGCTGATTTCATTGATGAATCCCGCGTTGGCCTGATT |
| | | GGCGTCGAGCCTGCCGGCCACGGTATCGAAACCGGCGAGCACGG |
| | | CGCGCCGCTGAAGCATGGTCGCGTCGGCATTTATTTCGGCATGAA |
| | | GTCGCCGATGATGCAAACCTCCGACGGGCAGATTGAAGAATCTTA |
| | | TTCTATCTCCGCCGGGCTGGATTTCCCGTCAGTGGGGCCTCAGCA |
| | | TGCGTTCCTTAATAGCACCGGGCGCGCTGAGTATGTGTCAATTAC |
| | | CGACAACGAAGCGCTGGATGCCTTTAAAGCGCTCTCCCGCCATGA |
| | | AGGCATCATTCCGGCGCTGGAGTCGTCGCACGCTCTGGCGCACGC |
| | | GCTGAAGATGATGCGCGAGAATCCGGATAAAGAGCAGCTGCTGG |
| | | TGGTTAACCTGTCCGGCCGCGGCGATAAAGACATCTTCACCGTAC |
| | | ACGACATTTTGAAAGCGCGAGGGGAAATCTGA |
| indole-3- | trpC | >ADJCKNHF_00749 Tryptophan biosynthesis protein TrpCF |
| glycerol | | ATGCAGACCGTTTTAGCAAAAATCGTTGCCGACAAAGCGATTTGG |
| phosphate | | GTAGAAGCCCGCAAACAACAACAACCGTTAGCCAGTTTTCAGAA |
| synthase | | TGACATTGTGCCGTCCGAACGCCATTTTTACGATGCGCTGGCCGG |
| [EC:4.1.1.48] | | CGCCCGCACCGCTTTTATTCTCGAGTGCAAAAAGGCGTCGCCGTC |
| | | GAAGGGACTGATTCGGGAAGATTTCGACCCGGCGACCATCGCCG |
| | | GGGTTTATAAGCACTACGCCTCGGCAATTTCGGTACTGTGCGATG |
| | | AGAAATATTTTCAGGGCAGCTTTGATTTTCTGCCGATCGTCAGCA |
| | | AAGTCGCGCCGCAGCCGATTCTGTGTAAAGACTTCACCATCGACC |
| | | CTTACCAGATTTATCTCGCGCGTTACTACCAGGCCGATGCCTGCCT |
| | | GCTGATGCTTTCGGTGCTCGATGACGATCAATATCGCCAGCTCGC |
| | | CGCCGTCGCCCACAGTCTCAATATGGGCGTCCTGACCGAGGTCAG |
| | | CAATGAAGAGGAGCTGGAGCGCGCGATTGCGCTGAAGGCCAAAG |
| | | TCGTCGGCATTAACAACCGCGATCTGCGCGATATGTCGATTGATC |
| | | TGAACCGCACGCGGCAATTGGCGCCGCGTCTCGGCCCGGATGTCA |
| | | CCGTGATCAGCGAATCCGGAATCCATACCTATGGCGAAGTCCGCG |
| | | AGCTTAGCCACTTCGCCAACGGCTTCCTGATTGGCTCGGCGCTGA |
| | | TGGAACAACCGGACCTCAGCGCTGCGGTGAAGCGCGTGCTGTTA |
| | | GGTGAGAACAAGGTCTGCGGCCTGACTCGTCCGCAGGATGCGCA |
| | | AGTCGCCTGGGAGGCGGGCGCCATCTACGGCGGCCTGATTTTCGT |
| | | CGATAGTTCGCCGCGTGCCGTTAACGATCAGCAGGCACAGGCGGT |
| | | AATGGCCGCCGCGCCGCTTAGCTACGTTGGCGTGTTCCGTGATGC |
| | | AGCCGTTGAAGAGGTCGTCACCCGCGCGACCAAATTGAAACTCG |
| | | CCGCGGTCCAGCTTCATGGGAGCGAAGATCAGGCCTGGATTGATG |
| | | CCCTGCGCGCGGCGCTGCCGGAGCAGATCCAAATCTGGAAGGCG |
| | | CTGAGCGTCGGCGAAAGTTTACCGCCGCGCAACCTGAACCATGTG |
| | | ACCAGGTATGTCTTTGACAACGGTCAGGGCGGGAGCGGTCAACG |
| | | TTTCGACTGGTCGCTGCTGCAGGGCCAGGACCTGCGTAACGTGAT |
| | | GCTGGCAGGCGGCCTCAGCGCCGATAACTGCGTAGAAGCCGCGA |
| | | AAAGCGGCTGTGCCGGACTCGATTTCAACTCAGGCGTAGAGTCGC |
| | | AGCCGGGAATAAAAGAGGCAAGCCTGGTGGCTGCCGTTTTCCAG |
| | | ACGCTGCGCGCATATTAA |
| anthranilate | trpD | >ADJCKNHF_00750 Bifunctional protein TrpGD |
| phosphoribos | | ATGGCCGACATCCTGCTGCTCGATAATATCGACTCCTTTACCTATA |
| yltransferase | | ACCTCGCCGACCAGCTGCGGGCTAACGGCCACAACGTGGTTATTT |
| [EC:2.4.2.18] | | ATCGTAATACCGTACCGGCACAATCGCTGATTGAACGTATCGGCA |
| | | CCATGGATAATCCGGTGCTGATGCTCTCTCCGGGGCCGGGAACCC |
| | | CAAGCGAAGCCGGCTGCATGCCCGAGCTGCTGACCCGCCTGCGC |
| | | GGCAAGTTACCGATTATCGGTATCTGCCTCGGCCACCAGGCCATC |
| | | GTGGAAGCCTACGGCGGCTATGTCGGCCAGGCGGGAGAAATCCT |
| | | TCACGGCAAAGCGTCAAGCATTGAGCATGACGGCCAGGCGATGT |
| | | TCGCCGGTCTCGCCAACCCGCTGCCGGTGGCCCGCTACCATTCTC |
| | | TTGTCGGCAGCAATATTCCGGCCGGGCTCACCATTAACGCCAATT |
| | | TCAACGGTATGGTGATGGCGGTTCGCCACGACGCCGATCGCGTCT |
| | | GCGGCTTCCAGTTCCATCCGGAATCGATTCTGACTACCCAAGGAG |
| | | CGCTACTGCTGGAGCAAACGCTGGCATGGGCGCTGCAGAAACTG |
| | | GAGCAGACCAATGTCGTACAGCCGATTCTGGAAAAACTGTACCA |
| | | GGCCGAGACGCTGAGCCAGCAGGAGAGCCACGTGCTGTTTTCCG |
| | | CCGTCGTGCGCGGCGAAGTGAAGCCGGAACAGCTGGCCGCCGCG |
| | | CTGGTAAGCATGAAAGTGCGCGGCGAACAACCACAGGAAATTGC |
| | | CGGCGCCGCTACCGCGCTGCTGGAAAACGCCGCGCCGTTCCCGCG |
| | | CCCGGATTATCAGTTTGCCGATATCGTCGGTACCGGCGGCGATGG |
| | | CAGCAACAGTATCAATATCTCAACCGCCAGCGCCTTTGTCGCCGC |
| | | CGCCTGCGGTTTAAAAGTGGCGAAACACGGTAACCGCAGCGTCTC |
| | | CAGTAAATCGGGTTCGTCCGACCTGCTGGCCGCGTTTGGCATCAA |
| | | CCTGGATATGAATGCCGATAAATCGCGCGCCGCGCTGGATGAACT |
| | | GGGCGTCTGCTTTCTGTTCGCGCCGAAATACCACACCGGTTTCCG |
| | | CCACGCGATGCCGGTCCGCCAGCAGTTGAAAACGCGCACCCTGTT |
| | | TAACGTGTTGGGGCCGCTTATCAACCCGGCGCACCCGCCGCTGGC |
| | | GTTGATTGGCGTCTACAGCCCGGAACTGGTGTTGCCGATTGCAGA |
| | | AACCTTGCGCGTACTCGGTTATCAACGCGCCGCGGTGGTACACAG |
| | | CGGCGGCATGGACGAAGTCTCGTTGCATGCGCCGACCGTGGTCGC |
| | | CGAACTCAATAACGGCGAAATCCAGAGCTATCAGCTGACCGCCG |
| | | CCGACTTCGGCCTGACGCCTTATCATCAGGAGCAACTGGCCGGCG |
| | | GCACGCCGGAAGAAAACCGTGACATTCTCACGCGCTTGCTACAA |
| | | GGTAAAGGTGAAGCCGCTCATGAAGCCGCCGTGGCCGCCAACGT |
| | | CGCCATGTTGATGCGTTTACACGGGCATGAAGACCTGAAAGCCAA |
| | | CGCCCGGCAGGTACTGGATGTGCTGCACAGCGGAGCGGCCTATG |
| | | ACAGAGTGACCGCATTAGCGGCAAGAGGGTAA |
| anthranilate | trpE | >ADJCKNHF_00751 Anthranilate synthase component 1 |
| synthase | | ATGCAAACATCAAAACCAGCGCTCGAGCTTCTCACCAGCGACGCC |
| component I | | ATCTATCGCGAAAACCCGACGGCGTTATTCCATCAATTATGCGGC |
| [EC:4.1.3.27] | | GCGCGTCCGGCAACCTTGCTGCTGGAATCGGCCGACATCGATAGT |
| | | AAAGACGATTTAAAAAGCCTGCTGCTGGTCGATAGCGCCCTGCGC |
| | | ATTACCGCTCTTGGCAATACCGTCACTGTCCAGGCGCTTTCAGCG |
| | | AACGGCGCCGCGCTGCTTGAACTGCTGGATAACGCATTGCCGTCC |
| | | GGCATTGAGAATCTGCGCCAGCCGAACAGCCGGGTACTCACCTTC |
| | | CCACCGGTTAGCGCCCTGCTGGATGAAGACGCTCGCCTGTGTTCG |
| | | TTATCGGTGTTCGATGCGTTTCGTCTGTTACAGGATTTGGTCAGCG |
| | | TCCCGCAGGCCGAGCGCGAAGCGATGTTCTTCGGCGGCCTGTTCG |
| | | CTTACGACCTGGTGGCCGGTTTTGAGGATTTACCACCGCTCAATA |
| | | CCGATACCGCCTGCCCGGATTACTGTTTCTACCTGGCGGAGACGC |
| | | TGCTGGTCATCGATCACCAGACCAAAAGCACCCGCATTCAGGCGA |
| | | GCCTGTTCACGCCGCTGGAGAATGAGAAACAGCGTCTTCTGCAAC |
| | | GTATCGCCCAGCTTCGCCAGCAGTTAAATGAACCGCCCGCGCCGC |
| | | TGCCGGTGACAACGGTTGCCGAAATGCGCTGCGACGTCGATCAG |
| | | AGCGATGAAGAGTACGGCGCCGTGGTGCGCAAAATGCAGCGCGC |
| | | CATTCGCGCGGGCGAAATTTTCCAGGTCGTGCCGTCGCGCCGCTT |
| | | CTCCCTCCCCTGCCCGTCGCCGCTGGCCTCGTACGATGTGCTGAA |
| | | AAAGAGCAATCCCAGCCCGTATATGTTCTTTATGCAGGATAACGA |
| | | TTTCACCCTGTTCGGCGCATCGCCGGAAAGCTCGCTGAAATACGA |
| | | CGCCGTCAGCCGCCAGATTGAGATCTACCCCATTGCCGGCACCCG |
| | | TCCGCGCGGCCGCCGCGCCGATGGTTCGCTGGATCGCGATCTCGA |
| | | TAGCCGTATTGAGCTGGAGATGCGTACCGACCACAAAGAACTTTC |
| | | TGAACATCTGATGCTGGTCGACCTGGCCCGTAACGATCTGGCCCG |
| | | CATCTGTACCCCGGGTAGCCGCTACGTGGCGGATTTAACCAAAGT |
| | | CGATCGCTACTCCTTTGTGATGCATCTGGTTTCCCGCGTGGTCGGC |
| | | GAACTGCGCCGCGATCTTGATGTCCTGCACGCCTACCGCGCCTGC |
| | | ATGAATATGGGCACCCTTAGCGGCGCGCCGAAAGTTCGCGCCATG |
| | | CAGCTAATCGCCGCCGCGGAAGGCAAGCGTCGCGGTAGCTACGG |
| | | CGGCGCGGTCGGCTACTTTACCGCCCATGGCGACCTCGATACCTG |
| | | CATCGTGATCCGCTCAGCCTACGTTGAGGATGGCATTGCTACCGT |
| | | GCAGGCGGGCGCCGGTATCGTGCTCGATTCCGTTCCGCAATCCGA |
| | | GGCGGATGAAACCCGTAATAAAGCTCGCGCCGTGCTGCGCGCCA |
| | | TTGCTCAGGCCCACCACGCGAAGGAGACTTTCTAA |
| alkaline | phoA, phoB | >ADJCKNHF_00859 Primary amine oxidase |
| phosphatase | | ATGGCAAACGGCTTGATGTTTTCCCCTCGTAAAACCGCGCTGGCG |
| [EC:3.1.3.1] | | CTGGCTGTCGCGGTGGTTTGCGCCTGGCAATCACCGGTCTTCGCT |
| | | CACGGTAGCGAAGCGCACATGGTGCCGTTGGATAAAACGCTGCA |
| | | GGCGTTCGGCGCCGATGTGCAGTGGGATGACTACGCGCAAATGTT |
| | | CACCCTGATAAAAGACGGCGCTTACGTCAAAGTAAAACCCGGCG |
| | | CCAAAACGGCGATCGTCAACGGTAACCCCCTTGATCTACAGGTGC |
| | | CGGTAGTAATGAAGGAAGGTAAAGCCTGGGTCTCCGATACCTTTA |
| | | TCAACGATGTATTCCAGTCCGGTCTCGATCAGACCTTCCAGGTAG |
| | | AAAAACGCCCTCACCCGTTAAATTCGCTCTCGGCGGCGGAAATCG |
| | | GTGAAGCAGTGACCATTGTTAAAGCCGCGCCGGAGTTCCAGCCG |
| | | AATACCCGCTTTACTGAGATTTCCTTACACGAGCCGGACAAGGCG |
| | | GCTGTATGGGCCTTTGCCCTGCAGGGAACGCCCGTTGATGCACCC |
| | | CGCACCGCCGATGTGGTGATGCTCGATGGCAAACATGTCATTGAA |
| | | GCCGTCGTCGATCTGCAAAACAAAAAAATCCTCTCATGGACGCCG |
| | | ATTAAAGGCGCCCACGGGATGGTGCTGCTTGATGACTTCGTCAGC |
| | | GTGCAGAACATTATCAATGCCAGCAGCGAGTTCGCTGAGGTGCTG |
| | | AAAAAGCACGGTATTACCGATCCCAGTAAGGTGGTCACCACCCC |
| | | GCTCACCGTCGGCTACTTTGATGGCAAAGATGGCCTGCAGCAGGA |
| | | TGCACGTCTGCTGAAAGTCGTCAGTTATCTGGATACCGGCGACGG |
| | | CAACTACTGGGCGCACCCGATTGAAAACCTGGTGGCGGTGGTCG |
| | | ACCTTGAAGCGAAGAAAATCATCAAAATCGAAGAAGGCCCGGTG |
| | | CTCCCGGTACCGATGGAGCCCCGTTCTTACGATGGTCGCGACCGC |
| | | AACGCCCCGGCGGTGAAACCGCTGGAGATAACCGAACCGGAAGG |
| | | CAAAAACTACACGATCACCGGCGATACCATTCACTGGCGGAACT |
| | | GGGATTTTCATCTGCGCCTGAACTCGCGCGTCGGGCCCATTCTCTC |
| | | GACGGTGACTTACAACGATAACGGCACAAAACGCCAGGTAATGT |
| | | ATGAAGGTTCCCTCGGCGGGATGATCGTCCCCTACGGCGACCCTG |
| | | ACGTCGGCTGGTATTTCAAAGCCTATCTGGACTCCGGCGATTACG |
| | | GCATGGGCACCCTAACATCGCCTATTGTTCGCGGTAAAGATGCGC |
| | | CGTCAAATGCAGTACTGCTGGACGAAACTATCGCCGATTACACCG |
| | | GCAAACCGACCACTATTCCAGGCGCGGTCGCCATATTCGAACGCT |
| | | ATGCCGGGCCAGAATATAAGCACCTGGAAATGGGCAAGCCCAAC |
| | | GTCAGCACCGAGCGCAGGGAACTGGTGGTGCGCTGGATCAGTAC |
| | | CGTCGGGAACTATGATTATATCTTTGACTGGGTGTTCCACGACAA |
| | | TGGCACCATCGGTATCGATGCCGGCGCCACCGGCATTGAAGCCGT |
| | | TAAAGGCGTGAAGGCGAAGACCATGCACGACCCCAGCGCCAAAG |
| | | AGGATACCCGCTACGGGACGCTGATCGACCATAATATTGTCGGCA |
| | | CCACCCACCAGCATATTTATAATTTCCGCCTCGATCTCGACGTGG |
| | | ACGGCGAAAACAACACCCTGGTGGCGATGGATCCTGAAGTGAAG |
| | | CCAAACACCGCCGGCGGCCCGCGCACCAGCACCATGCAGGTGAA |
| | | TCAGTACACAATCGATAGCGAGCAGAAAGCGGCGCAGAAATTCG |
| | | ACCCTGGCACTATCCGCCTGCTGAGCAACACCAGCAAAGAGAAC |
| | | CGCATGGGTAACCCGGTCTCTTACCAGATTATCCCTTATGCCGGC |
| | | GGCACGCATCCGGCGGCGACCGGCGCGAAGTTCGCCCCGGACGA |
| | | GTGGATATATCATCGTCTGAGCTTTATGGATAAACAGCTGTGGGT |
| | | GACGCGTTACCACCCGACAGAGCGTTATCCGGAAGGGAAATACC |
| | | CTAACCGTTCCGCCCAGGATACCGGCTTAGGCCAGTACGCGAAGG |
| | | ATGATGAGTCGCTGACAAACCACGACGACGTCGTGTGGATCACC |
| | | ACCGGCACCACCCACGTCGCGCGCGCCGAAGAGTGGCCAATTAT |
| | | GCCGACCGAGTGGGCGCACGCGCTGCTCAAGCCGTGGAACTTCTT |
| | | TGACGAAACCCCAACGCTTGGCGAGAAGAAAAAGTAA |
| pfam01011 | pfam01011 | >ADJCKNHF_00899 Quinate/shikimate dehydrogenase (quinone) |
| | | ATGGCTACAGGCAACGTGCCGCGCGGATTCCCCCGGATCCTGCAG |
| | | TGGCTACTCGCCGGACTGATGTTAATCATCGGTCTGGCAATCGGC |
| | | ATTTTAGGCGCCAAACTGGCAAGCGTCGGCGGCACCTGGTATTTC |
| | | GCCATTATGGGACTGGTGATGGTTATCGCCTCCCTGCTTATTTTCC |
| | | GCAATCGGCGCGGCGGCATCGTTCTTTATGCCGTCGCCTTCGCGG |
| | | CTTCCATTGTTTGGGCTATCAGCGACGCCGGCTGGACCTACTGGC |
| | | CGCTGTTCTCACGCCTGTTTGCGCTTGGCGTTCTGGCTTTTCTGTG |
| | | CGCCATTGTTTGGCCTTTTCTTTCCAGCGCACCGGCAAAAAAAGG |
| | | CGCGGCCTTCGCCCTCGCGGGCGTGCTGGCCGTGGCACTGCTGGT |
| | | CAGTTTTGGTTGGATGTTTAAATCCCAGCCGCTGGTCAGCGCCAG |
| | | CGAAGCGGTGCCGGTAAAACCGGTACAGCCAGGTGAAGAACAAA |
| | | AGAACTGGCAGCACTGGGGTAATACCACTCACGGCGACCGTTTCG |
| | | CCGCGTTGGATCAGATCAATAAAAACAATATCAACCAGCTACAG |
| | | GTTGCCTGGATTGCCCATACCGGCGATATTCCGCAGAGTAACGGT |
| | | TCCGGTGCGGAAGACCAGAATACGCCGCTGCAGATTGGCGATAC |
| | | GCTTTATGTTTGTACGCCATATAGCAAAGTCCTGGCGCTGGATGT |
| | | CGATAGCGGCAAAGAAAAATGGCGCTACGACTCGAAAGCGACGG |
| | | CGCCTAACTGGCAACGTTGCCGCGGTTTAGGCTACTACGAAGATA |
| | | GCCAGGCGCAGGCTAATGCCGTGGCCGGGACTCAGCCTGCCGCCT |
| | | GTGCCCGCCGCCTGTTCCTGCCGACTACCGATGCGCGCCTGATCG |
| | | CAATCGATGCCGATACCGGTAAAGCCTGTGAAGCATTTGGTGAAC |
| | | ACGGTACCGTCGATCTCAGCGTCGGCATGGGGGAAATCAAACCT |
| | | GGTTATTATCAGCAGACCTCTACCCCGCTGGTGGCCGGTAACCTT |
| | | GTTGTCGTTGGTGGTCGCATCGCGGATAACTACTCCACCGGTGAA |
| | | CCGCCGGGTGTAGTTCGCGCTTTTGACGTGCATACCGGTAAACTG |
| | | GCCTGGGCCTGGGATCCGGGTAACCCGCAGCTGACCGGCGTACC |
| | | GCCGGAAGGACAAACCTACACACGCGGGACGCCGAACGTCTGGT |
| | | CCGCGATGTCTTACGATGCCAAACTGAATCTCATTTATCTGCCAA |
| | | CCGGTAACGCCACGCCAGATTTCTTCGGCGGCGAACGTACGGCAC |
| | | TGGATGATAAATACAGCTCATCAATCGTTGCGGTCGATGCCGCCA |
| | | CCGGTAAAGTGCGCTGGCATTTCCAGACCACCCATCACGATCTGT |
| | | GGGATTTCGACCTGCCTTCGCAACCACTGCTGTACGATCTGCCGG |
| | | ACGGTAAAGGCGGTACGACGCCGGTACTGGTGCAAACCAGCAAG |
| | | CAGGGCATGATCTTCATGCTTAACCGCGCGACGGGTGAACCGGTG |
| | | GCGAAAGTGGAAGAACGCCCTGTCCCGGCCGGCAACGTCAAAGG |
| | | TGAACGCTACTCGCCGACGCAGCCCTACTCCGTCGGCATGCCGAT |
| | | GATCGGCAACGAAACGTTGAAAGAATCGGATATGTGGGGCGCCA |
| | | CCCCGGTTGACCTGCTGCTGTGCCGTATTCAGTTCAAAGAGATGC |
| | | GCCATCAGGGTGTCTTTACACCGCCGGGTGAAGACCGCTCCTTGC |
| | | AGTATCCGGGCTCGCTTGGCGGAATGAACTGGGGCAGCGTGTCG |
| | | GTCGATCCGAATAACGGCCTGATGTTCGTCAATGACATGCGCCTT |
| | | GGACTGGCTAACTACATGGTACCGCGAGCGAAAGTGGCTAAAGA |
| | | TGCCAGCGGTATCGAAATGGGTATCGTACCGATGGAAGGTACGC |
| | | CGTATGGCGCAATGCGCGAGCGTTTCCTGTCGCCGCTGGGCATCC |
| | | CCTGCCAGAAGCCGCCGTTCGGTACCATGTCGGCGGTTGATCTGA |
| | | AAAGCGGTAAGCTGGTCTGGCAGGTTCCGGTAGGTACGGTAGAA |
| | | GATACCGGTCCGCTGGGTATCCGCATGCATATGCCGATCCCTATC |
| | | GGCATGCCAACGCTGGGAGCTTCGCTGTCTACCCAGTCTGGCCTG |
| | | CTGTTCTTCGCCGGCACCCAGGATTTCTATCTGCGCGCCTTTGATA |
| | | CCGCCAACGGCAAAGAGATTTGGAAATCTCGCCTGCCGGTGGGC |
| | | AGCCAATCCGGCCCGATGACCTACGTTTCACCGAAAACCGGTAAG |
| | | CAGTACATCATTATCAACGCCGGCGGCGCGCGCCAGTCTCCGGAT |
| | | CGCGGCGATTACATCATCGCTTACGCTTTAGCGGATAAGCAGTAA |
| nitrate | narI, narV | >ADJCKNHF_00973 Respiratory nitrate reductase 2 gamma chain |
| reductase | | ATGATACAGTACTTAAACGTCTTCTTTTACGATATCTACCCTTATC |
| gamma | | TCTGCGGCACCGTGTTCCTGGTGGGCAGTTGGCTGCGCTATGACT |
| subunit | | ACGGGCAGTATACCTGGCGCGCGTCATCCAGCCAGATGCTCGATA |
| [EC:1.7.5.1 | | AACGGGGCATGGTGCTGTGGTCGAACTTATTCCATATCGGCATCC |
| 1.7.99.-] | | TCGGCATTTTCTTCGGCCACTTCTTCGGGATGCTGACGCCGCACTG |
| | | GGTCTATTCATGGTTTCTGCCGATGTCGCAGAAACAGGTGATGGC |
| | | GATGGTGCTGGGCGGTGTCTGCGGCGTACTGACCCTGGTCGGCGG |
| | | TATTGGCCTGCTGGCGCGCCGCCTGACCAACCCGCGCATTCGCGC |
| | | CACCTCCACCACTGCGGATATTCTGATCCTGTGCATTCTGCTGATT |
| | | CAGTGCGCGCTGGGGCTGACGACCATCCCGTTCTCCGCCCAGCAT |
| | | CCGGACGGCAGCGAAATGCTGAAACTGGTGGATTGGGCGCAGGC |
| | | GGTAGTCACCTTCCACGGCGGCGCATCGGCGCATCTGGACGGCGT |
| | | CGCGTGGGTGTATCGCGTCCATCTGGTGCTGGGAATGACTATCTT |
| | | CCTTATCTTCCCGTTCACCCGGCTGGTTCACGTCTGGAGCGCGCCG |
| | | ATAGAGTATTTCACCCGCCGCTACCAGGTGGTGCGCTCCCGGCGC |
| | | TGA |
| nitrate | narJ, narW | >ADJCKNHF_00974 putative nitrate reductase molybdenum |
| reductase | | cofactor assembly chaperone NarW |
| molybdenum | | ATGCGGATCCTCAAAGTCATTGCTCTGTTGCTGGAGTATCCCGAC |
| cofactor | | GACGCGCTGTGGGATAACCGCGAGGAGGCGCTGGCGCTGGTCAG |
| assembly | | CGCCGATGCGCCTGCCCTGACGCCGTTTGTTAGGCGATTACTGGC |
| chaperone | | GGCGCCGCTGCTCGACCGCCAGGCCGAATGGTGTGAGGTATTTGA |
| NarJ/NarW | | ACGCGGTCGCGCCACCTCGCTGTTGCTGTTTGAACACGTTCACGC |
| | | CGAATCACGCGATCGCGGCCAGGCGATGGTCGATCTGATGAACC |
| | | AGTATGAACAGGCGGGCCTGCAGATCGATTGTCGCGAACTGCCG |
| | | GACCACCTGCCTTTATATCTTGAATACCTCAGCATTCTCCCATTAG |
| | | CCGATGCCCGTGAAGGACTGCAGAACGTCGCGCCTATTCTGGCGT |
| | | TGCTCGGTGGACGCTTAAAGCAGCGCGAATGCGATTACTATCAAC |
| | | TCTTTGACACCCTTCTTGCGCTGGCGGATAGCCCACTCAATAGTG |
| | | ACAGTGTCACCAGGCAGGTAGCGAGTGAGAAGCGTGACGACACC |
| | | CGCCAGGCGCTGGATGCGGTGTGGGAAGAGGAACAGGTGAAGTT |
| | | TATTGAAGATAATGCAACCGCTTGCGATAGCTCGCCGATGCAAGC |
| | | TTATCAACGACGCTTTAGCCAGGATGTGGCGCCGCAGTACGTCGA |
| | | CATCCGCGCCGGAGGCCCGAAATGA |
| nitrate | narY, narH, | >ADJCKNHF_00975 Respiratory nitrate reductase 2 beta chain |
| reductase/ | nxrB | ATGAAAATACGTTCACAAGTCGGAATGGTGCTGAATCTGGATAA |
| nitrite | | ATGCATCGGATGCCATACCTGCTCCGTCACCTGTAAAAACGTCTG |
| oxidoreductase, | | GAGCAGCCGCGAAGGGATGGAATATGCGTGGTTCAACAACGTGG |
| beta | | AAACCAAGCCAGGTATTGGTTACCCCAAAAACTGGGAAGATCAG |
| subunit | | GACGAGTGGCAAGGCGGCTGGATCCGCGGTATCAGCGGCAAGCT |
| [EC:1.7.5.1 | | TACTCCGCGTCTGGGCAACCGCGTCAGCGTGTTGTCGAAGATTTT |
| 1.7.99.-] | | CGCCAACCCGGTGCTGCCGCAGATTGACGATTATTACGAACCCTT |
| | | TACCTACGATTATCAGCACCTGCACAACGCGCCGGAGGGCAAAT |
| | | ACTTGCCTACCGCCCGCCCGCGTTCGCTGATTAGCGGCGAACGGA |
| | | TGGATAAGATCACCTGGGGGCCAAACTGGGAGGAACTGCTCGGC |
| | | GGCGAGTTTGAAAAACGCGCCAAAGACCGTAACTTCGAAGCGAT |
| | | GCAAAAAGAGATGTACGGCCAGTTTGAAAACACCTTCATGATGT |
| | | ATCTGCCGCGTCTTTGCGAACATTGCCTCAATCCGAGCTGCGTAG |
| | | CGACCTGCCCAAGCGGCGCCATCTATAAGCGTGAAGAAGACGGT |
| | | ATCGTGCTGATCGACCAGGATAAATGCCGCGGCTGGCGGATGTGC |
| | | ATCAGCGGTTGTCCGTACAAAAAAATCTACTTTAATTGGAAGAGC |
| | | GGCAAATCGGAAAAATGCATCTTCTGCTATCCGCGTATTGAGTCC |
| | | GGGCAACCGACAGTCTGTTCAGAAACCTGCGTTGGCCGCATCCGC |
| | | TACCTTGGCGTGCTGCTGTACGACGCGGACCGTATCGAAGAAGCG |
| | | GCCAGCACTGAACATGAAACCGACCTCTACGAGCGCCAGTGTGA |
| | | TGTGTTCCTCAACCCCAACGATCCGGCGGTCATCGAAGAGGCGTT |
| | | GAAACAGGGTATTCCACATAACGTGATCGACGCCGCGCAGAAAT |
| | | CACCGGTGTATAAACTGGCGATGGACTGGAAGCTGGCGCTGCCG |
| | | CTGCACCCGGAATACCGCACCTTACCGATGGTCTGGTACGTGCCG |
| | | CCGCTGTCGCCGATTCAGTCGGTCGCCGACGCTGGCGGCCTGCCG |
| | | AGTAACGGTAACGTCCTGCCGGCGGTAGAAAGCCTGCGTATACC |
| | | GGTACAGTATCTGGCGAACCTGCTGAGCGCCGGTGATACCGGCCC |
| | | GGTCTTGCGCGCGTTAAAACGCATGATGGCGATGCGCCATTACAA |
| | | ACGTTCGCAAACCGTCGAAGGCGTGACCGATACCCGTGCGATTGA |
| | | AGAAGTGGGCCTCAGCGTCGAACAGGTGGAAGAGATGTATCGCT |
| | | ACCTGGCGATCGCCAATTATGAAGACCGCTTCGTGATCCCCACCA |
| | | GCCACCGCGAACTGGCGGAGGACGCCTTCCCTGAACGCAACGGC |
| | | TGCGGTTTTACCTTCGGCGATGGTTGCCATGGTTCTGATACTAAAT |
| | | TCAACCTGTTCAACAGTCGTCGCATCGACGCTATTGACGTCGGAG |
| | | ATGTTCGTCGACATGGGGAGGGAGAGTAA |
| nitrate | narG, narZ, | >ADJCKNHF_00976 Respiratory nitrate reductase 2 alpha chain |
| reductase/ | nxrA | ATGAGTAAATTACTGGATCGCTTTCGCTACTTTAAGCAGAAACGC |
| nitrite | | GAGACCTTTGCCAACGGCCACGGTCAGGTCTACGACAATAACCGT |
| oxidoreductase, | | GACTGGGAAGATAGCTACCGGCAACGCTGGCAATTCGACAAAAT |
| alpha | | TGTCCGCTCCACGCACGGTGTGAACTGTACCGGCTCCTGTAGCTG |
| subunit | | GAAAATTTATGTCAAAAACGGTCTGGTCACCTGGGAAACCCAGC |
| [EC:1.7.5.1 | | AGACCGATTATCCGCGCACCCGACCGGACCTGCCAAATCACGAA |
| 1.7.99.-] | | CCGCGCGGCTGTCCGCGCGGCGCCAGCTACTCCTGGTATCTCTAC |
| | | AGCGCCAACCGCCTGAAGTACCCGCTGGCGCGCAAACGGCTGAT |
| | | TGAGCTGTGGCGCGAAGCACTTACACAGCATCCCGACCCGGTACA |
| | | GGCCTGGGATAGCATCATGCAGGACCCCCTGAAAACCCGCAGCT |
| | | ACAAACAAATCCGCGGTAAAGGCGGTTTTGTTCGCTCCAGTTGGA |
| | | AAGAGTTAAATCAACTGATCGCCGCAGCTAACGTCTGGACCATCA |
| | | AAAACTACGGTCCGGACCGCGTCGCGGGCTTTTCGCCGATCCCGG |
| | | CGATGTCGATGGTTTCCTACGCCGCGGGTACCCGCTATTTATCGTT |
| | | GATCGGCGGCACCTGCCTGAGCTTTTATGACTGGTACTGCGATTT |
| | | ACCCCCCGCCTCGCCGATGACCTGGGGCGAACAGACCGACGTGC |
| | | CGGAATCCGCCGACTGGTATAACTCCAGCTACATTATTGCCTGGG |
| | | GTTCAAACGTCCCGCAGACCCGAACCCCGGACGCGCACTTCTTCA |
| | | CCGAAGTGCGCTACAAGGGCACCAAAACCATCGCCATTACTCCG |
| | | GATTATTCGGAAGTCGCCAAGCTCTGCGACCAGTGGCTGGCGCCG |
| | | AAGCAAGGTACCGACAGCGCGCTGGCGATGGCGATGGGCCACGT |
| | | GATCCTCAAAACCTTCCACCTCGATAACCCAAGCGATTACTTTCT |
| | | CAACTACTGCCGTACCTATACCGATATGCCGATGCTGGTAATGCT |
| | | CGACCGTCGTGACGACGGCAGCTATGCGCCTGGCCGTATGCTGCG |
| | | CGCCGCCGACCTGCTTGACGGGCTGGGAGAAAGTAATAACCCGG |
| | | AATGGAAAACCGTCGCCTATAACGCCGAAGGCGAGCTGGTCGCG |
| | | CCGAACGGATCCATCGGCTTTCGTTGGGGCGAAAAAGGCAAATG |
| | | GAATCTCGAACAACAGGCTAACGGCCGGGACGTCGAATTAAAAT |
| | | TGTCGCTGCTGGATATGCACGATAGCGTGGTGTCGGTCGGTTTCC |
| | | CCTACTTCGGCGGCAACGAAAACCCGCATTTCCGCAGCGTGAAGC |
| | | AGGAGCCGGTAACGCTTCATCAGCTGCCAGCTAAACAGCTACCGT |
| | | TGGCCAATGGTGAGAGCGCTTTGGTGGTGAGCGTGTATGACCTGG |
| | | TGCTGGCTAACTACGGTCTGGATCGCGGTCTTGGCGATGCCAATG |
| | | CCGCGCGCGACTTCGCGGATGTCAAAGCCTATACCCCGGCATGGG |
| | | CCGAGCAGATCACCGGCGTGCCGCGTCAACATATCGAACAAATC |
| | | GCCCGCGAGTTCGCCGATACGGCGCATAAAACCCATGGCCGTTCG |
| | | ATGATTATCCTCGGCGCCGGTGTGAACCACTGGTACCACATGGAT |
| | | ATGAACTACCGCGGGATGATCAACATGCTGGTGTTCTGCGGCTGC |
| | | GTCGGTCAGAGCGGCGGTGGCTGGTCGCACTATGTCGGCCAGGA |
| | | GAAACTGCGCCCGCAGACCGGCTGGCTGCCGCTGGCGTTCGCGCT |
| | | GGACTGGACTCGTCCGCCGCGGCAGATGAACAGTACCTCTTATTT |
| | | CTACAACCACGCCAGCCAATGGCGCTACGAAAAACTGACCGCTC |
| | | AGGAGCTGCTGTCGCCGCTGGCCGACGCCAGCAAATTCAGCGGC |
| | | CATATGATTGACTTCAACGTTCGCGCCGAGCGCATGGGCTGGCTG |
| | | CCGTCCGCGCCGCAGCTTAACGTTAATCCGCTGAGCATCAGGCAG |
| | | CAGGCCGAGGCCGCCGGGCTGTCACCGGCCGATTTCACTGTCCAG |
| | | TCGTTAAAAGCAGGCGATATTCGCTTTGCCGCCGAGCAGCCGGAT |
| | | AGCGGCAACAACCACCCGCGCAACCTGTTTATCTGGCGTTCGAAC |
| | | CTGCTGGGTTCGTCCGGTAAGGGCCATGAGTACATGCTCAAGTAC |
| | | CTGCTCGGTACCGATAACGGTATTCAGGGCGAGGAATTTGGCTCC |
| | | ACCGCTGACGTGAAGCCGGAGGAAGTCGAGTGGCAGACCGCGGC |
| | | GATCGAGGGCAAACTTGATCTACTGGTGACCCTCGATTTCCGCAT |
| | | GTCCAGTACCTGCCTGTTCTCCGATATCGTCCTGCCGACCGCCACC |
| | | TGGTATGAAAAAGACGATATGAACACCTCAGACATGCACCCGTTT |
| | | ATCCACCCCCTTTCCGCCGCCGTCGACCCGGCCTGGGAGGCGAAA |
| | | AGCGACTGGGAAATCTACAAGGACATCGCCAAAAGTTTCTCTGA |
| | | AGTGTGCGTCGGCCACCTTGATAAAGAGACCGATGTGGTACTGGT |
| | | ACCGCTGCAGCATGACTCTCCGGCGGAGCTGTCACAGCCGTTCGA |
| | | AGTCCTCGACTGGCGCAAGGGCGAATGCGATCTCATTCCAGGCAA |
| | | AACCGCGCCGTCAATTGCGCTGGTTGAACGTGACTACCCGGCCAC |
| | | CTGGGAACGCTTCACCTCGCTGGGGCCGTTACTCGATAAGCTCGG |
| | | CAACGGCGGTAAAGGCATTTCGTGGAATACGCAAAGCGAGGTCG |
| | | ACTTCCTCGGCAAACTTAACTACGTCAAGCCAGACGGTCCGGCCA |
| | | AAGGCCGTCCGCGTATCGACAGCGCTATCGACGCCAGTGAAGTC |
| | | ATCTTAAGCCTGGCGCCGGAAACCAACGGCCAGGTGGCGGTGAA |
| | | AGCCTGGCAAGCGCTGGGAGAATTTACCGGCCGCGACCACACTC |
| | | ATCTGGCGCTGAATAAAGAGGATGAGAAAATCCGCTTCCGCGAT |
| | | ATTCAGGCGCAGCCGCGCAAGATCATCTCCAGCCCAACGTGGTCT |
| | | GGTCTGGAAAGCGAACATGTCTCCTATAATGCCGGTTATACCAAC |
| | | GTTCACGAACTGATCCCGTGGCGTACCCTCTCCGGGCGGCAACAG |
| | | CTGTATCAGGATCATCCCTGGATGCGCGCCTTCGGCGAGAGTCTC |
| | | GTGGCCTATCGCCCACCGATCGATACGCGCAGCGTCAGCCAGATG |
| | | AAGGAAGTGCCGCCAAACGGCTTCCCAGAAAAAGCGCTGAACTT |
| | | CCTGACCCCGCATCAGAAATGGGGTATCCACTCGACCTATAGCGA |
| | | AAACCTGCTGATGCTGACGTTGTCGCGCGGCGGGCCCATCGTCTG |
| | | GCTAAGCGAAACCGACGCAAAAGAACTGGGGATTGAAGATAACG |
| | | ACTGGATCGAAGCCTTCAACGCCAACGGCGCCCTCACCGCGCGTG |
| | | CGGTGGTGAGCCAGCGCGTGCCGCCGGGCATGACCATGATGTATC |
| | | ACGCTCAGGAACGCATTATGAACATTCCAGGTTCGGAGGTGACCG |
| | | GTCGCCGGGGCGGCATCCACAACTCCGTGACCCGCGTCTGTCCTA |
| | | AACCCACGCATATGATTGGCGGCTATGCCCAACTGGCTTACGGCT |
| | | TTAACTACTACGGCACCGTCGGCTCCAACCGCGACGAGTTCATTA |
| | | TGATCCGCAAAATGAAAAATATTGACTGGCTGGATGGCGAAGGT |
| | | CGTGACCAGGTACAGGAGGCGAAGAAATGA |
| pyrroloquinoline | pqqF | >ADJCKNHF_01048 hypothetical protein |
| quinone | | ATGACCACCGCCACCCGCATCGTGGAACTGGCAGGCGGGATTCG |
| biosynthesis | | CGCAACGCTGGTTCATCAGCCGCAGGCGACGCGTGCTGCGGCGTT |
| protein PqqF | | GGTTAAGGTGGGTGCCGGAAGCCATCACGAAACCGATGCCCTGC |
| | | CGGGGCTGGCCCATTTACTGGAACATTTGCTGTTTCGCGGCAGCC |
| | | AGCGCTATCACGCAGACGAGCGATTAATGGCGTGGATCCAACGC |
| | | CAGGGCGGCAGCGTCAACGCCACCACCCTTGCCCGCCACAGCGC |
| | | CTATTTCTTCGAGGTCGCCGCCAGTAGTCTGGCCGAGGGCATCAC |
| | | CCGTCTACGGGATACGCTCCAGGCGCCGCTGCTGTCGCCTGAAGA |
| | | TATCCGTCAGGAACTGGCGGTCATCGATGCGGAAAACCGGCTTAT |
| | | CCAGCAGCACGATCCCTCCCGCCGTGAAGCCGCAGCCCGTCACGC |
| | | GATGGCGGCGCCCGCGGCTTTTCGCCGTTTTCAGGTCGGCAGCAT |
| | | GGATTCTCTGGGCGGAAACTTGCCTGCGCTGCAGGTTGCGCTCGG |
| | | CGAGTTTCATCAGCGCTATTATGTCGCCAGCCATCTGCAGCTATG |
| | | GCTTCAGGGGCCGCAGTCGCTTGATGAATTAGCCGCATTGGCCCA |
| | | TTTTTTTGCCGCGGGATTCCCTGGCGGCCTGCCGCCGGAAAGCCC |
| | | GCCGTCTGCGTATTTTAGCAACAATGTGGATCTTCAACTGGCCGT |
| | | TGAAGACCAACCGGCGGTATGGCGTTGTCCGCTCATTGCAGTAAG |
| | | TGACAACGTCACTACTTTTCGTGAATTCTTACTGGATGAAGCGCC |
| | | CGGCAGCCTGCTGGCCGGGCTACGTGAATGCGGGCTCGCGGACG |
| | | ATGTCGCGCTGAACTGGCTTTACCAGGATGAACGCGTCGGCTGGC |
| | | TGGCGCTGGTATTTACCAGCGACCATCCCGACGCCATCCAGCAGC |
| | | ATCTCGAGCGATGGCTGCAGGCATTGCGCCACACCTCGCCTGAAC |
| | | AGCAGTTGCACTATTACAGGCTGGCGCAGAGGCGTTTTAACGCAC |
| | | TCACCCCGCTCGAACAGTTACGTCAGCGGGCATTGGGTTTTGCGC |
| | | CCGATTCCCCGCCCATCGACTTTGCCCGTTTCTGCGCCAGCTTGCT |
| | | GACGGCGCCAGCCTCTTCGCTGGTGTGCCGCAAAATAGCGGCTGG |
| | | CGATGGCGTGGCAACCCAGGGCTTTACCTTGCCGCTCGGCCGCAG |
| | | GCAACCACGGACAGTCGTGATTGATCCGCTGGCATTTAGCTTCTA |
| | | TCCGCAGGCGGCGACCACGGCAGCGCCTGACCTGCCGCCAACGA |
| | | CCAGCCCGCTGCTGCATGTCATTGCAGATAATCAAACGCCAACGC |
| | | TGATCGTTCGCAAGCCATTCTATAGCGTGCTTAGTCAGGCGCAGG |
| | | GGATGGCGATAAGCCAGCAGCTCCGTCCCTTGCTCGCGCAACTTC |
| | | GCCATGCTGGCGGCAGCGGCGAGTGGCAAACGGTGGACGGCAAC |
| | | TGGCAGCTTACCCTACAGCTACCGGAATCCGGCGGCGTAGCGGA |
| | | GCGCGTCGTCACCGCACTTATGCGCCGATTAGCCCAACCCGCTCC |
| | | GGGGACGACGGTCGCACCTGAAACCATTGCGATTCGCCAGCTACT |
| | | GCAACAATTACCTGAACATCTGACTATCGCCCGGGCGCAAGAGG |
| | | GTTGGCTGGCGGCGATGGTGGGCGGCAGCGGCAATCTGGCGCAG |
| | | CAGGTTGCCCGTCAGTTGACGCTGCTCAATACCCCGATCAACGCC |
| | | GAGCTTCACTCTTCGGCGGGCTGTCGCCGCGGTATTCAGCGTATT |
| | | CCCCACGCCAGCTCGGATAACGCGCTATTAGTTTTTATTCCATTGC |
| | | CCGAGGGGGCATCGCTGGCCGCGCTGCAACTGCTGGCGCTACTTT |
| | | GCGAGCCGCAATTCTTCCAGCGTCTGCGGGTCGAACAGCAGATTG |
| | | GCTACGTGGTGAGCTGCCGTTATCAGCGGATCGCCGATCGCGATG |
| | | GCCTGCTGCTGGCGCTGCAGTCGCCAGATCGTTCCATCAGTAACC |
| | | TGCTACGCTGCTGTAAAACCTTTCTCCGCCAGTTAACGCTGGGTG |
| | | GGCAAGCTGAGTTCAGTCAGTTACAACACCAGTTGGCTGAGCAGC |
| | | ACGGTCTGCCGCAGGACGCCAGCGCCACCGCACTCTCCGCCCTGC |
| | | ACCAGCGCTATCATCTACCGGTAGCCACGCCGCAGAACGTTAACG |
| | | ACCTACAGCTGGACGAGGTCATCGCGCTATGGCGTGAGATAACTC |
| | | GCCGTCGCCGCAGCTGGCGAATCCTGTATAGCGCGCCGACGACGT |
| | | CCGCCACTTAA |
| PqqA peptide | pqqA | >ADJCKNHF_01049 PqqA peptide cyclase |
| cyclase | | GTGAGCCAGAATAAACCCGCCGTTAATCCGCCGCTGTGGCTACTG |
| [EC:1.21.98.4 | | GCGGAGCTGACCTATCGCTGTCCGTTGCAGTGCCCCTACTGCTCT |
| | | AACCCGCTGGACTTTGCTCAGCAGGAGAAAGAACTCACCACCGA |
| | | ACAGTGGATTGAGGTCTTTCGCCAGGCGCGGGCCATGGGTAGCGT |
| | | GCAGATGGGTTTCTCCGGCGGCGAACCGCTGACGCGCAAAGATCT |
| | | GCCGGAGCTTATCCGCGCCGCGCGCGACCTCGGTTTTTACACCAA |
| | | TCTGATTACGTCAGGCATTGGGCTCACCGAGAGCAAACTCGACGC |
| | | CTTCAGCGAGGCCGGGCTGGATCATATCCAGATTAGCTTCCAGGC |
| | | CAGCGATGAAGTGCTTAACGCGGCGCTGGCGGGCAACAAAAAAG |
| | | CGTTCCAGCAGAAGCTGGCGATGGCGAAAGCGGTAAAAGCACGC |
| | | GACTACCCGATGGTGCTGAACTTTGTCCTCCATCGCCACAATATC |
| | | GACCAGATCGATAAAATCATCGAGCTGTGTATCGAGCTGGAAGC |
| | | GGATGATGTTGAGCTCGCCACCTGCCAGTTTTACGGCTGGGCGTT |
| | | CCTCAATCGTCAGGGGTTACTGCCGACCCGCGAACAGATCGCCCG |
| | | AGCTGAACAGGTCGTTGCTGATTACCGCCATAAAATGGCGGCTAA |
| | | CGGTAATCTGACCAATCTGCTCTTTGTCACCCCGGACTACTACGA |
| | | AGAACGCCCTAAAGGCTGTATGGGCGGCTGGGGCTCGATATTCCT |
| | | CAGCGTGACCCCGGAAGGCACCGCGCTGCCGTGTCACAGCGCGC |
| | | GCCAGTTACCGGTCGAGTTCCCATCGGTGCTGGAGCAGAGCCTGG |
| | | AATCGATCTGGTATGACTCGTTTGGCTTCAACCGCTACCGCGGCT |
| | | TCGACTGGATGCCGGAGCCGTGCCGTTCCTGTGATGAAAAAGAG |
| | | AAAGATTTCGGCGGCTGCCGCTGTCAGGCCTTTATGCTGACCGGC |
| | | AATGCCGATAACGCTGACCCGGTGTGCAGCAAATCGCCGCACCA |
| | | CCATAAAATCCTTGAAGCGCGGCGCGAAGCGGCCTGTAGCGACA |
| | | TCAAAATCAGCCAGCTGCAGTTCCGTAACCGCACCCGTTCACAGC |
| | | TGATCTATAAAACCCGGGAATTGTGA |
| pyrroloquinoline | pqqD | >ADJCKNHF_01050 PqqA binding protein |
| quinone | | ATGCAAAAAAGCGCAATCATCGCCTTTCGTCGCGGCTACCGCCTG |
| biosynthesis | | CAGTGGGAAGCTGCTCAGGATAGCCACGTGATCCTCTATCCGGAA |
| protein D | | GGAATGGCGAAACTAAATGAGACCGCCGCCGCGATCCTCGAACT |
| | | GGTCGATGGTCAACGCGACGCGGCAAAAATAATCGCCGAACTCA |
| | | ACGCCCGTTTCCCGGAAGCCGGCGGCGTCGATGACGACGTCATCG |
| | | AATTCCTGCAGATAGCTTACCAACAGAAGTGGATTATCTTCCGTG |
| | | AGCCAGAATAA |
| pyrroloquinoline- | pqqC | >ADJCKNHF_01051 Pyrroloquinoline-quinone synthase |
| quinone | | ATGCAGATTCGCGAAACCCTATCGCCACAGGCGTTTGAACAAGCG |
| synthase | | CTACGGGAAAAAGGCGCCTACTACCATATCCATCATCCGTACCAT |
| [EC:1.3.3.11] | | ATTGCGATGCATAACGGCGAGGCCAGCCGCGAACAAATCCAGGG |
| | | CTGGGTGGCGAACCGTTTTTACTACCAGACCAATATCCCGCTAAA |
| | | AGACGCGGCGATTATGGCAAACTGCCCCGATCCACACACCCGAC |
| | | GCAAATGGGTGCAGCGGATCCTCGACCACGATGGCAGTAACGGC |
| | | CAGGAAGGCGGTATTGAAGCCTGGCTGCAGTTGGGTGAAGCCGT |
| | | GGGGCTAAGCCGTGATGAGTTACTCAGCGAGCGCCACGTGCTGCC |
| | | CGGGGTGCGTTTTGCCGTCGACGCCTACGTTAATTTTGCCCGGCG |
| | | GGCCAACTGGCAGGAAGCGGCGTGCAGTTCGCTGACCGAACTGT |
| | | TTGCCCCGCAAATCCATCAGTCGCGCCTCGACAGCTGGCCGCAGC |
| | | ACTACCCATGGATCAAAGAAGAAGGCTATTTCTACTTCCGCAGCC |
| | | GCTTAAGCCAGGCCAATCGCGACGTCGAACATGGGCTGGAGCTG |
| | | GCGAAGATCTACTGCGATAGCGCGGAAAAGCAGAACCGGATGCT |
| | | GGAGATCCTGCAGTTTAAGCTCGACATTCTGTGGTCGATGCTGGA |
| | | TGCCATGACCATGGCCTATGCGTTGCAGCGCCCGCCCTATCATAC |
| | | GGTCACCGACAAGGCGGCCTGGCACACGACCCGACTGGTATAA |
| pyrroloquinoline | pqqB | >ADJCKNHF_01052 Coenzyme PQQ synthesis protein B |
| quinone | | ATGTTTATAAAAGTCCTCGGTTCCGCCGCTGGCGGAGGTTTTCCG |
| biosynthesis | | CAGTGGAATTGTAACTGCGCCAACTGTCAGGGGCTGCGCAATGGC |
| protein B | | ACAATTCAGGCCACCGCCCGCACCCAGTCTTCGATTATCGTTAGC |
| | | GATAACGGCAAAGAGTGGGTGCTGTGTAACGCCTCTCCGGATATC |
| | | AGCCAGCAAATTGCCCACACGCCAGAGTTAAACAAACAAGGCGT |
| | | GCTGCGCGGCACGCATATTGGCGGCATTATCCTTACCGATAGCCA |
| | | GATTGACCATACTACCGGGCTGCTGAGCCTACGCGAAGGGTGTCC |
| | | CCATCAGGTCTGGTGCACGCCGGAGGTACATGAGGATCTCAGCAC |
| | | GGGTTTTCCCATTTTTACTATGTTACGGCACTGGAACGGCGGCCT |
| | | GATTCACCATCCTGTCACCCCGCTGAATAGTTTTACCGTTGACGCC |
| | | TGTCCCGACCTGCAGTTTACCGCCGTCCCCATCGCCAGCAATGCG |
| | | CCGCCCTACTCGCCGTTTCGCGATAGGCCGCTGCCGGGCCATAAC |
| | | GTGGCGCTGTTTATCGAAAACCGCCGCAACGGCCAGACGCTATTC |
| | | TATGCGCCGGGGCTTGGCGAACCGGACGAAGCGCTCTTGCCGTGG |
| | | CTGAAAAAAGCGGACTGTCTGCTGATTGACGGCACCGTATGGCA |
| | | GGATAATGAACTGCAGGCTGCCGGCGTCGGGCGCAATACCGGTC |
| | | GTGATATGGGCCATCTGGCGCTTGGCGATGAACACGGCATGATGG |
| | | CGCTGCTGGCCTCACTCCCGGCGAAGCGCAAGATTCTGATTCATA |
| | | TCAACAATACTAATCCAATCCTTAACGAGCAGTCGCCGCAGCGTA |
| | | ACGCCCTGACGCAACAGGGAATTGAAGTGAGCTGGGACGGAATG |
| | | CCTATCACGCTTCAGGACTAA |
| pfam13353 | pfam13353 | >ADJCKNHF_01563 Anaerobic sulfatase-maturating enzyme |
| | | ATGAAAAAAAGCACCACCATTCCATTAACGCCATTACAGAACCA |
| | | GGGGCGCTATCTGCCCGCCTATAAACGCCGCTATCACATGATGGC |
| | | GAAACCCAGCGGCTCCACCTGCAACCTGGACTGTCAATACTGTTT |
| | | TTATCTGCATAAAGAGCAGCTTTTACATCAGCCGCACGACAAAGG |
| | | AATGAGCGACGAGGTACTGGAGAATTTCATTCGTCAGTACATCCA |
| | | AAGCCAGGACGGCGAAGAGATTATTTTCTCCTGGCAGGGCGGCG |
| | | AGCCAACCCTGATGGGACTGGAGTTTTTCGAAAAGGTCGTTGAGC |
| | | TGCAAAAAAAATATCAGCCGAAGCACCAGCGCATTGAGAACGAT |
| | | CTGCAGACCAATGGCGTGCTGATTAACGATAAGTGGGCCGCCTTC |
| | | TTGAAGGCGCATAACTTCCTGGTCGGGGTATCGATCGATGGCCCG |
| | | CGCGAGATCCACGATCGCTTCCGCGTGACGCGCAGCGGCAAGCC |
| | | GACCTTCGATAAGGTTATGGAAGGGATCGCGGCGCTGAAGCGCC |
| | | ACGGCGTGCCGTTCAACGCGCTGGCAGTCGTTAACCGGGTGAATG |
| | | CCCGTTTCCCGCGCGAGGTATATCGTTTTCTTACCCGCGAACTCGG |
| | | CGCCACCTATATCCAGTTTACGCCCTGCGTAGAGGCCGCCGAGTT |
| | | TAAAACTACCGCGCCGCAGTTCTGGCGCGAAGAGACGATCCCCAT |
| | | CACCGGCAGCCGCCGGGCGAAACCGGGGGATCTGGATTCAATCG |
| | | TCACCGACTGGTCGGTGGATCCGGACGATTGGGGGCATTTTCTGA |
| | | CCGAAGCTTTTGATGAGTGGGTGACCTGTGATCTGGGCCGCGTGC |
| | | AGGTAAATTTGTTCGAAACCGCCGTGGTGCAGACTATGGGGCTTC |
| | | CATCCCAGTTATGCATCACCGCGCCGTTTTGCGGCAAGGCGCTGG |
| | | CGATTGAGAAAAACGGCGACGTCTACTCCTGCGATCACTACGTCT |
| | | ACCCGGAATATAAGCTTGGCAATATTAAGGCGCATAAGCTGGCG |
| | | CATATGGTCTTTTCCGAGCGGCAGAAAGTGTTTGGCATGGGCAAG |
| | | AAAGAGACGCTGCCCGCCTACTGCAAAAGCTGCCCGCATCTGAAT |
| | | TTATGCTGGGGCGAATGCCCGAAAAACCGCATCGTGCGCGCCCCG |
| | | GACGGCGAAGAAGGGCTCAATTATTTGTGTCCCGGCTTTCGCCAT |
| | | TTTTACGCGACGGTTAAACCGACGCTTGAGAAAATTGCCGCCATG |
| | | CTGAAGTAG |
| pfam13186 | pfam13186 | >ADJCKNHF_01571 Anaerobic sulfatase-maturating enzyme |
| | | ATGAAACACAGCACCACCGTGCCTGTTAACGCATTACCTTCGGCA |
| | | GAAAAGTACGCGGGTAACGGCCCCGCCTACCAACGTCGCTTTCAT |
| | | GTGATGGCGAAACCCAGCGGCTCCGCCTGTAATCTCGATTGTAGC |
| | | TACTGCTTTTACCTGCACAAAGAACACTTACTCCAGCAGGAAAAG |
| | | CGCAGCTACATGAGCGATGAAACGCTGGAAAACTTTATCCGCCA |
| | | GTACATCGATGGACAGGATGGCGAACAGGTGGTCTTCTCCTGGCA |
| | | GGGCGGCGAACCGACCCTAATGGGACTGGAGTTTTTCCACAAAGT |
| | | GGTGAAATTCCAACAGCAATATAAAAAGCCCGGCCAGCGGATCG |
| | | AAAACGATCTGCAAACCAACGGTATCCTGATTAACGATGCCTGGG |
| | | CTGAATTCCTCAAAACGAATCATTTCCTTGTCGGCCTGTCGATTGA |
| | | CGGCCCGCGAGAGTTGCACGACCGTTATCGCATCACCCGCAGCGG |
| | | CAAACCGACATTTGATAAAGTGATGGCCGGCGTCGACGCCCTGA |
| | | AGCGCCACGGCGTTCCATTCAATGCGCTGGTGACCATCAACCGTA |
| | | CCAACGCCCGTTTCCCATTAGAGGTTTACCGCTTTGTTACCCGCGA |
| | | ACTGGGGGCGACCTATGTGCAGTTTAACCCCTGCGTCGAACCGGT |
| | | GGACTTTACCCAGACCGCGCCCCATTTCTGGCGTGATGACACTAT |
| | | CCCCACCGTCGGCAGCCGCCGCGCGCGCCCCGGCGATTTAGACTC |
| | | CATCGTCACCGACTGGTCGGTCGACCCGGACGACTGGGGACGCTT |
| | | CCTGATAGCCACCTTCGAAGAGTGGGTGAACAACGATCTTGGCCG |
| | | CGTACAGGTCAATCTGTTCGAAACCGCCGTCGCCCAGATGATGGG |
| | | TCTGCCGGCGCAAATTTGCACCACCGCGGAGTTTTGCGGCAAAGG |
| | | ACTCGCGGTCGAGAAAAACGGCGATGTTTTCTCCTGCGATCACTA |
| | | CGTCTATCCGGAGTATCAAATCGGCAATATCGCCGATAACAGCCT |
| | | GGCGCGAATGGCTTTCTCCGAACGTCAGCAGGCTTTCGGTATGGG |
| | | TAAATGCGACACGCTGCCCCAGCAGTGCAAGCAGTGCCCTTACCT |
| | | GAAGCTTTGCCACGGCGAGTGCCCGAAAAACCGTCTGGTGCTCAC |
| | | TACCGACGGCGAAGCCGGTTTGAACTATCTCTGCCCGGGCATTAA |
| | | AGCCTTTTTCAACTATGCCGAGCCGATTCTGGCGGGCATCGTCAC |
| | | GCTGGTGAAACGCGATTTTAAAGGAGTTAGCCGATGA |
| agmatinase | speB | >ADJCKNHF_01700 Agmatinase |
| [EC:3.5.3.11] | | ATGAGCACTTTAGGTCATCAATACGATAACTCCCTGGTATCCAAC |
| | | GCCTTTGGTTTCCTGCGTCTGCCGATGAACTTTATGCCGTATGAAA |
| | | GCGATGCGGATTGGGTCATCACCGGCGTGCCGTTTGATATGGCGA |
| | | CCTCCGGGCGCGCGGGCGGCCGTCATGGCCCGGCAGCGATCCGTC |
| | | AGGTGTCAACCAACCTCGCCTGGGAGCACAACCGTTTCCCATGGA |
| | | ACTTCGACATGCGCGAACGCCTGAACGTCGTGGACTGCGGCGATC |
| | | TGGTCTACGCCTTCGGCGACGCCCGCGAAATGAGCGAGAAACTG |
| | | CAGGCGCACGCGGAGAAACTGCTGGCGGCCGGTAAACGCATGCT |
| | | CTCCTTCGGCGGCGACCATTTCGTCACCCTGCCGCTGCTGCGCGC |
| | | CCACGCCAAGCATTTCGGTAAAATGGCGCTGGTACACTTCGATGC |
| | | CCACACCGATACCTACGCCAACGGCTGTGAATTCGACCACGGCAC |
| | | CATGTTCTACACCGCGCCGAACGAAGGGCTTATCGATCCGAACCA |
| | | CTCCGTGCAGATCGGTATTCGTACCGAATTCGATAAAGATAACGG |
| | | TTTTACCGTGCTTGATGCGGGCCAGGTTAACGACCGCAGCGTCGA |
| | | TGACATCATCGCTCAGGTGAAGCAGATCGTCGGCGATATGCCGGT |
| | | GTACCTGACCTTCGATATCGACTGCCTGGATCCGGCATTTGCGCC |
| | | AGGTACTGGTACGCCGGTGATCGGTGGACTCACCTCCGACCGTGC |
| | | GATCAAACTGGTGCGCGGCCTGAAGGATCTGAACATCGTCGGGA |
| | | TGGACGTGGTTGAAGTCGCCCCGGCCTATGACCAGTCCGAAATCA |
| | | CCGCCCTGGCGGCGGCGACTCTGGCGCTGGAGATGCTTTATATTC |
| | | AGGCGGCGAAGAAAGGCGACTAA |
| pyrroloquinoline | pqqF | >ADJCKNHF_01868 Protease 3 |
| quinone | | ATGCCCCGCAGTTTATGGTTCAAAGTTCTTGTTGTAGTGGTCGCCC |
| biosynthesis | | TTTGGGCGCCGTTAAGTCAGGCAGACACCGGATGGCAACCGATTC |
| protein PqqF | | AGGAAACCATCCGTAAAAGCGAAAAAGATCCCCGTCACTATCAG |
| | | GCTATTCGCCTGCAAAACGGCATGGTCGTGCTGCTGGTATCCGAT |
| | | CCGCAGGCGGTAAAATCGCTGTCGGCGCTGGTGGTGCCGGTGGGT |
| | | TCATTACAGGATCCGGCCGACCATCAAGGGCTGGCGCACTATCTC |
| | | GAGCATATGACCTTAATGGGCTCGCAGAAGTACCCGCAGCCTGAC |
| | | AGTCTTGCTGAATTCCTCAAAATGCACGGCGGCAGTCATAATGCC |
| | | AGCACGGCGCCGTATCGCACCGCGTTCTACCTTGAAGTGGAAAAC |
| | | GATGCTCTCAACGGCGCTGTCGATCGGCTGGCCGATGCCATTGCC |
| | | GCGCCGCTGCTGGATAAAAAATATGCCGAACGCGAACGTAACGC |
| | | GGTCAACGCCGAATTGACTATGGCGCGTACCCGGGACGGGATGC |
| | | GCATGGCGCAGGTGAGCGCCGAAACCATCAACCCTGCGCATCCG |
| | | GCTTCACAATTCTCCGGCGGTAACCTCGATACACTGAGCGATAAG |
| | | CCTGGCAGCCCGGTGCTTGATGCGCTACACGCTTTCCGCGACCGC |
| | | TGGTACTCGGCAAACCTGATGAAGGCAGTTATCTACAGCAATAAG |
| | | CCGCTGCCTGAGTTGGCAAGCATTGCCGCCGCGACCTACGGGCGG |
| | | GTGCCTAATCATGACATCAGCAAGCCGGAGATTACCGTACCGGTC |
| | | GTGACCGATGCGCAGAAGGGCATTGTGATTCACTACGTGCCGGCG |
| | | ATGCCGCGTAAGGTGCTGCGCGTTGAATTCCGCATCGATAACAAC |
| | | AGCGCTCAGTTCCGCAGCAAGACCGATGAGCTGGTGACCTATATG |
| | | ATTGGCAACCGCAGCCCCGGCACTTTATCCGACTGGCTGCAGAAG |
| | | CAGGGGCTGGCGGAAGGGATTCGCGCGGATTCCGATCCGGCGGT |
| | | CAACGGTAATAGCGGCGTGCTGGCGATCTCCGCTACCCTCACCGA |
| | | TAAAGGCCTGGCGCACCGCGATGAAGTTACCGCCGCTATTTTCAG |
| | | CTACCTGAATTTGCTGCGTACACAGGGTATTGATAAGCGCTACTT |
| | | TGATGAATTAGCCCACGTGCTGGAGCTCGATTTCCGTTATCCGTC |
| | | GATTAACCGCGATATGGATTATGTGGAGTGGTTGGCCGATACCAT |
| | | GATTCGCGTGCCGGTAGAGCACGTACTGGACGTGGTTAATATCGC |
| | | CGACCGCTATGATGCCCAGGCGATCAAAGACCGTCTGGCGATGAT |
| | | GACCCCGCAAAATGCGCGTATCTGGTACATCAGCCCGAATGAGCC |
| | | GCACAACAAAACCGCTTATTTCGTCAACGCGCCGTACCAGGTCGA |
| | | TAAAATTAGCGCCCGGACCTTCACCGACTGGCAGCAAAAATCTGC |
| | | GGCCATCAAGCTGCAGCTGCCGACGCTGAACCCGTATATTCCGGA |
| | | TGATTTCACGCTGATTAAGAGCGATAAGGCCTACCCGCATCCGGA |
| | | GCTTATCGTCGATGAGCCGACGCTGCGCGTGGTGTACGCGCCAAG |
| | | CCAGTACTTTGCCAGTGAACCGAAGGCCGACGTCTCGCTGGTGCT |
| | | ACGTAACCCGCAGGCGATGGACAGCGCGCGCCGTCAGGTGATGT |
| | | TCGCGTTGAATGATTACCTGGCCGGTATCGCCCTCGATCAGCTTA |
| | | GCAATCAGGCCGCGGTAGGCGGGATAAGCTTCTCGACCGGCGCC |
| | | AACAACGGTCTGATGGTTAATGCCAACGGCTATACCCAGCGTCTG |
| | | CCGCAGCTCTTTACCGCGCTGCTGGACGGCTACTTTAGCTATACG |
| | | CCGACCGAAGAGCAGCTTGAGCAAGCGAAGTCCTGGTATGCCCA |
| | | GATGATGGATTCGGCGGACAAAGGCAAAGCCTATGACCAGGCGA |
| | | TTATGCCGATTCAGATGCTGTCGCAGGTACCCTATTTTGAGCGTA |
| | | AAACACGTCGCGATTTACTACCGTCAATCACTCTGAAAGAGGTGA |
| | | TCAACTACCGCGATAACCTGAAAGCAAGAGGGCGGCCGGAGCTG |
| | | CTGGTCATCGGTAACATGAGCGCCAGACAGTCCACCGATCTAGCC |
| | | CGCCAGATCCAAAAACAGCTTGGCGCCGATGGCAACGAGTGGTG |
| | | TCGCAACAAAGACGTGCTGATCGACAGCAAACAGCTGGCGATGT |
| | | TTGAGAAAGCAGGCAGCAGCACCGACTCCGCGCTGGCGGCGGTT |
| | | TTCGCGCCGCCAAATGTCGATGAATACAGCAGCATGGCCGCCAGT |
| | | TCGCTGTTAGGGCAGATTATTCAGCCCTGGTTCTACAACCAGTTA |
| | | CGTACTGAAGAACAACTCGGCTATGCGGTATTCGCCTTCTCAATG |
| | | AATGTGGGCCGCCAGTGGGGGATGGGCTTCCTGCTACAGAGCAG |
| | | CGATAAACAGCCGGCCTTCCTCTGGCAGCGCTTCCAGGCCTTCTT |
| | | CCCGACGGCGGAAGCCAAACTGCGGGCGATGAAACCGGAAGAGT |
| | | TTGCCCAGATCCAGCAGGCGGCGATTAGCCAGATGCTGCAGGCG |
| | | CCGCAAACGTTGAGCGAAGAAGCATCAAAGCTGAGCAAAGATTT |
| | | CGATCGCGGTAATATGCGCTTCGATTCACGTGATAAAGTAGTGGC |
| | | TCAGATGAAACTGCTGACGCCGCAAAAACTTGCCGACTTCTTCCA |
| | | TCAGACGGTGGTGGATCCGCAAGGCATGGCACTGTTATCCCAGGT |
| | | TTCCGGCAGCCAGAACGGCAAGACGGAATACGCCGCCCCTGAAG |
| | | GCGGTAAGGTATGGGAAAGCGTCAGCGCATTGCAAAAATCCTTA |
| | | CCCCTGATGCGAGAGAATGAATGA |
| alkaline | phoA, phoB | >ADJCKNHF_02213 Alkaline phosphatase |
| phosphatase | | GTGAAATTATCTGCCCTCTTTATTGCCCTGTTACCGCTGCTGGCTC |
| [EC:3.1.3.1] | | CCCCGGTTATTCATGCGCAAACCACTTCTTCGCCGGTGCTGGAAA |
| | | ATCGCGCGGCGCAGGGCGATATCACGACCCCAGGCGGGGCGCGC |
| | | CGTTTAACGGGCGATCAGACCGAAGCGCTGCGCGCTTCGTTAATC |
| | | AATAAGCCGGCGAAAAATATTATTTTGCTCATTGGCGATGGCATG |
| | | GGTGATTCAGAAATTACCGCCGCACGAAATTATGCCGAAGGCGC |
| | | CGGCGGTTTCTTTAAAGGTATTGATGCCCTGCCGCTGACGGGCCA |
| | | ATACACCCATTATTCTCTGGATAAAAAAACCGGCAAGCCGGATTA |
| | | CGTGACGGATTCCGCCGCGTCGGCAACCGCGTGGACCACCGGCGT |
| | | GAAAAGCTATAACGGCGCGCTGGGCGTTGATATCCACGAAAAAG |
| | | ATCATCAGACCATCCTTGAGCTGGCGAAAGCCGCGGGTCTTGCCA |
| | | CCGGCAATGTCTCGACGGCTGAACTGCAGGATGCGACGCCGGCG |
| | | GCGCAGGTGGCGCATGTGACCTCGCGTAAATGCTATGGCCCTGGC |
| | | GTGACCAGCGAAAAATGCGCCAGCAACGCGCTGGAAAAAGGCGG |
| | | CAAGGGCTCTATCACCGAACAACTGCTGAATGCCCGTCCGGATGT |
| | | CTCTTTAGGCGGCGGGGCGAAGACCTTTGCTGAAACGGCGACCGC |
| | | AGGGGAGTGGCAGGGGAAAACCCTGCATGAGCAGGCGGTCGCTC |
| | | GCGGCTATCAGATTGTGACCGACGCCGCTTCACTCGCCGCTATCA |
| | | GCGAAGCAAATCAGGGTAAACCGCTGCTCGGTCTGTTCTCCGACG |
| | | GCAATATGCCGGTGCGGTGGGAAGGCCCGAAAGCCTCTTATCAC |
| | | GGCAATATCGACAAACCGCCGGTAACCTGTACGCCAAACCCGAA |
| | | ACGCGATGCTTCCGTACCGACGCTGGCGCAGATGACCGAAAAAG |
| | | CGATTGATCTCCTGAGCCGCAACGAGAAAGGTTTCTTCCTGCAGG |
| | | TTGAGGGGGCGTCTATCGATAAACAGGACCACGCGGCAAACCCT |
| | | TGCGGCCAGATTGGCGAAACCGTCGACCTCGATGAAGCGGTACA |
| | | GAAAGCGCTGGAATTTGCCAAAAAAGAGGGCAATACCCTGGTGA |
| | | TCGTCACCGCCGATCACGCCCACTCCAGCCAGATTATCCCGGCAG |
| | | ACACGAAAGCGCCGGGCCTGACCCAGGCGCTGACGACCAAAGAT |
| | | GGCGCGGTAATGGTGATGAGCTATGGCAACTCTGAAGAAGAGTC |
| | | GATGGAGCACACCGGCACCCAACTGCGTATTGCCGCGTACGGAC |
| | | CGCATGCGGGCAACGTGGTGGGTCTCACCGACCAGACCGATCTGT |
| | | TTTACACCATGAAAGACGCGCTGGGCCTGAAATAA |
| | | >ADJCKNHF_02227 Phosphate regulon sensor protein PhoR |
| two- | phoR | GTGCTGGAACGGCTGTCATGGAAAAGGCTGGCGCTTGAGCTTTTC |
| component | | TTATGTTGTATCCCGGCCCTGATCCTCGGGGCGTTTTTCGGTCATC |
| system, | | TGCCGTGGTTTTTACTGGCGGCGGTGACCGGATTGCTTATTTGGC |
| OmpR | | ATTTCTGGAATTTGTTACGTCTGTCGTGGTGGCTGTGGGTCGATCG |
| family, | | CAGTATGACGCCGCCGCCGGGAAGCGGCAGTTGGGAACCGCTGC |
| phosphate | | TGTATGGTCTGCATCAAATGCAGATGCGTAATAAAAAACGTCGCC |
| regulon | | GCGAGCTGGGGAGCCTCATCAAGCGCTTTCGCAGCGGGGCGGAA |
| sensor | | TCCTTACCCGATGCGGTGGTGCTGACCACCGAAGAGGGCGCCATC |
| histidine | | TTCTGGTGTAACGGCCTGGCGCAACAAATCCTTAATCTACGCTGG |
| kinase PhoR | | CCGGACGATAGCGGGCAGAATATCCTCAACCTGCTGCGCTACCCA |
| [EC:2.7.13.3] | | GAGTTTGCTAACTATCTGAAAAACCGTGATTTCACCAAGCCCCTT |
| | | AATCTGGTGCTCAATAACGCCCGTCATCTGGAAATTCGCGTGATG |
| | | CCTTATAGCGATAAACAGTGGTTGATGGTGGCGCGCGATGTCACG |
| | | CAAATGCACCAGTTGGAAGGCGCGCGGCGCAACTTTTTCGCCAAC |
| | | GTCAGCCACGAGCTGCGCACGCCGCTCACCGTACTGCAGGGTTAT |
| | | CTCGAGATGATGCAGGAGCAGGTGCTGGAAGGGCCGACGCGGGA |
| | | AAAAGCGCTGCATACCATGCGTGAGCAAACGCAGCGGATGGAAG |
| | | GGTTGGTGAAGCAGTTGCTGACGCTCTCGCGTATTGAAGCCGCGC |
| | | CGGTGCTGGCGATGAATGATAAAATCGACGTCCCGATGATGCTGC |
| | | GGGTGGTTGAGCGCGAGGCGCAAACCCTCAGCCAGGGTAAGCAT |
| | | CAGCTGCACTTTAGCGTCGATGAAACGCTCCAGGTGATGGGCAAC |
| | | GAAGAGCAGTTGCGCAGCGCGATTTCCAATCTGGTGTACAACGCC |
| | | GTTAACCATACGCCAGCGGGAACTGAGATTCACGTCAGCTGGCA |
| | | GCGGGCGCCACACGGCGCGCTGTTCAGCGTTGAAGACAACGGTC |
| | | CGGGCATTGCGCCTGAACATTTACCCCGGCTAACCGAGCGGTTCT |
| | | ACCGCGTCGACAAGGCGCGCTCCCGGCAAACCGGCGGCAGCGGT |
| | | TTAGGGCTGGCTATCGTGAAACATGCGGTTAGCCATCACGAAAGC |
| | | CGGCTGGAAATCGAAAGTACGGTCGGGAAGGGAACACGCTTTAG |
| | | CTTCCTGCTGCCGGAACGTTTAATTGCCAAAAATGGCGCCTGA |
| Pyoverdine | pvdL | >ADJCKNHF_02461 Enterobactin synthase component F |
| chromophore | | ATGAGTACACGTTTACCGCTGGTTGCGGCGCAGCCGGGAATTTGG |
| precursor | | ATGGCTGAACGCCTCTCCACGCTACCCGGCGCCTGGAGCGTTGCC |
| synthetase | | CACTATGTTGAACTGCGCGGCAATCTTGATCCGGCGCTGCTGAGT |
| PvdL | | AAGGCGATCGTCGCCGGGCTTAAGCAGGCCGATACCCTGAGCAT |
| | | GCGCTTTTGCGAAGATAATGGCGAAGCGTGGCAGTGGGTTGATG |
| | | ACGCGCGCGAGTTTGCCGAGCCGCAAAGCGTTGACCTGCGCCAG |
| | | CAGGCCGATCCGCATATGTCCGCGCTGACGCTGATGCAAAGCGAT |
| | | CTCGGGCAGAATCTGCGCGTCGACAGCGGTAATCCGCTGGTCTGC |
| | | CATCAGCTCATGCGCGTTGGCGACGACTGCTGGTACTGGTATCAG |
| | | CGCTATCACCATTTACTGGTCGATGGTTTTAGTTTCCCGGCCATTA |
| | | CCCGCCAGATCGCCGCTATTTATCGTGCCTGGCAACGCGGCGAAG |
| | | AGACGCCGGATTCACCGTTTACGCCGTTCGCCGAAGTGGTGGAAG |
| | | AGTATCAGCGCTACTACGGCAGCGAGGCGTGGCAGCGCGATAAA |
| | | GCCTTCTGGCAGGCGCAGCGTCAGGCGTTGCCGTCTCCGGCCTCG |
| | | CTATCAGACGCGCCGCTGGCCGGGCGGGCGACCAGCAGCGATAT |
| | | CTGGCGCCTGAAACTGGAGGCCGATCCGGCTGTCTTCAGCCAGCT |
| | | CGCGGCCAGCGCGCCGCAGTGCCAGCGCGCCGATCTGGCGTTGG |
| | | CGCTGACGACGTTGTGGCTGGGGCGGTTGTGCGGCCGAATGGATT |
| | | ACGCGGCTGGCTTTATCTTTATGCGCCGCATGGGATCGGCGGCGC |
| | | TGACCGCCACCGGCCCGGTACTCAACGTACTGCCGCTGGCGGTGA |
| | | ATATCGACGCGCAGGAGACGCTGGCGCAACTGGCGACGCGTCTC |
| | | TCCGGACAACTGAAAAAAATGCGTCGCCATCAGCGCTACGATGC |
| | | CGAGCAAATTGTCCGCGATAGCGGTAAAGCGGCGGGCGACGAAC |
| | | CGCTGTTTGGCCCGGTGCTGAACATCAAAGTCTTTGATTACCAGT |
| | | TGGATATCGACGACGTGCAGGCGCAGACGCATACCCTCGCCACC |
| | | GGCCCGGTGAATGACCTCGAGCTGGCACTGTTCCCGGACGAACGC |
| | | GGCGGTCTGTCGCTGGAGATCCTCGCCAATAAAGCGCGTTATGAT |
| | | GAGGCGACGCTGAACCGCCACGTGTCGCGGCTGACCGCGCTGCT |
| | | GGCGCAGTTCGCCGCCAACCCGGCGCTACGCTGCGGCGAGGCGG |
| | | AGATGCTGTCGGCGGAAGAAGGCGCGCAGCTGGCATTGATCAAC |
| | | AATACCGCGATGCCGCTGCCGACCACTACCCTCAGCGCACTGGTG |
| | | GCGGAACAGGCGCGGAAAACGCCGGATGCCCCGGCGCTGGCGGA |
| | | TGCCAACTGGCGCTTTAGCTACCGCGAAATGCGCCAGCAGGTGGT |
| | | GGCGCTGGCCAACCTGCTGCGGCAACGCGGCGTGAAGCCGGGGG |
| | | ATAGCGTGGCGGTTGCCTTACCGCGCTCGGTGTTCCTGACGCTGG |
| | | CGCTGCACGGCATTGTCGAAGCCGGCGCCGCCTGGCTGCCGCTGG |
| | | ACACCGGCTACCCGGATGACCGTCTGCGGATGATGCTGGAAGAT |
| | | GCGCGGCCATCGCTGCTGATTACCTCTGACGATCAACTGGCCCGT |
| | | TTTAGCGATATTCCGGGACTGACCAGCTTGTGTTATGAGCAGCCG |
| | | CTGGCTGCTGAAGATGATACGCCGCTGGCGCTGTCAAAACCGGA |
| | | ACATACCGCGTATATCATCTTCACCTCCGGTTCCACCGGGCGGCC |
| | | GAAAGGGGTGATGGTCGGGCAGACCGCCATCGTGAACCGCCTGC |
| | | TGTGGATGCAAAATCACTATCCGTTAACCGCCGCCGATGTGGTGG |
| | | CGCAGAAGACGCCGTGCAGCTTTGATGTCTCGGTATGGGAGTTCT |
| | | GGTGGCCGTTTATGACCGGCGCCCAGCTGGTGATGGCGGCGCCGG |
| | | AGGCGCATCGCGATCCACAGGCGATGCAGCGGTTCTTCACGGATT |
| | | ATGGCGTGACCACCACCCACTTTGTTCCGTCGATGCTGGCGGCGT |
| | | TTGTCGCCTCGCTGGATAGCGATAATGTGTCTTCCTGCCGGACGC |
| | | TAAAACGCGTTTTCTGTAGCGGCGAAGCGCTGCCGACGGAACTGT |
| | | GTCGCGAATGGGAGCGCTTAACCGGCGCGCCATTGCATAACCTGT |
| | | ACGGGCCGACGGAAGCGGCGGTGGATGTCAGCTGGTATCCCGCC |
| | | TGCGGGCCAGAGCTGGCGGCGGTAACCGGCAACAGCGTGCCTAT |
| | | CGGCTGGCCGGTATGGAACACCGGATTACGTATTCTTGATGCTGC |
| | | GATGCGCCCTGTGCCGCCGGGCGTGGCGGGTGATTTGTACTTAAC |
| | | GGGCATTCAGCTGGCACAGGGATATATGGGGCGCCCTGATCTCAC |
| | | CGCCAGCCGCTTTATTGCCGACCCGTTTGCCCCCGGCGAGCGCAT |
| | | GTACCGCACCGGCGATGTGGCGCGCTGGCTGGATAACGGGGCGG |
| | | TAGAGTATCTTGGCCGCAGCGACGATCAGTTGAAAATTCGCGGCC |
| | | AGCGCATTGAGCTTGGCGAGATCGACCGGGTGATGGCGGCGCTG |
| | | CCGGACGTCGCGCAGGCGGTGTGCCACGCCTGCGTCTTCAATCAG |
| | | GCGGCGGCCACCGGCGGCGATGCCCGCCAGCTCGTCGGTTATCTG |
| | | GTGAGCGAGTCCGACCAGCCGCTGGACGTGGCGGCGCTGAAGGC |
| | | GCGTCTTAGCGAACAGCTGCCGCCGCACATGGTGCCGGTGGTGTT |
| | | AATCCAGCTGGAATCCTTCCCGCTCAGCGCCAACGGTAAGCTGGA |
| | | TCGTAAAGCGCTACCGCTCCCCTCCTTAAGCAGCGAGCGCAGCGG |
| | | TCGCCCGCCGCAATCGGCTACCGAAATGGCCGTTGCCGCGGCGTT |
| | | CAGCCAGCTACTGGGCTGCGAGGTAAATGACATCGACGCCGATTT |
| | | CTTCGCCCTCGGTGGCCATTCGCTGCTGGCGATGCGGCTGGCGGC |
| | | GCAGCTTAGCCGCGAGCTGGCGCGGCAGGTGACGCCGGGGCAGG |
| | | TGATGGTCGCTTCGACCGTCGGCAAGCTGAGCGCGCTGCTGGCTT |
| | | CCGACCTGAGCGACGAGCAGGCGCAGCGCCTGGGCTTCGATGCG |
| | | CTGTTGCCGCTGCGCGAGAGCGACGGTCCGACGCTGTTCTGCTTC |
| | | CATCCGGCCTCCGGTTTTGCCTGGCAGTTCAGCGTGCTGGCGCGT |
| | | TATCTTAACCCGCGCTGGTCAATTACCGGTATTCAGTCGCCGCGT |
| | | CCGACGGGGCCGATGGCTTCCGCCGCCAACCTCGACGAAGTCTGC |
| | | GAGCACCATCTGCGCACGCTTTTAGCGCAGCAGCCGCATGGGCCT |
| | | TACTATCTGTTTGGTTATTCGCTCGGCGGAACGCTGGCGCAGGGG |
| | | ATTGCCGCCCGTTTACGCCAGCGCGGCGAAGAGGTGGCGTTCCTC |
| | | GGCCTGCTGGATACCTGGCCGCCGGAGACGCAAAACTGGGCGGA |
| | | GAAAGAGGCGAACGGTCTGGATCCCGAAGTGCTGGCGGAAATCG |
| | | CCCGCGAGCGCGAAGCGTTTCTCGCCGCTCAGCAGGGGCAGGCTT |
| | | CCGGCGAGCTGTTCAGCGCGATCGAAGGTAACTACGCCGATGCG |
| | | GTGCGCCTGTTGACGACCGCGCACAGCGCGAAGTTTGACGGCAA |
| | | GGCAACGCTATTTGTGGCGGAGAAAACGCGCCAGAAAGGGATGG |
| | | ATCCGCAGGTCGCATGGGGGCCATGGGTTGGCGAGCTGGAGGTG |
| | | TTCAGCCAGAACTGCGCCCACGTCGAGATTATCTCGCCGCAGGCC |
| | | TTTGAAGCGATAGGCCCGGTAGTGCGGGAGATTCTGGGGTAA |
| salicylate | pchA | >ADJCKNHF_02468 Isochorismate synthase EntC |
| biosynthesis | | ATGGAAACGTCACTGGCTGAGGATGTACAGGAAAAAACGCAGAC |
| isochorismate | | CCTGTCGCCGCAAAGCTTCTTCTTTATGTCGCCGTACCGCAGCTTC |
| synthase | | AGTACCACCGGCTGTTTTAGCCGTTTTTCCCAGCCTGCCGTCGGCG |
| [EC:5.4.4.2] | | GTGATTCGCTGAACGGCGAGTTCCAGCAGAAAATGGCGGCCGCTT |
| | | TTGCCGAAGCCCGTGCGGCGGGGATCCGCAAGCCGGTCATGGTC |
| | | GGGGCGATTCCGTTCGACACCAATCAGCCTTCCGAGCTCTATATT |
| | | CCCGAACGTTGGGAAACCTTCTCCCGTACCGACAAACAGCAGTCC |
| | | GCGCGTTACGCGGCGCCGCTCAAAGCTATGGATGTTGTCGATCGT |
| | | CAGGAGATCCCGGAACAGGACGAGTTTTTGGCGATGGTGGAACG |
| | | CGCCGCCGCGCTGACGGCGACTCCGGAAGTCGACAAAGTGGTGC |
| | | TCTCAAGGCTGATTGATATTACGACCCGCGACCGGGTCGATAGCG |
| | | GCGCGCTGCTGGAGCGGCTGATCGCCCAGAACCCGGCGAGCTTTA |
| | | ACTTCCATGTTCCACTCTCCAATGGTGATGTGCTGCTGGGCGCCA |
| | | GCCCGGAACTGCTGCTGCGTAAAGAAGGGCTGCACTTCAGTTCGC |
| | | TGCCGCTGGCCGGCTCCGCCCGCCGCCAGCCTGACGATGTGCTGG |
| | | ACCGGGAAGCGGGTAACAAGCTACTGGCGTCGGGCAAAGATCGC |
| | | CATGAGCACGAGCTGGTGACGCAGGCGATGAAGAGCGTGCTTGG |
| | | CCCTCGCAGCAGTCATCTGTCGCTACCGGAGTCGCCGCAGCTGAT |
| | | TACCACCCCGACGCTCTGGCATCTGGCGACGCCGATCGAAGGTAC |
| | | GGCGCTGGCGGAAGAAAACGCCATGTCGCTGGCCTGCCTACTGC |
| | | ACCCGACCCCGGCGCTGAGCGGCTTCCCGCACCAGGTGGCGAAG |
| | | CGGCTGATTGCCGAGCTCGAACCGTTCGATCGCCAACTGTTTGGC |
| | | GGCATTGTCGGCTGGTGCGATGACGAAGGCAACGGCGAATGGGT |
| | | GGTGACCATCCGCTGCGCGCGCTTACATCAACGCTCGCTACGCCT |
| | | GTTTGCCGGCGCGGGTATCGTCCCGGCCTCTTCGCCGCTGGGCGA |
| | | ATGGCGTGAAACCGGCGTCAAACTCACCACCATGCTCAACGTATT |
| | | TGGCTTGAATTAA |
| Pyoverdine | pvdL | >ADJCKNHF_02469 Enterobactin synthase component E |
| chromophore | | ATGATTGCATTTACCCGTTGGCCGGAAGAGTTTGCGGCCCGCTAT |
| precursor | | CGTCAAAAAGGCTACTGGCAGGATCTGCCGTTAACCAATCTGATT |
| synthetase | | ACTCGTCATGCGGACAATGACGCCGTGGCGATTATCGACGGCGA |
| PvdL | | GCGGCAGATTAGCTACCGCCAGTTTAACCAACTGGTGGATAACCT |
| | | CGCGTCCTCCCTGCAGCATCAGGGACTCAAGCGCGGAGAAACCG |
| | | CGCTGGTGCAGCTTGGCAACGTCGCCGAGTTCTACATTACTTTCTT |
| | | TGCGCTGCTGCGCATCGGCGTCGCACCGGTTAACGCTTTGTTTAG |
| | | CCATCAGCGCAGCGAACTTAACGCCTATGCCGCGCAGATTAAACC |
| | | GGTGCTGCTGATTGCCGATCGCGAGCACGCGCTGTTTGCTGACGA |
| | | TAGCTTTCTCCATGGTTTTATCGCTGAGCATCCTTCGCTGCGCGTC |
| | | GCGCTGCTGCGTAACGATGGCGGCGAGCGCGACCTGGCAACGGA |
| | | AATTAGCCGCACGGCGGATAACTTTGTCGCCAATCCGACGCCGGC |
| | | TGACGAAGTGGCTTTTTTCCAGCTCTCCGGCGGCAGCACCGGGAC |
| | | GCCGAAGCTTATTCCGCGAACCCATAACGATTACGACTACAGTAT |
| | | CCGCCGCAGCAACGAGATCTGCGGTATTAACGCCGACACGCGCT |
| | | ATCTCAACGCGCTGCCGGCGGCGCACAACTATGCGATGAGCTCGC |
| | | CGGGATCGCTGGGCGTCTTTCTCGCCGGCGGCCGGGTGATCCTCG |
| | | CCGCTGACCCGAACGCCACGTTGTGTTTCCCGCTGATCGAAAAAC |
| | | ATCAGATTAACGTCGCGTCGCTGGTGCCGCCGGCGGTCAGCCTGT |
| | | GGCTGCAGGCTATTCATGAATGGGGCAGCAACGCGCAACTGCAA |
| | | TCGCTGAAGCTGCTGCAGGTTGGCGGAGCGCGCCTGTCCGCGACG |
| | | CTCGCCGCGCGTATCCCGGCGGAAATCGGCTGCCAGCTGCAGCAG |
| | | GTGTTCGGCATGGCGGAAGGGCTGGTTAACTACACCCGCCTCAAC |
| | | GATAGCCCGCAGCGGATTATCAACACCCAGGGTTGCCCGATGTGT |
| | | CCTGACGACGAAGTGTGGGTGGCGGATGCCGACGGTAACCCGCT |
| | | GCCGCGCGGCGAAGTGGGCCGTCTGATGACCCGCGGCCCTTATAC |
| | | CTTCCGCGGCTATTACAACAGCCCGCAACATAACGCTGAAGCCTT |
| | | TGATGCCGATGGATTTTACTGCTCCGGCGATCTGATTTCGATCGAT |
| | | GAAGATGGCTATATCACCGTCCAGGGCCGGGAAAAAGATCAGAT |
| | | CAACCGCGGTGGCGAGAAGATCGCCGCTGAAGAGATAGAAAACC |
| | | TGCTGCTGCGCCATGAGGCGGTGATCCACGCCGCGCTGGTTAGCA |
| | | TTGAAGACAATCTGCTGGGCGAGAAGAGCTGCGCTTATCTGGTGG |
| | | TGACATCGCCGCTGCGCGCCGTCGCGGTGCGCCGCTTCCTGCGCG |
| | | AGCAGGGCGTGGCGGAATTTAAATTGCCGGATCGCGTGGAGTGC |
| | | GTTGCCGCGCTGCCGCTGACGCCGGTGGGCAAAGTCGATAAAAA |
| | | ACAATTACGTCAGTGGCTGGCCGAAGGCAAGCTGGGCTGA |
| Pyoverdine | pvdL | >ADJCKNHF_02470 Enterobactin synthase component B |
| chromophore | | ATGGCAATCCCCAAATTACAGGCTTACGCGCTGCCGGAAGCCAGC |
| precursor | | GATATTCCGGCCAACAAAGTTAACTGGGCCTTTGAGCCGTCGCGC |
| synthetase | | GCCGCGCTGCTGATCCATGATATGCAGGAGTATTTCCTCAACTTC |
| PvdL | | TGGGGCGAAAACAGCGCGATGATGGAAAAAGTGGTGGCCAATAT |
| | | CGCCGCCCTGCGCGACTTCTGCAAACAAAACGGCATTCCGGTGTA |
| | | CTACACCGCCCAGCCGAAAGAGCAGAGCGATGAAGACCGCGCCC |
| | | TGCTGAATGATATGTGGGGGCCGGGGTTGACTCGCTCGCCGGAAC |
| | | AGCAGCAGGTGATTGCCGCGCTGGCGCCGGATGAGCAGGATACG |
| | | GTGCTGGTGAAATGGCGCTACAGCGCGTTTCATCGCTCACCGCTT |
| | | GAAGAGATGCTGAAAGAGACCGGCCGCGACCAGCTGATCATCAC |
| | | CGGCGTTTACGCCCATATCGGCTGTATGACCACCGCCACCGACGC |
| | | TTTTATGCGCGATATCAAACCGTTCTTTGTCGCCGACGCGCTGGC |
| | | AGATTTCAGCCGCGAAGAGCATTTGATGGCGCTGAAATACGTCGC |
| | | CGGCCGTTCTGGACGCGTGGTGATGACCGAAGAATTGTTGCCGCT |
| | | GCCGGCCTCCAAAGCGGCGCTGCGCGCGCTAATTCTGCCGCTGCT |
| | | CGACGAATCCGACGAACCGCTGGATGATGAAAACCTGATCGACT |
| | | ACGGTCTGGATTCGGTGCGTATGATGGCGCTGGCCGCCCGCTGGC |
| | | GCAAAGTACACGGCGATATCGACTTCGTGATGCTGGCGAAAAAC |
| | | CCGACCATCGACGCCTGGTGGGCGCTGCTCTCCCGCGAGGTGAAA |
| | | TAA |
| acetolactate | ilvB, ilvG, | >ADJCKNHF_02673 Acetolactate synthase isozyme 1 large |
| synthase | ilvI | subunit |
| I/II/III large | | ATGGCAAGTTCGGGCACCACATCAAACACAATGCGCTTTACCGGC |
| subunit | | GCGCAGCTGGTTGTTCATTTACTGGAACGCCAGGGTATCACTATG |
| [EC:2.2.1.6] | | GTCAGCGGCATTCCGGGCGGCTCCATCCTGCCTATCTATGATGCC |
| | | TTGAGCCAAAGCACGCAGATCCGCCACATCCTGGCGCGCCACGA |
| | | GCAGGGGGCGGGTTTTATCGCTCAGGGGATGGCGCGTACCGAAG |
| | | GTAAACCCGCAGTCTGCATGGCCTGTAGCGGCCCTGGCGCCACCA |
| | | ACCTGATTACCGCCATCGCCGATGCCCGCCTCGATTCTATTCCGCT |
| | | GGTCTGCATCACCGGGCAGGTTCCGGCCTCAATGATCGGCACCGA |
| | | TGCCTTCCAGGAAGTCGATACCTACGGCATCTCTATCCCCATCAC |
| | | CAAGCATAACTACCTGGTGCGCGACATCGCCGAGCTGCCGCAGGT |
| | | GATGAGCGACGCCTTCCGTATTGCCCAGTCCGGGCGCCCCGGGCC |
| | | GGTGTGGATAGATATTCCTAAGGACGTACAGGCGGCAACCATTG |
| | | AACTGGAAACGCTGCCGGAGCCAGGCGAGCGCGCCCCGGCACCA |
| | | GCATTCGCGCCTGAAAGCGTGCGTGAAGCGGCGGCAATGATCAA |
| | | CGCGGCGAAACGCCCGGTACTGTATCTGGGCGGCGGCGTGATTA |
| | | ACGCGCCTGAGCCGATTCGGGACCTGGCGGAAAAAGCCAACCTG |
| | | CCGACCACCATGACCTTAATGGCGCTGGGTATGTTGCCGAAGGCG |
| | | CATCCTTTGTCGCTGGGCATGCTGGGGATGCACGGCGCGCGCAGC |
| | | ACTAACTTTATTCTGCAAGAAGCTGATTTACTGATTGTTTTAGGCG |
| | | CGCGTTTTGATGACCGGGCGATTGGCAAGACCGAGCAGTTCTGCC |
| | | CGAACGCGAAGATTATTCACGTCGATATCGACCGCTCTGAGCTTG |
| | | GCAAAATTAAGCAACCGCACGTAGCGATTCAGGGCGATGTGGCG |
| | | GAGGTCTTAGCCCAGCTTATTCCGCAGATTGAGGCGCAGCCGCGT |
| | | GATGAGTGGCGCCAGCTGGTGGCTGATTTACAAAGAGAATTCCCC |
| | | TGCGCCATCCCGCAGGAAAGCGATCCGCTCTCCCATTACGGCCTG |
| | | ATTAACGCCGTGGCCGCCTGCGTTGATGATGAGGCGATCATCACC |
| | | ACCGATGTGGGTCAGCATCAGATGTGGACGGCGCAGGCCTATCC |
| | | GCTCAACCGTCCGCGCCAGTGGCTGACTTCCGGCGGTCTCGGCAC |
| | | CATGGGCTTCGGCCTGCCGGCGGCCATCGGCGCGGCGCTGGCCAA |
| | | TCCGCAGCGTAAGGTTATTTGTTTCTCCGGTGACGGCAGCCTGAT |
| | | GATGAATATTCAGGAGATGGCCACCGCTGCCGAAAATCAGCTGG |
| | | ATGTAAAAATTATCCTGATGAACAACGAAGCGCTGGGGCTGGTG |
| | | CATCAGCAGCAGAGCCTGTTCTATCAGCAGGGCGTCTTCGCCGCG |
| | | ACCTATCCGGGGATGATTAACTTTATGCATATAGCTGCCGGTTTT |
| | | GGTCTGCAAACCTGTGATTTAAATAACGAATCCGATCCGCAGGCG |
| | | GCGCTGCAGGCGATTATCAACCGCCCAGGTCCGGCGTTGATCCAC |
| | | GTGCGTATCGACGCGCAGCAAAAAGTGTATCCGATGGTACCGCC |
| | | GGGTGCGGCCAATACTGAGATGGTGGGGGAATAA |
| acetolactate | ilvH, ilvN | >ADJCKNHF 02674 Acetolactate synthase isozyme 1 small |
| synthase I/III | | subunit |
| small subunit | | ATGCAAAAGCAACACGATAACGTCATTCTGGAACTCACCGTCCGC |
| [EC:2.2.1.6] | | AACCACCCAGGTGTGATGACCCACGTCTGCGGGCTATTTGCCCGG |
| | | CGCGCGTTTAACGTTGAAGGCATTCTCTGCTTGCCGATCCAGGGC |
| | | AGCGAGTACAGCCGCATCTGGCTGCTGGTAAATGATGACCAGCG |
| | | GCTGGGGCAGATGATTAGCCAGATTGAAAAGCTGGAAGATGTCA |
| | | CCAATGTGGCGCGTAACCAGTCCGATCCCACCATGTTTAACAAAA |
| | | TTGCGGTGTTCTTCGAATAG |
| ferredoxin- | nasB | >ADJCKNHF_03005 Nitrite reductase [NAD(P)H] |
| nitrate | | ATGAGCAAAGTCAGAATCGCTATTATCGGTAACGGCATGGTCGGC |
| reductase | | CATCGCTTTATCGAAGAGCTTCTTGATAAGGCGCCTGCCGGACAA |
| [EC:1.7.7.2] | | TTCGACATTACCGTGTTCTGTGAAGAGCCGCGTATCGCCTATGAC |
| | | CGTGTCCACCTGTCGTCTTACTTCTCCCATCACACTGCGGAAGAG |
| | | CTGTCGCTGGTACGCGAAGGTTTCTATGAGAAACACGGCGTCAAG |
| | | GTACTGGTCGGCGAACGCGCGATTACCATCAACCGTCAGGAAAA |
| | | GGTGATCCACTCCAGCGCTGGCCGTACCGTTTTCTACGACAAGCT |
| | | GATTATGGCGACTGGCTCATACCCGTGGATCCCGCCCATTAAAGG |
| | | CGCGGAAACCCAGGATTGCTTCGTCTATCGTACTATTGAAGATCT |
| | | TAACGCCATCGAATCCTGCGCCCGTCGCAGCAAACGCGGCGCGGT |
| | | GGTCGGCGGCGGTCTGCTCGGCCTGGAAGCGGCTGGCGCGCTGA |
| | | AAAATCTCGGCGTGGAAACCCACGTGATCGAGTTTGCGCCGATGC |
| | | TGATGGCCGAGCAGCTCGACCAGATGGGCGGCGAGCAGCTGAAG |
| | | CGTAAGATTGAAAGCATGGGCGTGAAGGTTCACACCAGCAAAAA |
| | | TACCAAAGAGATCGTTCAGCAGGGCACCGAGGCACGCAAAACGA |
| | | TGCGCTTCGCCGACGGTAGCGAACTGCAGGTCGATTTCATCGTCT |
| | | TCTCTACCGGTATCCGTCCGCGCGACAAGCTGGCTACCCAGTGCG |
| | | GTCTCGCCGTCGCCCAACGCGGCGGCATCATGGTCAACGATAGCT |
| | | GCCAGACCTCCGATCCGGATATCTACGCTATCGGCGAATGCGCCA |
| | | GCTGGAATAACCGCGTATACGGTCTTGTCGCCCCGGGCTACAAAA |
| | | TGGCGCAGGTTACCGTTGACCATATCCTCGGCAACGACAATCTGT |
| | | TCACCGGCGCCGACCTCAGCGCCAAGCTGAAGCTGCTCGGCGTGG |
| | | ACGTTGGCGGTATCGGCGACGCCCACGGGCGCACGCCGGGCGCG |
| | | CGTAGCTACGTCTATCTCGACGAAAGCAAAGAGGTCTACAAACG |
| | | GCTCATTGTCAGCGAAGATAACAAAACCCTGCTTGGCGCGGTACT |
| | | GGTCGGCGACACCAGCGATTACGGCAACCTGCTGCAGCTGGTGCT |
| | | GAATGCCATCGAGCTGCCGGAAAACCCGGATTCGCTGATCCTCCC |
| | | GGCCCACGCCGGCAGCGGCAAGCCGTCCATCGGCGTGGATAAAC |
| | | TGCCGGACAGCGCGCAGATTTGTTCCTGCTTCGACGTCAGCAAAG |
| | | GCGACCTGATCGCCGCTATCAATAAAGGCTGCCACACCGTGGCGG |
| | | CGCTGAAAGCGGAAACCAAAGCCGGAACCGGCTGCGGCGGCTGT |
| | | ATTCCGCTGGTGACGCAGGTGCTCAACGCCGAGCTGGCGAAACA |
| | | GGGTATCGAAGTGAACAACAATCTGTGTGAGCACTTCGCCTACTC |
| | | TCGCCAGGAGCTGTTCCACCTGATCCGCGTCGAAGGCATCAAAAC |
| | | CTTCGACGAACTGCTGGAAAAACATGGTCAGGGCTACGGCTGTG |
| | | AAGTCTGTAAGCCGACCGTCGGTTCCCTGCTGGCCTCCTGCTGGA |
| | | ATGAGTACATCCTCAAACCACAGCACACGCCGCTGCAGGACACC |
| | | AACGATAACTTCCTCGCCAACATCCAGAAAGATGGCACCTACTCG |
| | | GTTATCCCGCGCTCCGCAGGCGGCGAAATTACGCCAGAAGGCCTG |
| | | GTTGCCGTCGGTCGCATCGCGCGCGAATTTAATCTGTATACCAAA |
| | | ATCACCGGCTCCCAGCGTATCGGCCTGTTCGGCGCGCAAAAAGAC |
| | | GATCTGCCGGAAATCTGGCGTCAACTGATTGAAGCCGGCTTCGAA |
| | | ACCGGCCATGCCTACGCCAAAGCGTTACGTATGGCGAAAACCTGC |
| | | GTCGGCAGTACCTGGTGCCGCTACGGCGTCGGCGATAGCGTCGGC |
| | | TTCGGCGTAGAACTGGAAAACCGCTATAAAGGTATCCGTACCCCG |
| | | CACAAAATGAAGTTCGGCGTCTCCGGTTGTACCCGTGAATGCGCG |
| | | GAAGCCCAGGGTAAAGACGTTGGGATCATCGCCACCGAGAAAGG |
| | | CTGGAACCTGTACGTGTGCGGTAACGGCGGGATGAAACCACGCC |
| | | ACGCCGATCTGCTGGCCGCGGACCTCGATCGCGATACGCTCATCA |
| | | AATATCTCGACCGCTTTATGATGTTCTACATCCGCACCGCCGATA |
| | | AGCTTACCCGTACCGCGCCGTGGCTGGACAACATGGAAGGCGGT |
| | | ATCGACTATCTGCGCAGCGTCATCATCGACGATAAGCTGGGCCTG |
| | | AACGATCACCTTGAAGAAGAGCTGGCTCGCCTGCGCGCCGCTTTT |
| | | GCCTGCGAATGGACCGAGACCGTCAATAACCCGGCGGCGCAGAC |
| | | GCGCTTCAAACACTTTATCAACAGCGATCAGCGCGACCCGAACGT |
| | | TCAGGTGGTGCCGGAACGTGACCAGCATCGCCCGGCCACGCCCTA |
| | | TGAGCGCATCCCGGTCACGCTGGTGGAGGAAAACGTATGA |
| chemotaxis | motA | >ADJCKNHF_03338 Motility protein A |
| protein MotA | | GTGCTAGTGATATTGGGTTATCTCGTGGTCCTGGGAGCGGTATTT |
| chemotaxis | | GGCGGCTATTTACTGGTGGGCGGCCACCTTGGCGCGCTGTACCAG |
| | | CCGGCGGAATTCCTGATTATCGGCGGCGCCGGCATCGGCGCGTTT |
| | | ATCGTCGGCAACAACGGAAAGGCCATCAAATCCACGCTGCGGGC |
| | | CTTGCCCAAAATGGTACGTCGCTCCAAATACAGCAAGGCGCTGTA |
| | | TATGGATCTGATGGCGTTGCTGTTCCTGCTGCTCGCAAAATCCCGT |
| | | CAACAGGGAATGCTGTCGCTTGAGTTCGATATCGACAATCCTCAG |
| | | GAAAGTGAAATTTTCGCTAATTATCCGCGCATCCTTGCCGATAAC |
| | | CATCTGGTGGAGTTCATAACCGATTATTTACGCCTGATGGTGAGC |
| | | GGCAACATGAATGCTTTTGAAATTGAAGCGCTGATGGACGAAGA |
| | | GATCGAAACCTTCGAACAGGAGAGCGAAGTGCCCGCCGGCAGCC |
| | | TGGCGATGGTCGGCGACTCTCTGCCGGCATTTGGTATTGTCGCGG |
| | | CGGTAATGGGGGTGGTGCACGCGCTGGCCTCGGCGGACCGTCCG |
| | | GCGGCAGAGCTCGGGGCGCTTATCGCCAACGCGATGGTGGGGAC |
| | | TTTCCTCGGCATTCTGCTGGCCTATGGATTTATCTCGCCGCTGTCG |
| | | ACGCTGCTGCGGCAAAAAAGCGCCGAGACGGTCAAGATGATGCA |
| | | GTGCATCAAGGTGACATTGCTCTCCAGCCTCAACGGCTACGCGCC |
| | | GCAAATCGCCGTCGAATTTGGCCGCAAAACGTTATACACCACCGA |
| | | GCGCCCGTCGTTTGTCGAGCTTGAAGAACACGTACGCCAGGTGAA |
| | | AGCGCCTGCCGCGCAGGCGACGGAAAGCGAAGAAGCATGA |
| protein MotB | motB | >ADJCKNHF_03339 Motility protein B |
| | | ATGAAGCATAATCACCCGGTGGTTCTGGTCAGAAAGCGCAAATC |
| | | ACACCAGCCAGCCCATCACGGCGGTTCGTGGAAGATTGCCTATGC |
| | | CGATTTTATGACGGCGATGATGGCCTTCTTCCTTGTGATGTGGCTG |
| | | CTGGCGATCGCCAGCCCGCAGGAGCTGACGCAAATAGCCGAATA |
| | | TTTCCGTACGCCGCTGAAAGTCGCCCTCACCAGCGGCGATAAAAG |
| | | CAGCTCGGAAAGCAGCCCGATCCCCGGCGGCGGCGAAGACCCGA |
| | | CCCGTGAAGTCGGGTTAGTCAGCAAGCAGATCAATACCGCGGAT |
| | | AAACGTGCCGAAGAGCTACGGCTAAACAAACTGCGTGACAAGCT |
| | | CGATCAGTTGATTGAGTCAGACCCGCGCCTGAAGGCGCTACGTCC |
| | | GCATCTGCTGATCAACATGATGGACGAAGGACTGCGCATTCAGAT |
| | | TATCGATAGCCAGAATCGGCCGATGTTTAAAACCGGTAGCGCTCA |
| | | GGTGGAAAGCTATATGCGCGATATCCTGCGAGCGATTGCGCCAAT |
| | | CCTCAATGATTTGCCGAACAAGATAAGCCTCGCCGGGCATACCGA |
| | | TGACATCCCCTACGCCAGCGGCGAACGTGGTTACAGCAATTGGGA |
| | | ACTGTCGGCGGATCGCGCCAACGCCTCGCGCCGCGAGCTGATCGC |
| | | CGGCGGGCTGGCGGAAGGGAAGGTGCTGCGGGTGGTTGGTATGG |
| | | CGGCGACCATGAGCCTGAAACAGCACGGCGCCGATGATGCCATC |
| | | AACCGCCGCATTACCGTACTGGTGCTTAACAAAGAGACCCAGCA |
| | | GAGCATTGAGCATGAAAATGGCGAAAGCAATGCGCTGGAGATTA |
| | | GCCAGCCGGATACGCTGCCAAAGCTGGTCGCGCCCGCGCCGGTTA |
| | | ACCGCGATTCACACCCTGAGGTGACCCCATGA |
| two- | cheA | >ADJCKNHF_03340 Chemotaxis protein CheA |
| component | | ATGAGCATGGATATTAGCGCTTTTTATCAGACTTTTTTTGATGAAG |
| system, | | CAGATGAGCTGCTGGCAGACATGGAGCAACACCTGCTGGAGCTG |
| chemotaxis | | GATCCGCAGGCCCCCGATATCGAACCGCTAAACGCTATTTTTCGC |
| family, sensor | | GCGGCGCACTCGATCAAAGGCGGCGCGGCGACGTTTGGCTTTTCC |
| kinase CheA | | GTATTGCAGGAAACCACCCATCTGCTGGAGAACCTGCTCGACGGC |
| [EC:2.7.13.3] | | GCCCGGCGCGAGGAGATGCGCTTGAGCACCGAAATTATCAATCT |
| | | GTTTCTGGAAACCAAAGATATTATGCAGGAGCAACTGGACGCCTA |
| | | TAAAACCTCGCAGCAGCCTGACGCTGAGAGCTTTGACTATATCTG |
| | | CCAGGCGCTGCGGCAGCTGGCGCTGGAAGCGCAGCAGCAGGACG |
| | | CGCCAGCCGCGCCGCCCGTCGTGGCCCAGCCTGCGCCGACGGCCG |
| | | TCGCCGGCGGTATGCGCGTGAGTTTAACCGGGCTTAAAGCCAATG |
| | | AAATTCCGCTGATGCTGGAAGAGCTTGGCAATCTCGGCGAGGTAC |
| | | ATGATCCGCAGCAAACTGACAATAGCCTTGAGGTGACCCTGCTGA |
| | | CCACCGCCAGCGAAGAAGATATTTGCGCGGTGCTCTGTTTCGTTC |
| | | TTGAGCCGGAACAGATCAGCTTTACCACGCCGCCAACCACTGCCG |
| | | CTAAACCATTGCCATCGGCCGAAGTTGTGCCGCCGCCGGTCGCCC |
| | | AACCGCAGCCTGCTGTCGTCGAGCCGCCGAAAGCGCCGAGAGCG |
| | | AAGGCCAGCGAATCGACCAGTATTCGCGTGGCGGTAGAGAAAGT |
| | | TGACCAGTTGATTAACCTCGTCGGCGAACTGGTGATCACCCAGTC |
| | | CATGCTGGCGCAGCGCTCAGGTAACCTGGATCCGGTGACCCATGG |
| | | CGATCTGCTCAACAGCATGAGCCAGCTGGAACGTAACGCTCGCG |
| | | ACCTGCAGGAATCGGTGATGTCGATTCGCATGATGCCGATGGAAT |
| | | ATGTATTCAGCCGCTACCCGCGGCTCGTGCGCGACCTGGCCGGCA |
| | | AGCTCAATAAGCAGGTGGAACTGACGCTGCAGGGCAGCTCCACC |
| | | GAACTTGATAAGAGCCTGATCGAGCGCATTATCGACCCGTTAACC |
| | | CACCTGGTGCGCAATAGCCTCGACCACGGTATTGAAGATCCGCAA |
| | | ACCCGTCTGGCGGCAGGTAAGTCTGAGGTGGGCAATCTGATTCTT |
| | | TCCGCTGAACATCAGGGCGGCAATATCTGCATCGAAGTGATCGAT |
| | | GACGGCGCCGGGCTCAATCGCGAAAAAATTCTCGCCAAAGCGGC |
| | | GGCGCAGGGGCTGGCGGTCAGCGACAGCATGAGTGATGAAGAGG |
| | | TCGGAATGCTTATTTTTGCGCCGGGCTTTTCGACCGCGGAACAGG |
| | | TGACCGACGTCTCTGGCCGCGGCGTCGGCATGGACGTCGTGAAAC |
| | | GGAACATCCAGGAGATGGGCGGGCACGTTGAAATCCATTCCCGC |
| | | GCGGGCAAAGGGACCTCGATTCGTATTTTGCTGCCGCTGACGCTC |
| | | GCTATCCTCGACGGCATGTCGGTCAAGGTCAATGAAGAGGTCTTT |
| | | ATTCTGCCGCTCAACGCGGTCATGGAATCGCTGCAACCGCAGGCC |
| | | GAAGACCTGCATCCGATGGCCGGCGGCGAGCGGATGCTGCAGGT |
| | | TCGCGGCGAGTATCTACCGCTGGTGGAGCTCTACCGGGTGTTTGA |
| | | TGTCGCCGGGGCGAAAACCGAGGCCACTCAGGGCATCGTGGTGA |
| | | TTCTGCAAAGCGCCGGCCGCCGCTATGCGCTGCTGGTGGATCAGC |
| | | TGATCGGCCAGCACCAGGTGGTGGTGAAAAACCTGGAAAGCAAT |
| | | TACCGCAAAGTGCCGGGAATTTCCGCGGCGACGATCCTCGGTGAC |
| | | GGCAGCGTGGCGCTGATCGTCGACGTGTCGGCGCTGCAAATGCTC |
| | | AATCGGGAAAAGCTGCTGAGCGCAGCGGCCGCATAA |
| purine- | cheW | >ADJCKNHF_03341 Chemotaxis protein CheW |
| binding | | ATGGCAGGATTAGCAACCGTCAGCAAATTGGCTGGCGAAACGGT |
| chemotaxis | | AGGTCAGGAGTTTTTAATCTTTACCCTCGGCAATGAAGAATACGG |
| protein | | CATCGATATTCTGAAAGTGCAGGAGATCCGCGGCTATGACCAGGT |
| CheW | | GACGCGTATCGCCAACACCCCGGATTTCATCAAAGGCGTCACCAA |
| | | TCTGCGCGGGGTGATCGTGCCGATTATCGACCTGCGGGTAAAATT |
| | | CGCCCAGCAGGGCGTCTCTTATGATGAAAACACGGTGGTTATCGT |
| | | GCTTAACTTCGGCCAGCGGGTGGTGGGGATTGTGGTCGACGGCGT |
| | | CTCTGACGTGTTGTCTCTCACCGCCGAACAGATCCGCCCGGCGCC |
| | | GGAATTCGCGGTAACGCTGGCGACCGAGTATCTCACCGGTCTTGG |
| | | CGCGCTCGGCGAGCGTATGTTGATTCTCGTCGATATCGAAAAGCT |
| | | GCTCAGCAGCGAAGAGATGGCGCTGGTCGATAACGTCGCCAAAA |
| | | GCCACTAA |
| chemotaxis | cheR | >ADJCKNHF_03344 Chemotaxis protein methyltransferase |
| protein | | ATGAAGCAGACGACATCAACCGCGGCGCGTGAAAGCGGATCGGC |
| methyltransfe | | GCTGGCGCAGATGGTTCAGCGTCTGCCGCTCTCCGACGCGCATTT |
| rase CheR | | TCGCCGCATCAGCCAGCTTATCTATCAGCGTGCCGGGATCGTGCT |
| [EC:2.1.1.80] | | GGCGGCGCATAAGCGCGAGATGGTGTACAACCGGCTGGTGCGCC |
| | | GTTTGCGTCTGCTGGGCATTCATGATTTCGGCGACTACCTGGCGCT |
| | | GCTGGAAAGCGACCCGCACAGCGCCGAGTGGCAGGCGTTTATCA |
| | | ATGCGCTGACCACCAACCTGACCGCCTTTTTTCGCGAGGCGCACC |
| | | ACTTTCCGATTCTGGCGGAGCATGCGCGCTCGCGTCCCGGGAACT |
| | | ATAGCGTGTGGAGCACCGCCGCCTCGACCGGCGAAGAACCTTATT |
| | | CCATCGCCATTACGCTCGGTGATGCGCTCGGGGAACGTGCGGGCA |
| | | GTTGCCAGGTCTGGGCCAGCGATATTGACACCCAGGTGCTGGAGA |
| | | AAGCGGAAGCAGGGATTTATCGCCATGAAGATCTGCGCACTCTG |
| | | ACGCCAATCCAGATGCAGCGTTATTTTCTGCGCGGCACCGGGCCG |
| | | CATCAGGGGCTGGTGCGCGTGCGTCAGGAGCTGGCGGCGCGGGT |
| | | CAACTTCCAGCCGCTGAATCTGCTGGCGGCGGAGTGGGCGCTGCC |
| | | GGGGCCGTTCGACGCGATTTTTTGCCGCAACGTGATGATCTATTT |
| | | CGATAAACCGACGCAGGAACGCATCCTGCGCCGCTTTGTCCCCTT |
| | | GCTTAAACCGGGGGGGCTGTTGTTTGCCGGTCACTCCGAGAATTT |
| | | CAGCCAGATCAGCCGGGATTTCTACCTGCGTGGACAGACCGTGTA |
| | | TGGGCTGGCCAAGGAGAAGTAA |
| two- | cheB | >ADJCKNHF_03345 Protein-glutamate methylesterase/protein- |
| component | | glutamine glutaminase |
| system, | | ATGAGTAAAATCAGAGTGTTATGTGTTGATGATTCGGCCCTGATG |
| chemotaxis | | CGCCAGTTGATGACCGAGATCGTCAATGGCCACGCCGATATGGA |
| family, | | GATGGTGGCGGTGGCCCCGGACCCGCTGGTCGCACGGGATCTGAT |
| protein- | | CAAAAAATTTAACCCACAGGTGCTGACGCTGGACGTCGAAATGC |
| glutamate | | CGCGCATGGACGGCCTCGATTTCCTCGAAAAGCTGATGCGCCTGC |
| methylesterase/ | | GGCCGATGCCGGTGGTGATGGTTTCATCGCTGACCGGTAAAGGTT |
| glutaminase | | CGGAAATTACCCTGCGCGCGCTGGAGCTGGGGGCGGTGGATTTCG |
| | | TCACCAAACCGCAGCTCGGGATCCGCGAAGGCATGCTGGCTTACA |
| | | GCGAACTGATAGGCGAAAAGATCCGTACCGCCGCACGGGCGCGG |
| | | CTACCGCAGCGCGCCAATGACCAGCCGCCGGCCATTTTAAGCCAC |
| | | GGACCGCTGCTGAGCAGCGAGAAGCTGATCGCCATTGGCGCTTCC |
| | | ACCGGCGGCACCGAAGCGATCCGTCAGGTGCTACAACCGTTGCC |
| | | GGCCACCAGTCCGGCGCTGCTGATCACCCAGCATATGCCGCCGGG |
| | | ATTTACCCGCTCGTTTGCCGAACGGTTGAATAAGCTGTGCCAGAT |
| | | CACGGTGAAAGAGGCGGAAGAGGGCGAACGCGTGCTCCCGGGAC |
| | | ACGCCTATATTGCGCCTGGGGATCGCCATCTGGAGCTGGCGCGTA |
| | | GCGGCGCTAACTATCAGGTCAAGCTGCACGACGGCCCGGCGGTA |
| | | AATCGCCATCGTCCCTCGGTGGATGTGCTGTTTCGTTCGGTGGCCC |
| | | GCCACGCCGGGCGCAATGCGGTGGGGGTGATCCTCACCGGCATG |
| | | GGCAACGACGGCGCGCAGGGGATGCTGGAGATGCATCGCGCCGG |
| | | GGCCTATACCCTGGCGCAGAGTGAGGCGAGCTGCGTGGTGTTCGG |
| | | CATGCCGCGCGAGGCCATCGCCAGCGGCGGGGTCAACGAAGTGG |
| | | TTGAACTGGAGCGCATGAGCCAACGCATGCTGGCGCAGATAGCC |
| | | GGCGGCCAGGCGCTGCGAATTTAA |
| two- | cheY | >ADJCKNHF_03346 Chemotaxis protein CheY |
| component | | ATGGCAGATAAGAATCTCCGTTTTCTGGTCGTCGACGACTTTTCC |
| system, | | ACCATGCGTCGTATCATCAGAAATTTGCTTAAAGAGCTCGGCTTC |
| chemotaxis | | AACAACGTCGAAGAGGCGGAAGATGGCGCGGACGCGCTAAATAA |
| family, | | GCTGCGAGCCAGCAGCTTTGATTTCGTGGTCTCCGACTGGAACAT |
| chemotaxis | | GCCTAACGTTGACGGCCTGGAGCTGCTGCAGACCATTCGTGCCGA |
| protein CheY | | TGCCGCGCTGGCGGCGATGCCGGTATTGATGGTGACGGCGGAAG |
| | | CGAAAAAAGAAAATATTATTGCCGCCGCGCAGGCAGGCGCCAGC |
| | | GGTTATGTGGTGAAACCTTTTACGGCGGCGACGCTGGAAGAAAA |
| | | GCTCAACAAGATTTTTGAAAAATTGGGCATGTAA |
| flagellar | flhB | >ADJCKNHF_03348 Flagellar biosynthetic protein FlhB |
| biosynthetic | | TTGGCGGAAGACAGCGATCTGGAAAAAAGCGAGGCCCCCACCGC |
| protein FlhB | | CCACCGGTTGGAGAAGGCGCGTGAAGAGGGCCAGATCCCGCGCT |
| | | CGCGCGAGCTGACCTCGGTACTCATGCTGGTGGCCGGGCTCGCCA |
| | | TTATCCTGATGTCCGGCAGCAATATCACTCGCCAACTGGCGGAAA |
| | | TGCTGACGCAGGGCCTGCACTTTGACCACGGGATGGTCAGCAATG |
| | | ACAAACAAATGCTGCGCCAGCTGGGAATGCTGCTGCGTCAGGCG |
| | | GTGCTGGCATTGCTGCCGGTAATGGCCGGGCTGGTGCTGGTGGCG |
| | | CTGGCCGCGCCGATGCTGCTTGGCGGCATTTTATTCAGCACCAAG |
| | | TCGCTCAAATTCGATCTTAAACGGCTGAATCCGCTTTCCGGTCTG |
| | | AAACGCATGTTCTCCACCCAGGTGCTGGCGGAGCTGTTAAAAGGG |
| | | ATCCTCAAAGCCACGCTGGTCGGTTGGGTAACCGGGCTGTACCTA |
| | | TGGCACAACTGGGCGGCGATGCTGCATCTGATGACCCAACAGCC |
| | | GCTCGACGCGTTGGCCAATGCGCTGCAGATGATCCTCTATTGCGG |
| | | ATTTCTGGTGGTGCTCGGATTGACGCCGATGGTGGCGTTTGACGT |
| | | TTTTTATCAGCTGTGGAGCCACTTCAAAAAGCTGAAGATGACCAA |
| | | ACAGGATATCCGCGACGAATTCAAAGATCAGGAAGGCGACCCGC |
| | | ATGTTAAAGGGCGGATCCGTCAACAGCAGCGGGCGATTGCCCAG |
| | | CGGCGGATGATGGCCGATGTGCCGAAAGCGGACGTGATAGTCAC |
| | | TAACCCGACCCACTATGCCGTGGCGTTGCAGTACAACGATAAAAA |
| | | AATGAGCGCGCCGAAGGTGTTGGCTAAAGGGGCGGGGGAGATTG |
| | | CGCTGCGCATTCGCGAACTGGGAGCGCAACACCGTATTCCGATGC |
| | | TTGAAGCGCCGCCGCTGGCCCGCGCGCTCTATCGCCATAGCGAGA |
| | | TTGGCCAGCATATTCCCGCCACCCTCTATGCCGCGGTCGCCGAAG |
| | | TGCTGGCCTGGGTTTATCAGCTGCGCCGCTGGCGGCGCGAGGGCG |
| | | GCCTGATCCCGAAAAAACCTCAACGTTTACCGGTGCCGGAAGCAC |
| | | TGGATTTTGCAAAAGAGAGTGACTCTGATGGCTAA |
| flagellar | flhA | >ADJCKNHF_03349 Flagellar biosynthesis protein FlhA |
| biosynthesis | | ATGGCTAATTTGGCCTCCCTGCTGCGTTTGCCGGGGAATGTTAAA |
| protein FlhA | | GATACGCAATGGCAGGTTCTGGCCGGCCCGATCCTGATCCTGCTG |
| | | ATTTTGTCGATGATGGTGCTGCCGCTGCCGGCGTTTATCCTCGACC |
| | | TGCTTTTTACTTTCAATATCGCGTTGTCGATCATGGTGCTGCTGGT |
| | | GGCGATGTTTACCCAGCGCACGCTGGACTTCGCCGCGTTTCCGAC |
| | | CATTCTGCTGTTCTCGACGCTGCTGCGCCTGTCGCTCAACGTCGCC |
| | | TCGACGCGCATCATTTTGATGGAAGGGCACACCGGGGCCGCCGCC |
| | | GCGGGGCGGGTCGTGGAGGCCTTCGGTCACTTCCTGGTCGGCGGT |
| | | AATTTCGCCATCGGTATCGTGGTGTTTATCATCCTCGTGCTGATCA |
| | | ACTTCATGGTTATCACCAAAGGCGCCGGGCGTATCGCCGAAGTGG |
| | | GGGCGCGTTTCGTACTCGACGGCATGCCGGGTAAGCAAATGGCG |
| | | ATCGATGCCGACATGAACGCCGGGCTTATCGGTGAAGAAGAGGC |
| | | GAAAAAACGCCGCGCGGAAGTCACGCAGGAAGCCGATTTCTACG |
| | | GCTCGATGGACGGCGCCAGCAAGTTTGTTCGCGGCGATGCCATCG |
| | | CCGGGCTAATGATTATGGTGATTAACGTGGTCGGCGGACTGCTGG |
| | | TCGGCGTGGTGCAGCACGGTATGGAGCTGGGGGCGGCGGCGGAA |
| | | AGCTACACCTTGTTGACCATCGGCGACGGGCTGGTGGCGCAAATC |
| | | CCGGCGCTGGTGATCTCTACCGCCGCCGGGGTCATCGTGACCCGG |
| | | GTGGCGACCGATCAGGATGTCGGCGAACAGATGGTCGGCCAGCT |
| | | ATTCAATAACCCAAAGGTCATGCTGCTGTGTGCCGCGGTACTTGG |
| | | GCTGCTGGGGCTGGTGCCGGGGATGCCGAACCTGGTCTTCCTGCT |
| | | GTTTACCGCCGCGCTGCTCGGGCTGGCCTGGTGGCTGCGCGGGCG |
| | | CGAAAAACAGGCGCCGAAAGCCGCCGACGAACCCATCGTGCAGG |
| | | AGAATACCCAGGCCGCGGAAGCCAGTTGGGCCGATGTCCAGTTG |
| | | GAAGATCCGCTGGGCATGGAAGTGGGTTACCGGCTGATTCCGATG |
| | | GTCGATTTTCAGCAAAATGGCGAGCTGTTGGGGCGTATCCGCGGT |
| | | ATCCGCAAGAAATTCGCCCAGGAGATGGGCTATCTGCCGCCGGTG |
| | | GTGCATATCCGCGATAACCTCGAACTGGCGCCCGCCCGCTACCGC |
| | | ATTCTGATGAAAGGCGTGGAAATCGGCAGCGGCGAAGCGCAGCC |
| | | TGGGCGCTGGCTGGCGATCAACCCCGGCAACGCTATCGGCGAGCT |
| | | GGCGGGGGATAAAACTATCGATCCGGCCTTCGGTCTGGAGGCGG |
| | | TGTGGATTGATAGCGCGTTGCGCGAGCAGGCGCAAATTCAGGGCT |
| | | ATACGGTGGTGGAGGCCAGTACGGTGGTGGCGACCCATCTCAAC |
| | | CACCTCATCGGCCAGTTCGCCAGCGAACTGTTTGGCCGCCAGGAG |
| | | ACGCAGCAGCTACTCGACCGTGTGTCGCAGGAGATGCCGAAGCT |
| | | GACCGAAGATTTCGTACCGGGCGTGGTATCGCTGACCACGCTGCA |
| | | TAAGGTGTTGCAGAATCTGCTGGCCGAGCGGGTATCGATCCGCGA |
| | | TATGCGTACCATTATCGAGACGCTGGCGGAACATGCGCCGACGCA |
| | | AAGCGATCCTTACGAGCTGACCACCGTCGTGCGCGTGGCGCTGGG |
| | | GCGGGCGATTGCCCAGCAGTGGTTCCCTGGCAATGGTGAAATTCA |
| | | GGTTATCGGCCTGGATACCCAACTGGAACGACTGCTGCTGCAGGC |
| | | GCTGCAGGGCGGCGGCGGTCTCGAACCGGGGCTTGCCGACCGCC |
| | | TGCTGGAGCAGGCGCGGCAGGCGCTGCAGCGCCAGGAGATGCTC |
| | | AGCGCGCCGCCGGTGCTGCTGGTTAACCACGCGCTGCGCCCGCTA |
| | | CTGGCGCGCTTCCTGCGCCGTAGCCTGCCGCAGATGGCGGTGCTT |
| | | TCCAACCTGGAGATCAACGACGATCGCCAGATTCGCATGACCTCA |
| | | ACCATTGGAGCGGCCTGA |
| flagella | flgN | >ADJCKNHF_03351 Flagella synthesis protein FlgN |
| synthesis | | ATGGACAGATTACGCACTCTGCTGGATAAACTGGCGGAAACCCTG |
| protein FlgN | | CGGGCGCTGGATGACGTGCTGGCGCAAGAGCAACAATTGCTTTGT |
| | | GCGGATGAACTGCCCGGCGTGGCGCTGCAGCGCGTCACCGATAG |
| | | CAAGAGTCAACTGCTGGCGACGGTGGCCTGGCTTGAACAACAGC |
| | | GCCCGGCGCTGGAAGCCAACCTTGCGCTACGCGCGCCTTACCCCG |
| | | GCCATGAAAGCTTCAGCGAACGCTGGCGGCAGATCCAGCAGTTA |
| | | AGCCAGAGCCTGCGTGAAAAGAATCAGCATAACGGCCTGTTGCT |
| | | CAATCAACAGATCGCCCATAACCAGCAGGCGCTGGCAATTCTTAG |
| | | CAAGAATAATAAATCGCTGTACGGGCCGGACGGTCAGTCGCGCG |
| | | CCGCCAGTCTGCTGGGCCGTAAAATCGGCGTCTGA |
| negative | flgM | >ADJCKNHF_03352 Negative regulator of flagellin synthesis |
| regulator of | | ATGAGTATCGATCGCACCCAGCGGTTACAGCCGGTTTCCACGGTG |
| flagellin | | CAGCCGCGTGAAACCCCGGCCGACAACCCGCTTCAGCCGCGCAA |
| synthesis | | GGCCACCGCGGCGGAAACCGCCGTTAGCGCCACCAAAGTGAAAC |
| FlgM | | TGAGCGATGCGCAGGCGCGGCTGATGCAGCCCGGCACCCAGGAT |
| | | ATTGATATGAACCGGGTCGAGGCTATCAAACAGGCGATTCGTAGC |
| | | GGCGAACTGAAAATGGACGCGGGCAAAATCGCCGACGCCCTGCT |
| | | GCAGGATGCGCACAACGATATCCAGCGCCTTGCCGGTAGCAAAA |
| | | CAGATAAGGCCTGA |
| flagellar | flgB | >ADJCKNHF_03354 Flagellar basal body rod protein FlgB |
| basal-body | | ATGCTCGACAAACTGGACGCTGCTCTGCGTTTTGGCCAGGAGGCG |
| rod protein | | CTGAACCTGCGCGCCCAGCGCCAGGAAATACTGGCCGCCAATATC |
| FlgB | | GCCAACGCAGACACGCCGGGCTATCAGGCGCGGGATATCGATTT |
| | | CGCCAGCCAACTGAATAAAGTCCTGCAACAGGGGGGGGTAAACG |
| | | GCAACGGCATGGCGCTGAACCTGACGGCGGCGCGTCATATTCCG |
| | | GCGCAGACCATGCAGCCGCCGGAGCTGGATCTGCTGTTCCGGGTG |
| | | CCGGACCAGCCGTCGATGGACGGCAACACGGTGGACATGGATCG |
| | | TGAACGCACCAACTTCGCTGACAACAGCCTGAAATATCAGACCG |
| | | ACCTGACGCTGCTCAACGGTCAGATCAAAGGGATGATGTCGGTGC |
| | | TGCAACAAGGATAA |
| flagellar | flgC | >ADJCKNHF_03355 Flagellar basal-body rod protein FlgC |
| basal-body | | ATGTCTTTACTCAATATTTTTGATATCTCCGGCTCGGCGCTCTCGG |
| rod protein | | CCCAGTCACAGCGGATGAATGTCAGCGCCAGCAATATGGCCAAC |
| FlgC | | GCCGATAGCGTCACCGGCCCGGATGGCGAACCCTATCGCGCGAA |
| | | GCAGGTGGTGTTCGAAGTGGCCGCGGCACCAGGGCAGCAGACAG |
| | | GCGGCGTGCGCGTCTCGCAGGTGGTCGATGACCCGGCGCCCGCGC |
| | | GGATGGTTTATCAACCGGGTAATCCGCTGGCCGATGCTAAGGGTT |
| | | ATGTACGGATGCCGAACGTCGATGTGGTAAGCGAAATGGTTAAC |
| | | ACCATCTCCGCCTCCCGCAGCTATCAGGCCAACGTCGAAGTGCTC |
| | | AACACCACTAAGTCGATGATGATGAAAACCCTGACGTTGGGTCA |
| | | GTAA |
| flagellar | flgD | >ADJCKNHF_03356 Basal-body rod modification protein FlgD |
| basal-body | | ATGGCTATTGCCGCAACCACCAATGAATCGACCAACAACGCGGTT |
| rod | | CTTGATTCCGCCAGCAAAAGCAGCACCAGCCAGGATCTGCATAAC |
| modification | | AGCTTCCTGACGTTGCTGGTGGCGCAGTTGAAAAATCAGGACCCG |
| protein FlgD | | ACCAACCCGATGCAGAACAACGAGCTGACCTCGCAGCTGGCGCA |
| | | GATTAATACCGTACAGGGCATCGAAAAACTCAACACCACCCTTGG |
| | | CGCGATCTCCGGGCAGATCAACAGCAACCAGTCGCTGCAGGCCA |
| | | GCGCGCTGATCGGCCACGGCGTGATGGTACCGGGTAATAACATTC |
| | | TGGTCGGCAGCAAAGAAGGCCAGGTCAGCACCACGCCGTTTGGC |
| | | GTCGAACTGGAGCGCGCCGCCGATCAGGTCACGGCAACCATCAC |
| | | CAACGCCAACGGCCAGGTGGTGCGGACCATTGAGATTGGCGGCC |
| | | TCACCGCCGGGGTGCACGCCTTCACCTGGGACGGCTCGCTGGATG |
| | | ATGGTACCGCCGCGCCGGACGGCGCCTATAAAGTGGCGATTAAC |
| | | GCCAAGGGCAACGGCGAGCAGCTGGTGGCGCGTAGCCTGCATTT |
| | | CGGCATGGTTAACGGTGTGATTAACGACAGCAACGGCGCGAAGC |
| | | TGGATCTCGGCCTGGCCGGTAACGCCAGCCTCGAAGAAGTGCGG |
| | | CAGATCCTGTAA |
| flagellar hook | flgE | >ADJCKNHF_03357 Flagellar hook protein FlgE |
| protein FlgE | | ATGGCCTTTTCTCAGGCAGTCAGCGGCTTAAATGCGGCGGCCACC |
| | | AATCTCGACGTGATTGGCAACAATATCGCTAACTCCGCGACCGCG |
| | | GGTTTTAAATCCGGCAGCGTGTCGTTCGCCGATATGTTCGCCGGT |
| | | TCGCAGGTCGGGCTGGGGGTTAAAGTTTCCGGGATCACCCAGAAC |
| | | TTTAAAGGCGGCACCACTACCGGCACCAGCCGCGCGCTGGATGTG |
| | | GCGATTAACGGCAACGGCTTCTTCCGCATGCAGGATAAAGACGG |
| | | CGGTATTTTCTATACCCGCAACGGCCAGTTTAAGCTGGACGAAAA |
| | | CCGCAATCTGACCAATATGCAGGGGCTGCAGCTGACCGGCTATCC |
| | | GGCCGCCGGCACGCCGCCGACCATTCAACAGGGCGCCAACCCGG |
| | | TACCGCTGAGCATTCCGGAAGGAATGATGAACGCCAAAGCGTCG |
| | | ACTTCCGGCGAAATGGTCGCCAATCTGAAGTCGACGCATAAAGTG |
| | | CCGGAGAACAAGACCTTCGACCCGGCGAAGCAGGACAGCTATAA |
| | | CTACGTCAACACCATCACCGCCTACGATTCGCTGGGTAACGCCCA |
| | | CAACATCAACGCCTATTTCGTGAAAACCGACGATAACAAATGGC |
| | | AGGTCTATACCCAGGACGGCAGCGCGGCTGCGGTCAGCGCGGGC |
| | | ACCATGGAGTTCAATACCAGCGGCAACCTGGTCAGCGTTAACGGC |
| | | CAGGCGGGCGAGTTCAGCATGACCATTCCGATGAGCGCGAAAGA |
| | | CGGCGCCCCGGCGCAGAACTTCACGCTCAGCTTCGCCGGTAGTAT |
| | | GCAGCAGAACGTCGGTAGCGATTCGGTGAGTAAAGTGGCGCAGG |
| | | ATGGTTATGCCGCCGGTGAATACACCAACTTCCAGATTAACGATG |
| | | ACGGTACCGTCGTCGGTATCTATTCCAACCAACAGACCCAGGTCC |
| | | TGGGGCAGATTGTGATGGCCAACTTCTCCAACCCGGAAGGGCTGG |
| | | CGTCGCAGGGCGATAACGTCTGGCAGGAGACCGGCGCCTCCGGC |
| | | CAGCCACGGGTGGGACTCTCCGGCGGCGGCGGTTTTGGCAAGCTG |
| | | ACCAGCGGCGCGCTGGAGTCTTCCAACGTCGACCTGAGCCAGGA |
| | | GCTGGTGAACATGATTGTCGCCCAGCGTAACTATCAGTCCAACGC |
| | | CCAGACCATCAAGACGCAGGATTCGATCCTGCAGACGCTGGTCA |
| | | GCCTGCGCTAA |
| flagellar hook | flgE | >ADJCKNHF_03358 Flagellar basal-body rod protein FlgF |
| protein FlgE | | ATGGATCACGCGATTTATACCGCGATGGGCGCCGCGCGCCAGAC |
| | | GCTGGAGCGACAGTCGATTACCGCCAACAATCTCGCCAACGCCTC |
| | | GACGCCGGGCTTTCGCGCCCAGCTCGCCGCGCTGCGCGCGGTGCC |
| | | GGTTGACGGGCCGAGCCTCGCCACCCGCACGCTGGTGGCGGCCTC |
| | | GACGCCGGGCGCCGATATGAGCCAGGGGGCGCTGAACTACACCG |
| | | GGCGCCCGCTGGACGTGGCGCTGCAGCAGGACGGTTTTCTGGCGC |
| | | TCAGCCTGCCGGGCGGCGGCGAAGCCTATACCCGCAACGGCAGC |
| | | ATTCAGGTTTCGGCTAACGGTCAGTTGACGGTGCAGGGAATGCCG |
| | | CTGCAGGGCGACGGCGGGCCGATTGAGGTACCGCCATCGGCGGA |
| | | AATCACTATCGCGGCCGACGGCACCATTTCGGCGCTGAATCCCGG |
| | | CGACCCGCCGAACACCATCGCGCAGATTGGCCGCCTGAAGCTGGT |
| | | GAAAGCTGATGCGCGCGAAGTGATGCGCGGCGATGACGGCCTGT |
| | | TCCGCCTGACCCCGGAAACCCAGCAGCGCCGCGGCAATCTGTTGG |
| | | CCAACGACCCGCAGGTGCGGGTCATGCCGGGCGTGCTGGAGGGC |
| | | AGTAACGTCAAGCCGATGGAGACCATGGTCGATATGATCGCCAA |
| | | CGCCCGTCGCTTTGAAATGCAAATGAAGGTCATCCACAGCGTGGA |
| | | TGAGAACGAACAGCGGGCGAACTCTCTGCTGTCAGTAAGCTGA |
| flagellar | flgK | >ADJCKNHF_03359 Flagellar basal-body rod protein FlgG |
| hook- | | ATGATTCCCTCTTTATGGATTGCGAAAACCGGTCTTGATGCGCAG |
| associated | | CAGACCAATATGGACGTGATCGCCAACAACCTGGCGAACGTCAG |
| protein 1 | | CACCAACGGTTTTAAACGCCAGCGCGCGGTGTTTGAAGATCTGCT |
| FlgK | | GTATCAGACCATGCGCCAGCCGGGGGCGCAGTCCTCCGAGCAGA |
| | | CCACGCTGCCTTCCGGCCTGCAGATCGGTACCGGCGTGCGCCCGG |
| | | TCGCCACCGAGCGTCTGCATAGCCAGGGCAACCTGTCGCAGACCA |
| | | ACAACAGCAAAGACGTGGCGATTAAAGGCCAGGGCTTCTTCCAG |
| | | GTGCTGCTGCCGGACGGCAGCCAGGCTTACACCCGCGACGGCTCG |
| | | TTCCAGATCGATCAGAACGGCCAACTGGTGACCGCCAGCGGCTTC |
| | | CAGGTGCAGCCGGCGATCACCATACCGGCCAACGCGTTGACCATT |
| | | ACCATCGCCCGCGATGGCATCGTGAGCGTCACCCAGCAGGGTCA |
| | | GACCGCCGCCCAGCAGGTCGGGCAACTGACGTTAACCACCTTTAT |
| | | TAACGACAGCGGCCTCGAAAGCGTGGGCGAGAACCTGTACCAGG |
| | | AAACTGAAAGCTCCGGCGCGCCGAATGAGAGCACGCCGGGCCTG |
| | | AACGGCGCCGGTATGCTCTACCAGGGTTATGTCGAGACCTCTAAC |
| | | GTCAACGTGGCGGAAGAGCTCGTTAATATGATCCAGACCCAGCG |
| | | CGCCTATGAGATCAACAGCAAAGCGGTTTCGACGTCGGATCAGAT |
| | | GCTGCAGAAACTGACCCAGCTGTAA |
| flagellar | flgK | >ADJCKNHF_03363 Flagellar hook-associated protein 1 |
| hook- | | ATGTCCAATAGCTTAATCAATACGGCGATGAGCGGACTGAATGCG |
| associated | | GCGCAGGTGGCGCTCAGTACCGTCAGCAACAATATCTCTAACTAC |
| protein 1 | | AACGTGGCGGGTTATAACCGGCAGACGGCGATTCTGGCCCAAAA |
| FlgK | | CGGCGGCCTGGCCACCATGAACGGTTTTATCGGTAACGGCGTGAC |
| | | CGTGACCAGCGTGAACCGCGAATATAACCAGTTCATCACCAACCA |
| | | GTTGCGCGGCGCGCAGAGCGCGGCCGGTTCGCAGAACGCCTACT |
| | | ACGAAAAGATTTCGCAGATCGATAATCTGCTGGCCAGCAAAACC |
| | | AATACCCTGTCGGGGAGCCTGCAGGATTTTTTCACCAATTTGCAG |
| | | AACCTGGTGAGCAACGCCGGCGACGACGCGGCGCGCCAGACGGT |
| | | GCTGGGCAAGGCTAACGGCCTGGTTAACCAGTTTAATAATACCGA |
| | | TAAATATCTGCGCGACATGGATAGCGGCGTTAATCAGCAAATTAA |
| | | CGACACCGCCCAGCAGATTAACAGCTATAGCCAGCAGATTGCCC |
| | | GTCTTAACGATGAGATTACCCGCCTGCGCGGCAGCGCCAGCGGCG |
| | | AACCGAACGCGCTGCTCGATCAGCGCGATCAGCTGGTCAGCGAA |
| | | CTCAACCAACTGGTGGGCGTTCAGGTAACCCAGCAGGACGGCGA |
| | | CGCCTATACCGTCTCCTTCGCCAACGGCCTGACCCTGGTGCAGGG |
| | | CAACAACAGCTATCAGGTGGAAGCGATCCCTTCCAGCAGCGACC |
| | | CTTCGCGCCTGACTCTCGGCTATAACCGCGGCAGCGGCGCCAACG |
| | | AAGTGCCGGAAGGGCAGATCACCAGCGGCAGCCTGAGCGGCGTA |
| | | CTGCGTTTTCGCAGCGAGACGCTGGACGGCGTGCGCAATCAGCTC |
| | | GGCCAACTGGCGCTGGCGATGGCCGATAGCTTTAACCAGCAACA |
| | | CCGCGAGGGCTTCGATCTCAACGGTGAGCAGGGGGGCGATTTCTT |
| | | CAGCTTCAGCGGCGCGCGGGTGGTTGATAACACGCGCAATACCG |
| | | GCGATGCCTCGCTGTCGGTGTCGTATACCGATACCAGCAAGGTAA |
| | | AAGCCAACGATTACCGCGTAGAGTATGACGGCGCCAACTGGCAG |
| | | GTCAGCCGTCTGCCGGACAACGTCAAAGTCAACGCCACCGCCGGT |
| | | AAAGATGCAGCCGGTAACCCGACGCTGAGCTTTGATGGTCTCGAA |
| | | GTCAGTATTGATGGCAACCCGCAGCAGAAAGATAGCTTTACGGTG |
| | | AAGCCGGTCAGCGACGTGGCGGGCAACCTGAAAGTGGCGATTAC |
| | | CGATTCAGCGAAAATCGCCGCCGCCGGTAAAGATGACAGCGGCC |
| | | GCGGCGATAACGATAACGCTAAAAAACTGCTCGATCTGCAGACC |
| | | AAAAACCTGGTGGATGGCAAAGCGACGCTTAGCGGCGCCTACGC |
| | | CGGGATCGTCAGCGGCGTGGGCAACCAGACCGCCGCCGCGAAGG |
| | | TGAGCAGCGAATCGCAGTCCAGTATCGTCACCCAACTGGCCCGTC |
| | | AACAGCAGTCGCTCTCCGGGGTAAACCTCGACGAAGAGTATGGC |
| | | GAGCTCCTGCGTTTCCAGCAGTACTACATGGCGAACGCGCAGGTG |
| | | ATCCAGACCGCCAGCTCGCTGTTCGACGCGCTGCTGAATATTCGT |
| | | TGA |
| flagellar | fliR | >ADJCKNHF_03381 Flagellar biosynthetic protein FliR |
| biosynthetic | | GTGATCCCCTTTGACAGCGCCCAGCTTAGCGAATGGCTCAGCCAG |
| protein FliR | | TATTTCTGGCCGCTGCTGCGTATTCTGGCCCTCATCAGCACCGCGC |
| | | CGGTGCTGAGTGAAAAGCAGATAAGCAAAAAGGTGAAAATCGGT |
| | | CTCGGCGGCCTGATTGCGATACTGATCGCCCCAACGCTGCCCATC |
| | | AACGCGATCCCCATTATCTCTGCTGCCGGTTTATGGCTGGCGATTC |
| | | AGCAGATCCTGATCGGCGCGGCGCTGGGGCTAACGATGCAGCTG |
| | | GCTTTCGCCGCGGTACGCCTCGCCGGCGAAGTCATCGGCATGCAG |
| | | ATGGGGCTATCGTTCGCCACCTTCTTCGACCCCAGCGGCGGCCCC |
| | | AATATGCCGGTGCTGGCGCGGCTGCTTAACCTGCTGGCAATGCTG |
| | | CTGTTTTTAAGCTTTGACGGCCACCTGTGGCTGATCTCGCTGCTGG |
| | | CCGACAGTTTCCATACCCTGCCGATCCAGAGCCAGCCGCTGAACG |
| | | GCAACGGTTTCCTCGCGCTCGCCCAACTGGGTTCCTTAATCTTTAT |
| | | CAACGGCATGATGCTGGCGTTACCGCTGATTTGTCTGTTGCTCAC |
| | | GCTCAATATCGCCCTGGGTCTGCTCAACCGTATGACGCCGCAGCT |
| | | ATCGGTATTCGTGATCGGTTTCCCGGTGACCATGACCTTTGGCATT |
| | | ATGACCCTCGGCATGATGATGCCGATGCTGGCCCCCTTCTGCGAA |
| | | CACCTGTTTGGCGAGATGTTCGATCGCCTGGCGGCCGTTATCGGC |
| | | GGTATGACGTTTTAA |
| flagellar | fliQ | >ADJCKNHF_03382 Flagellar biosynthetic protein FliQ |
| biosynthetic | | ATGACGCCGGAATCCGTGATGGCCATTGGCACCGAAGCGATGAA |
| protein FliQ | | GGTCGCCCTGGCGCTGGCCGCCCCGCTGCTGCTGGCGGCGCTAAT |
| | | CAGCGGGCTGGTGGTCAGCCTGCTGCAGGCCGCCACCCAGATTAA |
| | | CGAGATGACCCTATCGTTCATCCCCAAAATTCTCGCGGTAGTGGC |
| | | GACGATTATTATCGCCGGACCGTGGATGCTGAACCTGCTGCTGGA |
| | | CTATATGCGCACATTATTCAGCAACCTGCCTAATATTATTGGCTA |
| | | G |
| flagellar | flip | >ADJCKNHF_03383 Flagellar biosynthetic protein FliP |
| biosynthetic | | ATGTTCCCTACTCTGTTCGCTTGCCTTCGCCGTCGGGCGCCGTGGC |
| protein FliP | | TCGCCGCCGCGTTACTGCTGCTGAGCCCTGCCGCCTTCGCACAGC |
| | | TGCCGGGGCTTATCAGTCAGCCGCTGGCCAACGGCGGCCAGAGCT |
| | | GGTCGCTGCCGGTACAGACGCTGGTGCTGTTGACCTCGCTGACCT |
| | | TCCTGCCGGCGATGCTGCTGATGATGACCAGTTTTACCCGCATCA |
| | | TCATCGTACTGGGTCTGCTGCGTAACGCGCTGGGCACGCCTTCCG |
| | | CGCCGCCGAACCAGGTGATGCTCGGGCTGGCGCTGTTCCTGACCT |
| | | TCTTTATCATGTCGCCGGTGTTCGACAAGGTCTATCAGGAGGCGT |
| | | ACCTGCCCTTCAGCCAGGACAAGATCGGCCTTGATACGGCGCTGG |
| | | ATAAAGGCGCGCAGCCGCTGCGCGAATTTATGCTGCGCCAGACCC |
| | | GCGAAAGCGATCTGGCGCTGTACGCTCGTTTGGCCAACCAGCCGC |
| | | CGCTGGCCGGGCCGGAGGCGGTACCGATGCGTATTCTACTGCCCG |
| | | CCTACGTCACCAGCGAACTGAAAACCTCCTTCCAGATTGGTTTTA |
| | | CGGTGTTTATTCCGTTCCTGATTATCGATTTAGTGGTCGCCAGCGT |
| | | GCTGATGGCGCTGGGGATGATGATGGTGCCGCCGGCGACGATCTC |
| | | GCTGCCGTTTAAGCTAATGCTTTTTGTGCTGGTGGATGGCTGGCA |
| | | GCTGCTGCTGGGCTCGCTGGCGCAGAGCTTCTATTCCTGA |
| flagellar | fliM | >ADJCKNHF_03385 Flagellar motor switch protein FliN |
| motor switch | | ATGAGTGATCCTAAGCAACCGTCTGGCGCAGGAAAGGAATCCGT |
| protein FliM | | AGACGATCTGTGGGCTGATGCGCTTAATGAACAACAGTCGTCTGA |
| | | GAAATCCAGCGCCACCACCGATGGGGTGTTCAAATCGCTGGAGG |
| | | CCCAGGATGCGCTCGGCAGCCTGCAGGATATCGATCTGATCCTCG |
| | | ATATTCCGGTGAAGCTGACCGTTGAACTGGGGCGCACCAAAATG |
| | | ACGATTAAAGAGCTGCTGCGCCTGTCGCAGGGATCGGTTGTCGCG |
| | | CTGGATGGCCTGGCGGGCGAACCGCTGGATATCCTGATCAACGGC |
| | | TATCTGATAGCCCAGGGCGAAGTGGTGGTGGTGGCCGATAAATTC |
| | | GGTGTACGCATTACCGATATCATTACCCCGTCCGAACGTATGCGT |
| | | CGGTTGAGCCGCTAA |
| flagellar | fliM | >ADJCKNHF_03386 Flagellar motor switch protein FliM |
| motor switch | | ATGGGCGATAGCATTCTTTCACAGGCAGAGATCGACGCCTTGCTC |
| protein FliM | | AACGGCGACAACACGGGCGACGAGCCCGAGACGGTTGTCGGCGG |
| | | TAAAGAGAGCGAGGTTAAGCCTTACGATCCGAATACCCAGCGCC |
| | | GCGTGGTGCGTGAACGCCTGCAGGCGCTGGAAATTATCAACGAG |
| | | CGTTTTGCCCGGCAGTTCCGCATGGGATTGTTCAACCTGTTGCGCC |
| | | GCAGCCCGGACATCACCGTCGGGCCGATTAAAATTCAGCCGTATC |
| | | ACGAGTTTGCCCGCAACCTGCCGGTGCCGACCAACCTTAATCTGG |
| | | TACACCTGAACCCGCTGCGCGGCACGGCGCTGTTTGTCTTCGCGC |
| | | CGAGCCTGGTGTTTATCGCCGTCGATAACCTGTTCGGCGGCGACG |
| | | GGCGTTTCCCGACCAAGGTCGAAGGCCGAGAATTCACGCCCACC |
| | | GAACAGCGGGTCATCAAACGCATGCTGCGCCTGGCGCTGGACGC |
| | | CTACGGCGACGCATGGAGCGCAATCTATAAGATTGACGTCGAAT |
| | | ACGTGCGTGCGGAAATGCAGGTCAAATTCACCAACATCACCACCT |
| | | CGCCGAACGATATCGTGGTGACCACGCCGTTCCAGGTGGAGATTG |
| | | GCGCGCTAAGCGGTGAATTCAATATCTGTATTCCTTTCGCCATGA |
| | | TTGAGCCGCTGCGTGAACTGCTGACCAATCCGCCGCTGGAGAATT |
| | | CCCGCCAGGAAGATAGCCAGTGGCGCGAAACGTTGGTCAAGCAG |
| | | GTGCAGCACTCTGAGCTGGAGCTGATTGCCAACTTCGTCGATATC |
| | | CCGATGCGCTTATCGAAAATACTTAAGCTGCAGCCAGGCGACGTT |
| | | TTACCGATAGATAAACCGGATCGCATTATCGCCCATGTCGACGGC |
| | | GTGCCGGTGCTGACCAGCCAATATGGCACCTTAAACGGGCAGTAC |
| | | GCCCTTCGTGTTGAACACTTGATTAACCCTATTTTGAATGCTCTGA |
| | | GTGAGGAACAGCCCAATGAGTGA |
| flagellar | flik | >ADJCKNHF_03388 hypothetical protein |
| hook-length | | ATGAATCTCAATGCTTTGCCCGCCCTTAGCCTACCCGGCGACGCC |
| control | | AGCGCCCTGGCGGATCTGGCGCTCGATGACGCTCAGCTGTCATCT |
| protein Flik | | GCCTTTGCGCAACTGCTTGGCGCGCGCTTCACGCCCGCGCAAAGC |
| | | GGCACGCTGCCGCCGGCGGCGCTGAGCGCCGACGATGAAAGCGC |
| | | CCCGCCGCTAAGCCGTAACCAGCTTAACCAGCTGCTGGCCGCGCT |
| | | TGGCGAACGCGGCACGCTGCTGGGCAATGCGCTACCGGCATCGG |
| | | CGGAAAACGCCGTCGACACAACAGCGAAAAAAGAGGATACGCGC |
| | | CCGGATACGCCACCGCTCAGCGAGGCGAAAACTTTAGATCCGGC |
| | | GACGCTGCAGGCACTGTACGCCATGCTGCCGGCGGCAATCGTCGC |
| | | CCCGCCGCCGCAGACCGCGCTACCGCAGGCTCAACCAGCTGCGA |
| | | CCAGCGGCGATAAAACCGCGCTCCACATCGGCAACGCGGCGACT |
| | | CAGCATGTCGCGAACCTGCCGGAAGCCGAACCGGCGGCGAAGCC |
| | | GCAGCCGGGCACGGATCCCCTTAGCGGGCAAAATCTGCCTTCTCC |
| | | TGTTGTGCAGACCGCGACCACGCCGCAGCACGATATGGCCGAAC |
| | | CTCCGGCGGAGAATACTCCGGCGCAGCCAGCCGCCGTCGCTTTCG |
| | | CCGCCGCCAGCCCCAGCCAGAGCGCCACGCCCGCCAGTGCGCTG |
| | | GTAACCGCGCCGCCGACGCCGCAGCTTAATGCCCAGCTCGGCAGC |
| | | CCGGAATGGCAGCAGGCGCTGAGCCAGCAGGTGCTGATGTTCCA |
| | | CCGCAACGGCCAGCAGAGCGCCGAACTGCGCCTGCATCCGCAAG |
| | | AGCTGGGGGCGCTGCAGATCACCCTGCAGCTCGACGATAAACAG |
| | | GCGCAGCTGCACATCACTTCCGCACACGGTCAGGTACGCGCGGCG |
| | | GTCGAGGCGGCTATGCCGCAGCTACGTCACGCGCTGGCTGAGAG |
| | | CGGAATCAACCTCGGTCAGAGCAGCGTTGGCGGCGAGGCGACGC |
| | | CGCAGTGGCAGCAGCAAAACGGTAACGGTGAAGGCCGCGCCAGC |
| | | TACGCTGAACGCCATGGCGGCGGCAGCGATGCCGCGCCGATTGA |
| | | GCCAGTCAGCGCCCCGGCAGCCCTGCAGCGAATGGCCAACCAGC |
| | | TCAACGGCGTCGATATTTTCGCCTGA |
| flagellar FliJ | fliJ | >ADJCKNHF_03389 Flagellar FliJ protein |
| protein | | ATGAAAGCACAGTCCCCTTTGATTACCCTGCGCAATCTGGCCCAG |
| | | GAGGCCGTCGAACAGGCGGCGCAGCGTTTGGGCCAGGCGCGCCA |
| | | GGCGCAACAGGCCGCCGAGCAACAGCTGACGATGCTGCTGAACT |
| | | ATCAGGACGAGTACCGGCAAAAGCTCAACAGCACCCTTTCTGGC |
| | | GGCATGGAAAGCTCGCGCTGGCAGAACTACCAGCAGTTCATTGCC |
| | | ACCCTCGAACAGGCTATCGAACAACAGCGCCAGCAGCTTCTGCA |
| | | GTGGGGGCAAAAGGTGGATCACGCCGTGAAGCAGTGGCAGGACC |
| | | ATCAGCAGCGGCTGAACGCCTACGATACCCTGCACACGCGCGCG |
| | | CAAAACGCCGAGCTGCAGTTGGAAAACAAACGCAATCAAAAATT |
| | | GATGGATGAGTTCGCGCAACGCAGTACACAAAGGAATACCGCTT |
| | | CATGA |
| flagellum- | fliI | >ADJCKNHF_03390 Flagellum-specific ATP synthase |
| specific ATP | | ATGACCGCCCGTCTCGGCCGCTGGCTGAATTCGCTTGAAGCGCTG |
| synthase | | GAGCAGCGTATCGCCCGAACGCCGACCGTACGACGCTATGGTCGT |
| [EC:7.4.2.8] | | CTGACCCGCGCCACCGGTCTGGTGCTGGAGGCCACCGGCCTGCAG |
| | | CTGCCGCTCGGCGCAACCTGCCTTATCGAGCGCGACGGCGGGGGC |
| | | GGCGTGCAGGAAGTGGAAAGCGAAGTGGTCGGCTTTAACGATCA |
| | | GCGGCTGTATCTGATGCCGCTGGAAGAGGTCGAAGGAATCGTGC |
| | | CAGGGGCGAGAGTTTACGCCCGCAGCGCGGCGGACGGCCAGCAC |
| | | GCCGGTAAACAACTGCCGTTGGGCCCGGCGCTGCTGGGACGCGT |
| | | GCTCGACGGCGGCGCCAGGCCGCTTGATGGCCTGCCCGCGCCGG |
| | | AAACCGGCTATCGCGCGCCGCTGATTACCGCGCCGTTTAACCCGC |
| | | TGCAGCGTACCCCTATCGAGCAGGTGCTGGACGTCGGCGTACGCA |
| | | CCATTAACGGCCTGCTGACCGTTGGGCGCGGCCAACGTATGGGGC |
| | | TGTTCGCCGGCTCCGGCGTCGGTAAAAGCGTGCTGCTGGGCATGA |
| | | TGGCGCGCTACACCCAGGCCGACGTTATCGTCGTCGGCCTGATCG |
| | | GCGAACGCGGCCGGGAAGTCAAAGACTTTATCGAGAATATTCTC |
| | | GGCGCCGAAGGCCGCGCGCGTTCAGTGGTGATCGCCGCCCCGGC |
| | | GGACGTATCGCCGCTGCTGCGCATGCAGGGCGCCGCTTACGCCAC |
| | | CCGCATCGCCGAAGATTTTCGCGATCGCGGCCAGCACGTGCTGCT |
| | | GATCATGGATTCGCTCACCCGCTATGCGATGGCGCAGCGAGAAAT |
| | | CGCGCTGGCGATCGGCGAACCGCCTGCAACCAAAGGCTATCCGC |
| | | CATCGGTATTCGCCAAACTGCCGGCGCTGGTTGAACGCGCGGGCA |
| | | ACGGTATCAGCGGCGGCGGTTCGATCACCGCCTTCTATACCGTAC |
| | | TCACCGAAGGGGACGATCAGCAGGATCCGATCGCCGATTCGGCG |
| | | CGCGCGATCCTCGATGGTCACGTGGTGCTGTCCCGACGTCTGGCG |
| | | GAGGCCGGTCACTACCCGGCCATCGATATTGAAGCCTCGATCAGC |
| | | CGCGCGATGACGTCACTTATCGATGACGAACATTATCGCCGGGTA |
| | | CGCACCTTTAAACAGATGCTGGCCAGCTTCCAGCGCAACCGCGAT |
| | | TTAATAAGCGTCGGCGCCTATGCCGCCGGTAGCGACCCGCTGCTG |
| | | GATAAAGCCATTACCCTGTATCCGCAAATGGAGACCTATCTGCAG |
| | | CAGGGAATTTTCGAGCGCTGCGGTTATGACGAAGCCTGTCTGCAG |
| | | CTACAGCAGCTGATTGTTTAA |
| flagellar | fliH | >ADJCKNHF_03391 Flagellar assembly protein FliH |
| assembly | | ATGTCTGATCGCATTAATTCCATGCCCTGGCAGCCCTGGTCACTC |
| protein FliH | | AACGACCTCGGCGACGCGGCGCCAGCTTTTGAACCGCCGCTACCG |
| | | ACGTTGGATAACGAAGCCTTGCAGCCTGACGCTCAGGCGGAACT |
| | | GCAGCAGCAGCTTGCCGCCCTGCGCCTGCAGGCCGAGCAGCAAG |
| | | GTCAGCAATCCGGTTATGCCGATGGCCAGCAAAAAGGCTATGAA |
| | | GCCGGATTTCAGCAGGGACTTGAGGAGGGCCGCCAGCAGGGGCT |
| | | GCTGGAAGCACAGCAGCAGCAACAGCCGCTGACCGACCACTGGC |
| | | AGCAGCTGGTGAGCGAGTTTCAGCACACCCTTGATGCGCTCGATT |
| | | CGGTCATCGCCTCACGCCTGATGCAGCTGGCGCTAACCGCCGCGA |
| | | AACAGGTGCTTGGCCAGCCGCCGGTCTGCGACGGCACTGCCCTGC |
| | | TGACGCAAATTCAACAGTTGATCCAGCAAGAACCGATGTTCAACG |
| | | GCAAGCCGCAGCTGCGCGTACACCCGCAAGATTATGAGCGCGTT |
| | | GAGCAGCAGTTAGGCACCACCCTCAGCCTGCACGGCTGGCGGTTG |
| | | CTGGCGGATGGCGATCTGCATCCCGGCGGCTGTAAGGTCAGCGCC |
| | | GAGGAGGGCGATCTCGACGCCAGTCTCGCCACCCGCTGGCACGA |
| | | ATTGTGTCGCCTCGCCGCGCCGGGAGATCTGTGA |
| flagellar | fliG | >ADJCKNHF_03392 Flagellar motor switch protein FliG |
| motor switch | | ATGAGCCTGACCGGAACCGATAAAAGCGCCATTTTACTGATGACG |
| protein FliG | | CTGGGCGAAGACCGCGCGGCGGAGGTGCTCAAGCACCTCTCCTCC |
| | | CGCGAAGTCCAGCTTTTGAGCGGCACCATGGCCGGCATGAGCCA |
| | | GGTCTCGCATAAACAGCTCGGCGAGATCCTGTCTGAATTTGAAGA |
| | | CGATGCCGAACAGTTCGCCGCCCTGAGCGTCAACGCCAGCGACTA |
| | | TTTGCGTTCGGTGCTGGTGAAAGCGCTGGGCGAAGAGCGTGCCGC |
| | | CAGCCTGCTGGAGGATATTCTCGAATCCCGCGAAACCACCTCCGG |
| | | GATGGAAACGCTCAACTTTATGGAGCCGCAGAGCGCCGCCGACC |
| | | TGATCCGCGATGAACACCCGCAGATCATCGCCACCATCCTGGTAC |
| | | ATCTCAAGCGCGGCCAGGCGGCGGATATTCTGGCGCAGTTCGATG |
| | | AGCGCCTGCGCAACGACGTAATGCTGCGTATCGCCACCTTCGGCG |
| | | GCGTGCAGCCGGCGGCGCTGGCGGAGTTGACCGAAGTACTCAAC |
| | | AACCTGCTCGACGGCCAGAACCTCAAGCGCAGCAAAATGGGCGG |
| | | AGTGCGTACCGCCGCCGAGATTATCAACCTGATGAAAACCCAGC |
| | | AGGAAGAAGCCGTTATCGACGCGATGCGCGAATACGATGGCGAG |
| | | CTGGCGCAGAAGATCATCGACGAGATGTTCCTGTTCGAAAACCTG |
| | | GTGGAGGTCGACGATCGCAGTATCCAGCGCCTGCTGCAGGACGT |
| | | GGACAGCGAATCGCTGCTGATTGCTCTGAAGGGCGCCGAGCAGC |
| | | CGCTGCGCGAGAAGTTCCTCAAAAACATGTCCCAGCGCGCGGCC |
| | | GATATTCTGCGCGACGATCTCGCCAACCGTGGGCCGGTGCGCATG |
| | | TCGCAGGTGGAAAACGAACAGAAAGCGATTCTGCTGATAGTCAG |
| | | ACGGCTGGCGGAGAGCGGCGAAATGATTATTGGCGGCGGCGAGG |
| | | ACACCTATGTCTGA |
| flagellar M- | fliF | >ADJCKNHF_03393 Flagellar M-ring protein |
| ring protein | | ATGAGTGCCTCGTTATCCGCCGGCGAAAGCCGCGACAACGGCCTG |
| FliF | | CAGGCCATCTGGAGCCGTCTGCGCGCCAATCCAAAAATACCGCTG |
| | | CTGGTGGCCGCCTCCGCCGCTATCGCCATTATCGTCGCGCTGCTG |
| | | CTGTGGGCGAAAAGCCCGGATTATCGCGTGCTGTACAGCAATCTG |
| | | AACGATCGCGACGGCGGCGCTATCGTCACCCAACTGACGCAGAT |
| | | GAACATCCCCTACCGCTTCGCCGAGAATGGCGCGGCGCTGATGAT |
| | | CCCGGCGGAGAAAGTGCATGAAACCCGTCTGCGTCTTGCGCAGC |
| | | AGGGGCTGCCGAAGGGCGGCGCCGTTGGCTTCGAACTGCTGGAT |
| | | CAGGAAAAATTCGGCATCAGCCAGTTCAGCGAACAGATTAACTA |
| | | TCAGCGCGCTCTTGAAGGCGAACTCTCACGCACTATCGAGTCGCT |
| | | GGGGCCGGTGCAGAATGCGCGGGTGCACCTGGCGCTGCCGAAGC |
| | | CCTCGCTGTTCGTCCGCGAGCAAAAGTACCCTTCCGCTTCTGTCAC |
| | | CCTGACGCTGCAGCCGGGCCGTGCGCTGGATGATGGCCAAATCA |
| | | ACGCTATCGTCTATATGGTATCGAGTAGCGTCGCCGGCCTGCCGG |
| | | CTGGCAACGTCACCGTGGTCGATCAGGCCGGACGCCTGCTGACGC |
| | | AGTCTGACGGCACCGGGCGCGACCTCAACGCCTCGCAGCTGAAA |
| | | TATGCCAGCGAAGTGGAAAGCGGCTATCAGCGACGCATTGAAAC |
| | | GATCCTGGCGCCGGTGGTCGGCAGCGCCAACGTCCGCGCTCAGGT |
| | | GACCGCACAGATCGATTTCGCCTCCCGCGAGCAGACCGACGAAC |
| | | AGTACCAGCCGAACCAGCAGCCCGATAAAGCGGCGATCCGTTCG |
| | | CAGCAAACCAGCAGCAACGAGCAAATCGGCGGACCGCAGGTGGG |
| | | CGGCGTTCCTGGGGCGTTATCCAACCAGCCGAGCGCGCCGGCCAC |
| | | CGCGCCGATTGAAACCGCTAAACCGGCGGCCAATAACGCCAGCA |
| | | ACGCGACCAACACCCGTAGCGCCGCGGCCAACAGCGGCCCGTTA |
| | | AGCACCCGACGCGACGCCACCACCAACTACGAACTCGATCGCAC |
| | | CATCCGCCATACCCAACAAAAAGCCGGGACGGTGGAGCGTCTGT |
| | | CGGTCGCCGTGGTGGTGAACTACAGCCTCAACGCCGACGGCAAA |
| | | GCGCAGCCGATGAGTAAAGAACAACTGGCGCAGATTGAAGCTCT |
| | | GGTGCGCGAAGCGATGGGCTACTCCAGCAGCCGCGGCGATAGCC |
| | | TACAGGTGGTCAATACGCCATTTACCGACAATCAGATGACCGGCG |
| | | GCGAGCTGCCGTTCTGGCAGAGCCAGGCGTTTATCGATTTGCTGC |
| | | TCGAACTGGGACGTTATCTGCTGGTGCTGCTGGTCGGCTGGGTAT |
| | | TGTGGCGCAAGCTGATTAAGCCACAGCTGCAGCAGCGTCAGGCC |
| | | GCACAGCAAGCCGCGGTCGCCGCCGCCAGCGCGCCGGTCGCTAA |
| | | AGCGAACGACAGCCATAAACCAAGCAATGAGGAGCTGGCGCTGC |
| | | GCCGTAAATCGCAGCAGCGCGTCAGCGCAGAAGTCCAGAGCCAG |
| | | CGCATCCGCGAACTGGCGGATAAAGATCCGCGGGTCGTCGCTCTG |
| | | GTAATCCGCCAATGGATGAGTAACGAACTATGA |
| flagellar | fliE | >ADJCKNHF_03394 Flagellar hook-basal body complex protein |
| hook-basal | | FliE |
| body | | ATGGCGATTCAGGGTATTGAAGGCGTACTGCAACAGATGCAGGC |
| complex | | GATGGCGATTCAGGCGGGAAATAACGGGCAGAATAGCGTACCGC |
| protein FliE | | AGGGAGTCAGTTTTGCCAGTGAATTGACCGCCGCGCTGGGTAAAA |
| | | TCAGCGATACCCAGCAGGCCGCGCGTCAACAGGCGCAAAACTTC |
| | | GAGCTGGGCGTGCCGGGTATCAGCCTGAATGATGTGATGGTCGAT |
| | | TTACAGAAATCTTCAGTTTCGTTGCAGATGGGCGTGCAGGTGCGC |
| | | AATAAGCTGGTGGCGGCCTATCAGGACATTATGAATATGCCGGTT |
| | | TAG |
| flagellar | fliT | >ADJCKNHF_03398 Flagellar protein FliT |
| protein FliT | | ATGGAACGCCAACAGCAGCTTTTAGCCGCCTATCAGCAGATCCAT |
| | | ACCCTGAGCAGCCAGATGATTGCCCTGGCGCGCGCCGGGCAATG |
| | | GGAAGCGCTGGTGGAAATGCAGTTTGCCTACGTGACGGCGGTCG |
| | | AGAAAACCGCCGAATTTACCGGCAAAGCGGGGCCATCGCTGGCT |
| | | CTGCAAGAGATGCTCAACGCTAAGTTGAAGCAGATTATCGACAAT |
| | | GAGGGAGTACTGAAAGGGCTATTGCAACAGCGGATGGAACAATT |
| | | AAAAACGCTGATCGATCAATCCACCCGCCAGAACGCGGTCAACA |
| | | ACGCTTACGGCCAGTTCGACGATCGTTCGCTCCTGCTTGGCGAAC |
| | | TGCAGTCCGATAACGTGGTCGCCATTAAGAGCAAAAGCGAAGAA |
| | | TTACAATAA |
| flagellar | flis | >ADJCKNHF_03399 Flagellar secretion chaperone FliS |
| protein FliS | | ATGTATAACCGTAGCGGCACGCAAGCCTACGCACAGGTCAGTCTG |
| | | GAAAGCAGCGCGATGAGCGCCAGTCCGCACCAGTTAATCGTAAT |
| | | GTTGTTCGACGGGGCGCTTAACGCCCTGCTTCGCGCACGCATCCT |
| | | GATGAATCAGGGGGATATCGCCGGTAAAGGCCTGGCGCTGTCGA |
| | | AGGCAATCAACATCATCGACAACGGGTTGAAAAGCGGCCTCGAC |
| | | CACCAGCAAGGCGGCGAAATCGCCGATAATCTGGCGGCGCTGTA |
| | | CGATTATATGAAGCGACGCCTAATGCAGGCCAACCTGCATAATGA |
| | | CGAAGCGGCGATCGTCGAAGTGGTCAAGCTGCTGGAGAATATCG |
| | | CCGATGCCTGGCGACAAATTGGGCCAAACTATCAACCTGCTCAGG |
| | | GTACATTGTGA |
| flagellar | fliD | >ADJCKNHF_03400 Flagellar hook-associated protein 2 |
| hook- | | ATGGCATCGATCAGTTCCTTAGGCATCGGCTCAGGACTCGACCTC |
| associated | | AACGGTTTACTGGACAAACTCAGCAAGGCGGAACAACAGCGTCT |
| protein 2 | | GACGCCGTATACCACTCAGCAAACCAGCTATAACGCGCAGCTAA |
| | | CGGCTTACGGCACGTTAAAAAGCTCGCTGGAAAAATTCGATAACC |
| | | TGAGCAAGGAGCTGGCTAAGCCGGAGTTTTTCAACAGCACCACC |
| | | GCCACCAAGCACGACCAGTTCACCATCACCACCACCAATAAGGC |
| | | GGTGCCGGGCAACTATAGCGTTGAAGTGCAGAAGCTGGCGCAGC |
| | | CGCAAACCCTGACCACCCAGGCGACAATTACCGACCAACAAGAG |
| | | AAACTTGGAACCGCCGGCAACAGCGACCGCTCTATTACGATCACC |
| | | GCCGGCGACCCGCCAAAAGAAACCACGATTCCGCTGAGCGACGA |
| | | CCAGACCTCGCTGCTGGAGATGCGCGATGCCATCAACGGCGCTAA |
| | | AGCCGGCGTCACCGCCAGCATCATGCGCGTCGGCGACAATGACT |
| | | ATCAGCTGGCGCTGAGCTCCTCCACCACCGGCGAAAAAAACACC |
| | | GTGTCGGTGCAGGTCAATAACGACGATAAACTCGGCGCTATCCTC |
| | | AACTACGACAGCAATAGCAAAAGCGGCGCGATGAAGCAGACCGT |
| | | TGCCGGACAGGATGCGGAGATTATCGTCAACGGCACCAAAATTA |
| | | AGCGCAGCACCAACTCGATTGCCGATGCGCTGCAGGGCGTCACC |
| | | ATCGACCTGAAAACCACCACCAAAGCTGGCGAACCGCAAAACCT |
| | | GGTGATCGGCATCGATAAAAGCGGCACCGCCGACAAAATCAAAG |
| | | AGTGGGTGGATAATTATAATTCGCTGCTCGACACCTTCGGCTCGC |
| | | TGACCAAATACACCCCGGTGAAAAGCGGTGAAGCGCAAAGCGCC |
| | | AAGAACGGCGCGCTGCTGGGCGATAACACCCTGCGCGGCATTCA |
| | | GTCATCGATTAAAAGCGCGCTCAGCGCCGCTCAGGACAACCCGG |
| | | AGCTGAAAGGCCTTGGCAACCTCGGGATCTCAACCAATCCCAAA |
| | | ACCGGCAAGCTTGAAGTCGACAGCACCAAGCTCAATAAAGCGCT |
| | | CGACGAGAAACCGGATCAGGTCGCCAACTTCTTCGCCGGCAACG |
| | | GCAAAGACACCGGGATGGCCACCGAAATCCATAATGAAATCCAG |
| | | AACTATATTAAAGCGGGCGGCATCATCGAGAACTCGACCAAAAG |
| | | CATCAACACCAATCTCGATCGCCTGAATATCCAGATCGACACTGT |
| | | TTCCGACAGCATCCAGAACACTATCGATCGCTATAAACAACAGTT |
| | | TGTTCAGTTGGATACCATGATGTCCAAGCTGAGCAGCACGGGTAA |
| | | CTACTTACAACAGCAGTTCTCGTCCCAGTAA |
| flagellar | flgL | >ADJCKNHF_03401 Flagellin |
| hook- | | ATGGCACAAGTAATTAATACCAACAGCCTGTCGCTGATGGCGCAA |
| associated | | AACAACCTGAACAAATCCCAGTCTTCCCTGGGCACGGCGATTGAG |
| protein 3 | | CGTCTGTCTTCCGGTCTGCGCATCAACAGCGCCAAGGACGATGCC |
| FlgL | | GCGGGTCAGGCGATCTCCAACCGTTTTACCGCTAACATCAAAGGC |
| | | CTGACTCAGGCTTCCCGTAACGCTAACGACGGTATCTCCCTGGCG |
| | | CAGACCACCGAAGGCGCGCTGAATGAAGTCAACGATAACCTGCA |
| | | GAACATCCGTCGTCTGACTGTGCAGGCGCAGAACGGTTCTAACTC |
| | | TTCCAGCGACCTGCAGTCCATCCAGGATGAAATCACCCAGCGTCT |
| | | GTCTGAAATTGACCGAATTTCTCAGCAAACCGATTTCAACGGTGT |
| | | GAAAGTACTGAGTAGCAATCAGAAGCTGACTATTCAGGTTGGCG |
| | | CTAATGATAATGAAACGATTGACATCGACCTGAAAGATATCAATA |
| | | AAAGCACTCTAAATTTAGATACCTTCTCTGTGGCATTCGGCGTCA |
| | | ATAAGGGGGATGTCGGTAAAACTGCTGTTGCTGCAAATGATTCCA |
| | | TTCAACTGGATAAAACAGTAACAATCGCTGCCGGGCAAAAAGCA |
| | | AATTCTAAAGACGTTACTGGTTATGTTAAAGATGATAAGGGTGAT |
| | | ATTTATGCTCGTACTGGCGCTACAGAGTATTACAAAGTAGAAAGC |
| | | ATTGACAAAGATGGAACAGCAACTTTTGCAGCGGCACCGGCAGC |
| | | ACCATCTGGTACGACTAAAGCAGTAACCGAAGGTAAGATTTCTGT |
| | | TAAGGCAACCGATAATGCAGATAAAGATTTAGCTAATACTTTCGC |
| | | GTACACGGGTACAGAGAAAGGTGCTCCTAAATATGTGATTAAAG |
| | | ATACTAGTGGCGCGACTGACCGTTATTACGCGGCTACTTTTGATG |
| | | CCGATGGTAAAGTGGTTCGCGGCAACGAGCTCAGTTCAACTCCTG |
| | | CTACTGAAGATCCGCTTGAAGCATTAGACAAAGCCCTGTCCCAGG |
| | | TAGACCAACTGCGCTCCTCCCTGGGTGCGGTACAGAACCGTTTCG |
| | | ATTCTGTTATCAACAACCTGAACAGCACCGTGAACAACCTGTCCG |
| | | CGTCCCAGTCCCGTATTCAGGATGCTGACTACGCGACCGAAGTGT |
| | | CCAACATGAGCCGCGCGAACATCCTGCAGCAGGCGGGTACCTCC |
| | | GTGCTGGCGCAGGCTAACCAGTCGACTCAGAACGTCCTGTCTCTG |
| | | CTGCGTTAA |
| ferredoxin- | nasB | >ADJCKNHF_03579 Nitric oxide reductase FIRd-NAD(+) reductase |
| nitrate | | ATGCGGCGACTGGTAATTATCGGCAACGGTATGGCGGCAACCCG |
| reductase | | GCTGGCAGAGAAGCTGGCGGCGCGCGCGCAGGGGCGTTTCATCA |
| [EC:1.7.7.2] | | TCACGCTACTCGGCGATGAACCGCAGCCGGCCTACAACCGCATTC |
| | | AGCTCTCGCCGGTACTCGGCGGGGAAAAAACGGCGTCGCAAATC |
| | | CAACTGCTGCCGACGGAGTGGTACGCCCAGCATGGTGTCCGCGTG |
| | | CGGCTTGGCGAGGCGGTCAGCGAAGTGGATGTCGCGGGGCGGAG |
| | | GCTGCGCGTCGGCGGCGACTGGCTGGAATGGGATGAACTGGTGTT |
| | | CGCCACCGGGTCGCAGCCGTTTATCCCGTCGCTGCCGGGTATTGA |
| | | GCGCCCGCAGGTATTCGCCTTTCGCACCCTGGCGGATGTTAAAGA |
| | | CATTCTGGCAATCCCCGGCCCGGCGGTGGTAATCGGCGGTGGCGT |
| | | ACTGGGCGTGGAGGCCGCGGCGGCGCTGCGCCGTCACGGTGGGG |
| | | AAGTGACGCTACTCCATCGCGGTAGCGGTTTGATGGAGCCGCTTA |
| | | CCGATGCTTTTGCCGCGGAACAGTTAAAACAACAGCTTGAGGCAC |
| | | GCGGCATCCGCTGCGAGCTGGAGAGCCGAATTGCCGCTATTGACG |
| | | AGCAGCATGTGCTGCTGGAAGACGGGCGCGCTTTTGCCGCCAGCC |
| | | GGGTGGTGTTGGCCACCGGCGTGCGGCCGGATATCCGTCTGGCGA |
| | | CGCGCAGCGGCGTAGCCTGCCAGCGGGGGATTATCGTCGACCGC |
| | | CGGATGGCGACCTCGCTGCCGGGGATCAGCGCTATCGGCGAGTGT |
| | | TGTGAGATTGACGGCCAAACCTGGGGACTGGTGGCTCCCTGCTTG |
| | | CGCCAGGCCGATGTATTAGTTGAACGCCTGTGCGGGGCTCCCGGC |
| | | GAAGAATTCAGCTGGCAGGACGGCGGCACCCGGCTGAAGGTCAC |
| | | CGGCATCGACTTTTTTAGCGCCGGCGAACAGCGGGCCGGCGAGC |
| | | AGGACCAGATCTACAGCAGTTGGGACCCCATCGATCGCCACTACC |
| | | GTCGCCTGCTGATCCGCGATGGCAGGTTGCACGGCGTGCTGCTGC |
| | | TGGGCGACTGCCAGCAGGCGGCGCCGCTTACCGCGCTGCTCGGCA |
| | | GCGATAGGTCCCCTTCAGTGGAGTGGCTTTTTGACCCTTCTATAAC |
| | | GCAGCCGCGGGCTGCAGGAAAAATGACGATGACTAAACCTGTAT |
| | | TAGTGCTGGTCGGACACGGCATGGTCGGCCACCATTTTCTTGAGC |
| | | AATGCGTGAGCCGGAACCTGCACCAGCAGTACCGTATCGTGGTTT |
| | | TTGGCGAAGAGCGCTATGCCGCCTATGACCGGGTTCATCTCTCGG |
| | | AATATTTCGCCGGGCGCAGCGCAGAGTCGCTATCGCTGGTAGAGG |
| | | GAGACTTTTTTAGCCAGTACGGCATTGAGCTGCGGCCAGGAGAGA |
| | | CGATCGCGGAAATCGATCGTCCGGCGCGAATCGTGCGCGATGCCC |
| | | ACGGCCATGAAACCCATTGGGATAAGCTGGTGCTGGCGACCGGC |
| | | TCCTATCCCTTCGTGCCGCCAATCCCCGGTAACGATGAAGAAGGC |
| | | TGCTTCGTCTATCGTACGCTTGACGATCTTGACCGCATTGCCGCCC |
| | | GCGCCGCCACTGCGCAGAGCGGGGTGGTCATTGGAGGCGGGCTG |
| | | TTGGGGCTGGAAGCCGCCAACGCGTTAAAACAGTTGGGGCTTAA |
| | | GACCCATGTGGTGGAGTTCGCCGCCAACCTGATGGCGGTGCAGCT |
| | | GGATAACGGCGGCGCGGCGATGCTGCGGGAGAAAATTCTCGATC |
| | | TTGAGGTTGGCGTGCACACCAGCAAAGCGACGACGGCGATAGTG |
| | | CGCACCGCCGACGGGCTGCAGCTTAACTTCGCCGATGGCGGCACG |
| | | CTGCAGACCGATATGGTGGTCTTTTCCGCCGGGATCCGCCCGCAG |
| | | GATGCGCTGGCGCGCGGCGCAGGCCTGGCAATCGGCGAACGCGG |
| | | CGGTATCGGTATCGATGACCACTGCCGGACCTCGGACGCAGATAT |
| | | ATTGGCGATTGGCGAATGCGCGCTGTGGGACAACAAAATTTATGG |
| | | TCTGGTAGCGCCGGGCTACCACATGGCGCGCACCGCGGCGGCGC |
| | | AGTTGGCTGGTGAAGCGGCCCGCTTTAGCGGCGCCGATATGAGCA |
| | | CCAAACTCAAACTGCTCGGCGTGGATGTAGCGTCGTTTGGCGATG |
| | | CTCACGGACGTACAGCTGGTTGCCAGAGCTATCAATGGACCGATG |
| | | GTCCGCGGCAGATCTACAAAAAGATCGTTGTCAGCGCTGATGGCA |
| | | AAACGCTGCTGGGCGGCGTATTGGTGGGCGATGCCGGTGACTAC |
| | | GCGACGCTGTTGCAGATGATGCTGAACGGCATGGCGCTACCGTCT |
| | | CGTCCGGAAAGTCTGATCCTCCCGGCCATGGATGGGGCCACGACG |
| | | AAAGCGCTGGGGGTGGCGGCGCTGCCGGATAGCGCGCAGATTTG |
| | | CTCTTGCCATAACGTCAGTAAGGGAGACATTTGCCAGGCGGTCAG |
| | | CGCCGGAGCGGGCGATATGGCGGCGATAAAAAGCTGCACTAAAG |
| | | CGGCGACCGGCTGCGGAGGCTGTAGCGCGCTGGTCAAACAGGTG |
| | | ATGGAACACCAGCTTACCGGGCAGGGCGTCGAGGTTAAAAAAGA |
| | | TATCTGCGAGCATTTTCCGTGGTCGCGACAGGAGATCTATCATCT |
| | | GGTACGAGTCAACCACATCCATACCTTCGATCAGCTCATTAGCCG |
| | | TTACGGGCAGGGCCACGGCTGTGAAGTGTGTAAACCGCTGGTGG |
| | | CGTCGGTACTGGCCTCCTGCTGGAATGAGTATCTGCTGAAACCGG |
| | | CGCATTTGCCGCTGCAGGACACCAACGATCGCTACTTCGCCAATA |
| | | TTCAGAAAGACGGCACTTACTCGGTGGTGCCGCGGATGGCCGCA |
| | | GGCGAAGTGACACCGGATGGGCTTATCGCTATCGGCCAGATCGCC |
| | | AAACGTTACCAGCTGTACAGTAAAGTGACCGGAGGACAGCGTAT |
| | | CGACCTGTTTGGCGCGCGGCTGGAGCAACTGCCGGACATCTGGCG |
| | | CGAACTGGCGGCGGCGGGATTTGAAACCGGACACGCCTACGGCA |
| | | AATCATTACGCACGGTAAAATCCTGCGTCGGCTCGACCTGGTGCC |
| | | GCTATGGGGTTCAGGATTCAACGGGGCTGGCGGTAAGGCTTGAA |
| | | CATCGTTACAAGGGGCTGCGCGCGCCGCATAAAATCAAAATGGC |
| | | GGTCTCGGGCTGTACCCGCGAATGTGCCGAAGCGCAAGGTAAAG |
| | | ACATTGGGGTTATTGCGACTGAAAAGGGCTGGAATCTTTACGTCT |
| | | GCGGCAACGGCGGGATGAAACCGCGCCATGCGGACCTCTTTGCG |
| | | AGTGACCTTGATGACGCCAGCCTAATCCGTACCGTCGATCGGTTG |
| | | TTGATGTTTTATATTCGCACCGCCGACCGCCTGCAGCGCACCAGT |
| | | AGCTGGATGGATAACCTTGAAGGCGGCGTGGCGTATCTACGCGA |
| | | GGTGATCTTGCACGATAGTCTCGGTATCGGCGATGAGCTGGAGCA |
| | | GGAGATGGCGCGGGTGGTGGATAGCTACCAGTGCGAATGGCAGA |
| | | CCACCCTGAACGACCCACAGCGGCTGGCGCTATTTCGTTCCTACG |
| | | TTAACAGCGATGAGGCCGATGAGGCGGTGCAGCGGCAAATCCTG |
| | | CGCGGTCAGCCGCAGCTGGTGGCGCCGCAAGGGATTGGGCAACC |
| | | GGCCCTGCCGTCGCGGCCGTGGCAGGCGATCTGCGATCTGGACGC |
| | | GATCCCGCCGCAGGCGGGGATCGGCGCGCGCCTCGGCGAGCGGC |
| | | AGATTGCGCTGTTCCGCTTCGGCGAGCAAGTTTATGCGCTGGATA |
| | | ACCAGGAACCCGGCAGCGAGGCCAACGTGTTATCGCGCGGCATT |
| | | TTAGGCGACGCGGGCGGCGAGCCGATCGTTATTTCGCCGCTGTAT |
| | | AAACAGCGGATTCGCTTGCGAGATGGTCGCTTGTACGACAGCGGC |
| | | GAGCCGTCGGTGCGCGCGTGGCCGGTGAAGATCGAACAGGGCAA |
| | | AGTGTGGGTCGGCAACCAGGCATTGCTGCTGCGCGCGGAGGCGA |
| | | GCTGA |
| ferredoxin- | nasB | >ADJCKNHF_03580 Nitrate reductase |
| nitrate | | ATGAGCGAAACCCGAACCACCTGTCCATACTGCGGCGTCGGCTGC |
| reductase | | GGAGTTATCGCCCGCGTTGAACCCAGTGGAACCGTCACGGTTCGC |
| [EC:1.7.7.2] | | GGCGATGAAAACCATCCCGCTAACTTTGGCCGCTTGTGCGTAAAA |
| | | GGGGCAGCGCTTGGGGAAACGACCTCGCTTAGCGGGCGGTTATT |
| | | ACACCCTGAAGTCGATGGCCGGGCGGTCGGCTGGCCGCAGGCGC |
| | | TGGCGGAGGCGGGTTCACGTCTGCGCGACATTATTGAGCGCTACG |
| | | GCCCGCAGGCGGTGGCGTTTTACGCTTCCGGTCAGCTGTTAACTG |
| | | AGGATTATTACGCCGCCAACAAGTTGATGAAGGGCTTTATCGGCG |
| | | CCGCCAATATCGATACGAATTCGCGGTTATGTATGTCATCGGCGG |
| | | TCACCGGCTATAAGCGGGCGCTCGGCGCGGACGTCGTGCCGTGTA |
| | | GCTATGAAGATGTCGAGCAGAGCGATCTGGTGGTGCTGGTGGGCT |
| | | CCAACGCCGCCTGGGCGCATCCGGTTCTCTATCAGCGGTTGGTGC |
| | | AGGCGAAACGCGATAATCCGCGGTTGCGGATCGTGGTGGTGGAT |
| | | CCGCGACGCACCGCGACCTGTGATATTGCCGACGATCATCTGGCG |
| | | TTGGCGCCCGGTAGCGATGGCGGGCTATTTGTCGGCCTGCTAAAT |
| | | GCCATTGCCGCCAGCGGTGGAATGGCGGCGGGTTTTCGCGACCAG |
| | | CGGCAGGCGCTGACCATCGCCGCGCAGTGGAGCGTGGAAAAAGC |
| | | GGCCGCTTTCTGCGGCCTGGCGCCGGAGCAAGTGGCTCGCTTTTA |
| | | TCGTGATTTTATCGCTGCACCGCGCGCCATCACGCTGTACACCAT |
| | | GGGAATTAATCAGTCCGCCAGCGGCAGCGATAAGTGCAACGCGA |
| | | TTATCAATGTCCATCTTGCCAGCGGGAAGTTTGCCCGCCGCGGCT |
| | | GCGGGCCGTTTTCGCTGACCGGTCAACCCAACGCCATGGGCGGGC |
| | | GTGAAGTGGGGGGGCTGGCGACGATGCTGGCGGCGCATATGAAT |
| | | TTTGAGCCGCAGGACCTGCAGCGATTAGCGCGCTTTTGGGGCAGC |
| | | GAGCGACTGGCGCAGACGCCGGGACTAACCGCCGTCGAACTCTT |
| | | CGCCGCGATTGGACGCGGCGAGGTCAAAGCGGTGTGGATTATGG |
| | | GGACCAATCCGGTGGTCTCACTGCCCGATAGCCACGCGGTGAGCC |
| | | AGGCGCTGTCCAGGTGTCCGCTGGTGATGGTCTCCGATGTCGCCG |
| | | CCAATACCGATACGGGGCGCTTTGCCCATATCCGCTTTCCGGCGC |
| | | TGGCATGGGGCGAAAAAAACGGTACCGTGACCAACTCGGAGCGG |
| | | CGGATTTCGCGTCAGCGGGCTTTTTTGCCGCCGCCGGGGGAGGCG |
| | | AAAGCCGACTGGTGGATCATTTCCCGTCTCGCCGCCGAATTAGGT |
| | | TATGCCCGAGCTTTTTCATGGCAACATCCGCATGAGGTATTTTGTG |
| | | AACACGCGGCGCTCTCAGGGTTTGAAAATGACGGTCAGCGGGCG |
| | | TTTGATATTGGCGGGCTGGCGGACCTCAGCCGGGAGGCGTGGGAT |
| | | GCCCTGACACCGGTGCGCTGGCCGGTGAGCCGCAGCGCCGCGCC |
| | | CTGGCGGTTAACGGAGGGCTGGCACGGCGACGGCAGGTTGCGGA |
| | | TGGTGCCGGTGTCGCCGCAGCCGACCCGGGCGCAGACCGAGGCA |
| | | TTTTATCCATTAATCCTCAATAGCGGACGTATTCGCGATCAGTGG |
| | | CATACCATGACCCGCACCGGCGAAATACCGCGGTTGATGCAGCA |
| | | CATTGCCGAACCAGTCGTCGAGGTGGCGCCTGCCGATGCGCGTCG |
| | | TTTTCAATTACGTGAGGGAGAACTGGCTCGCGTCTGGTCGCGCCA |
| | | CGGCGTGATGATTGCGAAGGTGATAGTCAGCGAAGGGCAGCGCG |
| | | CCGGTTCGCTCTTTGTGCCGATGCACTGGAATAATCAGTTTGCCC |
| | | GCCAGGGACGGGTCAATAATTTACTGGCGGCGGTCACCGATCCGC |
| | | ATTCCGGGCAGCCGGAGAGCAAGCAGTCGGCGGTGGCGATAGCG |
| | | CCGTGGCGGCCGGCGTGGCAAGGGGAGCTATTATCTCGCGAGCC |
| | | CTTAGCGTTGCCATCCTCGCTGCACTGGCGGCGACGGGCGGTGGT |
| | | AGGGCTTAGCCATTTGTCGCTGGCCGCGGATACCGGCCCGCAAAG |
| | | CTGGCTGACCGAGTGGTGTCGCAAGCAGGGATGGCAGTTGCAAA |
| | | TTGCCGAGGGCGGCGGGCTGTGGAACCTACTGGCATGGCACGAC |
| | | GGGCAATTGATGCTCGGCTGCTGGAGCGATATTAAGCCGCCGCAA |
| | | ATCGATGCTGAATGGCTGTATACGGCGTTTCGGACGCCGCCGCAG |
| | | ACCGCCGGCCTACGCCATGCGTTGCTCAGCGGCCGTAAGGGTGGC |
| | | GACAGCGCGCCGCGTGGGCAGACTATCTGTAGTTGCTTTAGTATC |
| | | GGCGAGCGGCAGATCGGCGAGGCTATTGCCGGCGGCTGCGTCAG |
| | | CGTAGAGGCGCTTGGCGCGAAGCTCAAGTGCGGCACCAACTGTG |
| | | GTTCCTGTATTCCGGAACTCAAAGCGCTGCTGGCGGCAAATCAAA |
| | | CCCGAACGCCGGTTTGA |
| MFS | narK, nasA, | >ADJCKNHF_03584 Nitrate/nitrite transporter NarK |
| transporter, | NRT, nrtP | ATGAGTCAATCTTCAATCCCGGAGAAGGCTAGCAGCTCAGTCATC |
| NNP family, | | ACCGACTGGCGTCCTGAAGATCCTGAGTTTTGGCAACAGCGCGGC |
| nitrate/nitrite | | CACCGTGTGGCGAGCCGCAATTTATGGATCTCCGTTCCCTGTCTTT |
| transporter | | TACTGGCGTTCTGCGTCTGGATGTTGTTTAGCGCCGTCGCGGTAA |
| | | ACCTCAATAAAGTGGGTTTTCAGTTCACTACCGATCAGCTGTTTAT |
| | | GTTGACCGCATTGCCAGCGCTTTCCGGCGCGCTGCTGCGCGTACC |
| | | TTATGCCTTTATGGTCCCGCTGTTCGGCGGCCGCCGCTGGACGGC |
| | | GTTCAGTACCGGTATCATGATTATTCCGTGCGTTTGGCTGGGCTTT |
| | | GCGGTACAGGATACTTCCACGCCGTTTAGCGTGTTCGTGATTATC |
| | | TCTCTGCTGTGCGGCTTTGCCGGCGCTAACTTTGCTTCCAGTATGG |
| | | CGAACATCAGCTTCTTCTTCCCGAAAGAGAAGCAGGGCGGCGCG |
| | | CTGGGGGTTAACGGCGGCCTTGGTAACATGGGCGTCAGCGTGATG |
| | | CAGCTGGTTGCGCCGCTGGTAGTCTCGATTTCTATTTTTGCCGTTT |
| | | TTGGCGGTAGCGGTAGCGAGCAGCCGGATGGCTCAATGCTGTATC |
| | | TGGAAAACGCCGCGTGGATCTGGGTGCCATTCCTGATCATCTTTA |
| | | CCCTGGCCGCGTGGTTCTTTATGAACGATCTGTCGGCCTCAAAAG |
| | | CGTCGCTGAGCGAACAGTTGCCGGTACTTAAACGCCTGCATCTGT |
| | | GGATTATGGCGCTGCTGTATCTGGCGACCTTTGGTTCCTTTATCGG |
| | | TTTCTCCGCCGGGTTTGCGATGCTGTCAAAAACCCAGTTCCCGGA |
| | | CGTGCAGATCCTGCACTATGCCTTCTTCGGTCCGTTTATTGGCGCG |
| | | CTGGCGCGTTCGATGGGCGGGGCGATTTCCGACCGTCTCGGCGGG |
| | | ACCCGCGTGACGCTGGTGAACTTCGTGGTGATGGCTATTTTCTGC |
| | | GCGTTACTGTTCCTGACCCTGCCAACCAATGGTCAGGGCGGCAAC |
| | | TTCATCGCCTTCTTCGCGGTATTTATGGTGCTGTTCCTGACCGCCG |
| | | GGCTGGGCAGCGCCTCTACCTTCCAGATGATTTCCGTGATCTTCC |
| | | GTAAGCTTACGATGGACCGCGTGAAAGCGCAGGGCGGCAGCGAA |
| | | GCGCAAGCGATGCGTGAAGCGGCGACGGATACCGCCGCGGCGTT |
| | | GGGCTTTATTTCGGCGATTGGCGCGATCGGTGGTTTCTTTATTCCG |
| | | AAAGCGTTCGGTATTTCGCTGGATCTGACCGGCTCGCCGGCCGGG |
| | | GCAATGAAAGTCTTCCTCGTTTTCTATATTGCCTGCGTAGTAATCA |
| | | CCTGGGCGGTATACGGCCGTAAACAGAAATAA |
| nitrate | nasC | >ADJCKNHF_03585 Respiratory nitrate reductase 1 alpha chain |
| reductase, | | ATGAGTAAATTTCTGGACCGGTTTCGCTACTTCAAACAGAAGGGT |
| catalytic | | GAAACCTTTGCCGATGGGCACGGCCAGCTTCTTAAAACCAACCGG |
| subunit | | GATTGGGAGGATGGATACCGCCAGCGTTGGCAGCATGACAAAAT |
| | | CGTGCGTTCCACTCACGGTGTGAACTGCACCGGCTCATGCAGCTG |
| | | GAAAATTTATGTCAAAAATGGCCTGGTCACCTGGGAAACCCAGC |
| | | AAACTGATTACCCGCGTACCCGCCCCGATCTGCCGAACCATGAAC |
| | | CACGCGGCTGCCCGCGCGGCGCCAGCTACTCCTGGTATCTGTACA |
| | | GCGCTAACCGCCTGAAATATCCGCTGATGCGTAAGCGTCTGATGA |
| | | AGATGTGGCGTGAAGCGAAAGTTCAGCATAGCGATCCGGTTGAG |
| | | GCATGGGCTTCAATTATTGAAGATGCCGATAAAGCGAAAAGCTTT |
| | | AAACAGGCCCGCGGTCGCGGCGGTTTTGTTCGTTCTTCATGGAAA |
| | | GAAGTGAACGAACTGATTGCCGCTTCAAACGTCTATACCGTCAAA |
| | | ACTTACGGTCCAGACCGCGTGGCAGGCTTTTCGCCGATCCCGGCG |
| | | ATGTCGATGGTTTCCTATGCGTCCGGCGCCCGTTACCTGTCGCTGA |
| | | TTGGCGGTACCTGTCTGAGCTTCTACGACTGGTACTGCGACCTGC |
| | | CGCCAGCCTCGCCGATGACCTGGGGCGAACAGACCGATGTGCCG |
| | | GAATCTGCCGACTGGTATAACTCCAGCTACATCATCGCCTGGGGC |
| | | TCCAACGTCCCGCAGACCCGTACCCCGGACGCGCATTTCTTTACC |
| | | GAAGTTCGCTACAAAGGGACCAAAACCGTGGCGATTACTCCGGA |
| | | CTATGCCGAAATCGCCAAGCTCTGCGATCTGTGGCTGGCGCCGAA |
| | | GCAGGGTACCGATGCCGCGATGGCGCTGGCGATGGGCCACGTCA |
| | | TGCTGCGTGAGTTCCACCTCGATAAACCGAGCCAGTACTTCACCG |
| | | ATTACGTACGTCGCTACACCGACATGCCGATGCTGGTCATGCTCG |
| | | AAGAGCGCGACGGCTACTATGCCGCTGGCCGTATGCTGCGCGCTG |
| | | CCGACCTGGTTGACTCCCTGGGCCAGGAGCAGAATCCGGAATGG |
| | | AAAACCGTCGCCTTTGACGAGAAGGGCGAAATGACCGTACCTAA |
| | | CGGTTCTCTGGGTTTCCGCTGGGGCGACAAAGGCAAGTGGAACCT |
| | | CGAACAGCGCGATGGTAAAACCGGCGAAGAAATTGAGCTGCGCC |
| | | TGAGCCTGCTTGGCAGCCATGATGATATCGCCAACGTCGGTTTCC |
| | | CATACTTTGGCGGCGAAGGTTCCGAGCATTTCAACAAAGTCGACC |
| | | TCGAAAACATTCTGCTGCACAAACTGCCGGTAAAACGCCTGCAGA |
| | | TGGCCGATGGTTCCACCGCGCTGGTGACTACCGTTTATGACCTGA |
| | | CCATGGCGAACTACGGCCTGGAACGCGGTCTGAACGATGAAAAC |
| | | TGCGCGACCAGCTACGATGACGTCAAGGCGTACACTCCGGCCTGG |
| | | GCAGAAAAAATCACCGGCGTATCCCGCGCGCACATCATCCGTACC |
| | | GCGCGCGAATTTGCCGATAACGCCGATAAGACCCATGGCCGCTCG |
| | | ATGATTATCGTCGGTGCGGGCCTGAACCACTGGTTCCATCTGGAC |
| | | ATGAACTACCGCGGGCTGATCAACATGCTGATCTTCTGCGGCTGC |
| | | GTTGGCCAGAGCGGCGGCGGTTGGGCGCACTACGTTGGTCAGGA |
| | | AAAACTGCGTCCGCAGACCGGCTGGCAGCCGCTGGCGTTCGGCCT |
| | | TGACTGGCAGCGTCCGGCACGCCACATGAACAGTACATCGTACTT |
| | | CTATAACCACTCCAGCCAGTGGCGCTATGAAACCGTCACCGCGCA |
| | | GGAACTGCTGTCGCCGATGGCGGATAAATCCCGCTACAGCGGAC |
| | | ATCTGATCGACTTCAACGTGCGCGCTGAGCGTATGGGCTGGCTGC |
| | | CGTCGGCGCCGCAGCTGGGCGTGAACCCGCTGCGTATTGCTGACG |
| | | AAGCGAAGAAAGCCGGCATGACGCCGGTCGATTACACCGTGAAA |
| | | TCGCTGAAAGAGGGGTCTATTCGCTTTGCGGCCGAGCAGCCGGAG |
| | | AACGGTAAAAACCATCCCCGTAACCTGTTTATCTGGCGTTCCAAC |
| | | CTGCTGGGCTCCTCCGGTAAAGGCCATGAATATATGCTCAAGTAC |
| | | CTGCTGGGTACCGAGAACGGTATTCAGGGCAAAGACCTTGGCAA |
| | | GCAGGGCGGCGTGAAGCCGGAAGAAGTCGAATGGCGGGATAACG |
| | | GTCTCGACGGCAAACTGGATCTGGTCGTCACGCTCGACTTCCGTC |
| | | TGTCGAGCACCTGTCTGTACTCCGACATCGTACTGCCGACCGCAA |
| | | CCTGGTACGAAAAAGACGATATGAATACCTCGGATATGCATCCGT |
| | | TTATTCACCCGCTGTCTGCCGCCGTTGACCCGGCCTGGGAATCCA |
| | | AAAGCGACTGGGACATCTATAAAGGTATCGCTAAGAAATTCTCTG |
| | | AAGTCTGCGTAGGCCACCTCGGCAAAGAAACCGACGTCGTGACG |
| | | CTGCCGATTCAGCACGATTCCGCCGCGGAAATGGCGCAGCCGTTT |
| | | GACGTTAAAGACTGGAAAAAAGGCGAATGTGACCTGATCCCAGG |
| | | TAAAACCGCGCCGCATATTATTCCGGTCGAGCGTGATTATCCGGC |
| | | GACCTACGAGCGTTTCACCTCTATCGGCCCGCTGCTGGAAACCAT |
| | | CGGCAACGGCGGTAAAGGTATCGCCTGGAATACCCAGAGCGAGA |
| | | TGGACCTGCTGCGTAAGCTCAACTACACCAAGGCGGAAGGCCCG |
| | | GCGAAAGGCCAGCCGAAGCTGGAGACCGCTATTGACGCCGCTGA |
| | | GATGATCCTCACCCTGGCGCCGGAAACTAACGGTCAGGTAGCCGT |
| | | GAAGGCGTGGAAAGCGCTGAGCGAATTTACCGGCCGCGACCATG |
| | | CGCATCTGGCGCTGAACAAAGAAGACGAGAAGATCCGTTTTCGC |
| | | GATATCCAGGCGCAGCCGCGTAAGATCATCTCCAGCCCGACCTGG |
| | | TCTGGCCTGGAAGACGAACATGTCTCCTATAACGCCGGTTATACC |
| | | AACGTTCACGAGCTGATCCCATGGCGTACGCTGTCCGGCCGTCAG |
| | | TCGCTGTATCAGGATCACCAGTGGATGCGCGACTTTGGCGAAAGC |
| | | CTGCTGGTCTATCGTCCGCCGATTGACACCCGTTCGGTGAAAGCG |
| | | GTGATGGGCGAGAAGTCCAACGGCAATAAGGAGAAGGCGCTGAA |
| | | CTTCCTGACGCCGCACCAGAAGTGGGGGATTCACTCTACTTATAG |
| | | CGATAACCTGCTGATGCTGACTCTGTCGCGCGGCGGGCCGATCGT |
| | | CTGGATGAGCGAAGCGGATGCGAAAGATCTGGGTATCGAGGATA |
| | | ACGACTGGATCGAAGTCTTCAACGCCAACGGCGCCCTGACGGCG |
| | | CGTGCGGTAGTGAGCCAGCGCGTACCGGCAGGAATGACCATGAT |
| | | GTACCACGCGCAGGAACGTATCGTTAACCTGCCGGGTTCTGAAGT |
| | | TACCGGTCAGCGCGGGGGGATCCACAACTCGGTTACCCGTATTAC |
| | | GCCAAAACCGACCCATATGATCGGCGGCTACGGCCACCTGGCGT |
| | | ACGGCTTTAACTACTATGGCACCGTCGGTTCCAACCGCGATGAGT |
| | | TTGTTGTGGTACGTAAGATGAAGAACATTAACTGGTTAGACGGCG |
| | | AAGGTAATGACCAGGTACAGGAGAGCGTAAAATGA |
| nitrate | narY, narH, | >ADJCKNHF_03586 Respiratory nitrate reductase 1 beta chain |
| reductase/ | nxrB | ATGAAAATTCGTTCACAAGTCGGCATGGTGCTGAATCTCGATAAA |
| nitrite | | TGCATCGGCTGTCACACCTGCTCCGTAACCTGCAAAAACGTCTGG |
| oxidoreductase, | | ACCAGTCGTGAAGGGATGGAGTACGCCTGGTTTAACAACGTAGA |
| beta | | AAGTAAGCCGGGCGTCGGTTTCCCGAACGACTGGGAAAACCAGG |
| subunit | | AAAAATGGAAAGGCGGCTGGATCCGTAAAATCAACGGTAAACTG |
| [EC:1.7.5.1 | | CAGCCGCGCATGGGCAACCGCGCGATGCTGCTGGGTAAAATCTTC |
| 1.7.99.-] | | GCCAACCCGCATCTGCCGGGAATCGATGATTACTACGAGCCGTTT |
| | | GATTTTGACTACCAGAATCTGCATAACGCGCCGGAAAGCAAACAT |
| | | CAGCCGATCGCTCGTCCGCGTTCGCTGATTACCGGCCAGCGGATG |
| | | GACAAAATTACCAGCGGCCCGAACTGGGAAGAGATCCTCGGCGG |
| | | CGAGTTTGAAAAACGCGCCAAAGACCAGAACTTCGAAAACATGC |
| | | AGAAGGCGATGTACGGCCAGTTCGAAAACACCTTCATGATGTATC |
| | | TGCCGCGCTTATGCGAGCACTGCCTGAACCCGGCGTGCGTCGCGA |
| | | CCTGCCCGAGCGGTGCCATCTATAAGCGTGAAGAAGACGGTATTG |
| | | TCCTGATCGACCAGGATAAGTGCCGCGGCTGGCGTATGTGCATCA |
| | | CCGGCTGCCCGTACAAGAAGATCTATTTCAACTGGAAGAGCGGG |
| | | AAATCCGAGAAATGCATCTTCTGCTACCCGCGTATTGAATCCGGG |
| | | ATGCCGACGGTGTGCTCCGAAACCTGTGTGGGACGTATTCGCTAT |
| | | CTCGGTGTCCTGCTCTACGACGCGGACGCGATTGAAAACGCAGCC |
| | | AGCACCGAGAACGAGAAAGATCTGTATCAGCGTCAACTGGACGT |
| | | CTTCCTCGATCCTAACGATCCAAAAGTGATTGAACAGGCATTAAA |
| | | AGATGGTGTACCGCAGGGCGTAATTGAAGCCGCGCAGCAGTCGC |
| | | CGGTTTACAAAATGGCGATGGACTGGAAGCTGGCGCTGCCGCTGC |
| | | ACCCGGAATATCGCACCTTGCCAATGGTCTGGTACGTGCCGCCGC |
| | | TGTCGCCGATTCAGTCCGCCGCCGATGCGGGCGAACTGGGTAGCA |
| | | ACGGGATCCTGCCGGATGTAGAAAGCCTGCGTATTCCAGTCCAGT |
| | | ATCTGGCGAACCTGCTGACCGCGGGCGATACCCAGCCTGTACTGC |
| | | TGGCGCTGAAACGTATGCTGGCGATGCGCCACTTCAAACGTGCGG |
| | | AAACCGTCGACGGCATCGTCGATACCCGCGCGCTGGAAGAGGTC |
| | | GGGCTGAGCGAAGCGCAGGCGCAGGAGATGTACCGTTATCTGGC |
| | | TATCGCCAACTACGAAGATCGTTTCGTGGTGCCGAGCAGCCATCG |
| | | CGAACAGGCTCGCGATGCCTTCCCGGAGAAGAGCGGTTGCGGCTT |
| | | TACCTTCGGCGATGGTTGCCACGGCTCTGACAGCGAATTCAACCT |
| | | GTTTAACAGCCGTCGCATTGACGCCATTGACGTTACCAGCAAAAC |
| | | GGAGCCGCACGCATGA |
| nitrate | narJ/narW | >ADJCKNHF_03587 Nitrate reductase molybdenum cofactor |
| reductase | | assembly chaperone NarJ |
| molybdenum | | ATGATTGAACTCGTGATTGTGTCGCGTCTGCTCGAATACCCTGAC |
| cofactor | | GCTGCCTTATGGCAGCATCAGCAGGAGATGTTCGAGGCTCTCGCG |
| assembly | | TCATCGGAAAAACTCGCCAAAGAAGATGCCCAGGCGCTGGGCGT |
| chaperone | | TTTCCTGCGCGATTTAGTCGCTCAGGATCCGCTGGACGCCCAATC |
| NarJ/NarW | | GGCGTATAGCGAGCTGTTTGACCGCGGCCGCGCCACCTCGCTGCT |
| | | GCTGTTTGAACATGTTCACGGCGAGTCCCGCGATCGCGGGCAGGC |
| | | GATGGTCGACCTGATGGCGCAGTACGAGCGCCACGGTCTGCTGCT |
| | | GGATAGCCATGAGCTGCCGGATCACCTGCCGCTGTACCTTGAGTA |
| | | TCTGGCGCAGTTGCCGGAAGAGGAAGCGCTTGGCGGCCTGCGGG |
| | | ACGTTGCGCCAATCCTTGGCCTGCTCAGCGCGCGTCTGCAACAGC |
| | | GCGAGAGCCGCTATGCGGTGTTGTTCGAGCTGCTGTTGAAGCTTG |
| | | CCAATACTCAGGTCGATAGCCAGAAAGTGGCGGAGAAGATTGCC |
| | | GACGAGGCCCGCGACGATACACCGCAGGCGCTGGATGCCGTCTG |
| | | GGAAGAAGAGCAGGTCAAATTCTTTGCCGACCAGGGCTGCGGCG |
| | | AGTCGGAAATCTCCGCTCACCAGCGCCGTTTTGCCGGAGCCGTGG |
| | | CCCCGCAATATTTGAATATCTCTAACGGAGGACAGCACTAA |
| nitrate | narI, narV | >ADJCKNHF_03588 Respiratory nitrate reductase 1 gamma chain |
| reductase | | ATGCACTTCCTGAATATGTTCTTCTTTGACATCTATCCGTACATTG |
| gamma | | CGGGTTCAGTGTTTCTTATTGGCAGCTGGCTGCGCTACGACTACG |
| subunit | | GCCAGTACACCTGGCGCGCTGCCTCCAGTCAGATGCTGGATCGTA |
| [EC:1.7.5.1 | | AAGGGATGAACCTGGCGTCAAACCTGTTCCATATCGGGATCCTGG |
| 1.7.99.-] | | GTATTTTTGCCGGTCACTTCCTCGGTATGCTGACCCCGCACTGGAT |
| | | GTATGAGTCCTTCCTGCCGATCGACGTGAAGCAGAAAATGGCGAT |
| | | GTTTGCCGGCGGGGCGTGCGGCGTGATGACGTTGGTCGGCGGTTT |
| | | ACTGCTGCTCAAGCGCCGTCTGCTGAGCCCGCGCGTTCGTGCTAC |
| | | CACCACCACAGCGGATATCCTGATCCTCTCGTTGCTGATGGTGCA |
| | | ATGCGCGCTGGGTCTGCTGACCATTCCATTCTCCGCCCAGCATAT |
| | | GGACGGTAGCGAAATGATGAAACTGGTCGGCTGGGCGCAGTCGG |
| | | TGGTGACCTTCCACGGCGGAGCTTCGCAGCATCTCGATGGCGTAG |
| | | CTTTTGTCTTCCGAGTTCACCTGGTGCTGGGGATGACGCTGTTCCT |
| | | GCTGTTCCCGTTCTCGCGCCTGGTTCACATCTGGAGCGCGCCGGT |
| | | CGAGTACCTGACGCGCAAATACCAGATTGTGCGCGCGCGTCGCTA |
| | | G |
| acetolactate | alsS | >ADJCKNHF_03736 Acetolactate synthase, catabolic |
| synthase, | | ATGGACAAACAGTATCCGCAGCGCCAGTGGGCGCACGGCGCCGA |
| catabolic | | TCTGGTCGTCAGCCAACTGGAAGCGCAAGGCGTACGGCAGGTCTT |
| | | CGGGATCCCCGGCGCTAAAATCGATAAGGTTTTCGACTCGTTGCT |
| | | GGACTCCTCAATCCGCATTATTCCGGTACGTCACGAGGCCAACGC |
| | | CGCCTTTATGGCCGCCGCGGTCGGGCGCATTACCGGCAAAGCGGG |
| | | CGTCGCGCTGGTGACCTCCGGACCCGGTTGTTCCAACCTGATAAC |
| | | CGGGATGGCCACCGCCAATAGCGAAGGCGACCCGGTGGTGGCGC |
| | | TGGGCGGCGCGGTCAAACGCGCGGATAAAGCCAAACAGGTACAC |
| | | CAGAGTATGGACACGGTGGCGATGTTCAGCCCGGTCACCAAATA |
| | | CGCGGTAGAAGTGACCTCGCCGGATGCGCTGGCGGAAGTGGTTTC |
| | | TAACGCTTTTCGCGCCGCCGAGCAGGGTCGCCCGGGCAGCGCCTT |
| | | CGTCAGTCTGCCGCAGGATGTGGTCGATGGTCCGGTGACCGGCAA |
| | | AGTCCTACCCGCCAGCAGCGCGCCGCAGATGGGCGCCGCGCCTG |
| | | ACGAGGCAATCAATCAGGTTGCGAAGTTGATTGCCCAGGCGAAG |
| | | AATCCGGTGTTCCTGCTTGGATTAATGGCCAGCCAGACGGAAAAC |
| | | AGCGCCGCGCTGCATCGTTTGCTGGAAACCAGCCATATTCCGGTC |
| | | ACCAGCACCTATCAGGCCGCCGGGGCGGTCAATCAGGATAACTTC |
| | | TCGCGCTTCGCCGGGCGCGTCGGGCTGTTTAACAATCAGGCCGGT |
| | | GACCGCTTATTGCAACTGGCCGACCTGGTTATCTGCATCGGCTAT |
| | | AGCCCGGTGGAATACGAACCGGCGATGTGGAACAGCGGCAACGC |
| | | GACGCTGGTCCACATCGACGTACTGCCCGCCTATGAAGAGCGTAA |
| | | CTACACGCCGGATGTCGAGCTGGTGGGCGACATCGCCGGCACGCT |
| | | GAACAAGCTGGCGCAAAATATCGATCATCGGCTGGTGCTCTCGCC |
| | | GCAGGCCGCTGAAATCCTCCACGACCGCCAGCATCAACGCGAAC |
| | | TGCTTGACCGCCGCGGAGCGCAGTTGAATCAGTTTGCCCTGCACC |
| | | CGCTGCGCATCGTTCGCGCCATGCAGGATATCGTCAACAGCGACG |
| | | TCACGCTGACGGTCGATATGGGGAGCTTCCATATCTGGATCGCCC |
| | | GCTATCTCTACAGCTTCCGCGCCCGTCAGGTGATGATCTCCAACG |
| | | GTCAGCAGACCATGGGCGTCGCCCTGCCGTGGGCCATCGGGGCCT |
| | | GGCTGGTCAATCCGCAGCGCAAAGTGGTCTCGGTCTCCGGCGATG |
| | | GCGGTTTTCTGCAATCCAGCATGGAGCTGGAAACGGCGGTCCGCC |
| | | TGAAAGCCAACATCCTGCATCTTATCTGGGTCGATAACGGCTACA |
| | | ACATGGTCGCTATCCAGGAAGAGAAAAAATATCAACGCCTGTCC |
| | | GGCGTCGAGTTCGGTCCTATGGATTTTAAAGCCTATGCCGAATCC |
| | | TTCGGCGCGAAAGGGTTTGCGGTGGAAAGCGCTGAGGCGCTGGA |
| | | GCCGACGCTACGCGCGGCGATGGACGTCGACGGCCCGGCGGTGG |
| | | TCGCCATCCCCGTGGATTACCGTGATAACCCGCTGCTGATGGGCC |
| | | AGCTACACCTGAGTCAAATTCTTTAA |
| acetolactate | alsD, budA, | >ADJCKNHF_03737 Alpha-acetolactate decarboxylase |
| decarboxylase | aldC | ATGAATCATGCTTCAGATTGCACCTGCGAAGAGAGTCTGTGTGAA |
| [EC:4.1.1.5] | | ACGCTACGCGCGTTTTCCGCTCAGCATCCCGATAGCGTGCTGTAT |
| | | CAAACTTCGCTGATGAGCGCCCTGCTCAGCGGCGTCTACGAAGGT |
| | | ACCACCACCATTGCGGACCTGCTGAAGCACGGTGATTTCGGGCTC |
| | | GGCACTTTTAATGAACTCGACGGCGAGCTGATCGCGTTTAGCAGC |
| | | CAGGTTTATCAACTGCGTGCCGACGGCAGCGCGCGTAAAGCGCGT |
| | | CCGGAACAGAAAACGCCGTTTGCGGTGATGACCTGGTTTCAGCCG |
| | | CAGTACCGTAAAACCTTTGACCATCCGGTCAGCCGCCAGCAGCTG |
| | | CATGAGGTTATTGACCAGCAAATTCCTTCCGACAATCTGTTCTGC |
| | | GCGCTGCGAATCGATGGTCATTTCCGCCACGCCCATACCCGCACC |
| | | GTGCCTCGTCAGACGCCGCCCTACCGGGCGATGACCGACGTGCTC |
| | | GACGATCAGCCGGTTTTCCGCTTTAACCAGCGTGACGGCGTACTG |
| | | GTCGGTTTTCGGACCCCCCAGCATATGCAGGGAATTAACGTCGCC |
| | | GGCTATCACGAACACTTCATTACCGATGACCGCCAGGGCGGCGGC |
| | | CACCTGCTGGACTACCAGCTCGACCATGGGGTATTGACCTTCGGC |
| | | GAAATTCATAAGCTGATGATCGACCTTCCCGCCGACAGCGCGTTC |
| | | CTGCAGGCCAATTTGCATCCCGATAATCTCGATGCCGCCATCCGT |
| | | TCAGTAGAAAGTTAG |
| quinoprotein | gcd | >ADJCKNHF_03828 Quinoprotein glucose dehydrogenase |
| glucose | | ATGGCAGAAACAAAATCTCAACAATCACGGTTACTTGTCACGCTG |
| dehydrogenase | | ACGGCGCTGTTTGCCGCCTTCTGTGGCCTGTATCTGTTAATCGGTG |
| [EC:1.1.5.2] | | GGGTATGGCTGGCCGCTATTGGCGGTTCCTGGTACTACCCTATCG |
| | | CAGGCCTGGTGATGCTGGCCGTCACCGTTATGCTGTTCCGCGGCA |
| | | AGCGCGCTGCGCTGTGGCTGTACGCCGCCCTGCTGCTGGCAACCA |
| | | TGATTTGGGGGGTATGGGAAGTCGGTTTCGACTTCTGGGCGCTGA |
| | | CGCCGCGTAGCGACATCCTGGTCTTCTTCGGCATCTGGCTGATTCT |
| | | GCCATTTGTCTGGCGTCGTCTGCCGGTCCCTTCCGCCGGTGCCGTT |
| | | GGCGGTCTGGTTATCGCCCTGCTGATTAGCGGCGGGATCCTGACC |
| | | TGGGCCGGTTTCAACGATCCGCAGGAAGTGAACGGTACGCTGAG |
| | | CGCTGACGCGACGCCGGCTGCGCCGATTTCTACCGTCGCCGATAG |
| | | CGACTGGCCGGCTTATGGCCGCAACCAGGAAGGCCAGCGTTATTC |
| | | ACCGCTGAAGCAAATTAATACCGATAACGTGAAGAACCTGAAGG |
| | | AAGCCTGGGTATTCCGCACCGGCGACCTGAAGCAGCCGAACGAT |
| | | CCGGGTGAAATCACTAACGAAGTGACGCCGATTAAAGTTGGCGA |
| | | CATGCTGTATCTGTGTACCGCGCACCAGCGTCTGTTCGCGCTTGA |
| | | CGCGGCCACCGGTAAAGAGAAGTGGCATTTTGACCCGCAGCTGA |
| | | ACGCCGATCCGTCGTTCCAGCACGTGACCTGCCGTGGCGTCTCCT |
| | | ATCATGAAGCCAAAGCAGATAACGCGCCTGCCGACGTCGTCGCC |
| | | GACTGCCCGCGCCGTATTATTCTGCCGGTCAACGATGGCCGTCTG |
| | | TTCGCGGTGAACGCCGACAACGGTAAGCTGTGCGAAACCTTTGCC |
| | | AACAAAGGCATTCTCAACCTGCAAACCAATATGCCGGTAACCAC |
| | | GCCGGGTATGTATGAACCGACGTCGCCGCCGATTATCACCGATAA |
| | | GACTATCGTCATTGCCGGCGCGGTAACCGATAACTTCTCAACCCG |
| | | CGAGCCATCAGGCGTAATCCGCGGCTTTGATGTGAATACCGGTAA |
| | | ACTGCTGTGGGCCTTCGACCCGGGTGCGAAAGACCCTAATGCGAT |
| | | CCCGAGCGATGAGCATCACTTCACACTCAATTCACCTAACTCCTG |
| | | GGCGCCTGCCGCCTATGACGCGAAGCTGGATTTAGTCTATCTGCC |
| | | GATGGGCGTTACCACGCCGGATATCTGGGGCGGCAATCGCACGC |
| | | CGGAACAGGAACGTTACGCCAGCTCTATCGTAGCGCTGAACGCA |
| | | ACCACCGGGAAACTGGCATGGAGCTACCAAACCGTACACCACGA |
| | | TCTGTGGGATATGGATATGCCGTCGCAGCCGACGCTGGCCGACAT |
| | | TGATGTGAACGGTAAAACCGTGCCGGTGATTTACGCTCCGGCCAA |
| | | AACCGGCAACATCTTCGTGCTGGATCGCCGTAATGGCGAACTGGT |
| | | GGTCCCGGCGCCGGAAAAACCGGTTCCGCAAGGTGCGGCGAAAG |
| | | GCGATTATGTTGCTAAAACCCAGCCGTTCTCCGAGCTGAGCTTCC |
| | | GTCCGAAGAAAGACCTGACCGGCGCAGATATGTGGGGCGCCACC |
| | | ATGTTTGACCAACTGGTGTGCCGCGTTATCTTCCATCAGATGCGCT |
| | | ATGAAGGTATCTTCACTCCACCGTCTGAACAGGGCACGCTGGTCT |
| | | TCCCGGGGAACCTGGGGATGTTTGAATGGGGCGGTATCTCCGTCG |
| | | ATCCAAACCGTCAGGTGGCTATCGCCAACCCGATGGCGCTGCCGT |
| | | TCGTCTCTAAATTGATCCCACGCGGCCCGGGCAACCCGATGGAAC |
| | | CGCCAAAAGACGCGAAAGGCTCTGGTACCGAGTCCGGCGTTCAG |
| | | CCGCAGTATGGCGTCCCGTTCGGCGTCACGCTGAACCCGTTCCTG |
| | | TCGCCGTTTGGCCTGCCGTGTAAACAACCGGCATGGGGTTATATC |
| | | TCGGCGCTGGATCTGAAGACCAACGAAGTAGTGTGGAAAAAACG |
| | | CATCGGTACGCCGCAGGATAGTCTGCCGTTCCCGCTGCCTGTCCC |
| | | GCTGCCATTCAATATGGGTATGCCGATGCTGGGCGGACCGATCTC |
| | | AACCGCCGGTAACGTGCTGTTTATCGCCGCCACCGCCGATAACTA |
| | | CCTGCGCGCTTACAACATGAGTAATGGTGAAAAACTGTGGCAGG |
| | | CGCGTCTGCCTGCAGGTGGCCAGGCGACGCCGATGACCTATGAA |
| | | GTGAATGGCAAGCAGTACGTTGTCATCTCTGCCGGCGGTCATGGC |
| | | TCGTTCGGTACTAAAATGGGCGACTACATCGTTGCCTATGCGTTA |
| | | CCGGATGACGCGAAATAA |
| spermidine | speE, SRM | >ADJCKNHF_03833 Polyamine aminopropyltransferase |
| synthase | | ATGGCCGAGAGTAATCAGTGGCATGAGACGCTGCACGACCATTTT |
| [EC:2.5.1.16] | | GGTCAATATTTTACCGTTGATAACGTACTGTACCATGAGAAGACC |
| | | GATCATCAGGATTTAATCATCTTCGAAAATGCGGCATTTGGCCGC |
| | | GTGATGGCGCTTGATGGCGTGGTGCAAACCACCGAGCGCGATGA |
| | | GTTTATTTACCATGAAATGATGACCCACGTTCCGCTGCTGGCCCA |
| | | TGGTCACGCAAAGCACGTGCTGATCATCGGCGGCGGCGATGGCG |
| | | CGATGCTGCGTGAAGTCTCCCGCCACCAACATATCGAAACCATTA |
| | | CCATGGTGGAAATCGACGCCGGAGTGGTATCGTTCTGCCGTCAGT |
| | | ACCTTCCAAACCATAACGCCGGCGCCTATGACGACCCGCGCTTTA |
| | | CGCTGGTGATTGATGACGGCGTGAACTTCGTCAACCAAACGTCAC |
| | | AAACTTTTGATGTCATTATCTCCGACTGTACTGACCCGATCGGCCC |
| | | CGGTGAGAGCCTGTTTACCTCGGCGTTTTATGCTGGCTGTAAACG |
| | | TTGCCTGAACCCCGGCGGAATTTTCGTTGCCCAGAACGGCGTAAG |
| | | CTTCCTGCAGCAGGATGAAGCGGTAGGCAGCCATCGCAAACTCA |
| | | GCCACTATTTCACCGATGTCAGCTTCTACCAGGCGGCGATCCCGA |
| | | CCTACTATGGCGGTATCATGACCTTTGCCTGGGCGACCGATAATG |
| | | AAGCCCTGCGTCATTTGTCGACGGAAATTATTCAGGCCCGCTTCC |
| | | ATAGCGCCGGGCTGAAATGCCGTTATTACAATCCGGCAATTCATA |
| | | CCGCGGCTTTCGCGCTACCGCAATACCTGCTGGATGCGCTGACCG |
| | | CCAGCTAA |
| S- | speH, | >ADJCKNHF_03834 S-adenosylmethionine decarboxylase proenzyme |
| adenosylmeth | speD, | TTGAAAAAGCTTAAGCTGCACGGCTTCAACAACCTGACTAAAAGC |
| ionine | AMD1 | CTGAGTTTTTGTATTTACGATATCTGCTACGCTAAAACCGCCGAA |
| decarboxylase | | GAGCGCGATGGCTATATAGCCTATATCGATGAACTCTATAATGCC |
| [EC:4.1.1.50] | | AATCGCCTGACGGAAATCCTCTCCGAGACCTGTTCAATCATTGGC |
| | | GCCAACATACTCAACATCGCACGCCAGGATTATGAACCGCAGGG |
| | | CGCAAGCGTCACCATTTTGGTTAGCGAAGAGCCTATCGATCCGCA |
| | | GCTTATCGATCAGAGCGAGCATCCGGGCCCGCTGCCTGAGACCGT |
| | | GGTCGCACATCTCGATAAAAGCCACATCTGCGTGCATACCTATCC |
| | | GGAAAGCCATCCGGAAGGCGGTCTGTGCACCTTCCGCGCCGATAT |
| | | TGAAGTCTCCACCTGTGGAGTGATTTCACCGCTGAAGGCACTGAA |
| | | CTACCTGATTCACCAACTGGAATCCGATATCGTAACCATTGATTA |
| | | TCGCGTGCGTGGTTTCACCCGTGATATCAACGGTATGAAGCATTT |
| | | TATCGATCATGAAATCAACTCTATTCAGAACTTCATGTCAGAAGA |
| | | TATGAAGTCGCTCTATGACATGGTGGACGTGAACGTCTATCAGGA |
| | | AAATATGTTCCATACCAAAATGATGCTGAAAGAGTTCGATCTGAA |
| | | ACACTACATGTTCCACACCAAACCGGAAGAGTTAAGCGAACAAG |
| | | AGCGCAAGGTGATTACCGACCTGCTGTGGAAAGAGATGCGGGAA |
| | | ATTTATTACGCCCGCAATATCCCTGCGGTGTAA |
| acetolactate | ilvH, ilvN | >ADJCKNHF_03878 Acetolactate synthase isozyme 3 small subunit |
| synthase I/III | | ATGCGCCGGATATTATCTGTATTACTGGAGAACGAATCGGGAGCA |
| small subunit | | TTATCCCGGGTGATCGGCCTCTTTTCGCAGCGCGGCTACAATATT |
| [EC:2.2.1.6] | | GAAAGCCTGACGGTAGCGCCAACCGACGATCCGACGTTGTCCCG |
| | | CATGACCATCCAGACGGTGGGCGACGAAAAAGCCATTGAGCAGA |
| | | TCGAAAAGCAATTACATAAGCTGGTGGACGTGCTGCGGGTCAGC |
| | | GAACTGGGTCAGGGCTCCCACGTCGAACGTGAAATCATGCTGGTG |
| | | AAAGTGCAGGCCAGCGGTTATGGCCGCGAAGAGGTGAAACGCAA |
| | | CACCGAGATCTTCCGCGGGCAGATCATTGACGTCACGCCATCTAT |
| | | TTATACCGTGCAGTTGGCCGGAACCAGCGATAAGCTGGATGCGTT |
| | | CCTGGCTTCTTTACGCGATGTCGCGCGCATTGTTGAAGTGGCGCG |
| | | ATCCGGAGTAGTCGGCCTGTCGCGCGGCGATAAAATCATGCGCTA |
| | | A |
| acetolactate | ilvB, ilvG, | >ADJCKNHF_03879 Acetolactate synthase isozyme 3 large subunit |
| synthase | ilvI | ATGGAGATGTTGTCAGGAGCCGAGATGGTCGTCCAATCGCTTGTC |
| I/II/III large | | GATCAGGGCGTCAAGCAAGTATTCGGCTATCCCGGAGGCGCGGT |
| subunit | | CCTCGATATCTATGATGCATTACATACCCTCGGCGGCATTGACCA |
| [EC:2.2.1.6] | | TGTGCTGGTCCGCCATGAGCAGGCGGCGGTGCATATGGCCGACG |
| | | GACTGGCGCGCGCAACCGGCGAAGTCGGGGTGGTGCTGGTGACC |
| | | TCCGGCCCGGGAGCGACTAACGCTATTACCGGCATTGCAACGGCT |
| | | TACATGGATTCTATTCCGCTGGTCATTCTTTCCGGGCAGGTCGCCA |
| | | CCTCGCTGATTGGCTACGATGCCTTCCAGGAGTGCGATATGGTCG |
| | | GTATCTCGCGCCCGGTGGTTAAACACAGCTTCCTCGTGAAACAGA |
| | | CTGAAGATATCCCCGGGATTTTAAAGAAAGCCTTCTGGCTGGCGG |
| | | CCAGCGGCCGACCGGGGCCGGTAGTGGTCGATTTGCCGAAGGAT |
| | | ATTCTCAATCCGGCGAAAAAGCTGCCTTATGTTTGGCCGGATGCG |
| | | GTCAGCATGCGTTCCTATAACCCGACAACCAGCGGTCATAAAGGG |
| | | CAGATCAAACGTGCGTTGCAAACGCTGGTGGCGGCGAAAAAACC |
| | | GGTCGTGTACGTTGGCGGCGGGGCAATCAATTCGCAGTGCGAAG |
| | | CACAGCTGCGTACGCTGGTCGAAAAGCTGAAGCTACCGGTGGTCT |
| | | CTTCATTAATGGGTTTAGGCGCTTTTCCGGCGACTCATCAGCAGG |
| | | CGCTGGGGATGTTGGGGATGCACGGTACCTATGAAGCCAACATG |
| | | ACCATGCATCATTCCGACGTTATTTTTGCAGTCGGCGTCCGCTTTG |
| | | ACGATCGCACCACCAACAATTTGGCTAAGTATTGCCCGAACGCCA |
| | | CGGTGCTGCATATTGATATCGATCCCACGTCGATCTCCAAAACCG |
| | | TCCCGGCAGATGTGCCGATCGTCGGCGACGCGCGCCAGGTGCTTG |
| | | ACCAGATGTTTGATCTGCTTGAGCAGGAAGAGAGCCAGCAACCG |
| | | CTGGATGAGATCCGCGACTGGTGGCAGCAGATCGAACAGTGGCG |
| | | TTCCCGTCACTGTCTGCAATACGACACGCAAAGCGGCAAGATCAA |
| | | ACCGCAGGCGGTGATTGAGGCTATCTGGCGCCTGACCAATGGTGA |
| | | TGCTTACGTGACTTCCGACGTTGGACAGCATCAGATGTTCGCCGC |
| | | GCTCTATTATCCATTCGATAAACCACGGCGTTGGATTAACTCCGG |
| | | CGGCCTCGGCACGATGGGCTTTGGCCTGCCAGCGGCACTGGGCGT |
| | | GAAGATGGCGCTACCGCAAGAAACCGTGATCTGCGTGACCGGCG |
| | | ATGGCAGCATCCAGATGAACATTCAGGAGCTTTCCACCGCCCTGC |
| | | AGTATGAACTGCCGGTGCTGGTGCTGAACCTGAATAACCGCTACC |
| | | TCGGAATGGTAAAGCAGTGGCAGGATATGCTCTATTCTGGCCGCC |
| | | ATTCGCAGTCCTATATGGAATCGCTGCCGGACTTCGTTCGCGTCG |
| | | CCGAGGCTTATGGCCACGTTGGTATTCGTATCAGCGAGCCGCAAG |
| | | AGCTGGAAGCGAAGCTGGCGGAAGCCCTGGAGCAGGTGCGTAAT |
| | | AATCGTCTGGTGTTCGTTGATGTTACGGTTGATGGCAGCGAGCAT |
| | | GTTTATCCCATGCAGATCCGCGGCGGCGGCATGGACGAAATGTGG |
| | | TTGAGCAAAACGGAGAGAACCTGA |
| inorganic | ppa | >ADJCKNHF_04028 Inorganic pyrophosphatase |
| pyrophosphatase | | ATGAGCTTACTCAACGTACCTGCGGGCAAAGAGCTGCCGGAAGA |
| [EC:3.6.1.1] | | CATCTACGTCGTTATCGAAATCCCAGCTAACGCAGACCCAATCAA |
| | | ATACGAAGTTGACAAAGAGAGCGGCGCACTGTTCGTTGACCGTTT |
| | | CATGTCCACTGCGATGTTCTATCCGTGCAACTACGGCTACATCAA |
| | | CCACACCCTGTCCCTGGACGGCGACCCGGTTGACGTGCTGGTCCC |
| | | GACTCCGTACCCGCTGCAACCAGGCTCAGTTATCCGCTGCCGTCC |
| | | GGTTGGCGTACTGAAAATGACCGACGAATCCGGTGAAGATGCCA |
| | | AGCTGGTTGCGGTACCGCACACCAAGCTGAGCAAAGAATACGAT |
| | | CACATCAAAGATGTGAACGACCTGCCGGAACTGCTGAAAGCCCA |
| | | GATCACTCACTTCTTCGAGCACTACAAAGATCTGGAAAAAGGCAA |
| | | GTGGGTTAAAGTCGACGGTTGGGACAACGCTGAAGCGGCTAAAG |
| | | CTGAAATCGTTGCTTCCTTCGAGCGCGCTAAACAGAAGTAA |
| hydrogen | hcnB | >ADJCKNHF_04202 Hydrogen cyanide synthase subunit HcnB |
| cyanide | | ATGAACACGCTGCGCTGTGACATTTTAATCCTCGGCGCCGGCCCC |
| synthase | | GCAGGGGTGGCGGCCGCGCTCTCCGCCGCCGCCTGCGATAAACA |
| HcnB | | GGTGATTATTCTCGACGATAATCCCGCCCCTGGCGGGCAAATCTG |
| [EC:1.4.99.5] | | GCGTGCCGGTCCGCAGGCGACGCAGCCCGCCCTGGCCCGGCACT |
| | | ACCGCGACGCCATCGCCGCGTCTGCCGCCATCCGGTTAGTGAACG |
| | | GCGCGCGGCTGATTGCCCGCCCCTCCGCGTACAGTGTGCTGTTTG |
| | | AAACCGCCGACGGCGGTGGTGTGGTTTACTGGCAGAAACTGATCC |
| | | TCTGCTGCGGCGCGCGCGAGCTGTCGCTGCCCTTTCCCGGTTGGA |
| | | CCTTACCCGGCGTGACCGGCGCGGGCGGCCTGCAGGCGCAAATC |
| | | AAACAGGGCCTTGCGCTGAAAGGAGAAAAGGTGGTGATTGCCGG |
| | | CAGCGGTCCGCTACTGCTGGCGGTGGCGGATACGGTGAACAAGG |
| | | CCGGAGGCGAAGTGACGAATATCATTGAGCAAGCGCCGCTACCG |
| | | GCACTGCTGCGCTTCGCCGGTGGGCTGTGGCGCTGGCCGCAGAAG |
| | | CTGCGCCAGTTAGCGATGCTGGCTCCGAAAGGCTATCTTAGCGGC |
| | | ACGCAGGTGATCCGCGCTCACGGCACAACGCGGCTGGAAGCCGT |
| | | TACGTTGCGCCAGCGCGGCGACGAGCGAACTATCGCCTGCGATCG |
| | | GCTGGCTATCGGCTACGGGCTGATACCCAATATCGAGACGGCGCT |
| | | GCTGTTTGGCTGCGCCACGGCGCAGGAGGCGATACAGGTTAACC |
| | | GCTGGCAGCAGACCAGCATCGCGGATATCTACGCCGCCGGCGAG |
| | | TGCACCGGTTTCGGCGGTAGCGAACTGGCGCTGGCGGAGGGCGA |
| | | AATCGCCGGTTTTGCCGCCGCCGGGGCCAGCCATCAAGCGCAGGC |
| | | GTTATTTGCCCGCCGCGCCCGCTGGCAGCGCTTCGCCGACGCCAT |
| | | AAACCGCGCCTTCCGGCTGGCGGAATCTTTGAAAAACGCGGCGA |
| | | CGCCGGAGAGCCTGCTGTGCCGCTGCGAGGATGTGCGCTGCGGC |
| | | GATGTGGCGGCAGCCGGAGGCTGGCCGCAGGCCAAACTTACCCA |
| | | GCGCTGCGGAATGGGCGCCTGTCAGGGGCGCACCTGCGCCGCCA |
| | | GCGCCCGCTGGTTATATGGCTGGCCGCTGCCGCAGCCGAGAGAAC |
| | | CGCTGGCCCCTGCCCGCGCGGAAACGCTTATTGCCCTCGCCAGGT |
| | | TGAGCGCCGAGCCGTAA |
| hydrogen | hcnA | >ADJCKNHF_04203 hypothetical protein |
| cyanide | | ATGAATGCCACGCTCACTATCATCGTTGATGGCGAAGCGCTGACG |
| synthase | | GTGCCGGAAGGGATCAGCGTGGCGGCGGCGCTGGCGTTGACCGG |
| HcnA | | CGACCCCACCACCCGCCAGGCGGTTAACGGCGGCCTCCGCGCGCC |
| [EC:1.4.99.5] | | GTTTTGCGGCATGGGCGTCTGCCAGGAGTGCCGGGTCACCGTCGA |
| | | TGGCCTACGGGTGCTGGCCTGCCAGACCCTGTGCCGGTCCGATAT |
| | | GCAAATAGAAAGGAGCCGCGATGAACACGCTGCGCTGTGA |
| hydrogen | hcnC | >ADJCKNHF_04204 Glycine oxidase |
| cyanide | | GTGAAGCAGTGCGACGTCATCGTGATCGGCGCCGGGATCATCGG |
| synthase | | CGCCGCCTGCGCGTGGCAGCTGGCGAAGCGGGGGCAGAGCGTGA |
| HcnC | | CGCTTATCGATGACGGTCAGCCTGGCGCGACGGCGGCCGGTATGG |
| [EC:1.4.99.5] | | GCCATCTGGTTTGCATGGATGACGACCCCGCCGAGCTTGCATTAT |
| | | CGGCATGGTCGCTGGAACGCTGGCGCGCTATCACGCCACGGATGC |
| | | CCGACAATTGCGCCTGGCGCGGCTGCGGTACGCTGTGGCTGGCGG |
| | | AAAGCGAAGAAGAGATGGCCGGGGCTGGCGACAAACAGCGGCG |
| | | GATGGCCGGCCATCAGGTGCACAGCGAACTCCAGACCCCGCAGC |
| | | AGATCGCCGGGCGCGAGCCGCTGCTGCGCGACGGGCTGGCCGGC |
| | | GGCCTGTGGGTGCCAGGCGATGGCATCGTCTACGCGCCGAACGTC |
| | | GCCCGCTGGCTGATTACCGATGCCGGAAACCATCTTACCTGCCTG |
| | | CGCGATAGCGTACAGACGATTGACGAGCCGCAGGTGCTGCTCGC |
| | | CAGCGGCAAACGGCTACAGGCGCGGGCGATCGTGGTGGCCTGCG |
| | | GCCTTGAAGCTAACGCGCTGTTAGCGGAAAACTGGCTGCGACCG |
| | | AAAAAAGGCCAACTGGCGATTACCGATCGCTATGGGCCGCAGGT |
| | | GCATCACCAGCTGGTTGAGCTGGGTTATGGCGCCAGCGCCCACGG |
| | | CGGCGGCACCTCGGTGGCGTTTAACCTTCAGCCGCGGCCAACCGG |
| | | CCAGCTGCTAATTGGCTCTTCGCGTCAGTTTGATAACCGCGAGCG |
| | | CGAGTTGGATCTGCCCTTGCTGGCGCAGATGCTCGATCGCGCCCG |
| | | CCACTTCGTGCCGGCGCTGGCGACGTTGAATATCATCCGCTGCTG |
| | | GAGCGGCCTGCGCGCCGCCTCTCCGGATGGCAATCCGTTGATCGG |
| | | CCCGCACCCCACCCGCCGTGGTTTATGGCTAGCGCTGGGTCATGA |
| | | AGGGCTGGGCGTCACCACTGCCCCGGCCTCGGCTGAACTGCTGGC |
| | | GGCGCAGATCCTCGATGAACGCTGCCCATTGGCTCCCGACGCCTG |
| | | GCTCCCGGCTCGTTTATCTCAACAGGAGGCGATCGCATGA |
| acetolactate | ilvH, ilvN | >ADJCKNHF_04581 Acetolactate synthase isozyme 2 small |
| synthase I/III | | subunit |
| small subunit | | ATGATGCAACATCAGGTCGCTTTACAGGCTCGCTTCAACCCCGAA |
| [EC:2.2.1.6] | | ACCTTAGAACGCGTGCTGCGCGTGGTGCGCCATCGCGGTTTTCAA |
| | | ATTTGCTCAATGAATATGGAAACCGCGTCGGATGCGCAAAACATA |
| | | AATATCGAGCTGACCGTTGCCAGCCAGCGGCCCGTCGAATTACTG |
| | | TTTAGTCAGTTACGCAAACTGGTCGACGTCGCCTGCGTCGAGATC |
| | | CAGCAACCCACATCACAACAAATCCGCGCCTGA |
| acetolactate | ilvB, ilvG, | >ADJCKNHF_04582 Acetolactate synthase isozyme 2 large |
| synthase | ilvI | subunit |
| I/II/III large | | ATGAACGGGGCGCAGTGGGTGGTACATGCTTTGCGAACACAGGG |
| subunit | | GGTCGACACGGTATTTGGCTATCCGGGTGGCGCGATTATGCCGGT |
| [EC:2.2.1.6] | | TTACGATGCTTTGTATGACGGCGGCGTGGAACACCTGCTGTGTCG |
| | | GCACGAGCAAGGCGCCGCAATGGCCGCCATCGGTTATGCCCGCG |
| | | CGACCGGCAAAACTGGTGTTTGCATCGCCACTTCCGGTCCTGGCG |
| | | CCACCAACCTGATCACCGGTTTGGCTGACGCGTTACTTGATTCTGT |
| | | ACCTGTTGTCGCCATCACCGGTCAAGTGGCGGCGCCGTTTATCGG |
| | | CACCGATGCTTTTCAGGAAGTGGACGTTCTCGGTTTGTCGCTGGC |
| | | CTGCACCAAACACAGTTTCCTCGTGCAGTCGTTGGAAGAGCTGCC |
| | | GCGCGTCATTGCGGAAGCTTTCCAGGTGGCAAACTCAGGCCGTCC |
| | | TGGCCCGGTACTGGTTGATATTCCAAAAGATATCCAGTTGGCTAA |
| | | AGGCGAATTAGATCCGCATTTCTCCACCGTCCCTGATGATGTTGA |
| | | GTTCCCGCACACACAAGTCGAGCAGGCGTTAGCGATGCTTGCGCA |
| | | GTCCCACAAGCCAATGCTGTACGTGGGTGGCGGTGTTGGAATGGC |
| | | ACAGGCGGTACCGGCCGTGCGCGAATTTCTGGCGGTGACGCAGA |
| | | TGCCGGTAACCTGCACTCTGAAAGGGTTGGGCGCCGTCGCCGCGG |
| | | ATTATCCGTATTACCTTGGCATGCTGGGTATGCACGGAACCAAGG |
| | | CAGCAAACCTGGCGGTGCAGGAGTGCGATTTATTAATCGCCGTCG |
| | | GCGCCCGTTTTGATGACCGGGTTACCGGCAAGCTGAATACCTTTG |
| | | CCCCGCACGCCAAAGTAATCCATATGGATATTGACCCGGCCGAGC |
| | | TGAACAAACTGCGCCAGGCGCACGTCGCCTTAACCGGAGATTTAA |
| | | ACGCCATGCTGCCGGCGTTGCAGCAGCCGTTGGCCATCGATGCGT |
| | | GGCGCGAGCGCAACGCGCAGCTGCGCGCGGAGCACGCCTGGCGT |
| | | TACGATCATCCCGGCGAGGCAATCTACGCGCCGCTGTTGCTCAAG |
| | | CAGCTTTCCGATCGCAAACCGGCGGATTGCGTCGTGACGACCGAT |
| | | GTCGGCCAGCACCAAATGTGGTCGGCCCAGCACATGACCTATACC |
| | | CGCCCGGAAAACTTCATCACTTCCAGCGGCCTCGGCACCATGGGT |
| | | TTTGGTCTGCCGGCAGCCGTTGGCGCGCAGGTGGCTCGCCCGGAC |
| | | GATACGGTTATCTGTATCTCCGGCGATGGCTCTTTCATGATGAAC |
| | | GTGCAGGAGCTGGGCACCGTTAAGCGCAAGCAATTACCGTTGAA |
| | | AATCGTGCTGCTGGATAACCAACGTTTAGGCATGGTTCGCCAGTG |
| | | GCAGCAGCTGTTTTTCCAGGAGCGTTACAGCGAAACCACGCTTAC |
| | | CGATAATCCTGATTTTCTTACGCTGGCCAGCGCTTTTGGCATTCCA |
| | | GGCCAGCACATCACCCGTAAAGACCAGGTTGAAGCGGCACTCGA |
| | | CACCATGCTTTCGAGCCAGGGGCCATACCTGCTTCATGTCTCAAT |
| | | CGATGAACTTGAGAATGTCTGGCCGTTGGTGCCGCCCGGCGCCAG |
| | | TAATGCAGAAATGCTGGAGAAATTATCATGA |

### Example 10: CK1 Pathogenicity Analysis

CK1 was grown on agar plates in the presence of different antibiotics and enzyme inhibitor combinations to detect carbapenemase enzymes and evaluate CK1's pathogenicity.

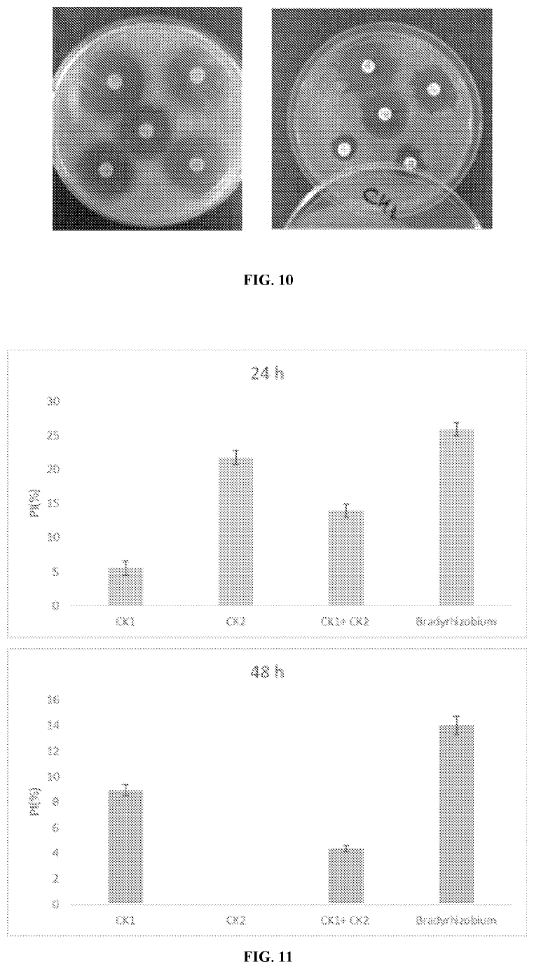

CK1 was tested using the agar diffusion method with DCM kits (Britannia) according to manufacturer's protocol. Briefly, Cultures were grown at 30° C. for 10 hours. The results showed a circular inhibition zone in the discs of the DCM kit, which indicated that CK1 is not a producer of carbapenemase type Klebsiella pneumoniae, carbapenemase (KPC), metallo-beta-lactamases (MLB), and Oxacillinase (Oxa). The results showed a lack of carbapenemase functionality and lack of pathogenicity in CK1 (FIG. 10).

### Example 11: Characterization of Antibiotic Resistance and Sensitivity

CK1 and CK2 were grown on agar plates in the presence of different antibiotics and in different concentrations to evaluate their antibiotic resistance capacity. Cultures were grown at 30° C. for 24 hours. Results are shown in Table 34 and 35 (R: resistant: S: sensitive).

TABLE 34
Bacterial Resistance to Antibiotics

| | Tetracycline | | | Kanamycin | | | Gentamicin | | | Streptomycin | | | Ampicillin | | |
|---|---|---|---|---|---|---|---|---|---|---|---|---|---|---|---|
| Concentration | 5 | 10 | 15 | 10 | 25 | 50 | 10 | 25 | 50 | 25 | 50 | 75 | 50 | 75 | 100 |
| μg/ml | | | | | | | | | | | | | | | |
| CK1 Klebsiella | R | R | R | R | S | S | R | S | S | R | S | S | R | R | R |
| aerogenes | | | | | | | | | | | | | | | |
| CK2 Bacillus | S | S | S | R | S | S | R | S | S | S | S | S | S | S | S |
| cereus | | | | | | | | | | | | | | | |

| | Chloramphenicol | | | Vancomycin | | | Carbenicillin | | | Cephalexin | | |
|---|---|---|---|---|---|---|---|---|---|---|---|---|
| Concentration μg/ml | 2 | 10 | 50 | 0.5 | 2 | 4 | 100 | 250 | 500 | 1 | 2.5 | 5 |
| CK1 Klebsiella aerogenes | R | R | S | R | R | R | S | S | S | S | S | S |
| CK2 Bacillus cereus | S | S | S | R | R | S | R | R | S | R | R | R |

CK1 and CK2 were further tested using the agar diffusion method with DCM kits (Britannia) according to manufacturer's protocol for their resistance to following antibiotics and combinations were tested using the agar diffusion method: Ciprofloxacina, Amicacina, cefepime, Tazatobatam-Piperacilina, Ceftolozane-Tazobactam, Meropenem, Meropenem-Tazobactam, Meropenem-EDTA, Meropenem-boronic acid, and Meropenem-cloxacilina.

CK1 and CK2 did not show resistance to any these antibiotics.

### Example 12: Effects of CK1 and CK2 on Fungal Growth

Bacterial strains CK1 and CK2 were tested for their ability to inhibit the growth of phytopathogenic fungi by employing a dual culture assay on PDA plates. 10 μl of bacterial cell suspension (10⁸-10⁹ CFU/mL) grown previously in LB medium was placed on one side of the Petri dishes, leaving 1 cm from the margin or alternatively as four microdroplets at each end of the Petri dish. Bradyrhizobium was used as a control. Once the moisture from the inoculum was absorbed by the culture medium, a mycelial disk (6 mm) of the phytopathogen (Fusarium sp, and Sclerotinia Sclerotium) was placed at a distance of 70 mm from the bacterial culture or alternatively the phytopathogen was placed in the middle of the four microdroplets corresponding to the bacterial culture. Plates with sterile distilled water (instead of bacterial suspension) and antagonist fungus served as control. The plates were incubated at 28+2° C. in the dark, and the percentage of inhibition (PI) was registered daily for 220 h. The PI was calculated according to Shrivastava et al. (P. Shrivastava, R. Kumar, M. S. Yandigeri, In vitro biocontrol activity of halotolerant Streptomyces aureofaciens K20: a potent antagonist against Macrophomina phaseolina (Tassi) Goid, Saudi J. Biol. Sci. 24 (2017) 192-199, https://doi.org/10.1016/j.sjbs.2015.12.004.) as follows: % PI=(C−T)/C×100 where ‘C’ is the colony growth of M. phaseolina in control plates, and T is the colony growth of the pathogen in dual-culture plates.

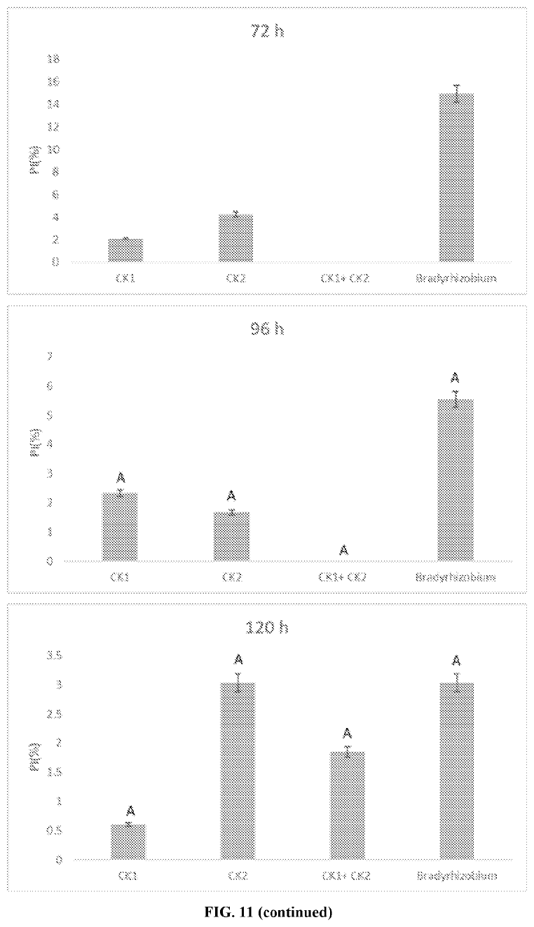

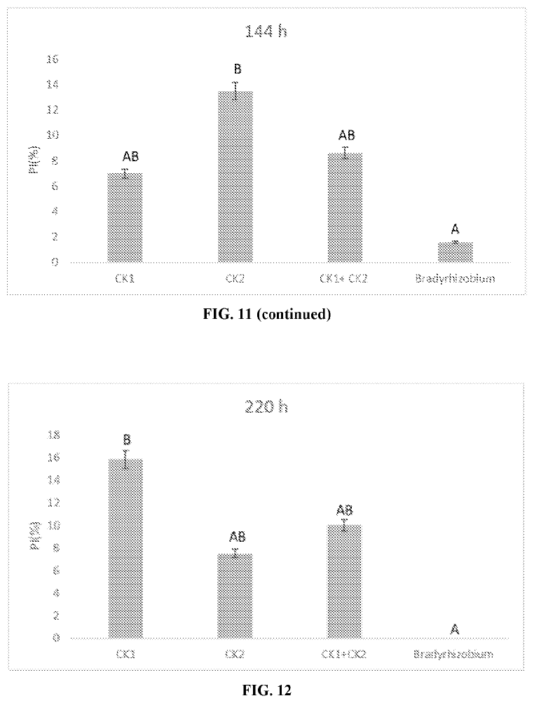

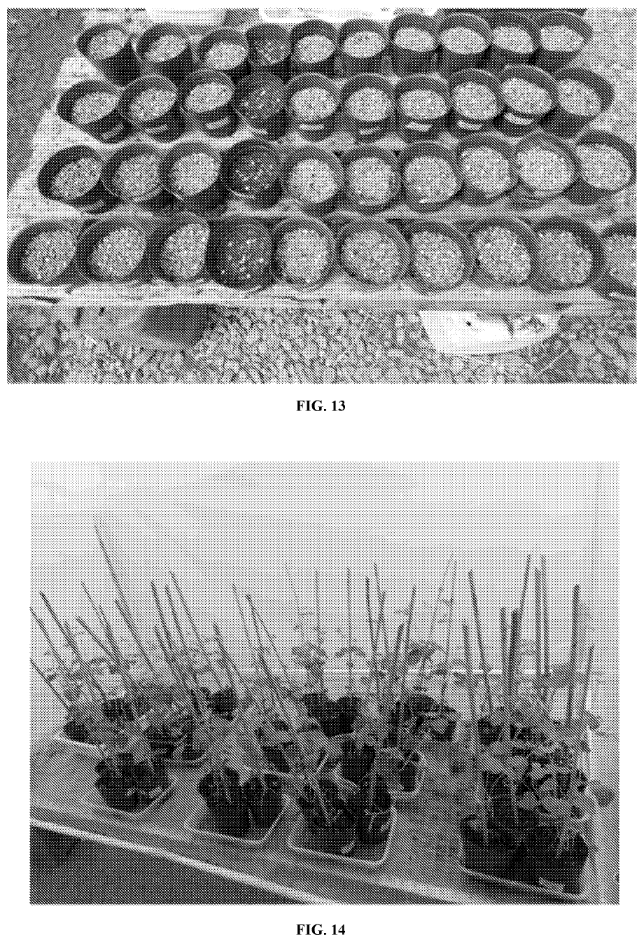

Results are shown in FIG. 11 and FIG. 12. Each bacteria and bacteria combination was differentially effective against each fungus. The assays revealed CK1 and CK2 bacteria were able to inhibit fungal growth. In general CK1. CK2 and CK1+CK2 mix were all more effective long term in inhibiting fungal growth as compared to the commercial Bradyrhizobium product

### Example 13: Ammonia and Nitrate Production and Total Nitrogen Quantification in Soybean Plants

### Ammonia and Nitrate Production

### Methodology:

The ability to synthase ammonia and nitrate was evaluated in CK1. CK2. CK1+CK2, the diazotrophic bacterium Gluconacetobacter PAL5 (a universal free nitrogen fixer) and Bradyrhizobium. All the strains were cultured in ECO culture medium (see the composition below) at 200 rpm for 24 h. After the incubation time, the commercials ab83360—Ammonia Assay Kit and Abnova™ Nitrate/Nitrite Colorimetric Assay Kit were used to assay the abilities of bacteria to synthetize Ammonia and Nitrate.

### Ammonia Detection

For the ammonia detection, the strains (previously grow in ECO medium) were centrifugated at 10,000 rpm for 10 min and washed in cold PBS buffer. Then, the cells were resuspended in 100 μL of Assay Buffer (part of the kit) and homogenized quickly by pipetting up and down a few times. The samples were centrifugated for 5 min at 4° C. at 13.000 rpm in order to remove any insoluble material. The supernatants were transferred to a clean tube and kept on ice. Finally, the supernatants were mixed with 50 μL of the reaction mix (commercialized in the kit) and incubated at 37° C. for 60 minutes (in the incubation the samples were protected from light). After the incubation, the optical density was measured at 570 nm. The production was calculated introducing the OD values obtained in a standard curve ammonia (μM).

ECO composition (g/L): 3 glucose. 5 NH4H2PO4 and 3 yeast extract PBS buffer composition (g/L): 8 NaCl. 0.2 KCl. 1.15 Na2HPO4, 0.2 KH2PO4

### Nitrate Detection

For the Nitrate detection, 80 μL of each bacteria (previously grow in ECO medium) was mixed with: 10 μL of the Enzyme Cofactor Mixture and 10 μL of the Nitrate Reductase. The samples were covered and incubated at room temperature for one hour. After the incubation time, 50 μL of Griess Reagent R1 and 50 μL of Griess Reagent R2 were added to each sample. Once the color to development appeared after an incubation of 10 minutes at room temperature, the absorbance was read at 540 nm. The production was calculated introducing the OD values obtained in a standard curve Nitrate (μM).

### Results:

TABLE 36
Ammonia and Nitrate quantification using commercial kits

| | | | Klebsiella | | |
|---|---|---|---|---|---|
| | Klebsiella | Bacillus | aerogenes CK1 + | | |
| | aerogenes | cereus | Bacillus cereus | Gluconacetobacter | |
| | CK1 | CK2 | CK2 | PAL5 | Bradyrhizobium |
| Ammonia (μM) | 89.3 | 82.76 | 117.63 | 58.7 | 24.42 |
| Nitrate (μM) | 129.07 | 108.7 | 145.0 | 114.22 | 70.56 |

Conclusion: Extremophiles microorganism were able to synthetize Ammonia and Nitrate at the same concentrations as the universal Gluconacetobacter PAL5. Also, this ability was stimulated when the strains CK1 and CK2 were combined.

Regarding to Bradyrhizobium, the strain did not show the ability to synthetize ammonium and nitrate under laboratory conditions. That's because this type of bacteria needs to establish a symbiotic relationship with the plant to fix the nitrogen.

### Total Quantification of Nitrogen in 40-Days Soybean Plants

### Methodology:

Soybean seeds were mixed with the products Extremia A, CK2+ stabilizer, Extremia MIX, and Bradyrhizobium (the preparation of the products was already described in Example 3, please noticed that CK2+ stabilizer was prepared as well as Extremia A) using a dose of 5 kg/mL for Extremia A and CK2+ stabilizer. The commercial dose for Bradyrhizobium was 0.9 kg/mL. Soybean seeds inoculated with regular water instead of biological products were used as controls. The seeds were sown in pots with fertile soil under greenhouse conditions, with an approximate temperature of 30-33° C. for 40 days. After 40 days, whole plants (leaves, roots, and stems) were dried at 80° C. for 48 hours and the total nitrogen content (%) was calculated using the Kjeldahl method.

### Kjeldahl Method

0.5 g of dried soybean tissues were introduced into a Kjeldahl tube and mixed with 5 g of catalyst regent (CuSO4+K2SO4). Then, 10 mL of H2SO4 (concentrated) was carefully added to the Kjeldahl tube and the regents (without the soybean samples were used as a blank. The samples were placed in a Kjeldahl digester and heated for approximately 90 minutes, until no release of white fumes was observed. The distillation was carried out using a Büchi automatic distiller (in the presence of NaOH) and the samples were collected in 60 mL of 4% boric acid+indicators (cresol bromine green and methyl red). Finally, a titration of the distilled samples was arrived out with H2SO4 (0.0601 N) until the color change from light blue to red. This assay was performed in duplicate. The results were expressed as a percentage (w/w).

### Results

TABLE 37
Total Nitrogen quantification in 40-days soybean plants
inoculated with extremophiles by Kjeldahl method

| | | Bacillus | | | |
|---|---|---|---|---|---|
| | Extremia | cereus CK2 + | | | |
| | A | stabilizer | Extremia MIX | Bradyrizobium | Control |
| Nitrogen (%) | 3.2 ± 0.01 c | 2.82 ± 0.001 a | 3.15 ± 0.01 c | 3.17 ± 0.02 c | 3.0 ± 0.01 b |

The values reported in the table are the averages±standard error. Different letters indicate significant differences among means (p<0.05) according to Tukey's HSD test.

Conclusion: At 40 days, the products Extremia A, MIX and Bradyrhizobium significantly enhance the total content of nitrogen in soybean plants compared to control. Additionally, these results showed that the application of Extremia A and Mix in soybean seeds improve the nitrogen content as well as the application of commercial products (Bradyrhizobium).

As it has been reported. Bradyrhizobium is a biological Nitrogen fixer that has the ability to establish a symbiosis with leguminous plants (such as soybean) by the formation of nodules in roots. This symbiosis allows the conversion of the atmospheric nitrogen (N2) into ammonium (NH4+) and make it assimilable for the plant.

Extremophiles bacteria showed a different mechanism to enhance the Nitrogen uptake that does not involve biological fixation (please, see Example 7 in the genome this pathway is absent). The products improve the nitrogen content in the plant, leaving a greater amount of nitrate and ammonium (Table 37) available for plants assimilation.

In this assay. Gluconacetobacter PAL5 was not tested because leguminous plants are not commonly inoculated with this product.

### Example 14: Organic Acids Production

### Organic Acids Production

### Methodology:

The organic acids production was detected in the supernatant of the following strains: CK1. CK2. CK1+CK2. E. coli, and Bradyrhizobium. The bacteria CK1. CK2. CK1+CK2 and E. coli were cultured in Luria Bertani (LB) medium overnight at 30° C. 200 rpm. Regarding to Bradyrhizobium, this strain was cultures in Yeast-Mannitol liquid medium for 48 h at 30° C. 200 rpm. After the incubation time, the cells were washed twice with a 0.85% (w/v) physiological solution. Then, 200 μL of each bacteria were cultured in 20 mL of NBRIP culture medium (with tricalcium phosphate (Ca3(PO4)2) as an insoluble phosphate source) and incubated at 30° C. 150 rpm for 5 days. Due to the presence of suspended particles of insoluble Ca: (PO4) 2 in the supernatant, the broths were centrifuged at 13.000 rpm for 10 min to obtain a clear supernatant. Triplicate aliquots of the supernatant (100 μl) were transferred into clean, dry, acid washed test tubes and the determination of five organic acids were carried out by High Performance Liquid Chromatography (HPLC). The experiment was performed using a C18 column (250× 4.6 mm) and the parameters were set as follows: solvent 20% methanol and 80% deionized sterilized H2O; flow rate 0.8 ml/min: temperature 40° C.: UV detector 210 nm and injection Autoclaved un-inoculated medium and E. coli inoculated media served as negative controls.

LB composition (g/L): 10 yeast extract. 5 NaCl and 10 tripteine. pH=7

Yeast-Mannitol medium composition (g/L): 5 mannitol. 0.5 yeast extract. 0.5 K2HPO4, 0.2 MgSO4 7 H2O, 0.1 NaCl. FeCl3 6 H20 (one drop of 10% solution). MnSO4 (one drop of 10% solution). 5 mL Congo Red (0.5 g+200 mL). pH 6.5-6.8

NBRIP composition (g/L): 10 Glucose. 5 Ca3(PO4)2, 5 MgCl2·6H2O, 0.25 MgSO4 7 H2O, 0.2 KCl, 0.1 (NH4)2SO4. The pH of the NBRIP medium is adjusted to 6.75±0.25 before sterilization (autoclave 121° C. 1 atm. during 15 min).

### Results:

TABLE 38
Organic Acids Production quantified by HPLC

| | | | Klebsiella | | |
|---|---|---|---|---|---|
| | | | aerogenes | | |
| Organic | Klebsiella | Bacillus | CK1 + | | |
| Acids | aerogenes | cereus | Bacillus | E. | |
| (mg/L) | CK1 | CK2 | cereus CK2 | coli | Bradyrizobium |
| Lactic Acid | 163.33 | 43.05 | 134.34 | <5 | 33.45 |
| Acetic Acid | 205.23 | <5 | 189.44 | <5 | 39.62 |
| Citric Acid | 595.54 | 61.56 | 374.14 | <5 | 24.83 |
| Malic Acid | 195.43 | 143.55 | 168.45 | <5 | 354.46 |
| Succinic | 198.26 | <5 | 95.19 | <5 | 16.52 |
| Acid | | | | | |

Conclusion: These results are complemented with Example 8, where the organic acids genes were already described.

### Inorganic Phosphate Detection:

### Methodology:

The inorganic phosphate was evaluated in the supernatant of the following strains: CK1, CK2, CK1+CK2, E. coli, and Bradyrhizobium. The bacteria CK1, CK2, CK1+CK2 and E. coli were cultured in NBRIP culture medium (see the composition above) at 200 rpm for 24 h. Then, the bacteria were centrifugated at 10,000 rpm for 10 min, and 10 μl of the supernatant was analyzed for phosphate content using a commercial kit EnzChek& Phosphate Assay Kit (E-6646). For which, the supernatant was mixed with a reaction mix, prepared as follows:
740 μL of dH2O
50 μL 20× reaction buffer
200 μL MESG substrate solution
10 μL purine nucleoside phosphorylase
10 μL experimental enzyme

Then, the samples were preincubated for 10 minutes at 22° C. Finally, the absorbance was reded at 360 nm as a function of time for both the experimental reaction and the control reaction. The production was calculated introducing the OD values obtained in a standard curve phosphate (mmolar). Results:

TABLE 39
Inorganic phosphate quantification with a commercial kit

| | | | Klebsiella | | |
|---|---|---|---|---|---|
| | | | aerogenes | | |
| | | | CK1 + | | |
| | Klebsiella | Bacillus | Bacillus | | |
| | aerogenes | cereus | cereus | | |
| | CK1 | CK2 | CK2 | E. coli | Bradyrizobium |
| Inorganic | 0.47 | 0.85 | 3.2 | 0.078 | 0.074 |
| Phosphate | | | | | |
| (mmol/UFC) | | | | | |

Conclusion: Extremophilic microorganisms were able to solubilize inorganic phosphate both individually and in combination. According to the genetic analysis of phosphate solubilization (example 8), we would expect that CK1 be a better phosphate solubilizer than CK2 due to the greater genetic diversity exhibited. However, CK2 showed a greater inorganic phosphate ability (Table 39) in the quantification with the commercial kit.

When the extremophiles were grown together this ability highly increased. This effect could be explained due to the different genomic and biochemical features exhibited by CK1 and CK2. Potentially, CK2 highly expressed the gene gdh which encodes a “glutamate dehydrogenase” (this gene is absent in CK1). In addition, CK1 could have the ability to express the gene gcd, encoding for a “glucose dehydrogenase” (absent in CK2 genome). The implication of both genes has been largely described in phosphate solubilization process. Therefore, it could be hypothesized that a beneficial trait between both strains could be responsible for the grater phosphate solubilization process.

Regarding to Bradyrhizobium, the strain showed the ability to solubilize inorganic phosphate at similar concentrations as E. coli (the strain used as negative control).

### Example: 15: Evaluation of Soybean-Seed Treatments in the Control of Fusarium tucumaniae in Greenhouse Trials

### Methodology

The trial was planted on Feb. 24, 2022, in the Phytopathology laboratory of the EEAOC, Las Talitas, Tucumán, Argentina. The soybean variety used was DM 5958. The design experiment was completely randomized with four repetitions (FIG. 14). Each experimental unit consisted of a plastic pot (10 cm diameter×13 cm) with 100 g of the sterile substrate (Grow Mix Multipro) sown with 6 soybean seeds. Those treatments that included the pathogen were inoculated at the time of sowing with 15 g of sorghum inoculated with F. tucumaniae. The trial included 10 treatments (Table 40).

The parameters evaluated were the following:
Plant emergence at 7, 10, 14, and 20 days after sowing
Severity at the root (scale from 1 to 5)
Fresh weight (g)

The results obtained from the tests were statistically analyzed using the Infostat program (Di Renzo et al., 2008). The emergence parameter of plants was evaluated using mixed generalized linear models (MLGM) and a test comparison of means (LSD, α=0.05). The root severity and fresh weight parameters were statistically evaluated with general and mixed linear models and a mean comparison test (LSD, α=0.05).

TABLE 40
Treatments and doses of seed treatments applied in the greenhouse trials.
System: soybean/Fusarium tucumaniae

| | Doses |
|---|---|
| | (ml/100 kg |
| Treatments | seeds) |
| 1- Klebsiella aerogenes CK1 + Bacillus cereus CK2 + | 500 |
| Exiguobacterium undeae CK3 | |
| 2- Klebsiella aerogenes CK1 + Bacillus cereus CK2 + | 300 |
| Exiguobacterium undeae CK3 | |
| 3- Klebsiella aerogenes CK1 + Bacillus cereus CK2 | 500 |
| 4- Klebsiella aerogenes CK1 + Bacillus cereus CK2 | 300 |
| 5- Bacillus cereus CK2 + Exiguobacterium undeae CK3 | 500 |
| 6- Bacillus cereus CK2 + Exiguobacterium undeae CK3 | 300 |
| 7-Control no pathogen | — |
| 8-Control pathogen | — |
| 9-Control biofungicide (Rizoderma) | 300 |

### Results

The results of the greenhouse trials against F. tucumaniae in soybeans are presented in Tables 41 and 42.

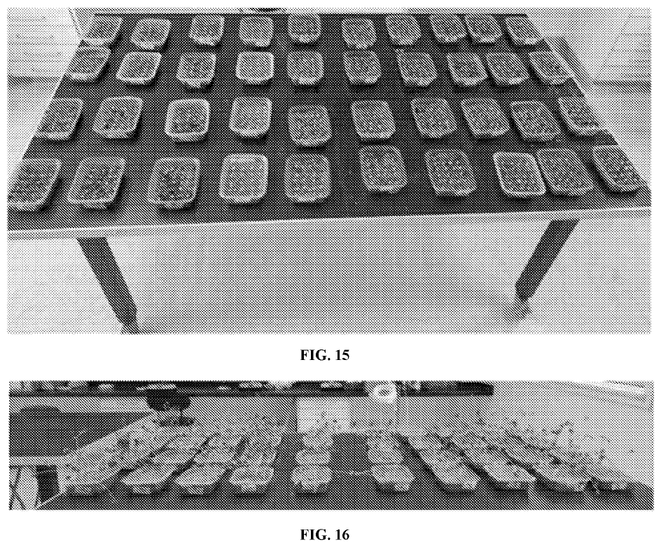

For the variable plant emergence (Table 41), no significant differences were observed between the treatments evaluated at 7 days after sowing (dds) (P=0.3811). No significant differences were observed between the evaluated treatments at 10 and 14 days (P=0.5599 and P=0.4722). The control without pathogen presented an emergence value of 75.0% at 10 days and 79.2% at 14 days and the pathogen control showed a value of 66.7% and 62.5% respectively. Among treatments inoculated with F. tucumaniae, the one that presented the highest values of emergence on both dates was treatment 10 (91.7%) followed by treatment 5 (79.2%) and treatment 4 (75.0%). At 20 days, no significant differences were observed among evaluated treatments (P=0.3277). Among the treatments inoculated with the pathogen that presented the highest emergence values, could be highlighted treatment 10 (91.7%) followed by treatments 5 (79.2%) and 4 (75.0%) (FIG. 15).

TABLE 41
Average of plants number and emergence (%) in soybean (DM
5958) infected with F. tucumaniae in a greenhouse trial.

| | Emergence | | | | | | | |
|---|---|---|---|---|---|---|---|---|
| System: soybean/Fusarium tucumaniae | 7 days | | 10 days | | 14 days | | 20 days | |
| Treatments | No | % | No | % | No | % | No | % |
| 1- Klebsiella aerogenes CK1 + Bacillus | 3.7 | 62.5 | 4.25 | 70.8 | 4.50 | 75 | 4.25 | 70.8 |
| cereus CK2 + Exiguobacterium undeae | | | | | | | | |
| CK3 | | | | | | | | |
| 2- Klebsiella aerogenes CK1 + Bacillus | 4 | 66.7 | 4.25 | 70.8 | 4.25 | 70.8 | 4.25 | 70.8 |
| cereus CK2 + Exiguobacterium undeae | | | | | | | | |
| CK3 | | | | | | | | |
| 3- Klebsiella aerogenes CK1 + Bacillus | 3 | 50 | 3.50 | 58.3 | 3.50 | 58.3 | 3.25 | 54.2 |
| cereus CK2 | | | | | | | | |
| 4- Klebsiella aerogenes CK1 + Bacillus | 4.25 | 70.8 | 4.50 | 75 | 4.50 | 75 | 4.50 | 75 |
| cereus CK2 | | | | | | | | |
| 5- Bacillus cereus CK2 + | 4 | 66.7 | 4.75 | 79.2 | 4.75 | 79.2 | 4.75 | 79.2 |
| Exiguobacterium undeae CK3 | | | | | | | | |
| 6- Bacillus cereus CK2 + | 4 | 66.7 | 4 | 66.7 | 4.25 | 70.8 | 4 | 66.7 |
| Exiguobacterium undeae CK3 | | | | | | | | |
| 7-Control no pathogen | 4.50 | 75 | 4.50 | 75 | 4.75 | 79.2 | 4.75 | 79.2 |
| 8-Control pathogen | 3.50 | 58.3 | 4 | 66.7 | 3.75 | 62.5 | 3.75 | 62.5 |
| 9-Control biofungicide (Rizoderma) | 4 | 66.7 | 4 | 66.7 | 4 | 66.7 | 3.75 | 62.5 |
| 10-Control chemical (Acronis: | 5.50 | 91.7 | 5.50 | 91.7 | 5.50 | 91.7 | 5.50 | 91.7 |
| pyraclostrobin + methylthiophanate) | | | | | | | | |
| P-value | 0.3811 | | 0.5599 | | 0.4722 | | 0.3277 | |

*Means in each column followed by the same letter do not differ significantly (LSD, P < 0.05).

Regarding the root severity, the pathogen control presented values of 2.21. All seed treatments inoculated with the pathogen were statistically different (P<0.0001) from the pathogen control, being the treatments 10, 9, 6, 2, 4, and 5 the ones that presented the lowest values (Table 42). For the fresh weight measurement, no treatment was statistically different from the pathogen control (7.8 g) (P=0.0810) (Table 42).

TABLE 42
Root severity and fresh weight in soybean (DM 5958)
infected with F. tucumaniae in a greenhouse trial.
System: Soybean/Fusarium tucumaniae

| | | | Weight |
|---|---|---|---|
| Treatments | Severity | | (g) |
| 1- Klebsiella aerogenes CK1 + Bacillus cereus | 1.23 | B | 9.3 |
| CK2 + Exiguobacterium undeae CK3 | | | |
| 2- Klebsiella aerogenes CK1 + Bacillus cereus | 0.68 | CD | 9.5 |
| CK2 + Exiguobacterium undeae CK3 | | | |
| 3- Klebsiella aerogenes CK1 + Bacillus cereus CK2 | 0.96 | BC | 6.4 |
| 4- Klebsiella aerogenes CK1 + Bacillus cereus CK2 | 0.73 | BCD | 10.3 |
| 5- Bacillus cereus CK2 + Exiguobacterium undeae CK3 | 0.80 | BCD | 9.2 |
| 6- Bacillus cereus CK2 + Exiguobacterium undeae CK3 | 0.65 | CD | 10 |
| 7-Control no pathogen | 0.43 | D | 9.2 |
| 8-Control pathogen | 2.21 | A | 7.8 |
| 9-Control biofungicide (Rizoderma) | 0.65 | CD | 8.1 |
| 10-Control chemical (Acronis: pyraclostrobin + | 0.41 | D | 12.9 |
| methylthiophanate) | | | |

| | | |
|---|---|---|
| P-value | <0.0001 | 0.0810 |

*Means in each column followed by the same letter do not differ significantly (LSD, P < 0.05).

### Conclusions

Inoculations with F. tucumaniae under controlled conditions were effective in the test carried out on soybeans (DM5958). However, the values of plant emergence in soybean were not affected when inoculated with F. tucumaniae.

The pathogen control presented the highest severity value in the roots. The rest of the treatments were statistically different (P<0.0001) from the pathogen control.

The fresh weight of soybean plants was affected by the presence of F. tucumaniae. In addition, the rest of the evaluated treatments were not statistically different from the pathogen control (P=0.0810).

### BIBLIOGRAPHY

Di Rienzo J. A., Casanoves F., Balzarini M. G., Gonzalez L., Tablada M., Robledo C. W. 2008. InfoStat, versión 2008, Grupo InfoStat, FCA, Universidad Nacional de Córdoba, Argentina.

### Example 16: Evaluation of Soybean-Seed Treatments in the Control of Macrophomina phaseolina in Greenhouse Trials

The trial was planted on Feb. 1, 2022, in the Phytopathology laboratory of the EEAOC, Las Talitas, Tucumán, Argentina. The soybean variety used was M6410 IPRO. The design experiment was completely randomized with four repetitions (FIG. 16). Each experimental unit consisted of a plastic pot (12×16×5 cm) with 700 g of sterile sand sown with 20 soybean seeds. Those treatments that included the pathogen were inoculated at the time of sowing with 2.5% p/p of rice inoculated with M. phaseolina. The trial included 10 treatments (Table 43).

The parameters evaluated were the following:
Plant emergence at 3, 6, and 10 days after sowing
Plant height (cm)
Fresh weight (g)
Severity at the root (scale from 1 to 5)

The results obtained from the tests were statistically analyzed using the Infostat program (Di Renzo et al., 2008). The emergence parameter of plants was evaluated by means of mixed generalized linear models (MLGM) and a test comparison of means (LSD, α=0.05). The plant height, root severity, and fresh weight parameters were statistically evaluated with general and mixed linear models and a mean comparison test (LSD, α=0.05).

TABLE 43
Treatments and doses of seed treatments applied in the greenhouse trials.
System: soybean/Macrophomina phaseolina

| | Doses |
|---|---|
| | (mL/100 Kg |
| Treatments | of seeds) |
| 1- Klebsiella aerogenes CK1 + Bacillus cereus CK2 + | 500 |
| Exiguobacterium undeae CK3 | |
| 2- Klebsiella aerogenes CK1 + Bacillus cereus CK2 + | 300 |
| Exiguobacterium undeae CK3 | |
| 3- Klebsiella aerogenes CK1 + Bacillus cereus CK2 | 500 |
| 4- Klebsiella aerogenes CK1 + Bacillus cereus CK2 | 300 |
| 5- Bacillus cereus CK2 + Exiguobacterium undeae CK3 | 500 |
| 6- Bacillus cereus CK2 + Exiguobacterium undeae CK3 | 300 |
| 7-Control no pathogen | — |
| 8-Control pathogen | — |
| 9-Control biofungicide (Rizoderma) | 300 |
| 10-Control chemical (Acronis: pyraclostrobin + | 100 |
| methylthiophanate) | |

### Results

The results of the greenhouse trials against M. phaseolina in soybeans are presented in Tables 44 and 45.

For the plant emergence measurement (Table 44), no significant differences were observed among the evaluated treatments at 3 days after sowing (dds) (P>0.9999).

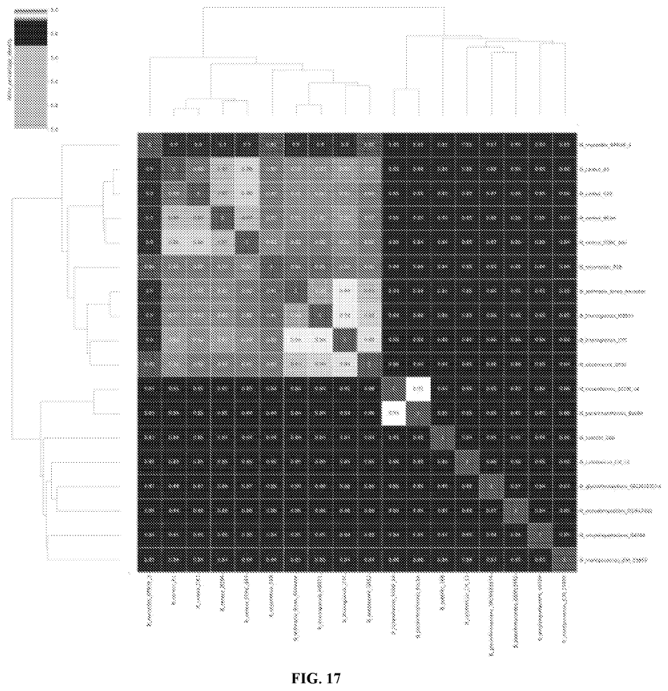

At 6 dds, significant differences were shown among the treatments (P<0.0001). The control without pathogen presented an emergence value of 87.50% being statistically different from the rest of the treatments except for treatment 10 (Acronis) which presented a value of 77.50%. The highest emergence values obtained belonged to treatment 10 (77.50%) followed by treatments 1, 2, and 3 with an emergency of 42.50%. At 10 days, the seed treatments that presented the highest emergency values were treatment 10 (75.00%) followed by treatments 3, 5, and 9 (55.00%), presenting statistical differences compared to the pathogen control (28.35%) (FIG. 17).

TABLE 44
Average of plants number and emergence (%) in soybean (M6410
IPRO) infected with M. phaseolina in a greenhouse trial.

| System: soybean/Macrophomina | | | | | | |
|---|---|---|---|---|---|---|
| phaseolina | Emergence | | | | | |
| Treatments | No | % | No | % | No | % |
| 1- Klebsiella aerogenes CK1 + Bacillus | 2.75 | 13.75 | 8.50 B | 42.50 | 10.33 C | 51.65 |
| cereus CK2 + Exiguobacterium undeae CK3 | | | | | | |
| 2- Klebsiella aerogenes CK1 + Bacillus | 2.50 | 12.50 | 8.50 B | 42.50 | 9 CD | 45 |
| cereus CK2 + Exiguobacterium undeae CK3 | | | | | | |
| 3- Klebsiella aerogenes CK1 + Bacillus | 3.25 | 16.25 | 8.50 B | 42.50 | 11 C | 55 |
| cereus CK2 | | | | | | |
| 4- Klebsiella aerogenes CK1 + Bacillus | 0.75 | 3.75 | 5 C | 25 | 7.66 CD | 38.30 |
| cereus CK2 | | | | | | |
| 5- Bacillus cereus CK2 + Exiguobacterium | 1.75 | 8.75 | 7 BC | 35 | 11 C | 55 |
| undeae CK3 | | | | | | |
| 6- Bacillus cereus CK2 + Exiguobacterium | 0.75 | 3.75 | 7.25 BC | 36.25 | 8.25 CD | 41.25 |
| undeae CK3 | | | | | | |
| 7-Control no pathogen | 13.25 | 66.25 | 17.50 A | 87.50 | 18.5 A | 92.50 |
| 8-Control pathogen | 1.50 | 7.50 | 7.50 BC | 37.50 | 5.67 D | 28.35 |
| 9-Control biofungicide (Rizoderma) | 0 | 0 | 6.50 BC | 32.50 | 11 C | 55 |
| 10-Control chemical (Acronis: | 3.25 | 16.25 | 15.50 A | 77.50 | 15 B | 75 |
| pyraclostrobin + methylthiophanate) | | | | | | |
| P-value | >0.9999 | | <0.0001 | | <0.0001 | |

*Means in each column followed by the same letter do not differ significantly (LSD, P < 0.05).

Regarding the root severity, the pathogen control presented values of 7.33%. All the seed treatments inoculated with the pathogen were statistically different (P<0.0001) from the pathogen control, except for treatment 2 (6.03%) (Table 45).

For the fresh weight measurement, no treatment was statistically different from the pathogen control (7.63 g), except for the treatment 10 (19.43 g) and the control without the pathogen (30.07 g) (P<0.0001) (Table 45).

Regarding the stem length, statistical differences were obtained among the treatments (P<0.0001) (Table 45). Treatment 1 (11.9 cm) statistically differed from the pathogen control (P<0.0001) that showed a plant-length average of 14.37 cm.

Treatments that presented length values higher than the pathogen control were numbers 5 (15.60 cm) and 10 (16.66 cm).

TABLE 45
Root severity, fresh weight, and length measurements in soybean
(M6410 IPRO) infected with M. phaseolina in a greenhouse trial.
System: Soybean/Macrophomina phaseolina

| Treatments | Severity (%) | | Weight (g) | | Length (cm) | |
|---|---|---|---|---|---|---|
| 1- Klebsiella aerogenes CK1 + Bacillus cereus CK2 + | 3.33 | B | 10.67 | C | 11.19 | D |
| Exiguobacterium undeae CK3 | | | | | | |
| 2- Klebsiella aerogenes CK1 + Bacillus cereus CK2 + | 6.03 | A | 10.53 | C | 11.66 | CD |
| Exiguobacterium undeae CK3 | | | | | | |
| 3- Klebsiella aerogenes CK1 + Bacillus cereus CK2 | 1.67 | BC | 11.53 | C | 12.21 | BCD |
| 4- Klebsiella aerogenes CK1 + Bacillus cereus CK2 | 1 | BC | 7.70 | C | 12.18 | BCD |
| 5- Bacillus cereus CK2 + Exiguobacterium undeae CK3 | 0.83 | BC | 11.35 | C | 15.60 | AB |
| 6- Bacillus cereus CK2 + Exiguobacterium undeae CK3 | 2.12 | BC | 7.18 | C | 14.25 | ABC |
| 7-Control no pathogen | 0 | C | 30.07 | A | 14.34 | ABC |
| 8-Control pathogen | 7.33 | A | 7.63 | C | 14.37 | ABC |
| 9-Control biofungicide (Rizoderma) | 1.85 | BC | 11.95 | C | 14 | BCD |
| 10-Control chemical (Acronis: pyraclostrobin + | 1.95 | BC | 19.43 | B | 16.66 | A |

| | | | |
|---|---|---|---|
| methylthiophanate) | | | |
| P-value | <0.0001 | <0.0001 | 0.0002 |

*Means in each column followed by the same letter do not differ significantly (LSD, P < 0.05).

### Conclusions

In greenhouse trials, infections performed on soybeans (M6410 IPRO) with M. phaseolina were effective in causing a reduction in the plant emergence values. Seed treatments evaluated were effective in increasing the emergence values, being treatment 10 the one that presented the highest values, followed by treatments 9, 5, and 3.

Regarding the severity measurement, the pathogen control showed the highest values, while all seed treatments were statistically different (P<0.0001) from the pathogen control, except for treatment 2.

Infections with M. phaseolina reduced the fresh weight and the treatments were not statistically different from the pathogen control, except for treatment 10 (19.43 g) and the control without pathogen (30.07 g) (P<0.0001).

Infections with M. phaseolina affected plants' length but no statistical differences were observed compared to controls. The treatments that presented length values greater than the pathogen control (14.37 cm) were treatment 5 (15.60 cm) and treatment 10 (16.66 cm).

### BIBLIOGRAPHY

Di Rienzo J. A., Casanoves F., Balzarini M. G., Gonzalez L., Tablada M., Robledo C. W. 2008. InfoStat, versión 2008, Grupo InfoStat, FCA, Universidad Nacional de Córdoba, Argentina.

### Example 17: CK2 Strain Species Determination

A partial nucleotide sequence of the 16S rRNA gene (1288 nt) was obtained from the whole genome sequencing of CK2 strain and was used to search for similar sequences in the nucleotide collection (nr/nt) database from NCBI using BLAST. The results indicates that this strain belongs to the Bacillus genus.

### Average Nucleotide Identity Analysis

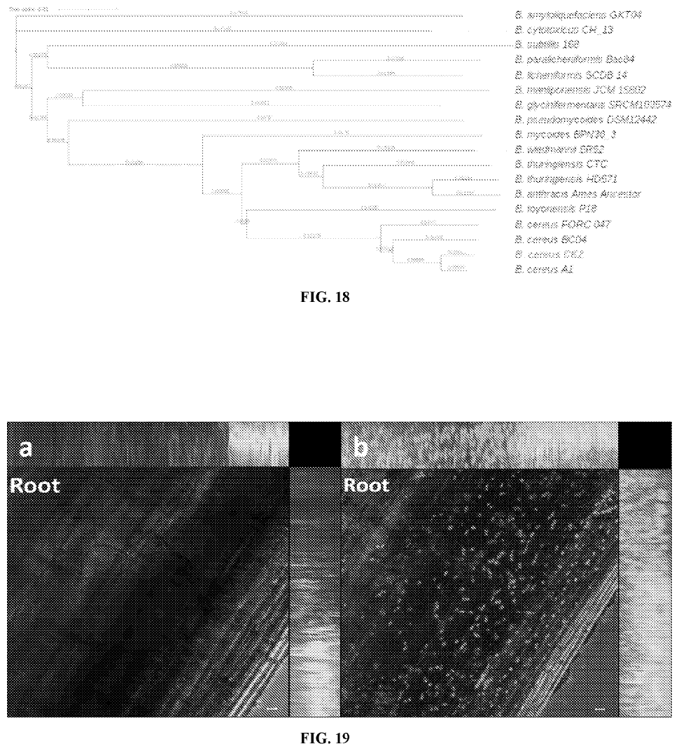

In order to identify the species of CK2 strain, an Average Nucleotide Identity (ANI) analysis with members of Bacillus genus and CK2 genome was performed. The results indicates that this strain belongs to B. cereus group species (FIG. 17, FIG. 18).

Multi Locus Sequence Typing and panC Gene Phylogenetic Analysis

Members of B. cereus group includes several species with closely related phylogeny, so in order to correctly identify the specific clade that this strain belongs to, two sequence analyses were performed: an analysis using the Multi Locus Sequence Typing (MLST) from PubMLST and an analysis of the panC gene.

All genes of the MLST found in this strain have 100% identity with strains included in clonal complex ST-18 (B. cereus sensu stricto) of PubMLST typing schema. The phylogenetic analysis of panC gene sequence positions this strain in Group IV (B. cereus sensu stricto). Overall, these results indicates that this strain belongs to the species B. cereus sensu stricto.

### Example 18: Expression of Green Fluorescent (GFP) in CK1

Spontaneous mutant of CK1 resistant to rifampicin: CK1 was cultured in LB liquid medium for 24 h at 30° C., and 250 rpm. After the incubation, the cell density was adjusted to an OD (600 nm)=1 and 100 μL of CK1 was cultured in agar plates (previously prepared with LB medium supplemented with 100 μg mL⁻¹ of rifampicin). The plates were incubated for 24-48 h in the absence of light at 30° C. until colonies appeared. Finally, CK1 was re-cultured in LB agar plates with a concentration of 50 ug mL⁻¹ of rifampicin. The spontaneous mutants of CK1 resistant to rifampicin were stored at −70° C.

Rifampicin preparation: Rifampicin was weighed in laminar flow trying to maintain sterility, and then dissolved in pure methanol to obtain a final concentration of 50 μg/mL. Two drops of NaOH 10 M were also added to the complete dissolution of the antibiotic.

### Bacteria Conjugations

The conjugations performed were four-parent with the following bacteria:
Helper E. coli 2013 is kanamycin resistant, and the bacteria has the conjugation gen in the plasmid.
Transposase E. coli S17.1 pUX-BFI is ampicillin resistant.
Green fluorescent E. coli XL1-Blue pBK-miniTN7-gfp3 is kanamycin resistant.
CK1 with the spontaneous mutant to rifampicin.

The bacteria were cultured in different LB agar plates with the addition of each resistant antibiotic in a final concentration of 50 μg/mL, and incubated overnight at 30° C. After 24 h of incubation, the four bacteria were mixed for conjugation in LB agar plate (without antibiotics) for 24 h at 30° C. Once the conjugation takes place and the bacteria were re-cultured in a 2ii solid medium with the addition of rifampicin and kanamycin to allow the growth of CK1 with the expression of GFP.

Medium 2ii composition g/L: 0.125 K2HPO4, 4 (NH4)2S04, 7.5 sodium citrate, 0.2 MgSO4, 15 agar·pH=8.

Ampicillin and Kanamycin preparation: The ampicillin and kanamycin were weighed in laminar flow trying to maintain sterility, and then dissolved in sterile distilled water to obtain a final concentration of 50 μg/mL.

### Production of Extremia Mix-GFP

CK1-GFP bacterial growth: The strain (previously cultured overnight in LB broth medium with kanamycin) was re-cultured in falcons (50 mL of capacity) containing 10 mL of LB medium with the addition of kanamycin at 30° C. 230 rpm for 24 h. The morphology of the bacteria was controlled under the microscope and with Gram's method, in order to check their purity over time.

Xanthan gum preparation: The Xanthan gum was added under constant stirring and in a rain form in order to avoid conglomerates to a physiological solution (0.8 w/v) in a final concentration of 2% (w/v). Then, an antifoam was added to the process in a proportion 1:1000. Finally, all the solution was sterilized at 121° C. for 45 minutes.

Extremia mix preparation: In this step CK1 and the Xanthan gum were mixed in the following proportion: 75:25 (v/v) in order to obtain a final concentration of the gum 0.5% (v/v). Inoculation of soybean seeds with Extremix-GFP

200 g of soybean seeds were weighed in polyethylene bags and inoculated with 1 mL Extremia-GFP (dose of 5 kg/mL). Then, the bags were homogenized carefully and let the seeds dry for 10 min. Next, plastic trays were filled with sterile sand (121° C. 1 atm for 15 minutes) and the seeds were sowed (20 per tray). The seeds were irrigated with distilled sterile water and the trays were covered with plastic bags to keep them moist until the germination of seeds (2-3 days). The trays were incubated at 30° C. for a week and irrigated every 48 h (once the seeds germinate, the plastic was removed, and the incubation continues).

After a week of incubation, the plants were carefully removed and prepared to be observed in the Confocal Laser Scanning Microscope (LCSM). For which, the roots, stems (corresponding to the 1st, 2nd and 3rd portion) and leaves were cut with a scalpel. The plant tissues were placed on a slide with drops of agarose (0.8 w/v) previously tempered. A coverslip was placed on top until they dry. Finally, the samples were placed in the refrigerator until they were observed in the LCSM.

### Results:

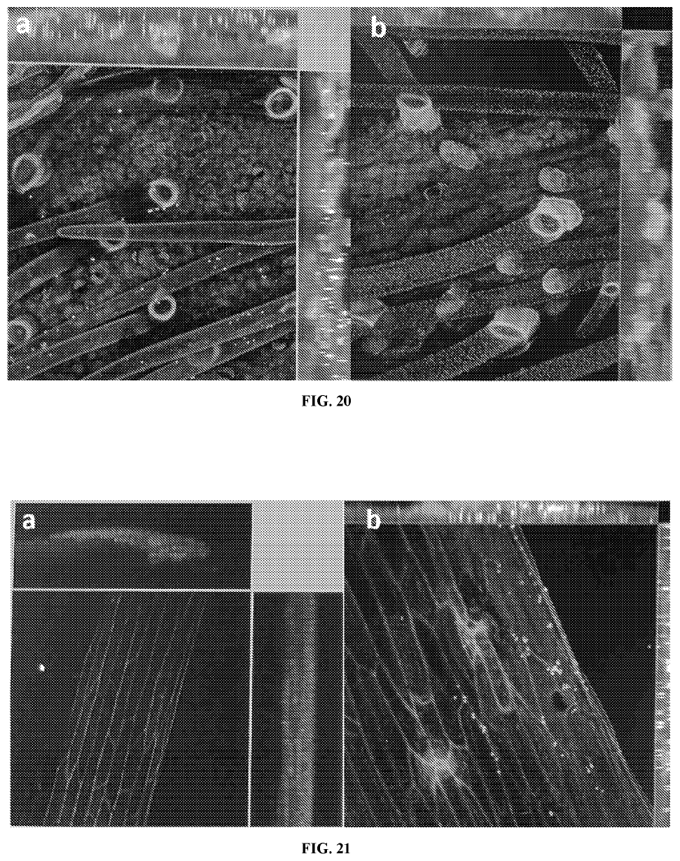

After 7 days of growth. CK1-GFP was found in the simple leaves, in the three portions of the stems (FIG. 19. FIG. 20, FIG. 21).

### Example 19: Purification and Structural Characterization of CK1 Exopolysaccharides (EPS)

### EPS Purification

The EPS extract was resuspended in doubly distilled water (H2Odd), sonicated, and dialyzed for 16 h (3500 Mw cut-off) against H2Odd. Anion exchange chromatography was then performed on a DE52 column (Whatman. 4057200). The resin was regenerated according to the manufacturer's protocol and the procedure was performed in batch. The fractions were eluted with NaCl (0-0.5 M). The total carbohydrate content in the different fractions was evaluated using the phenol-sulfuric acid method. The presence of DNA and proteins in each fraction was determined by measuring absorbance at 260 and 280 nm, respectively. The fractions containing carbohydrates were collected, dialyzed, and lyophilized for further analysis.

### MRI Analysis

The samples of EPS (˜ 50 mg) were dissolved in 500 μL of D2O and measured in a nuclear magnetic resonance (NMR) spectrometer advance III 600 MHZ (Bruker, Germany). Unidimensional 1H and two-dimensional spectra 1H-13C heteronuclear single quantum coherence (HSQC), 1H-1H total correlated spectroscopy (TOCSY). 1H-13C heteronuclear multiple bond correlation (HMBC) and 1H-1H nuclear overhauled effect spectroscopy (NOESY). To analyze the composition of monosaccharides samples were previously hydrolyzed with TFA 2 N for 2 h at 100° C. (2). The hydrolysate was dried in a lyophilizer and finally resuspended in 500 mL of sodium phosphate buffer (100 mM dissolved in D2O pH: 7.4), supplemented with 3-trimethylsilyl-[2.2.3.3,-2H4]-propionate (TSP) (final concentration 0.33 mM), as a reference of chemical displacement, for NMR analysis.

### Determination of Neutral and Acidic Sugars by HPAEC-PAD

The samples were diluted in 200 μL of H2Odd. They were then hydrolyzed. The hydrolysate was dried in a lyophilizer and resuspended in 300 μL of H2Odd. A high-performance anion exchange chromatography (HPAEC-PAD) was then performed on a Bio LC DX-3000 (Dionex) device using an amperometric pulse detector using the Carbopac P20 column with P20 pre-column (Dionex). For the determination of neutral sugars and amino sugars. NaOH 200 mM was used as solvent A and H2Odd was used as solvent B. An isocratic program was performed in 8% A and 92% B. For the determination of acidic sugars NaOH 200 mM was used as solvent A. H2Odd was used as solvent B and IM NaAcO was used as solvent C. An isocratic program was performed in 25% A. 10% C. 65% B at a flow of 0.4 mL/min.

D-glucosamine (Sigma), L-fucose (Sigma), D-mannose (Sigma), D-galactose (Sigma), D-galacturonic acid (Sigma), D-glucuronic acid (Sigma), sialic acid (Sigma), D-glucose (Merck), and an acid-containing alginate hydrolysate were used as standards. Alternatively, D-manuronic acid and D-guluronic acid were used as standards. The sugars were dried in a vacuum desiccator for 48 h. The dry cores were weighed to prepare solutions from which the necessary dilutions for analysis were made. The solutions were stored at −20° C.

### Analysis by Gas Chromatography Coupled to Mass Spectrometry (GC-MS)

The polysaccharide was methylated by the NaOH/CH 3I method. The sample (˜5 mg) was dissolved in dimethyl sulfoxide (0.5 mL), fine NaOH powder (20 mg), and methyl iodide (0.1 mL). The mixture was stirred for 6 minutes in a closed tube at 25° C. Then, H2Odd (1 mL) and chloroform (1 mL) were added. The chloroformic fas was isolated and washed with H2Odd (3×10 mL). It eventually evaporated to dryness. The methylated polysaccharide was hydrolyzed by the procedure described above. The resulting O-methylated monosaccharides were reduced to the corresponding alditols by dissolving them in 95% ethanol and adding aqueous NaBD4 (in 1 M NH4OH) for 2 h at room temperature. An acetic acid/methanol solution (1:9 v/v) was then added and evaporated at room temperature. Three washes and evaporations with acetic acid/methanol 1:9 (V/V) and two washes and evaporations with methanol were performed. The O-methylated alditols were O-acetylated with acetic anhydride for 3 h at 100° C. Finally. O-methylated and O-acetylated alditols were extracted in methylene chloride and evaporated to dryness. The products were analyzed by GC-MS with a 30 m HP5 column in splitless mode (HP). The following temperature schedule was followed: two minutes at 60° C. then an increase to 120° C. (10° C./min) holding for 2 min at 120° C. then an increase up to 280° C. (3° C./min) and finally remained at 280° C. for 5 min. Mass spectra were recorded continuously by scanning from 40 to 1000 m/z. The operating parameters of the MS were: ionization voltage of 70 eV, electron multiplier energy of 1600 V, and ion source temperature of 200° C.

### Results

### EPS Purification

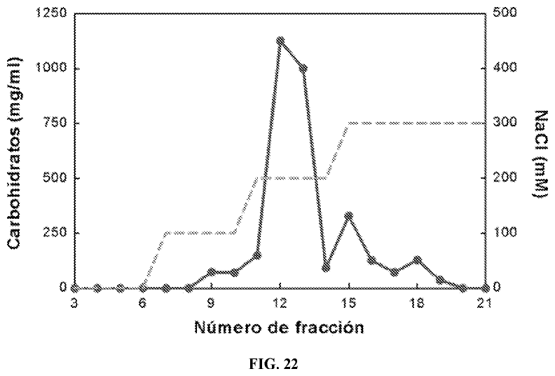

As a result of anion exchange chromatography, the profile of FIG. 22 was obtained. A peak of the majority carbohydrate concentration was observed at 200 mM NaCl (fractions 11 to 14). The purification was carried out in triplicate, obtaining in all cases similar results. Fractions 11 to 14 of the three trials were combined to continue further analysis (purified EPS).

### Determination of the Composition of Monosaccharides

### MRI Analysis

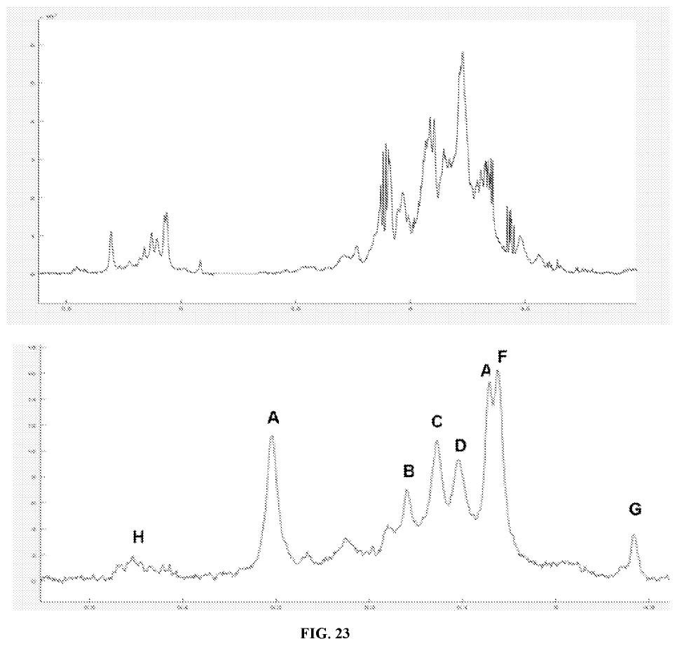

A ¹H NMR spectrum was performed on an aliquot portion of purified EPS (FIG. 23). As a result, eight resonances belonging to anomeric protons could be identified. (FIG. 23, signals A-H). This result indicates that the EPS is made up of at least eight residues of sugars different or in different conformations.

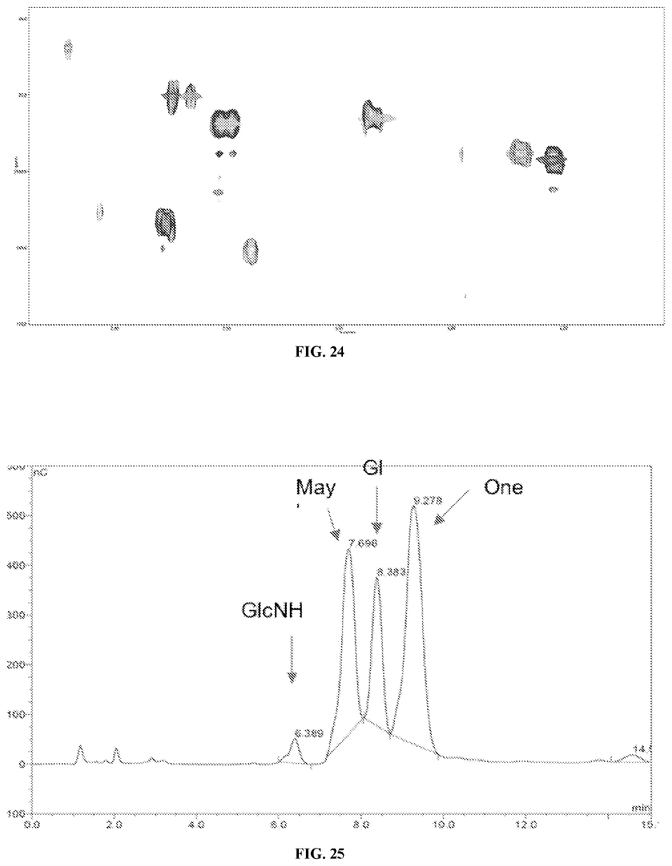

In order to determine the identity of the monosaccharides constituting EPS, a spectrum ¹H-13 C-HSQC was performed from a previously hydrolyzed sample (FIG. 24). From the spectrum obtained, and by comparison with public databases, the following monosaccharides were identified: D-Glucose, D-Mannose, D-Galactose, and D-Glucosamine. To confirm the assignment, ¹H-¹³C-HSQC spectra were performed with the corresponding patterns (FIG. 24). The results confirmed the identity of the monosaccharides found. Table 47 shows the relationship of areas between the anomeric gates of each residue.

TABLE 47
Area Ratio between anomeric protons identified by ¹H NMR

| Residue | Area Relationship |
|---|---|
| A | 5 |
| B | 5 |
| C | 5 |
| D | 5 |
| And | 5 |
| F | 5 |
| G | 1 |
| H | 1 |

### Analysis by HPAEC-PAD

### Neutral Sugars and Amino Sugars

In order to confirm the previous results, an analysis of the composition of monosaccharides by HPAEC-PAD was performed. An aliquot of the purified EPS was subjected to hydrolysis, as previously described, and diluted 1:5. The following were used as witnesses: D-Fucose (Fuc, 3.16 min): D-Glucosamine (GlcNH2, 6.21 min): D-Galactose (Gal, 7.60 min): D-Glucose (Glc, 8.25 min) and D-Mannose (Man, 9.25 min) (FIG. 25).

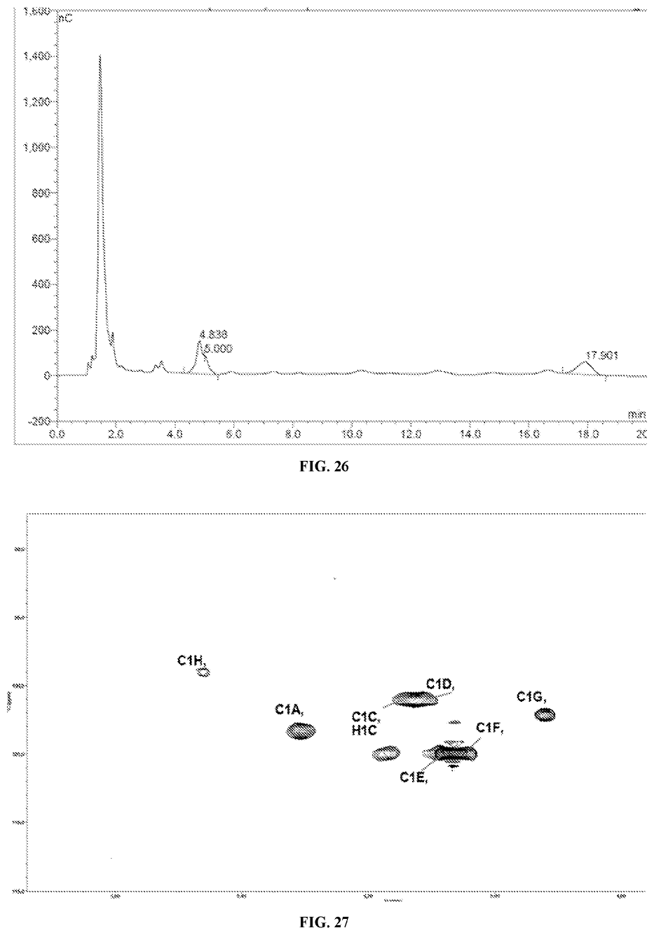

Additionally, the presence of acidic sugars was analyzed (FIG. 26). The results did not show the presence of any acidic monosaccharide. The following were used as witnesses: ac. sialic 3.35 min: GalA 11.50 min: GulA 12.60 min: GlcA 15.20 min: ManA 16.86 min.

As a result of this analysis, it was confirmed that the EPS sample showed the presence of D-Glucosamine, D-Galactose, D-Glucose and D-Mannose in a ratio of 1:10:6:16, respectively. No acid sugars were identified among the components of EPS.

### Structural Analysis of EPS

### MRI Analysis

In order to characterize the structure of the EPS and determine the sequence, anomeric configurations and ramifications, the following NMR experiments were performed: ¹H-¹³C-HSQC, ¹H-¹H-TOCSY, ¹H-¹³C-HMBC, and ¹H-¹H-NOESY.

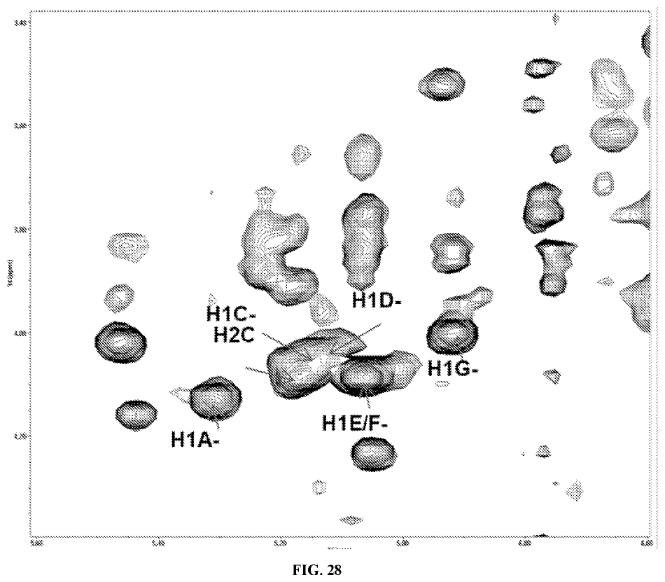

FIG. 27 illustrates a region of spectro ¹H-¹³C-HSQC of purified EPS where the resonances of ¹H and ¹³C anomeric are appreciated. FIG. 28 shows a region of spectrum 1H-1H-TOCSY (mixing time 200 ms), and FIG. 29 shows the corresponding region of spectrum 1H-1H-NOESY (mixing time 300 ms). From these spectra it was possible to determine the sequence and branching of the EPS resulting in the following connectivity:

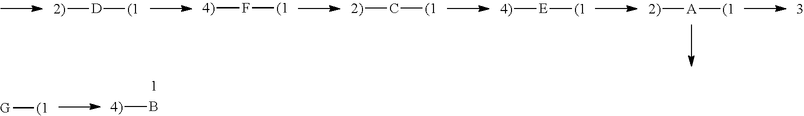

### Analysis by GC-MS

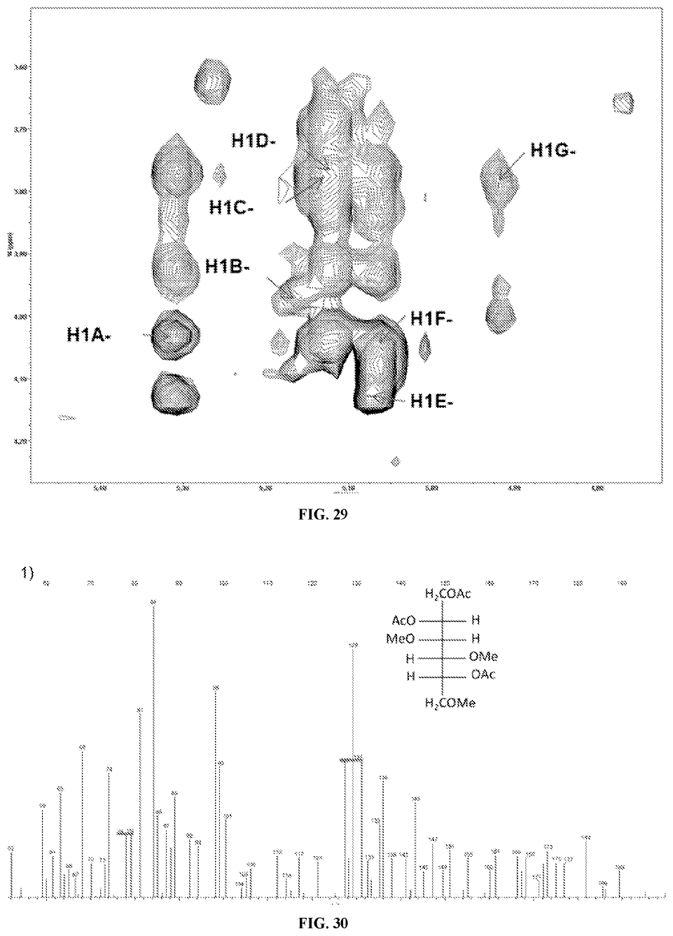

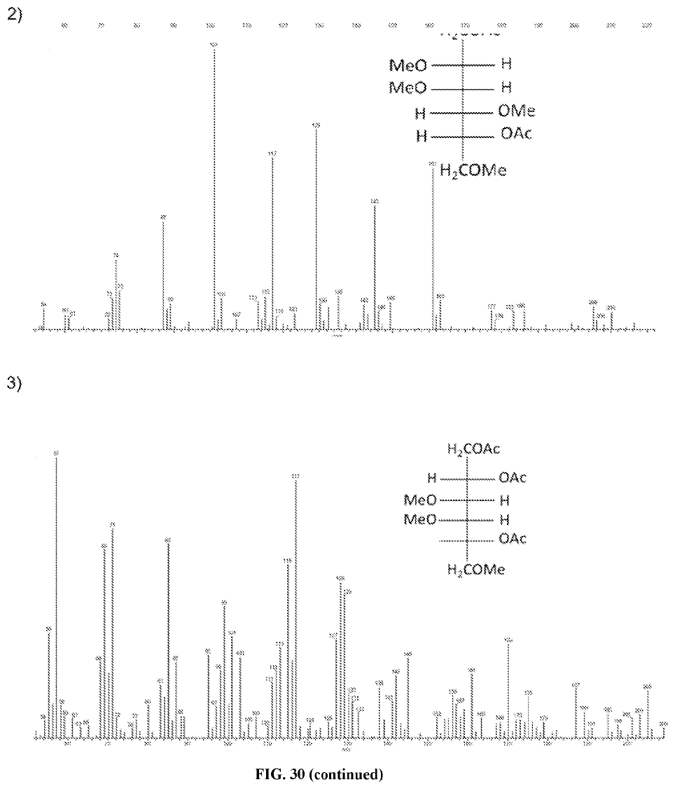

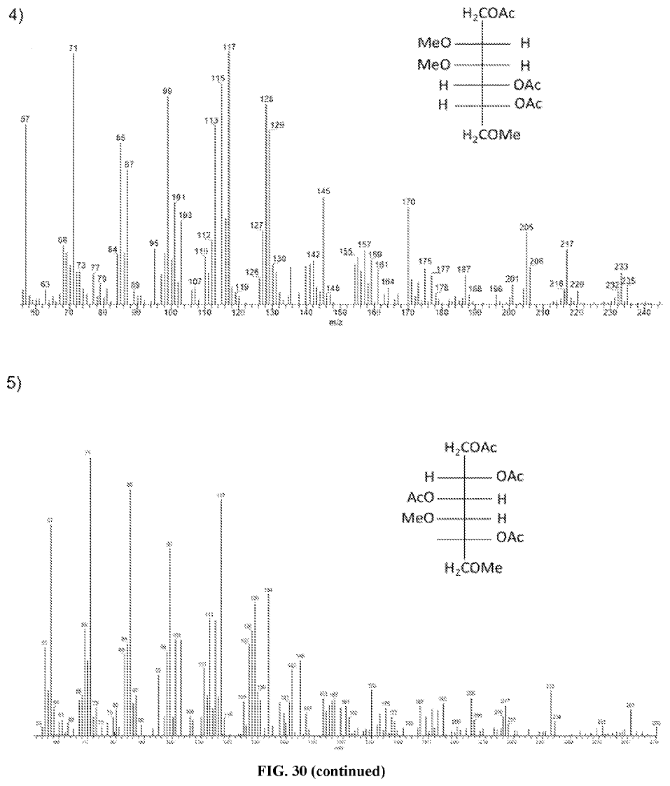

In order to confirm the results obtained by NMR, an analysis by GC-MS of the monosaccharides derived from the purified EPS was performed. The results obtained are presented in FIG. 30 and Table 48.

TABLE 48
GC-MS analysis of monosaccharides derived from purified EPS

| | Methylated and Acetylated | Retention | | |
|---|---|---|---|---|
| | Derivative | time (min) | Type of Union | Mass Fragments (m/Z) |
| 1 | 1,2,5-Tri-O-acetyl-3,4,6-tri-O- | 22.16 | →2)-Glcp-(1→ | 189, 161, 129, 87 |
| | methyl-D-glucose | | | |
| 2 | 1,5-Di-O-acetyl-2,3,4,6-tetra-O- | 22.77 | Manp-(1→ | 205, 161, 145, 129, |
| | methyl-D-manosa | | | 101, 87 |
| 3 | 1,2,5-Tri-O-acetyl-4,3,6-tri-O- | 25.48 | →2)-Galp-(1→ | 189, 161, 145, 129, |
| | methyl-D-galactosa | | | 101 |
| 4 | 1,4,5-Tri-O-acetyl-2,3,6-tri-O- | 25.54 | →4)-Manp-(1→ | 233, 117, 101, 99 |
| | methyl-D-manosa | | | |
| 5 | 1,2,3,5-Tetra-O-acetyl-4,6-di-O- | 25.65 | →2)-3)-Galp-(1→ | 261, 202, 161, 129, |
| | methyl-D-galactose | | | 101, 87 |

The mass spectra of the identified species confirmed the presence of monosaccharides previously assigned by NMR and HPAEC-PAD. Additionally, these results provided information on the connectivity between EPS residues which is consistent with NMR results.

### Conclusions and Model

From the results, the following model of the EPS structure was obtained:

This model satisfies the experimental data of NMR, GC-MS, HPAEC-PAD, and the relationships of the monosaccharide constituents of EPS. Although the data indicated the presence of glucosamine in the polysaccharide, it was not possible to obtain experimental information that demonstrates its connectivity with the rest of the molecule. This is mainly due to its low ratio in EPS (1:16 with respect to Mannose) and its low signal intensity in NMR experiments (e.g., FIG. 23 and FIG. 27).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A composition comprising a microorganism and at least one seed formulation component, wherein the microorganism is selected from Klebsiella sp., Bacillus cereus, Exiguobacterium undeae, or a combination thereof.

2. The composition of claim 1, wherein the seed formulation component is an adjuvant, an additive, or a stabilizer.

3. The composition of claim 1 or claim 2, wherein the seed formulation component is selected from one or more of polyvinylpyrrolidone (PVP), gum Arabic, and Xanthan gum.

4. The composition of any one of claims 1 to 3, further comprising one or more of peptone, tryptone, or meat extract.

5. The composition of any one of claims 1 to 4, wherein the microorganism is present at a concentration of greater than about 1×10⁸ CFU/ml.

6. The composition of any one of claims 1 to 4, wherein the microorganism is present at a concentration of from about 1×10⁴ CFU/ml to about 1×10¹⁰ CFU/ml.

7. The composition of any one of claims 1 to 6, wherein the microorganism is selected from Klebsiella aerogenes, Bacillus cereus, Exiguobacterium undeae, or a combination thereof.

8. The composition of claim 7, wherein the Klebsiella aerogenes is a strain CK1 or a derivative thereof.

9. The composition of claim 8, wherein the strain CK1 has a DSMZ accession number DSM 34332.

10. The composition of any one of claims 7 to 9, wherein the Bacillus cereus is a strain CK2 or a derivative thereof.

11. The composition of claim 10, wherein the strain CK2 has a DSMZ accession number DSM 34322.

12. The composition of any one of claims 7 to 11, wherein the Exiguobacterium undeae, is a strain CK3 or a derivative thereof.

13. The composition of claim 12, wherein the strain CK3 has a DSMZ accession number DSM 34323.

14. The composition of any one of claims 1 to 13, wherein the composition has a shelf life of at least about 6 months.

15. The composition of any one of claims 1 to 14, wherein the composition is a liquid.

16. The composition of any one of claims 1 to 15, wherein the composition confers anti-fungal activity.

17. The composition of claim 16, wherein the anti-fungal activity is against one or more of Macrophomina phaseolina, Fusarium sp., Fusarium tucumaniae, Septoria sp., or Sclerotinia sclerotiorum.

18. The composition of any one of claims 1 to 17, wherein the composition confers plant growth regulatory activity.

19. The composition of any one of claims 1 to 18, wherein the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification.

20. The composition of claim 19, wherein the composition comprises Klebsiella aerogenes and the Klebsiella aerogenes comprises a nitrogen pathway signature gene set forth in Table 25.

21. The composition of any one of claims 1 to 20, wherein the composition comprises Klebsiella aerogenes and the Klebsiella aerogenes comprises a phosphate solubilization signature gene set forth in Table 29.

22. The composition of any one of claims 1 to 21, wherein the composition comprises Klebsiella aerogenes and the Klebsiella aerogenes comprises a plant growth regulatory signature gene set forth in Table 35.

23. The composition of any one of claims 1 to 22, wherein the composition comprises Klebsiella aerogenes and the Klebsiella aerogenes does not produce carbapenemase (KPC), metallo-beta-lactamases (MLB), or oxacillinase (Oxa).

24. The composition of any one of claims 1 to 23, wherein the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification.

25. The composition of claim 24, wherein the Bacillus cereus comprises a nitrogen pathway signature gene set forth in Table 26.

26. The composition of any one of claims 1 to 25, wherein the composition comprises Bacillus cereus and the Bacillus cereus comprises a phosphate solubilization signature gene set forth in Table 29.

27. The composition of any one of claims 1 to 26, wherein the composition comprises Bacillus cereus and the Bacillus cereus comprises a plant growth regulatory signature gene set forth in Table 46.

28. The composition of any one of claims 1 to 27, comprising a combination of Klebsiella aerogenes and Bacillus cereus in a 50:50 ratio (CFU/CFU).

29. The composition of any one of claims 1 to 28, wherein the composition is suitable to be applied to the seeds in combination with a second seed treatment optionally comprising a nutrient or a pesticide.

30. The composition of any one of claims 1 to 29, wherein the composition is suitable to be applied to the seeds with a liquid or solid carrier.

31. The composition of any one of claims 1 to 30, wherein the microorganism is present as a granule, a capsule, a dust, a powder, a slurry, a film, a liquid suspension, or a combination thereof.

32. A composition comprising a microorganism isolated from a plant growing in the high desert and at least one seed formulation component.

33. The composition of claim 32, wherein the microorganism is isolated from a plant growing in Puna de Atacama.

34. The composition of claim 32 or claim 33, wherein the microorganism is isolated from a soil, a sediment, or a rhizosphere of the plant.

35. The composition of any one of claims 32 to 34, wherein the bacterium is of the genus Klebsiella, Bacillus, or Exiguobacterium.

36. The composition of any one of claims 32 to 35, wherein the bacterium is Klebsiella aerogenes, Bacillus cereus, or Exiguobacterium undeae.

37. The composition of claim 36, wherein the Klebsiella aerogenes is a strain CK1 or a derivative thereof.

38. The composition of claim 37, wherein the strain CK1 has a DSMZ accession number DSM 34332.

39. The composition of any one of claims 36 to 38, wherein the Bacillus cereus is a strain CK2 or a derivative thereof.

40. The composition of claim 39, wherein the strain CK2 has a DSMZ accession number DSM 34322.

41. The composition of any one of claims 36 to 40, wherein the Exiguobacterium undeae is a strain CK3 or a derivative thereof.

42. The composition of claim 41, wherein the strain CK3 has a DSMZ accession number DSM 34323.

43. The composition of any one of claims 32 to 40, wherein the seed formulation component is selected from one or more of polyvinylpyrrolidone (PVP), gum Arabic, and Xanthan gum.

44. The composition of any one of claims 32 to 41, further comprising one or more of peptone, tryptone, or meat extract.

45. The composition of any one of claims 32 to 44, wherein the microorganism is present at a concentration of greater than about 1×10⁸ CFU/ml.

46. The composition of any one of claims 32 to 44, wherein the microorganism is present at a concentration of from about 1×10⁴ CFU/ml to about 1×10¹⁰ CFU/ml.

47. The composition of any one of claims 32 to 45, wherein the composition has a shelf life of at least about 6 months.

48. The composition of any one of claims 32 to 46, wherein the composition is a liquid.

49. The composition of any one of claims 32 to 48, wherein the composition confers anti-fungal activity.

50. The composition of claim 49, wherein the anti-fungal activity is against one or more of Macrophomina phaseolina, Fusarium sp., Fusarium tucumaniae, Septoria sp., or Sclerotinia sclerotiorum.

51. The composition of any one of claims 32 to 50, wherein the composition confers plant growth regulatory activity.

52. The composition of any one of claims 32 to 51, wherein the seed treatment comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification.

53. The composition of claim 52, wherein the composition comprises Klebsiella aerogenes and the Klebsiella aerogenes comprises a nitrogen pathway signature gene set forth in Table 25.

54. The composition of any one of claims 32 to 53, wherein the composition comprises Klebsiella aerogenes and the Klebsiella aerogenes comprises a phosphate solubilization signature gene set forth in Table 29.

55. The composition of any one of claims 32 to 54, wherein the composition comprises Klebsiella aerogenes and the Klebsiella aerogenes comprises a plant growth regulatory signature gene set forth in Table 35.

56. The composition of any one of claims 32 to 55, wherein the composition comprises Klebsiella aerogenes and the Klebsiella aerogenes does not produce carbapenemase (KPC), metallo-beta-lactamases (MLB), or oxacillinase (Oxa).

57. The composition of any one of claims 32 to 56, wherein the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification.

58. The composition of claim 57, wherein the Bacillus cereus comprises a nitrogen pathway signature gene set forth in Table 26.

59. The composition of any one of claims 32 to 58, wherein the composition comprises Bacillus cereus and the Bacillus cereus comprises a phosphate solubilization signature gene set forth in Table 29.

60. The composition of any one of claims 32 to 59, wherein the composition comprises Bacillus cereus and the Bacillus cereus comprises a plant growth regulatory signature gene set forth in Table 46.

61. The composition of any one of claims 32 to 60, comprising a combination of Klebsiella aerogenes and Bacillus cereus in a 50:50 ratio (CFU/CFU).

62. The composition of any one of claims 32 to 61, wherein the composition is suitable to be applied to the seeds in combination with a second seed treatment, optionally comprising a nutrient or a pesticide.

63. The composition of any one of claims 32 to 62, wherein the composition is suitable to be applied to the seeds with a liquid or a solid carrier.

64. The composition of any one of claims 32 to 63, wherein the microorganism is present as a granule, a capsule, a dust, a powder, a slurry, a film, a liquid suspension, or a combination thereof.

65. The composition of any one of claims 1 to 64, wherein the composition further comprises water.

66. A treated seed comprising a plant seed and the composition of any one of claims 1 to 65.

67. A plant grown from the treated seed of claim 66.

68. A method of controlling fungal growth, the method comprising contacting a plant seed to the composition of any one of claims 1 to 65; and germinating the plant seed under a condition capable of exposing the plant seed to a fungus, whereby the seed treatment reduces growth of the fungus on or around the plant seed.

69. A method of protecting plant health, the method comprising contacting a plant seed to the composition of any one of claims 1 to 65; whereby germination rate, quality of germinated seed, or a combination thereof is improved as compared to an untreated plant seed.

70. A method of increasing crop yield, the method comprising contacting a set of plant seeds to the composition of any one of claims 1 to 65; planting the set: growing plants from the planted set to harvest; and harvesting the plants or a portion thereof, wherein the crop yield is increased as compared to crop yield from an untreated set of plant seeds.

71. A method of promoting growth of a plant, the method comprising contacting seed of the plant to the composition of any one of claims 1 to 65; germinating the seed of the plant; and growing the resulting plant for a time period sufficient to develop leaves and roots, whereby biomass of the plant, root development of the plant, or a combination thereof is improved compared to a plant grown from an untreated seed.

72. The method of claim 71, wherein root development comprises length of the roots, number of lateral roots, or a combination thereof.

73. A method of increasing fertility of a soil, the method comprising contacting a plurality of seeds of a plant to the composition of any one of claims 1 to 65; planting the plurality of seeds in the soil; and growing a plurality of plants grown from the plurality of seeds in the soil, thereby increasing the fertility of the soil.

74. The method of any one of claims 68 to 73, wherein the composition is contacted to the seeds in a liquid or solid carrier.

75. The method of any one of claims 68 to 74, wherein the composition is contacted to the seeds in combination with a second seed treatment composition.

76. The method of claim 75, wherein the second seed treatment composition comprises a nutrient or a pesticide.

77. The method of any one of claims 68 to 76, wherein the seeds are monocotyledons.

78. The method of any one of claims 68 to 76, wherein the seeds are dicotyledons.

79. The method of any one of claims 68 to 78, wherein the seeds are soybean seeds, corn seeds, wheat seeds, or a combination thereof.

80. The method of any one of claims 68 to 79, wherein the composition is contacted to the seeds by a means selected from aerosol application, spray-dried application, liquid application, powder application, mist application, atomized application, semi-solid application, gel application, coating application, lotion application, linked or linker material application, material application, in-furrow application, spray application, irrigation, injection, dusting, pelleting, coating of the plant, coating of the plant seed, or coating of the planting medium.

81. A method of preparing a seed treatment, the method comprising growing a microorganism selected from a genus of Klebsiella, Bacillus, Exiguobacterium, or a combination thereof to from about 1×10⁴ CFU to about 1×10¹⁰ CFU/g in a liquid media; and preparing a composition comprising a liquid media, the microorganism, at least one formulation component selected from polyvinylpyrrolidone (PVP), gum Arabic, and Xanthan gum.

82. The method of claim 81, wherein the microorganism is Klebsiella aerogenes, Bacillus cereus, Exiguobacterium undeae, or a combination thereof.

83. The method of claim 82, wherein the Klebsiella aerogenes is a strain CK1 or a derivative thereof.

84. The method of claim 83, wherein the strain CK1 has a DSMZ accession number DSM 34332.

85. The method of any one of claims 82 to 84, wherein the Bacillus cereus is a strain CK2 or a derivative thereof.

86. The method of claim 85, wherein the strain CK2 has a DSMZ accession number DSM 34322.

87. The method of any one of claims 82 to 86, wherein the Exiguobacterium undeae is a strain CK3 or a derivative thereof.

88. The method of claim 87, wherein the strain CK3 has a DSMZ accession number DSM 34323.

89. The method of any one of claims 81 to 88, further comprising applying the seed treatment to a plant seed.

90. The method of any one of claims 81 to 89, wherein the liquid media comprises one or more of peptone, tryptone, or meat extract.

91. The method of any one of claims 81 to 90, wherein the microorganism is present at a concentration of greater than 1×10⁸ CFU/ml.

92. The method of any one of claims 81 to 90, wherein the microorganism is present at a concentration of from about 1×10⁴ CFU/ml to about 1×10¹⁰ CFU/ml.

93. The method of any one of claims 81 to 92, wherein the microorganism is present at a concentration of greater than 1×10⁸ CFU/ml for at least 6 months at 25° C.

94. The method of any one of claims 81 to 92, wherein the microorganism is present at a concentration of greater than 1×10⁸ CFU/ml for at least 6 months at a temperature from about 20° C. to about 35° C.

95. The method of any one of claims 81 to 92, wherein the microorganism is present at a concentration of from about 1×10⁴ CFU/ml to about 1×10¹⁰ CFU/ml for at least 6 months at 25° C.

96. The method of any one of claims 81 to 92, wherein the microorganism is present at a concentration of from about 1×10⁴ CFU/ml to about 1×10¹⁰ CFU/ml for at least 6 months at a temperature from about 20° C. to about 35° C.

97. A composition comprising a microorganism and at least one soil or plant amendment component, wherein the microorganism is selected from Klebsiella, Bacillus cereus, Exiguobacterium undeae, or a combination thereof.

98. The composition of claim 97, wherein the soil or plant amendment comprises polyvinylpyrrolidone (PVP), gum Arabic, or Xanthan gum.

99. The composition of claim 97 or claim 98, further comprising one or more of peptone, tryptone, or meat extract.

100. The composition of any one of claims 97 to 99, wherein the microorganism is present at a concentration of greater than about 1×10⁸ CFU/ml.

101. The composition of any one of claims 97 to 99, wherein the microorganism is present at a concentration of from about 1×10⁴ CFU/ml to about 1×10¹⁰ CFU/ml.

102. The composition of any one of claims 96 to 101, wherein the microorganism is selected from Klebsiella aerogenes, Bacillus cereus, Exiguobacterium undeae, or a combination thereof.

103. The composition of claim 102, wherein the Klebsiella aerogenes is a strain CK1 or a derivative thereof.

104. The composition of claim 103, wherein the strain CK1 has a DSMZ accession number DSM 34332.

105. The composition of any one of claims 102 to 104, wherein the Bacillus cereus is a strain CK2 or a derivative thereof.

106. The composition of claim 105, wherein the strain CK2 has a DSMZ accession number DSM 34322.

107. The composition of any one of claims 102 to 106, wherein the Exiguobacterium undeae, is a strain CK3 or a derivative thereof.

108. The composition of claim 107, wherein the strain CK3 has a DSMZ accession number DSM 34323.

109. The composition of any one of claims 97 to 10⁸, wherein the composition has a shelf life of at least about 6 months.

110. The composition of any one of claims 97 to 109, wherein the composition is a liquid.

111. The composition of any one of claims 97 to 110, wherein the composition confers anti-fungal activity.

112. The composition of claim 111, wherein the anti-fungal activity is against one or more of Macrophomina phaseolina, Fusarium sp., Fusarium tucumaniae, Septoria sp., or Sclerotinia sclerotiorum.

113. The composition of any one of claims 97 to 112, wherein the composition confers plant growth regulatory activity.

114. The composition of any one of claims 97 to 113, wherein the composition comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification.

115. The composition of claim 114, wherein the composition comprises Klebsiella aerogenes and the Klebsiella aerogenes comprises a nitrogen pathway signature gene set forth in Table 25.

116. The composition of any one of claims 97 to 115, wherein the composition comprises Klebsiella aerogenes and the Klebsiella aerogenes comprises a phosphate solubilization signature gene set forth in Table 29.

117. The composition of any one of claims 97 to 116, wherein the composition comprises Klebsiella aerogenes and the Klebsiella aerogenes comprises a plant growth regulatory signature gene set forth in Table 35.

118. The composition of any one of claims 97 to 117, wherein the composition comprises Klebsiella aerogenes and the Klebsiella aerogenes does not produce carbapenemase (KPC), metallo-beta-lactamases (MLB), or oxacillinase (Oxa).

119. The composition of any one of claims 97 to 118, wherein the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification.

120. The composition of claim 119, wherein the Bacillus cereus comprises a nitrogen pathway signature gene set forth in Table 26.

121. The composition of any one of claims 97 to 120, wherein the composition comprises Bacillus cereus and the Bacillus cereus comprises a phosphate solubilization signature gene set forth in Table 29.

122. The composition of any one of claims 97 to 121, wherein the composition comprises Bacillus cereus and the Bacillus cereus comprises a plant growth regulatory signature gene set forth in Table 46.

123. The composition of any one of claims 97 to 122, comprising a combination of Klebsiella aerogenes and Bacillus cereus in a 50:50 ratio (CFU/CFU).

124. The composition of any one of claims 97 to 123, wherein the composition is suitable for application with a second treatment, optionally comprising a nutrient, a pesticide, or another seed treatment.

125. The composition of any one of claims 97 to 124, wherein the composition further comprises a liquid or a solid carrier.

126. The composition of any one of claims 97 to 125, wherein the microorganism is present as a granule, a capsule, a dust, a powder, a slurry, a film, a liquid suspension, or a combination thereof.

127. A composition comprising a microorganism isolated from a plant growing in the high desert and at least one soil or plant amendment component.

128. The composition of claim 127, wherein the microorganism is isolated from a plant growing in Puna de Atacama.

129. The composition of claim 127 or claim 128, wherein the microorganism is isolated from a soil, a sediment, or a rhizosphere of the plant.

130. The composition of any one of claims 127 to 129, wherein the bacterium is of the genus Klebsiella, Bacillus, or Exiguobacterium.

131. The composition of any one of claims 127 to 130, wherein the bacterium is Klebsiella aerogenes, Bacillus cereus, or Exiguobacterium undeae.

132. The composition of any one of claims 127 to 131, wherein the Klebsiella aerogenes is a strain CK1 or a derivative thereof.

133. The composition of claim 132, wherein the strain CK1 has a DSMZ accession number DSM 34332.

134. The composition of any one of claims 127 to 133, wherein the Bacillus cereus is a strain CK2 or a derivative thereof.

135. The composition of claim 134, wherein the strain CK2 has a DSMZ accession number DSM 34322.

136. The composition of any one of claims 127 to 135, wherein the Exiguobacterium undeae is a strain CK3 or a derivative thereof.

137. The composition of claim 136, wherein the strain CK3 has a DSMZ accession number DSM 34323.

138. The composition of any one of claims 127 to 137, wherein the soil or plant amendment component comprises polyvinylpyrrolidone (PVP), gum Arabic, or Xanthan gum.

139. The composition of any one of claims 127 to 138, further comprising one or more of peptone, tryptone, or meat extract.

140. The composition of any one of claims 127 to 139, wherein the microorganism is present at a concentration of greater than about 1×10⁸ CFU/ml.

141. The composition of any one of claims 127 to 139, wherein the microorganism is present at a concentration of from about 1×10⁴ CFU/ml to about 1×10¹⁰ CFU/ml.

142. The composition of any one of claims 127 to 141, wherein the composition has a shelf life of at least about 6 months.

143. The composition of any one of claims 127 to 142, wherein the composition is a liquid.

144. The composition of any one of claims 127 to 143, wherein the composition confers anti-fungal activity.

145. The composition of claim 144, wherein the anti-fungal activity is against one or more of Macrophomina phaseolina, Fusarium sp., Fusarium tucumaniae Septoria sp., or Sclerotinia sclerotiorum.

146. The composition of any one of claims 127 to 145, wherein the composition confers plant growth regulatory activity.

147. The composition of any one of claims 127 to 146, wherein the composition comprises Klebsiella aerogenes and wherein the Klebsiella aerogenes comprises a nitrogen pathway signature comprises at least one of assimilatory nitrogen reduction: dissimilatory nitrate reduction; and the absence of all or a portion of nitrogen fixation, nitrification, and denitrification.

148. The composition of claim 147, wherein the composition comprises Klebsiella aerogenes and the Klebsiella aerogenes comprises a nitrogen pathway signature gene set forth in Table 25.

149. The composition of any one of claims 127 to 148, wherein the composition comprises Klebsiella aerogenes and the Klebsiella aerogenes comprises a phosphate solubilization signature gene set forth in Table 29.

150. The composition of any one of claims 127 to 149, wherein the composition comprises Klebsiella aerogenes and the Klebsiella aerogenes comprises a plant growth regulatory signature gene set forth in Table 35.

151. The composition of any one of claims 127 to 150, wherein the composition comprises Klebsiella aerogenes and the Klebsiella aerogenes does not produce carbapenemase (KPC), metallo-beta-lactamases (MLB), or oxacillinase (Oxa).

152. The composition of any one of claims 127 to 151, wherein the composition comprises Bacillus cereus and the Bacillus cereus comprises a nitrogen pathway signature comprises at least one of assimilatory nitrogen reduction: dissimilatory nitrate reduction; and the absence of all or a portion of nitrogen fixation, nitrification, and denitrification.

153. The composition of claim 152, wherein the Bacillus cereus comprises a nitrogen pathway signature gene set forth in Table 26.

154. The composition of any one of claims 127 to 153, wherein the composition comprises Bacillus cereus and the Bacillus cereus comprises a phosphate solubilization signature gene set forth in Table 29.

155. The composition of any one of claims 127 to 154, wherein the composition comprises Bacillus cereus and the Bacillus cereus comprises a plant growth regulatory signature gene set forth in Table 46.

156. The composition of any one of claims 127 to 155, comprising a combination of Klebsiella aerogenes and Bacillus cereus in a 50:50 ratio (CFU/CFU).

157. The composition of any one of claims 127 to 156, wherein the composition is suitable for application with a second treatment, optionally comprising a nutrient, a pesticide, or another seed treatment.

158. The composition of any one of claims 127 to 157, wherein the composition further comprises a liquid or a solid carrier.

159. The composition of any one of claims 127 to 158, wherein the microorganism is present as a granule, a capsule, a dust, a powder, a slurry, a film, a liquid suspension, or a combination thereof.

160. The composition of any one of claims 97 to 159, wherein the composition further comprises water.

161. A plant grown with the composition of any one of claims 97 to 160.

162. A method of controlling fungal growth, the method comprising contacting a plant to the composition of any one of claims 96 to 160; and growing the plant under a condition capable of exposing the plant to a fungus, whereby the composition reduces growth of the fungus on or around the plant.

163. A method of protecting plant health, the method comprising contacting a plant to the composition of any one of claims 96 to 160; whereby plant growth is improved as compared to an untreated plant.

164. A method of increasing crop yield, the method comprising contacting a set of plants to the composition of any one of claims 96 to 160; growing plants from the set of plants to harvest; and harvesting the plants or a portion thereof, wherein the crop yield is increased as compared to crop yield from an untreated set of plants.

165. A method of promoting growth of a plant, the method comprising contacting the plant to the composition of any one of claims 96 to 160: growing the plant for a time period sufficient to develop leaves and roots, whereby biomass of the plant, root development of the plant, or a combination thereof is improved compared to an untreated plant.

166. The method of claim 165, wherein root development comprises length of the roots, number of lateral roots, or a combination thereof.

167. A method of increasing fertility of a soil, the method comprising contacting a plurality of plants to the composition of any one of claims 96 to 160; and growing a plurality of plants in the soil, thereby increasing the fertility of the soil.

168. The method of any one of claims 162 to 167, wherein the composition is contacted to the plants in a liquid or solid carrier.

169. The method of any one of claims 162 to 168, wherein the composition is contacted to the plants in combination with a second soil or plant amendment composition.

170. The method of claim 169, wherein the second soil or plant amendment composition comprises a nutrient or a pesticide.

171. The method of any one of claims 162 to 170, wherein the plants are monocotyledons.

172. The method of any one of claims 162 to 170, wherein the plants are dicotyledons.

173. The method of any one of claims 162 to 172, wherein the plants are soybean, corn, wheat, or a combination thereof.

174. The method of any one of claims 68 to 173, wherein the composition is contacted to the plants by a means selected from aerosol application, spray-dried application, liquid application, powder application, mist application, atomized application, semi-solid application, gel application, coating application, lotion application, linked or linker material application, material application, in-furrow application, spray application, irrigation, injection, dusting, pelleting, coating of the plant, coating of the plant seed, or coating of the planting medium.

175. A method of preparing a soil or plant amendment, the method comprising growing a microorganism selected from a genus of Klebsiella, Bacillus, Exiguobacterium, or a combination thereof to at least 1×10⁸ CFU/g in a liquid media; and preparing a composition comprising a liquid media, the microorganism, at least one formulation component selected from polyvinylpyrrolidone (PVP), gum Arabic, and Xanthan gum.

176. The method of claim 175, wherein the microorganism is Klebsiella aerogenes, Bacillus cereus, Exiguobacterium undeae, or a combination thereof.

177. The method of claim 176, wherein the Klebsiella aerogenes is a strain CK1 or a derivative thereof.

178. The method of claim 177, wherein the strain CK1 has a DSMZ accession number DSM 34332.

179. The method of any one of claims 175 to 178, wherein the Bacillus cereus is a strain CK2 or a derivative thereof.

180. The method of claim 179, wherein the strain CK2 has a DSMZ accession number DSM 34322.

181. The method of any one of claims 175 to 180, wherein the Exiguobacterium undeae is a strain CK3 or a derivative thereof.

182. The method of claim 181, wherein the strain CK3 has a DSMZ accession number DSM 34323.

183. The method of any one of claims 175 to 182, further comprising applying the seed treatment to a plant seed.

184. The method of any one of claims 175 to 183, wherein the liquid media comprises one or more of peptone, tryptone, or meat extract.

185. The method of any one of claims 175 to 184, wherein the microorganism is present at a concentration of greater than 1×10⁸ CFU/ml.

186. The method of any one of claims 175 to 184, wherein the microorganism is present at a concentration of from about 1×10⁴ CFU/ml to about 1×10¹⁰ CFU/ml.

187. The method of any one of claims 175 to 186, wherein the microorganism is present at a concentration of greater than 1×10⁸ CFU/ml for at least 6 months at 25° C.

188. The method of any one of claims 175 to 186, wherein the microorganism is present at a concentration of greater than 1×10⁸ CFU/ml for at least 6 months at a temperature from about 20° C. to about 35° C.

189. The method of any one of claims 175 to 186, wherein the microorganism is present at a concentration of from about 1×10⁴ CFU/ml to about 1×10¹⁰ CFU/ml for at least 6 months at 25° C.

190. The method of any one of claims 175 to 186, wherein the microorganism is present at a concentration of from about 1×10⁴ CFU/ml to about 1×10¹⁰ CFU/ml for at least 6 months at a temperature from about 20° C. to about 35° C.
